(19) United States
(12) Patent Application Publication
(10) Pub. No.: US 2026/0271617 A1
(43) Pub. Date: Sep. 10, 2026

(54) PLURALITY OF HOST MATERIALS AND ORGANIC ELECTROLUMINESCENT DEVICE COMPRISING THE SAME

(71) Applicant: DuPont Specialty Materials Korea Ltd., Chungcheongnam-do (KR)

(72) Inventors: Sang-Hee CHO, Gyeonggi-do (KR); Young-Jae KIM, Gyeonggi-do (KR); HaeYeon KIM, Gyeonggi-do (KR); Young-Jun CHO, Gyeonggi-do (KR); Kyung-Hoon CHOI, Gyeonggi-do (KR); So-Young JUNG, Gyeonggi-do (KR); Su-Hyun LEE, Gyeonggi-do (KR); Mi-Ja LEE, Gyeonggi-do (KR)

(21) Appl. No.: 19/659,800

(22) Filed: Apr. 27, 2026

Related U.S. Application Data
(63) Continuation of application No. 17/890,926, filed on Aug. 18, 2022, now Pat. No. 12,666,865.

(30) Foreign Application Priority Data
Aug. 27, 2021 (KR) 10-2021-0113823
Jan. 5, 2022 (KR) 10-2022-0001374
Jun. 24, 2022 (KR) 10-2022-0077232

(51) Int. Cl. H10K 85/60; C07D 209/56; C07D 263/52; C07D 307/91; C07D 405/10; C07D 413/12; C07D 413/14; C07D 487/16; C09K 11/06; H10K 50/11; H10K 101/00; H10K 101/10; H10K 101/30

(52) U.S. Cl. CPC H10K 85/636; C07D 209/56; C07D 263/52; C07D 307/91; C07D 405/10; C07D 413/12; C07D 413/14; C07D 487/16; C09K 11/06; H10K 85/615; H10K 85/633; H10K 85/654; H10K 85/6572; H10K 85/6574; C09K 2211/1018; H10K 50/11; H10K 85/622; H10K 85/657; H10K 85/6576; H10K 2101/10; H10K 2101/30; H10K 2101/90

(57) ABSTRACT

The present disclosure relates to three different types of host materials, and an organic electroluminescent device comprising the same, and it is possible to provide an organic electroluminescent device having higher luminous efficiency and/or improved lifetime properties compared to a conventional organic electroluminescent device.

### CLAIM OF BENEFIT OF PRIOR APPLICATION

This application is a continuation of U.S. patent application Ser. No. 17/890,926, filed Aug. 18, 2022, which is incorporated by reference herein in its entirety.

### TECHNICAL FIELD

The present disclosure relates to a plurality of host materials and an organic electroluminescent device comprising the same.

### BACKGROUND ART

In 1987, Tang et al. of Eastman Kodak first developed a small molecule green organic electroluminescent device (OLED) of TPD/Alq3 bilayer consisting of a light-emitting layer and a charge transport layer. Since then, the research on an OLED has been rapidly carried out, and it has been commercialized. At present, phosphorescent materials, which provide excellent luminous efficiency in realizing panels, are mainly used in OLEDs. In many applications such as TVs and lightings, the lifetime of OLEDs is insufficient and higher efficiency of OLEDs is still required. Typically, the higher the luminance of an OLED, the shorter the lifetime that the OLED has. Thus, an OLED which has high luminous efficiency and/or long lifetime is required for long time uses and high resolution of displays.

In order to enhance luminous efficiency, driving voltage and/or lifetime, various materials or concepts for an organic layer of an OLED have been proposed. However, they were not satisfactory in practical use.

U.S. Pat. No. 9,705,099 discloses an OLED using a carbazolyl-containing compound as three types of host materials. However, the aforementioned reference does not specifically disclose the specific combination of host materials claimed in the present disclosure. In addition, there is still a need to develop host materials for improving OLED performance.

### DISCLOSURE OF INVENTION

### Technical Problem

The objective of the present disclosure is firstly, to provide a plurality of host materials capable of producing an organic electroluminescent device having low driving voltage, high luminous efficiency, and/or long lifetime properties, and secondly, to provide an organic electroluminescent device comprising the host materials.

The objective of the present disclosure is to provide an organic electroluminescent device having low driving voltage, high luminous efficiency and/or improved lifetime properties while improving the charge balance in a light-emitting layer by facilitating control of the energy level and mobility of a phosphorescent host material, by comprising at least three different types of host materials including a specific combination of compounds.

### Solution to Problem

As a result of intensive studies to solve the technical problems, the present inventors have found that the above objective can be achieved by a plurality of host materials comprising a first host material, a second host material, and a third host material, wherein each of the first host material, the second host material and the third host material does not comprise a carbazole or fused carbazole structure, and the first host material, the second host material and the third host material are different from each other.

In addition, the present inventors have discovered that when the combination of a compound represented by formula 1 of the present disclosure, a compound represented by any one of formulas 2 to 4 of the present disclosure, and a third compound different from the compounds and represented by any one of formulas of 1 to 4 of the present disclosure is used in a light-emitting layer, hole and electronic properties are better balanced by appropriate HOMO (highest occupied molecular orbital) and LUMO (lowest unoccupied molecular orbital) energy levels, thereby providing an OLED having lower driving voltage, higher luminous efficiency and/or longer lifetime properties compared to a conventional OLED.

### Advantageous Effects of Invention

An organic electroluminescent device having lower driving voltage, higher luminous efficiency, and/or improved lifespan properties compared to a conventional organic electroluminescent device is provided by comprising three different types of host materials according to the present disclosure, and it is possible to produce a display system or lighting system using the same.

### BRIEF DESCRIPTION OF THE FIGURES

FIG. 1 illustrates a graph showing the PL (photoluminescence) results of thin film of an organic electroluminescent material according to an embodiment of the present disclosure.

FIG. 2 illustrates a diagram schematically showing a HOMO energy diagram of a light-emitting layer of an organic electroluminescent device according to an embodiment of the present disclosure.

FIG. 3 illustrates a diagram schematically showing a LUMO energy diagram of a light-emitting layer of an organic electroluminescent device according to an embodiment of the present disclosure.

### MODE FOR THE INVENTION

Hereinafter, the present disclosure will be described in detail. However, the following description is intended to explain the present disclosure and is not meant in any way to restrict the scope of the present disclosure.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of organic electroluminescent materials” in the present disclosure means organic electroluminescent materials comprising a combination of at least three types of compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials is a combination of at least three types of materials, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. These at least three types of compounds may be included in the same layer or different layers through common methods used in the art. For example, the at least three materials may be mixture-evaporated or co-evaporated, or may be individually evaporated.

All three types of host materials used herein may be a material having strong hole transport properties or a material having strong electron transport properties. It is preferable to use a material having strong hole transport properties for the first host material, a material having strong electron transport properties for the second host material, and a material having both strong hole transport property and strong electron transport property or only one of the two properties for the third host material.

Herein, the term “fused carbazole structure” refers to a structure in which one or more rings are fused to a carbazole structure, but the structure including a ring formed by fusion to both the two benzene rings and the nitrogen-containing 5-membered ring of carbazole is excluded.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as a heteroaryl substituent, or as substituents in which two heteroaryl substituents are linked. Herein, the substituent(s) of the substituted alkyl, the substituted cycloalkyl, the substituted heterocycloalkyl, the substituted fused ring group of a aliphatic ring(s) and a aromatic ring(s), the substituted aryl, the substituted arylene, the substituted heteroaryl, the substituted heteroarylene, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-alkenylamino, the substituted alkylalkenylamino, the substituted mono- or di-arylamino, the substituted alkylarylamino, the substituted mono- or di-heteroarylamino, the substituted alkylheteroarylamino, the substituted alkenylarylamino, the substituted alkenylheteroarylamino, and the substituted arylheteroarylamino, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl unsubstituted or substituted with a (C6-C30)aryl(s); a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium; a halogen; a cyano; a (C1-C20)alkyl; a (C6-C25)aryl unsubstituted or substituted with a (C1-C20)alkyl(s); a (5- to 25-membered)heteroaryl unsubstituted or substituted with a (C6-C25)aryl(s); and a tri(C6-C25)arylsilyl. According to another embodiment of the present disclosure, the substituent(s), each independently, are at least one selected from the group consisting of deuterium; a halogen; a cyano; a (C1-C10)alkyl; a (C6-C18)aryl unsubstituted or substituted with a (C1-C10)alkyl(s); an unsubstituted (5- to 20-membered)heteroaryl; and a tri(C6-C18)arylsilyl. For example, the substituent(s), each independently, may be one selected from the group consisting of deuterium, a halogen, a cyano, a methyl, a tert-butyl, a phenyl, a naphthyl, a biphenyl, a phenanthrenyl, a dimethylfluorenyl, a pyridyl, a dibenzofuranyl, a dibenzothiophenyl, a benzonaphthofuranyl, a benzonaphthothiophenyl, and a triphenylsilyl, or a combination thereof.

Herein, a ring formed by a linkage of adjacent substituents means that at least two adjacent substituents are linked to or fused with each other to form a substituted or unsubstituted, mono- or polycyclic, (3- to 30-membered) alicyclic or aromatic ring, or the combination thereof. Preferably, the ring may be a substituted or unsubstituted, mono- or polycyclic, (3- to 26-membered) alicyclic or aromatic ring, or the combination thereof. More preferably, the ring may be a mono- or polycyclic, (5- to 25-membered) aromatic ring unsubstituted or substituted with at least one of (C1-C6)alkyl(s), a (C6-C18)aryl(s), and a (3- to 20-membered)heteroaryl(s). In addition, the formed ring may contain at least one heteroatom selected from the group consisting of B, N, O, S, Si, and P, preferably at least one heteroatom selected from the group consisting of N, O, and S. For example, the ring may be a benzene ring unsubstituted or substituted with a phenyl(s), a benzonaphthofuran ring, an acenaphthylene ring, a dihydrodimethylanthracene ring, a cyclopentane ring, an indene ring, an indane ring, a fluorene ring, a phenanthrene ring, an indole ring, a benzofuran ring, a xanthene ring, etc., and the ring may also form a spiro ring.

In the present disclosure, heteroaryl, heteroarylene, and heterocycloalkyl may, each independently, contain at least one heteroatom selected from the group consisting of B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

For example, when using a host having a lower HOMO energy level than a conventional hole type host among the compounds having the core of formula 1, even if there is an advantage in efficiency increase, excess carriers in one direction will occur, which may have an effect such as exciton quenching, resulting in problems of reduced efficiency and lifetime. The present inventors have found that the combination of a hole-type host capable of forming an appropriate HOMO energy level in the core of formula 1 and materials of formulas 2 to 4 having fast electron transport properties results in the hole and electron properties being more balanced by appropriate HOMO and LUMO energy levels, which can provide an OLED having higher luminous efficiency and/or longer lifetime properties compared to a conventional OLED.

To be specific, the present disclosure provides at least three different types of host materials comprising a first host material comprising a compound represented by the following formula 1; a second host material comprising a compound represented by any one of the following formulas 2 to 4; and a third host material belonging to the first or second host material group while having a structure different from those of the first and second host materials.

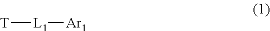

In formula 1,
T represents any one selected from the group consisting of the following formulas 1-1 to 1-5:

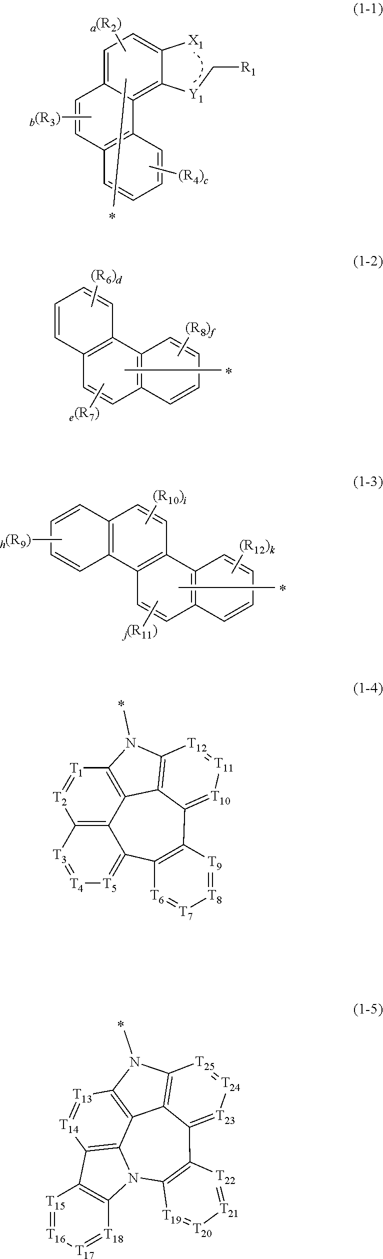

wherein,
X₁ and Y₁, each independently, represent —N═, —NR₅—, —O—, or —S—, with the proviso that any one of X₁ and Y₁ represents —N═, and the other of X₁ and Y₁ represents —NR₅—, —O—, or —S—;
R₁ represents a substituted or unsubstituted (C6-C30)aryl, or a substituted or unsubstituted (3- to 30-membered)heteroaryl;
R₂ to R₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C6-C30)aryl(3- to 30-membered)heteroarylamino, or a substituted or unsubstituted (C1-C30)alkyl(C6-C30)arylamino; or may be linked to an adjacent substituent(s) to form a ring(s);
T₁ to T₂₅, each independently, represent N or CV₁;
V₁, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent(s) to form a ring(s);
a and b, each independently, represent 1 or 2; c represents an integer of 1 to 4; where if each of a to c is an integer of 2 or more, each of R₂ to each of R₄ may be the same or different;
R₆ to R₁₂, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
d, f, h, and k, each independently, represent an integer of 1 to 4; e, i, and j, each independently, represent an integer of 1 or 2; where if each of d to f and h to k is an integer of 2 or more, each of R₆ to each of R₁₁ may be the same or different;
Ar₁ represents a substituted or unsubstituted (C6-C30)aryl, a substituted or unsubstituted (3- to 30-membered)heteroaryl, or

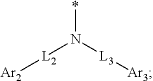

Ar₂ and Ar₃, each independently, represent a substituted or unsubstituted (C6-C30)aryl, a substituted or unsubstituted (3- to 30-membered)heteroaryl, or a substituted or unsubstituted (C3-C30)cycloalkyl; and
L₁ to L₃, each independently, represent a single bond, a substituted or unsubstituted (C6-C30)arylene, or a substituted or unsubstituted (3- to 30-membered)heteroarylene.

According to one embodiment of the present disclosure, in formula 1, X₁ and Y₁, each independently, represent —N═, —O—, or —S—, with the proviso that any one of X₁ and Y₁ represents —N═, the other of X₁ and Y₁ represents —O— or —S—.

According to one embodiment of the present disclosure, R₁ represents a substituted or unsubstituted (C6-C25)aryl, or a substituted or unsubstituted (5- to 25-membered)heteroaryl. According to another embodiment of the present disclosure, R₁ represents an unsubstituted (C6-C18)aryl, or an unsubstituted (5- to 20-membered)heteroaryl. For example, R₁ may be a phenyl, a biphenyl, a quinolyl, an isoquinolyl, etc.

According to one embodiment of the present disclosure, R₂ to R₅, each independently, represent hydrogen.

According to one embodiment of the present disclosure, R₆ to R₁₂, each independently, represent hydrogen, deuterium, a substituted or unsubstituted (C6-C25)aryl, a substituted or unsubstituted (5- to 25-membered)heteroaryl, or a substituted or unsubstituted mono- or di-(C6-C25)arylamino. According to another embodiment of the present disclosure, R₆ to R₁₂, each independently, represent hydrogen, deuterium, a (C6-C28)aryl unsubstituted or substituted with a (C6-C18)aryl(s), an unsubstituted (5- to 20-membered)heteroaryl, or an unsubstituted di(C6-C18)arylamino. For example, R₆ to R₁₂, each independently, may be hydrogen, deuterium, a phenyl, a naphthyl, a phenylnaphthyl, a naphthylphenyl, a phenanthrenyl, a terphenyl, a triphenylenyl, a pyridyl, a quinolyl, a dibenzofuranyl, a dibenzothiophenyl, a diphenylamino, etc.

According to one embodiment of the present disclosure, T₁ to T₂₅, each independently, represent CV₁.

According to one embodiment of the present disclosure, V₁, each independently, represents hydrogen, a substituted or unsubstituted (C6-C25)aryl, or a substituted or unsubstituted (5- to 20-membered)heteroaryl; or adjacent V₁'s may be linked to each other to form a substituted or unsubstituted, mono- or polycyclic, (3- to 30-membered) alicyclic or aromatic ring, or the combination thereof. According to another embodiment of the present disclosure, V₁, each independently, represents hydrogen, an unsubstituted (C6-C18)aryl, or an unsubstituted (5- to 20-membered)heteroaryl; or adjacent V₁'s may be linked to each other to form an unsubstituted (3- to 10-membered) aromatic ring. For example, V₁, each independently, represents hydrogen or phenyl; or adjacent V₁'s may be linked to each other to form an unsubstituted benzene ring.

According to one embodiment of the present disclosure, Ar₁ represents a substituted or unsubstituted (C6-C30)aryl, a substituted or unsubstituted (5- to 20-membered)heteroaryl, or

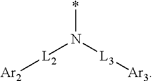

According to another embodiment of the present disclosure, Ar₁ represents a (C6-C30)aryl unsubstituted or substituted with at least one of a (C1-C10)alkyl(s) and a (C6-C18)aryl(s); an unsubstituted (5- to 20-membered)heteroaryl; or

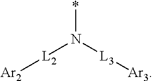

For example, Ar₁ may be a phenyl, a naphthyl, a biphenyl, a phenanthrenyl, a triphenylenyl, a fluoranthenyl, a terphenyl, a dimethylfluorenyl, a diphenylfluorenyl, a dimethylbenzofluorenyl, a diphenylbenzofluorenyl, a spirobifluorenyl, a spiro[cyclopentane-fluoren]yl, a spiro[dihydroindene-fluoren]yl, a spiro[fluorene-benzofluoren]yl, a dibenzofuranyl, a dibenzothiophenyl, a benzonaphthofuranyl, a benzonaphthothiophenyl, etc.

According to one embodiment of the present disclosure, Ar₂ and Ar₃, each independently, represent a substituted or unsubstituted (C6-C25)aryl, or a substituted or unsubstituted (5- to 25-membered)heteroaryl. According to another embodiment of the present disclosure, Ar₂ and Ar₃, each independently, represent a (C6-C25)aryl unsubstituted or substituted with at least one of deuterium, a (C1-C10)alkyl(s), a (5- to 20-membered)heteroaryl(s), and a tri(C6-C18)arylsilyl(s); or a (5- to 20-membered)heteroaryl unsubstituted or substituted with a (C6-C18)aryl(s). For example, Ar₂ and Ar₃, each independently, may be a phenyl unsubstituted or substituted with a tert-butyl(s), a pyridyl(s) or a triphenylsilyl(s); a naphthyl; a naphthylphenyl; a phenylnaphthyl; a biphenyl unsubstituted or substituted with deuterium or a tert-butyl(s); a phenanthrenyl; a terphenyl; a dimethylfluorenyl unsubstituted or substituted with a phenyl(s); a diphenylfluorenyl; a spirobifluorenyl; a pyridyl unsubstituted or substituted with a phenyl(s); a benzofuranyl substituted with a phenyl(s); a dibenzofuranyl unsubstituted or substituted with a phenyl(s); a dibenzothiophenyl unsubstituted or substituted with a phenyl(s); a benzonaphthofuranyl; a benzonaphthothiophenyl, etc.

According to one embodiment of the present disclosure, L₁ to L₃, each independently, represent a single bond, a substituted or unsubstituted (C6-C25)arylene, or a substituted or unsubstituted (5- to 25-membered)heteroarylene. According to another embodiment of the present disclosure, L₁ to L₃, each independently, represent a single bond, a (C6-C18)arylene unsubstituted or substituted with a (C6-C18)aryl(s), or an unsubstituted (5- to 20-membered)heteroarylene. For example, L₁ may be a single bond, a phenylene unsubstituted or substituted with a phenyl(s); a naphthylene; a biphenylene; a pyridylene, etc., and L₂ and L₃, each independently, may be a single bond, a phenylene, a naphthylene, a dibenzofluorenylene, etc.

According to one embodiment of the present disclosure, the formula 1 may be represented by any one of the following formulas 1-11 to 1-16:

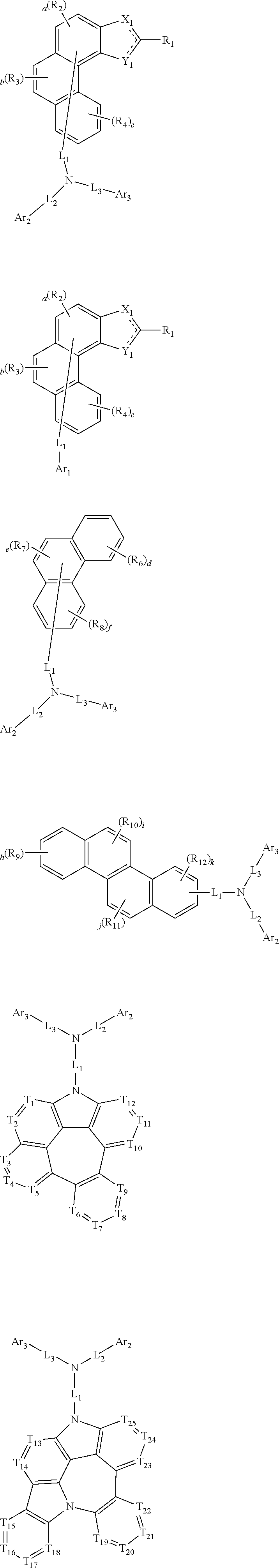

in formulas 1-11 to 1-16,
Ar₁ represents a substituted or unsubstituted (C6-C30)aryl, or a substituted or unsubstituted (3- to 30-membered)heteroaryl; and
X₁, Y₁, R₁ to R₄, R₆ to R₁₂, T₁ to T₂₅, L₁ to L₃, Ar₂, Ar₃, a to f, and h to k are as defined in formula 1.

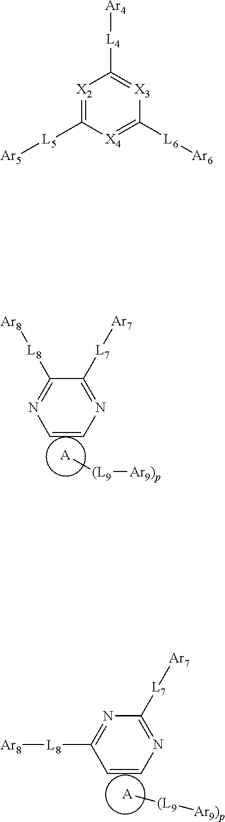

In formulas 2 to 4,
X₂ to X₄, each independently, represent CR₁₃ or N, with the proviso that at least one of X₂ to X₄ represents N;
R₁₃, each independently, represents hydrogen, deuterium, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; or may be linked to an adjacent substituent(s) to form a ring(s);
ring A represents a benzene ring or a naphthalene ring;
L₄ to L₉, each independently, represent a single bond, a substituted or unsubstituted (C6-C30)arylene, or a substituted or unsubstituted (3- to 30-membered)heteroarylene;
Ar₄ to Ar₉, each independently, represent a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; or may be linked to an adjacent substituent(s) to form a ring(s); and
p represents an integer of 0 to 6; where if p is an integer of 2 or more, each of L₉ and each of Ar₉ may be the same or different.

According to one embodiment of the present disclosure, any one of X₂ to X₄ may be N; any two of X₂ to X₄ may be N; or all of X₂ to X₄ may be N.

According to one embodiment of the present disclosure, R₁₃, each independently, represents hydrogen or deuterium; or may be linked to an adjacent substituent(s) to form a ring(s). According to another embodiment of the present disclosure, R₁₃, each independently, represents hydrogen or deuterium; or may be linked to an adjacent substituent(s) to form an unsubstituted (6- to 17-membered) ring(s). For example, R₁₃, each independently, may be hydrogen or deuterium; or may be linked to an adjacent substituent(s) to form a benzene ring, a benzonaphthofuran ring, an acenaphthylene ring, or a dihydrodimethylanthracene ring.

According to one embodiment of the present disclosure, L₄ to L₉, each independently, represent a single bond, a substituted or unsubstituted (C6-C25)arylene, or a substituted or unsubstituted (5- to 25-membered)heteroarylene.

According to another embodiment of the present disclosure, L₄ to L₉, each independently, represent a single bond, a (C6-C28)arylene unsubstituted or substituted with a (C6-C18)aryl(s), or an unsubstituted (5- to 20-membered)heteroarylene. For example, L₄ to L₉, each independently, may be a single bond, a phenylene unsubstituted or substituted with a phenyl(s), a naphthylene, a biphenylene, a phenylene-naphthylene, a naphthylene-phenylene, a phenanthrenylene, a pyridylene, a dibenzofuranylene, a naphthooxazolylene, a benzonaphthothiophenylene, etc., which may be further substituted with deuterium.

According to one embodiment of the present disclosure, Ar₄ to Ar₉, each independently, represent a substituted or unsubstituted (C1-C20)alkyl, a substituted or unsubstituted (C3-C25)cycloalkyl, a substituted or unsubstituted (C6-C30)aryl, a substituted or unsubstituted (5- to 25-membered)heteroaryl, a substituted or unsubstituted tri(C6-C25)arylsilyl, or a substituted or unsubstituted tri(C1-C20)alkylsilyl; or may be linked to an adjacent substituent(s) to form a ring(s). According to another embodiment of the present disclosure, Ar₄ to Ar₉, each independently, represent an unsubstituted (C1-C10)alkyl; an unsubstituted (C3-C18)cycloalkyl; a (C6-C30)aryl unsubstituted or substituted with at least one of deuterium, a halogen(s), a cyano(s), and a (5- to 25-membered)heteroaryl(s); a (5- to 25-membered)heteroaryl unsubstituted or substituted with deuterium, a (C6-C18)aryl(s), or a (5- to 25-membered)heteroaryl(s); an unsubstituted tri(C6-C18)arylsilyl; or an unsubstituted tri(C1-C10)alkylsilyl; or may be linked to an adjacent substituent(s) to form an unsubstituted (6- to 17-membered) ring(s). For example, Ar₄ to Ar₉, each independently, may be a tert-butyl; a cyclohexyl; a phenyl unsubstituted or substituted with a fluoro(s) or a cyano(s); a naphthyl unsubstituted or substituted with a dibenzofuranyl(s) or a benzonaphthofuranyl(s); a naphthylphenyl; a phenylnaphthyl; a biphenyl; a phenanthrenyl; a dihydrophenanthrenyl substituted with a methyl(s); an anthracenyl; a terphenyl; a triphenylenyl; a dimethylfluorenyl; a phenylfluorenyl; a diphenylfluorenyl unsubstituted or substituted with a phenyl(s); a dimethylbenzofluorenyl; a diphenylbenzofluorenyl; a spirobifluorenyl; a spiro[benzofluoren-fluoren]yl; a terphenyl; a chrysenyl unsubstituted or substituted with a phenyl(s); a (C22)aryl; a carbazolyl unsubstituted or substituted with a phenyl(s); a benzothiophenyl; a dibenzofuranyl unsubstituted or substituted with a phenyl(s) or a biphenyl(s); a dibenzothiophenyl; a substituted or unsubstituted benzonaphthothiophenyl; a substituted or unsubstituted benzonaphthofuranyl; a benzophenanthrofuranyl; a naphthooxazolyl substituted with a phenyl(s); a phenanthrooxazolyl substituted with a phenyl(s); a phenoxazinyl; a triphenylsilyl; or a trimethylsilyl; or may be linked to an adjacent substituent(s) to form a benzene ring, a benzonaphthofuran ring, an acenaphthylene ring, or a dihydrodimethylanthracene ring, which may be further substituted with deuterium. The substituent(s) of the substituted benzonaphthothiophenyl and the substituted benzonaphthofuranyl, each independently, may be at least one selected from the group consisting of deuterium, a phenyl, a naphthyl, a biphenyl, a dimethylfluorenyl, a dibenzofuranyl, a dibenzothiophenyl, and a phenanthrenyl.

According to one embodiment of the present disclosure, p represents an integer of 0 to 4. According to another embodiment of the present disclosure, p may be 0 or 1.

According to one embodiment of the present disclosure, at least one of Ar₄ to Ar₉ is selected from the group consisting of the following formulas a-1 to a-7:

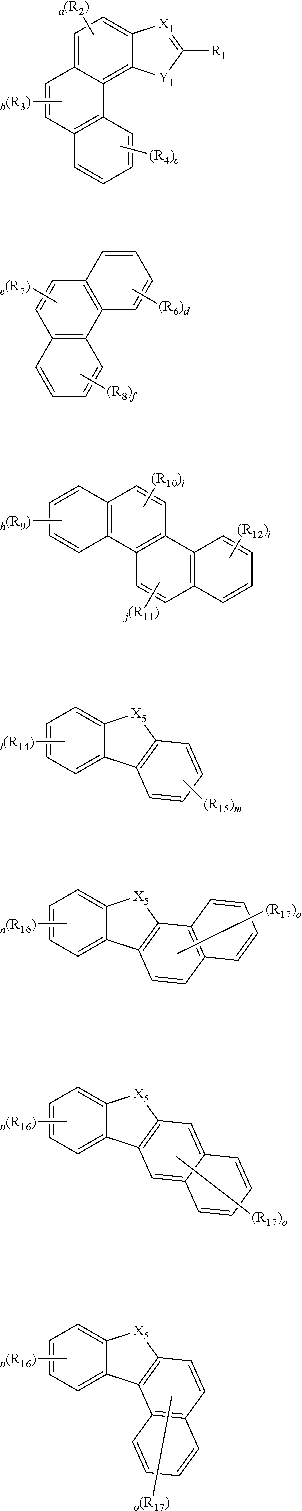

in formulas a-1 to a-7,
X₅, each independently, represents O, S, or CR₁₈R₁₉;
R₁₈ and R₁₉, each independently, represent a substituted or unsubstituted (C1-C30)alkyl, a substituted or unsubstituted (C3-C30)cycloalkyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; or R₁₈ and R₁₉ may be linked to each other to form a spiro ring;
R₁₄ and R₁₅, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
l to n, each independently, represent an integer of 1 to 4; o represents an integer of 1 to 6; where if each of l to o is an integer of 2 or more, each of R₁₄ to each of R₁₇ may be the same or different;
R₆ to R₈, R₁₆, and R₁₇, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent(s) to form a ring(s); and
X₁, Y₁, R₁ to R₄, R₉ to R₁₂, a to f, and h to k are as defined in formula 2.

According to one embodiment of the present disclosure, R₁₈ and R₁₉, each independently, represent a substituted or unsubstituted (C1-C20)alkyl, a substituted or unsubstituted (C6-C25)aryl, or a substituted or unsubstituted (3- to 30-membered)heteroaryl; or R₁₈ and R₁₉ may be linked to each other to form a spiro ring. According to another embodiment of the present disclosure, R₁₈ and R₁₉, each independently, represent an unsubstituted (C1-C10)alkyl, or an unsubstituted (C6-C18)aryl; or R₁₈ and R₁₉ may be linked to each other to form a spiro ring. For example, R₁₈ and R₁₉, each independently, may be a methyl or a phenyl; or R₁₈ and R₁₉ may be linked to each other to form a spiro fluorene ring.

According to one embodiment of the present disclosure, R₁₄ and R₁₅, each independently, represent hydrogen, deuterium, a substituted or unsubstituted (C6-C25)aryl, or a substituted or unsubstituted (5- to 25-membered)heteroaryl. According to another embodiment of the present disclosure, R₁₄ and R₁₅, each independently, represent hydrogen, deuterium, a (C6-C18)aryl unsubstituted or substituted with a (C1-C10)alkyl(s), or an unsubstituted (5- to 20-membered)heteroaryl. For example, R₁₄ and R₁₅, each independently, represent hydrogen, deuterium, a phenyl, a naphthyl, a biphenyl, a phenanthrenyl, a dimethylfluorenyl, a dibenzofuranyl, a dibenzothiophenyl, etc.

According to one embodiment of the present disclosure, R₁₆ and R₁₇, each independently, represent hydrogen, deuterium, a substituted or unsubstituted (C6-C25)aryl, or a substituted or unsubstituted (5- to 25-membered)heteroaryl; or may be linked to an adjacent substituent(s) to form a ring(s). According to another embodiment of the present disclosure, R₁₆ and R₁₇, each independently, represent hydrogen, deuterium, a (C6-C18)aryl unsubstituted or substituted with a (C1-C10)alkyl(s), or an unsubstituted (5- to 20-membered)heteroaryl; or may be linked to an adjacent substituent(s) to form a ring(s). For example, R₁₆ and R₁₇, each independently, may be hydrogen, deuterium, a phenyl, a naphthyl, a biphenyl, a phenanthrenyl, a dimethylfluorenyl, a dibenzofuranyl, a dibenzothiophenyl, etc.; or may be linked to an adjacent substituent to form a benzene ring.

According to one embodiment of the present disclosure, R₆ to R₈, each independently, represent hydrogen or a substituted or unsubstituted (C6-C25)aryl; or may be linked to an adjacent substituent(s) to form a ring(s). According to another embodiment of the present disclosure, R₆ to R₈, each independently, represent hydrogen or an unsubstituted (C6-C18)aryl; or may be linked to an adjacent substituent(s) to form a substituted or unsubstituted (C6-C18) aromatic ring(s). For example, R₆ to R₈, each independently, may be hydrogen or a phenyl; or may be linked to an adjacent substituent to form a benzene ring unsubstituted or substituted with a phenyl(s).

The compound represented by formula 1 may be at least one selected from the group consisting of the following compounds, but is not limited thereto.

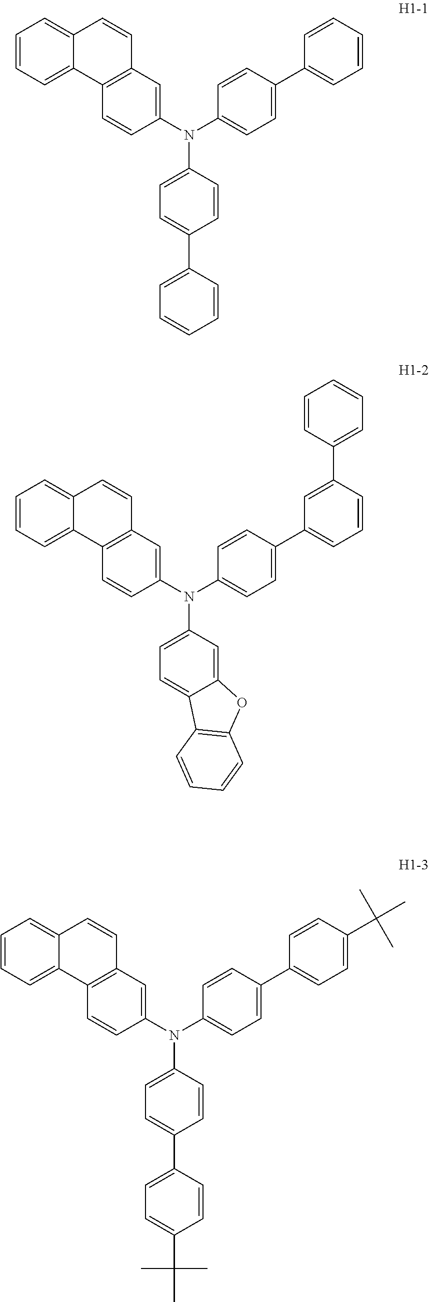 [SMILES: c1ccc(-c2ccc(N(c3ccc(-c4ccccc4)cc3)c3ccc4c(ccc5ccccc54)c3)cc2)cc1 ; c1ccc(-c2cccc(-c3ccc(N(c4ccc5c(ccc6ccccc65)c4)c4ccc5c(c4)oc4ccccc45)cc3)c2)cc1 ; CC(C)(C)c1ccc(-c2ccc(N(c3ccc(-c4ccc(C(C)(C)C)cc4)cc3)c3ccc4c(ccc5ccccc54)c3)cc2)cc1]

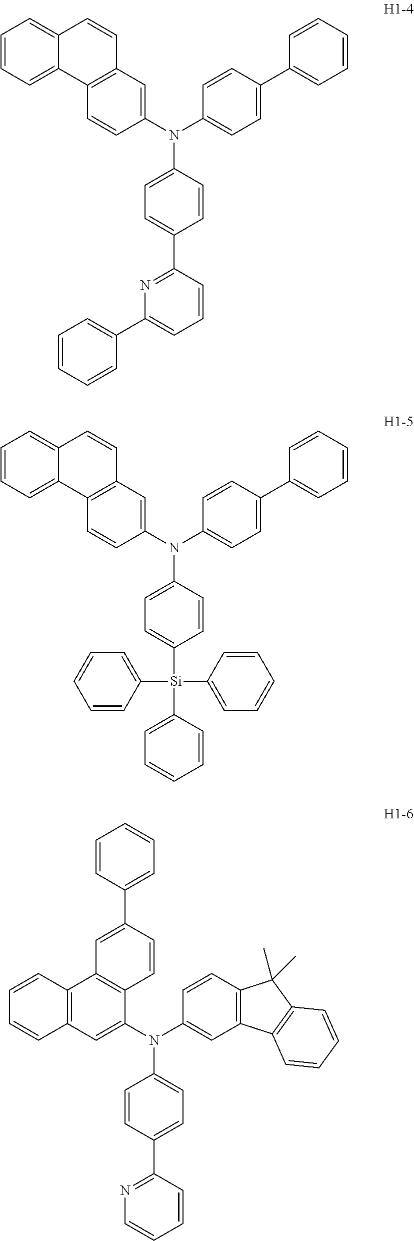

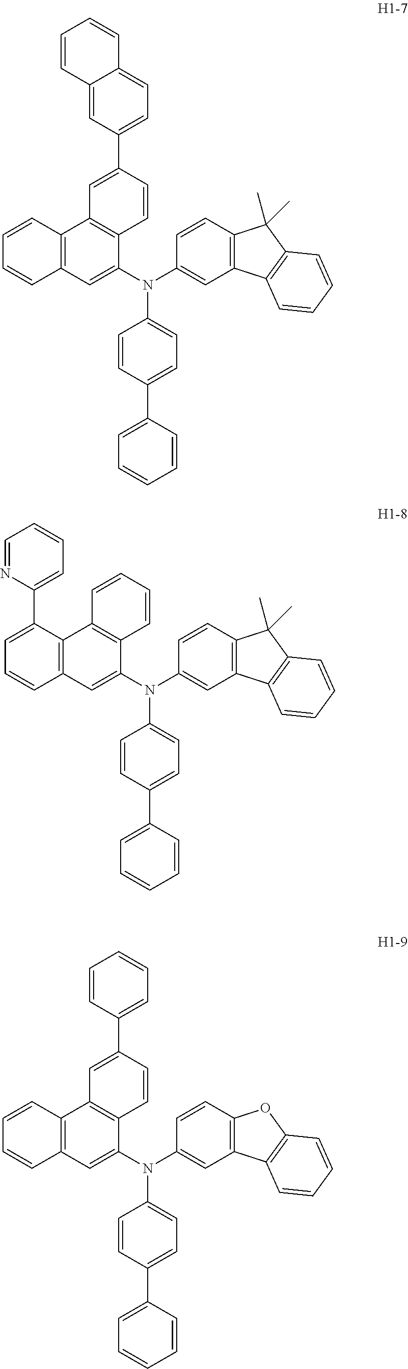 [SMILES: CC1(C)c2ccccc2-c2cc(N(c3ccc(-c4ccccc4)cc3)c3cc4ccccc4c4cc(-c5ccc6ccccc6c5)ccc34)ccc21 ; CC1(C)c2ccccc2-c2cc(N(c3ccc(-c4ccccc4)cc3)c3cc4cccc(-c5ccccn5)c4c4ccccc34)ccc21 ; c1ccc(-c2ccc(N(c3ccc4oc5ccccc5c4c3)c3cc4ccccc4c4cc(-c5ccccc5)ccc34)cc2)cc1]

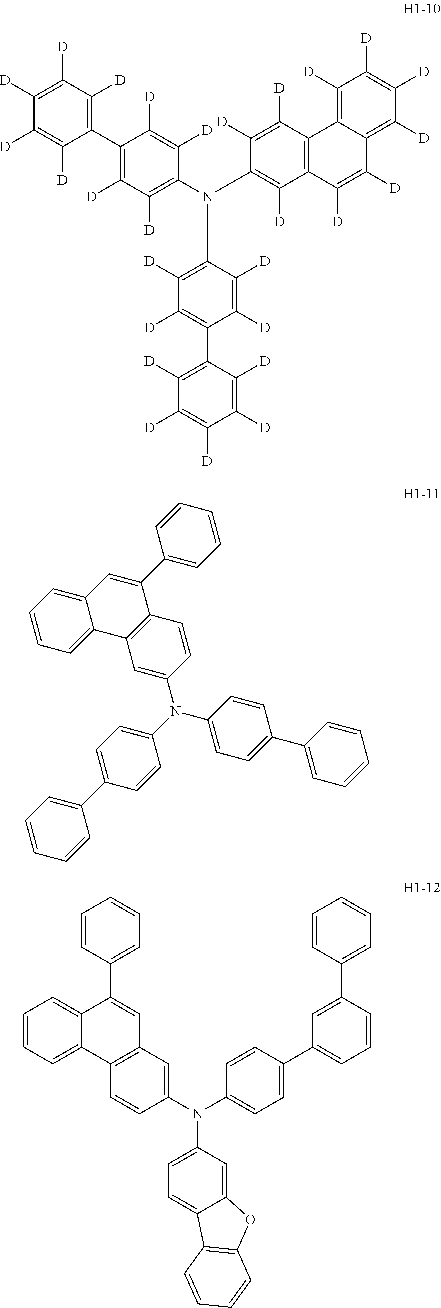 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c([2H])c([2H])c([2H])c([2H])c34)c([2H])c2[2H])c([2H])c1[2H] ; c1ccc(-c2ccc(N(c3ccc(-c4ccccc4)cc3)c3ccc4c(-c5ccccc5)cc5ccccc5c4c3)cc2)cc1 ; c1ccc(-c2cccc(-c3ccc(N(c4ccc5c(c4)cc(-c4ccccc4)c4ccccc45)c4ccc5c(c4)oc4ccccc45)cc3)c2)cc1]

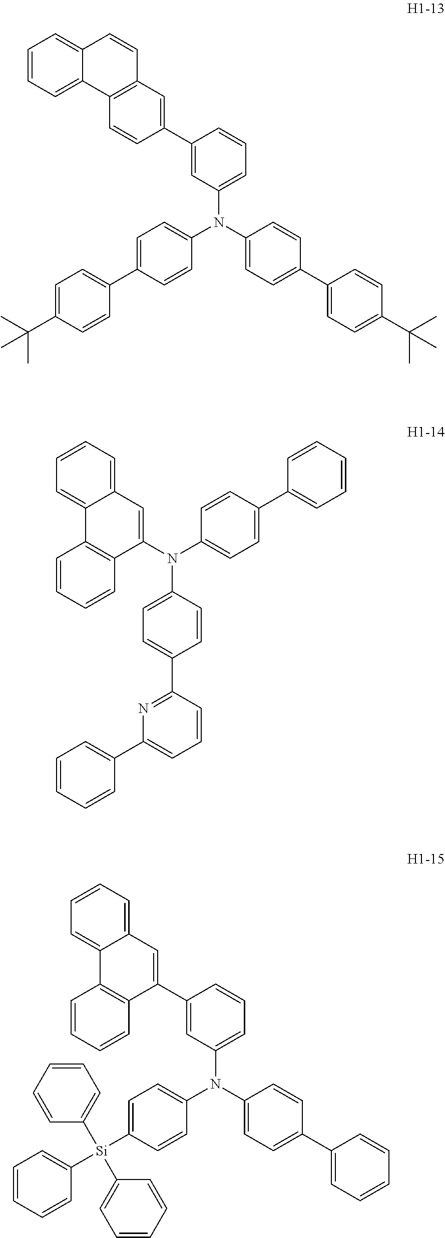 [SMILES: CC(C)(C)c1ccc(-c2ccc(N(c3ccc(-c4ccc(C(C)(C)C)cc4)cc3)c3cccc(-c4ccc5c(ccc6ccccc65)c4)c3)cc2)cc1 ; c1ccc(-c2ccc(N(c3ccc(-c4cccc(-c5ccccc5)n4)cc3)c3cc4ccccc4c4ccccc34)cc2)cc1 ; c1ccc(-c2ccc(N(c3ccc([Si](c4ccccc4)(c4ccccc4)c4ccccc4)cc3)c3cccc(-c4cc5ccccc5c5ccccc45)c3)cc2)cc1]

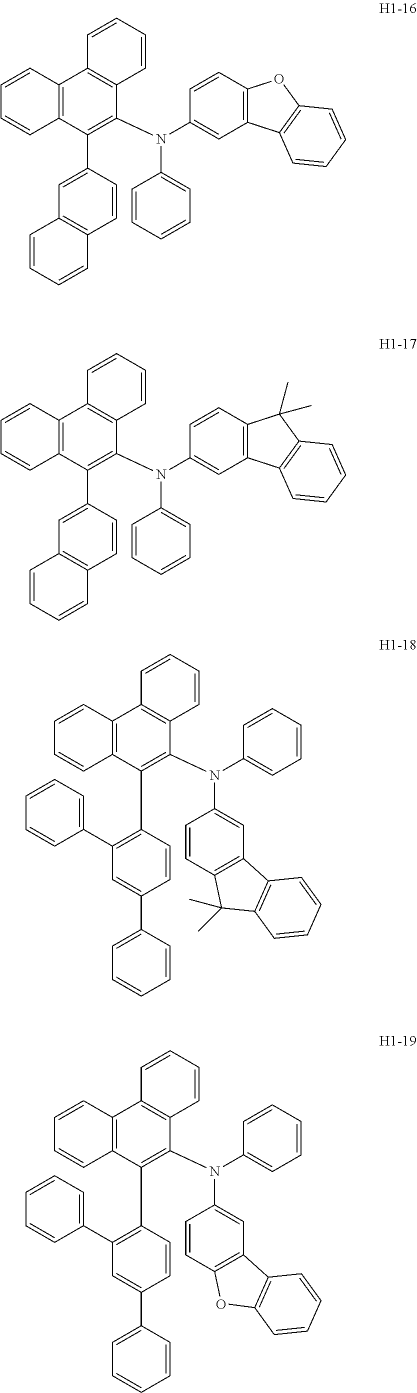 [SMILES: c1ccc(N(c2ccc3oc4ccccc4c3c2)c2c(-c3ccc4ccccc4c3)c3ccccc3c3ccccc23)cc1 ; CC1(C)c2ccccc2-c2cc(N(c3ccccc3)c3c(-c4ccc5ccccc5c4)c4ccccc4c4ccccc34)ccc21 ; CC1(C)c2ccccc2-c2cc(N(c3ccccc3)c3c(-c4ccc(-c5ccccc5)cc4-c4ccccc4)c4ccccc4c4ccccc34)ccc21 ; c1ccc(-c2ccc(-c3c(N(c4ccccc4)c4ccc5oc6ccccc6c5c4)c4ccccc4c4ccccc34)c(-c3ccccc3)c2)cc1]

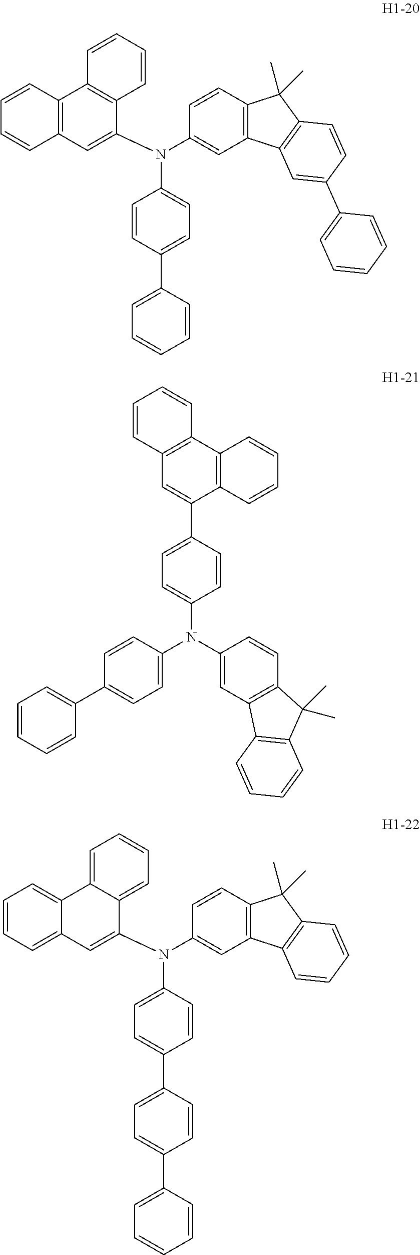

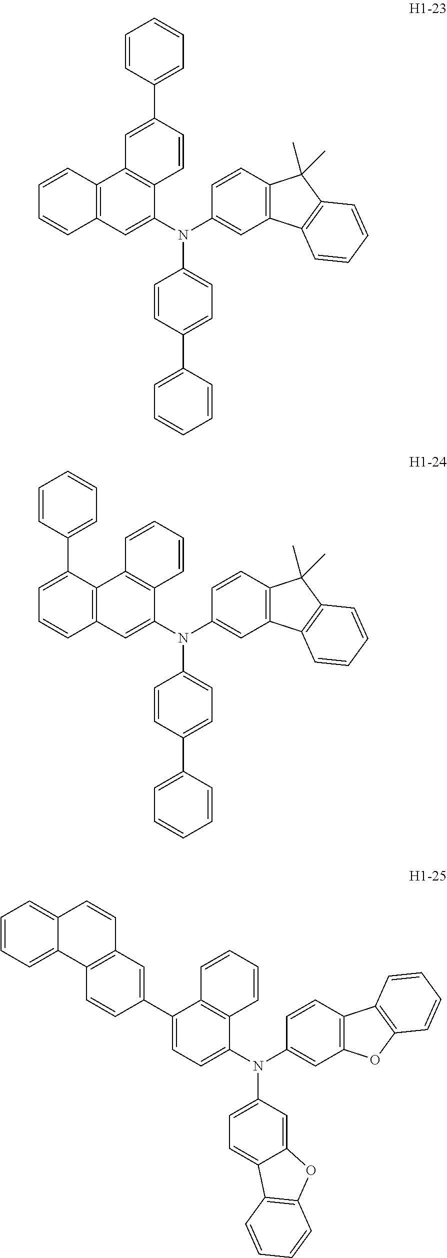 [SMILES: CC1(C)c2ccccc2-c2cc(N(c3ccc(-c4ccccc4)cc3)c3cc4ccccc4c4cc(-c5ccccc5)ccc34)ccc21 ; CC1(C)c2ccccc2-c2cc(N(c3ccc(-c4ccccc4)cc3)c3cc4cccc(-c5ccccc5)c4c4ccccc34)ccc21 ; c1ccc2c(c1)ccc1cc(-c3ccc(N(c4ccc5c(c4)oc4ccccc45)c4ccc5c(c4)oc4ccccc45)c4ccccc34)ccc12]

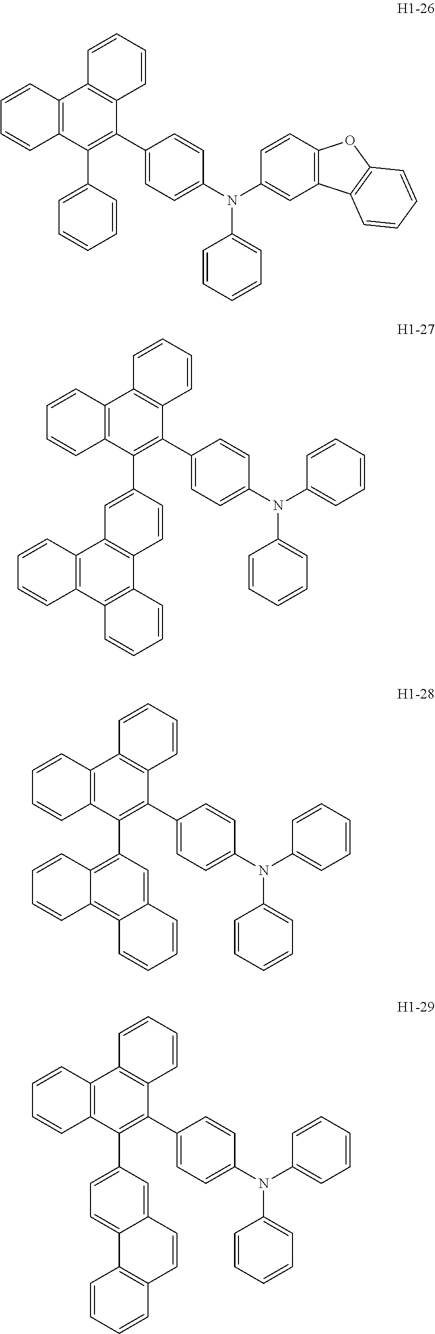

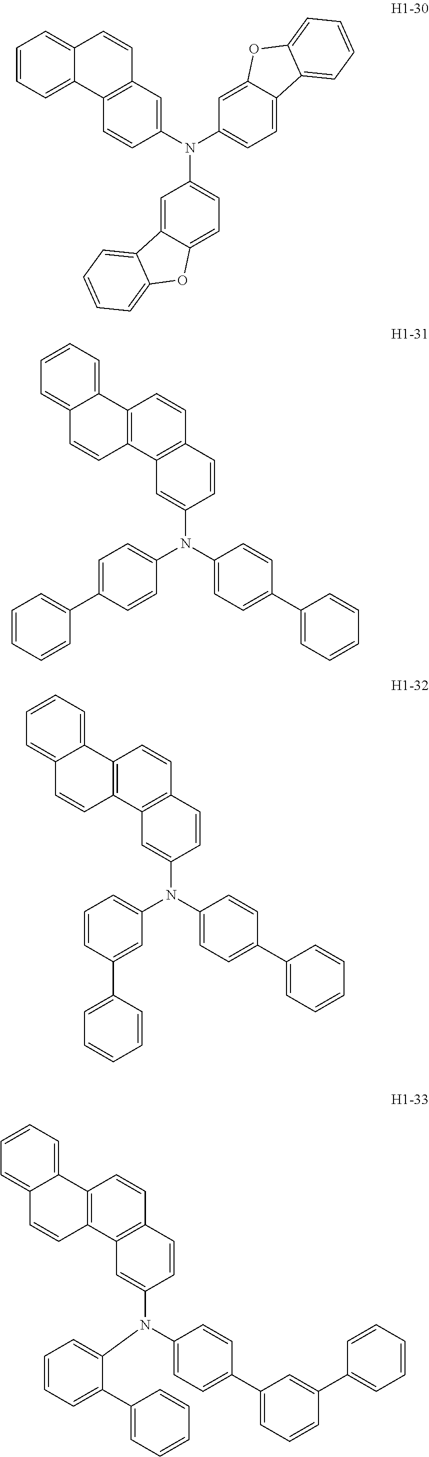

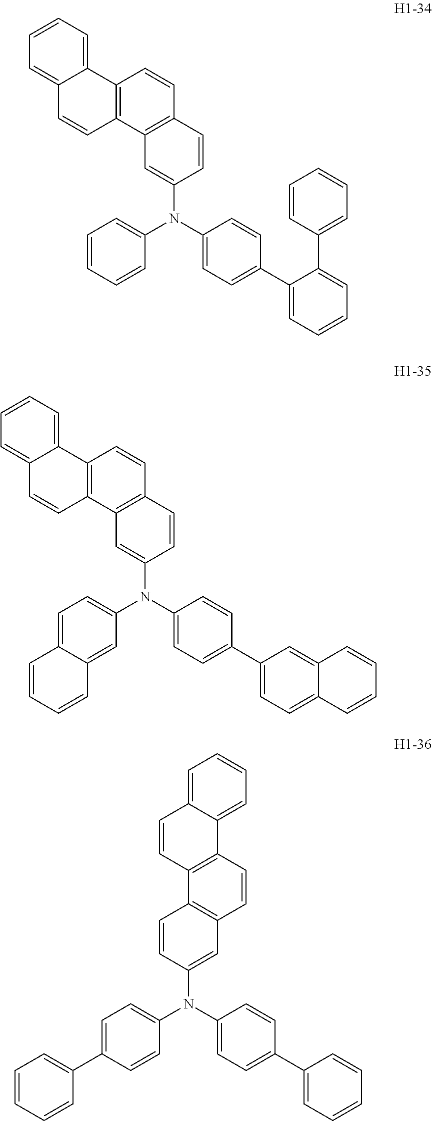 [SMILES: c1ccc(-c2ccccc2-c2ccc(N(c3ccccc3)c3ccc4ccc5c6ccccc6ccc5c4c3)cc2)cc1 ; c1ccc2cc(-c3ccc(N(c4ccc5ccccc5c4)c4ccc5ccc6c7ccccc7ccc6c5c4)cc3)ccc2c1 ; c1ccc(-c2ccc(N(c3ccc(-c4ccccc4)cc3)c3ccc4c(ccc5c6ccccc6ccc45)c3)cc2)cc1]

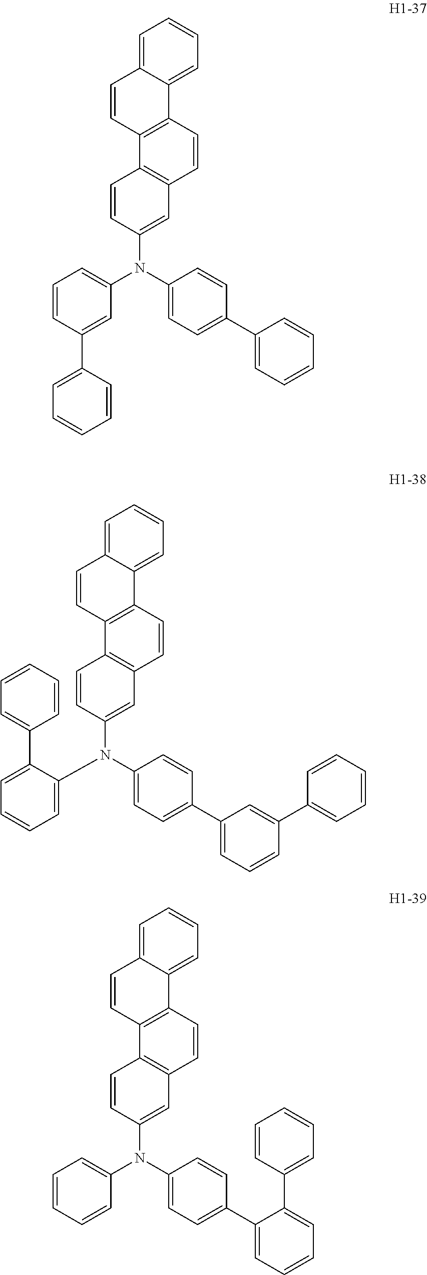 [SMILES: c1ccc(-c2ccc(N(c3cccc(-c4ccccc4)c3)c3ccc4c(ccc5c6ccccc6ccc45)c3)cc2)cc1 ; c1ccc(-c2cccc(-c3ccc(N(c4ccc5c(ccc6c7ccccc7ccc56)c4)c4ccccc4-c4ccccc4)cc3)c2)cc1 ; c1ccc(-c2ccccc2-c2ccc(N(c3ccccc3)c3ccc4c(ccc5c6ccccc6ccc45)c3)cc2)cc1]

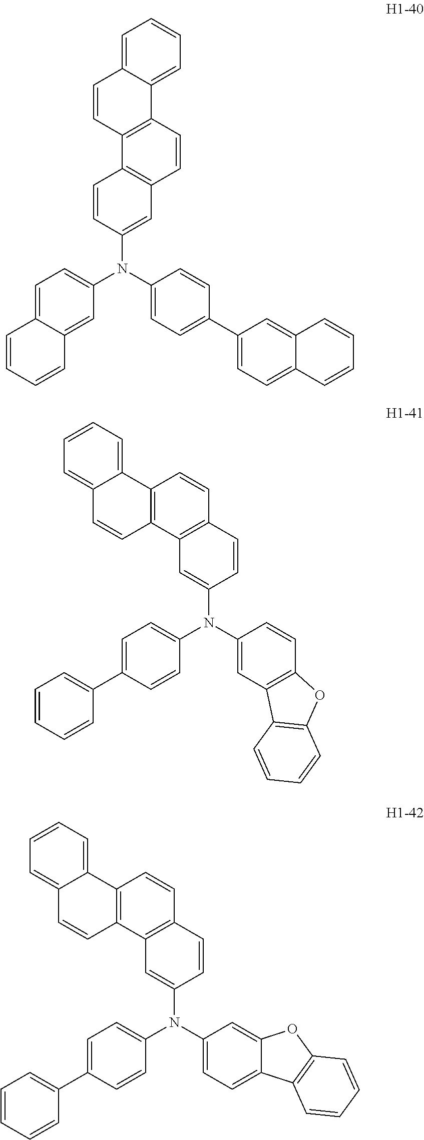 [SMILES: c1ccc2cc(-c3ccc(N(c4ccc5ccccc5c4)c4ccc5c(ccc6c7ccccc7ccc56)c4)cc3)ccc2c1 ; c1ccc(-c2ccc(N(c3ccc4ccc5c6ccccc6ccc5c4c3)c3ccc4oc5ccccc5c4c3)cc2)cc1 ; c1ccc(-c2ccc(N(c3ccc4c(c3)oc3ccccc34)c3ccc4ccc5c6ccccc6ccc5c4c3)cc2)cc1]

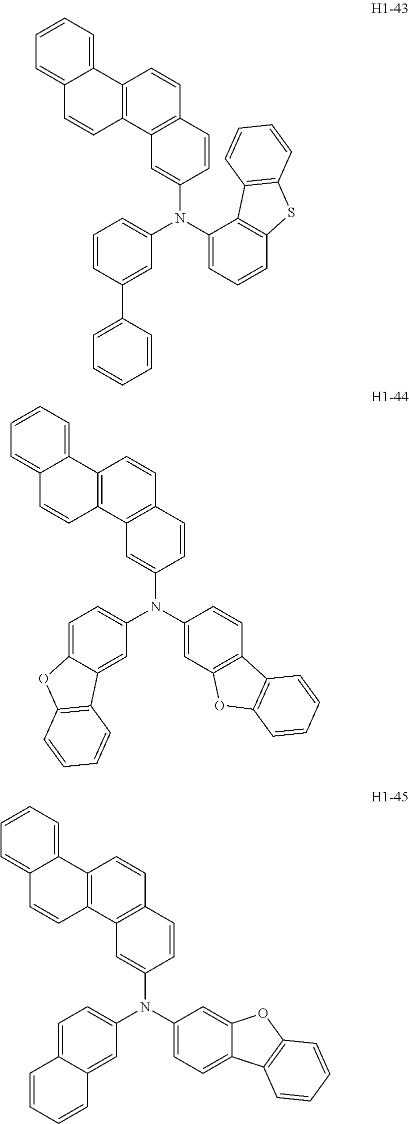 [SMILES: c1ccc(-c2cccc(N(c3ccc4ccc5c6ccccc6ccc5c4c3)c3cccc4sc5ccccc5c34)c2)cc1 ; c1ccc2c(c1)ccc1c3cc(N(c4ccc5c(c4)oc4ccccc45)c4ccc5oc6ccccc6c5c4)ccc3ccc21 ; c1ccc2cc(N(c3ccc4c(c3)oc3ccccc34)c3ccc4ccc5c6ccccc6ccc5c4c3)ccc2c1]

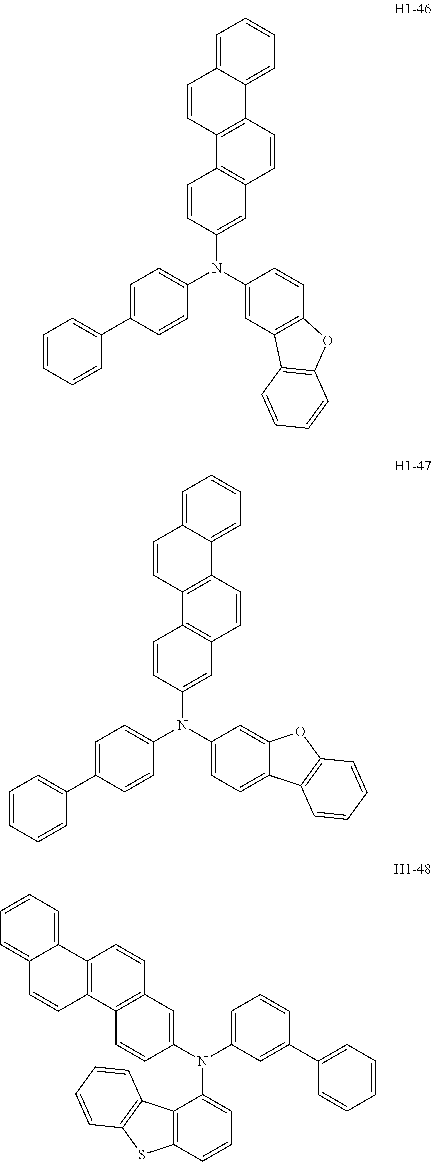 [SMILES: c1ccc(-c2ccc(N(c3ccc4c(ccc5c6ccccc6ccc45)c3)c3ccc4oc5ccccc5c4c3)cc2)cc1 ; c1ccc(-c2ccc(N(c3ccc4c(ccc5c6ccccc6ccc45)c3)c3ccc4c(c3)oc3ccccc34)cc2)cc1 ; c1ccc(-c2cccc(N(c3ccc4c(ccc5c6ccccc6ccc45)c3)c3cccc4sc5ccccc5c34)c2)cc1]

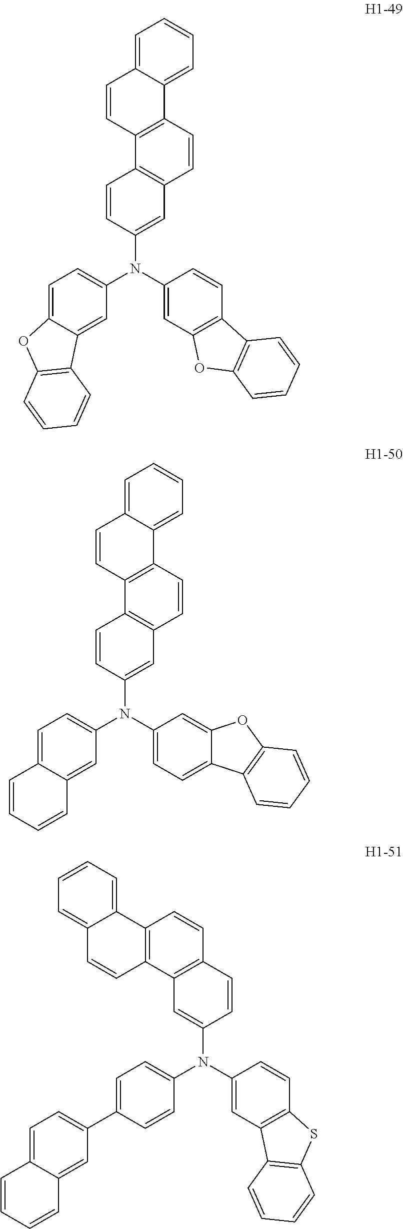 [SMILES: c1ccc2c(c1)ccc1c3ccc(N(c4ccc5c(c4)oc4ccccc45)c4ccc5oc6ccccc6c5c4)cc3ccc21 ; c1ccc2cc(N(c3ccc4c(ccc5c6ccccc6ccc45)c3)c3ccc4c(c3)oc3ccccc34)ccc2c1 ; c1ccc2cc(-c3ccc(N(c4ccc5ccc6c7ccccc7ccc6c5c4)c4ccc5sc6ccccc6c5c4)cc3)ccc2c1]

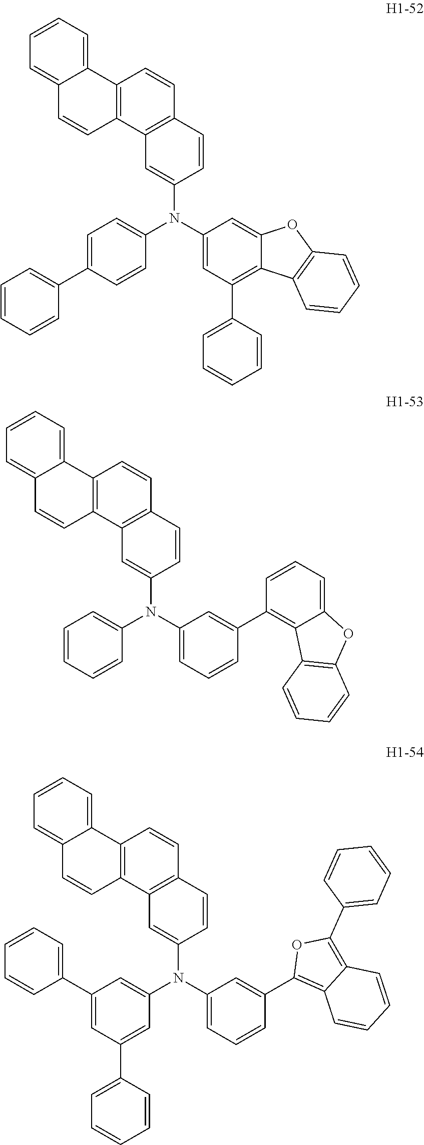 [SMILES: c1ccc(-c2ccc(N(c3cc(-c4ccccc4)c4c(c3)oc3ccccc34)c3ccc4ccc5c6ccccc6ccc5c4c3)cc2)cc1 ; c1ccc(N(c2cccc(-c3cccc4oc5ccccc5c34)c2)c2ccc3ccc4c5ccccc5ccc4c3c2)cc1 ; c1ccc(-c2cc(-c3ccccc3)cc(N(c3cccc(-c4oc(-c5ccccc5)c5ccccc45)c3)c3ccc4ccc5c6ccccc6ccc5c4c3)c2)cc1]

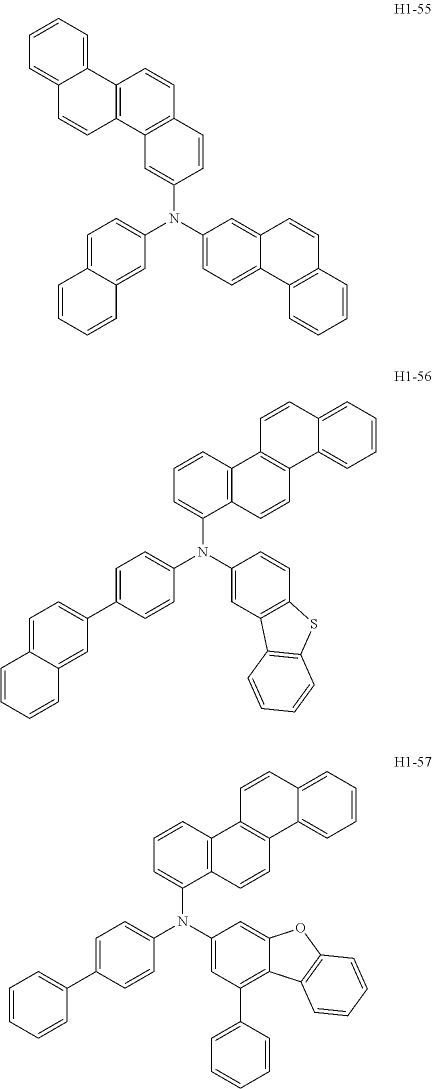 [SMILES: c1ccc2cc(N(c3ccc4c(ccc5ccccc54)c3)c3ccc4ccc5c6ccccc6ccc5c4c3)ccc2c1 ; c1ccc2cc(-c3ccc(N(c4ccc5sc6ccccc6c5c4)c4cccc5c4ccc4c6ccccc6ccc54)cc3)ccc2c1 ; c1ccc(-c2ccc(N(c3cc(-c4ccccc4)c4c(c3)oc3ccccc34)c3cccc4c3ccc3c5ccccc5ccc43)cc2)cc1]

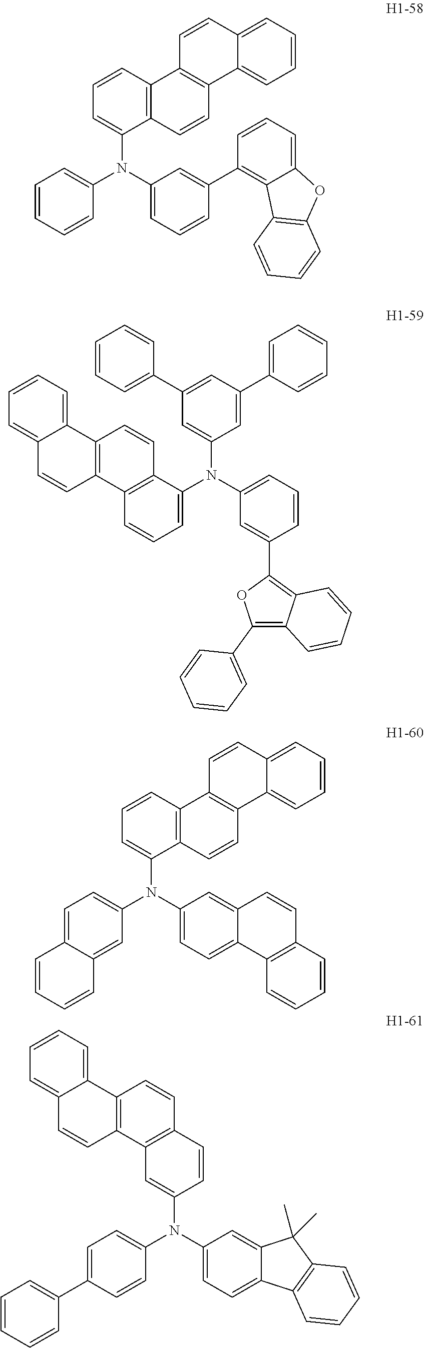

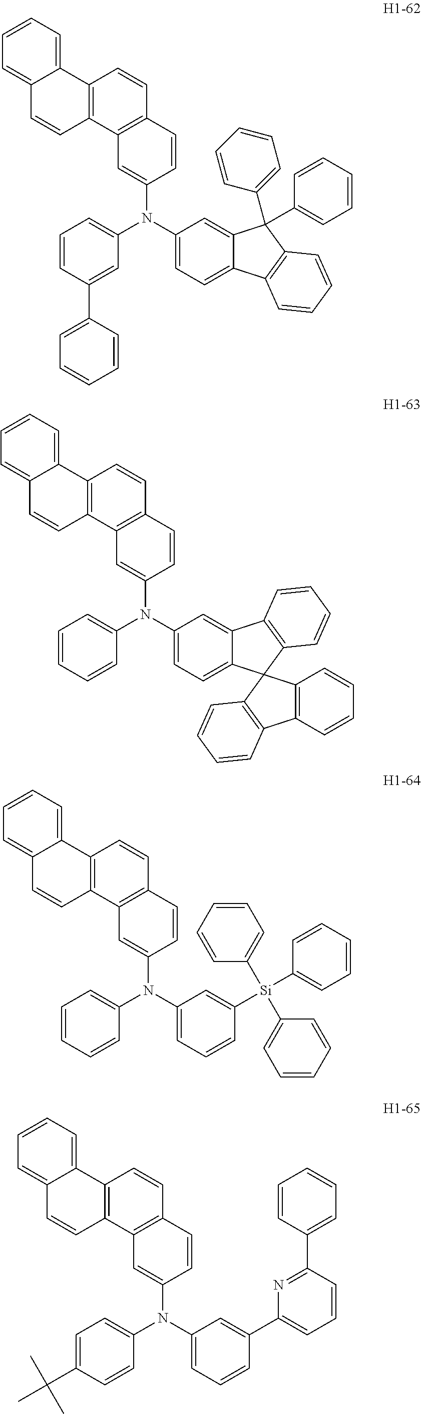 [SMILES: c1ccc(-c2cccc(N(c3ccc4c(c3)C(c3ccccc3)(c3ccccc3)c3ccccc3-4)c3ccc4ccc5c6ccccc6ccc5c4c3)c2)cc1 ; c1ccc(N(c2ccc3c(c2)-c2ccccc2C32c3ccccc3-c3ccccc32)c2ccc3ccc4c5ccccc5ccc4c3c2)cc1 ; c1ccc(N(c2cccc([Si](c3ccccc3)(c3ccccc3)c3ccccc3)c2)c2ccc3ccc4c5ccccc5ccc4c3c2)cc1 ; CC(C)(C)c1ccc(N(c2cccc(-c3cccc(-c4ccccc4)n3)c2)c2ccc3ccc4c5ccccc5ccc4c3c2)cc1]

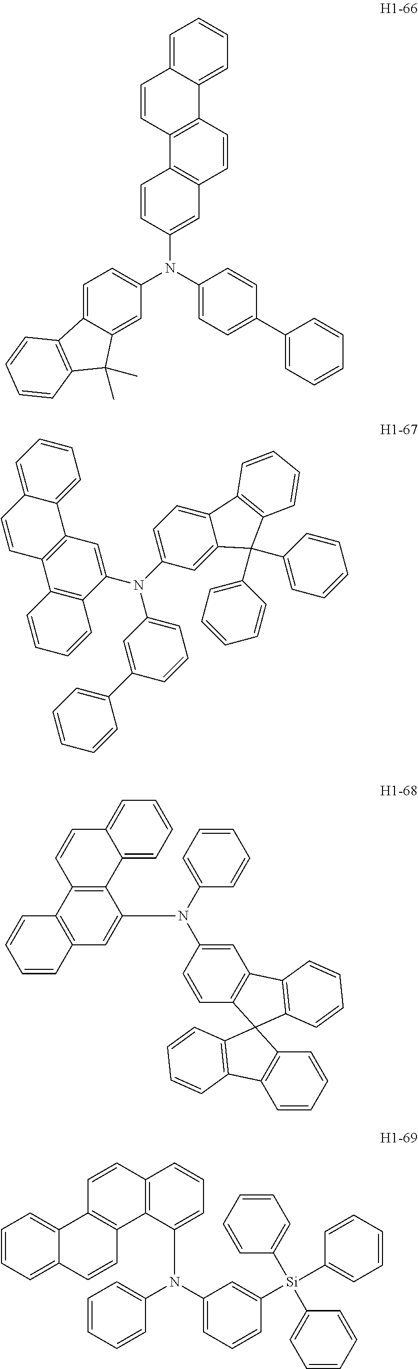

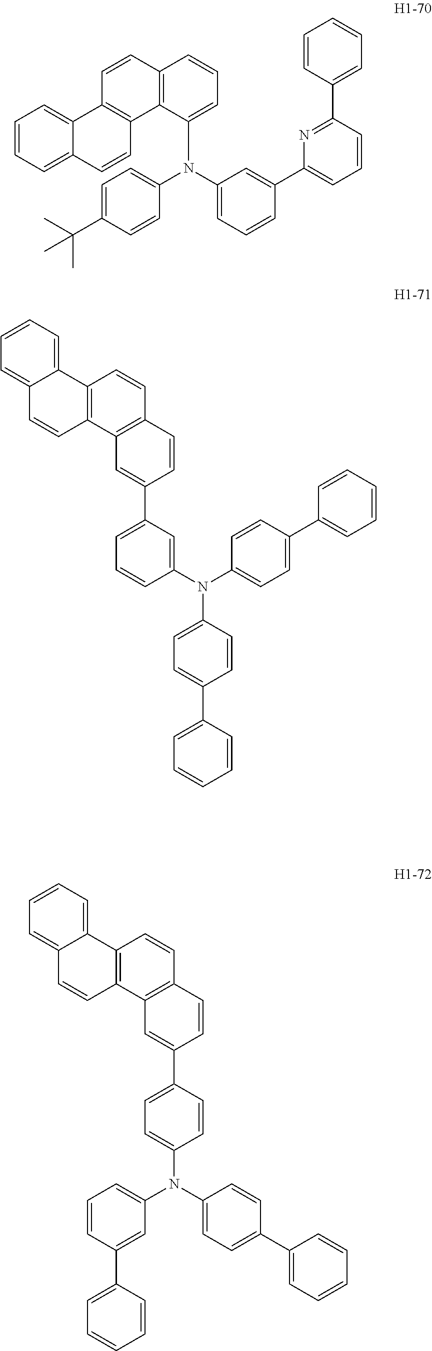 [SMILES: CC(C)(C)c1ccc(N(c2cccc(-c3cccc(-c4ccccc4)n3)c2)c2cccc3ccc4c5ccccc5ccc4c23)cc1 ; c1ccc(-c2ccc(N(c3ccc(-c4ccccc4)cc3)c3cccc(-c4ccc5ccc6c7ccccc7ccc6c5c4)c3)cc2)cc1 ; c1ccc(-c2ccc(N(c3ccc(-c4ccc5ccc6c7ccccc7ccc6c5c4)cc3)c3cccc(-c4ccccc4)c3)cc2)cc1]

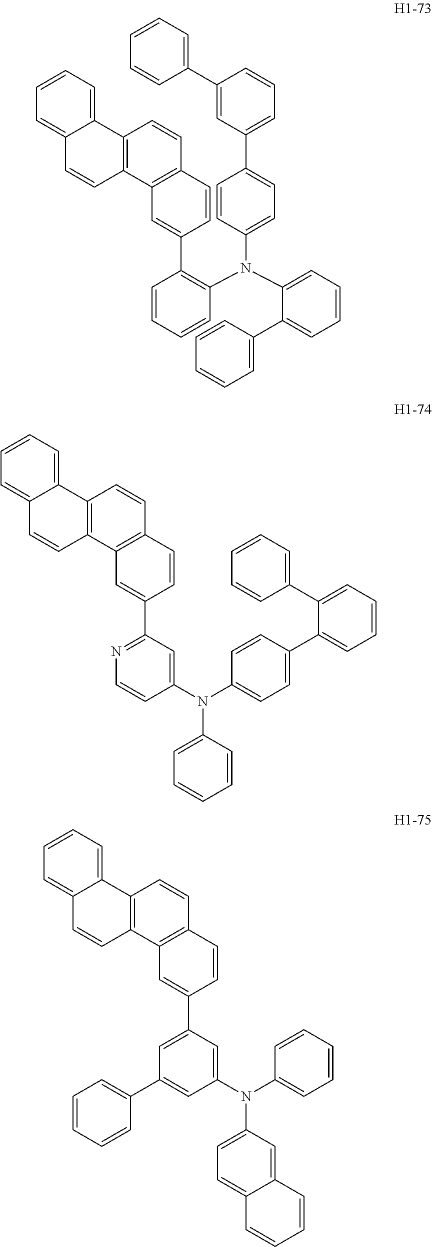 [SMILES: c1ccc(-c2cccc(-c3ccc(N(c4ccccc4-c4ccccc4)c4ccccc4-c4ccc5ccc6c7ccccc7ccc6c5c4)cc3)c2)cc1 ; c1ccc(-c2ccccc2-c2ccc(N(c3ccccc3)c3ccnc(-c4ccc5ccc6c7ccccc7ccc6c5c4)c3)cc2)cc1 ; c1ccc(-c2cc(-c3ccc4ccc5c6ccccc6ccc5c4c3)cc(N(c3ccccc3)c3ccc4ccccc4c3)c2)cc1]

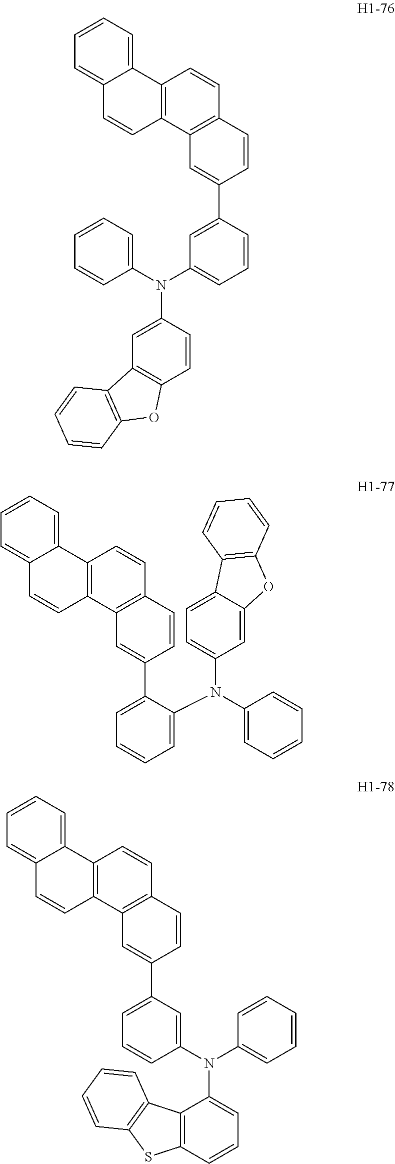 [SMILES: c1ccc(N(c2cccc(-c3ccc4ccc5c6ccccc6ccc5c4c3)c2)c2ccc3oc4ccccc4c3c2)cc1 ; c1ccc(N(c2ccc3c(c2)oc2ccccc23)c2ccccc2-c2ccc3ccc4c5ccccc5ccc4c3c2)cc1 ; c1ccc(N(c2cccc(-c3ccc4ccc5c6ccccc6ccc5c4c3)c2)c2cccc3sc4ccccc4c23)cc1]

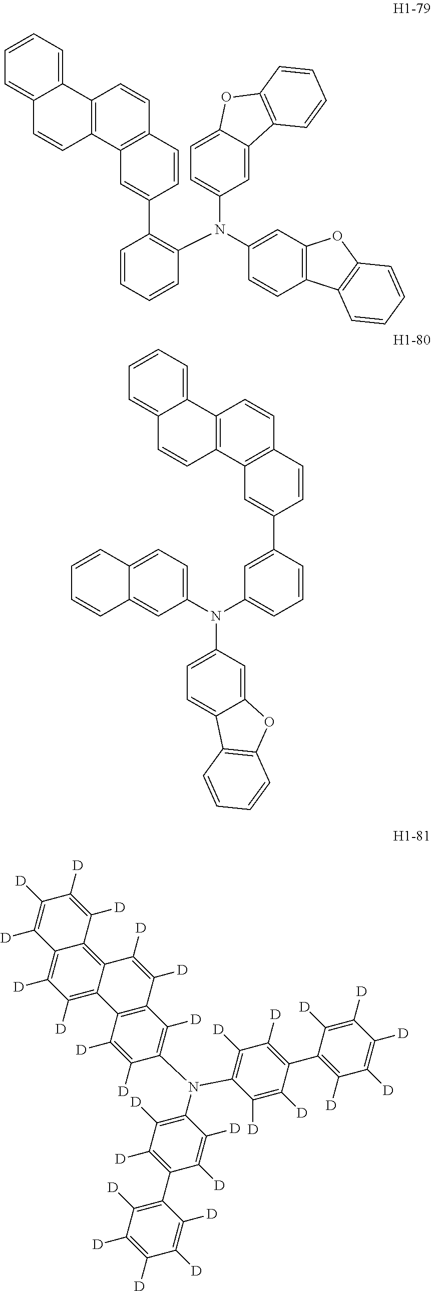 [SMILES: c1ccc(N(c2ccc3c(c2)oc2ccccc23)c2ccc3oc4ccccc4c3c2)c(-c2ccc3ccc4c5ccccc5ccc4c3c2)c1 ; c1cc(-c2ccc3ccc4c5ccccc5ccc4c3c2)cc(N(c2ccc3ccccc3c2)c2ccc3c(c2)oc2ccccc23)c1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c5c([2H])c([2H])c([2H])c([2H])c5c([2H])c([2H])c43)c([2H])c2[2H])c([2H])c1[2H]]

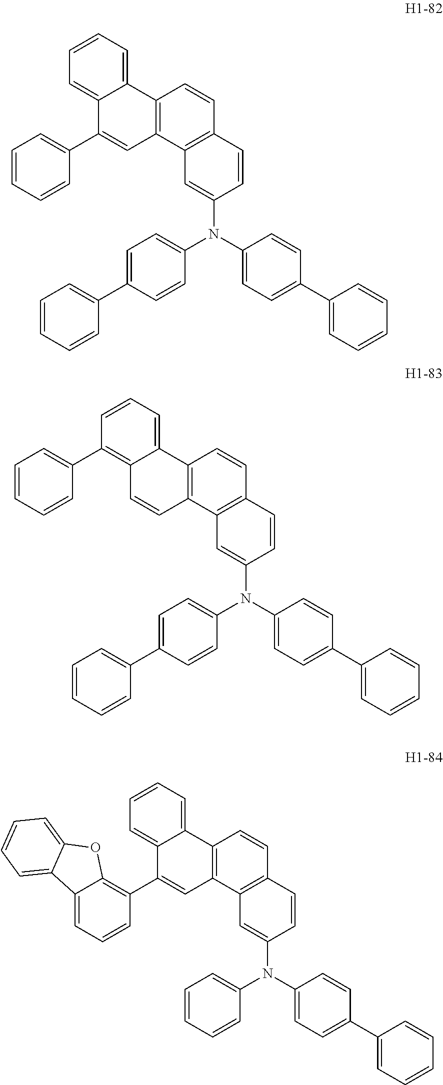 [SMILES: c1ccc(-c2ccc(N(c3ccc(-c4ccccc4)cc3)c3ccc4ccc5c6ccccc6c(-c6ccccc6)cc5c4c3)cc2)cc1 ; c1ccc(-c2ccc(N(c3ccc(-c4ccccc4)cc3)c3ccc4ccc5c6cccc(-c7ccccc7)c6ccc5c4c3)cc2)cc1 ; c1ccc(-c2ccc(N(c3ccccc3)c3ccc4ccc5c6ccccc6c(-c6cccc7c6oc6ccccc67)cc5c4c3)cc2)cc1]

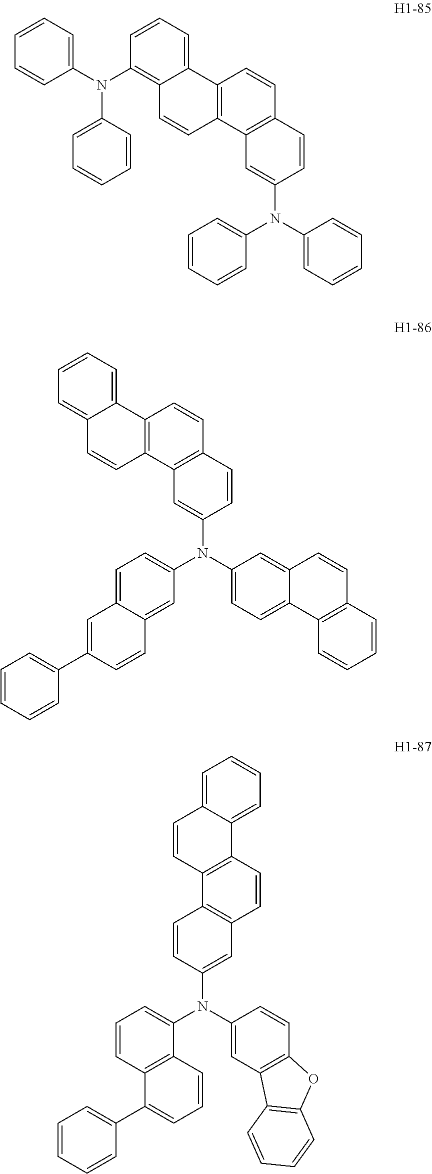 [SMILES: c1ccc(N(c2ccccc2)c2ccc3ccc4c5cccc(N(c6ccccc6)c6ccccc6)c5ccc4c3c2)cc1 ; c1ccc(-c2ccc3cc(N(c4ccc5c(ccc6ccccc65)c4)c4ccc5ccc6c7ccccc7ccc6c5c4)ccc3c2)cc1 ; c1ccc(-c2cccc3c(N(c4ccc5c(ccc6c7ccccc7ccc56)c4)c4ccc5oc6ccccc6c5c4)cccc23)cc1]

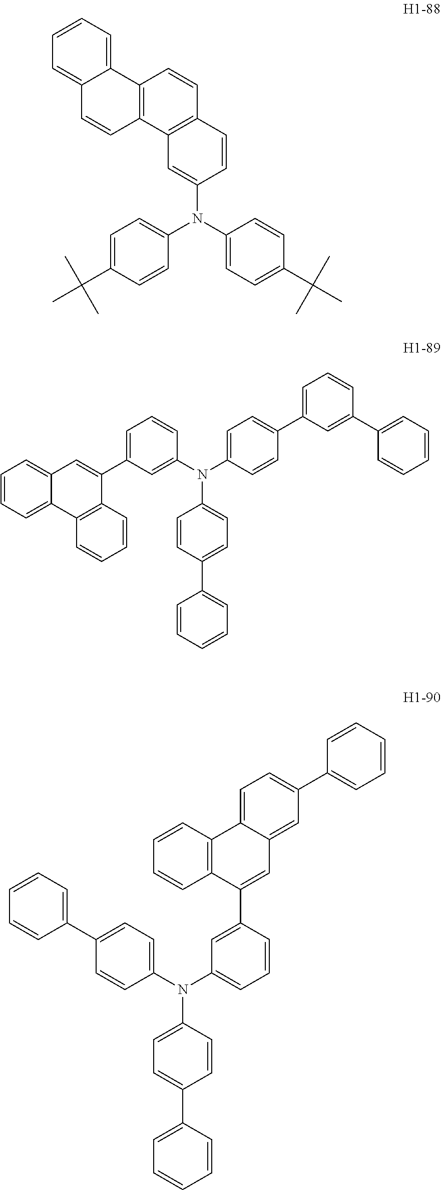 [SMILES: CC(C)(C)c1ccc(N(c2ccc(C(C)(C)C)cc2)c2ccc3ccc4c5ccccc5ccc4c3c2)cc1 ; c1ccc(-c2ccc(N(c3ccc(-c4cccc(-c5ccccc5)c4)cc3)c3cccc(-c4cc5ccccc5c5ccccc45)c3)cc2)cc1 ; c1ccc(-c2ccc(N(c3ccc(-c4ccccc4)cc3)c3cccc(-c4cc5cc(-c6ccccc6)ccc5c5ccccc45)c3)cc2)cc1]

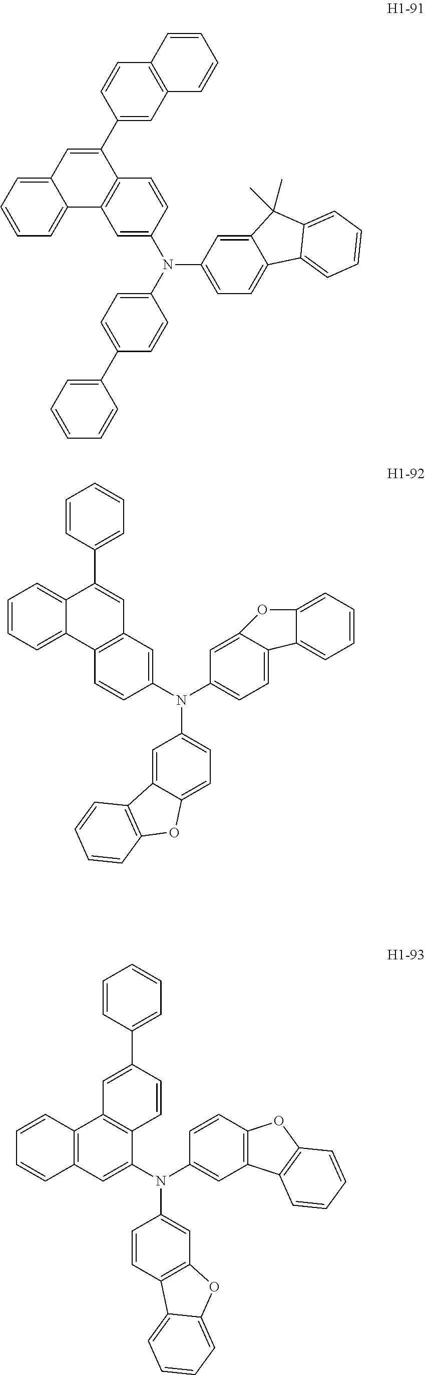

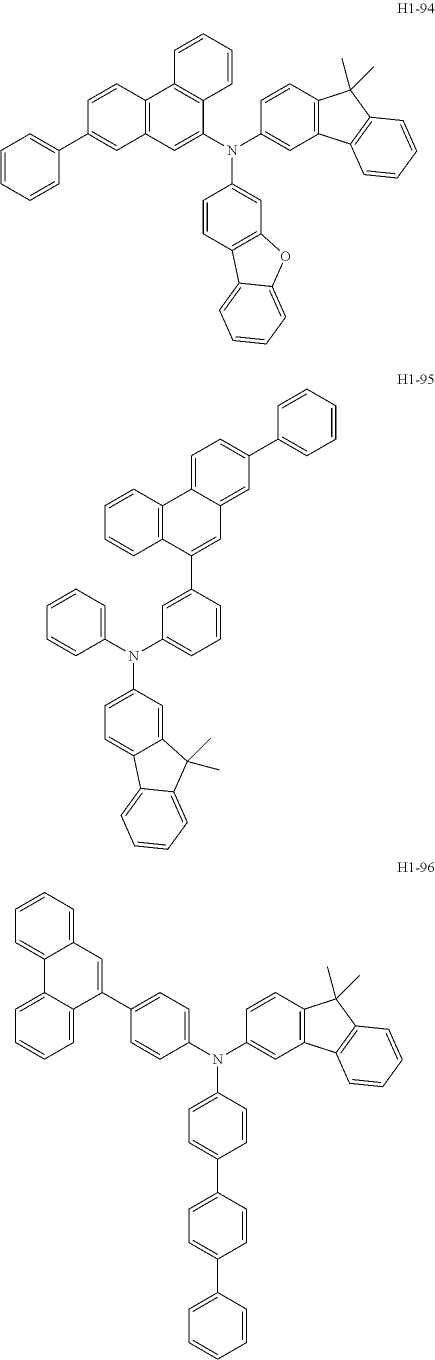 [SMILES: CC1(C)c2ccccc2-c2cc(N(c3ccc4c(c3)oc3ccccc34)c3cc4cc(-c5ccccc5)ccc4c4ccccc34)ccc21 ; CC1(C)c2ccccc2-c2ccc(N(c3ccccc3)c3cccc(-c4cc5cc(-c6ccccc6)ccc5c5ccccc45)c3)cc21 ; CC1(C)c2ccccc2-c2cc(N(c3ccc(-c4ccc(-c5ccccc5)cc4)cc3)c3ccc(-c4cc5ccccc5c5ccccc45)cc3)ccc21]

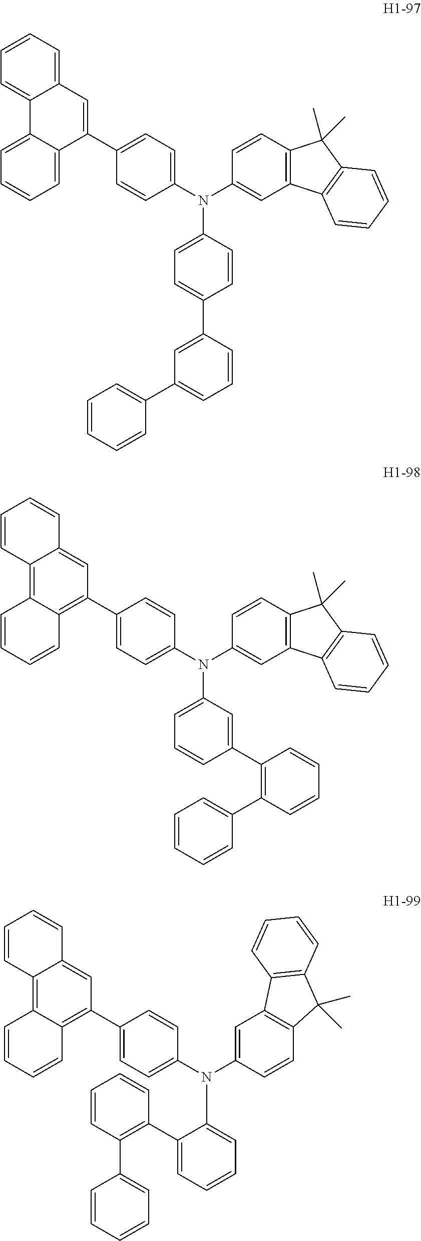 [SMILES: CC1(C)c2ccccc2-c2cc(N(c3ccc(-c4cccc(-c5ccccc5)c4)cc3)c3ccc(-c4cc5ccccc5c5ccccc45)cc3)ccc21 ; CC1(C)c2ccccc2-c2cc(N(c3ccc(-c4cc5ccccc5c5ccccc45)cc3)c3cccc(-c4ccccc4-c4ccccc4)c3)ccc21 ; CC1(C)c2ccccc2-c2cc(N(c3ccc(-c4cc5ccccc5c5ccccc45)cc3)c3ccccc3-c3ccccc3-c3ccccc3)ccc21]

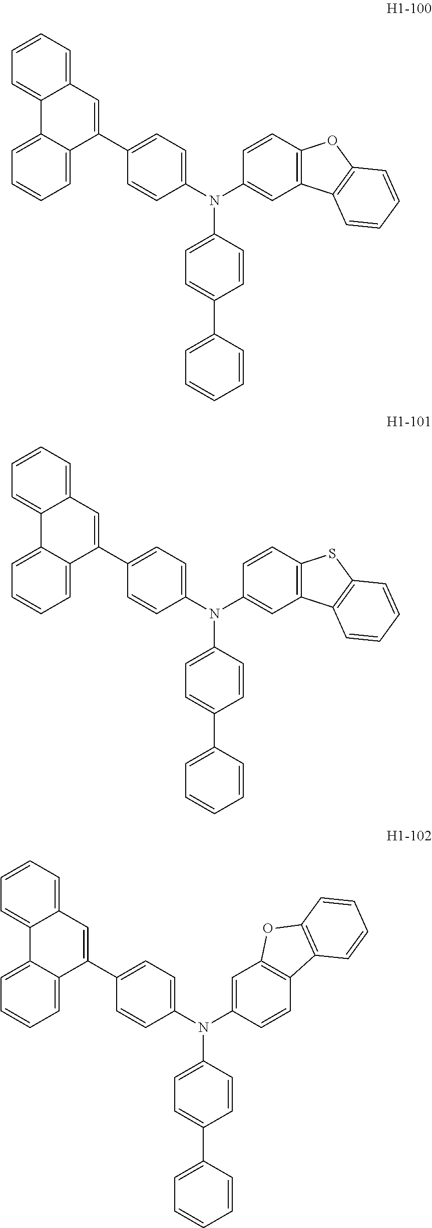 [SMILES: c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4sc5ccccc5c4c3)cc2)cc1 ; c1ccc(-c2ccc(N(c3ccc(-c4cc5ccccc5c5ccccc45)cc3)c3ccc4c(c3)oc3ccccc34)cc2)cc1]

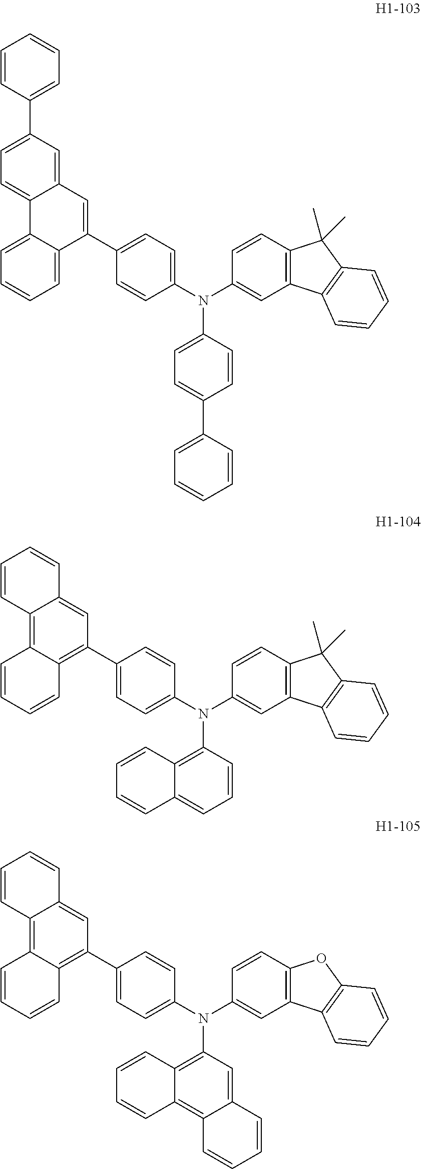 [SMILES: CC1(C)c2ccccc2-c2cc(N(c3ccc(-c4ccccc4)cc3)c3ccc(-c4cc5cc(-c6ccccc6)ccc5c5ccccc45)cc3)ccc21 ; CC1(C)c2ccccc2-c2cc(N(c3ccc(-c4cc5ccccc5c5ccccc45)cc3)c3cccc4ccccc34)ccc21 ; c1ccc2c(c1)cc(-c1ccc(N(c3ccc4oc5ccccc5c4c3)c3cc4ccccc4c4ccccc34)cc1)c1ccccc12]

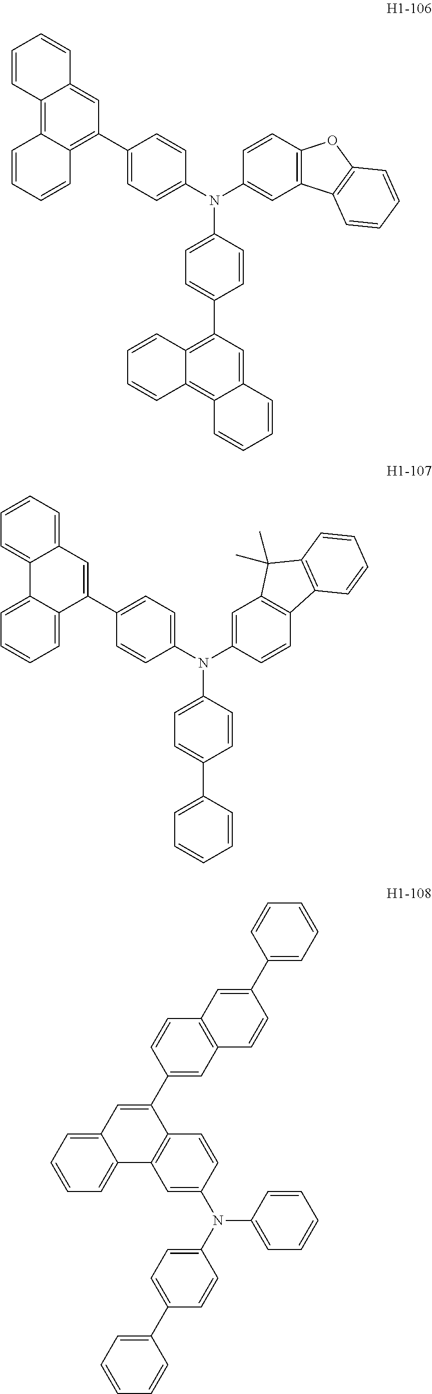 [SMILES: c1ccc2c(c1)cc(-c1ccc(N(c3ccc(-c4cc5ccccc5c5ccccc45)cc3)c3ccc4oc5ccccc5c4c3)cc1)c1ccccc12 ; CC1(C)c2ccccc2-c2ccc(N(c3ccc(-c4ccccc4)cc3)c3ccc(-c4cc5ccccc5c5ccccc45)cc3)cc21 ; c1ccc(-c2ccc(N(c3ccccc3)c3ccc4c(-c5ccc6cc(-c7ccccc7)ccc6c5)cc5ccccc5c4c3)cc2)cc1]

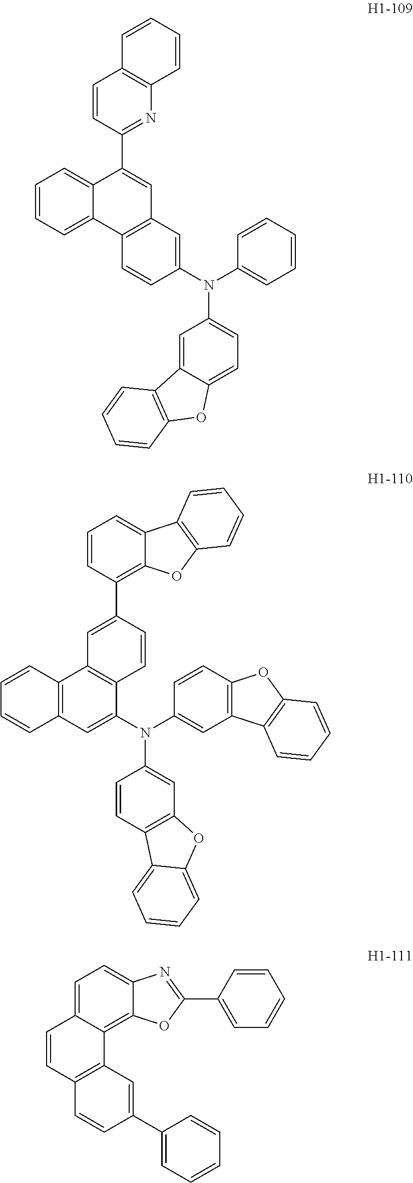 [SMILES: c1ccc(N(c2ccc3c(c2)cc(-c2ccc4ccccc4n2)c2ccccc23)c2ccc3oc4ccccc4c3c2)cc1 ; c1ccc2c(c1)cc(N(c1ccc3c(c1)oc1ccccc13)c1ccc3oc4ccccc4c3c1)c1ccc(-c3cccc4c3oc3ccccc34)cc12 ; c1ccc(-c2ccc3ccc4ccc5nc(-c6ccccc6)oc5c4c3c2)cc1]

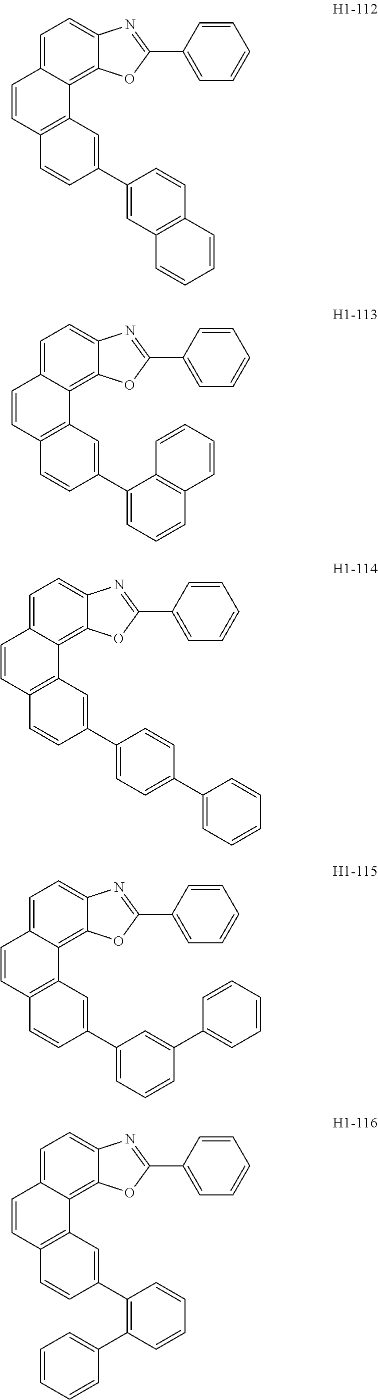

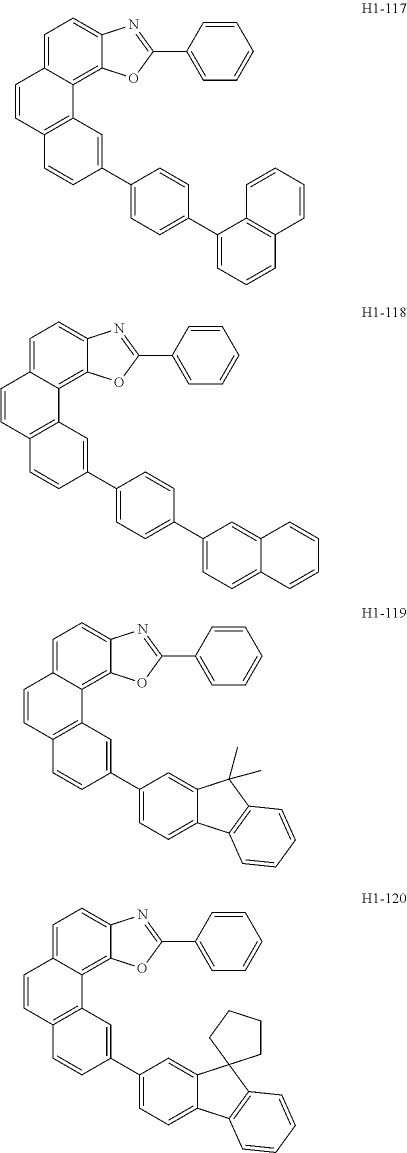

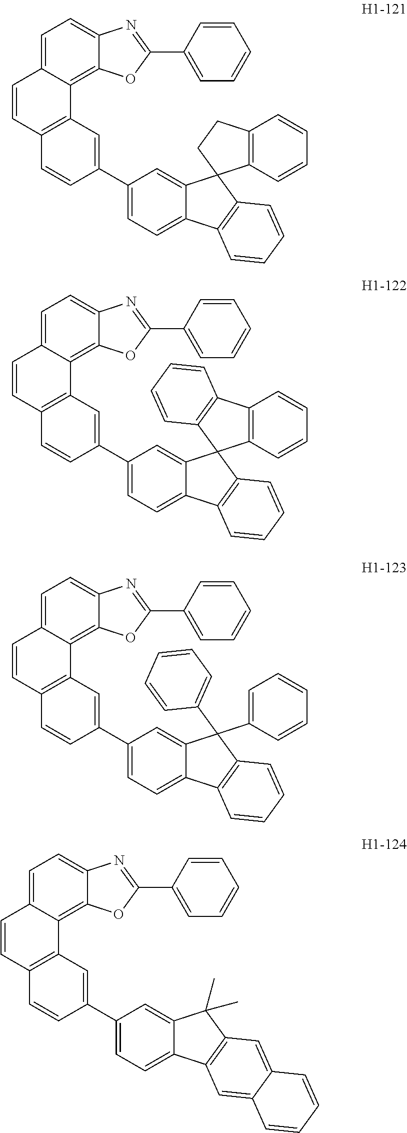

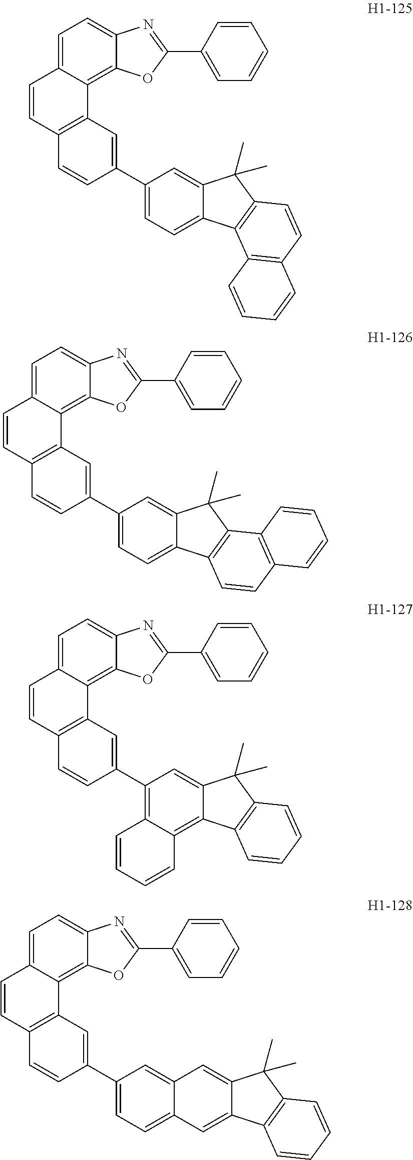

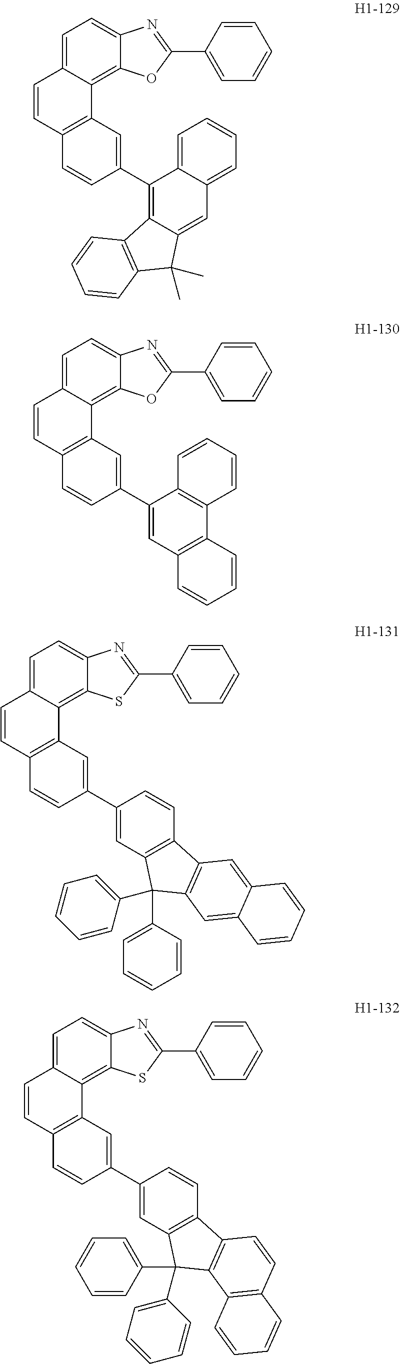

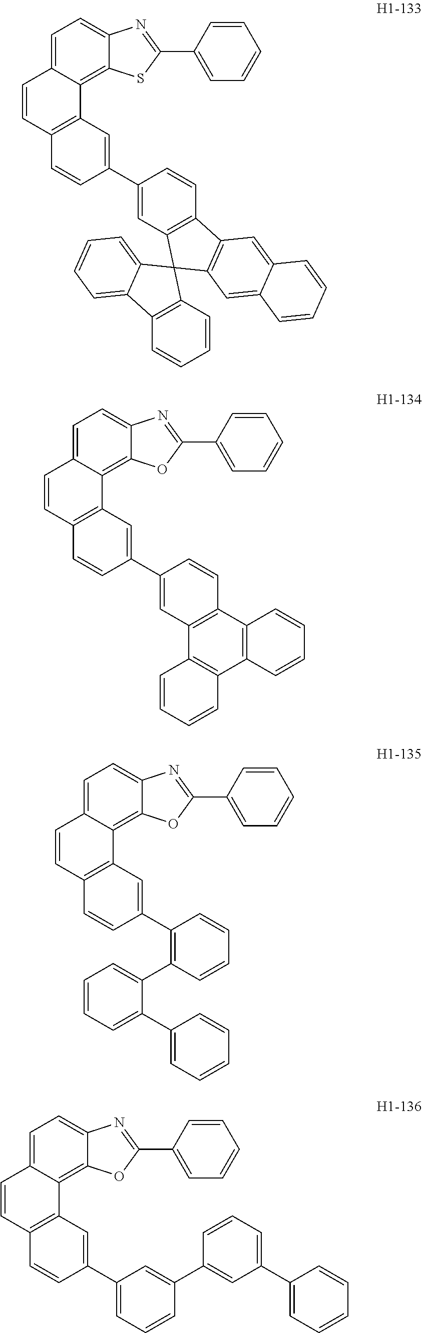

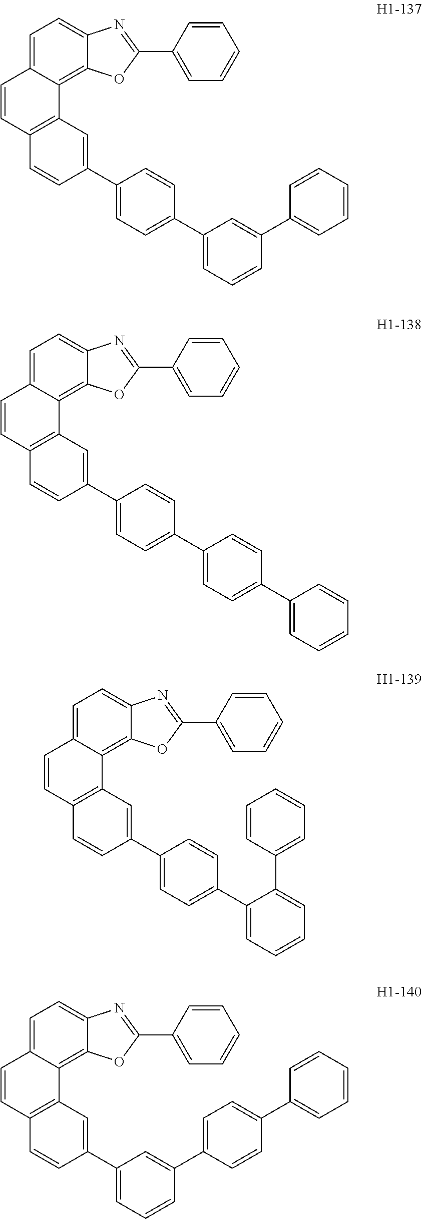 [SMILES: c1ccc(-c2cccc(-c3ccc(-c4ccc5ccc6ccc7nc(-c8ccccc8)oc7c6c5c4)cc3)c2)cc1 ; c1ccc(-c2ccc(-c3ccc(-c4ccc5ccc6ccc7nc(-c8ccccc8)oc7c6c5c4)cc3)cc2)cc1 ; c1ccc(-c2nc3ccc4ccc5ccc(-c6ccc(-c7ccccc7-c7ccccc7)cc6)cc5c4c3o2)cc1 ; c1ccc(-c2ccc(-c3cccc(-c4ccc5ccc6ccc7nc(-c8ccccc8)oc7c6c5c4)c3)cc2)cc1]

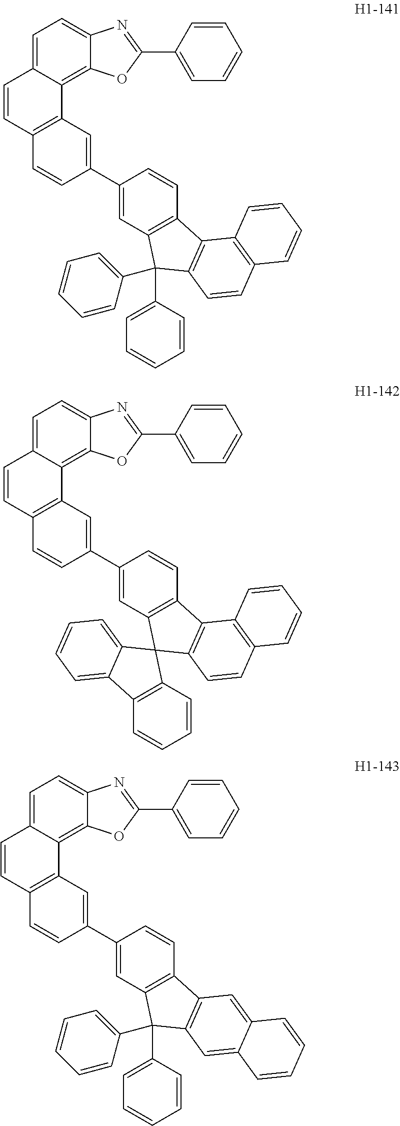

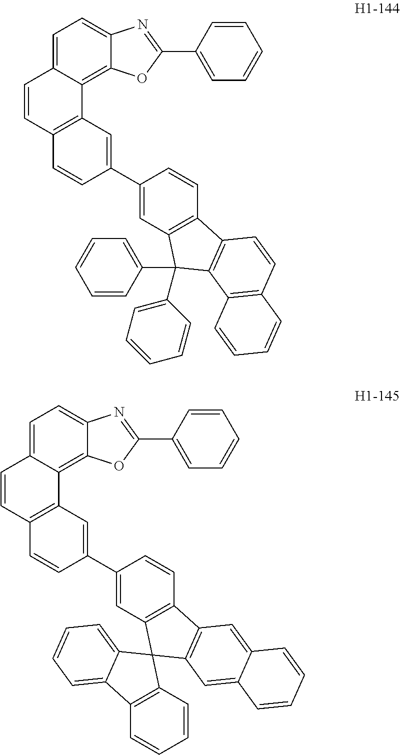 [SMILES: c1ccc(-c2nc3ccc4ccc5ccc(-c6ccc7c(c6)C(c6ccccc6)(c6ccccc6)c6c-7ccc7ccccc67)cc5c4c3o2)cc1 ; c1ccc(-c2nc3ccc4ccc5ccc(-c6ccc7c(c6)C6(c8ccccc8-c8ccccc86)c6cc8ccccc8cc6-7)cc5c4c3o2)cc1]

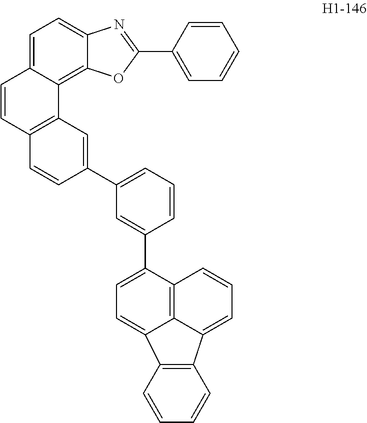 [SMILES: c1ccc(-c2nc3ccc4ccc5ccc(-c6cccc(-c7ccc8c9c(cccc79)-c7ccccc7-8)c6)cc5c4c3o2)cc1]

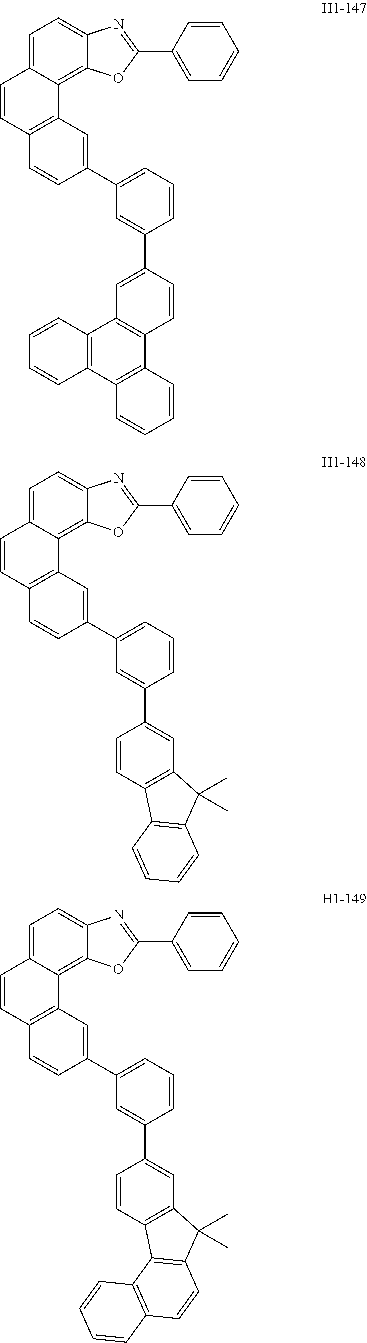

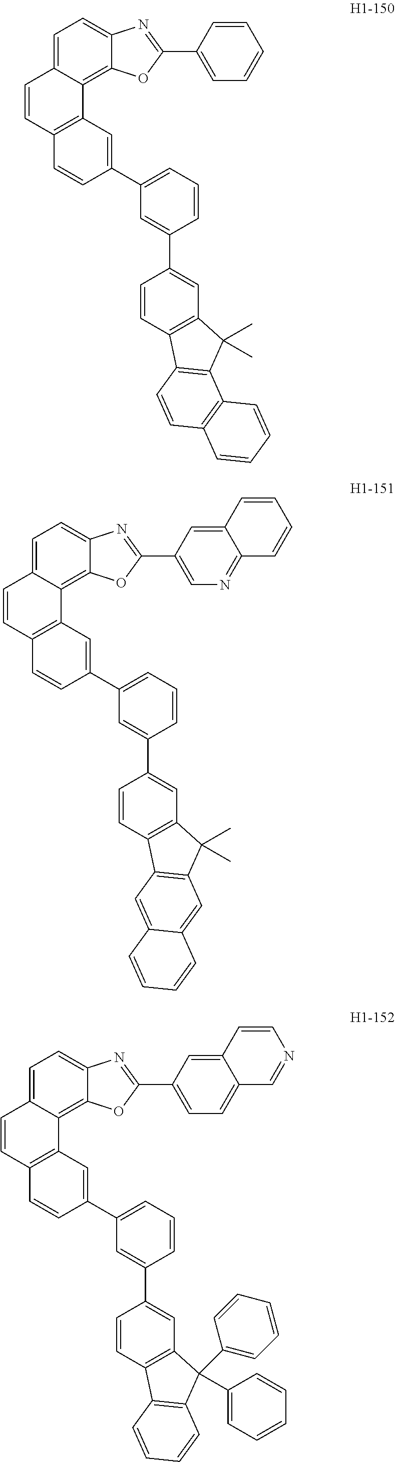

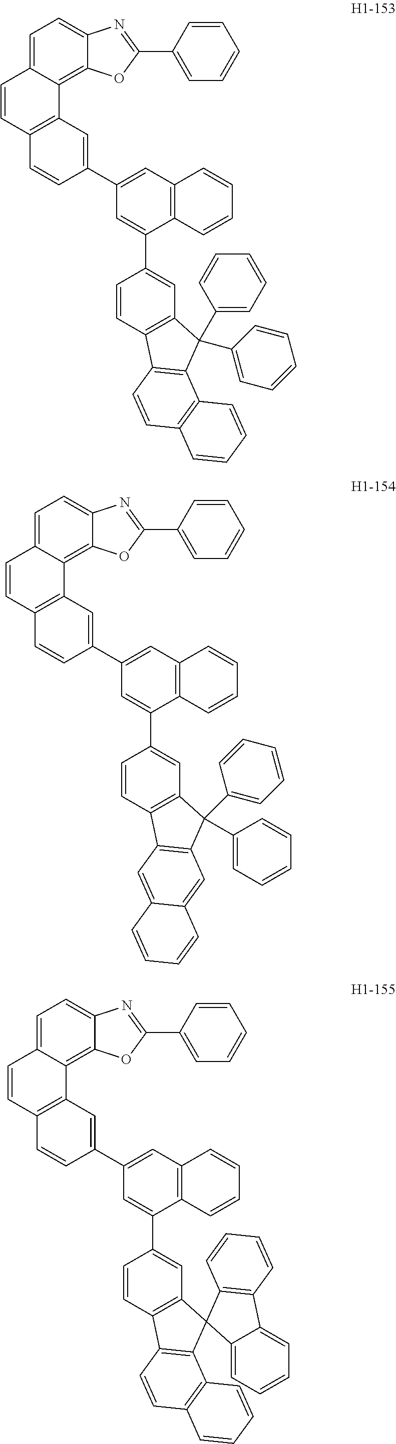

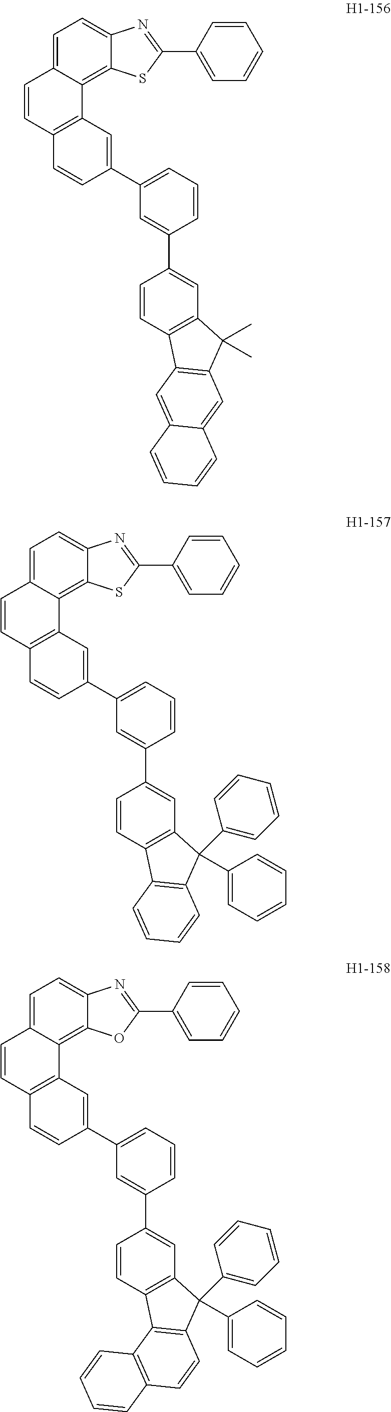

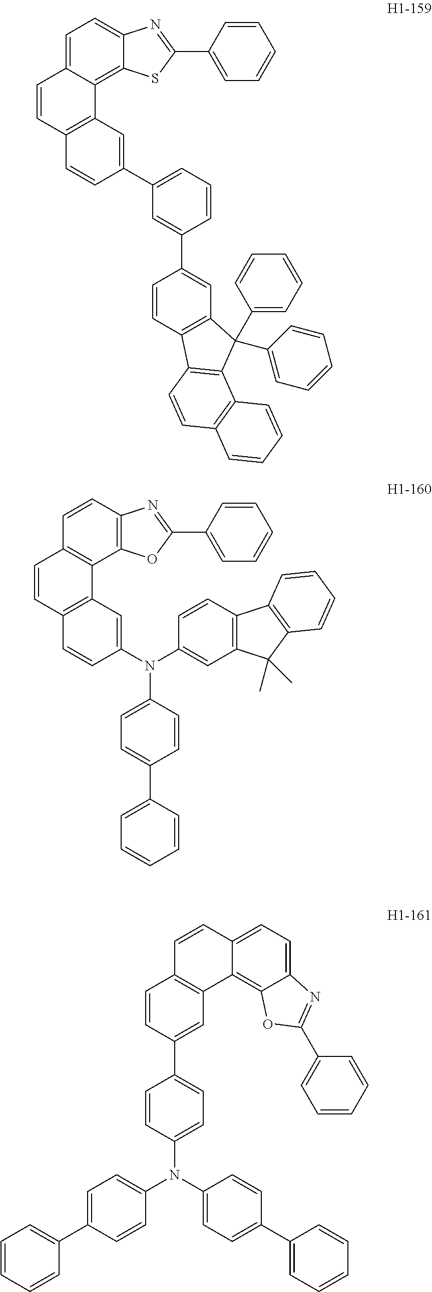

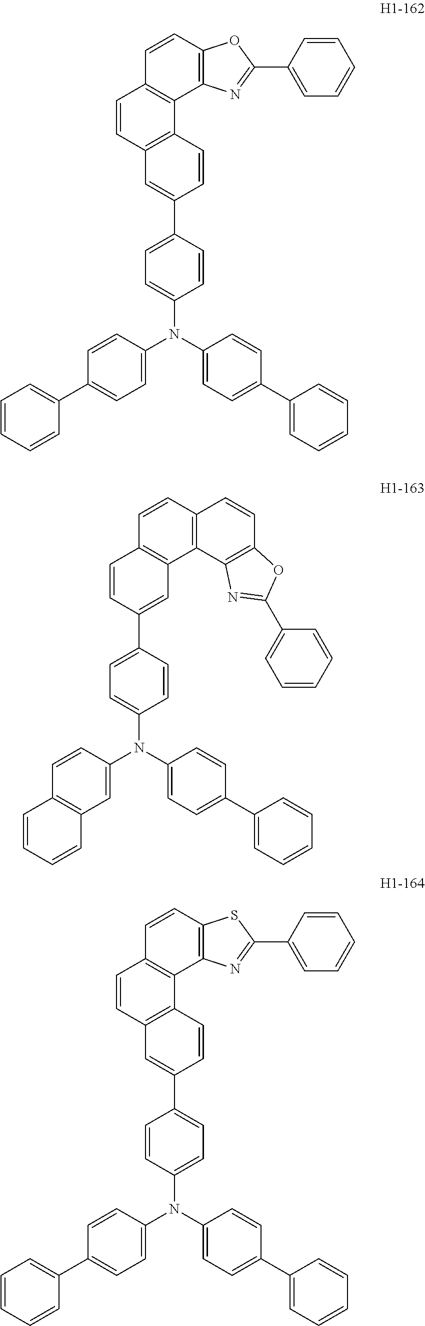 [SMILES: c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1]

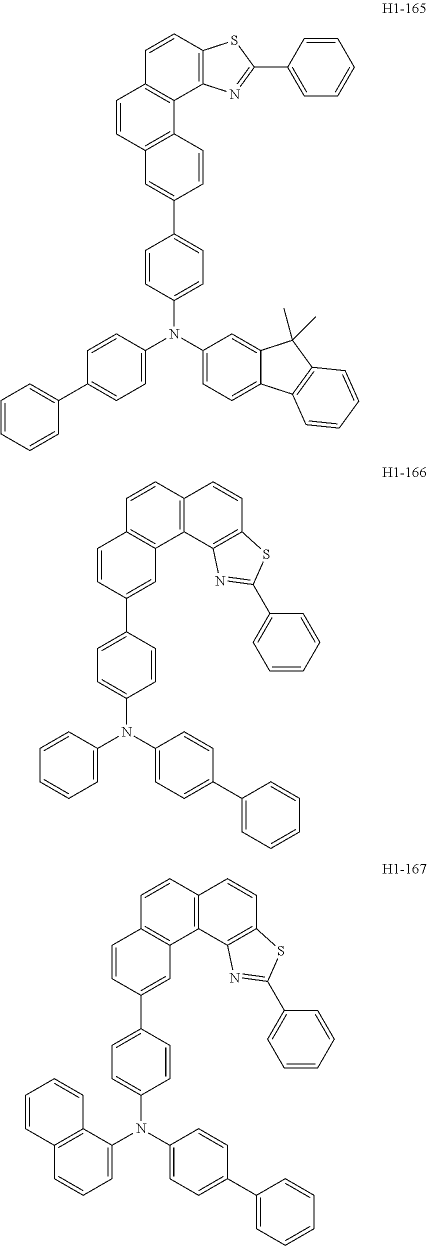 [SMILES: CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1]

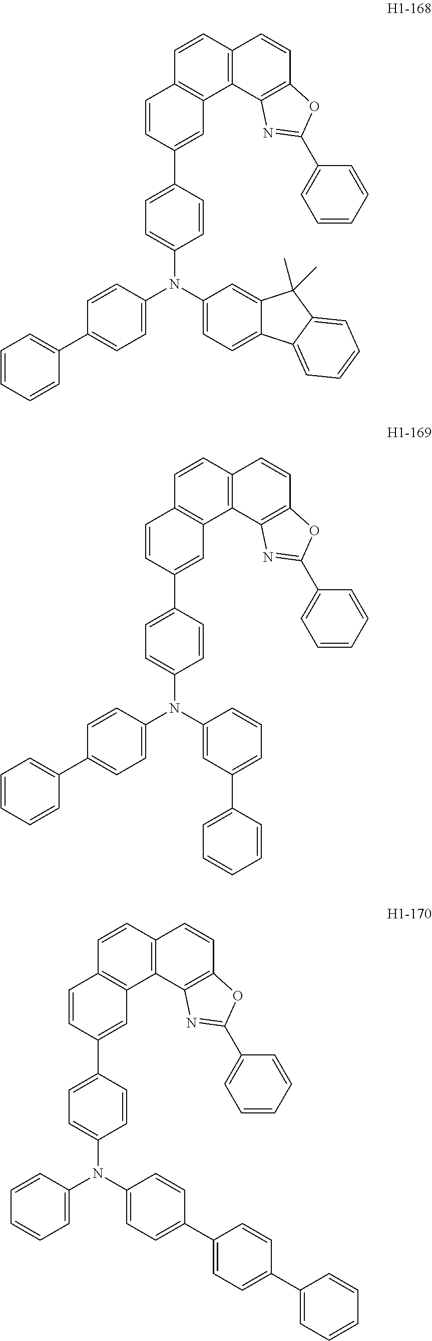 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

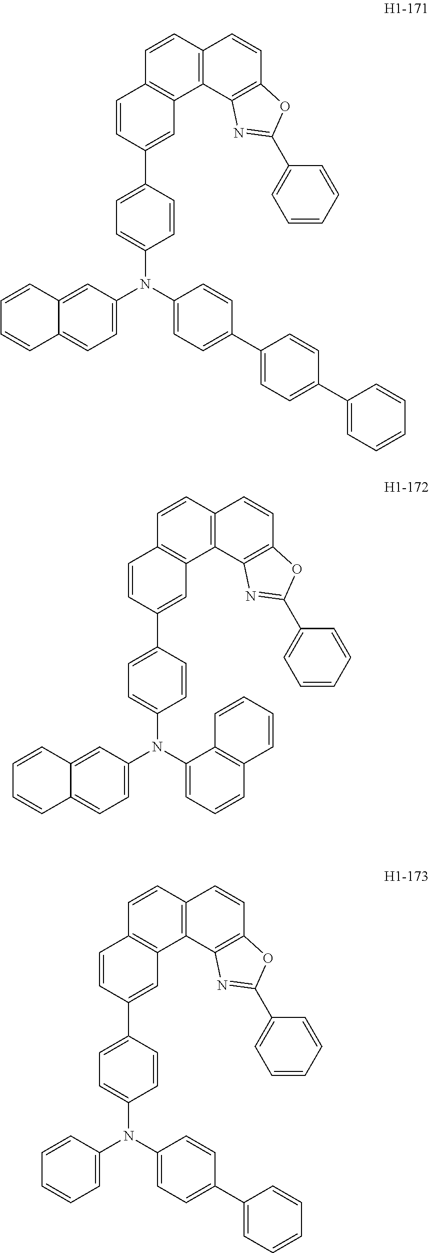 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1]

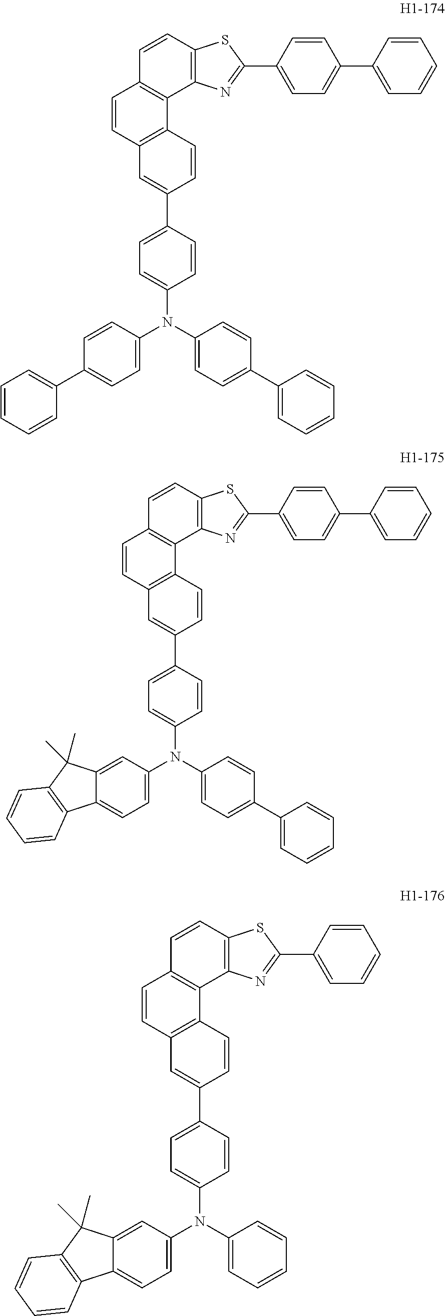 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21]

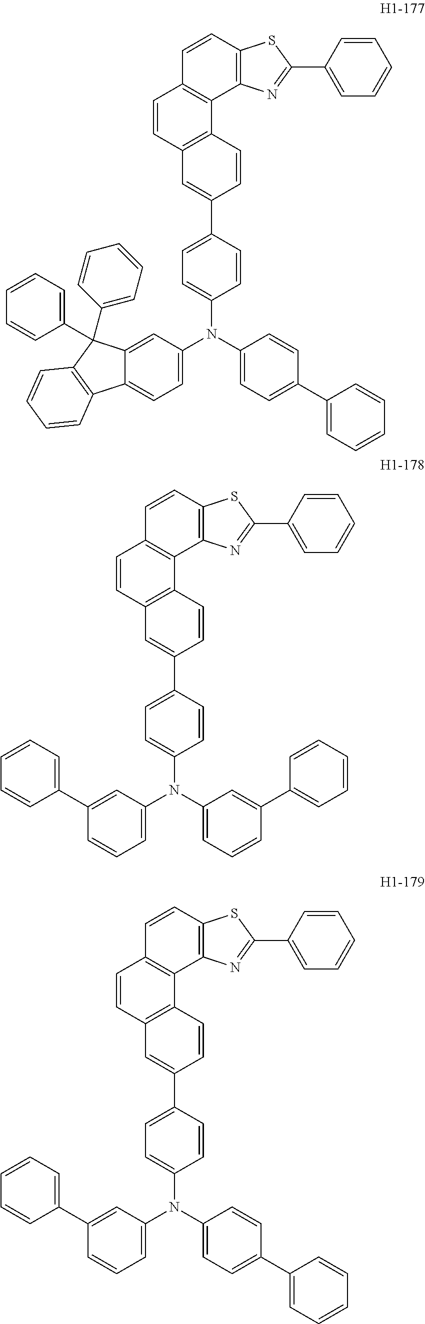 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

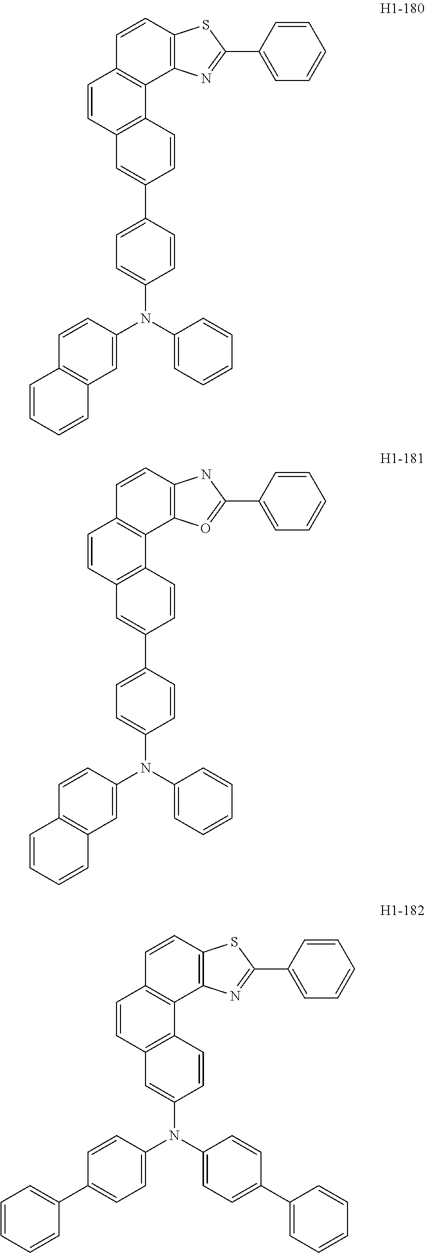

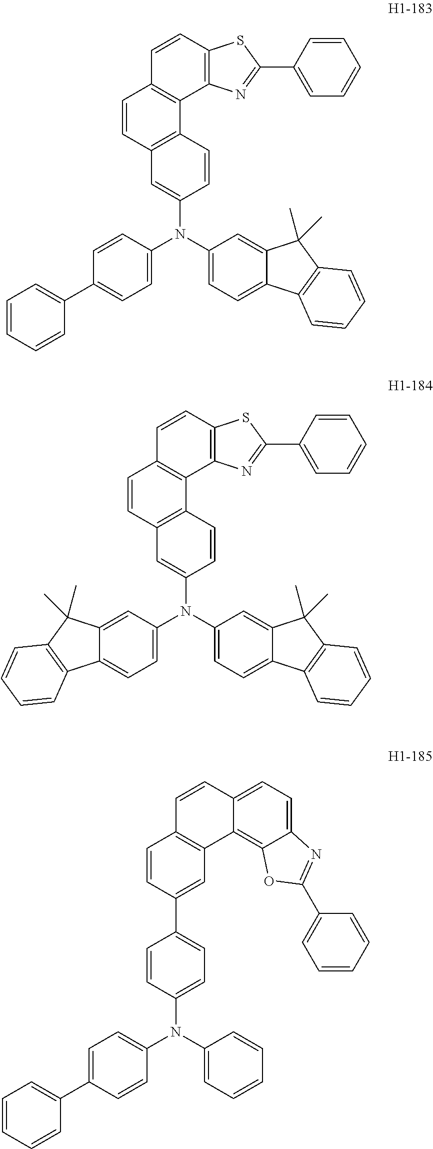 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

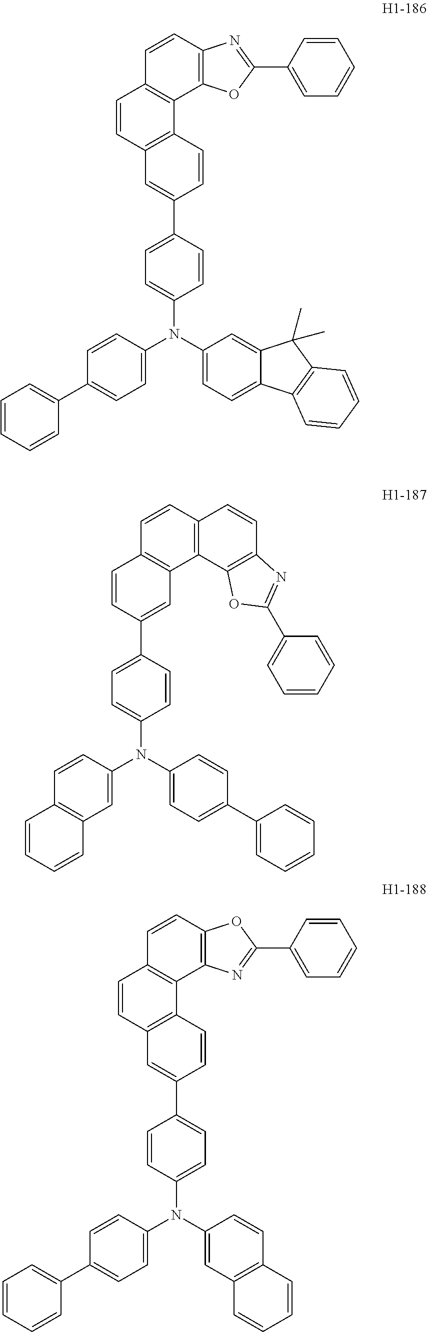 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

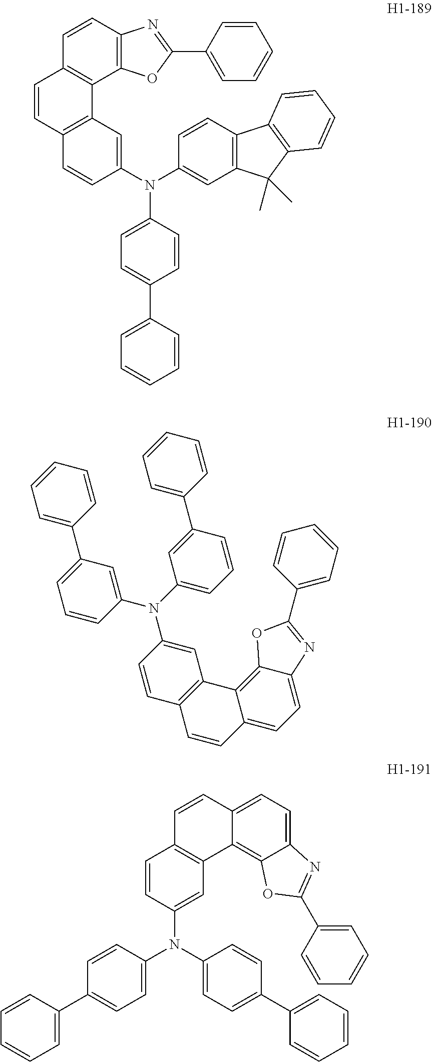 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2cccc(N(c3cccc(-c4ccccc4)c3)c3ccc4ccc5ccc6nc(-c7ccccc7)oc6c5c4c3)c2)cc1 ; c1ccc(-c2ccc(N(c3ccc(-c4ccccc4)cc3)c3ccc4ccc5ccc6nc(-c7ccccc7)oc6c5c4c3)cc2)cc1]

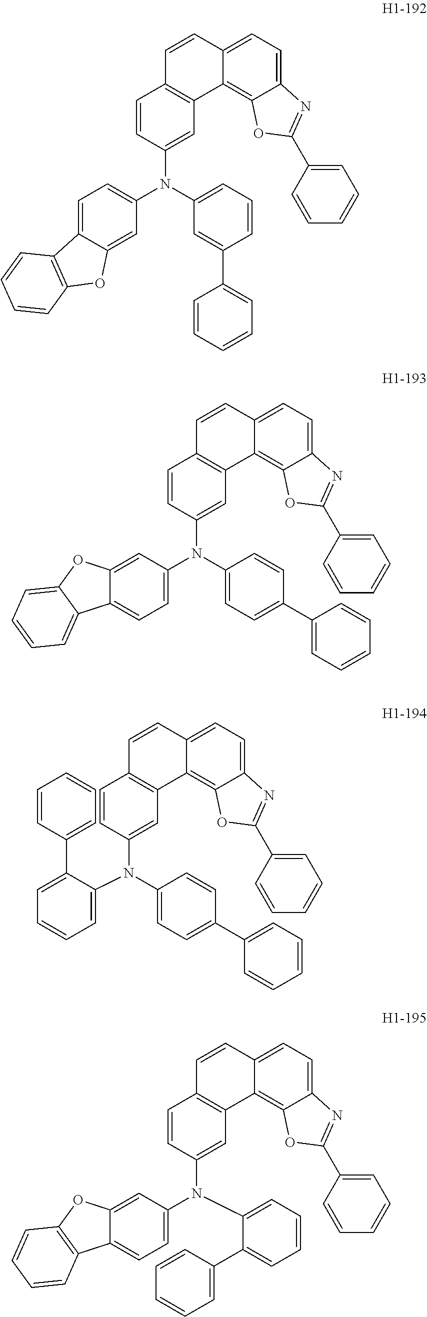 [SMILES: c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3ccccc3-c3ccccc3)cc2)cc1 ; c1ccc(-c2nc3ccc4ccc5ccc(N(c6ccc7c(c6)oc6ccccc67)c6ccccc6-c6ccccc6)cc5c4c3o2)cc1]

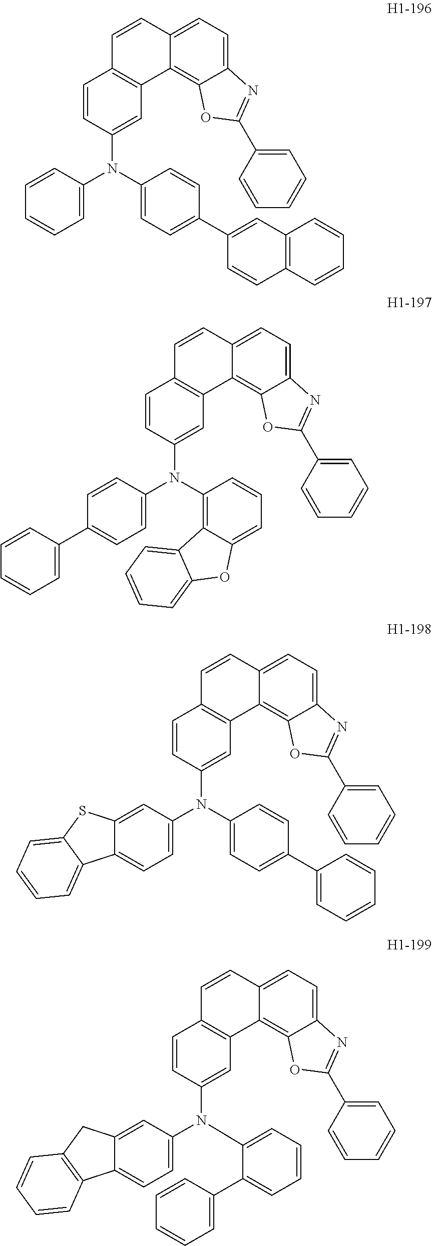

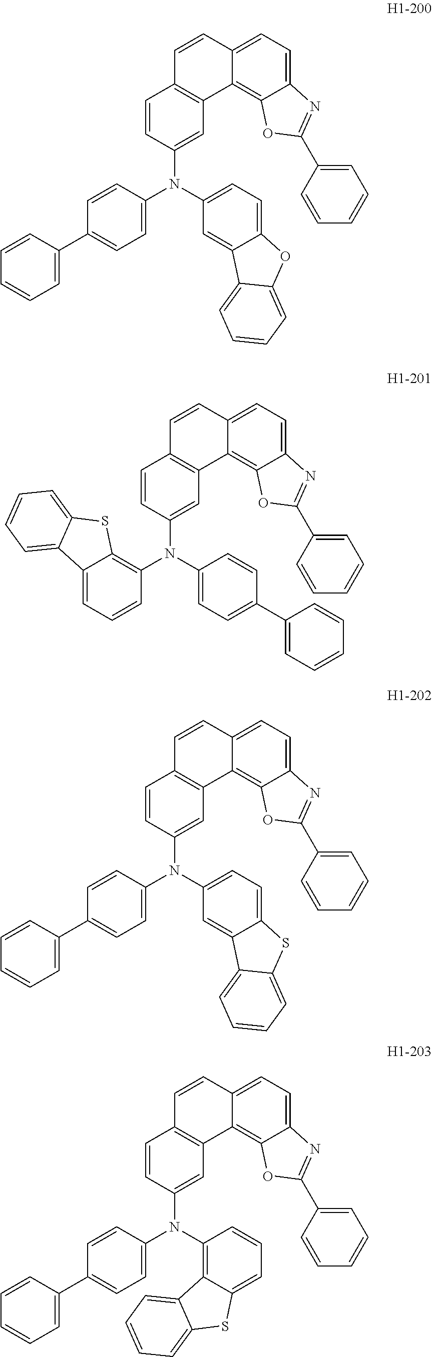

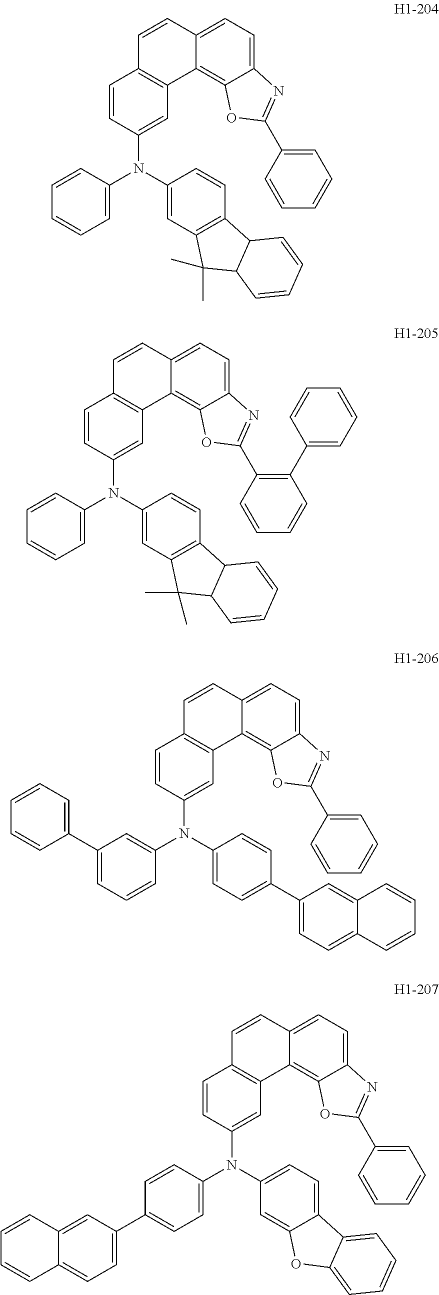

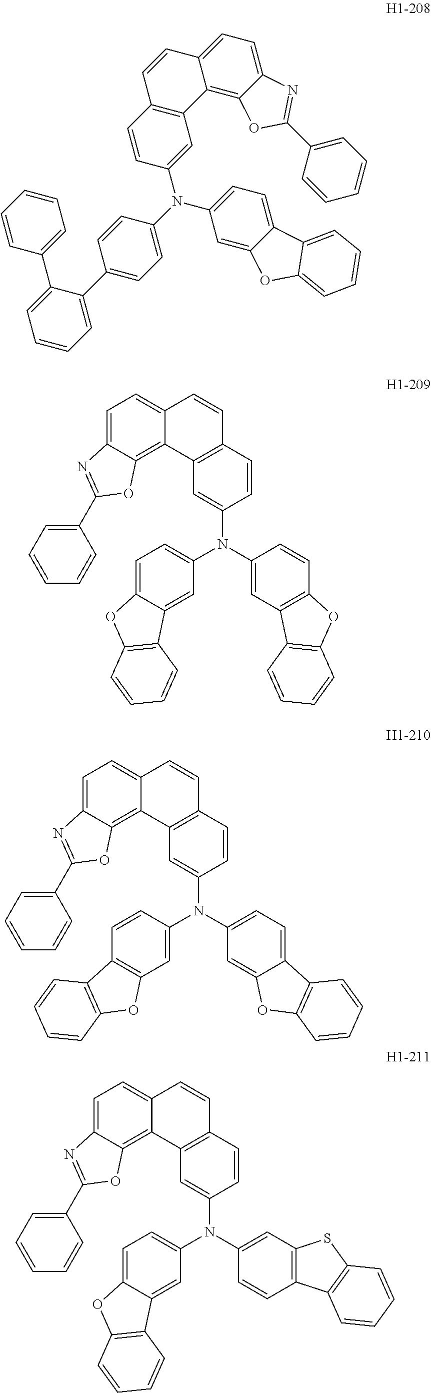 [SMILES: c1ccc(-c2nc3ccc4ccc5ccc(N(c6ccc(-c7ccccc7-c7ccccc7)cc6)c6ccc7c(c6)oc6ccccc67)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1]

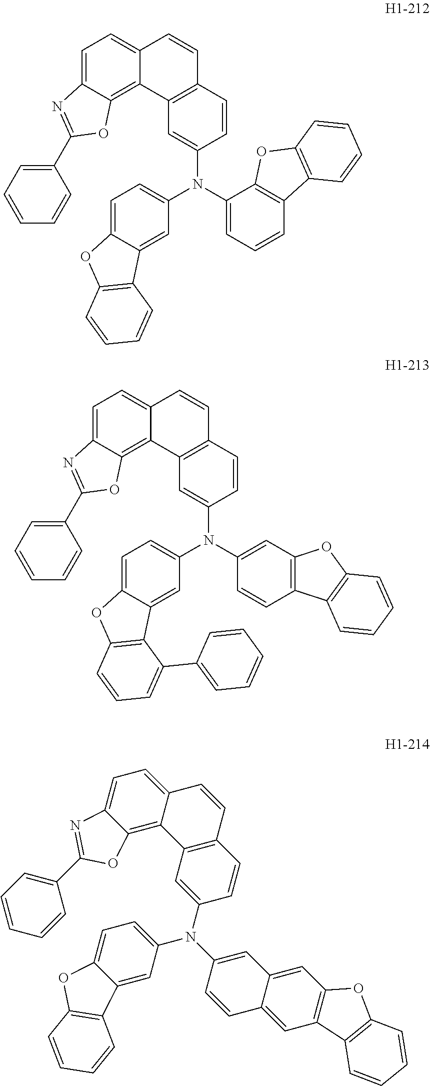 [SMILES: c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1]

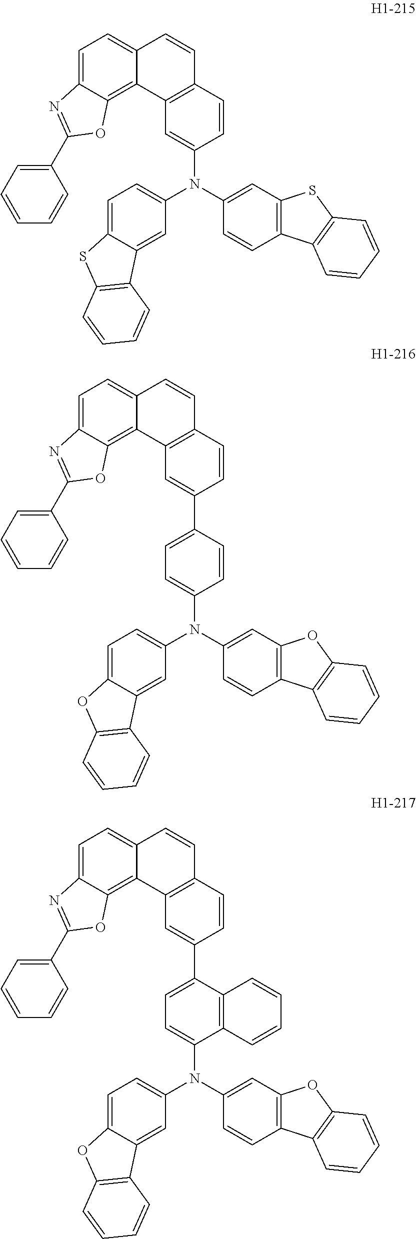

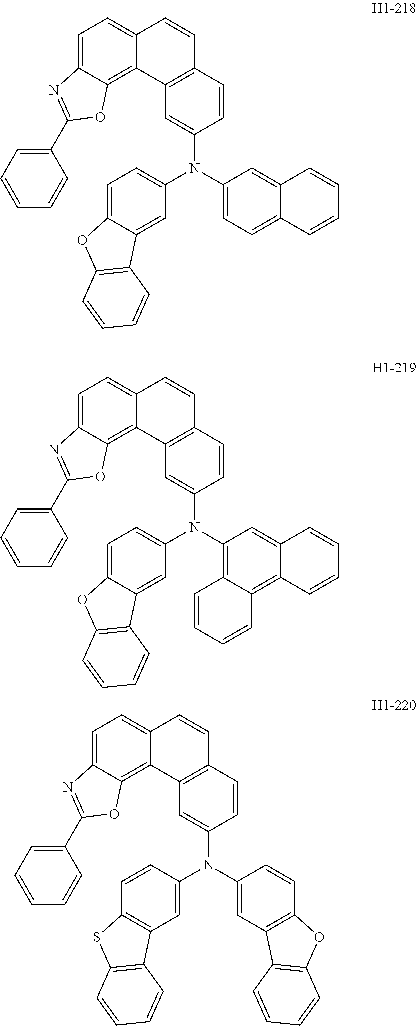

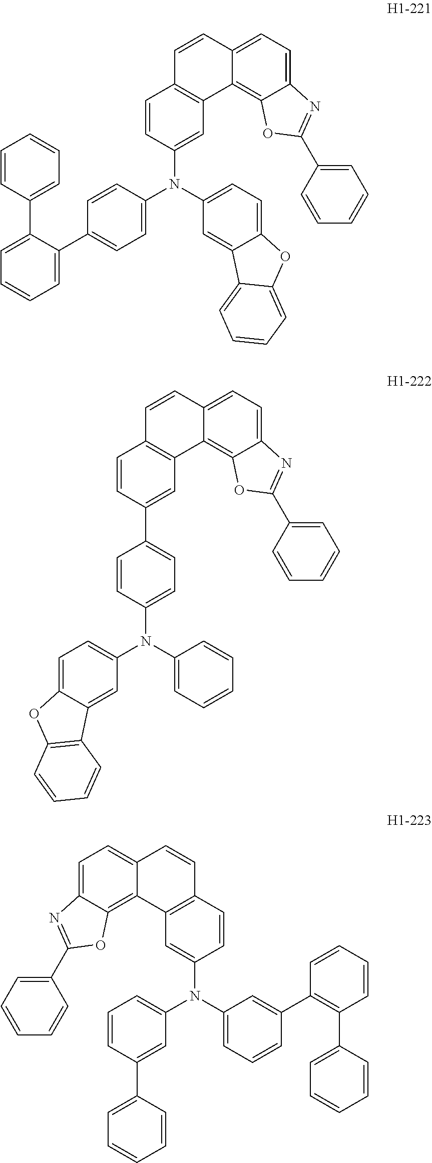 [SMILES: c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1]

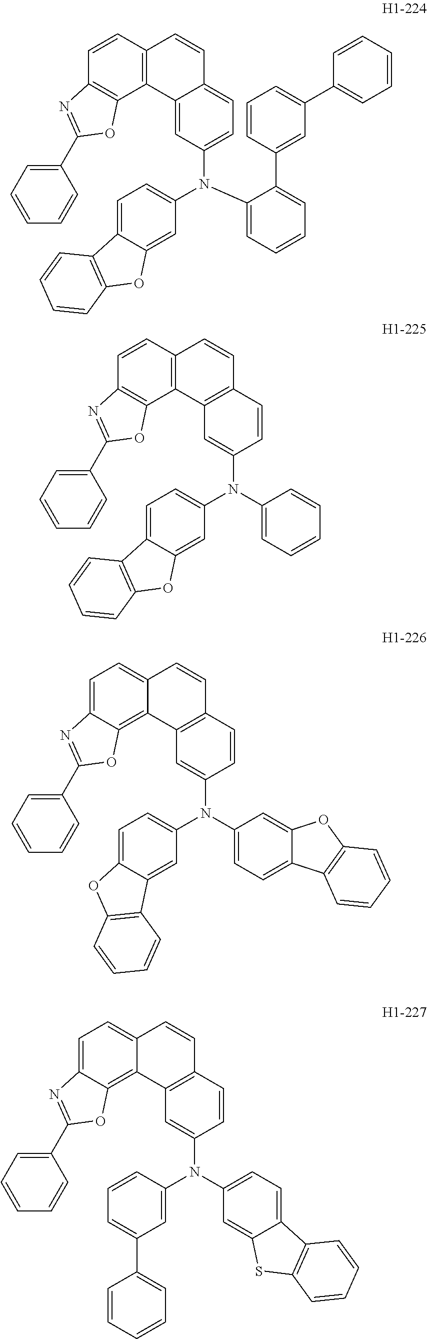

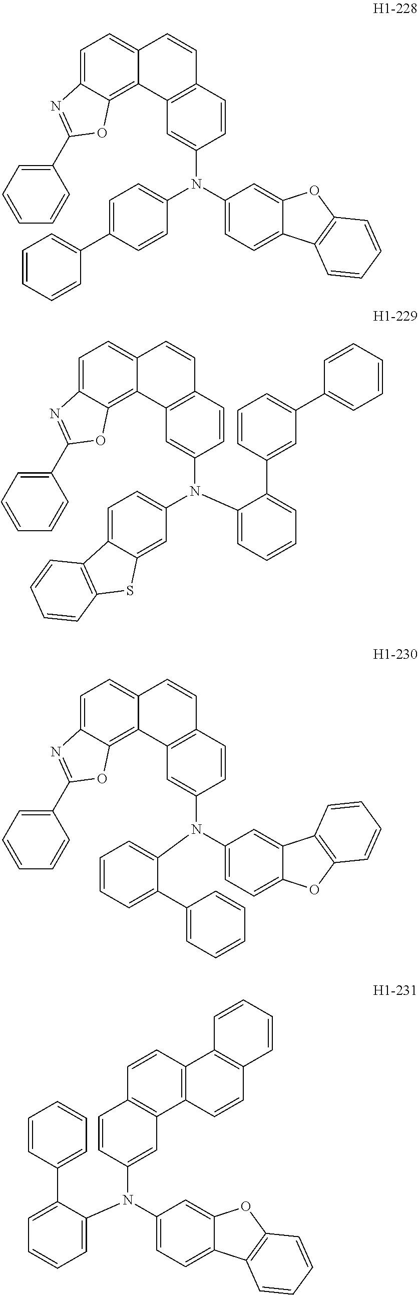 [SMILES: c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2cccc(-c3ccccc3N(c3ccc4c(c3)sc3ccccc34)c3ccc4ccc5ccc6nc(-c7ccccc7)oc6c5c4c3)c2)cc1 ; c1ccc(-c2nc3ccc4ccc5ccc(N(c6ccc7oc8ccccc8c7c6)c6ccccc6-c6ccccc6)cc5c4c3o2)cc1 ; c1ccc(-c2ccccc2N(c2ccc3c(c2)oc2ccccc23)c2ccc3ccc4c5ccccc5ccc4c3c2)cc1]

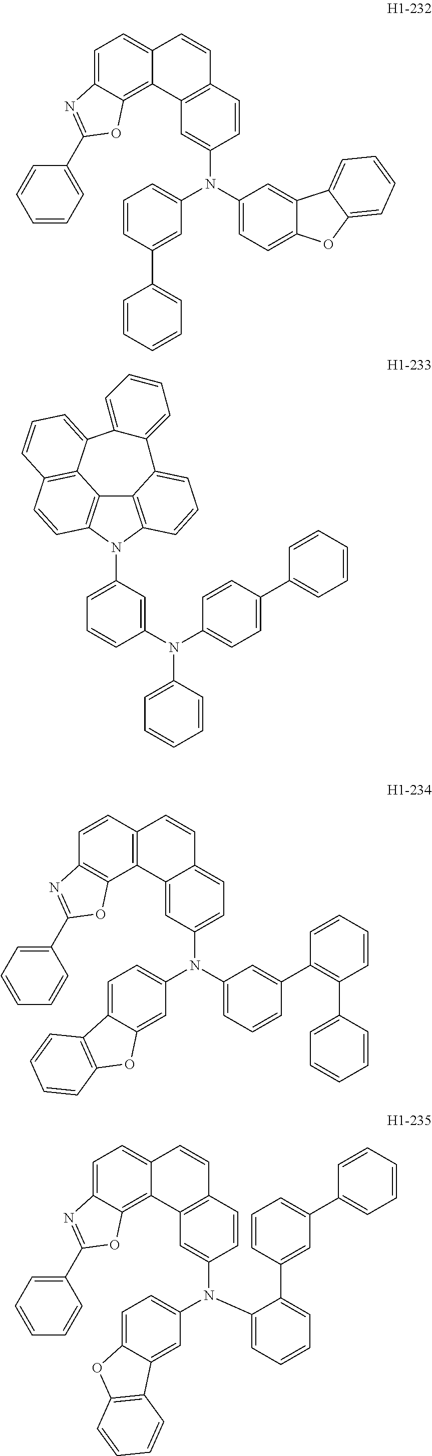

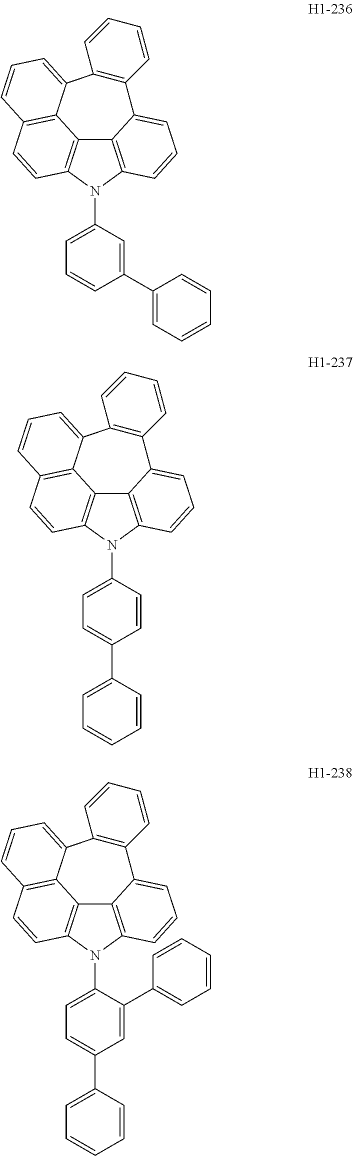

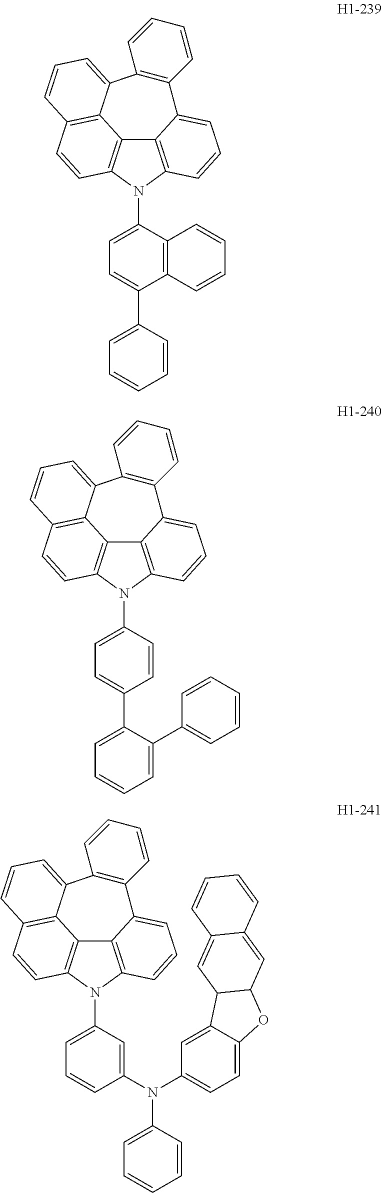

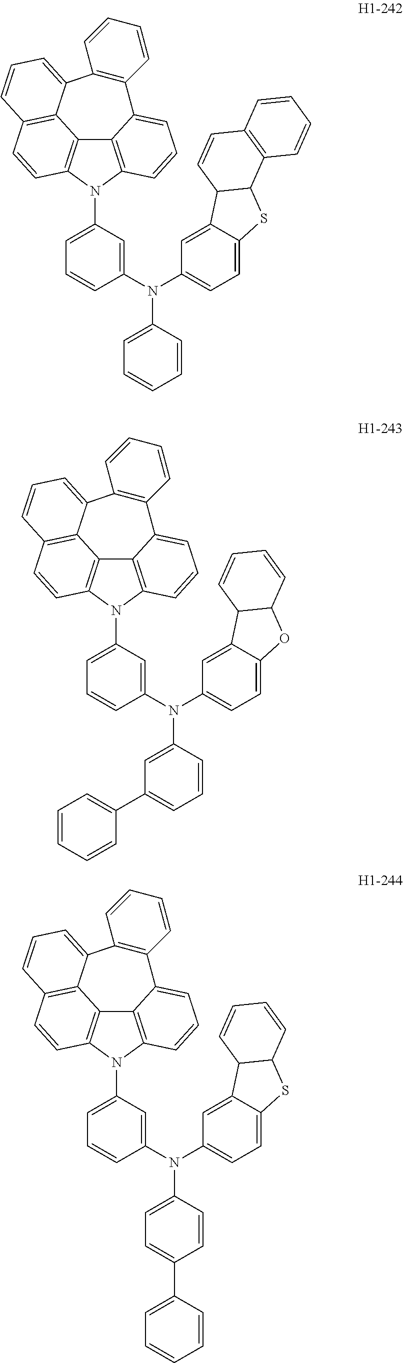 [SMILES: C1=CC2c3cc(N(c4ccccc4)c4cccc(-n5c6cccc7c6c6c8c(cccc8ccc65)-c5ccccc5-7)c4)ccc3SC2c2ccccc21 ; C1=CC2Oc3ccc(N(c4cccc(-c5ccccc5)c4)c4cccc(-n5c6cccc7c6c6c8c(cccc8ccc65)-c5ccccc5-7)c4)cc3C2C=C1 ; C1=CC2Sc3ccc(N(c4ccc(-c5ccccc5)cc4)c4cccc(-n5c6cccc7c6c6c8c(cccc8ccc65)-c5ccccc5-7)c4)cc3C2C=C1]

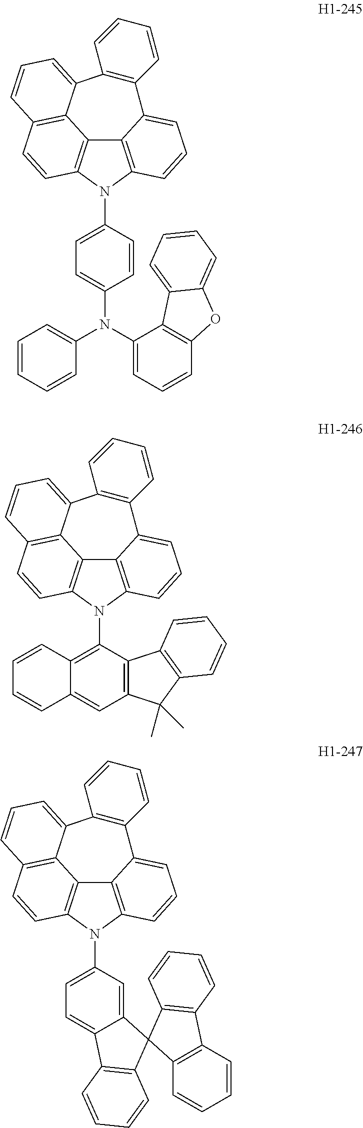

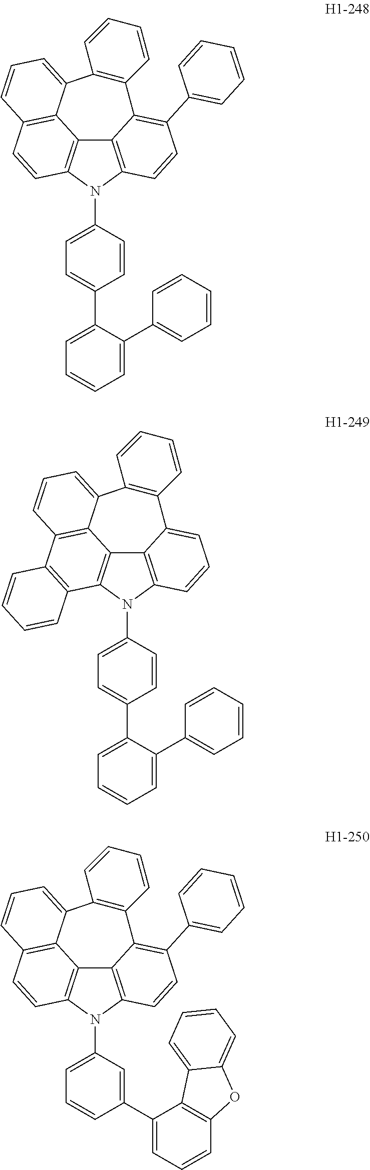

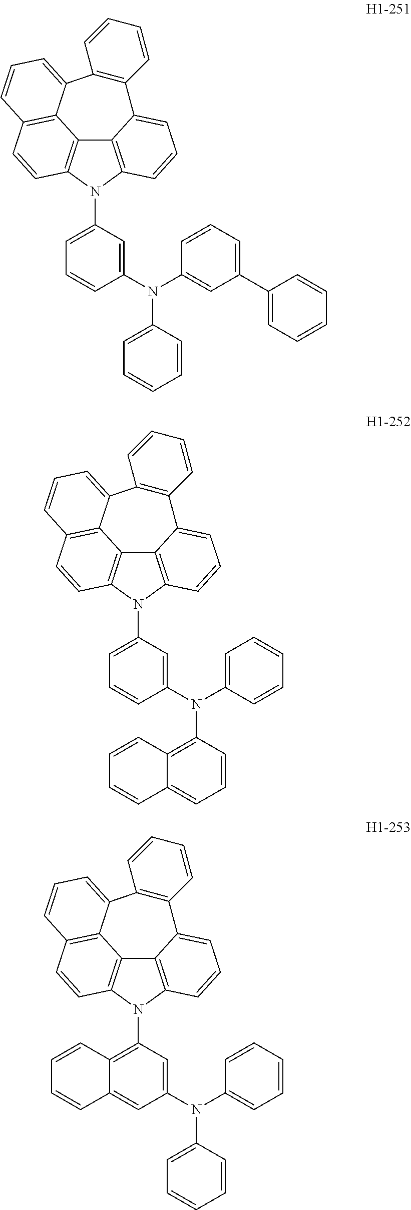

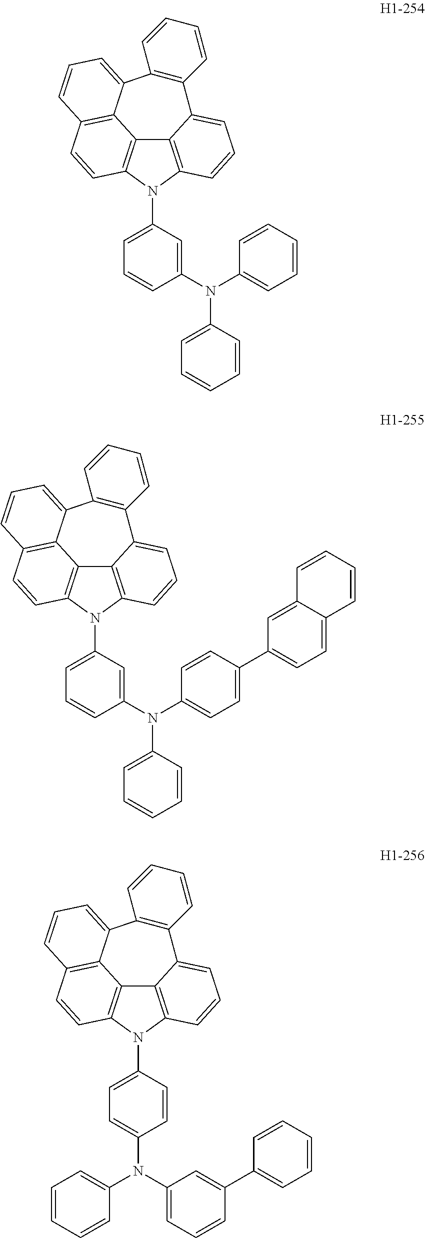 [SMILES: c1ccc(N(c2ccccc2)c2cccc(-n3c4cccc5c4c4c6c(cccc6ccc43)-c3ccccc3-5)c2)cc1 ; c1ccc(N(c2ccc(-c3ccc4ccccc4c3)cc2)c2cccc(-n3c4cccc5c4c4c6c(cccc6ccc43)-c3ccccc3-5)c2)cc1 ; c1ccc(-c2cccc(N(c3ccccc3)c3ccc(-n4c5cccc6c5c5c7c(cccc7ccc54)-c4ccccc4-6)cc3)c2)cc1]

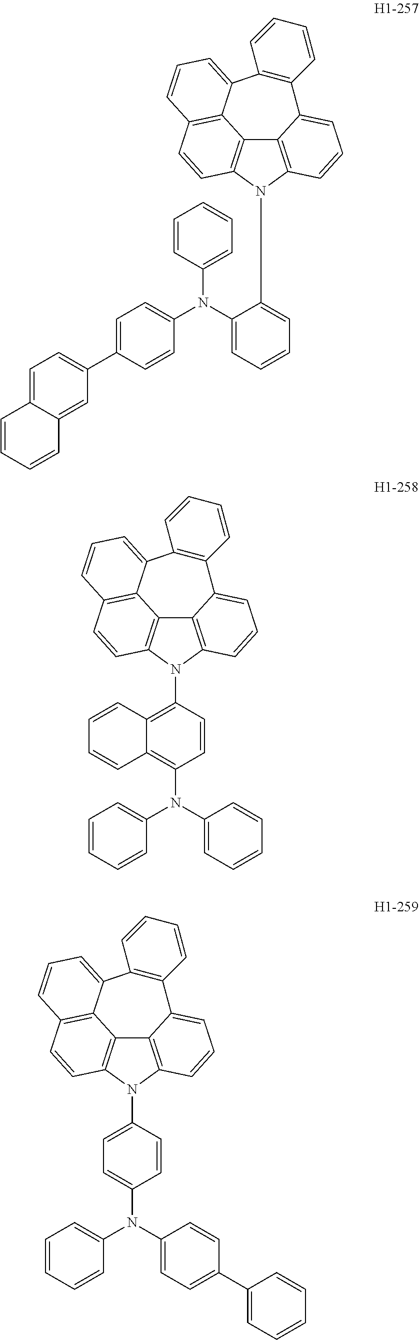 [SMILES: c1ccc(N(c2ccc(-c3ccc4ccccc4c3)cc2)c2ccccc2-n2c3cccc4c3c3c5c(cccc5ccc32)-c2ccccc2-4)cc1 ; c1ccc(N(c2ccccc2)c2ccc(-n3c4cccc5c4c4c6c(cccc6ccc43)-c3ccccc3-5)c3ccccc23)cc1 ; c1ccc(-c2ccc(N(c3ccccc3)c3ccc(-n4c5cccc6c5c5c7c(cccc7ccc54)-c4ccccc4-6)cc3)cc2)cc1]

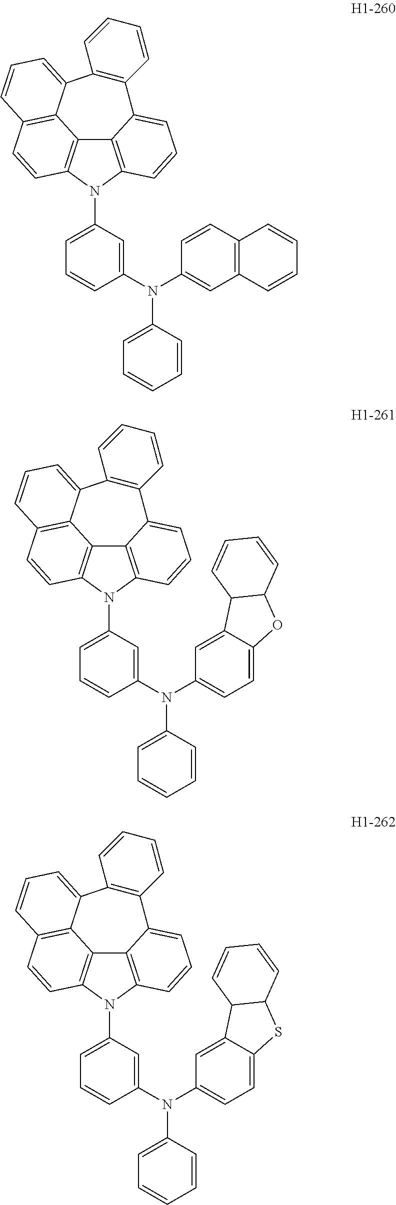

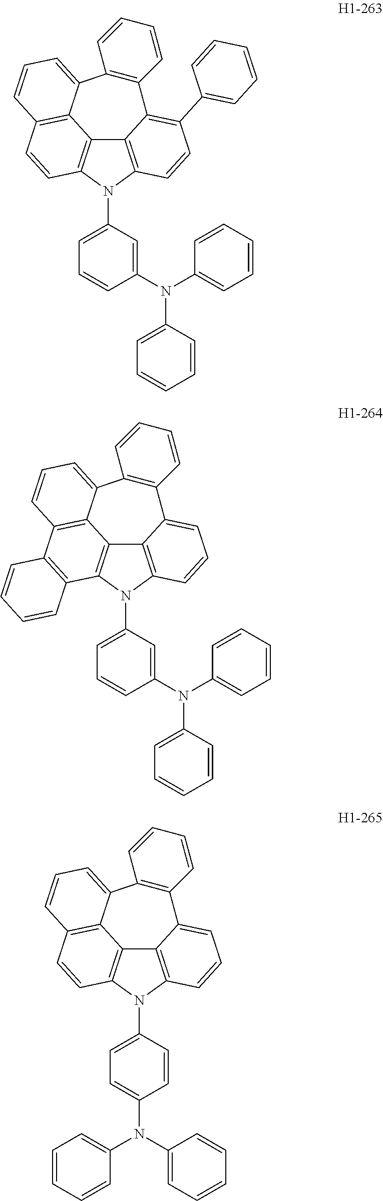

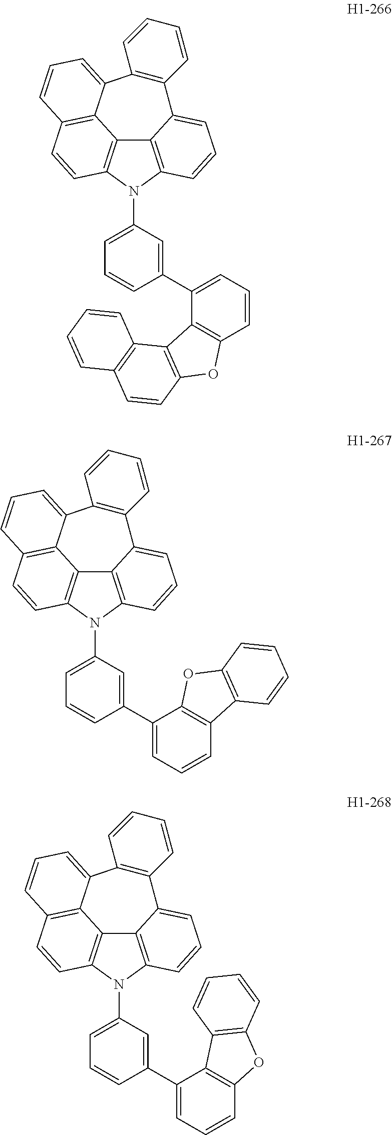 [SMILES: c1cc(-c2cccc3oc4ccc5ccccc5c4c23)cc(-n2c3cccc4c3c3c5c(cccc5ccc32)-c2ccccc2-4)c1 ; c1cc(-c2cccc3c2oc2ccccc23)cc(-n2c3cccc4c3c3c5c(cccc5ccc32)-c2ccccc2-4)c1 ; c1cc(-c2cccc3oc4ccccc4c23)cc(-n2c3cccc4c3c3c5c(cccc5ccc32)-c2ccccc2-4)c1]

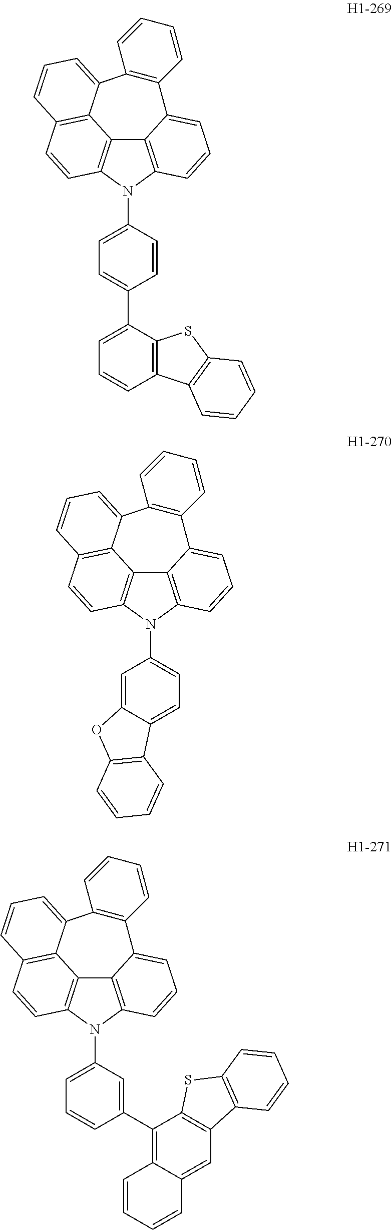

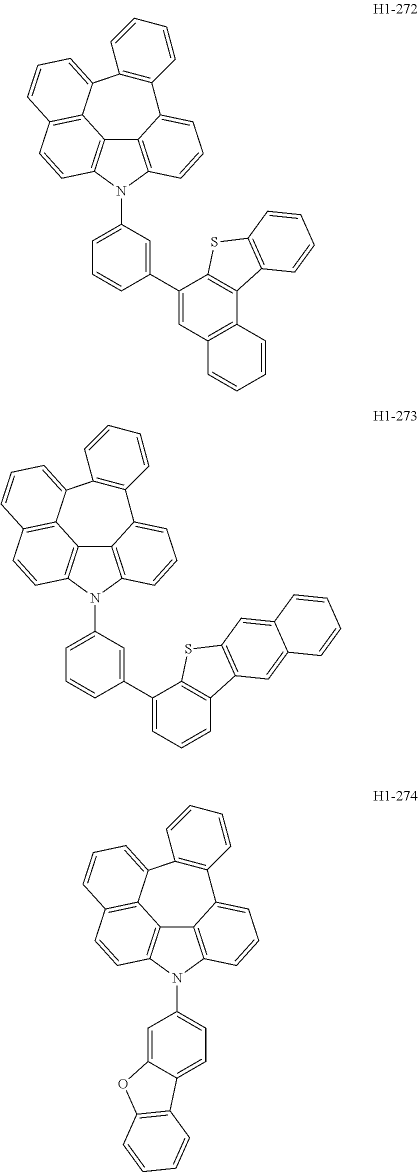 [SMILES: c1cc(-c2cc3ccccc3c3c2sc2ccccc23)cc(-n2c3cccc4c3c3c5c(cccc5ccc32)-c2ccccc2-4)c1 ; c1cc(-c2cccc3c2sc2cc4ccccc4cc23)cc(-n2c3cccc4c3c3c5c(cccc5ccc32)-c2ccccc2-4)c1 ; c1ccc2c(c1)-c1cccc3ccc4c(c13)c1c-2cccc1n4-c1ccc2c(c1)oc1ccccc12]

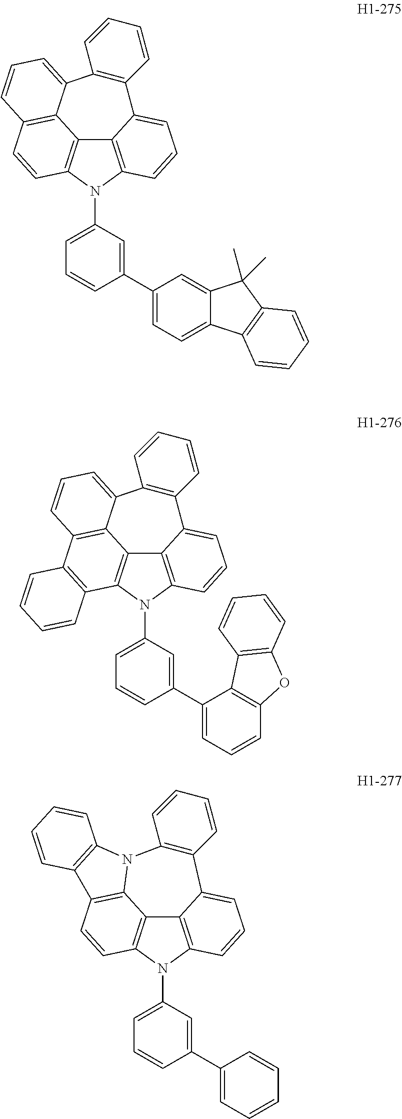 [SMILES: CC1(C)c2ccccc2-c2ccc(-c3cccc(-n4c5cccc6c5c5c7c(cccc7ccc54)-c4ccccc4-6)c3)cc21 ; c1cc(-c2cccc3oc4ccccc4c23)cc(-n2c3cccc4c3c3c5c(cccc5c5ccccc5c32)-c2ccccc2-4)c1 ; c1ccc(-c2cccc(-n3c4cccc5c6ccccc6n6c7ccccc7c7ccc3c(c54)c76)c2)cc1]

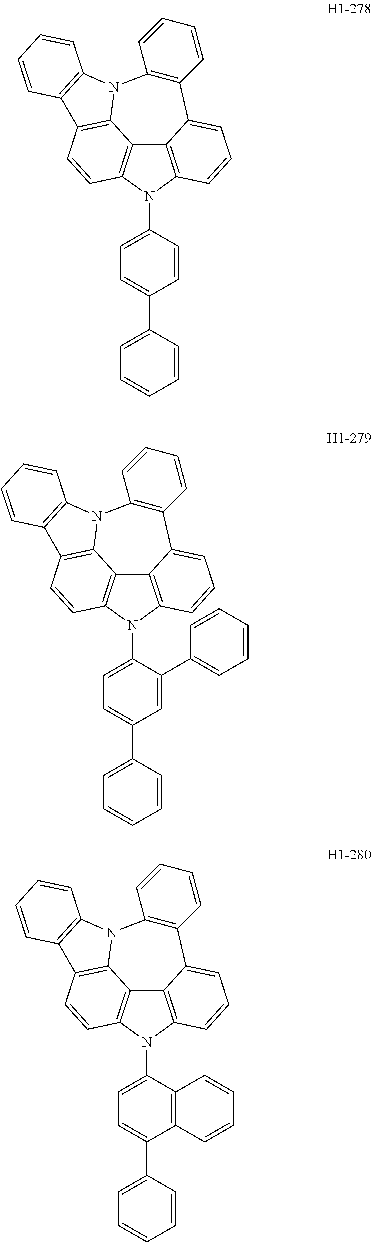 [SMILES: c1ccc(-c2ccc(-n3c4cccc5c6ccccc6n6c7ccccc7c7ccc3c(c54)c76)cc2)cc1 ; c1ccc(-c2ccc(-n3c4cccc5c6ccccc6n6c7ccccc7c7ccc3c(c54)c76)c(-c3ccccc3)c2)cc1 ; c1ccc(-c2ccc(-n3c4cccc5c6ccccc6n6c7ccccc7c7ccc3c(c54)c76)c3ccccc23)cc1]

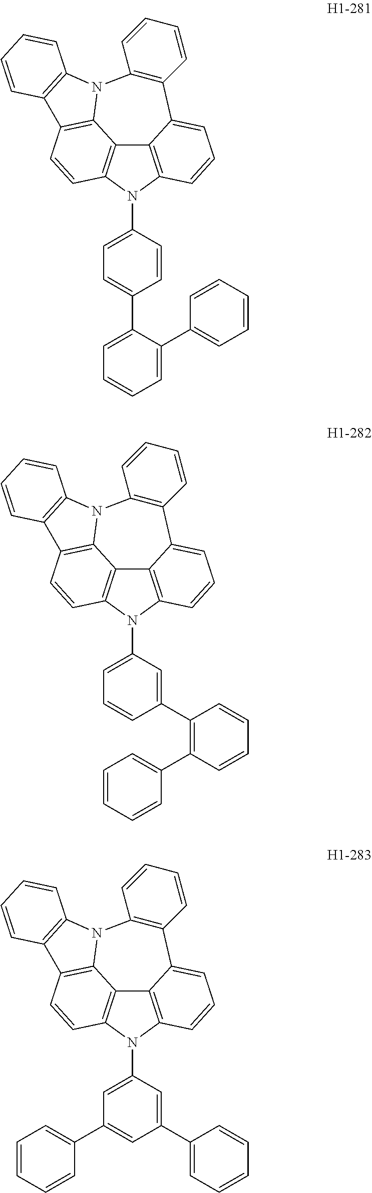

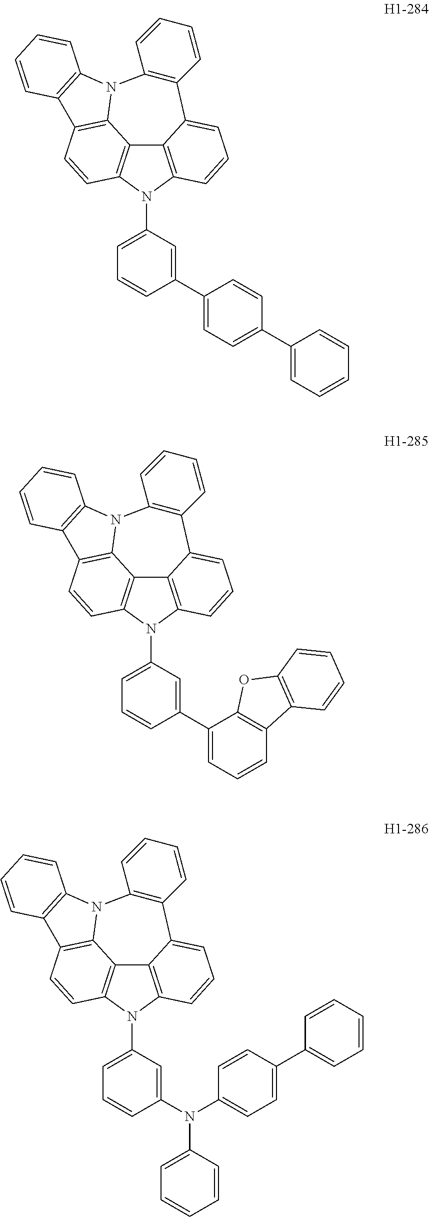 [SMILES: c1ccc(-c2ccc(-c3cccc(-n4c5cccc6c7ccccc7n7c8ccccc8c8ccc4c(c65)c87)c3)cc2)cc1 ; c1cc(-c2cccc3c2oc2ccccc23)cc(-n2c3cccc4c5ccccc5n5c6ccccc6c6ccc2c(c43)c65)c1 ; c1ccc(-c2ccc(N(c3ccccc3)c3cccc(-n4c5cccc6c7ccccc7n7c8ccccc8c8ccc4c(c65)c87)c3)cc2)cc1]

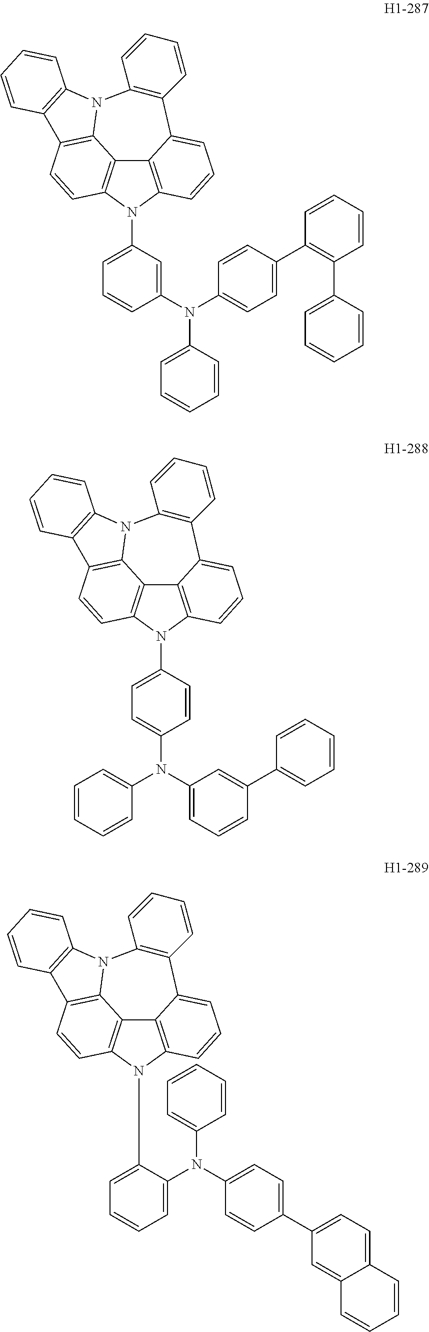 [SMILES: c1ccc(-c2ccccc2-c2ccc(N(c3ccccc3)c3cccc(-n4c5cccc6c7ccccc7n7c8ccccc8c8ccc4c(c65)c87)c3)cc2)cc1 ; c1ccc(-c2cccc(N(c3ccccc3)c3ccc(-n4c5cccc6c7ccccc7n7c8ccccc8c8ccc4c(c65)c87)cc3)c2)cc1 ; c1ccc(N(c2ccc(-c3ccc4ccccc4c3)cc2)c2ccccc2-n2c3cccc4c5ccccc5n5c6ccccc6c6ccc2c(c43)c65)cc1]

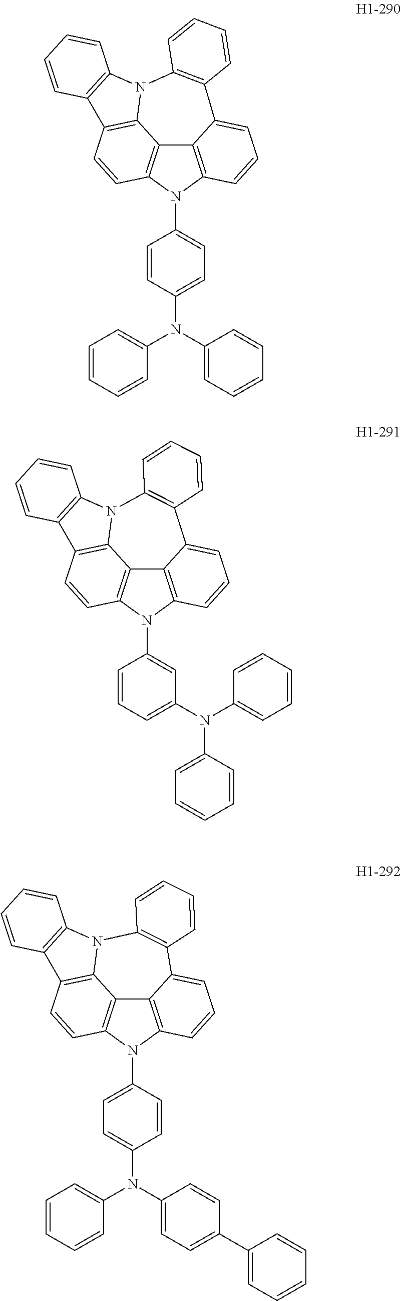 [SMILES: c1ccc(N(c2ccccc2)c2ccc(-n3c4cccc5c6ccccc6n6c7ccccc7c7ccc3c(c54)c76)cc2)cc1 ; c1ccc(N(c2ccccc2)c2cccc(-n3c4cccc5c6ccccc6n6c7ccccc7c7ccc3c(c54)c76)c2)cc1 ; c1ccc(-c2ccc(N(c3ccccc3)c3ccc(-n4c5cccc6c7ccccc7n7c8ccccc8c8ccc4c(c65)c87)cc3)cc2)cc1]

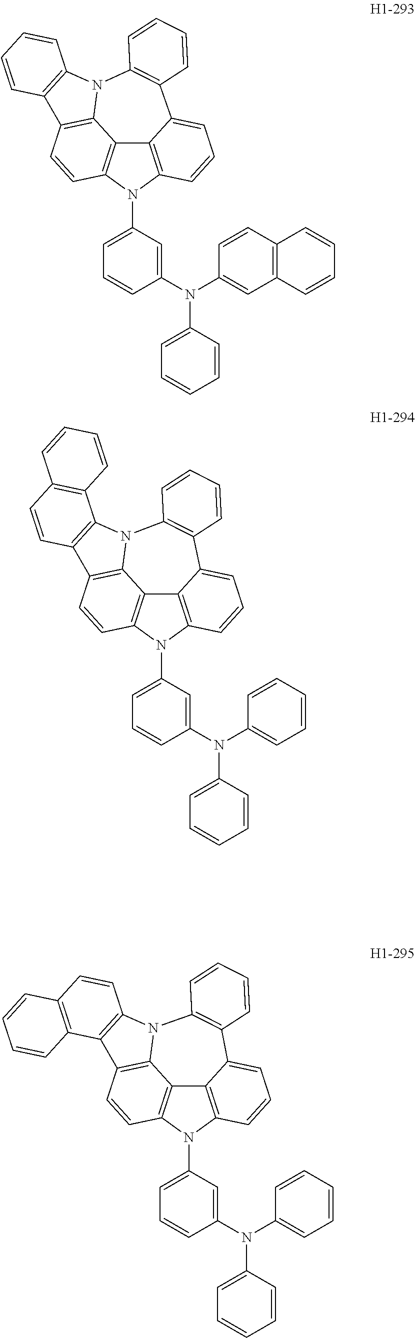 [SMILES: c1ccc(N(c2cccc(-n3c4cccc5c6ccccc6n6c7ccccc7c7ccc3c(c54)c76)c2)c2ccc3ccccc3c2)cc1 ; c1ccc(N(c2ccccc2)c2cccc(-n3c4cccc5c6ccccc6n6c7c8ccccc8ccc7c7ccc3c(c54)c76)c2)cc1 ; c1ccc(N(c2ccccc2)c2cccc(-n3c4cccc5c6ccccc6n6c7ccc8ccccc8c7c7ccc3c(c54)c76)c2)cc1]

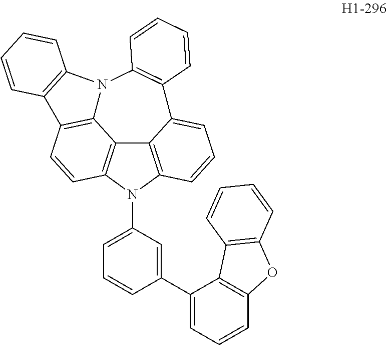 [SMILES: c1cc(-c2cccc3oc4ccccc4c23)cc(-n2c3cccc4c5ccccc5n5c6ccccc6c6ccc2c(c43)c65)c1]

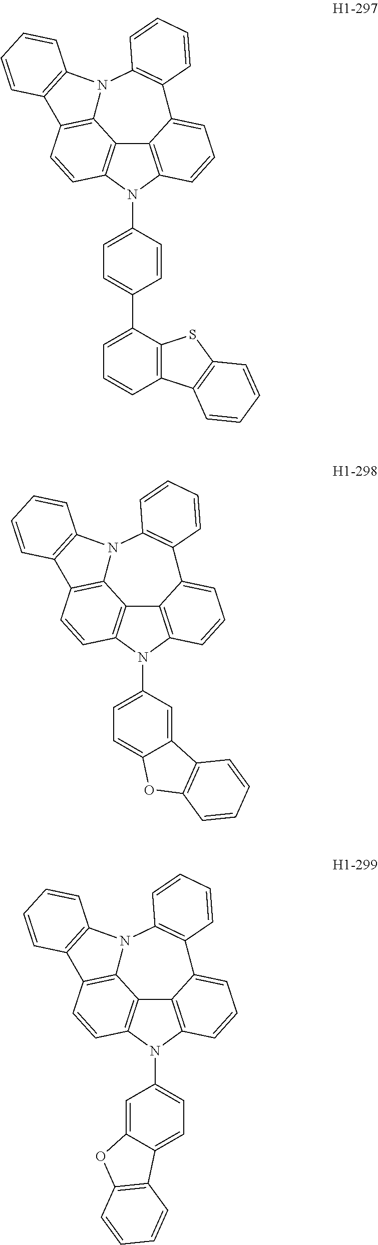 [SMILES: c1ccc2c(c1)sc1c(-c3ccc(-n4c5cccc6c7ccccc7n7c8ccccc8c8ccc4c(c65)c87)cc3)cccc12 ; c1ccc2c(c1)oc1ccc(-n3c4cccc5c6ccccc6n6c7ccccc7c7ccc3c(c54)c76)cc12 ; c1ccc2c(c1)oc1cc(-n3c4cccc5c6ccccc6n6c7ccccc7c7ccc3c(c54)c76)ccc12]

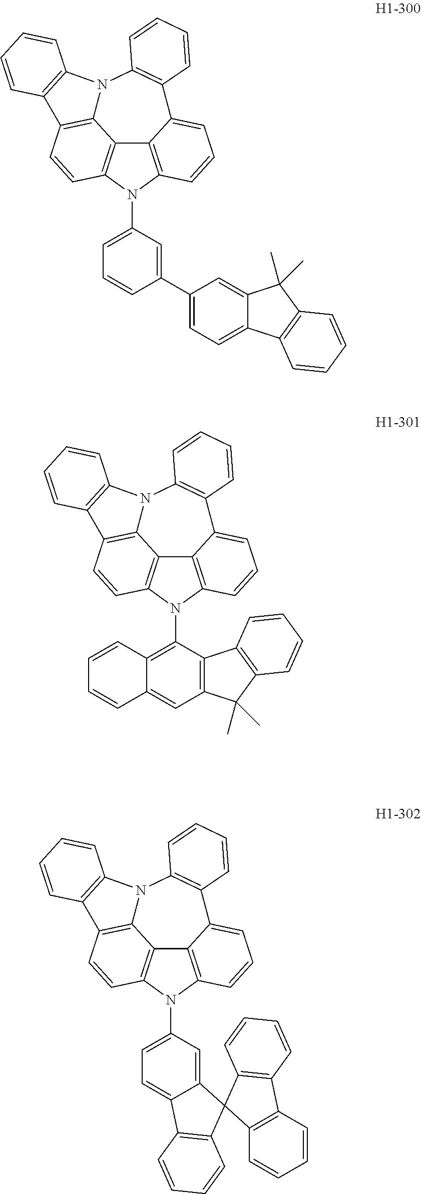 [SMILES: CC1(C)c2ccccc2-c2ccc(-c3cccc(-n4c5cccc6c7ccccc7n7c8ccccc8c8ccc4c(c65)c87)c3)cc21 ; CC1(C)c2ccccc2-c2c1cc1ccccc1c2-n1c2cccc3c4ccccc4n4c5ccccc5c5ccc1c(c32)c54 ; c1ccc2c(c1)-c1ccccc1C21c2ccccc2-c2ccc(-n3c4cccc5c6ccccc6n6c7ccccc7c7ccc3c(c54)c76)cc21]

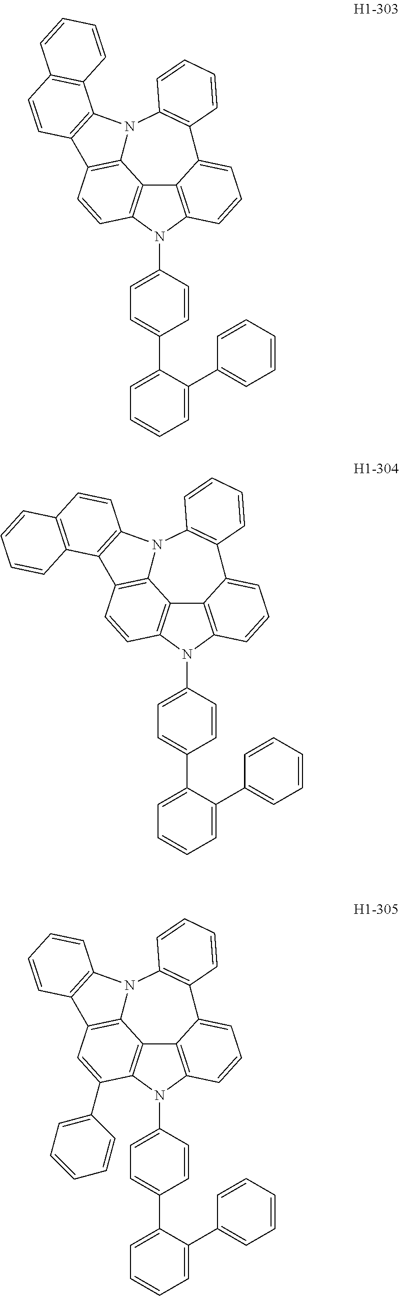 [SMILES: c1ccc(-c2ccccc2-c2ccc(-n3c4cccc5c6ccccc6n6c7c8ccccc8ccc7c7ccc3c(c54)c76)cc2)cc1 ; c1ccc(-c2ccccc2-c2ccc(-n3c4cccc5c6ccccc6n6c7ccc8ccccc8c7c7ccc3c(c54)c76)cc2)cc1 ; c1ccc(-c2ccccc2-c2ccc(-n3c4cccc5c6ccccc6n6c7ccccc7c7cc(-c8ccccc8)c3c(c54)c76)cc2)cc1]

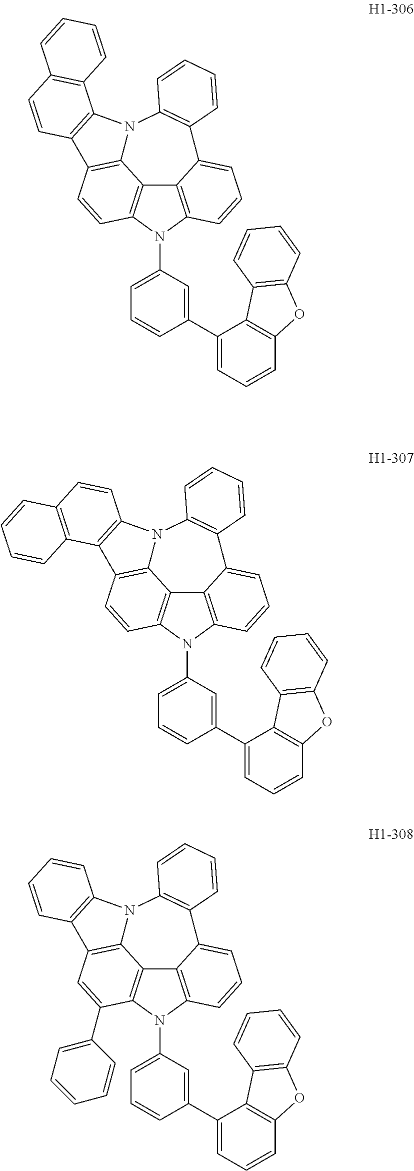 [SMILES: c1cc(-c2cccc3oc4ccccc4c23)cc(-n2c3cccc4c5ccccc5n5c6c7ccccc7ccc6c6ccc2c(c43)c65)c1 ; c1cc(-c2cccc3oc4ccccc4c23)cc(-n2c3cccc4c5ccccc5n5c6ccc7ccccc7c6c6ccc2c(c43)c65)c1 ; c1ccc(-c2cc3c4ccccc4n4c5ccccc5c5cccc6c5c(c2n6-c2cccc(-c5cccc6oc7ccccc7c56)c2)c34)cc1]

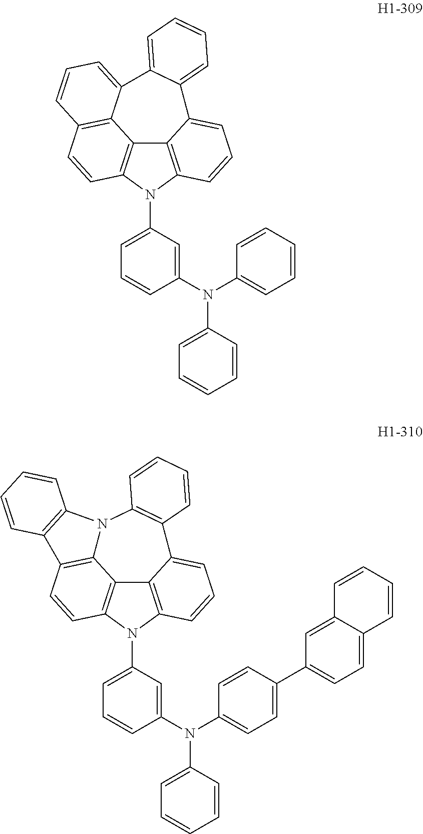 [SMILES: c1ccc(N(c2ccccc2)c2cccc(-n3c4cccc5c4c4c6c(cccc6ccc43)-c3ccccc3-5)c2)cc1 ; c1ccc(N(c2ccc(-c3ccc4ccccc4c3)cc2)c2cccc(-n3c4cccc5c6ccccc6n6c7ccccc7c7ccc3c(c54)c76)c2)cc1]

The compound represented by any one of formulas 2 to 4 may be at least one selected from the group consisting of the following compounds, but is not limited thereto.

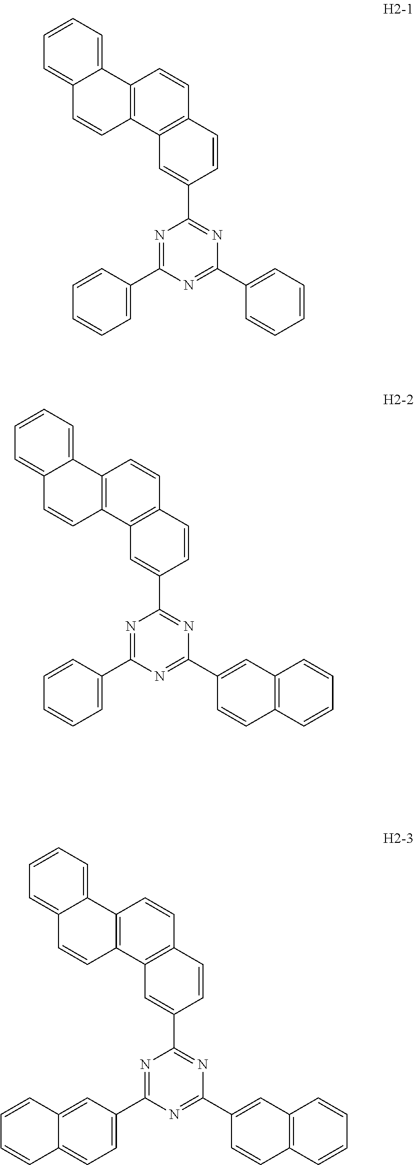 [SMILES: c1ccc(-c2nc(-c3ccccc3)nc(-c3ccc4ccc5c6ccccc6ccc5c4c3)n2)cc1 ; c1ccc(-c2nc(-c3ccc4ccccc4c3)nc(-c3ccc4ccc5c6ccccc6ccc5c4c3)n2)cc1 ; c1ccc2cc(-c3nc(-c4ccc5ccccc5c4)nc(-c4ccc5ccc6c7ccccc7ccc6c5c4)n3)ccc2c1]

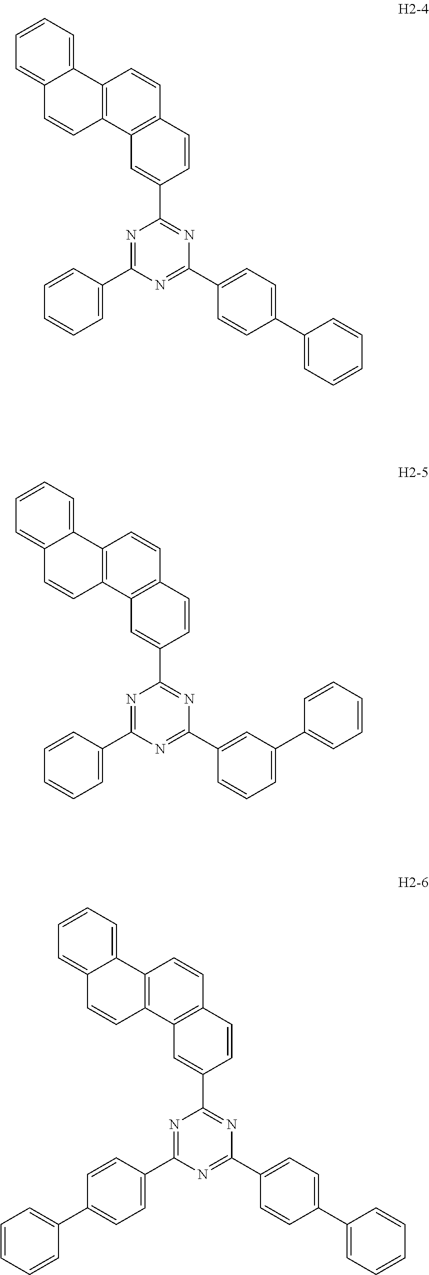 [SMILES: c1ccc(-c2ccc(-c3nc(-c4ccccc4)nc(-c4ccc5ccc6c7ccccc7ccc6c5c4)n3)cc2)cc1 ; c1ccc(-c2cccc(-c3nc(-c4ccccc4)nc(-c4ccc5ccc6c7ccccc7ccc6c5c4)n3)c2)cc1 ; c1ccc(-c2ccc(-c3nc(-c4ccc(-c5ccccc5)cc4)nc(-c4ccc5ccc6c7ccccc7ccc6c5c4)n3)cc2)cc1]

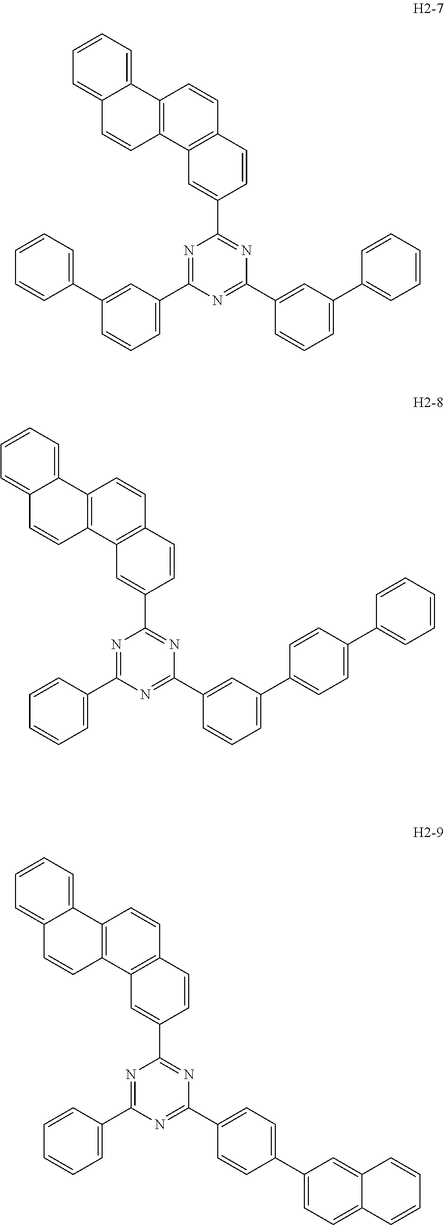 [SMILES: c1ccc(-c2cccc(-c3nc(-c4cccc(-c5ccccc5)c4)nc(-c4ccc5ccc6c7ccccc7ccc6c5c4)n3)c2)cc1 ; c1ccc(-c2ccc(-c3cccc(-c4nc(-c5ccccc5)nc(-c5ccc6ccc7c8ccccc8ccc7c6c5)n4)c3)cc2)cc1 ; c1ccc(-c2nc(-c3ccc(-c4ccc5ccccc5c4)cc3)nc(-c3ccc4ccc5c6ccccc6ccc5c4c3)n2)cc1]

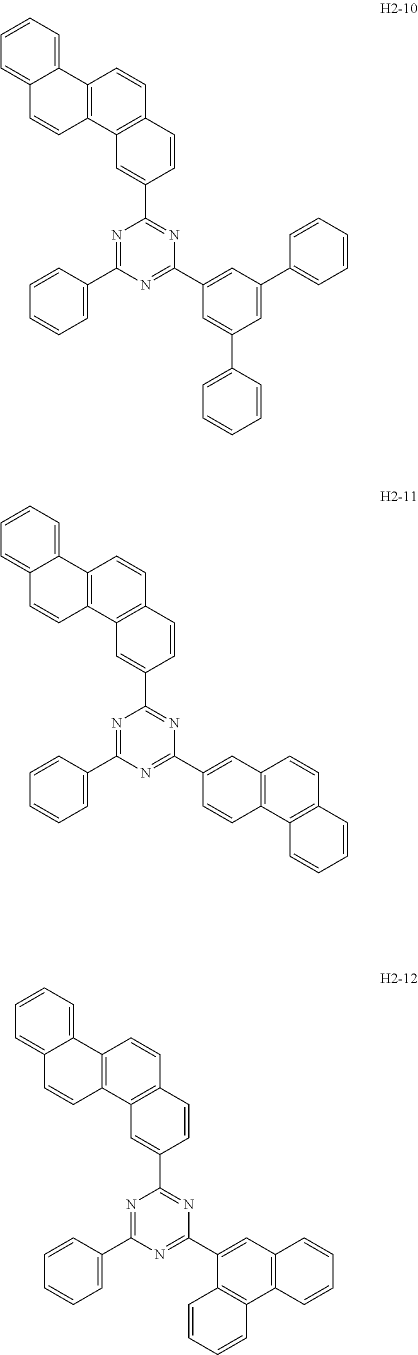 [SMILES: c1ccc(-c2cc(-c3ccccc3)cc(-c3nc(-c4ccccc4)nc(-c4ccc5ccc6c7ccccc7ccc6c5c4)n3)c2)cc1 ; c1ccc(-c2nc(-c3ccc4c(ccc5ccccc54)c3)nc(-c3ccc4ccc5c6ccccc6ccc5c4c3)n2)cc1 ; c1ccc(-c2nc(-c3ccc4ccc5c6ccccc6ccc5c4c3)nc(-c3cc4ccccc4c4ccccc34)n2)cc1]

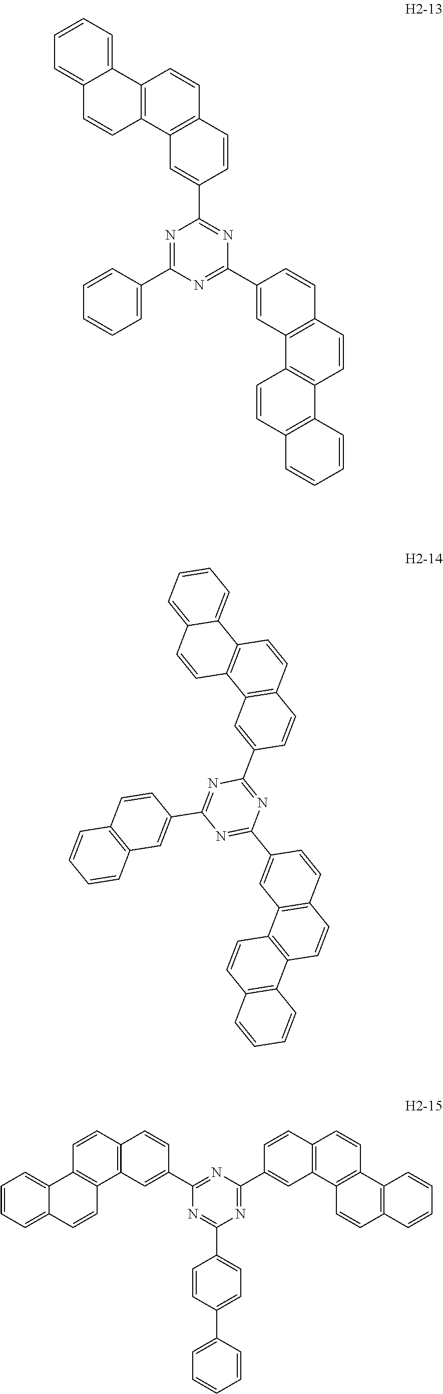 [SMILES: c1ccc(-c2nc(-c3ccc4ccc5c6ccccc6ccc5c4c3)nc(-c3ccc4ccc5c6ccccc6ccc5c4c3)n2)cc1 ; c1ccc2cc(-c3nc(-c4ccc5ccc6c7ccccc7ccc6c5c4)nc(-c4ccc5ccc6c7ccccc7ccc6c5c4)n3)ccc2c1 ; c1ccc(-c2ccc(-c3nc(-c4ccc5ccc6c7ccccc7ccc6c5c4)nc(-c4ccc5ccc6c7ccccc7ccc6c5c4)n3)cc2)cc1]

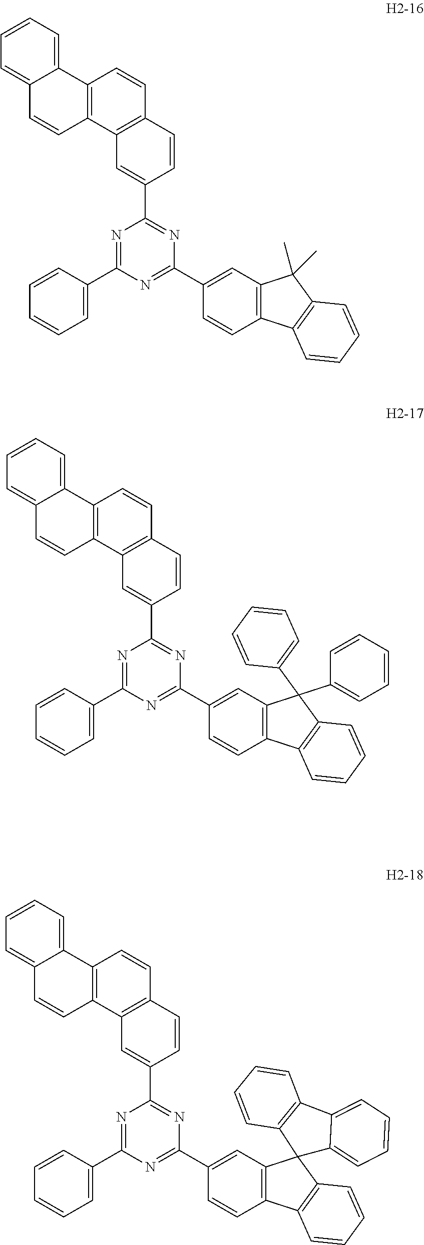 [SMILES: CC1(C)c2ccccc2-c2ccc(-c3nc(-c4ccccc4)nc(-c4ccc5ccc6c7ccccc7ccc6c5c4)n3)cc21 ; c1ccc(-c2nc(-c3ccc4c(c3)C(c3ccccc3)(c3ccccc3)c3ccccc3-4)nc(-c3ccc4ccc5c6ccccc6ccc5c4c3)n2)cc1 ; c1ccc(-c2nc(-c3ccc4c(c3)C3(c5ccccc5-c5ccccc53)c3ccccc3-4)nc(-c3ccc4ccc5c6ccccc6ccc5c4c3)n2)cc1]

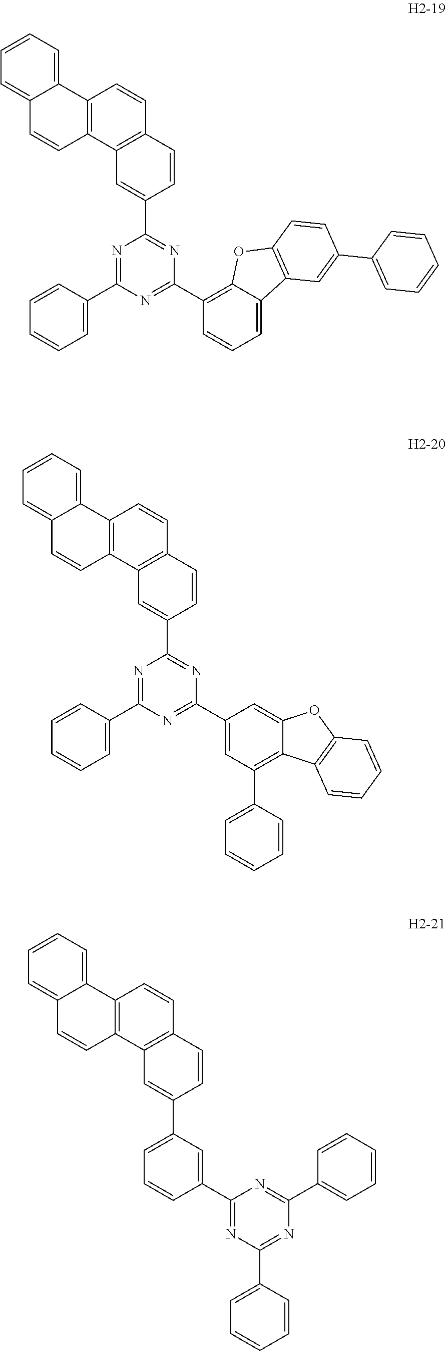 [SMILES: c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccccc3)nc(-c3cccc(-c4ccc5ccc6c7ccccc7ccc6c5c4)c3)n2)cc1]

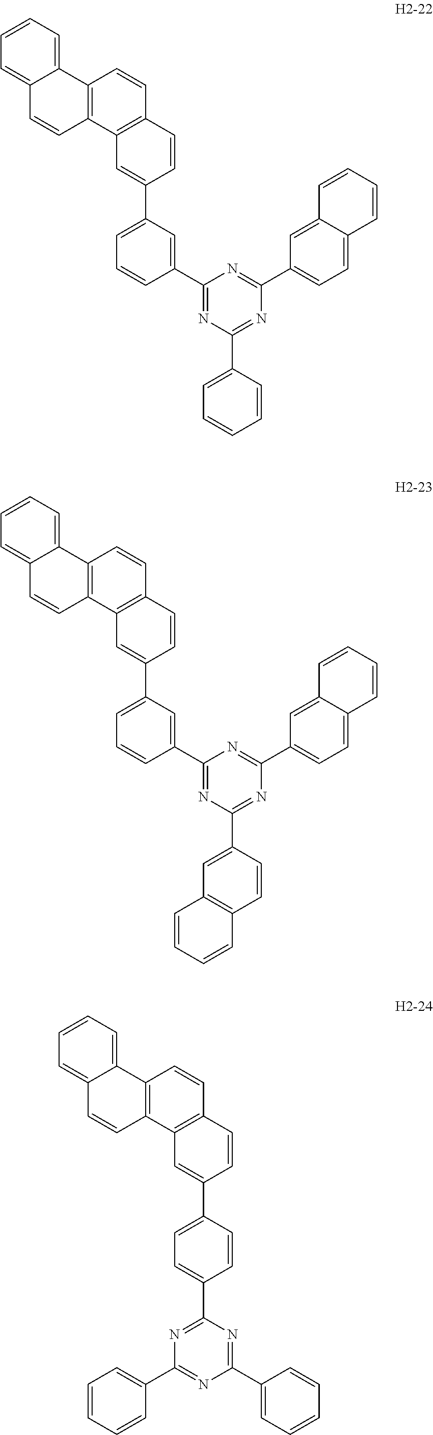 [SMILES: c1ccc(-c2nc(-c3cccc(-c4ccc5ccc6c7ccccc7ccc6c5c4)c3)nc(-c3ccc4ccccc4c3)n2)cc1 ; c1cc(-c2ccc3ccc4c5ccccc5ccc4c3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc6c7ccccc7ccc6c5c4)cc3)n2)cc1]

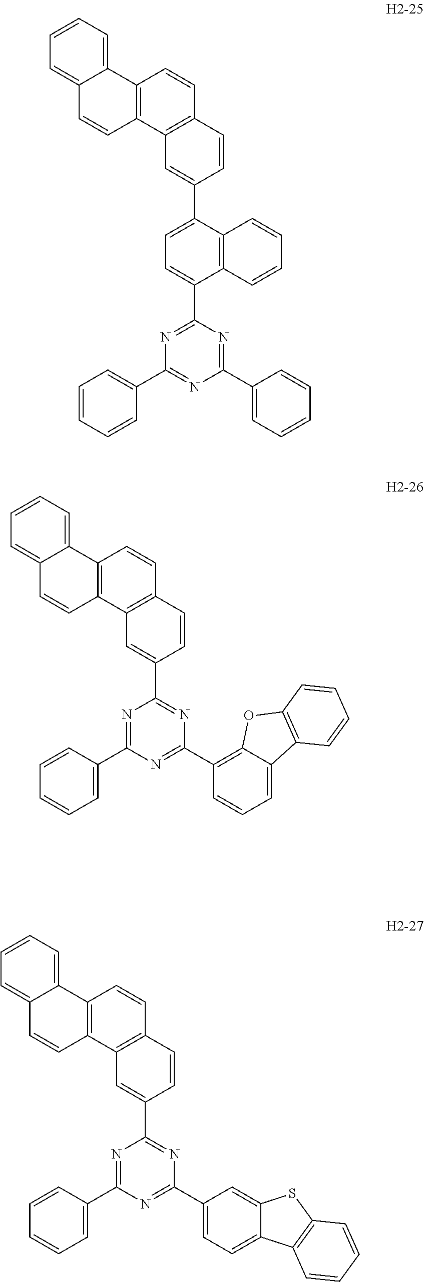 [SMILES: c1ccc(-c2nc(-c3ccccc3)nc(-c3ccc(-c4ccc5ccc6c7ccccc7ccc6c5c4)c4ccccc34)n2)cc1 ; c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1]

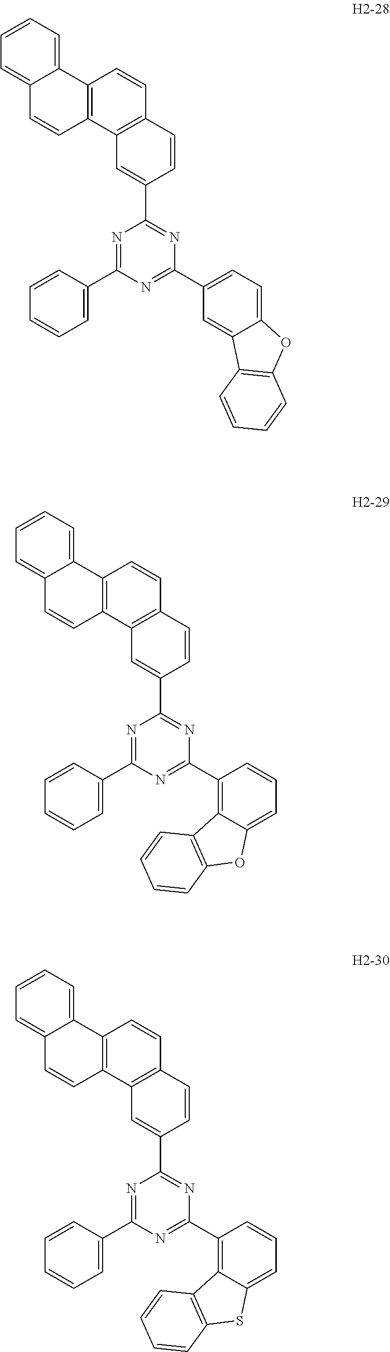 [SMILES: c1ccc(-c2nc(-c3ccc4ccc5c6ccccc6ccc5c4c3)nc(-c3ccc4oc5ccccc5c4c3)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1]

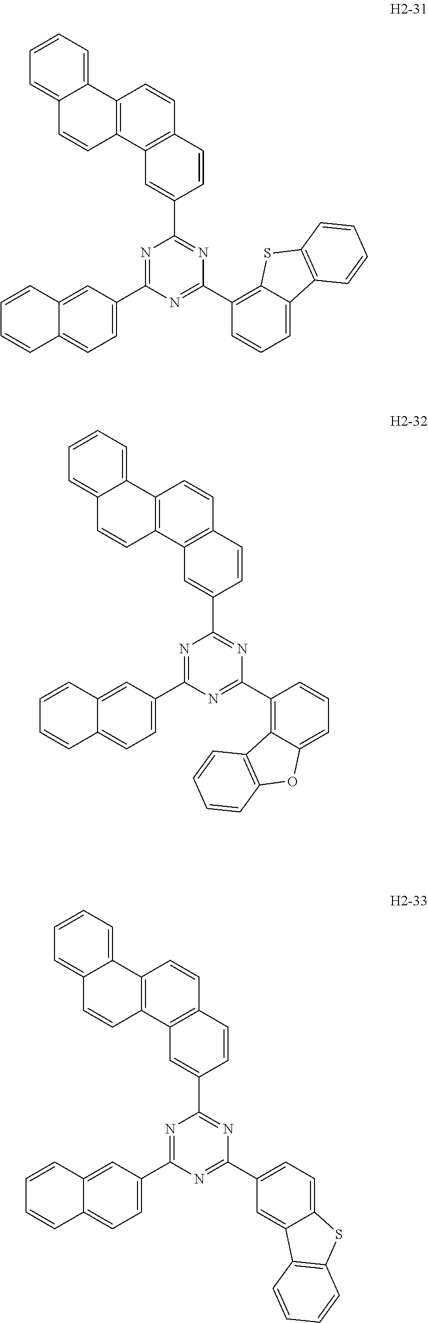 [SMILES: c1ccc2cc(-c3nc(-c4ccc5ccc6c7ccccc7ccc6c5c4)nc(-c4cccc5c4sc4ccccc45)n3)ccc2c1 ; c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1]

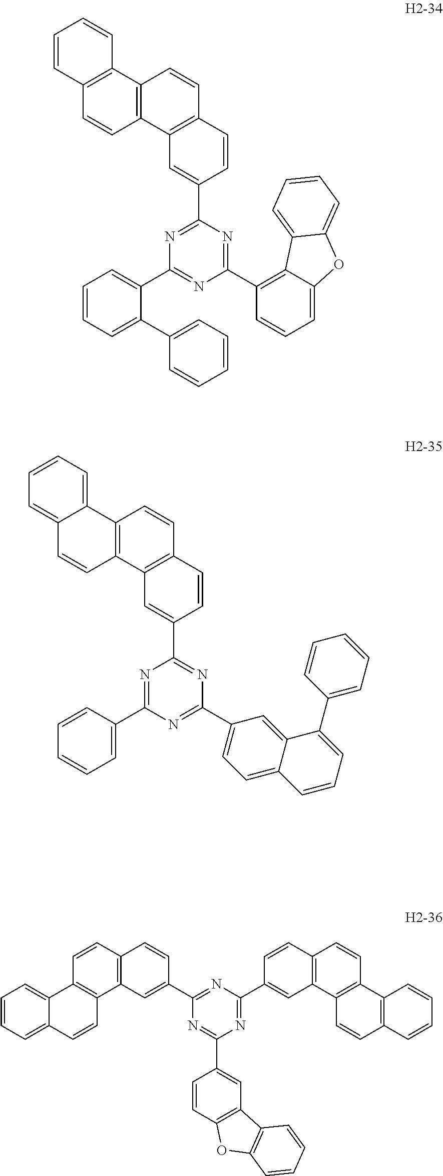 [SMILES: c1ccc(-c2ccccc2-c2nc(-c3ccc4ccc5c6ccccc6ccc5c4c3)nc(-c3cccc4oc5ccccc5c34)n2)cc1 ; c1ccc(-c2nc(-c3ccc4cccc(-c5ccccc5)c4c3)nc(-c3ccc4ccc5c6ccccc6ccc5c4c3)n2)cc1 ; c1ccc2c(c1)ccc1c3cc(-c4nc(-c5ccc6ccc7c8ccccc8ccc7c6c5)nc(-c5ccc6oc7ccccc7c6c5)n4)ccc3ccc21]

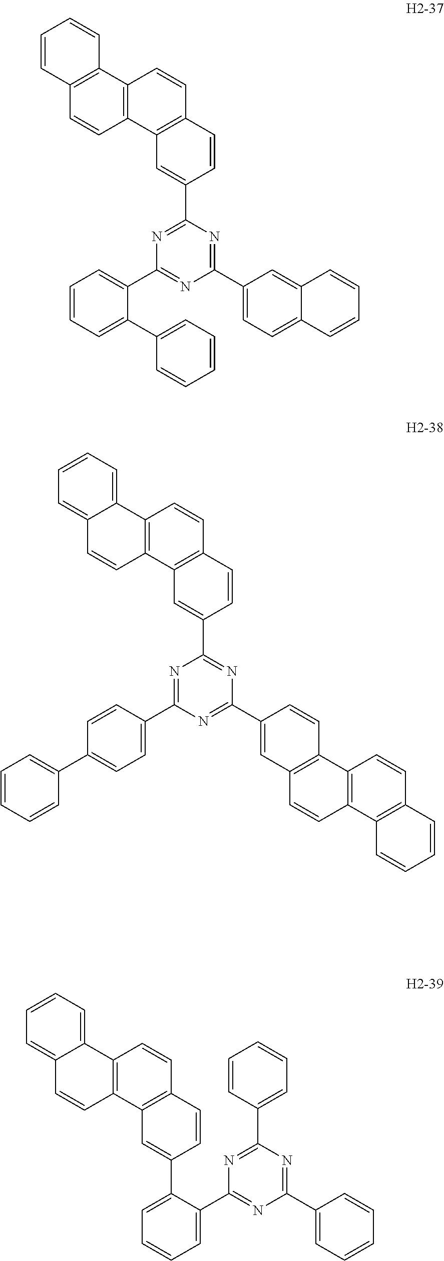 [SMILES: c1ccc(-c2ccccc2-c2nc(-c3ccc4ccccc4c3)nc(-c3ccc4ccc5c6ccccc6ccc5c4c3)n2)cc1 ; c1ccc(-c2ccc(-c3nc(-c4ccc5c(ccc6c7ccccc7ccc56)c4)nc(-c4ccc5ccc6c7ccccc7ccc6c5c4)n3)cc2)cc1 ; c1ccc(-c2nc(-c3ccccc3)nc(-c3ccccc3-c3ccc4ccc5c6ccccc6ccc5c4c3)n2)cc1]

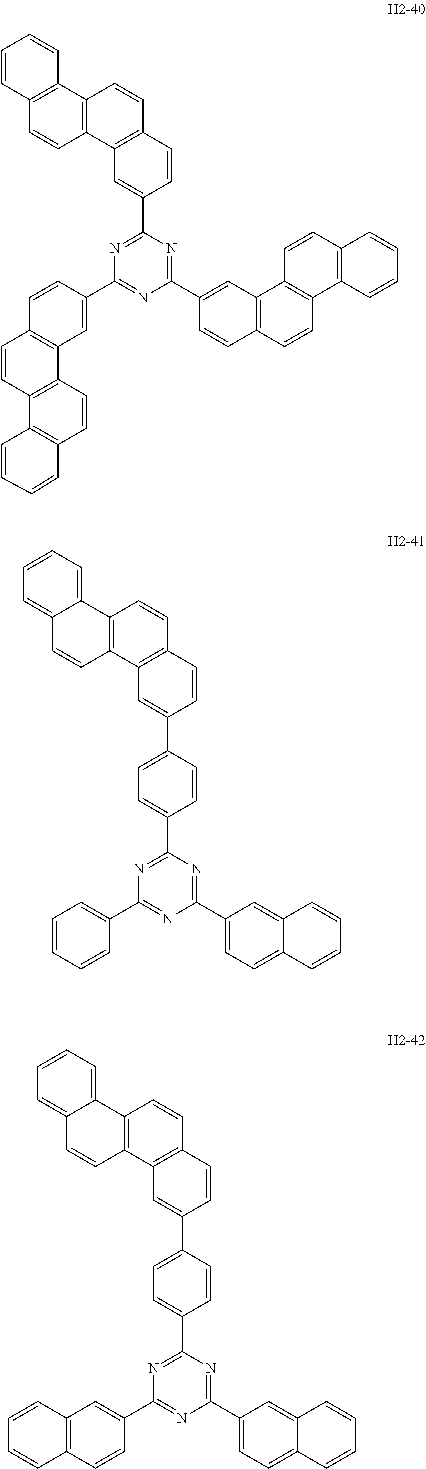 [SMILES: c1ccc2c(c1)ccc1c3cc(-c4nc(-c5ccc6ccc7c8ccccc8ccc7c6c5)nc(-c5ccc6ccc7c8ccccc8ccc7c6c5)n4)ccc3ccc21 ; c1ccc(-c2nc(-c3ccc(-c4ccc5ccc6c7ccccc7ccc6c5c4)cc3)nc(-c3ccc4ccccc4c3)n2)cc1 ; c1ccc2cc(-c3nc(-c4ccc(-c5ccc6ccc7c8ccccc8ccc7c6c5)cc4)nc(-c4ccc5ccccc5c4)n3)ccc2c1]

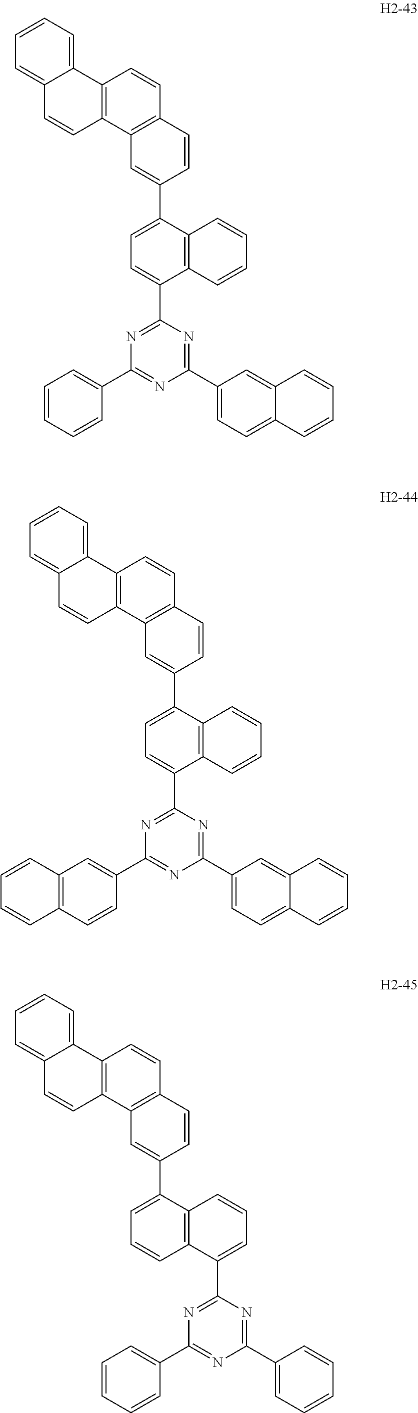 [SMILES: c1ccc(-c2nc(-c3ccc4ccccc4c3)nc(-c3ccc(-c4ccc5ccc6c7ccccc7ccc6c5c4)c4ccccc34)n2)cc1 ; c1ccc2cc(-c3nc(-c4ccc5ccccc5c4)nc(-c4ccc(-c5ccc6ccc7c8ccccc8ccc7c6c5)c5ccccc45)n3)ccc2c1 ; c1ccc(-c2nc(-c3ccccc3)nc(-c3cccc4c(-c5ccc6ccc7c8ccccc8ccc7c6c5)cccc34)n2)cc1]

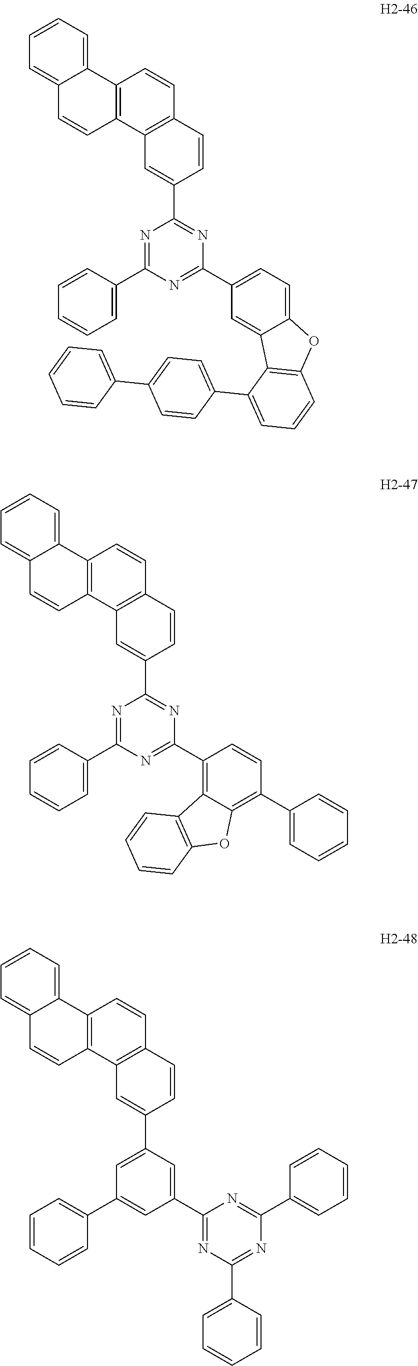 [SMILES: c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1 ; c1ccc(-c2nc(-c3ccc4ccc5c6ccccc6ccc5c4c3)nc(-c3ccc(-c4ccccc4)c4oc5ccccc5c34)n2)cc1 ; c1ccc(-c2cc(-c3ccc4ccc5c6ccccc6ccc5c4c3)cc(-c3nc(-c4ccccc4)nc(-c4ccccc4)n3)c2)cc1]

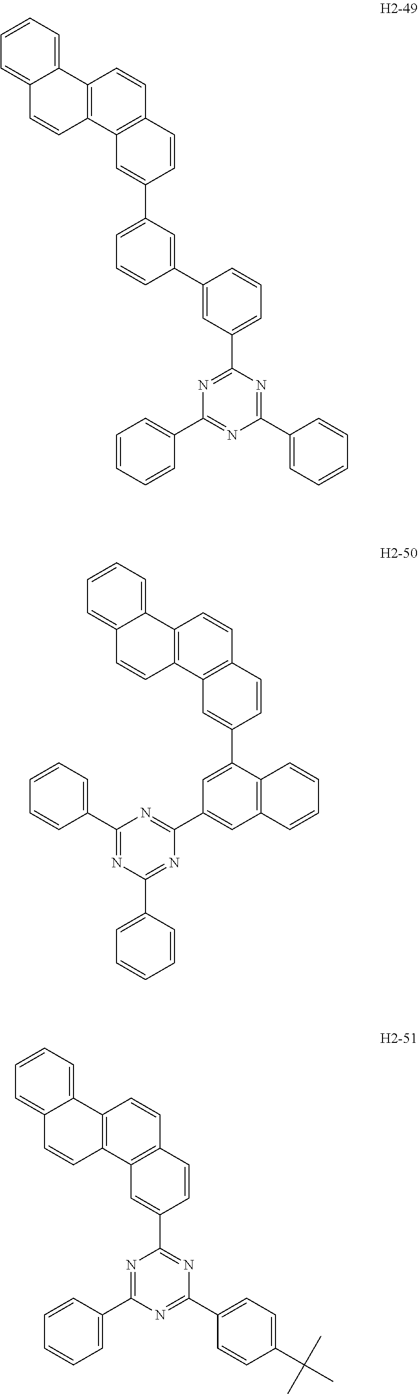 [SMILES: c1ccc(-c2nc(-c3ccccc3)nc(-c3cccc(-c4cccc(-c5ccc6ccc7c8ccccc8ccc7c6c5)c4)c3)n2)cc1 ; c1ccc(-c2nc(-c3ccccc3)nc(-c3cc(-c4ccc5ccc6c7ccccc7ccc6c5c4)c4ccccc4c3)n2)cc1 ; CC(C)(C)c1ccc(-c2nc(-c3ccccc3)nc(-c3ccc4ccc5c6ccccc6ccc5c4c3)n2)cc1]

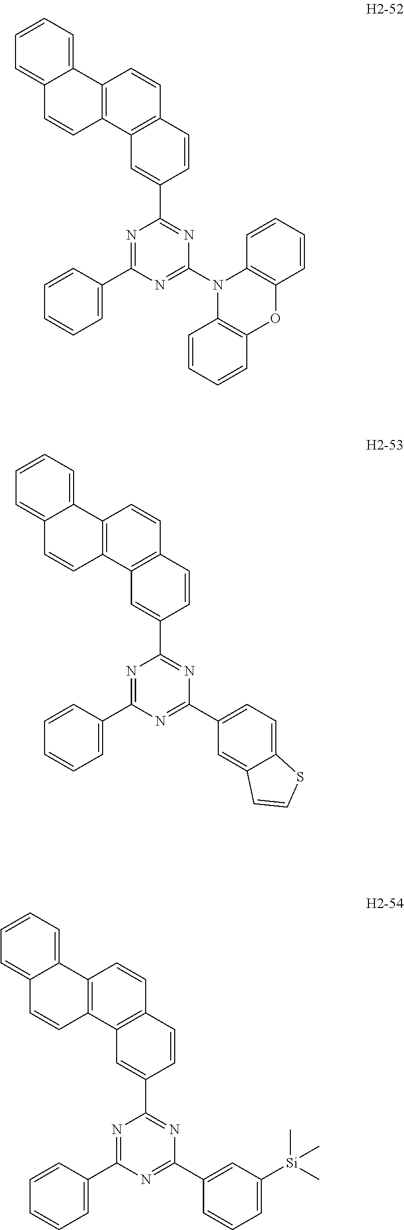 [SMILES: c1ccc(-c2nc(-c3ccc4ccc5c6ccccc6ccc5c4c3)nc(N3c4ccccc4Oc4ccccc43)n2)cc1 ; c1ccc(-c2nc(-c3ccc4sccc4c3)nc(-c3ccc4ccc5c6ccccc6ccc5c4c3)n2)cc1 ; C[Si](C)(C)c1cccc(-c2nc(-c3ccccc3)nc(-c3ccc4ccc5c6ccccc6ccc5c4c3)n2)c1]

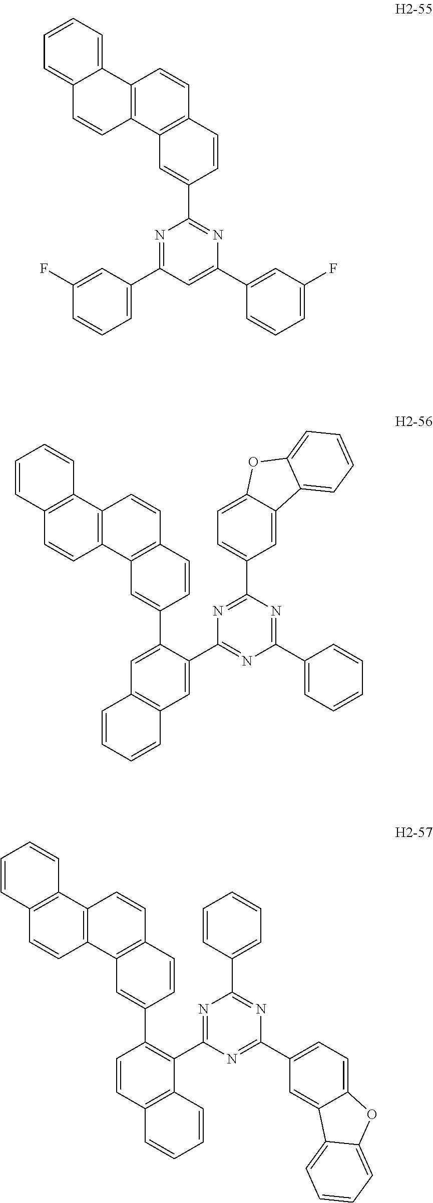 [SMILES: Fc1cccc(-c2cc(-c3cccc(F)c3)nc(-c3ccc4ccc5c6ccccc6ccc5c4c3)n2)c1 ; c1ccc(-c2nc(-c3ccc4oc5ccccc5c4c3)nc(-c3cc4ccccc4cc3-c3ccc4ccc5c6ccccc6ccc5c4c3)n2)cc1 ; c1ccc(-c2nc(-c3ccc4oc5ccccc5c4c3)nc(-c3c(-c4ccc5ccc6c7ccccc7ccc6c5c4)ccc4ccccc34)n2)cc1]

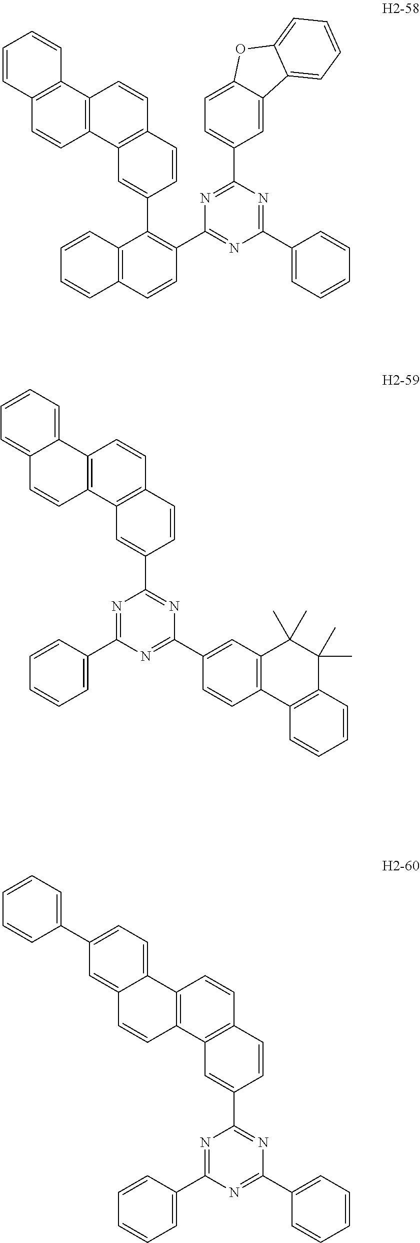 [SMILES: c1ccc(-c2nc(-c3ccc4oc5ccccc5c4c3)nc(-c3ccc4ccccc4c3-c3ccc4ccc5c6ccccc6ccc5c4c3)n2)cc1 ; CC1(C)c2ccccc2-c2ccc(-c3nc(-c4ccccc4)nc(-c4ccc5ccc6c7ccccc7ccc6c5c4)n3)cc2C1(C)C ; c1ccc(-c2ccc3c(ccc4c5cc(-c6nc(-c7ccccc7)nc(-c7ccccc7)n6)ccc5ccc34)c2)cc1]

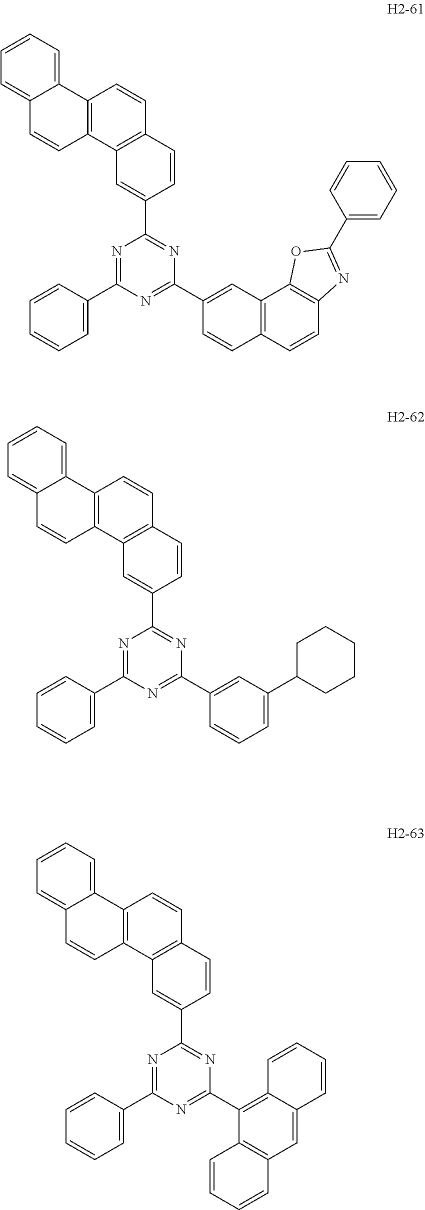 [SMILES: c1ccc(-c2nc(-c3ccc4ccc5c6ccccc6ccc5c4c3)nc(-c3ccc4ccc5nc(-c6ccccc6)oc5c4c3)n2)cc1 ; c1ccc(-c2nc(-c3cccc(C4CCCCC4)c3)nc(-c3ccc4ccc5c6ccccc6ccc5c4c3)n2)cc1 ; c1ccc(-c2nc(-c3ccc4ccc5c6ccccc6ccc5c4c3)nc(-c3c4ccccc4cc4ccccc34)n2)cc1]

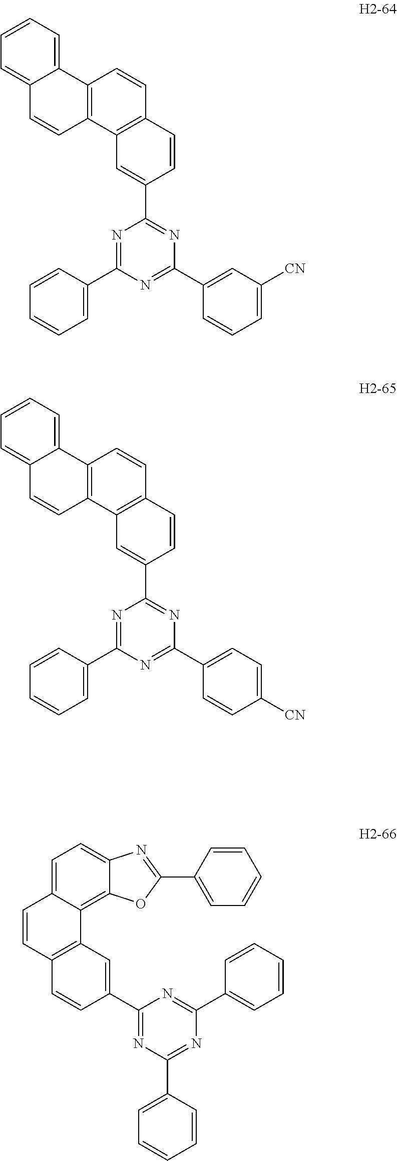 [SMILES: N#Cc1cccc(-c2nc(-c3ccccc3)nc(-c3ccc4ccc5c6ccccc6ccc5c4c3)n2)c1 ; N#Cc1ccc(-c2nc(-c3ccccc3)nc(-c3ccc4ccc5c6ccccc6ccc5c4c3)n2)cc1 ; c1ccc(-c2nc(-c3ccccc3)nc(-c3ccc4ccc5ccc6nc(-c7ccccc7)oc6c5c4c3)n2)cc1]

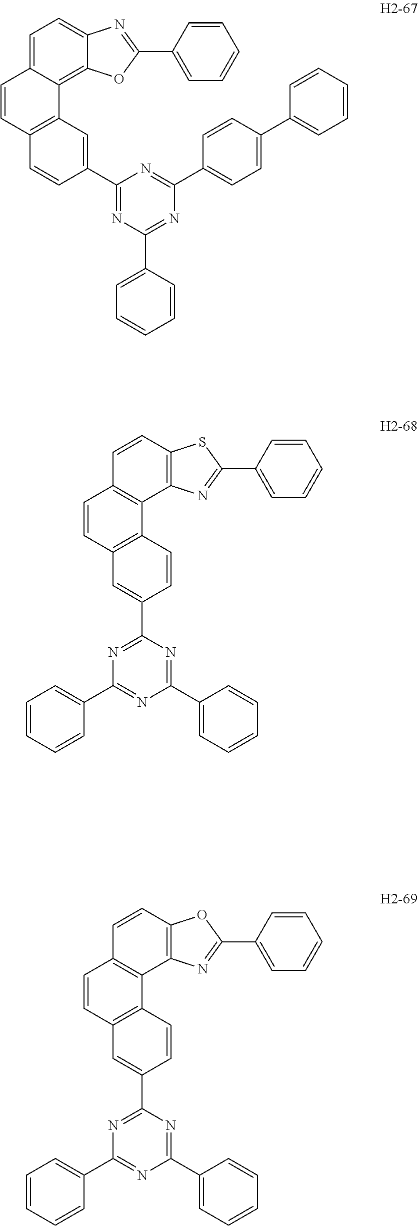 [SMILES: c1ccc(-c2ccc(-c3nc(-c4ccccc4)nc(-c4ccc5ccc6ccc7nc(-c8ccccc8)oc7c6c5c4)n3)cc2)cc1 ; c1ccc(-c2nc(-c3ccccc3)nc(-c3ccc4c(ccc5ccc6sc(-c7ccccc7)nc6c54)c3)n2)cc1 ; c1ccc(-c2nc(-c3ccccc3)nc(-c3ccc4c(ccc5ccc6oc(-c7ccccc7)nc6c54)c3)n2)cc1]

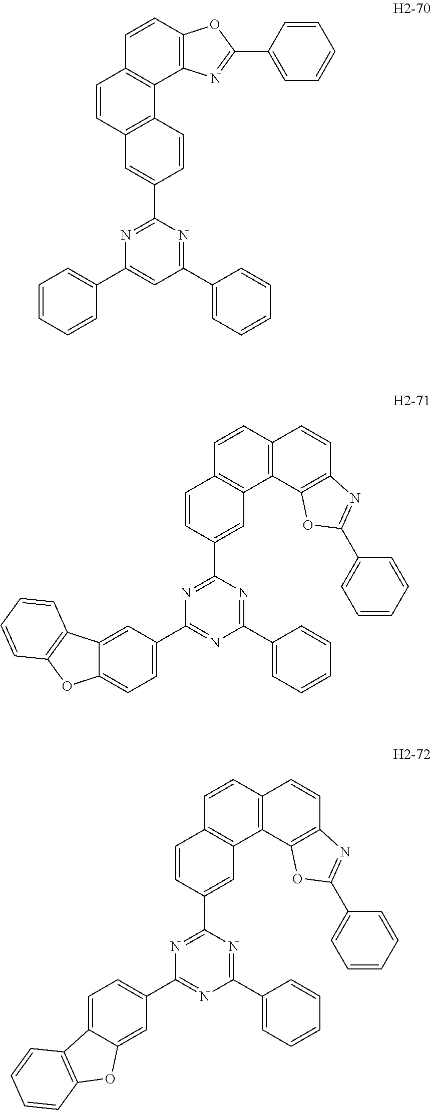 [SMILES: c1ccc(-c2cc(-c3ccccc3)nc(-c3ccc4c(ccc5ccc6oc(-c7ccccc7)nc6c54)c3)n2)cc1 ; c1ccc(-c2nc(-c3ccc4oc5ccccc5c4c3)nc(-c3ccc4ccc5ccc6nc(-c7ccccc7)oc6c5c4c3)n2)cc1 ; c1ccc(-c2nc(-c3ccc4c(c3)oc3ccccc34)nc(-c3ccc4ccc5ccc6nc(-c7ccccc7)oc6c5c4c3)n2)cc1]

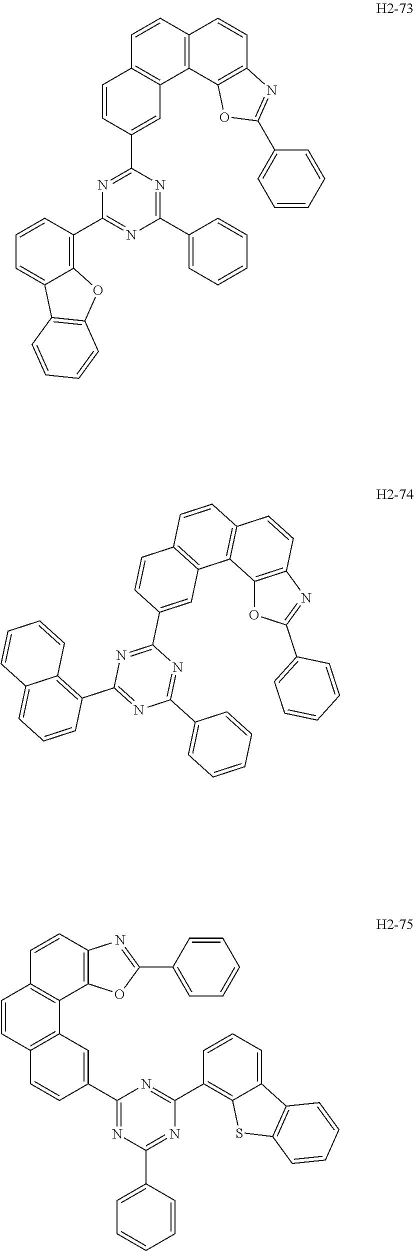 [SMILES: c1ccc(-c2nc(-c3ccc4ccc5ccc6nc(-c7ccccc7)oc6c5c4c3)nc(-c3cccc4c3oc3ccccc34)n2)cc1 ; c1ccc(-c2nc(-c3ccc4ccc5ccc6nc(-c7ccccc7)oc6c5c4c3)nc(-c3cccc4ccccc34)n2)cc1 ; c1ccc(-c2nc(-c3ccc4ccc5ccc6nc(-c7ccccc7)oc6c5c4c3)nc(-c3cccc4c3sc3ccccc34)n2)cc1]

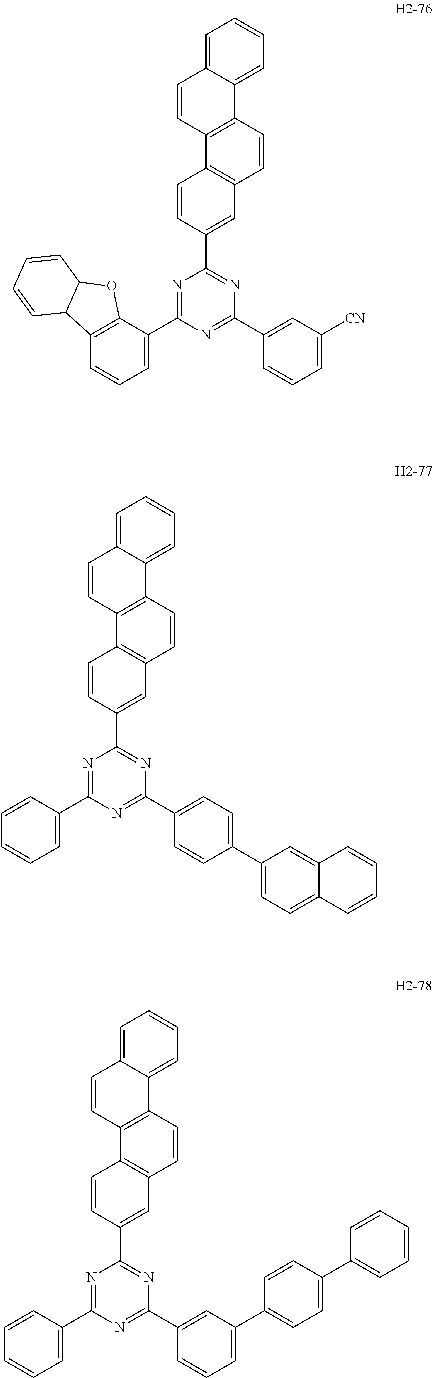 [SMILES: N#Cc1cccc(-c2nc(-c3ccc4c(ccc5c6ccccc6ccc45)c3)nc(-c3cccc4c3OC3C=CC=CC43)n2)c1 ; c1ccc(-c2nc(-c3ccc(-c4ccc5ccccc5c4)cc3)nc(-c3ccc4c(ccc5c6ccccc6ccc45)c3)n2)cc1 ; c1ccc(-c2ccc(-c3cccc(-c4nc(-c5ccccc5)nc(-c5ccc6c(ccc7c8ccccc8ccc67)c5)n4)c3)cc2)cc1]

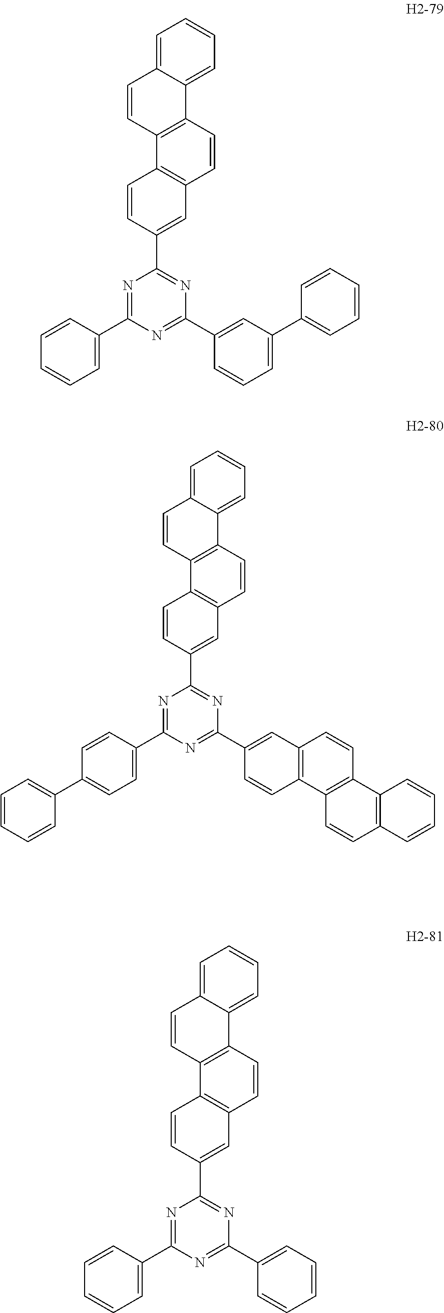 [SMILES: c1ccc(-c2cccc(-c3nc(-c4ccccc4)nc(-c4ccc5c(ccc6c7ccccc7ccc56)c4)n3)c2)cc1 ; c1ccc(-c2ccc(-c3nc(-c4ccc5c(ccc6c7ccccc7ccc56)c4)nc(-c4ccc5c(ccc6c7ccccc7ccc56)c4)n3)cc2)cc1 ; c1ccc(-c2nc(-c3ccccc3)nc(-c3ccc4c(ccc5c6ccccc6ccc45)c3)n2)cc1]

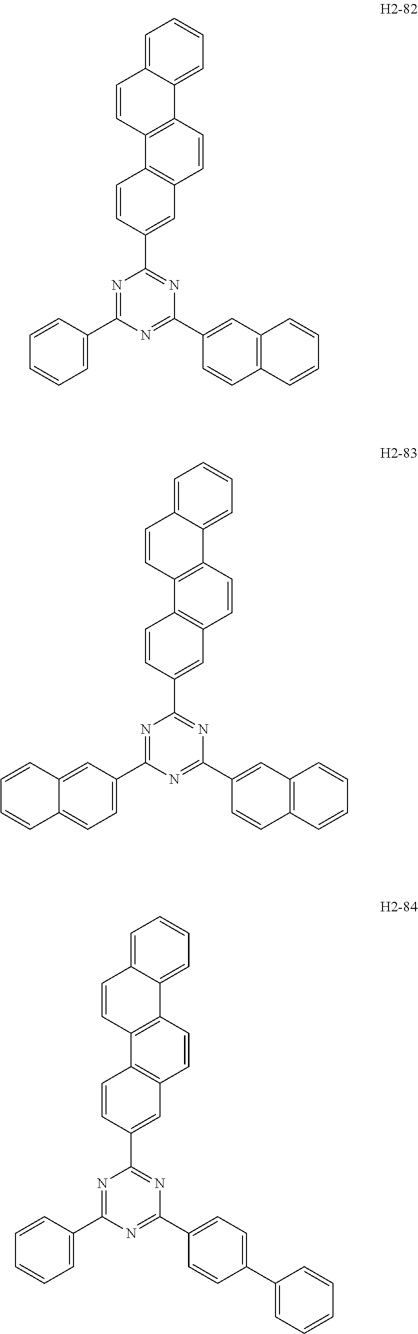 [SMILES: c1ccc(-c2nc(-c3ccc4ccccc4c3)nc(-c3ccc4c(ccc5c6ccccc6ccc45)c3)n2)cc1 ; c1ccc2cc(-c3nc(-c4ccc5ccccc5c4)nc(-c4ccc5c(ccc6c7ccccc7ccc56)c4)n3)ccc2c1 ; c1ccc(-c2ccc(-c3nc(-c4ccccc4)nc(-c4ccc5c(ccc6c7ccccc7ccc56)c4)n3)cc2)cc1]

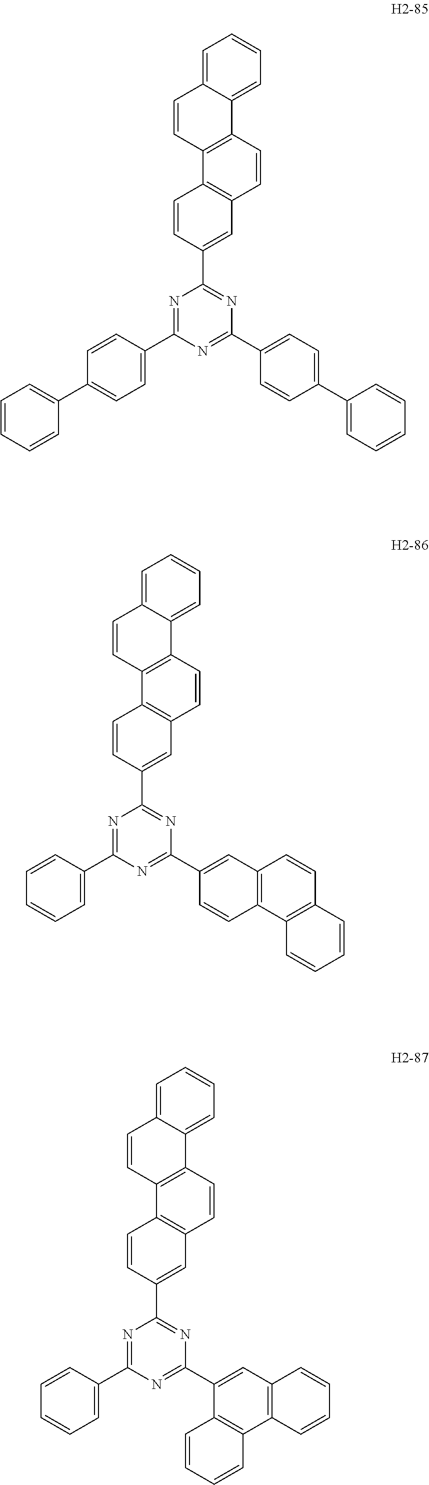 [SMILES: c1ccc(-c2ccc(-c3nc(-c4ccc(-c5ccccc5)cc4)nc(-c4ccc5c(ccc6c7ccccc7ccc56)c4)n3)cc2)cc1 ; c1ccc(-c2nc(-c3ccc4c(ccc5ccccc54)c3)nc(-c3ccc4c(ccc5c6ccccc6ccc45)c3)n2)cc1 ; c1ccc(-c2nc(-c3ccc4c(ccc5c6ccccc6ccc45)c3)nc(-c3cc4ccccc4c4ccccc34)n2)cc1]

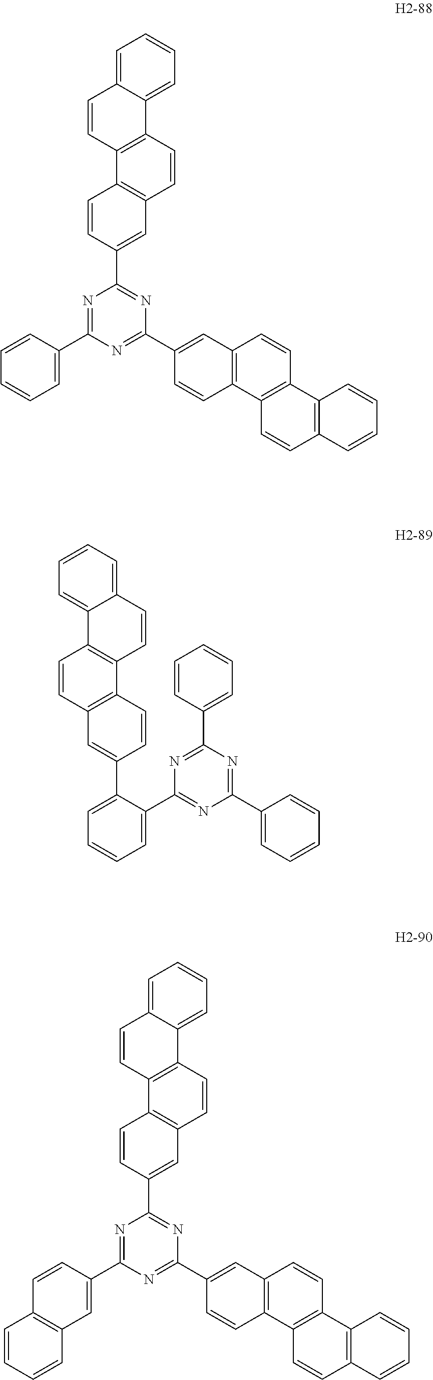 [SMILES: c1ccc(-c2nc(-c3ccc4c(ccc5c6ccccc6ccc45)c3)nc(-c3ccc4c(ccc5c6ccccc6ccc45)c3)n2)cc1 ; c1ccc(-c2nc(-c3ccccc3)nc(-c3ccccc3-c3ccc4c(ccc5c6ccccc6ccc45)c3)n2)cc1 ; c1ccc2cc(-c3nc(-c4ccc5c(ccc6c7ccccc7ccc56)c4)nc(-c4ccc5c(ccc6c7ccccc7ccc56)c4)n3)ccc2c1]

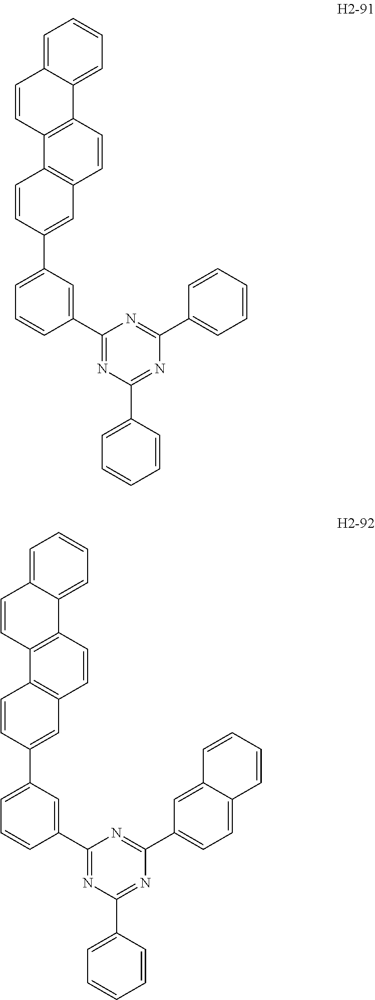 [SMILES: c1ccc(-c2nc(-c3ccccc3)nc(-c3cccc(-c4ccc5c(ccc6c7ccccc7ccc56)c4)c3)n2)cc1 ; c1ccc(-c2nc(-c3cccc(-c4ccc5c(ccc6c7ccccc7ccc56)c4)c3)nc(-c3ccc4ccccc4c3)n2)cc1]

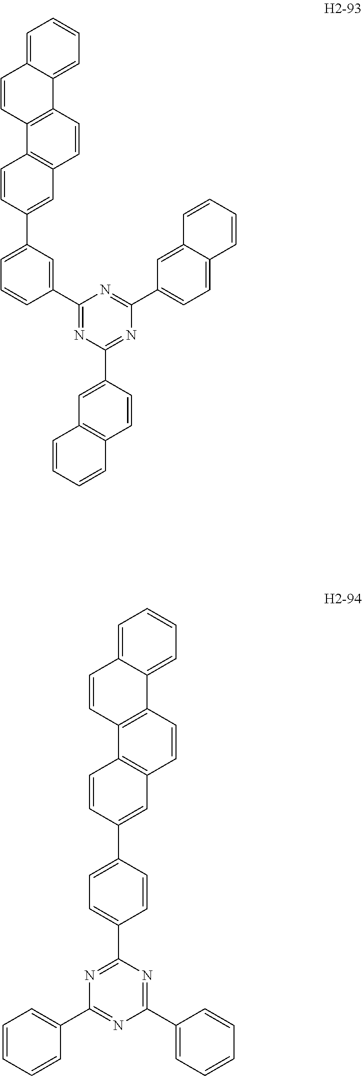 [SMILES: c1cc(-c2ccc3c(ccc4c5ccccc5ccc34)c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5c(ccc6c7ccccc7ccc56)c4)cc3)n2)cc1]

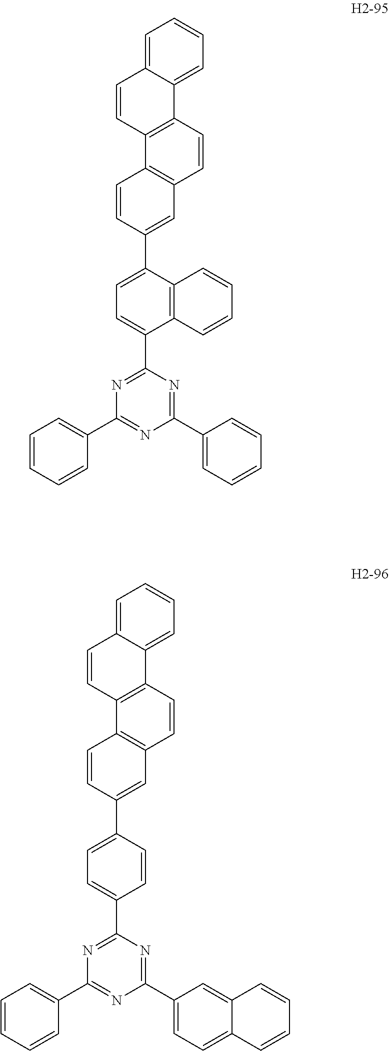 [SMILES: c1ccc(-c2nc(-c3ccccc3)nc(-c3ccc(-c4ccc5c(ccc6c7ccccc7ccc56)c4)c4ccccc34)n2)cc1 ; c1ccc(-c2nc(-c3ccc(-c4ccc5c(ccc6c7ccccc7ccc56)c4)cc3)nc(-c3ccc4ccccc4c3)n2)cc1]

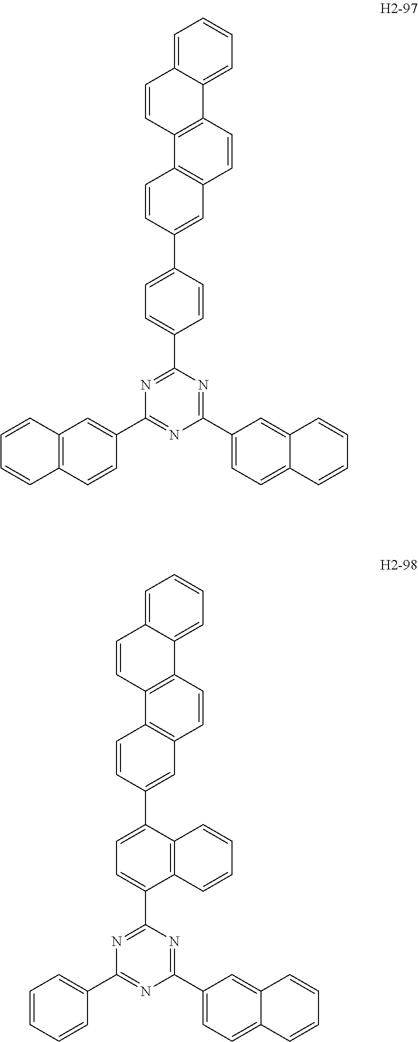 [SMILES: c1ccc2cc(-c3nc(-c4ccc(-c5ccc6c(ccc7c8ccccc8ccc67)c5)cc4)nc(-c4ccc5ccccc5c4)n3)ccc2c1 ; c1ccc(-c2nc(-c3ccc4ccccc4c3)nc(-c3ccc(-c4ccc5c(ccc6c7ccccc7ccc56)c4)c4ccccc34)n2)cc1]

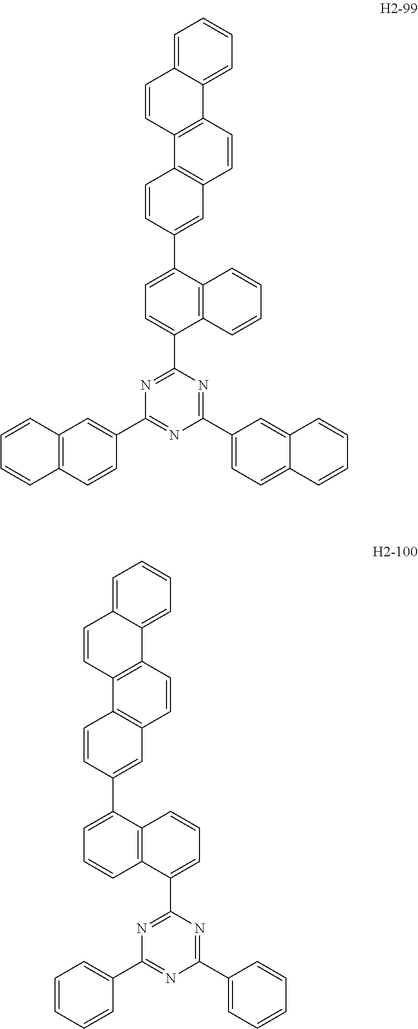 [SMILES: c1ccc2cc(-c3nc(-c4ccc5ccccc5c4)nc(-c4ccc(-c5ccc6c(ccc7c8ccccc8ccc67)c5)c5ccccc45)n3)ccc2c1 ; c1ccc(-c2nc(-c3ccccc3)nc(-c3cccc4c(-c5ccc6c(ccc7c8ccccc8ccc67)c5)cccc34)n2)cc1]

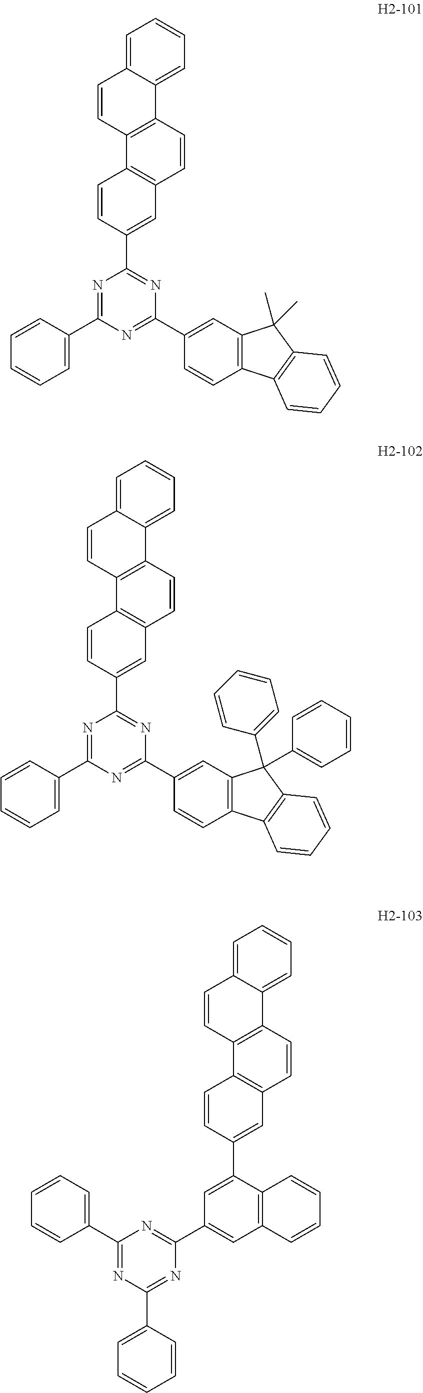 [SMILES: CC1(C)c2ccccc2-c2ccc(-c3nc(-c4ccccc4)nc(-c4ccc5c(ccc6c7ccccc7ccc56)c4)n3)cc21 ; c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1 ; c1ccc(-c2nc(-c3ccccc3)nc(-c3cc(-c4ccc5c(ccc6c7ccccc7ccc56)c4)c4ccccc4c3)n2)cc1]

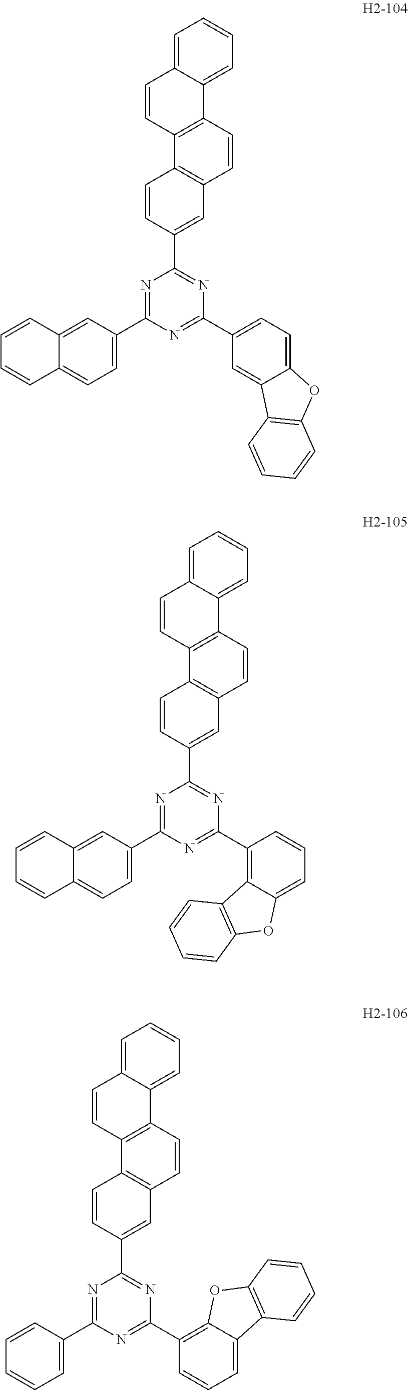 [SMILES: c1ccc2cc(-c3nc(-c4ccc5c(ccc6c7ccccc7ccc56)c4)nc(-c4ccc5oc6ccccc6c5c4)n3)ccc2c1 ; c1ccc2cc(-c3nc(-c4ccc5c(ccc6c7ccccc7ccc56)c4)nc(-c4cccc5oc6ccccc6c45)n3)ccc2c1 ; c1ccc(-c2nc(-c3ccc4c(ccc5c6ccccc6ccc45)c3)nc(-c3cccc4c3oc3ccccc34)n2)cc1]

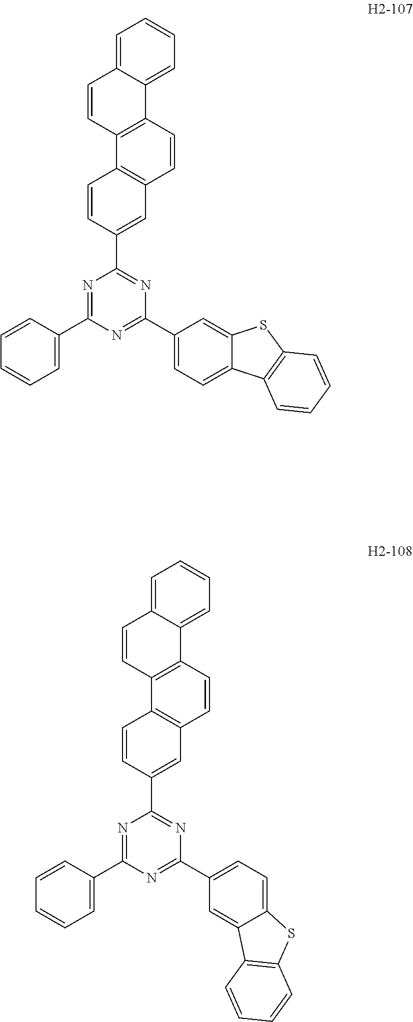 [SMILES: c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1]

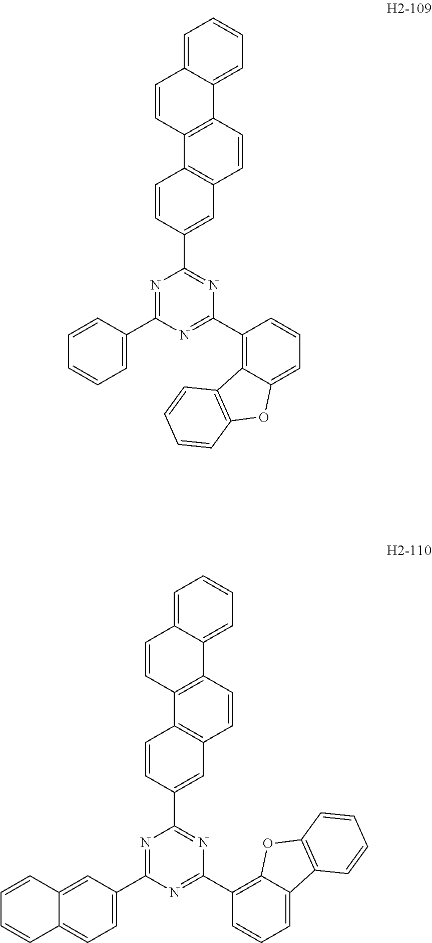 [SMILES: c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1]

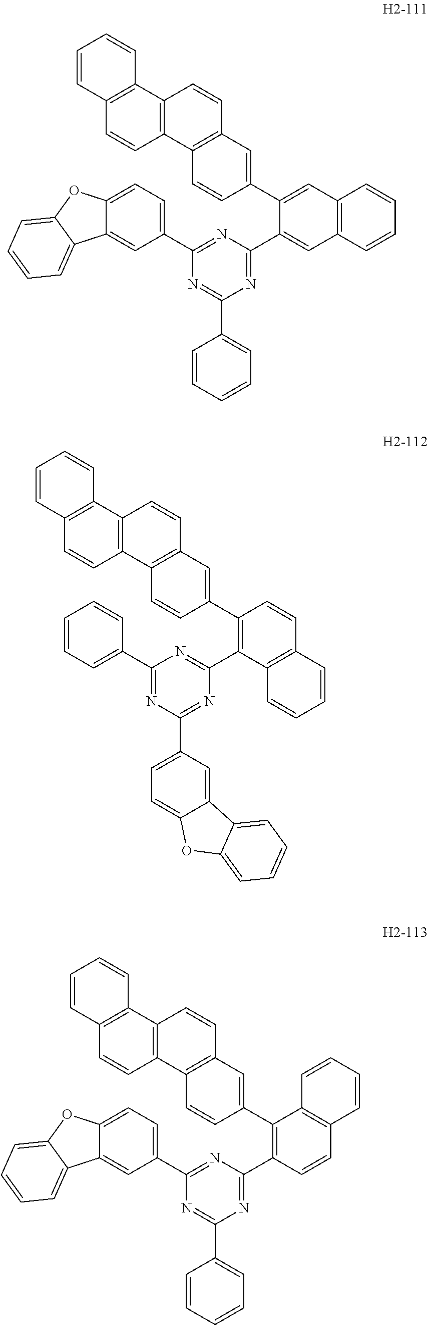 [SMILES: c1ccc(-c2nc(-c3ccc4oc5ccccc5c4c3)nc(-c3cc4ccccc4cc3-c3ccc4c(ccc5c6ccccc6ccc45)c3)n2)cc1 ; c1ccc(-c2nc(-c3ccc4oc5ccccc5c4c3)nc(-c3c(-c4ccc5c(ccc6c7ccccc7ccc56)c4)ccc4ccccc34)n2)cc1 ; c1ccc(-c2nc(-c3ccc4oc5ccccc5c4c3)nc(-c3ccc4ccccc4c3-c3ccc4c(ccc5c6ccccc6ccc45)c3)n2)cc1]

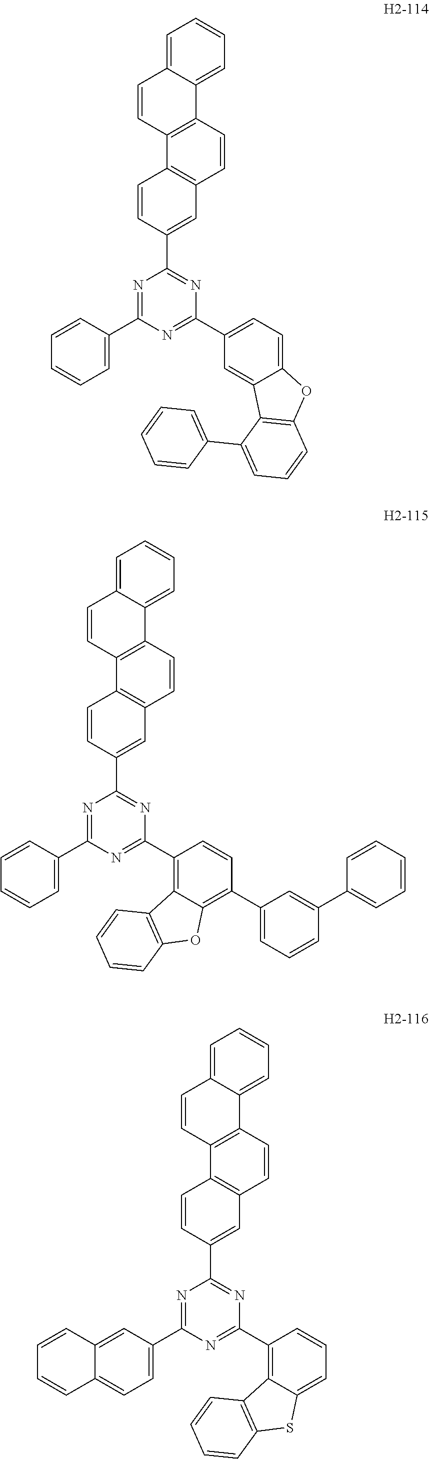 [SMILES: c1ccc(-c2nc(-c3ccc4c(ccc5c6ccccc6ccc45)c3)nc(-c3ccc4oc5cccc(-c6ccccc6)c5c4c3)n2)cc1 ; c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4cccc5sc6ccccc6c45)n3)ccc2c1]

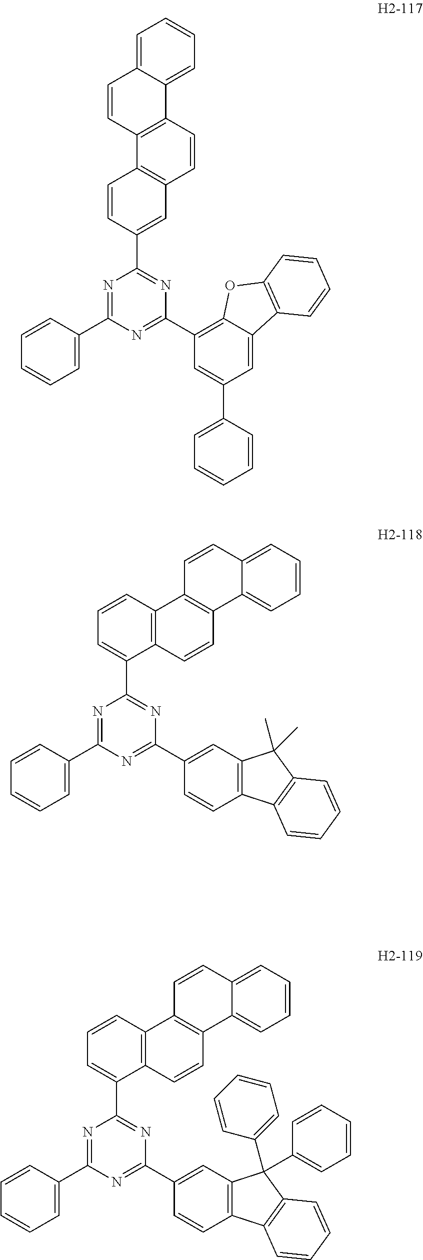 [SMILES: c1ccc(-c2cc(-c3nc(-c4ccccc4)nc(-c4ccc5c(ccc6c7ccccc7ccc56)c4)n3)c3oc4ccccc4c3c2)cc1 ; CC1(C)c2ccccc2-c2ccc(-c3nc(-c4ccccc4)nc(-c4cccc5c4ccc4c6ccccc6ccc54)n3)cc21 ; c1ccc(-c2nc(-c3ccc4c(c3)C(c3ccccc3)(c3ccccc3)c3ccccc3-4)nc(-c3cccc4c3ccc3c5ccccc5ccc43)n2)cc1]

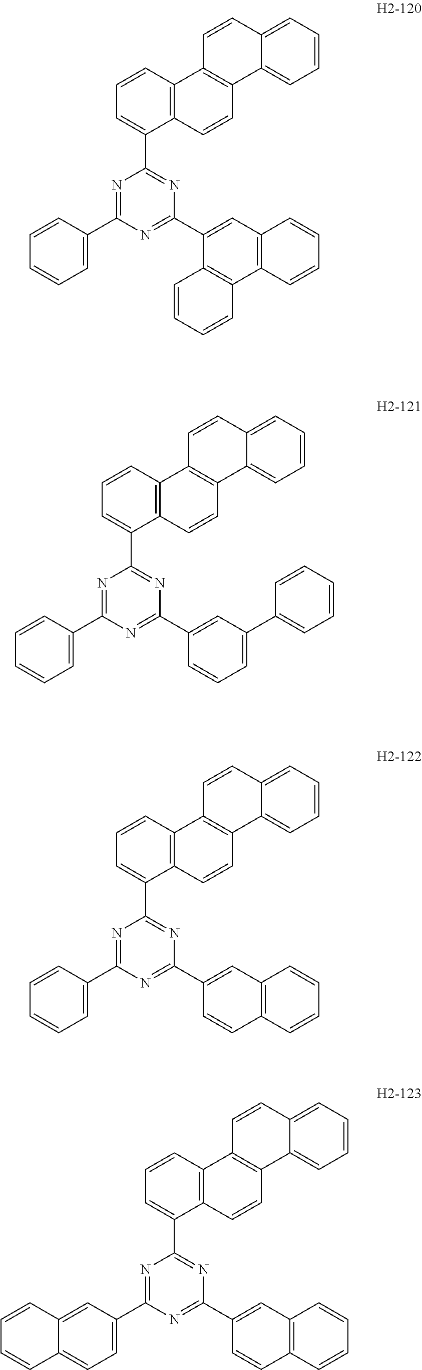 [SMILES: c1ccc(-c2nc(-c3cc4ccccc4c4ccccc34)nc(-c3cccc4c3ccc3c5ccccc5ccc43)n2)cc1 ; c1ccc(-c2cccc(-c3nc(-c4ccccc4)nc(-c4cccc5c4ccc4c6ccccc6ccc54)n3)c2)cc1 ; c1ccc(-c2nc(-c3ccc4ccccc4c3)nc(-c3cccc4c3ccc3c5ccccc5ccc43)n2)cc1 ; c1ccc2cc(-c3nc(-c4ccc5ccccc5c4)nc(-c4cccc5c4ccc4c6ccccc6ccc54)n3)ccc2c1]

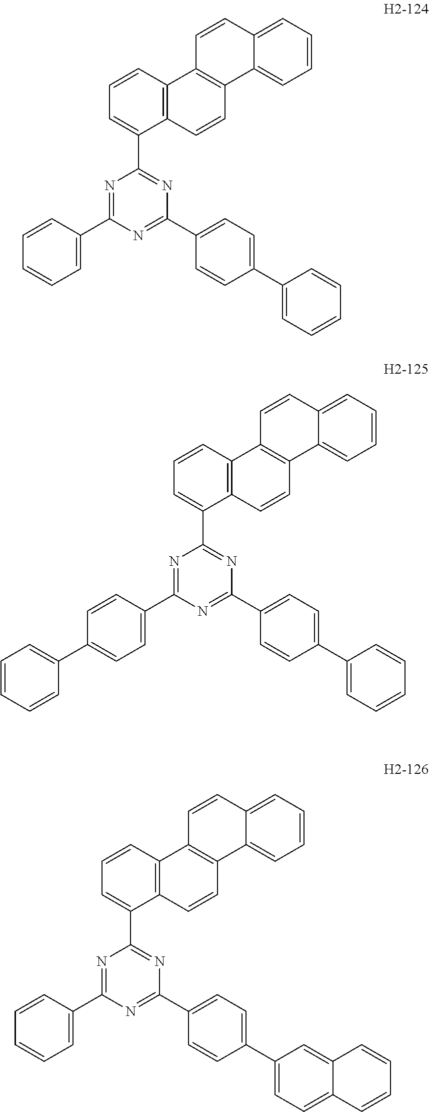 [SMILES: c1ccc(-c2ccc(-c3nc(-c4ccccc4)nc(-c4cccc5c4ccc4c6ccccc6ccc54)n3)cc2)cc1 ; c1ccc(-c2ccc(-c3nc(-c4ccc(-c5ccccc5)cc4)nc(-c4cccc5c4ccc4c6ccccc6ccc54)n3)cc2)cc1 ; c1ccc(-c2nc(-c3ccc(-c4ccc5ccccc5c4)cc3)nc(-c3cccc4c3ccc3c5ccccc5ccc43)n2)cc1]

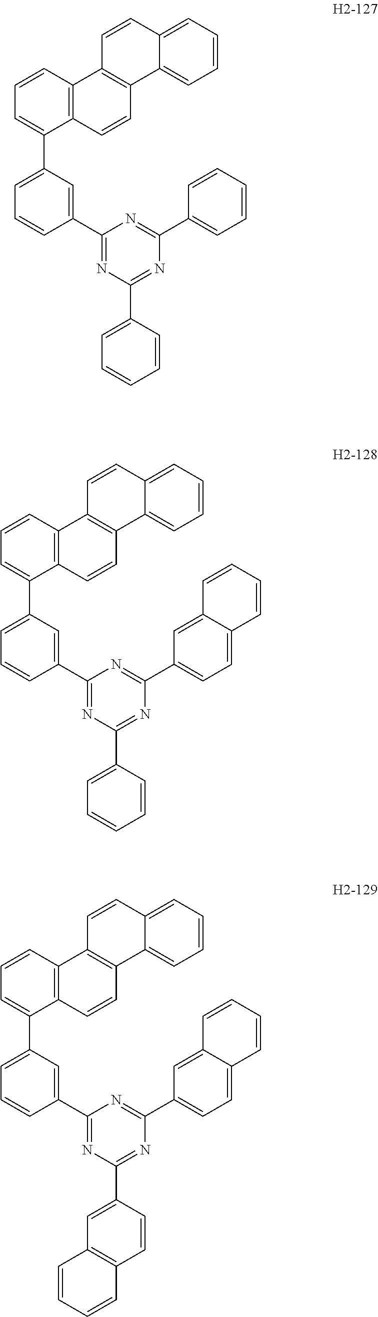 [SMILES: c1ccc(-c2nc(-c3ccccc3)nc(-c3cccc(-c4cccc5c4ccc4c6ccccc6ccc54)c3)n2)cc1 ; c1ccc(-c2nc(-c3cccc(-c4cccc5c4ccc4c6ccccc6ccc54)c3)nc(-c3ccc4ccccc4c3)n2)cc1 ; c1cc(-c2nc(-c3ccc4ccccc4c3)nc(-c3ccc4ccccc4c3)n2)cc(-c2cccc3c2ccc2c4ccccc4ccc32)c1]

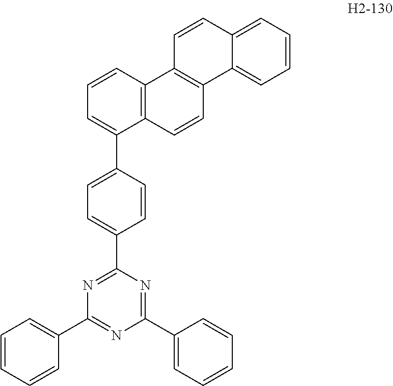 [SMILES: c1ccc(-c2nc(-c3ccccc3)nc(-c3ccc(-c4cccc5c4ccc4c6ccccc6ccc54)cc3)n2)cc1]

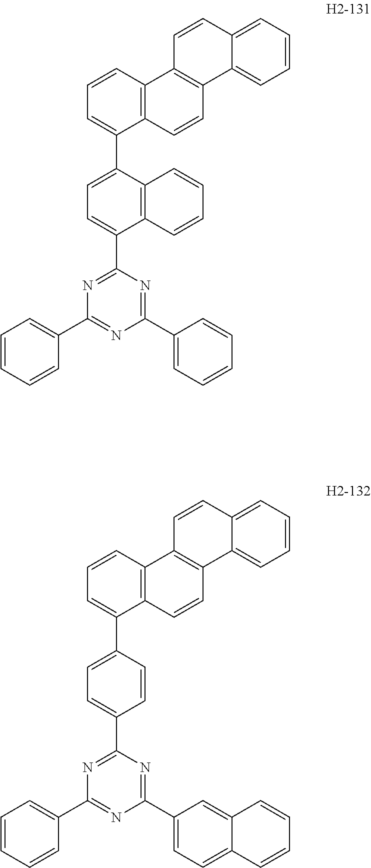 [SMILES: c1ccc(-c2nc(-c3ccccc3)nc(-c3ccc(-c4cccc5c4ccc4c6ccccc6ccc54)c4ccccc34)n2)cc1 ; c1ccc(-c2nc(-c3ccc(-c4cccc5c4ccc4c6ccccc6ccc54)cc3)nc(-c3ccc4ccccc4c3)n2)cc1]

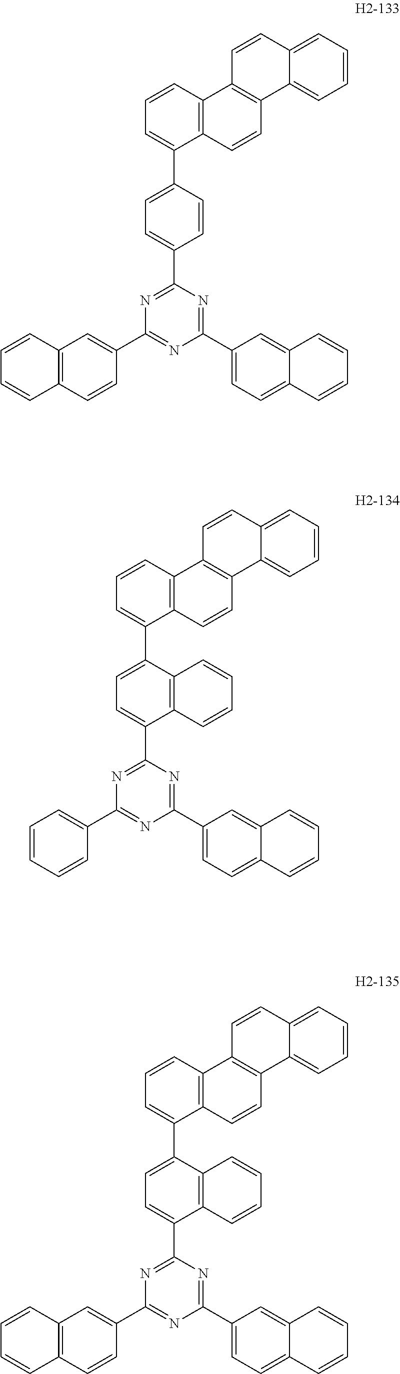 [SMILES: c1ccc2cc(-c3nc(-c4ccc(-c5cccc6c5ccc5c7ccccc7ccc65)cc4)nc(-c4ccc5ccccc5c4)n3)ccc2c1 ; c1ccc(-c2nc(-c3ccc4ccccc4c3)nc(-c3ccc(-c4cccc5c4ccc4c6ccccc6ccc54)c4ccccc34)n2)cc1 ; c1ccc2cc(-c3nc(-c4ccc5ccccc5c4)nc(-c4ccc(-c5cccc6c5ccc5c7ccccc7ccc65)c5ccccc45)n3)ccc2c1]

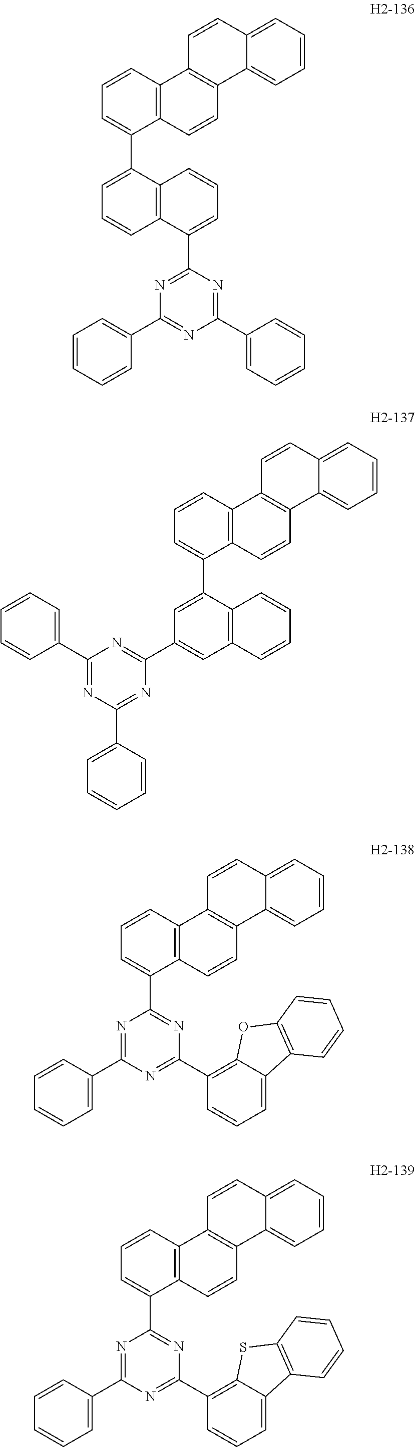 [SMILES: c1ccc(-c2nc(-c3ccccc3)nc(-c3cccc4c(-c5cccc6c5ccc5c7ccccc7ccc65)cccc34)n2)cc1 ; c1ccc(-c2nc(-c3ccccc3)nc(-c3cc(-c4cccc5c4ccc4c6ccccc6ccc54)c4ccccc4c3)n2)cc1 ; c1ccc(-c2nc(-c3cccc4c3ccc3c5ccccc5ccc43)nc(-c3cccc4c3oc3ccccc34)n2)cc1 ; c1ccc(-c2nc(-c3cccc4c3ccc3c5ccccc5ccc43)nc(-c3cccc4c3sc3ccccc34)n2)cc1]

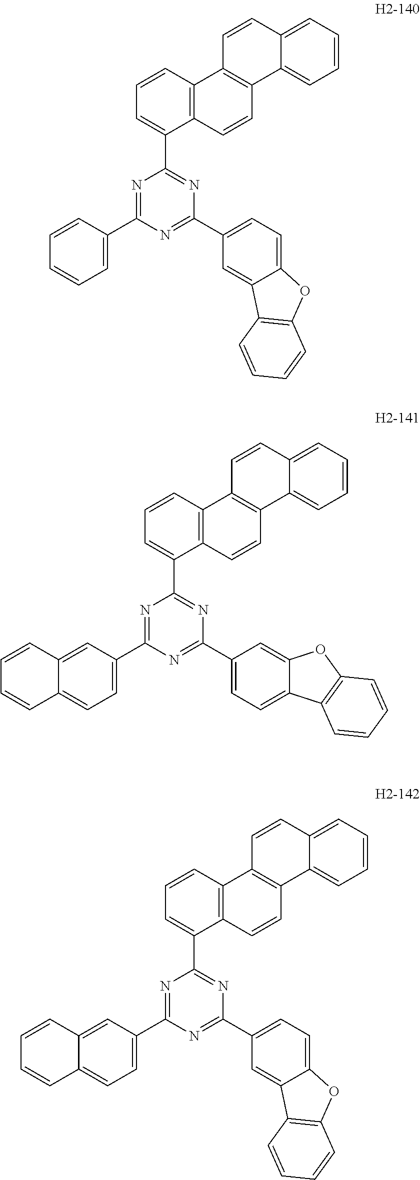 [SMILES: c1ccc(-c2nc(-c3ccc4oc5ccccc5c4c3)nc(-c3cccc4c3ccc3c5ccccc5ccc43)n2)cc1 ; c1ccc2cc(-c3nc(-c4ccc5c(c4)oc4ccccc45)nc(-c4cccc5c4ccc4c6ccccc6ccc54)n3)ccc2c1 ; c1ccc2cc(-c3nc(-c4ccc5oc6ccccc6c5c4)nc(-c4cccc5c4ccc4c6ccccc6ccc54)n3)ccc2c1]

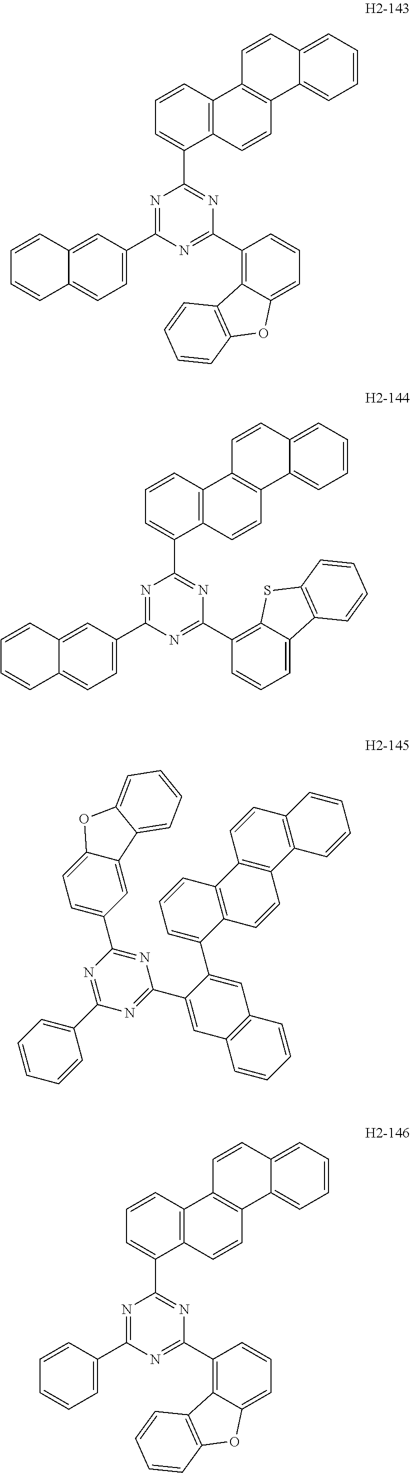 [SMILES: c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4oc5ccccc5c34)n2)cc1]

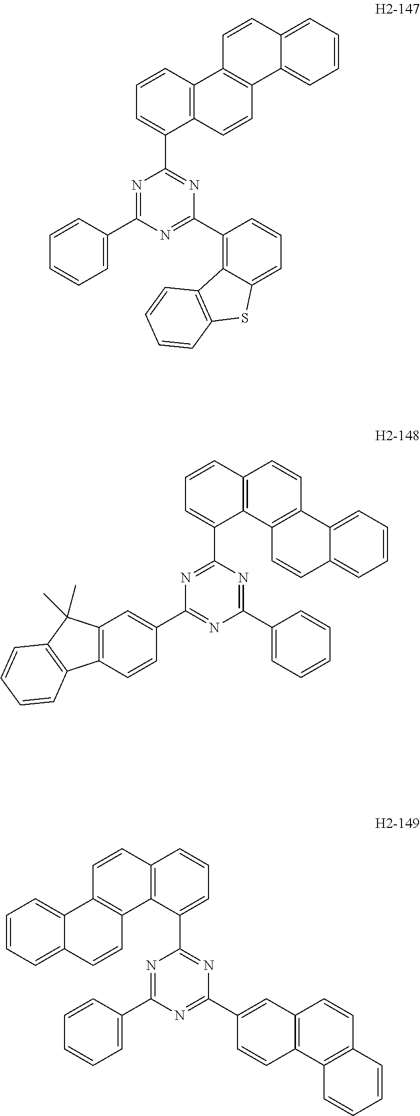 [SMILES: c1ccc(-c2nc(-c3cccc4c3ccc3c5ccccc5ccc43)nc(-c3cccc4sc5ccccc5c34)n2)cc1 ; CC1(C)c2ccccc2-c2ccc(-c3nc(-c4ccccc4)nc(-c4cccc5ccc6c7ccccc7ccc6c45)n3)cc21 ; c1ccc(-c2nc(-c3ccc4c(ccc5ccccc54)c3)nc(-c3cccc4ccc5c6ccccc6ccc5c34)n2)cc1]

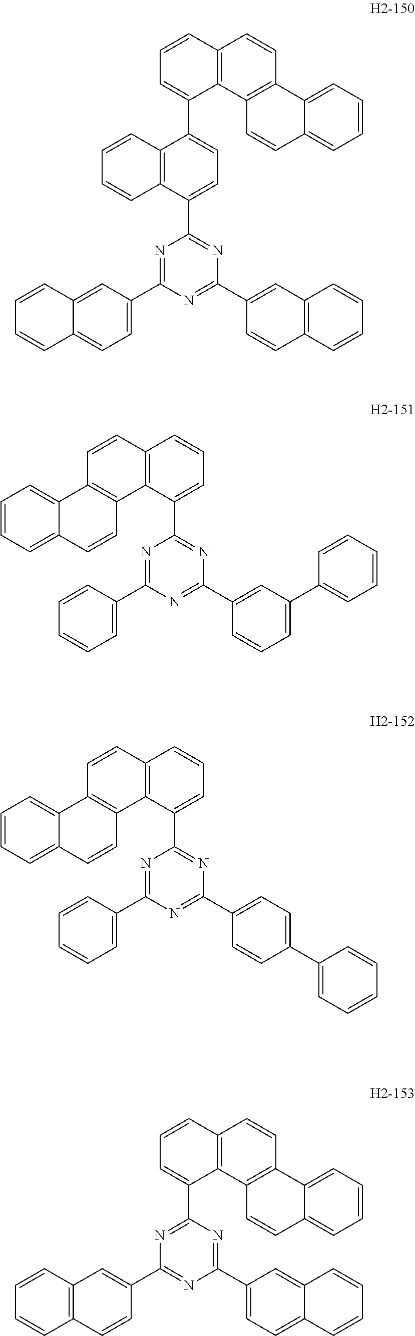 [SMILES: c1ccc2cc(-c3nc(-c4ccc5ccccc5c4)nc(-c4ccc(-c5cccc6ccc7c8ccccc8ccc7c56)c5ccccc45)n3)ccc2c1 ; c1ccc(-c2cccc(-c3nc(-c4ccccc4)nc(-c4cccc5ccc6c7ccccc7ccc6c45)n3)c2)cc1 ; c1ccc(-c2ccc(-c3nc(-c4ccccc4)nc(-c4cccc5ccc6c7ccccc7ccc6c45)n3)cc2)cc1 ; c1ccc2cc(-c3nc(-c4ccc5ccccc5c4)nc(-c4cccc5ccc6c7ccccc7ccc6c45)n3)ccc2c1]

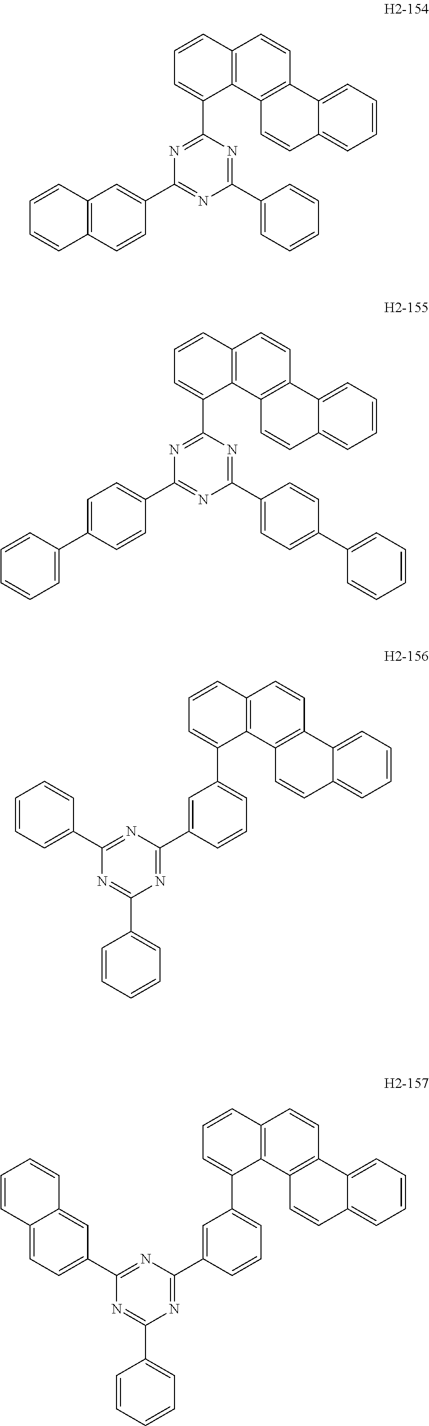 [SMILES: c1ccc(-c2nc(-c3ccc4ccccc4c3)nc(-c3cccc4ccc5c6ccccc6ccc5c34)n2)cc1 ; c1ccc(-c2ccc(-c3nc(-c4ccc(-c5ccccc5)cc4)nc(-c4cccc5ccc6c7ccccc7ccc6c45)n3)cc2)cc1 ; c1ccc(-c2nc(-c3ccccc3)nc(-c3cccc(-c4cccc5ccc6c7ccccc7ccc6c45)c3)n2)cc1 ; c1ccc(-c2nc(-c3cccc(-c4cccc5ccc6c7ccccc7ccc6c45)c3)nc(-c3ccc4ccccc4c3)n2)cc1]

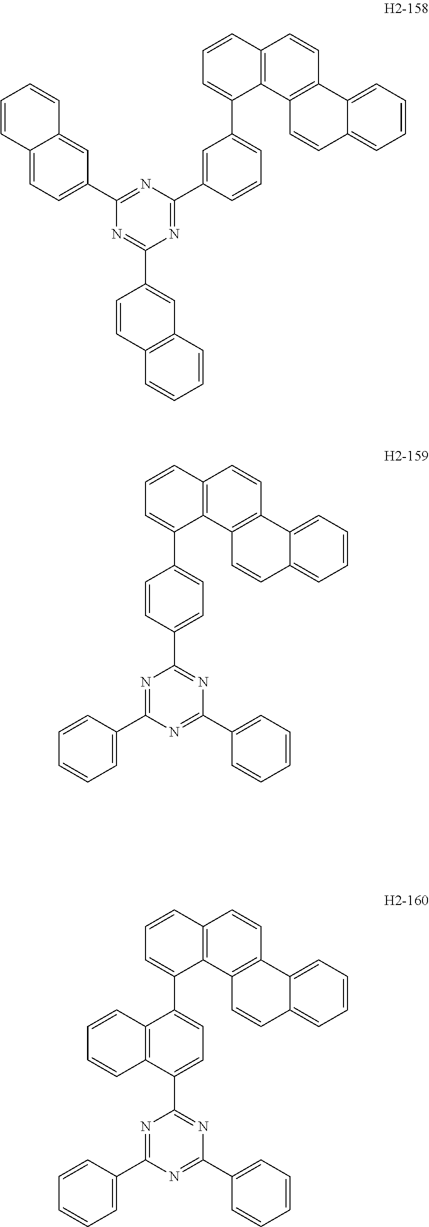 [SMILES: c1cc(-c2nc(-c3ccc4ccccc4c3)nc(-c3ccc4ccccc4c3)n2)cc(-c2cccc3ccc4c5ccccc5ccc4c23)c1 ; c1ccc(-c2nc(-c3ccccc3)nc(-c3ccc(-c4cccc5ccc6c7ccccc7ccc6c45)cc3)n2)cc1 ; c1ccc(-c2nc(-c3ccccc3)nc(-c3ccc(-c4cccc5ccc6c7ccccc7ccc6c45)c4ccccc34)n2)cc1]

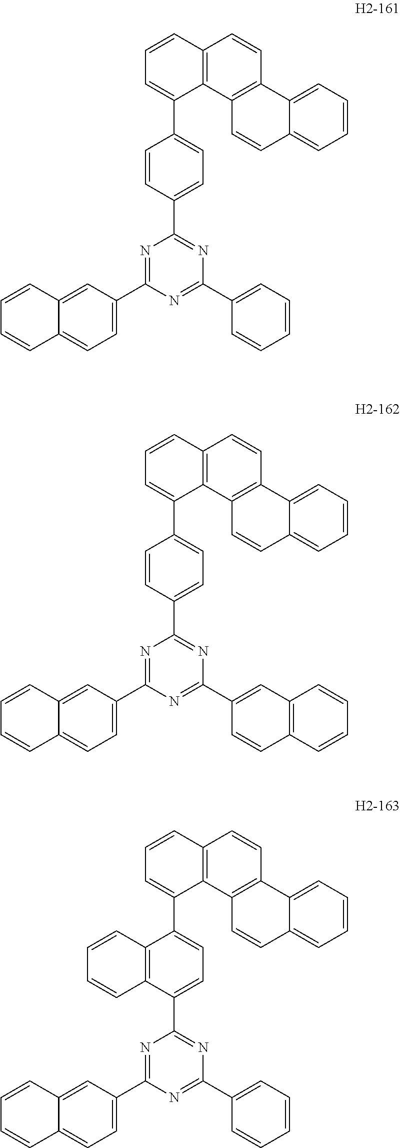 [SMILES: c1ccc(-c2nc(-c3ccc(-c4cccc5ccc6c7ccccc7ccc6c45)cc3)nc(-c3ccc4ccccc4c3)n2)cc1 ; c1ccc2cc(-c3nc(-c4ccc(-c5cccc6ccc7c8ccccc8ccc7c56)cc4)nc(-c4ccc5ccccc5c4)n3)ccc2c1 ; c1ccc(-c2nc(-c3ccc4ccccc4c3)nc(-c3ccc(-c4cccc5ccc6c7ccccc7ccc6c45)c4ccccc34)n2)cc1]

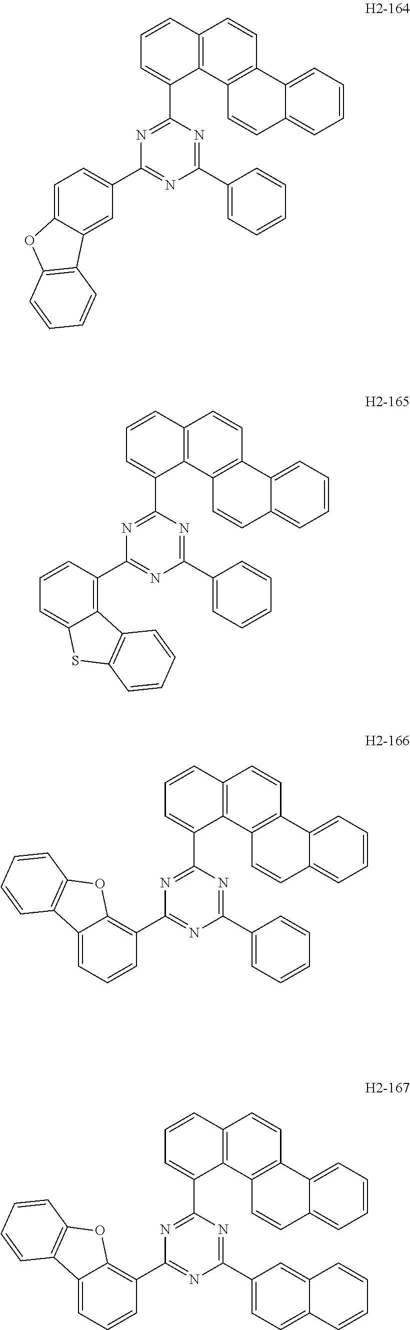 [SMILES: c1ccc(-c2nc(-c3ccc4oc5ccccc5c4c3)nc(-c3cccc4ccc5c6ccccc6ccc5c34)n2)cc1 ; c1ccc(-c2nc(-c3cccc4ccc5c6ccccc6ccc5c34)nc(-c3cccc4sc5ccccc5c34)n2)cc1 ; c1ccc(-c2nc(-c3cccc4c3oc3ccccc34)nc(-c3cccc4ccc5c6ccccc6ccc5c34)n2)cc1 ; c1ccc2cc(-c3nc(-c4cccc5c4oc4ccccc45)nc(-c4cccc5ccc6c7ccccc7ccc6c45)n3)ccc2c1]

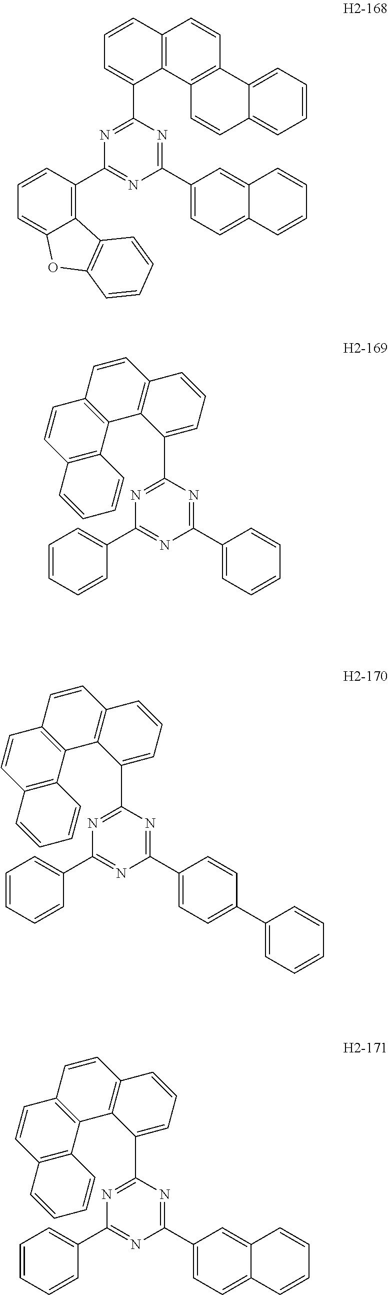 [SMILES: c1ccc2cc(-c3nc(-c4cccc5ccc6c7ccccc7ccc6c45)nc(-c4cccc5oc6ccccc6c45)n3)ccc2c1 ; c1ccc(-c2nc(-c3ccccc3)nc(-c3cccc4ccc5ccc6ccccc6c5c34)n2)cc1 ; c1ccc(-c2ccc(-c3nc(-c4ccccc4)nc(-c4cccc5ccc6ccc7ccccc7c6c45)n3)cc2)cc1 ; c1ccc(-c2nc(-c3ccc4ccccc4c3)nc(-c3cccc4ccc5ccc6ccccc6c5c34)n2)cc1]

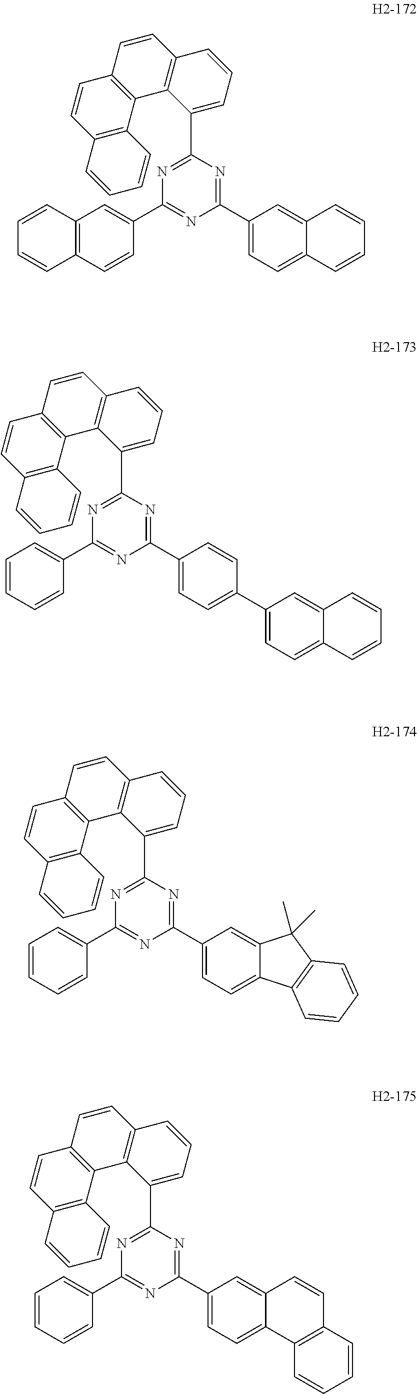 [SMILES: c1ccc2cc(-c3nc(-c4ccc5ccccc5c4)nc(-c4cccc5ccc6ccc7ccccc7c6c45)n3)ccc2c1 ; c1ccc(-c2nc(-c3ccc(-c4ccc5ccccc5c4)cc3)nc(-c3cccc4ccc5ccc6ccccc6c5c34)n2)cc1 ; CC1(C)c2ccccc2-c2ccc(-c3nc(-c4ccccc4)nc(-c4cccc5ccc6ccc7ccccc7c6c45)n3)cc21 ; c1ccc(-c2nc(-c3ccc4c(ccc5ccccc54)c3)nc(-c3cccc4ccc5ccc6ccccc6c5c34)n2)cc1]

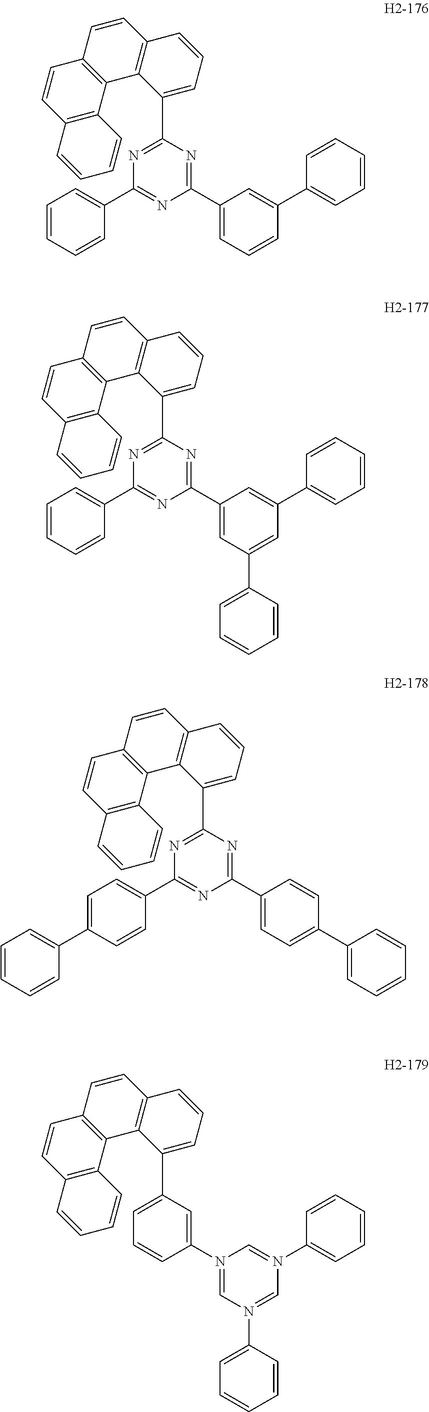

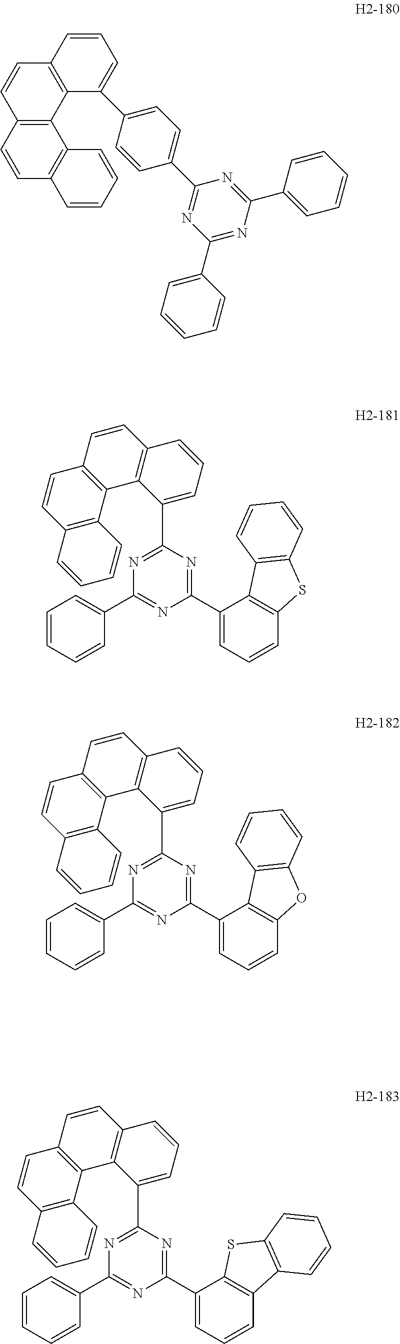 [SMILES: c1ccc(-c2nc(-c3ccccc3)nc(-c3ccc(-c4cccc5ccc6ccc7ccccc7c6c45)cc3)n2)cc1 ; c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1]

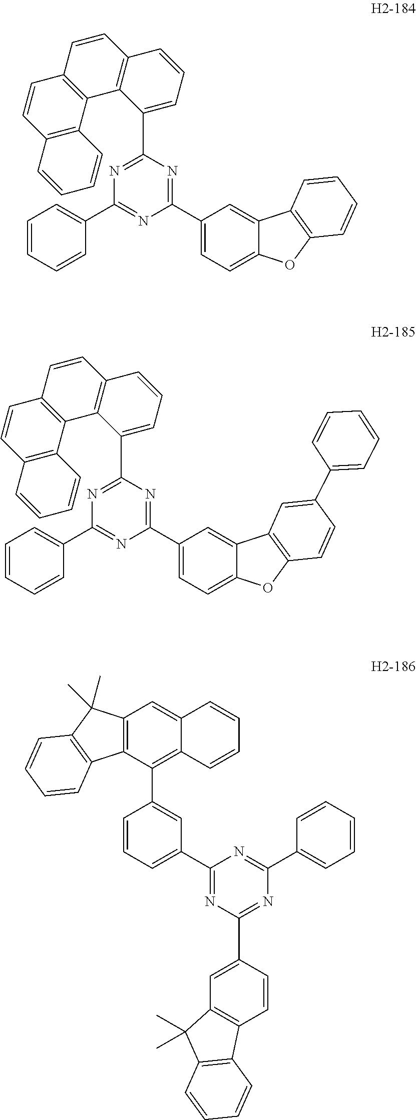 [SMILES: c1ccc(-c2nc(-c3ccc4oc5ccccc5c4c3)nc(-c3cccc4ccc5ccc6ccccc6c5c34)n2)cc1 ; c1ccc(-c2ccc3oc4ccc(-c5nc(-c6ccccc6)nc(-c6cccc7ccc8ccc9ccccc9c8c67)n5)cc4c3c2)cc1 ; CC1(C)c2ccccc2-c2ccc(-c3nc(-c4ccccc4)nc(-c4cccc(-c5c6c(cc7ccccc57)C(C)(C)c5ccccc5-6)c4)n3)cc21]

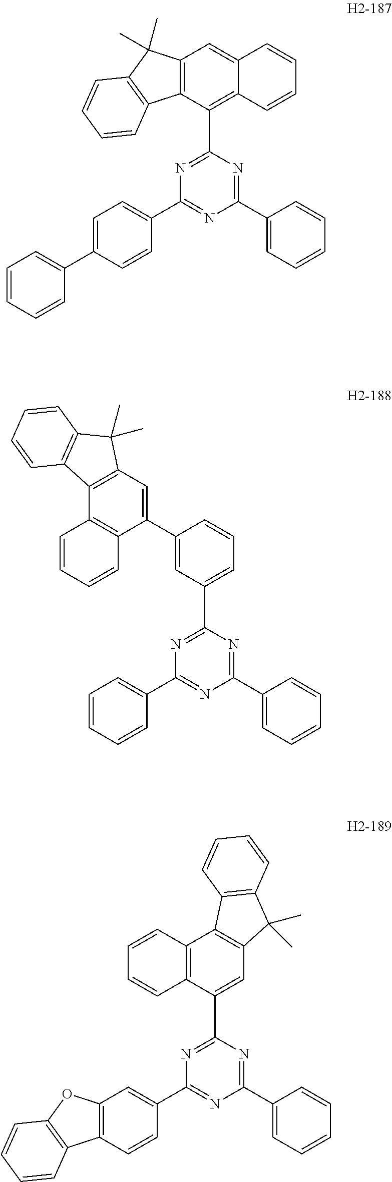 [SMILES: CC1(C)c2ccccc2-c2c1cc1ccccc1c2-c1nc(-c2ccccc2)nc(-c2ccc(-c3ccccc3)cc2)n1 ; CC1(C)c2ccccc2-c2c1cc(-c1cccc(-c3nc(-c4ccccc4)nc(-c4ccccc4)n3)c1)c1ccccc21 ; CC1(C)c2ccccc2-c2c1cc(-c1nc(-c3ccccc3)nc(-c3ccc4c(c3)oc3ccccc34)n1)c1ccccc21]

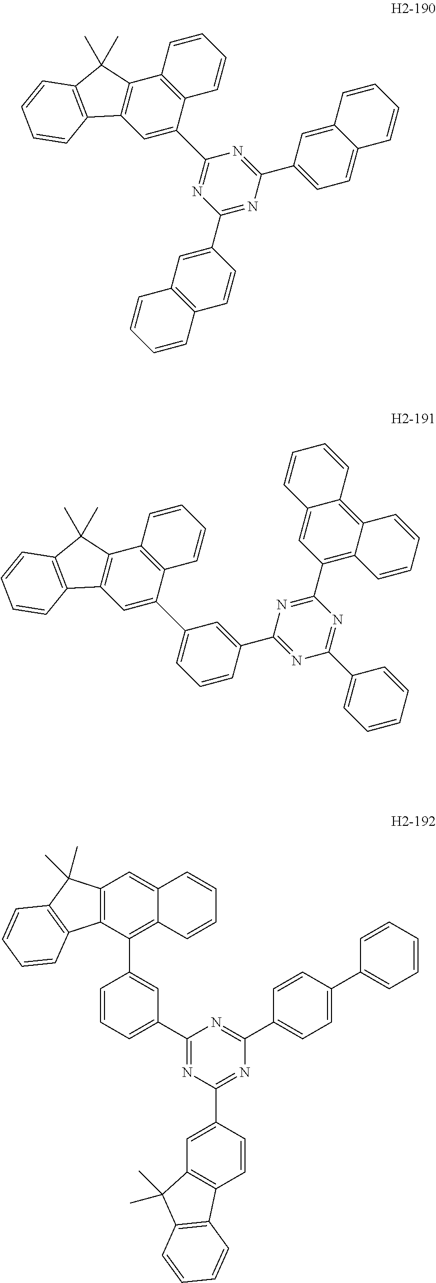 [SMILES: CC1(C)c2ccccc2-c2cc(-c3nc(-c4ccc5ccccc5c4)nc(-c4ccc5ccccc5c4)n3)c3ccccc3c21 ; CC1(C)c2ccccc2-c2cc(-c3cccc(-c4nc(-c5ccccc5)nc(-c5cc6ccccc6c6ccccc56)n4)c3)c3ccccc3c21 ; CC1(C)c2ccccc2-c2ccc(-c3nc(-c4ccc(-c5ccccc5)cc4)nc(-c4cccc(-c5c6c(cc7ccccc57)C(C)(C)c5ccccc5-6)c4)n3)cc21]

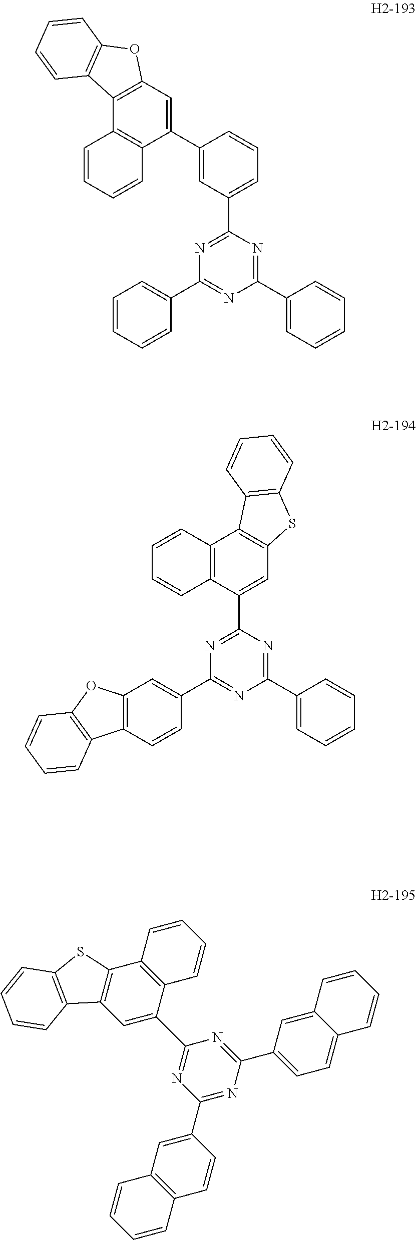 [SMILES: c1ccc(-c2nc(-c3ccccc3)nc(-c3cccc(-c4cc5oc6ccccc6c5c5ccccc45)c3)n2)cc1 ; c1ccc(-c2nc(-c3ccc4c(c3)oc3ccccc34)nc(-c3cc4sc5ccccc5c4c4ccccc34)n2)cc1 ; c1ccc2cc(-c3nc(-c4ccc5ccccc5c4)nc(-c4cc5c6ccccc6sc5c5ccccc45)n3)ccc2c1]

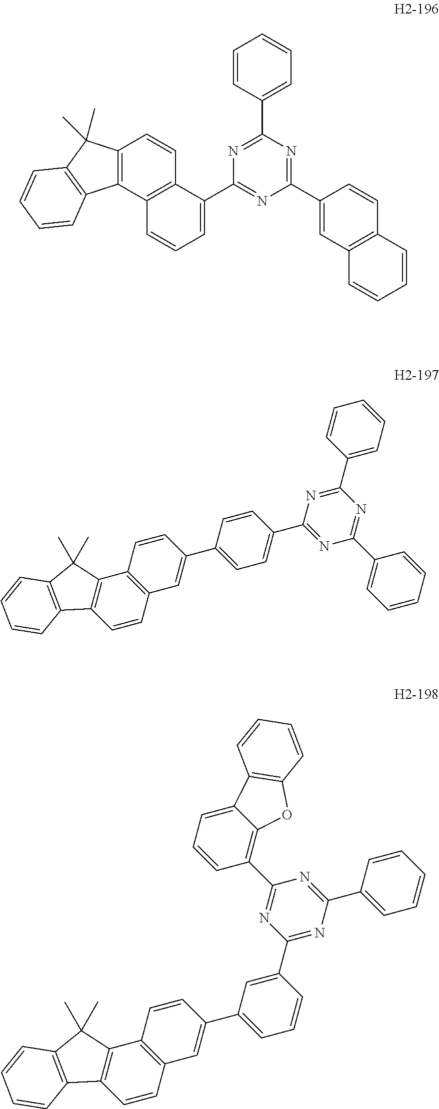 [SMILES: CC1(C)c2ccccc2-c2c1ccc1c(-c3nc(-c4ccccc4)nc(-c4ccc5ccccc5c4)n3)cccc21 ; CC1(C)c2ccccc2-c2ccc3cc(-c4ccc(-c5nc(-c6ccccc6)nc(-c6ccccc6)n5)cc4)ccc3c21 ; CC1(C)c2ccccc2-c2ccc3cc(-c4cccc(-c5nc(-c6ccccc6)nc(-c6cccc7c6oc6ccccc67)n5)c4)ccc3c21]

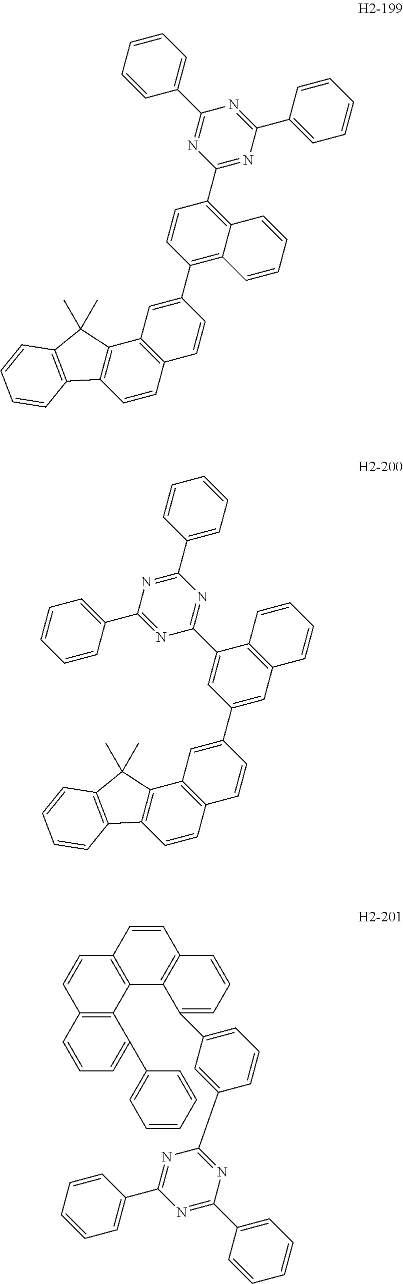 [SMILES: CC1(C)c2ccccc2-c2ccc3ccc(-c4ccc(-c5nc(-c6ccccc6)nc(-c6ccccc6)n5)c5ccccc45)cc3c21 ; CC1(C)c2ccccc2-c2ccc3ccc(-c4cc(-c5nc(-c6ccccc6)nc(-c6ccccc6)n5)c5ccccc5c4)cc3c21 ; c1ccc(-c2nc(-c3ccccc3)nc(-c3cccc(-c4cccc5ccc6ccc7cccc(-c8ccccc8)c7c6c45)c3)n2)cc1]

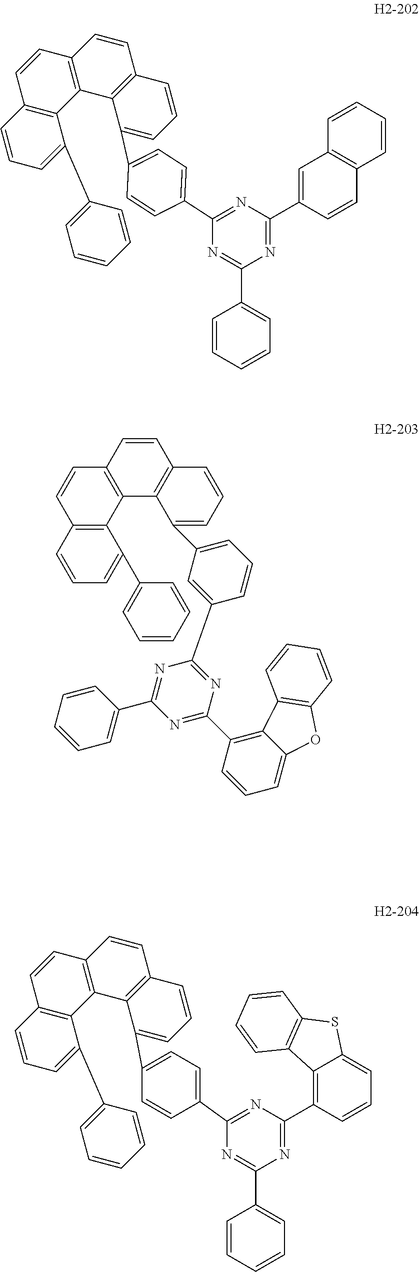 [SMILES: c1ccc(-c2nc(-c3ccc(-c4cccc5ccc6ccc7cccc(-c8ccccc8)c7c6c45)cc3)nc(-c3ccc4ccccc4c3)n2)cc1 ; c1ccc(-c2nc(-c3cccc(-c4cccc5ccc6ccc7cccc(-c8ccccc8)c7c6c45)c3)nc(-c3cccc4oc5ccccc5c34)n2)cc1 ; c1ccc(-c2nc(-c3ccc(-c4cccc5ccc6ccc7cccc(-c8ccccc8)c7c6c45)cc3)nc(-c3cccc4sc5ccccc5c34)n2)cc1]

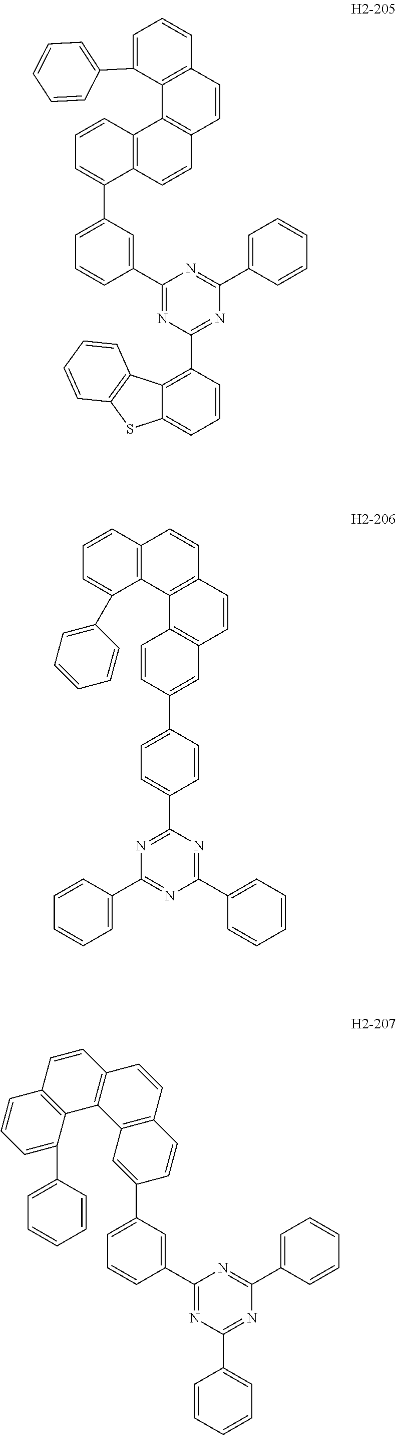 [SMILES: c1ccc(-c2nc(-c3cccc(-c4cccc5c4ccc4ccc6cccc(-c7ccccc7)c6c45)c3)nc(-c3cccc4sc5ccccc5c34)n2)cc1 ; c1ccc(-c2nc(-c3ccccc3)nc(-c3ccc(-c4ccc5c(ccc6ccc7cccc(-c8ccccc8)c7c65)c4)cc3)n2)cc1 ; c1ccc(-c2nc(-c3ccccc3)nc(-c3cccc(-c4ccc5ccc6ccc7cccc(-c8ccccc8)c7c6c5c4)c3)n2)cc1]

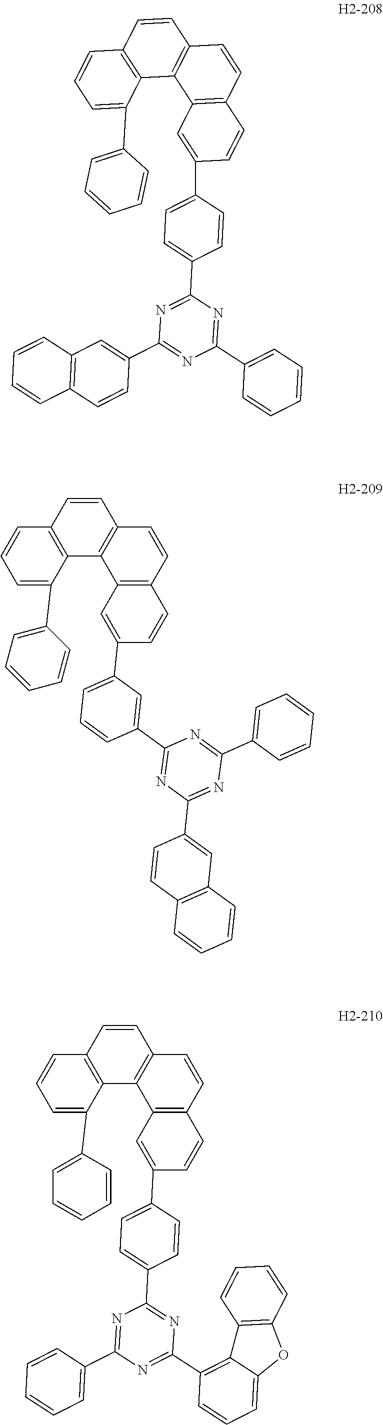 [SMILES: c1ccc(-c2nc(-c3ccc(-c4ccc5ccc6ccc7cccc(-c8ccccc8)c7c6c5c4)cc3)nc(-c3ccc4ccccc4c3)n2)cc1 ; c1ccc(-c2nc(-c3cccc(-c4ccc5ccc6ccc7cccc(-c8ccccc8)c7c6c5c4)c3)nc(-c3ccc4ccccc4c3)n2)cc1 ; c1ccc(-c2nc(-c3ccc(-c4ccc5ccc6ccc7cccc(-c8ccccc8)c7c6c5c4)cc3)nc(-c3cccc4oc5ccccc5c34)n2)cc1]

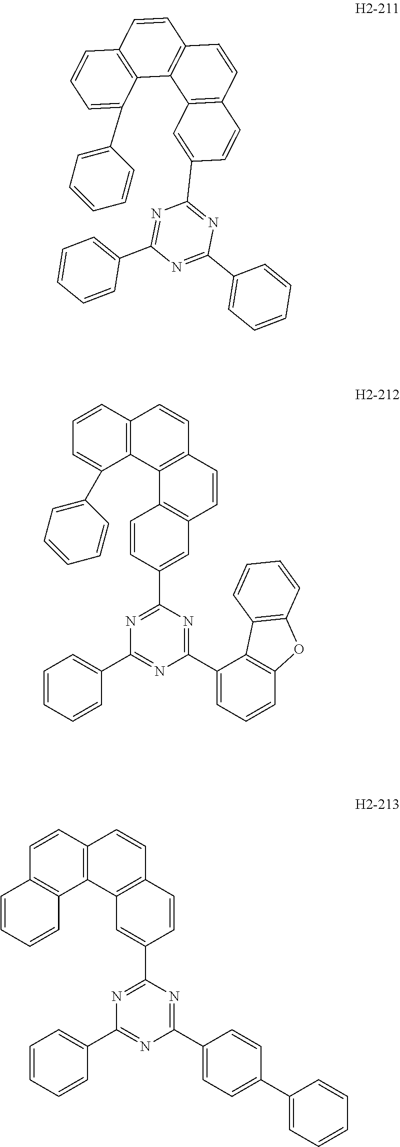 [SMILES: c1ccc(-c2nc(-c3ccccc3)nc(-c3ccc4ccc5ccc6cccc(-c7ccccc7)c6c5c4c3)n2)cc1 ; c1ccc(-c2nc(-c3ccc4c(ccc5ccc6cccc(-c7ccccc7)c6c54)c3)nc(-c3cccc4oc5ccccc5c34)n2)cc1 ; c1ccc(-c2ccc(-c3nc(-c4ccccc4)nc(-c4ccc5ccc6ccc7ccccc7c6c5c4)n3)cc2)cc1]

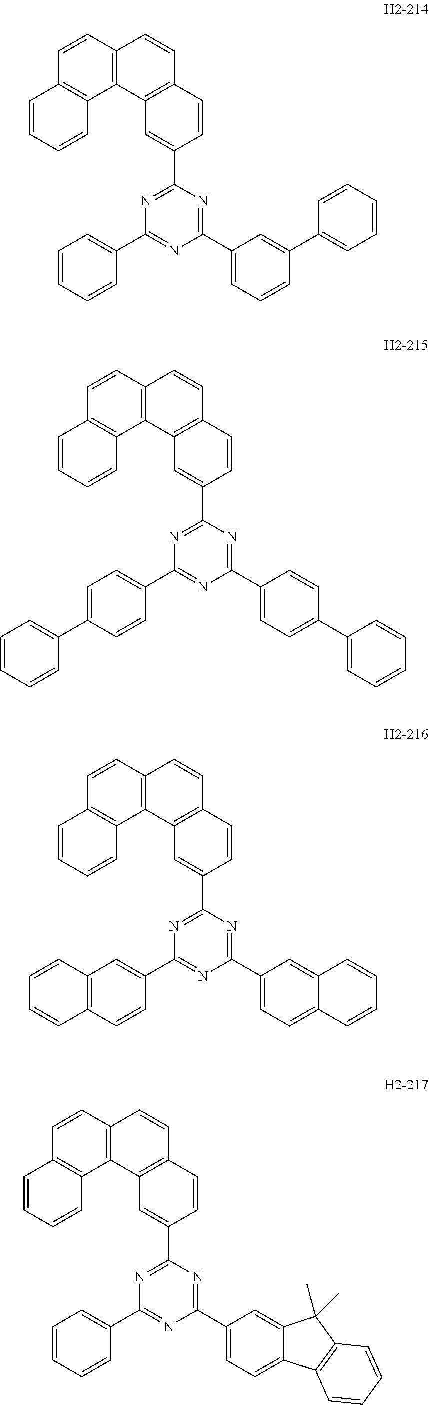 [SMILES: c1ccc(-c2cccc(-c3nc(-c4ccccc4)nc(-c4ccc5ccc6ccc7ccccc7c6c5c4)n3)c2)cc1 ; c1ccc(-c2ccc(-c3nc(-c4ccc(-c5ccccc5)cc4)nc(-c4ccc5ccc6ccc7ccccc7c6c5c4)n3)cc2)cc1 ; c1ccc2cc(-c3nc(-c4ccc5ccccc5c4)nc(-c4ccc5ccc6ccc7ccccc7c6c5c4)n3)ccc2c1 ; CC1(C)c2ccccc2-c2ccc(-c3nc(-c4ccccc4)nc(-c4ccc5ccc6ccc7ccccc7c6c5c4)n3)cc21]

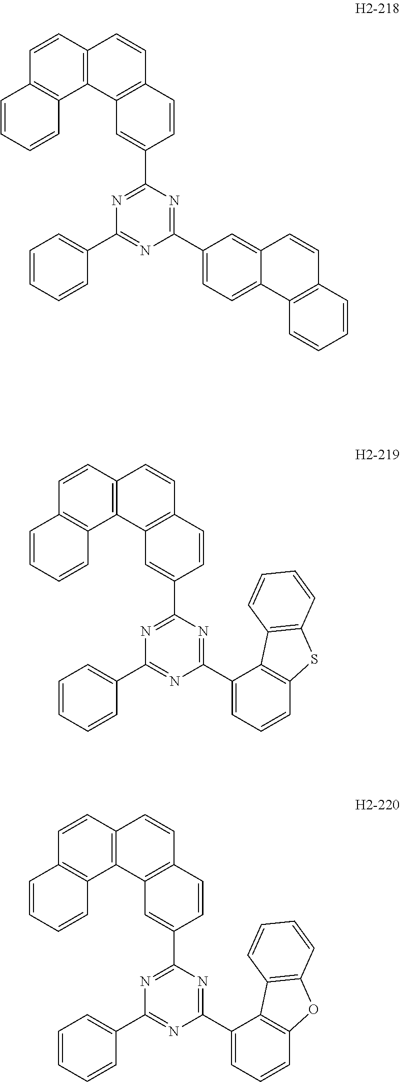 [SMILES: c1ccc(-c2nc(-c3ccc4c(ccc5ccccc54)c3)nc(-c3ccc4ccc5ccc6ccccc6c5c4c3)n2)cc1 ; c1ccc(-c2nc(-c3ccc4ccc5ccc6ccccc6c5c4c3)nc(-c3cccc4sc5ccccc5c34)n2)cc1 ; c1ccc(-c2nc(-c3ccc4ccc5ccc6ccccc6c5c4c3)nc(-c3cccc4oc5ccccc5c34)n2)cc1]

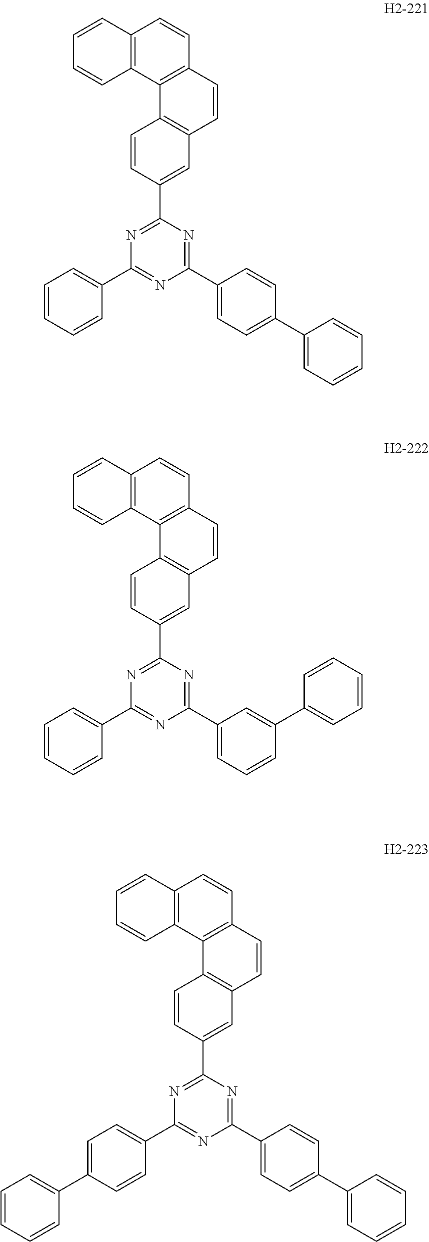 [SMILES: c1ccc(-c2ccc(-c3nc(-c4ccccc4)nc(-c4ccc5c(ccc6ccc7ccccc7c65)c4)n3)cc2)cc1 ; c1ccc(-c2cccc(-c3nc(-c4ccccc4)nc(-c4ccc5c(ccc6ccc7ccccc7c65)c4)n3)c2)cc1 ; c1ccc(-c2ccc(-c3nc(-c4ccc(-c5ccccc5)cc4)nc(-c4ccc5c(ccc6ccc7ccccc7c65)c4)n3)cc2)cc1]

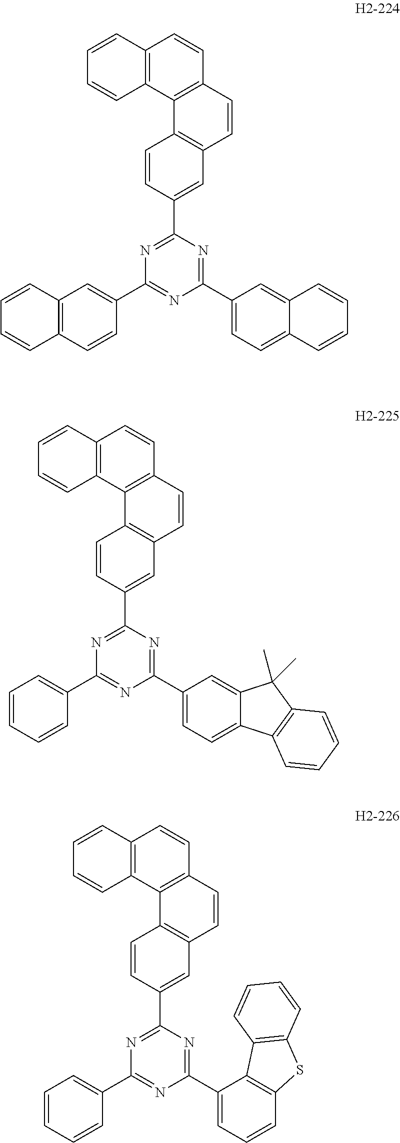 [SMILES: c1ccc2cc(-c3nc(-c4ccc5ccccc5c4)nc(-c4ccc5c(ccc6ccc7ccccc7c65)c4)n3)ccc2c1 ; CC1(C)c2ccccc2-c2ccc(-c3nc(-c4ccccc4)nc(-c4ccc5c(ccc6ccc7ccccc7c65)c4)n3)cc21 ; c1ccc(-c2nc(-c3ccc4c(ccc5ccc6ccccc6c54)c3)nc(-c3cccc4sc5ccccc5c34)n2)cc1]

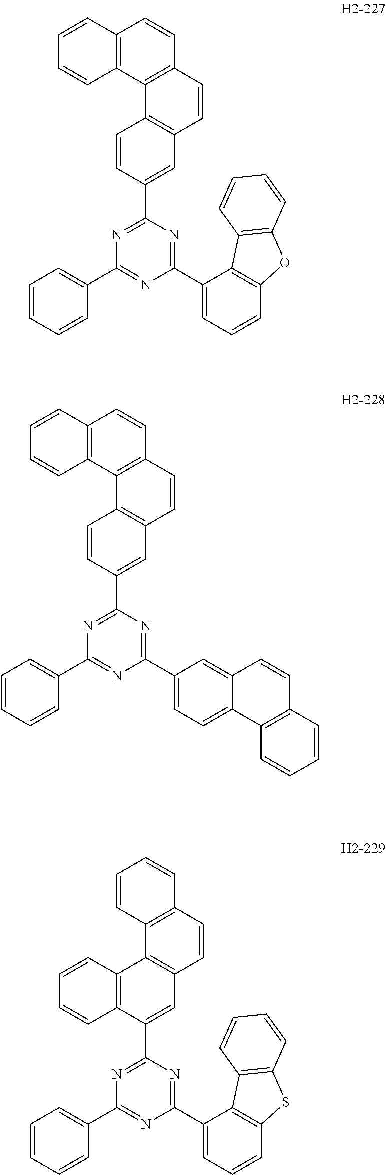 [SMILES: c1ccc(-c2nc(-c3ccc4c(ccc5ccc6ccccc6c54)c3)nc(-c3cccc4oc5ccccc5c34)n2)cc1 ; c1ccc(-c2nc(-c3ccc4c(ccc5ccccc54)c3)nc(-c3ccc4c(ccc5ccc6ccccc6c54)c3)n2)cc1 ; c1ccc(-c2nc(-c3cc4ccc5ccccc5c4c4ccccc34)nc(-c3cccc4sc5ccccc5c34)n2)cc1]

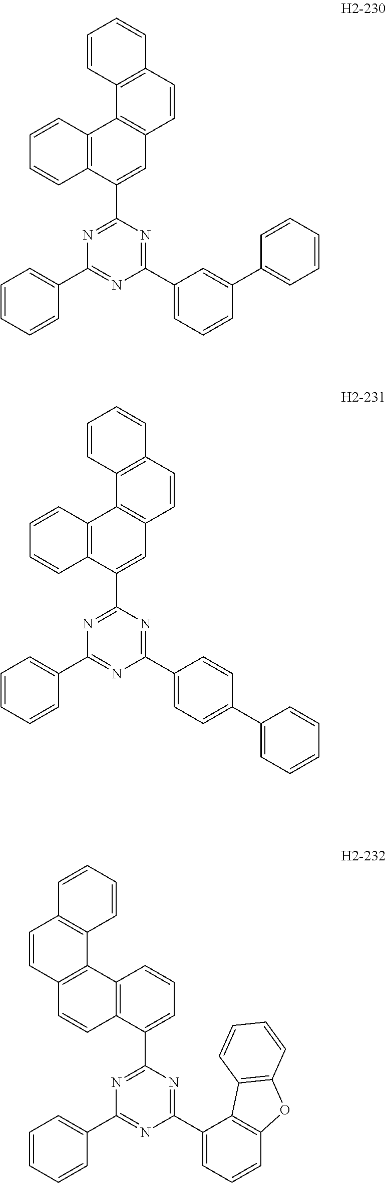 [SMILES: c1ccc(-c2cccc(-c3nc(-c4ccccc4)nc(-c4cc5ccc6ccccc6c5c5ccccc45)n3)c2)cc1 ; c1ccc(-c2ccc(-c3nc(-c4ccccc4)nc(-c4cc5ccc6ccccc6c5c5ccccc45)n3)cc2)cc1 ; c1ccc(-c2nc(-c3cccc4c3ccc3ccc5ccccc5c34)nc(-c3cccc4oc5ccccc5c34)n2)cc1]

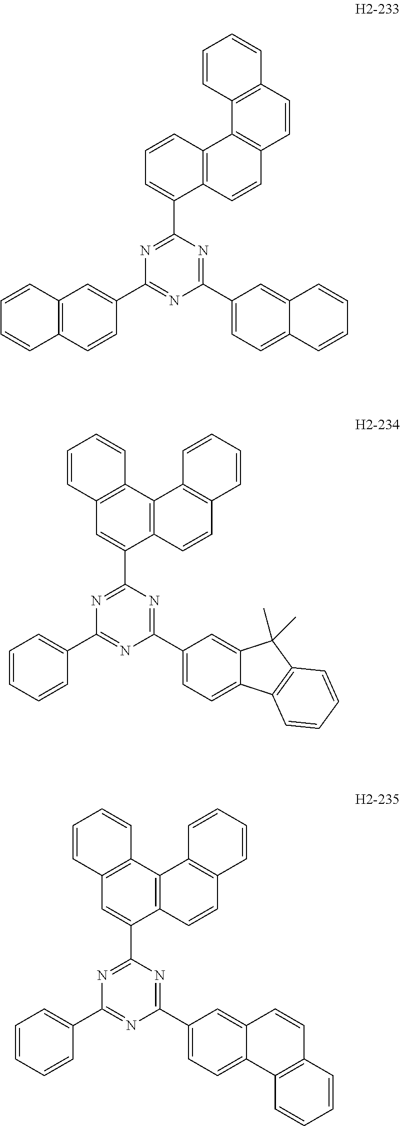 [SMILES: c1ccc2cc(-c3nc(-c4ccc5ccccc5c4)nc(-c4cccc5c4ccc4ccc6ccccc6c45)n3)ccc2c1 ; CC1(C)c2ccccc2-c2ccc(-c3nc(-c4ccccc4)nc(-c4cc5ccccc5c5c4ccc4ccccc45)n3)cc21 ; c1ccc(-c2nc(-c3ccc4c(ccc5ccccc54)c3)nc(-c3cc4ccccc4c4c3ccc3ccccc34)n2)cc1]

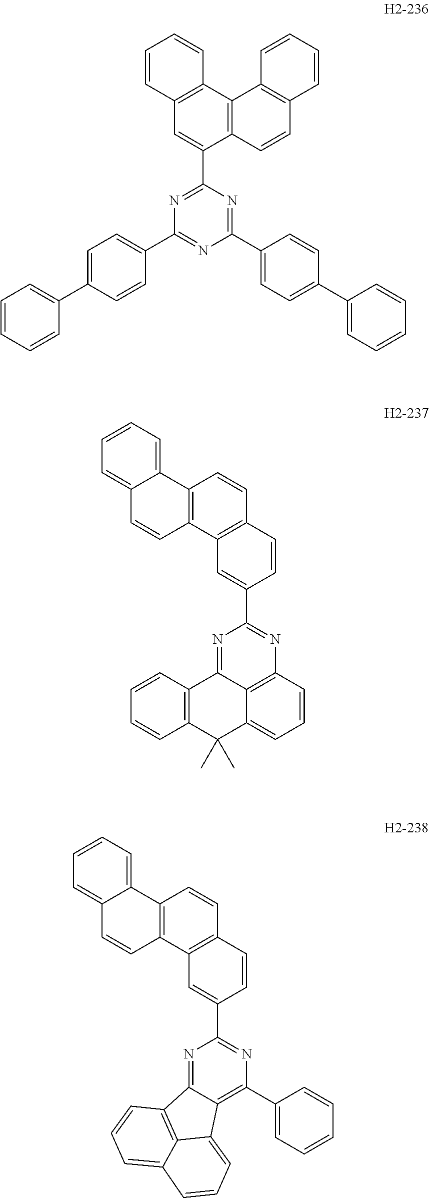 [SMILES: c1ccc(-c2ccc(-c3nc(-c4ccc(-c5ccccc5)cc4)nc(-c4cc5ccccc5c5c4ccc4ccccc45)n3)cc2)cc1 ; CC1(C)c2ccccc2-c2nc(-c3ccc4ccc5c6ccccc6ccc5c4c3)nc3cccc1c23 ; c1ccc(-c2nc(-c3ccc4ccc5c6ccccc6ccc5c4c3)nc3c2-c2cccc4cccc-3c24)cc1]

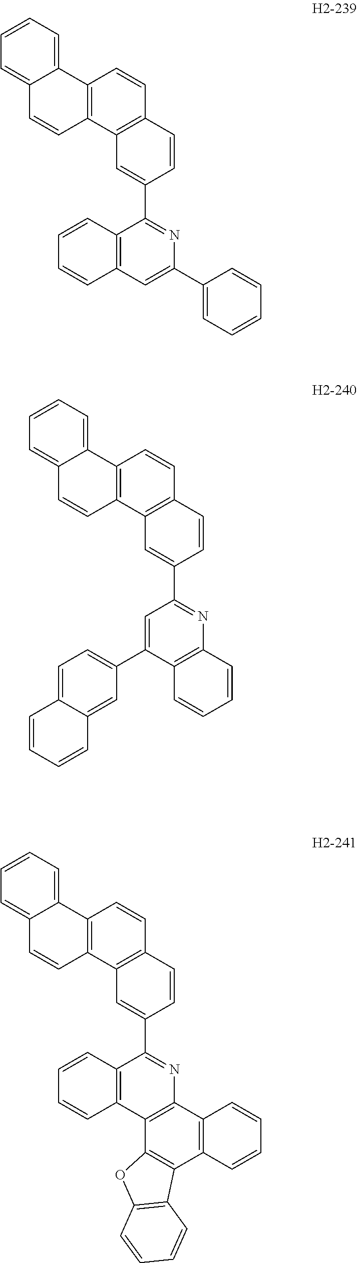 [SMILES: c1ccc(-c2cc3ccccc3c(-c3ccc4ccc5c6ccccc6ccc5c4c3)n2)cc1 ; c1ccc2cc(-c3cc(-c4ccc5ccc6c7ccccc7ccc6c5c4)nc4ccccc34)ccc2c1 ; c1ccc2c(c1)ccc1c3cc(-c4nc5c6ccccc6c6c7ccccc7oc6c5c5ccccc45)ccc3ccc21]

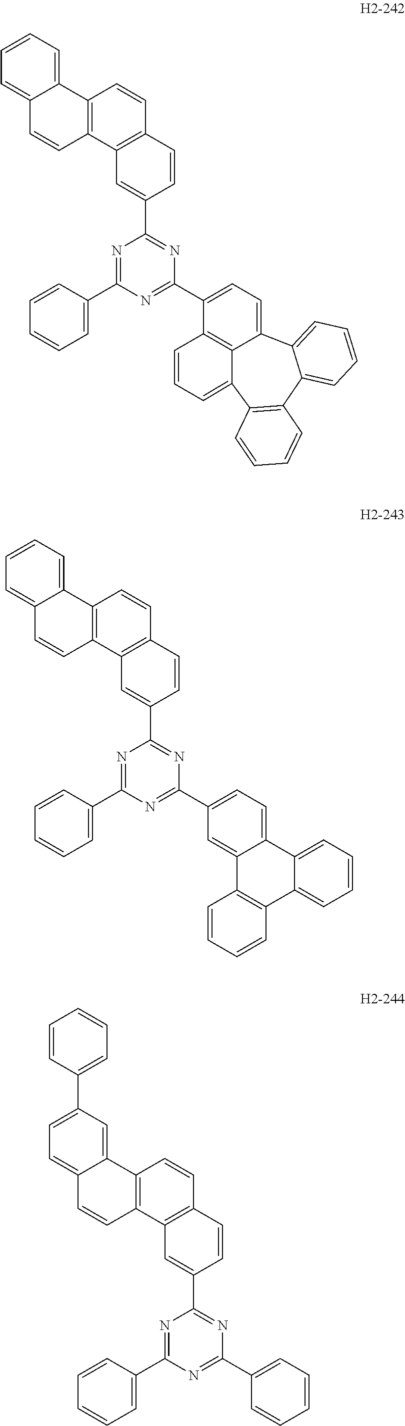 [SMILES: c1ccc(-c2nc(-c3ccc4ccc5c6ccccc6ccc5c4c3)nc(-c3ccc4c5c(cccc35)-c3ccccc3-c3ccccc3-4)n2)cc1 ; c1ccc(-c2nc(-c3ccc4ccc5c6ccccc6ccc5c4c3)nc(-c3ccc4c5ccccc5c5ccccc5c4c3)n2)cc1 ; c1ccc(-c2ccc3ccc4c5cc(-c6nc(-c7ccccc7)nc(-c7ccccc7)n6)ccc5ccc4c3c2)cc1]

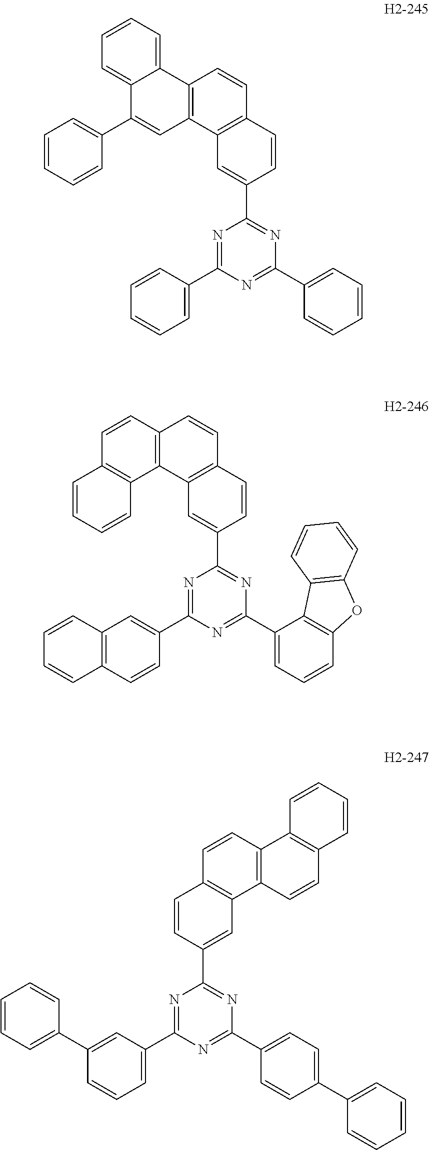 [SMILES: c1ccc(-c2nc(-c3ccccc3)nc(-c3ccc4ccc5c6ccccc6c(-c6ccccc6)cc5c4c3)n2)cc1 ; c1ccc2cc(-c3nc(-c4ccc5ccc6ccc7ccccc7c6c5c4)nc(-c4cccc5oc6ccccc6c45)n3)ccc2c1 ; c1ccc(-c2ccc(-c3nc(-c4cccc(-c5ccccc5)c4)nc(-c4ccc5ccc6c7ccccc7ccc6c5c4)n3)cc2)cc1]

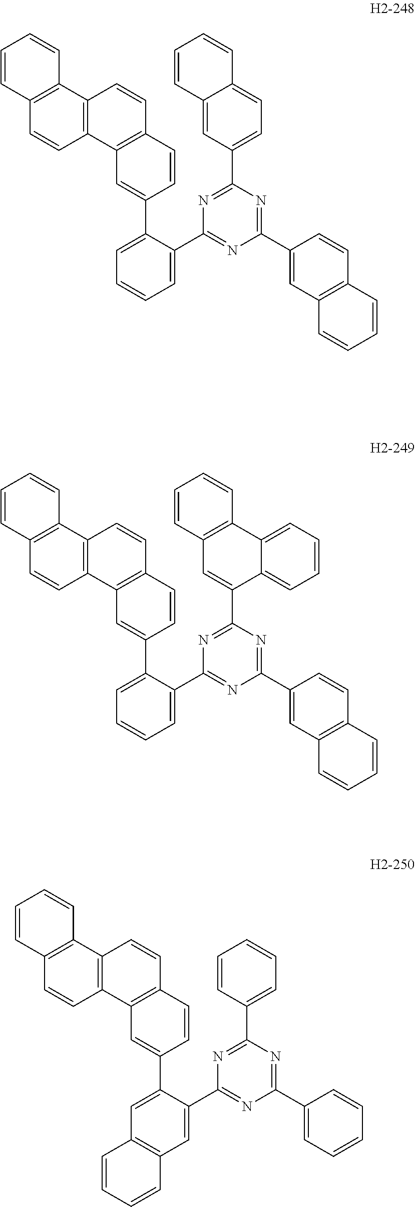 [SMILES: c1ccc(-c2nc(-c3ccc4ccccc4c3)nc(-c3ccc4ccccc4c3)n2)c(-c2ccc3ccc4c5ccccc5ccc4c3c2)c1 ; c1ccc(-c2nc(-c3ccc4ccccc4c3)nc(-c3cc4ccccc4c4ccccc34)n2)c(-c2ccc3ccc4c5ccccc5ccc4c3c2)c1 ; c1ccc(-c2nc(-c3ccccc3)nc(-c3cc4ccccc4cc3-c3ccc4ccc5c6ccccc6ccc5c4c3)n2)cc1]

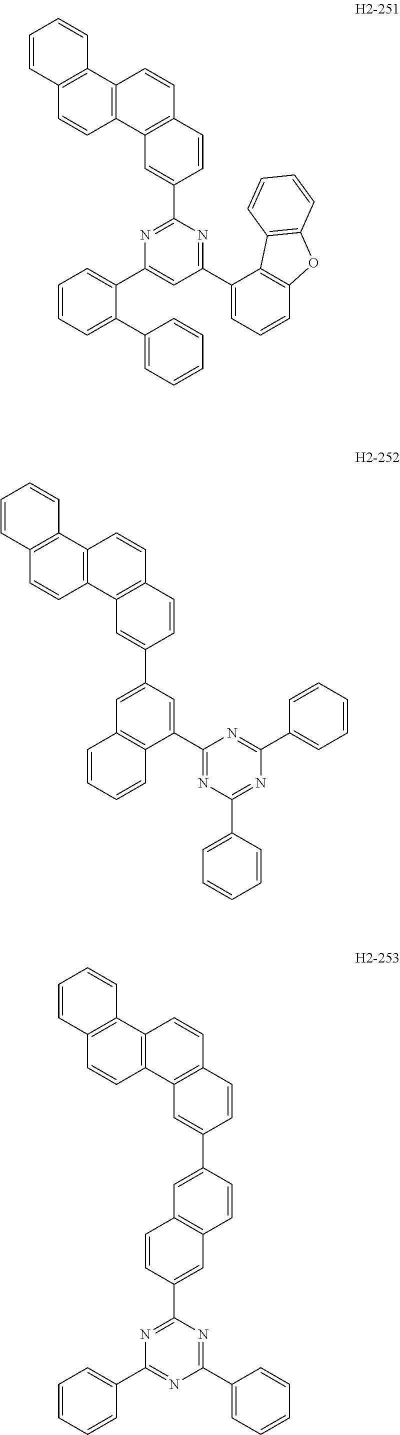 [SMILES: c1ccc(-c2ccccc2-c2cc(-c3cccc4oc5ccccc5c34)nc(-c3ccc4ccc5c6ccccc6ccc5c4c3)n2)cc1 ; c1ccc(-c2nc(-c3ccccc3)nc(-c3cc(-c4ccc5ccc6c7ccccc7ccc6c5c4)cc4ccccc34)n2)cc1 ; c1ccc(-c2nc(-c3ccccc3)nc(-c3ccc4cc(-c5ccc6ccc7c8ccccc8ccc7c6c5)ccc4c3)n2)cc1]

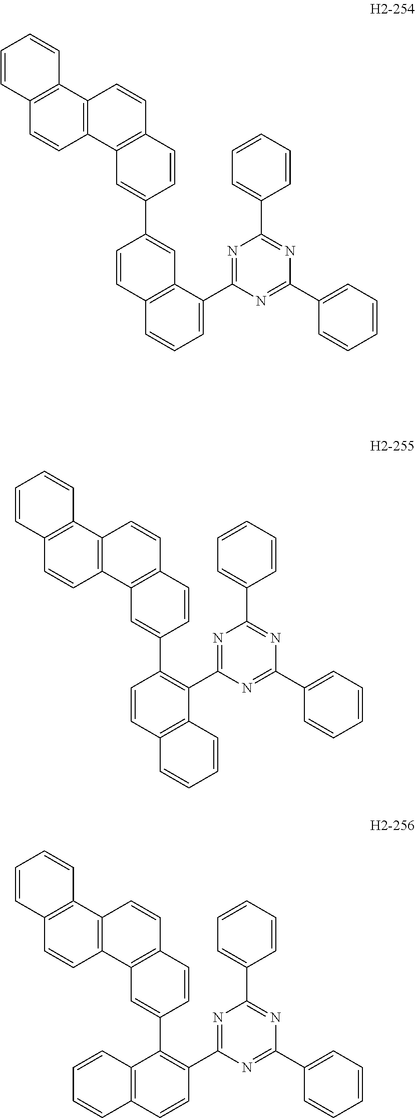 [SMILES: c1ccc(-c2nc(-c3ccccc3)nc(-c3cccc4ccc(-c5ccc6ccc7c8ccccc8ccc7c6c5)cc34)n2)cc1 ; c1ccc(-c2nc(-c3ccccc3)nc(-c3c(-c4ccc5ccc6c7ccccc7ccc6c5c4)ccc4ccccc34)n2)cc1 ; c1ccc(-c2nc(-c3ccccc3)nc(-c3ccc4ccccc4c3-c3ccc4ccc5c6ccccc6ccc5c4c3)n2)cc1]

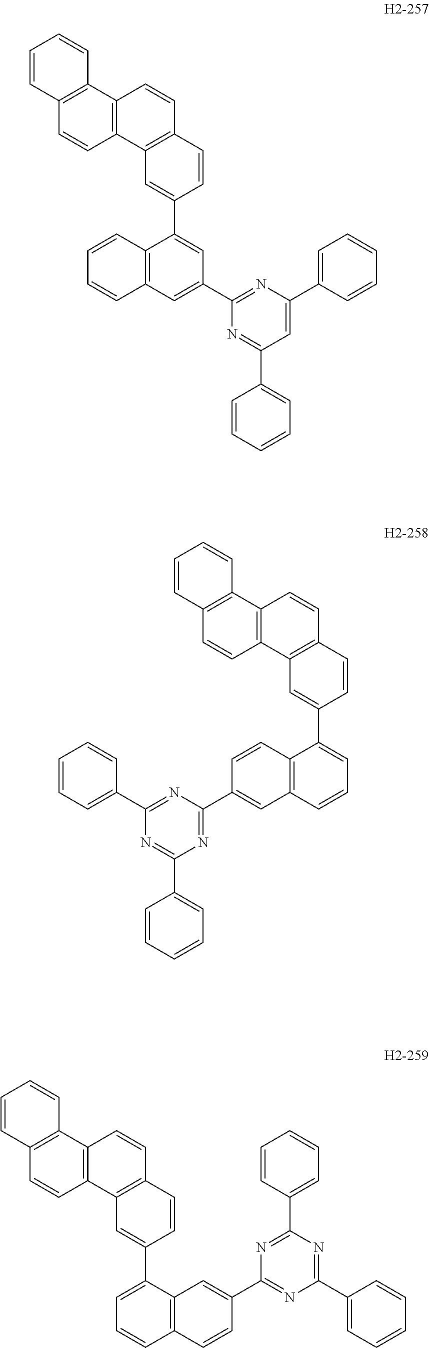 [SMILES: c1ccc(-c2cc(-c3ccccc3)nc(-c3cc(-c4ccc5ccc6c7ccccc7ccc6c5c4)c4ccccc4c3)n2)cc1 ; c1ccc(-c2nc(-c3ccccc3)nc(-c3ccc4c(-c5ccc6ccc7c8ccccc8ccc7c6c5)cccc4c3)n2)cc1 ; c1ccc(-c2nc(-c3ccccc3)nc(-c3ccc4cccc(-c5ccc6ccc7c8ccccc8ccc7c6c5)c4c3)n2)cc1]

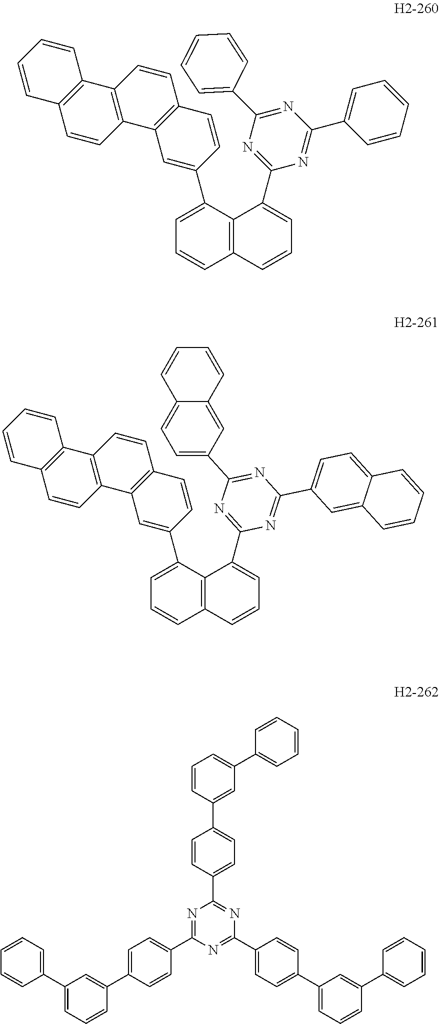 [SMILES: c1ccc(-c2nc(-c3ccccc3)nc(-c3cccc4cccc(-c5ccc6ccc7c8ccccc8ccc7c6c5)c34)n2)cc1 ; c1ccc2cc(-c3nc(-c4ccc5ccccc5c4)nc(-c4cccc5cccc(-c6ccc7ccc8c9ccccc9ccc8c7c6)c45)n3)ccc2c1 ; c1ccc(-c2cccc(-c3ccc(-c4nc(-c5ccc(-c6cccc(-c7ccccc7)c6)cc5)nc(-c5ccc(-c6cccc(-c7ccccc7)c6)cc5)n4)cc3)c2)cc1]

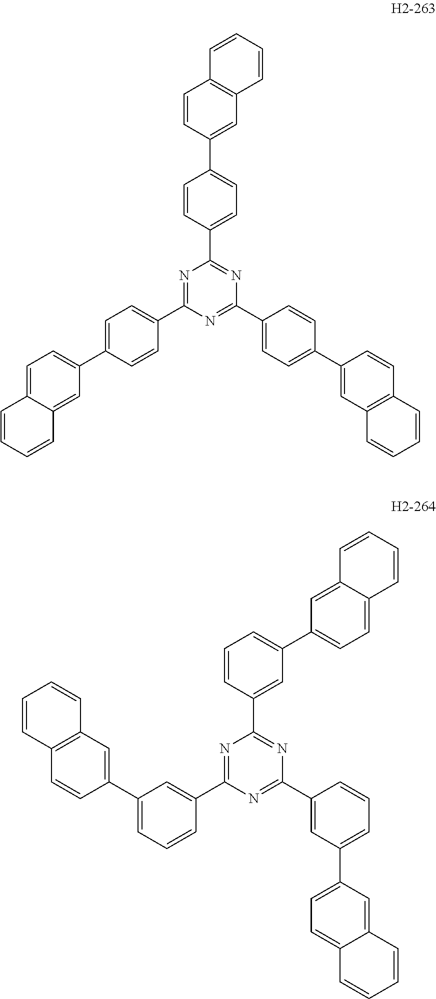 [SMILES: c1ccc2cc(-c3ccc(-c4nc(-c5ccc(-c6ccc7ccccc7c6)cc5)nc(-c5ccc(-c6ccc7ccccc7c6)cc5)n4)cc3)ccc2c1 ; c1cc(-c2ccc3ccccc3c2)cc(-c2nc(-c3cccc(-c4ccc5ccccc5c4)c3)nc(-c3cccc(-c4ccc5ccccc5c4)c3)n2)c1]

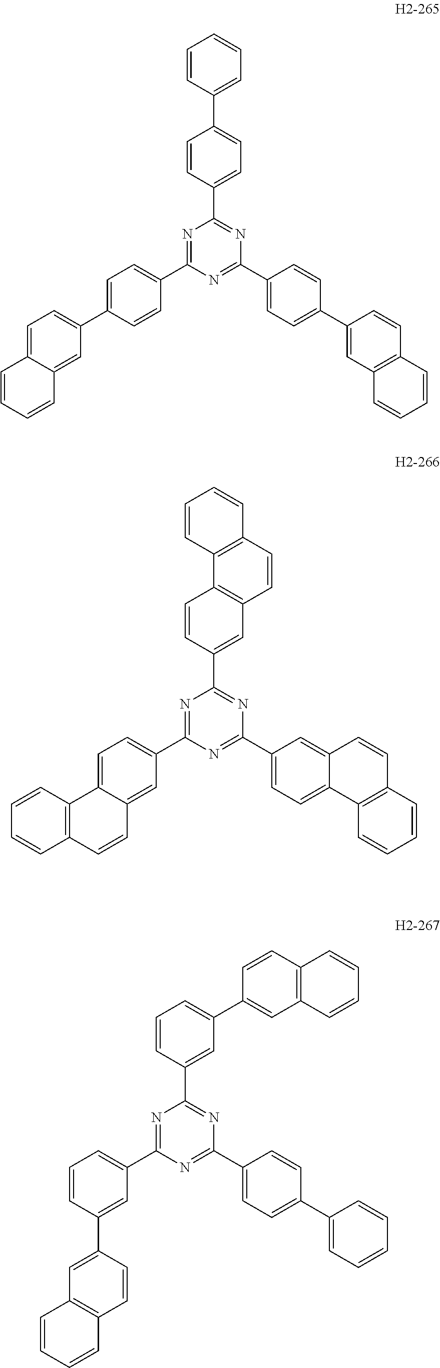 [SMILES: c1ccc(-c2ccc(-c3nc(-c4ccc(-c5ccc6ccccc6c5)cc4)nc(-c4ccc(-c5ccc6ccccc6c5)cc4)n3)cc2)cc1 ; c1ccc2c(c1)ccc1cc(-c3nc(-c4ccc5c(ccc6ccccc65)c4)nc(-c4ccc5c(ccc6ccccc65)c4)n3)ccc12 ; c1ccc(-c2ccc(-c3nc(-c4cccc(-c5ccc6ccccc6c5)c4)nc(-c4cccc(-c5ccc6ccccc6c5)c4)n3)cc2)cc1]

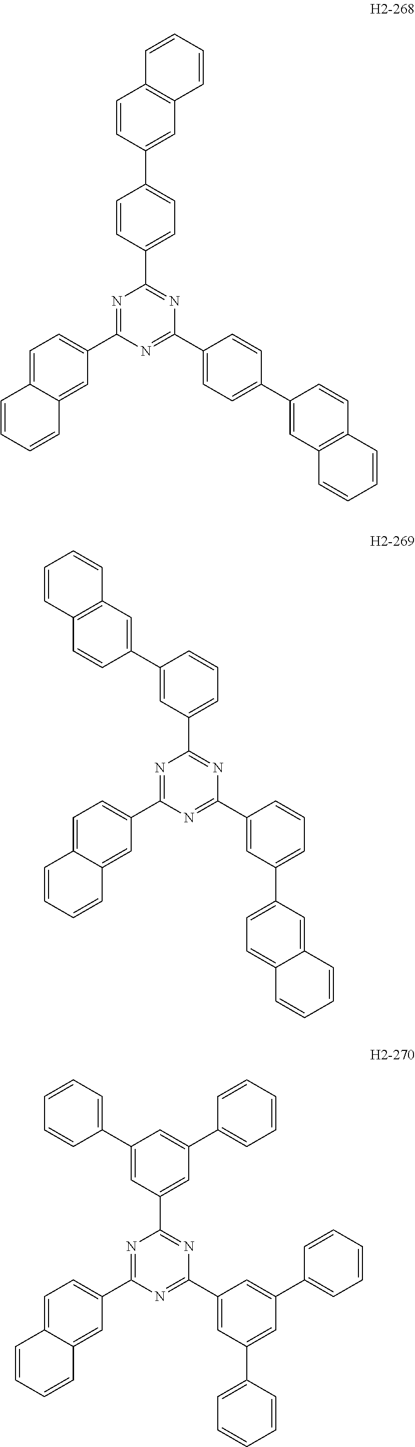 [SMILES: c1ccc2cc(-c3ccc(-c4nc(-c5ccc(-c6ccc7ccccc7c6)cc5)nc(-c5ccc6ccccc6c5)n4)cc3)ccc2c1 ; c1cc(-c2ccc3ccccc3c2)cc(-c2nc(-c3cccc(-c4ccc5ccccc5c4)c3)nc(-c3ccc4ccccc4c3)n2)c1 ; c1ccc(-c2cc(-c3ccccc3)cc(-c3nc(-c4cc(-c5ccccc5)cc(-c5ccccc5)c4)nc(-c4ccc5ccccc5c4)n3)c2)cc1]

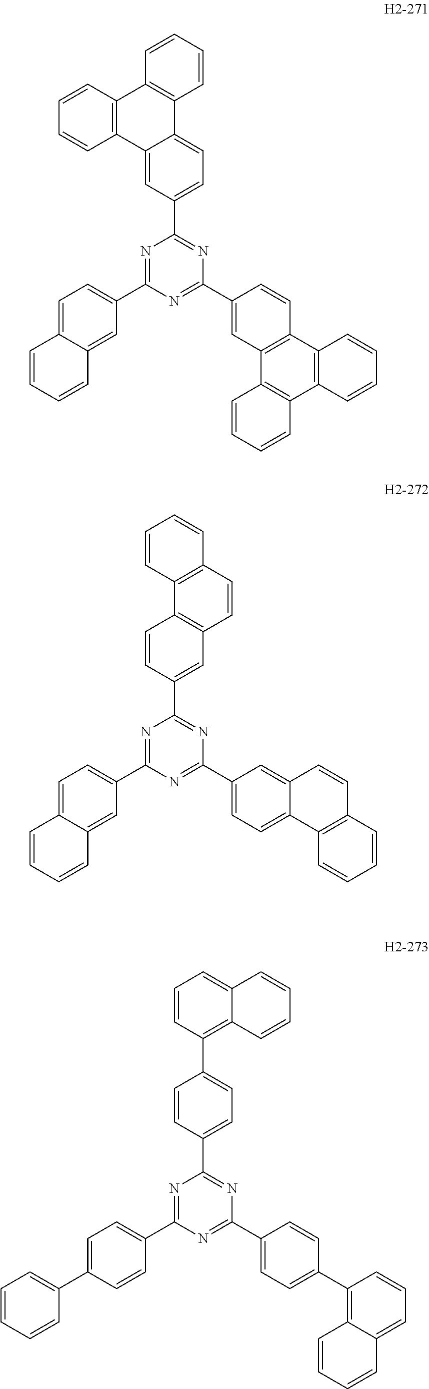 [SMILES: c1ccc2cc(-c3nc(-c4ccc5c6ccccc6c6ccccc6c5c4)nc(-c4ccc5c6ccccc6c6ccccc6c5c4)n3)ccc2c1 ; c1ccc2cc(-c3nc(-c4ccc5c(ccc6ccccc65)c4)nc(-c4ccc5c(ccc6ccccc65)c4)n3)ccc2c1 ; c1ccc(-c2ccc(-c3nc(-c4ccc(-c5cccc6ccccc56)cc4)nc(-c4ccc(-c5cccc6ccccc56)cc4)n3)cc2)cc1]

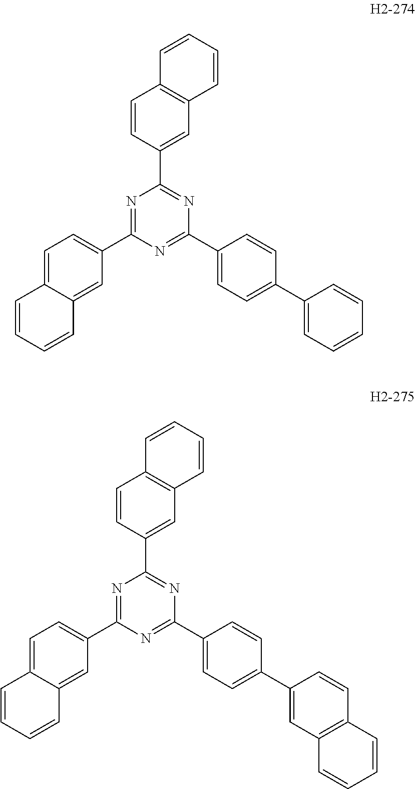 [SMILES: c1ccc(-c2ccc(-c3nc(-c4ccc5ccccc5c4)nc(-c4ccc5ccccc5c4)n3)cc2)cc1 ; c1ccc2cc(-c3ccc(-c4nc(-c5ccc6ccccc6c5)nc(-c5ccc6ccccc6c5)n4)cc3)ccc2c1]

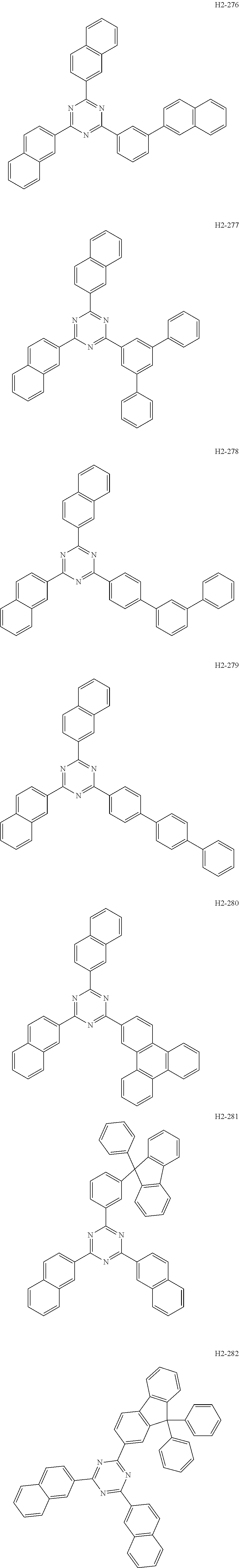

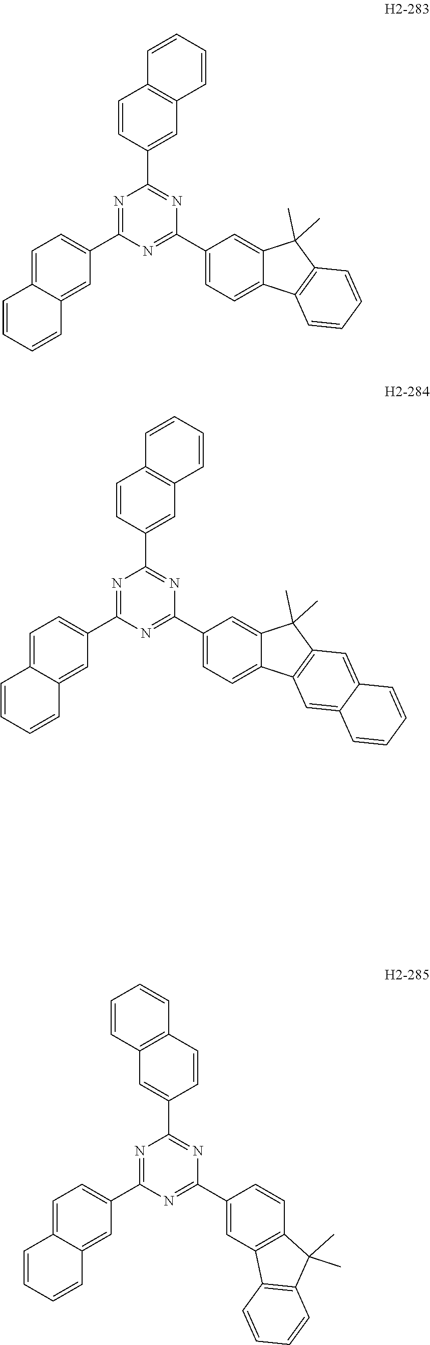 [SMILES: CC1(C)c2ccccc2-c2ccc(-c3nc(-c4ccc5ccccc5c4)nc(-c4ccc5ccccc5c4)n3)cc21 ; CC1(C)c2cc(-c3nc(-c4ccc5ccccc5c4)nc(-c4ccc5ccccc5c4)n3)ccc2-c2cc3ccccc3cc21 ; CC1(C)c2ccccc2-c2cc(-c3nc(-c4ccc5ccccc5c4)nc(-c4ccc5ccccc5c4)n3)ccc21]

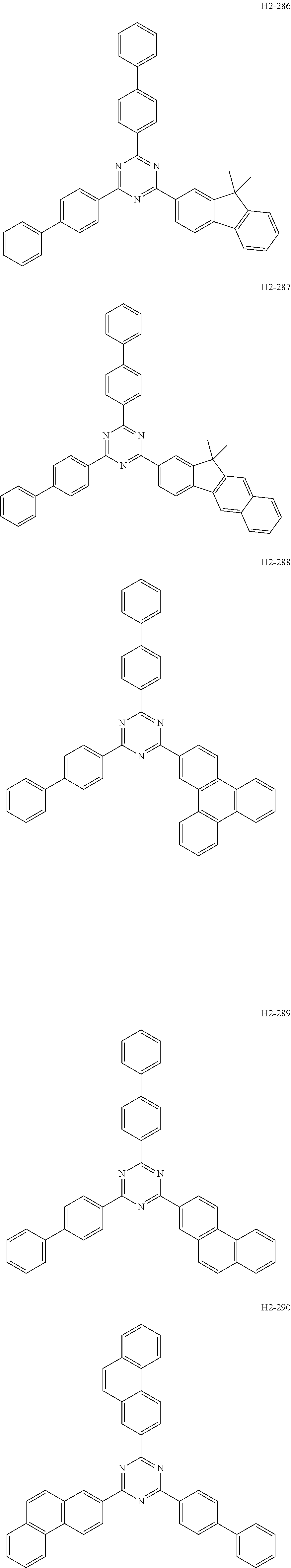 [SMILES: CC1(C)c2ccccc2-c2ccc(-c3nc(-c4ccc(-c5ccccc5)cc4)nc(-c4ccc(-c5ccccc5)cc4)n3)cc21 ; CC1(C)c2cc(-c3nc(-c4ccc(-c5ccccc5)cc4)nc(-c4ccc(-c5ccccc5)cc4)n3)ccc2-c2cc3ccccc3cc21 ; c1ccc(-c2ccc(-c3nc(-c4ccc(-c5ccccc5)cc4)nc(-c4ccc5c6ccccc6c6ccccc6c5c4)n3)cc2)cc1 ; c1ccc(-c2ccc(-c3nc(-c4ccc(-c5ccccc5)cc4)nc(-c4ccc5c(ccc6ccccc65)c4)n3)cc2)cc1 ; c1ccc(-c2ccc(-c3nc(-c4ccc5c(ccc6ccccc65)c4)nc(-c4ccc5c(ccc6ccccc65)c4)n3)cc2)cc1]

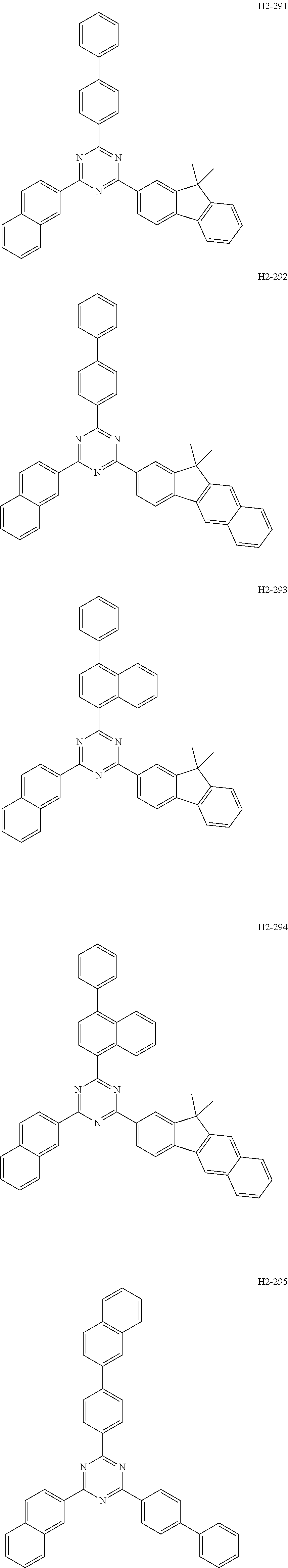 [SMILES: CC1(C)c2ccccc2-c2ccc(-c3nc(-c4ccc(-c5ccccc5)cc4)nc(-c4ccc5ccccc5c4)n3)cc21 ; CC1(C)c2cc(-c3nc(-c4ccc(-c5ccccc5)cc4)nc(-c4ccc5ccccc5c4)n3)ccc2-c2cc3ccccc3cc21 ; CC1(C)c2ccccc2-c2ccc(-c3nc(-c4ccc5ccccc5c4)nc(-c4ccc(-c5ccccc5)c5ccccc45)n3)cc21 ; CC1(C)c2cc(-c3nc(-c4ccc5ccccc5c4)nc(-c4ccc(-c5ccccc5)c5ccccc45)n3)ccc2-c2cc3ccccc3cc21 ; c1ccc(-c2ccc(-c3nc(-c4ccc(-c5ccc6ccccc6c5)cc4)nc(-c4ccc5ccccc5c4)n3)cc2)cc1]

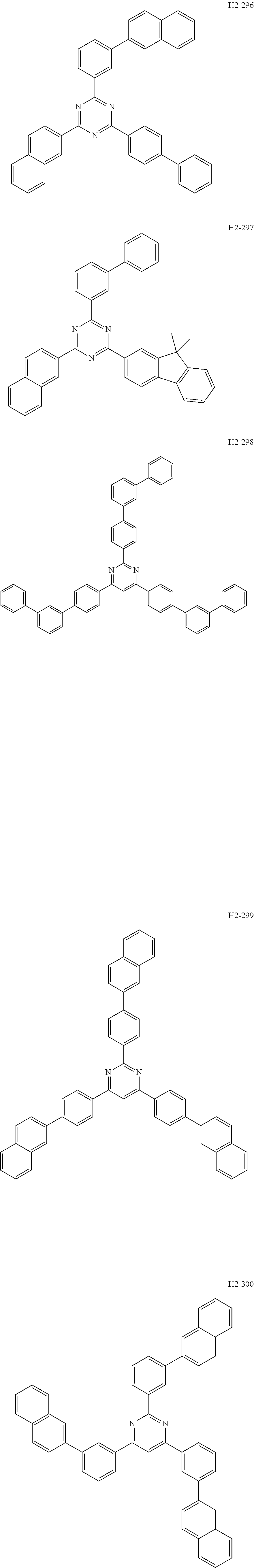 [SMILES: c1ccc(-c2ccc(-c3nc(-c4cccc(-c5ccc6ccccc6c5)c4)nc(-c4ccc5ccccc5c4)n3)cc2)cc1 ; CC1(C)c2ccccc2-c2ccc(-c3nc(-c4cccc(-c5ccccc5)c4)nc(-c4ccc5ccccc5c4)n3)cc21 ; c1ccc(-c2cccc(-c3ccc(-c4cc(-c5ccc(-c6cccc(-c7ccccc7)c6)cc5)nc(-c5ccc(-c6cccc(-c7ccccc7)c6)cc5)n4)cc3)c2)cc1 ; c1ccc2cc(-c3ccc(-c4cc(-c5ccc(-c6ccc7ccccc7c6)cc5)nc(-c5ccc(-c6ccc7ccccc7c6)cc5)n4)cc3)ccc2c1 ; c1cc(-c2ccc3ccccc3c2)cc(-c2cc(-c3cccc(-c4ccc5ccccc5c4)c3)nc(-c3cccc(-c4ccc5ccccc5c4)c3)n2)c1]

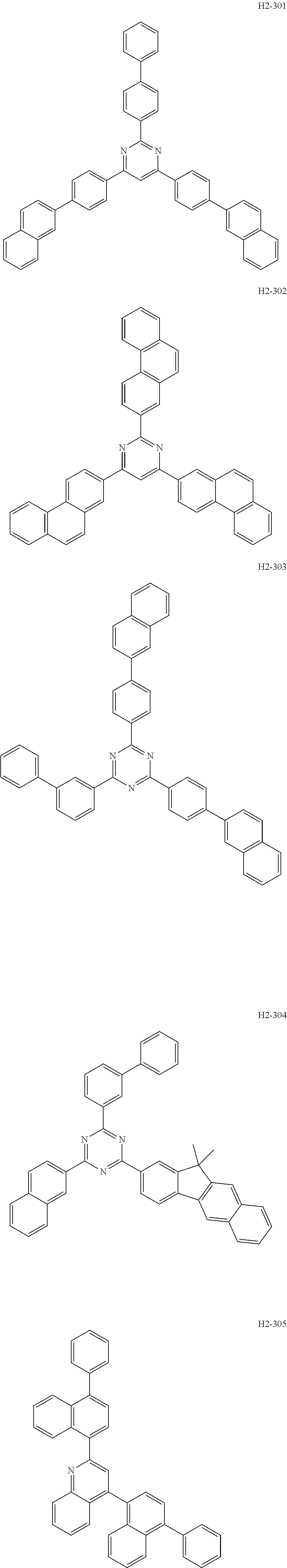 [SMILES: c1ccc(-c2ccc(-c3nc(-c4ccc(-c5ccc6ccccc6c5)cc4)cc(-c4ccc(-c5ccc6ccccc6c5)cc4)n3)cc2)cc1 ; c1ccc2c(c1)ccc1cc(-c3cc(-c4ccc5c(ccc6ccccc65)c4)nc(-c4ccc5c(ccc6ccccc65)c4)n3)ccc12 ; c1ccc(-c2cccc(-c3nc(-c4ccc(-c5ccc6ccccc6c5)cc4)nc(-c4ccc(-c5ccc6ccccc6c5)cc4)n3)c2)cc1 ; CC1(C)c2cc(-c3nc(-c4cccc(-c5ccccc5)c4)nc(-c4ccc5ccccc5c4)n3)ccc2-c2cc3ccccc3cc21 ; c1ccc(-c2ccc(-c3cc(-c4ccc(-c5ccccc5)c5ccccc45)c4ccccc4n3)c3ccccc23)cc1]

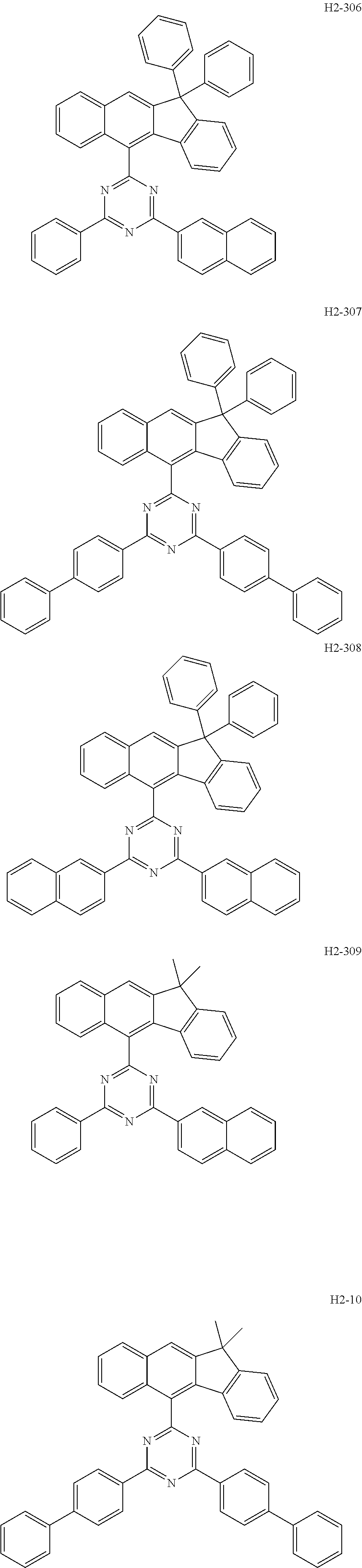 [SMILES: c1ccc(-c2nc(-c3ccc4ccccc4c3)nc(-c3c4c(cc5ccccc35)C(c3ccccc3)(c3ccccc3)c3ccccc3-4)n2)cc1 ; c1ccc(-c2ccc(-c3nc(-c4ccc(-c5ccccc5)cc4)nc(-c4c5c(cc6ccccc46)C(c4ccccc4)(c4ccccc4)c4ccccc4-5)n3)cc2)cc1 ; c1ccc(C2(c3ccccc3)c3ccccc3-c3c2cc2ccccc2c3-c2nc(-c3ccc4ccccc4c3)nc(-c3ccc4ccccc4c3)n2)cc1 ; CC1(C)c2ccccc2-c2c1cc1ccccc1c2-c1nc(-c2ccccc2)nc(-c2ccc3ccccc3c2)n1 ; CC1(C)c2ccccc2-c2c1cc1ccccc1c2-c1nc(-c2ccc(-c3ccccc3)cc2)nc(-c2ccc(-c3ccccc3)cc2)n1]

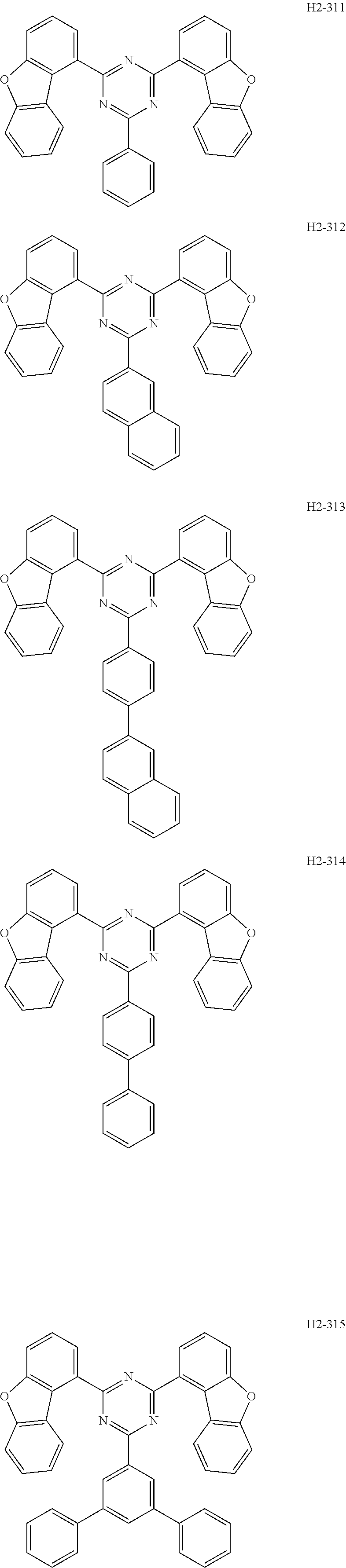 [SMILES: c1ccc(-c2nc(-c3cccc4oc5ccccc5c34)nc(-c3cccc4oc5ccccc5c34)n2)cc1 ; c1ccc2cc(-c3nc(-c4cccc5oc6ccccc6c45)nc(-c4cccc5oc6ccccc6c45)n3)ccc2c1 ; c1ccc2cc(-c3ccc(-c4nc(-c5cccc6oc7ccccc7c56)nc(-c5cccc6oc7ccccc7c56)n4)cc3)ccc2c1 ; c1ccc(-c2ccc(-c3nc(-c4cccc5oc6ccccc6c45)nc(-c4cccc5oc6ccccc6c45)n3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1]

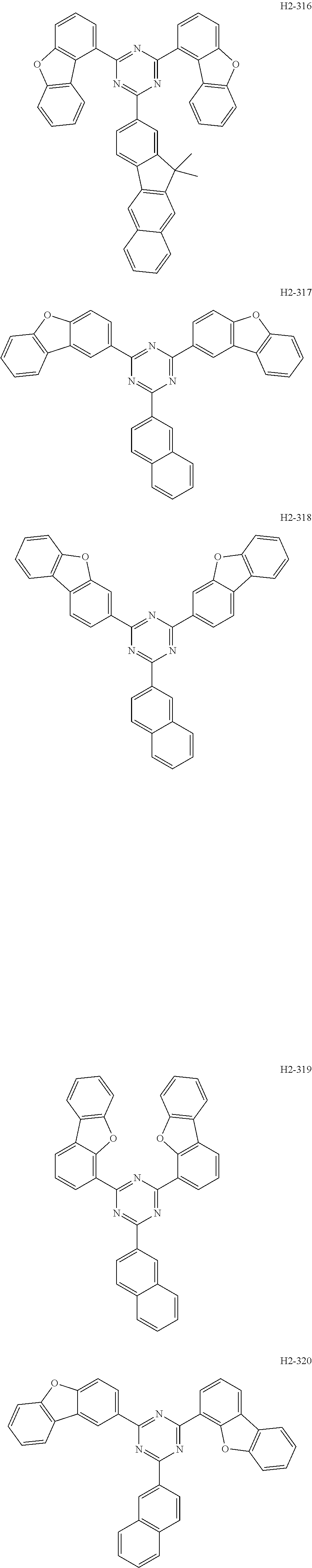 [SMILES: CC1(C)c2cc(-c3nc(-c4cccc5oc6ccccc6c45)nc(-c4cccc5oc6ccccc6c45)n3)ccc2-c2cc3ccccc3cc21 ; c1ccc2cc(-c3nc(-c4ccc5oc6ccccc6c5c4)nc(-c4ccc5oc6ccccc6c5c4)n3)ccc2c1 ; c1ccc2cc(-c3nc(-c4ccc5c(c4)oc4ccccc45)nc(-c4ccc5c(c4)oc4ccccc45)n3)ccc2c1 ; c1ccc2cc(-c3nc(-c4cccc5c4oc4ccccc45)nc(-c4cccc5c4oc4ccccc45)n3)ccc2c1 ; c1ccc2cc(-c3nc(-c4ccc5oc6ccccc6c5c4)nc(-c4cccc5c4oc4ccccc45)n3)ccc2c1]

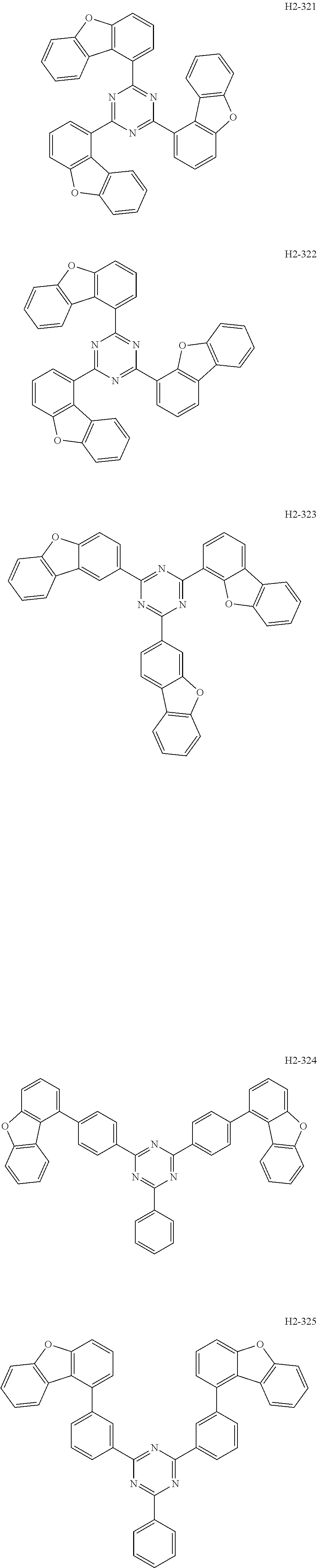 [SMILES: c1ccc2c(c1)oc1cccc(-c3nc(-c4cccc5oc6ccccc6c45)nc(-c4cccc5oc6ccccc6c45)n3)c12 ; c1ccc2c(c1)oc1c(-c3nc(-c4cccc5oc6ccccc6c45)nc(-c4cccc5oc6ccccc6c45)n3)cccc12 ; c1ccc2c(c1)oc1cc(-c3nc(-c4ccc5oc6ccccc6c5c4)nc(-c4cccc5c4oc4ccccc45)n3)ccc12 ; c1ccc(-c2nc(-c3ccc(-c4cccc5oc6ccccc6c45)cc3)nc(-c3ccc(-c4cccc5oc6ccccc6c45)cc3)n2)cc1 ; c1ccc(-c2nc(-c3cccc(-c4cccc5oc6ccccc6c45)c3)nc(-c3cccc(-c4cccc5oc6ccccc6c45)c3)n2)cc1]

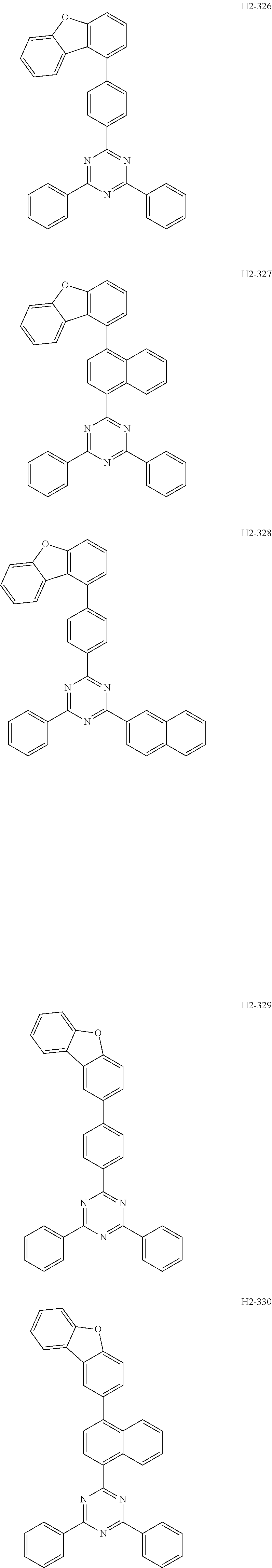 [SMILES: c1ccc(-c2nc(-c3ccccc3)nc(-c3ccc(-c4cccc5oc6ccccc6c45)cc3)n2)cc1 ; c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1 ; c1ccc(-c2nc(-c3ccccc3)nc(-c3ccc(-c4ccc5oc6ccccc6c5c4)cc3)n2)cc1 ; c1ccc(-c2nc(-c3ccccc3)nc(-c3ccc(-c4ccc5oc6ccccc6c5c4)c4ccccc34)n2)cc1]

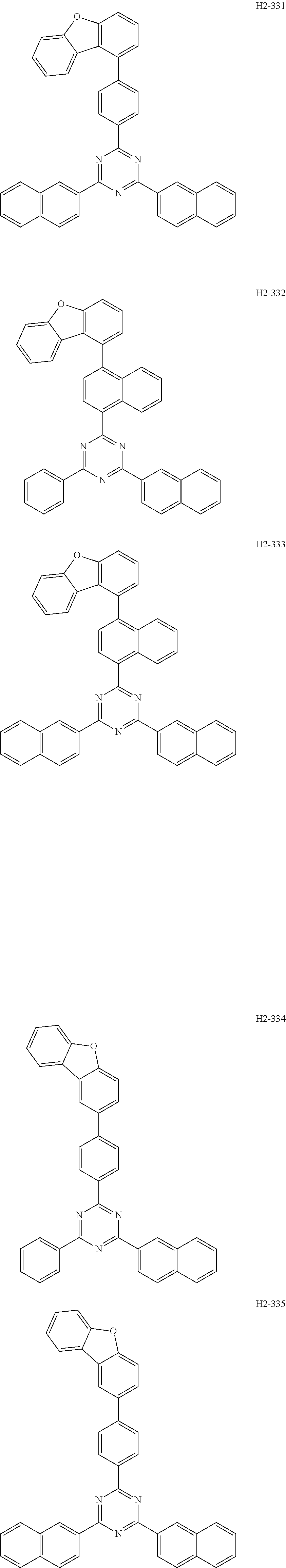 [SMILES: c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1 ; c1ccc2cc(-c3nc(-c4ccc5ccccc5c4)nc(-c4ccc(-c5cccc6oc7ccccc7c56)c5ccccc45)n3)ccc2c1 ; c1ccc(-c2nc(-c3ccc(-c4ccc5oc6ccccc6c5c4)cc3)nc(-c3ccc4ccccc4c3)n2)cc1 ; c1ccc2cc(-c3nc(-c4ccc(-c5ccc6oc7ccccc7c6c5)cc4)nc(-c4ccc5ccccc5c4)n3)ccc2c1]

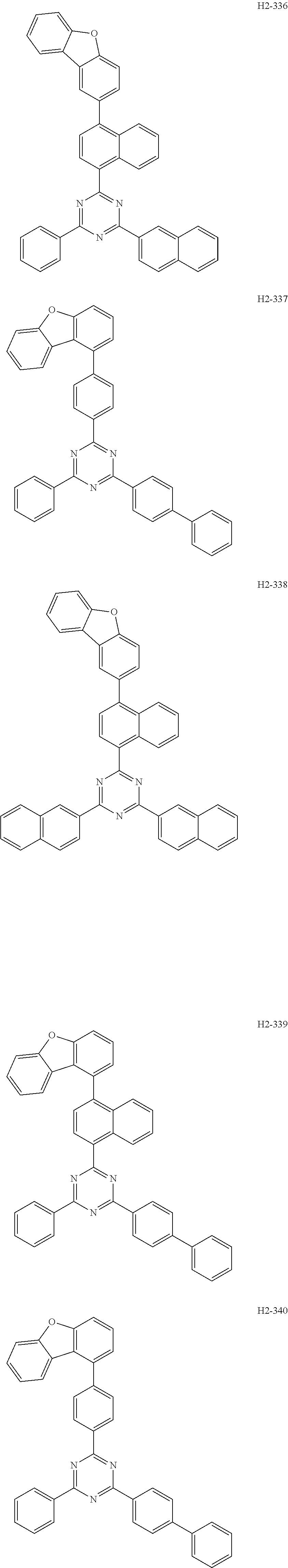 [SMILES: c1ccc(-c2nc(-c3ccc4ccccc4c3)nc(-c3ccc(-c4ccc5oc6ccccc6c5c4)c4ccccc34)n2)cc1 ; c1ccc(-c2ccc(-c3nc(-c4ccccc4)nc(-c4ccc(-c5cccc6oc7ccccc7c56)cc4)n3)cc2)cc1 ; c1ccc2cc(-c3nc(-c4ccc5ccccc5c4)nc(-c4ccc(-c5ccc6oc7ccccc7c6c5)c5ccccc45)n3)ccc2c1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2ccc(-c3nc(-c4ccccc4)nc(-c4ccc(-c5cccc6oc7ccccc7c56)cc4)n3)cc2)cc1]

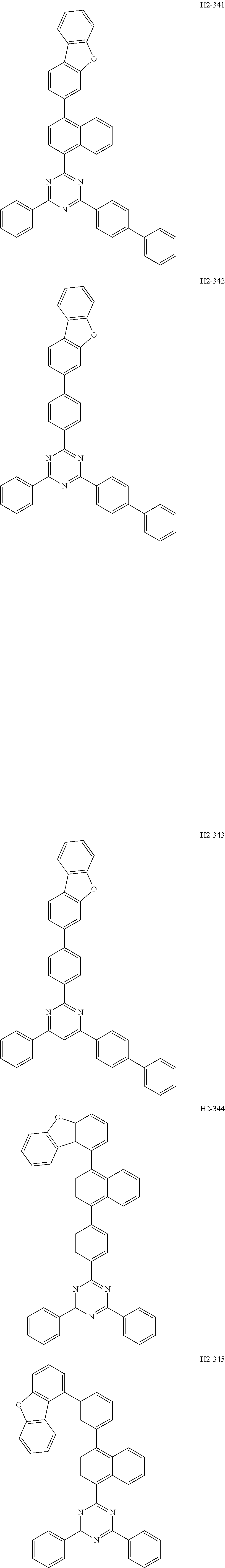

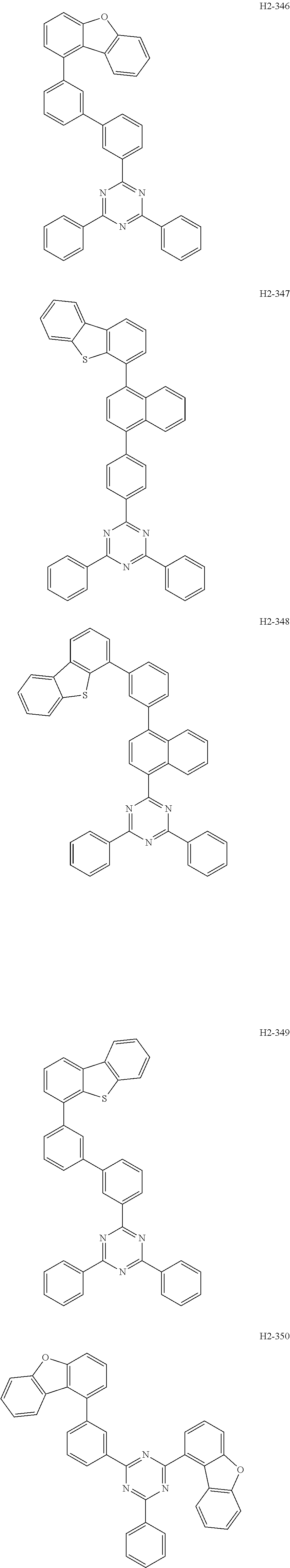 [SMILES: c1ccc(-c2nc(-c3ccccc3)nc(-c3cccc(-c4cccc(-c5cccc6oc7ccccc7c56)c4)c3)n2)cc1 ; c1ccc(-c2nc(-c3ccccc3)nc(-c3ccc(-c4ccc(-c5cccc6c5sc5ccccc56)c5ccccc45)cc3)n2)cc1 ; c1ccc(-c2nc(-c3ccccc3)nc(-c3ccc(-c4cccc(-c5cccc6c5sc5ccccc56)c4)c4ccccc34)n2)cc1 ; c1ccc(-c2nc(-c3ccccc3)nc(-c3cccc(-c4cccc(-c5cccc6c5sc5ccccc56)c4)c3)n2)cc1 ; c1ccc(-c2nc(-c3cccc(-c4cccc5oc6ccccc6c45)c3)nc(-c3cccc4oc5ccccc5c34)n2)cc1]

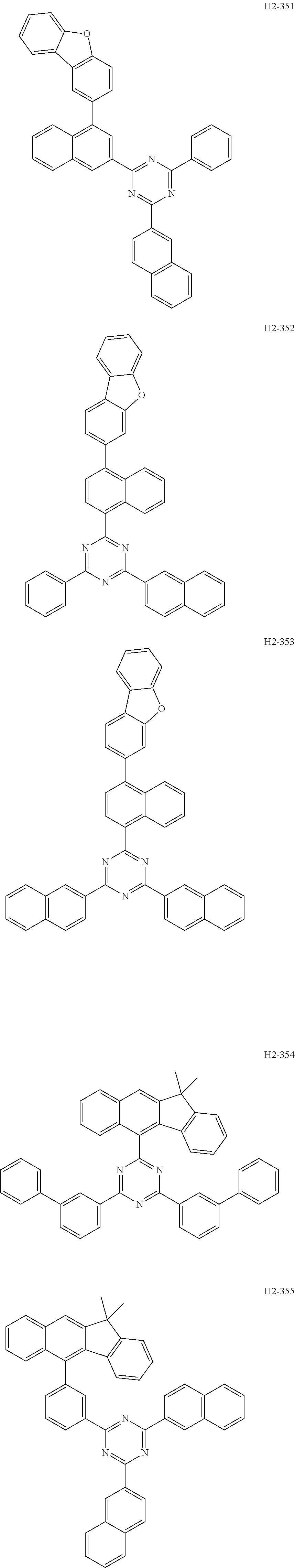 [SMILES: c1ccc(-c2nc(-c3ccc4ccccc4c3)nc(-c3cc(-c4ccc5oc6ccccc6c5c4)c4ccccc4c3)n2)cc1 ; c1ccc(-c2nc(-c3ccc4ccccc4c3)nc(-c3ccc(-c4ccc5c(c4)oc4ccccc45)c4ccccc34)n2)cc1 ; c1ccc2cc(-c3nc(-c4ccc5ccccc5c4)nc(-c4ccc(-c5ccc6c(c5)oc5ccccc56)c5ccccc45)n3)ccc2c1 ; CC1(C)c2ccccc2-c2c1cc1ccccc1c2-c1nc(-c2cccc(-c3ccccc3)c2)nc(-c2cccc(-c3ccccc3)c2)n1 ; CC1(C)c2ccccc2-c2c1cc1ccccc1c2-c1cccc(-c2nc(-c3ccc4ccccc4c3)nc(-c3ccc4ccccc4c3)n2)c1]

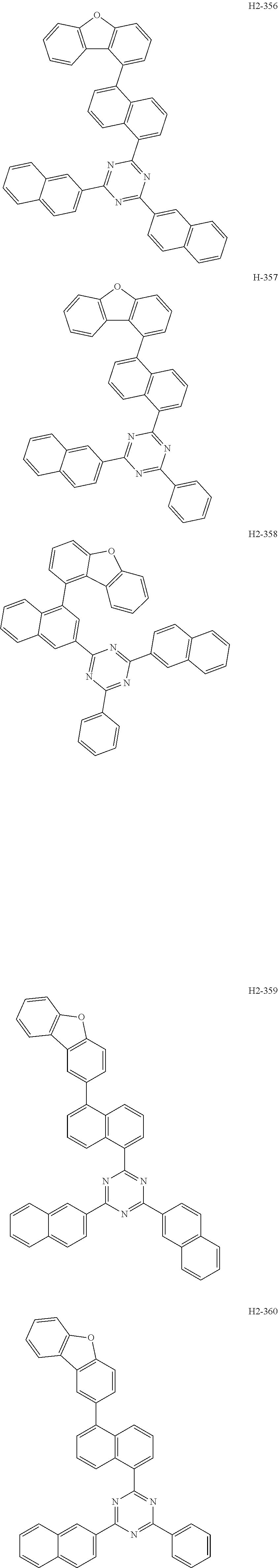 [SMILES: c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1 ; c1ccc2cc(-c3nc(-c4ccc5ccccc5c4)nc(-c4cccc5c(-c6ccc7oc8ccccc8c7c6)cccc45)n3)ccc2c1 ; c1ccc(-c2nc(-c3ccc4ccccc4c3)nc(-c3cccc4c(-c5ccc6oc7ccccc7c6c5)cccc34)n2)cc1]

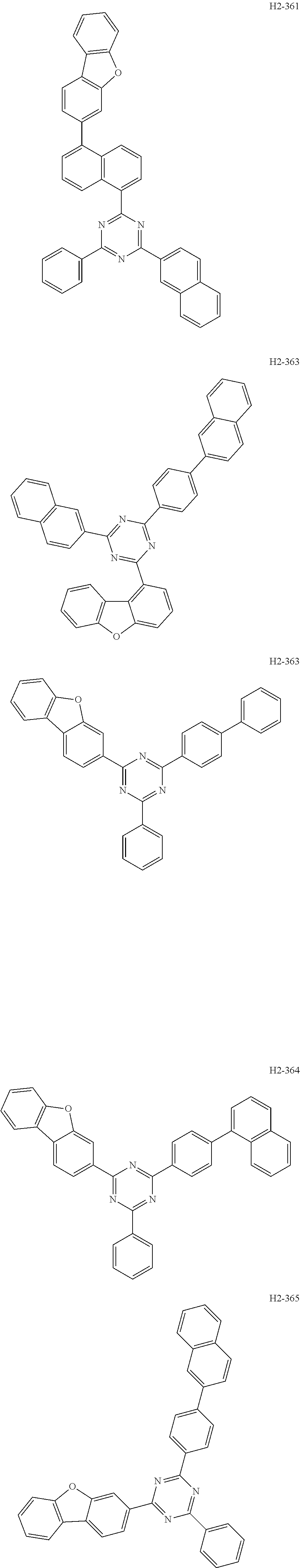 [SMILES: c1ccc(-c2nc(-c3ccc4ccccc4c3)nc(-c3cccc4c(-c5ccc6c(c5)oc5ccccc56)cccc34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccccc4)nc(-c4ccc5c(c4)oc4ccccc45)n3)cc2)cc1 ; c1ccc(-c2nc(-c3ccc(-c4cccc5ccccc45)cc3)nc(-c3ccc4c(c3)oc3ccccc34)n2)cc1 ; c1ccc(-c2nc(-c3ccc(-c4ccc5ccccc5c4)cc3)nc(-c3ccc4c(c3)oc3ccccc34)n2)cc1]

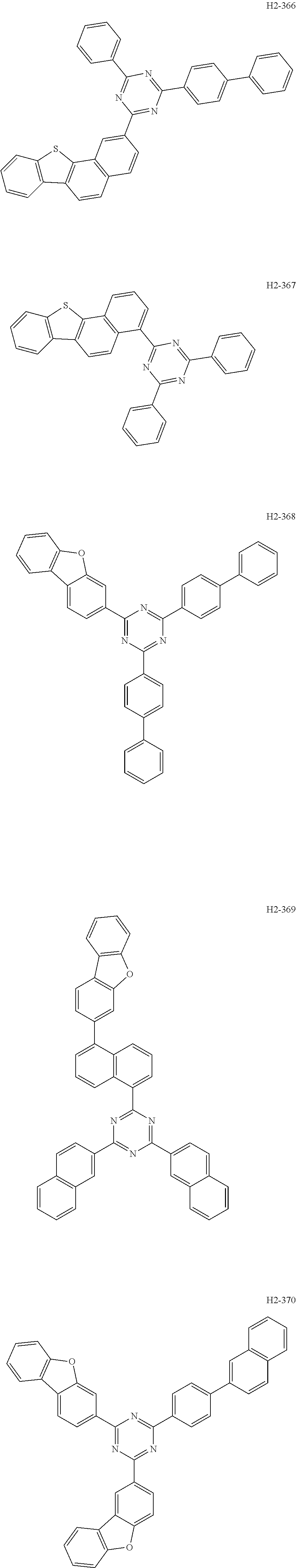 [SMILES: c1ccc(-c2ccc(-c3nc(-c4ccccc4)nc(-c4ccc5ccc6c7ccccc7sc6c5c4)n3)cc2)cc1 ; c1ccc(-c2nc(-c3ccccc3)nc(-c3cccc4c3ccc3c5ccccc5sc43)n2)cc1 ; c1ccc(-c2ccc(-c3nc(-c4ccc(-c5ccccc5)cc4)nc(-c4ccc5c(c4)oc4ccccc45)n3)cc2)cc1 ; c1ccc2cc(-c3nc(-c4ccc5ccccc5c4)nc(-c4cccc5c(-c6ccc7c(c6)oc6ccccc67)cccc45)n3)ccc2c1 ; c1ccc2cc(-c3ccc(-c4nc(-c5ccc6c(c5)oc5ccccc56)nc(-c5ccc6oc7ccccc7c6c5)n4)cc3)ccc2c1]

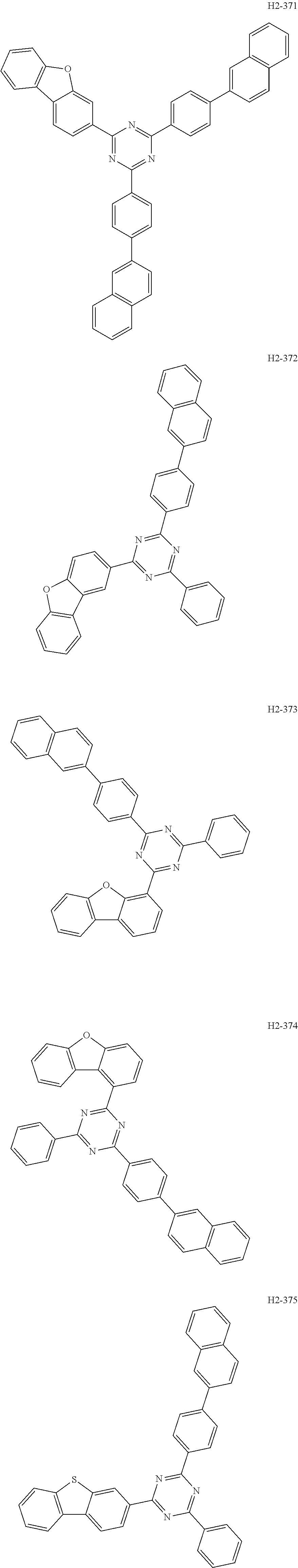 [SMILES: c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1 ; c1ccc(-c2nc(-c3ccc(-c4ccc5ccccc5c4)cc3)nc(-c3cccc4c3oc3ccccc34)n2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc4c(c3)sc3ccccc34)n2)cc1]

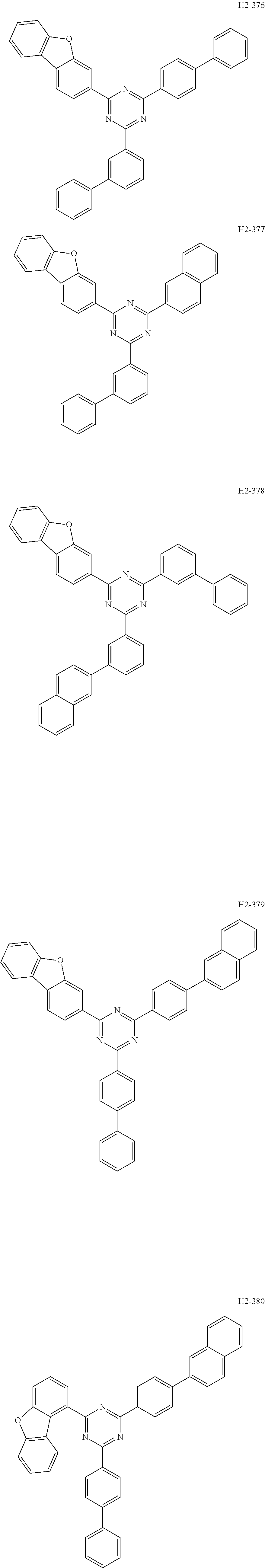 [SMILES: c1ccc(-c2ccc(-c3nc(-c4cccc(-c5ccccc5)c4)nc(-c4ccc5c(c4)oc4ccccc45)n3)cc2)cc1 ; c1ccc(-c2cccc(-c3nc(-c4ccc5ccccc5c4)nc(-c4ccc5c(c4)oc4ccccc45)n3)c2)cc1 ; c1ccc(-c2cccc(-c3nc(-c4cccc(-c5ccc6ccccc6c5)c4)nc(-c4ccc5c(c4)oc4ccccc45)n3)c2)cc1 ; c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2ccc(-c3nc(-c4ccc(-c5ccc6ccccc6c5)cc4)nc(-c4cccc5oc6ccccc6c45)n3)cc2)cc1]

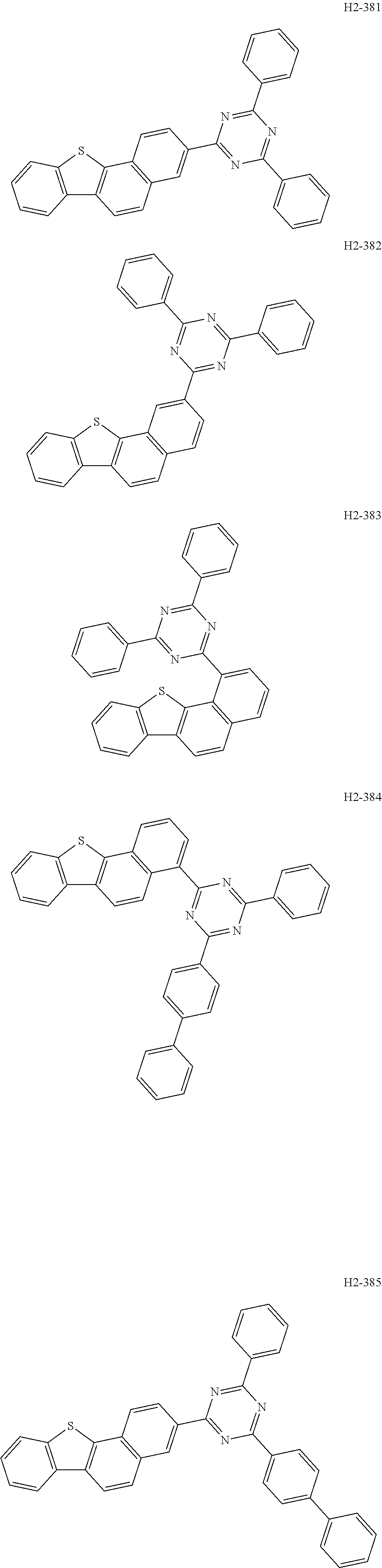 [SMILES: c1ccc(-c2nc(-c3ccccc3)nc(-c3ccc4c(ccc5c6ccccc6sc45)c3)n2)cc1 ; c1ccc(-c2nc(-c3ccccc3)nc(-c3ccc4ccc5c6ccccc6sc5c4c3)n2)cc1 ; c1ccc(-c2nc(-c3ccccc3)nc(-c3cccc4ccc5c6ccccc6sc5c34)n2)cc1 ; c1ccc(-c2ccc(-c3nc(-c4ccccc4)nc(-c4cccc5c4ccc4c6ccccc6sc54)n3)cc2)cc1 ; c1ccc(-c2ccc(-c3nc(-c4ccccc4)nc(-c4ccc5c(ccc6c7ccccc7sc56)c4)n3)cc2)cc1]

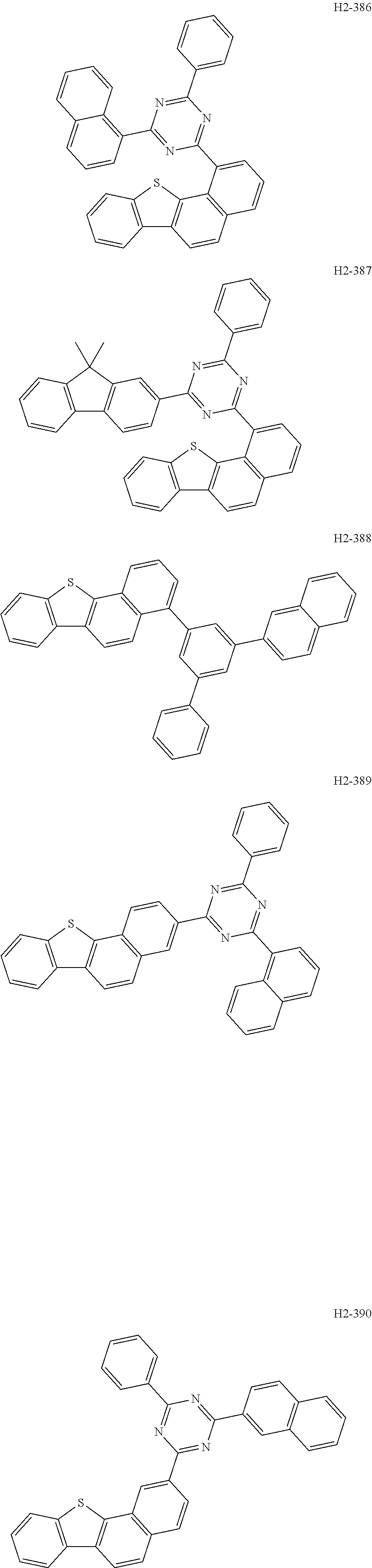 [SMILES: c1ccc(-c2nc(-c3cccc4ccccc34)nc(-c3cccc4ccc5c6ccccc6sc5c34)n2)cc1 ; CC1(C)c2ccccc2-c2ccc(-c3nc(-c4ccccc4)nc(-c4cccc5ccc6c7ccccc7sc6c45)n3)cc21 ; c1ccc(-c2cc(-c3ccc4ccccc4c3)cc(-c3cccc4c3ccc3c5ccccc5sc43)c2)cc1 ; c1ccc(-c2nc(-c3ccc4c(ccc5c6ccccc6sc45)c3)nc(-c3cccc4ccccc34)n2)cc1 ; c1ccc(-c2nc(-c3ccc4ccccc4c3)nc(-c3ccc4ccc5c6ccccc6sc5c4c3)n2)cc1]

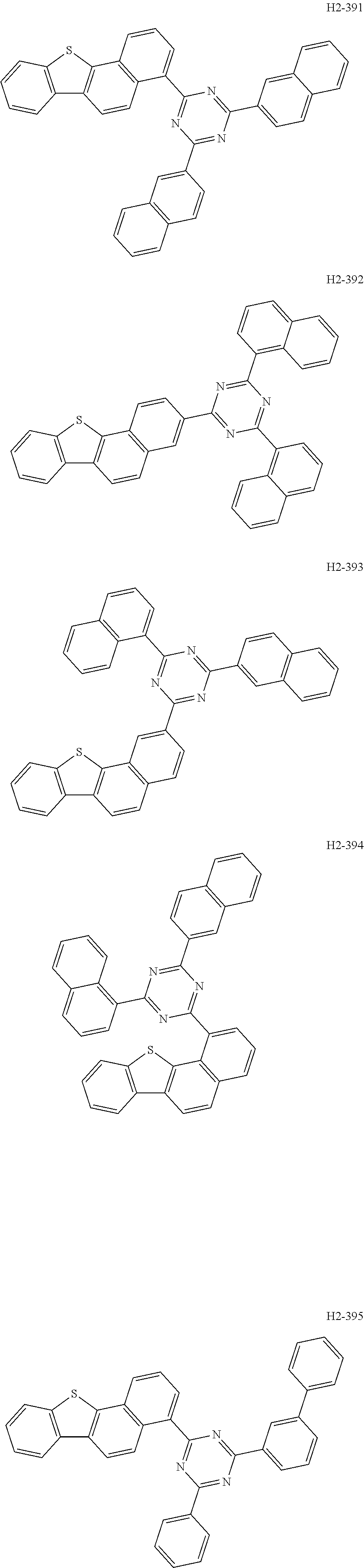 [SMILES: c1ccc2cc(-c3nc(-c4ccc5ccccc5c4)nc(-c4cccc5c4ccc4c6ccccc6sc54)n3)ccc2c1 ; c1ccc2c(-c3nc(-c4ccc5c(ccc6c7ccccc7sc56)c4)nc(-c4cccc5ccccc45)n3)cccc2c1 ; c1ccc2cc(-c3nc(-c4ccc5ccc6c7ccccc7sc6c5c4)nc(-c4cccc5ccccc45)n3)ccc2c1 ; c1ccc2cc(-c3nc(-c4cccc5ccccc45)nc(-c4cccc5ccc6c7ccccc7sc6c45)n3)ccc2c1 ; c1ccc(-c2cccc(-c3nc(-c4ccccc4)nc(-c4cccc5c4ccc4c6ccccc6sc54)n3)c2)cc1]

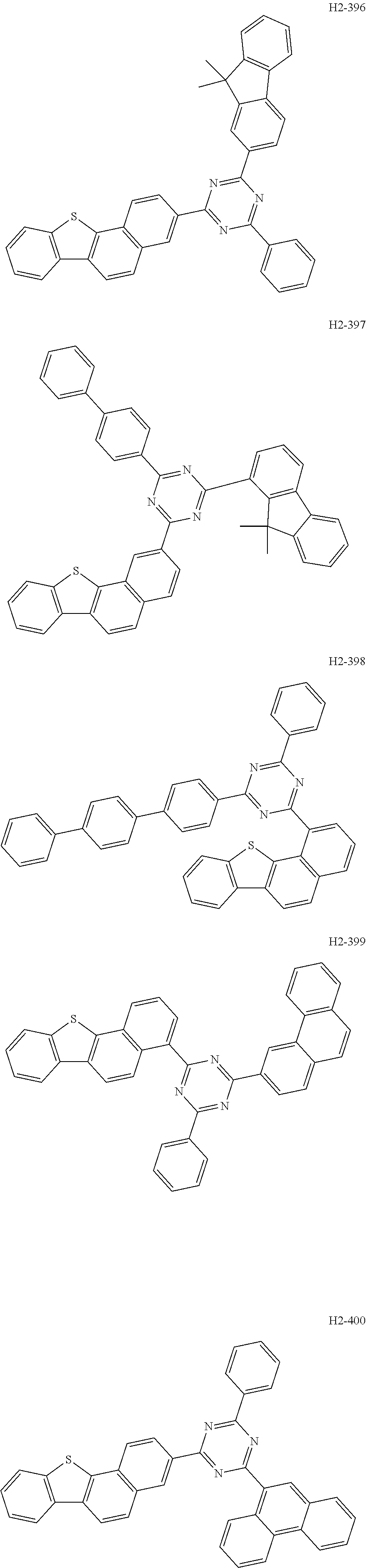 [SMILES: CC1(C)c2ccccc2-c2ccc(-c3nc(-c4ccccc4)nc(-c4ccc5c(ccc6c7ccccc7sc56)c4)n3)cc21 ; CC1(C)c2ccccc2-c2cccc(-c3nc(-c4ccc(-c5ccccc5)cc4)nc(-c4ccc5ccc6c7ccccc7sc6c5c4)n3)c21 ; c1ccc(-c2ccc(-c3ccc(-c4nc(-c5ccccc5)nc(-c5cccc6ccc7c8ccccc8sc7c56)n4)cc3)cc2)cc1 ; c1ccc(-c2nc(-c3ccc4ccc5ccccc5c4c3)nc(-c3cccc4c3ccc3c5ccccc5sc43)n2)cc1 ; c1ccc(-c2nc(-c3ccc4c(ccc5c6ccccc6sc45)c3)nc(-c3cc4ccccc4c4ccccc34)n2)cc1]

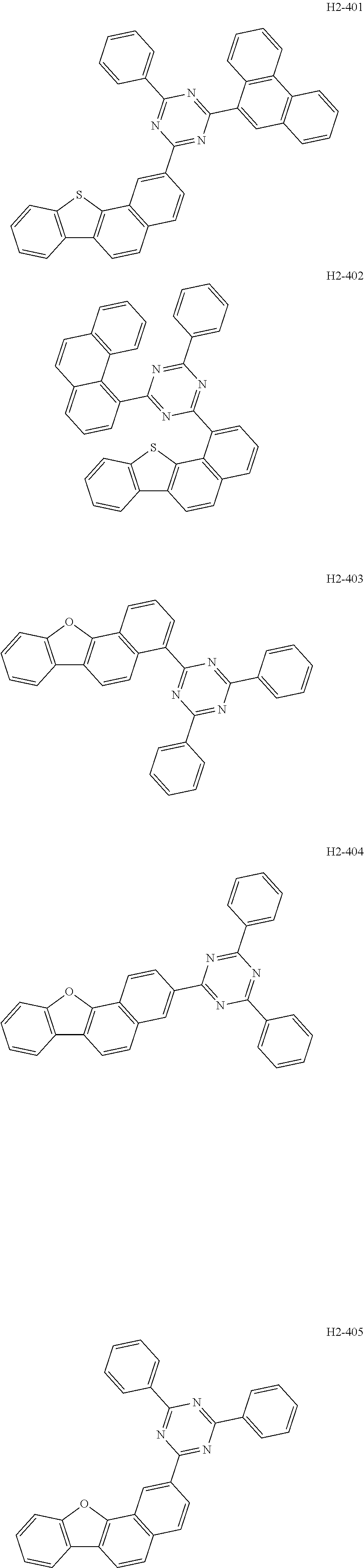 [SMILES: c1ccc(-c2nc(-c3ccc4ccc5c6ccccc6sc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6sc5c34)n2)cc1 ; c1ccc(-c2nc(-c3ccccc3)nc(-c3cccc4c3ccc3c5ccccc5oc43)n2)cc1 ; c1ccc(-c2nc(-c3ccccc3)nc(-c3ccc4c(ccc5c6ccccc6oc45)c3)n2)cc1 ; c1ccc(-c2nc(-c3ccccc3)nc(-c3ccc4ccc5c6ccccc6oc5c4c3)n2)cc1]

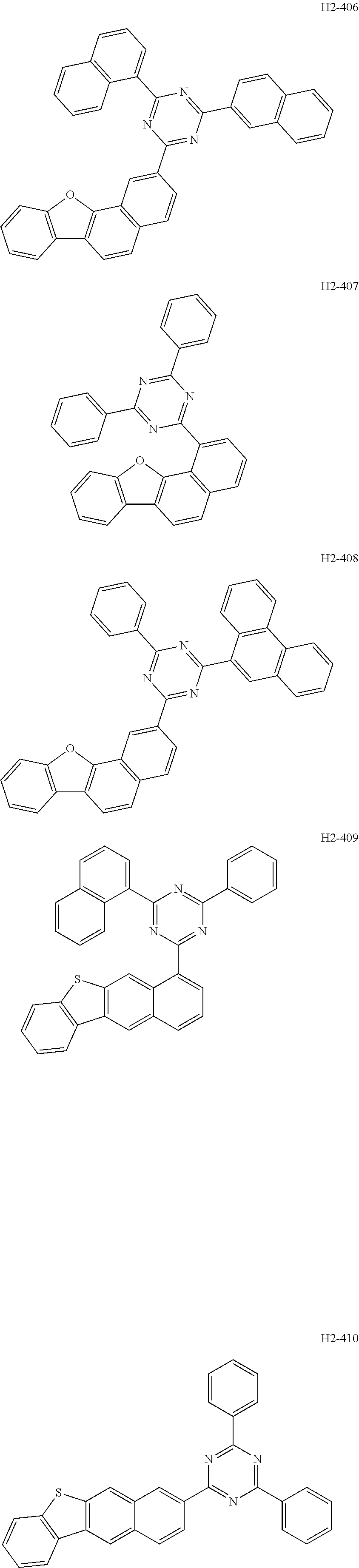

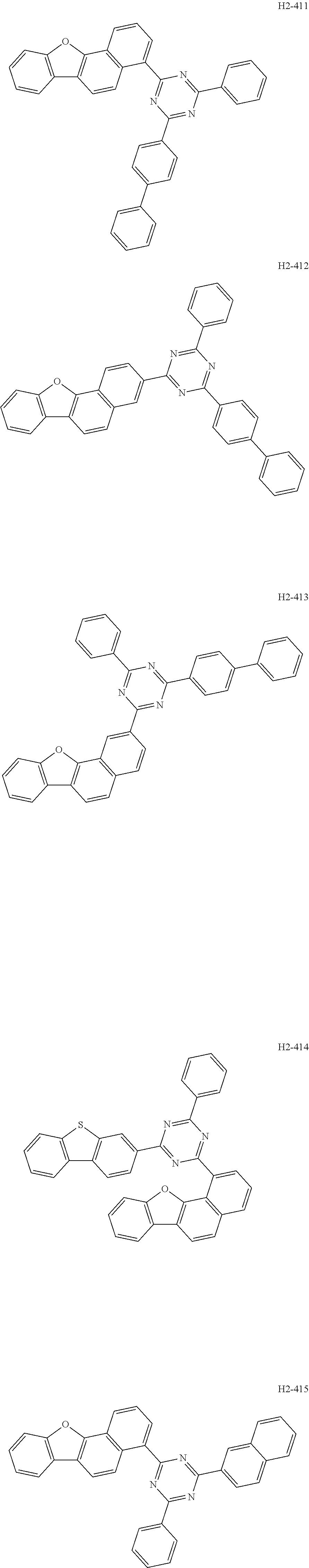 [SMILES: c1ccc(-c2ccc(-c3nc(-c4ccccc4)nc(-c4cccc5c4ccc4c6ccccc6oc54)n3)cc2)cc1 ; c1ccc(-c2ccc(-c3nc(-c4ccccc4)nc(-c4ccc5c(ccc6c7ccccc7oc56)c4)n3)cc2)cc1 ; c1ccc(-c2ccc(-c3nc(-c4ccccc4)nc(-c4ccc5ccc6c7ccccc7oc6c5c4)n3)cc2)cc1 ; c1ccc(-c2nc(-c3ccc4c(c3)sc3ccccc34)nc(-c3cccc4ccc5c6ccccc6oc5c34)n2)cc1 ; c1ccc(-c2nc(-c3ccc4ccccc4c3)nc(-c3cccc4c3ccc3c5ccccc5oc43)n2)cc1]

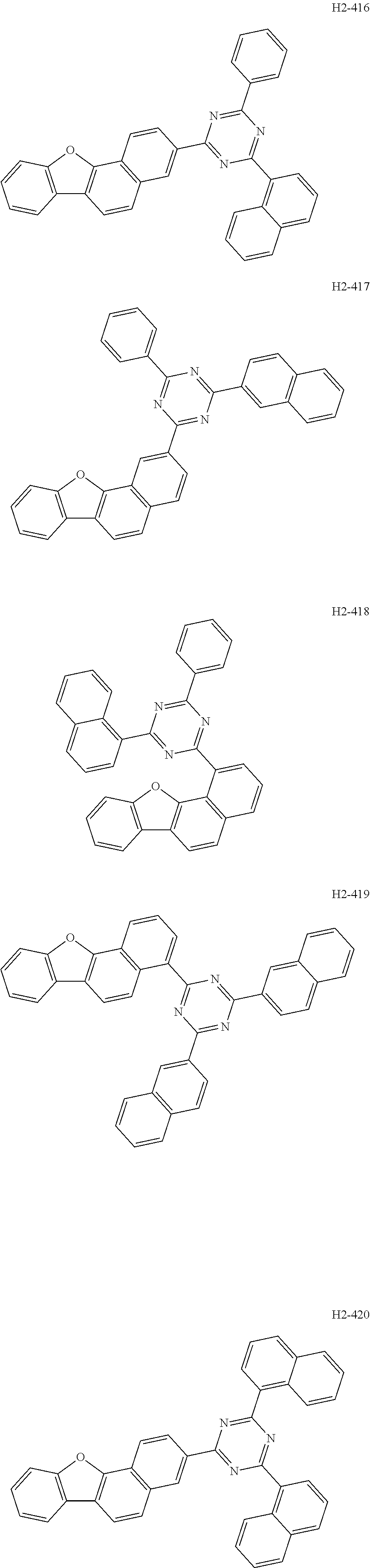 [SMILES: c1ccc(-c2nc(-c3ccc4c(ccc5c6ccccc6oc45)c3)nc(-c3cccc4ccccc34)n2)cc1 ; c1ccc(-c2nc(-c3ccc4ccccc4c3)nc(-c3ccc4ccc5c6ccccc6oc5c4c3)n2)cc1 ; c1ccc(-c2nc(-c3cccc4ccccc34)nc(-c3cccc4ccc5c6ccccc6oc5c34)n2)cc1 ; c1ccc2cc(-c3nc(-c4ccc5ccccc5c4)nc(-c4cccc5c4ccc4c6ccccc6oc54)n3)ccc2c1 ; c1ccc2c(-c3nc(-c4ccc5c(ccc6c7ccccc7oc56)c4)nc(-c4cccc5ccccc45)n3)cccc2c1]

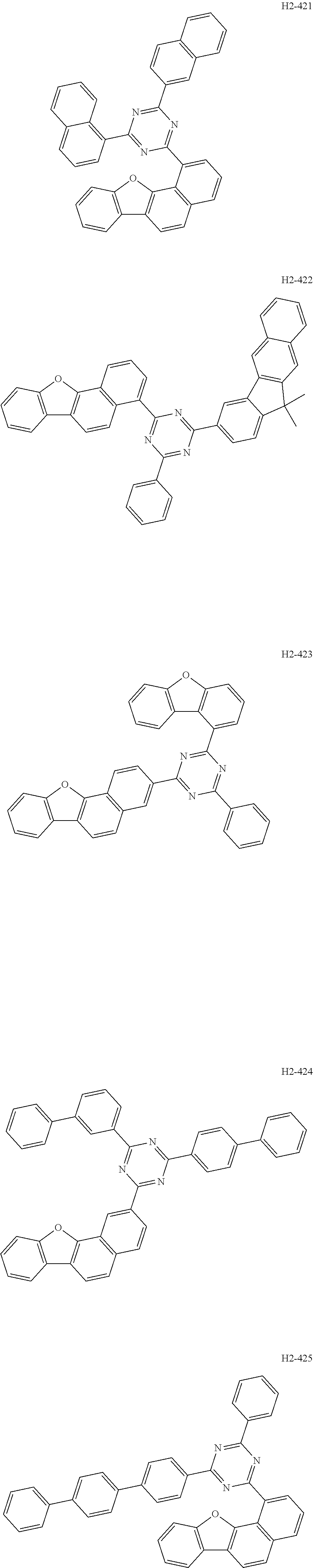 [SMILES: c1ccc2cc(-c3nc(-c4cccc5ccccc45)nc(-c4cccc5ccc6c7ccccc7oc6c45)n3)ccc2c1 ; CC1(C)c2ccc(-c3nc(-c4ccccc4)nc(-c4cccc5c4ccc4c6ccccc6oc54)n3)cc2-c2cc3ccccc3cc21 ; c1ccc(-c2nc(-c3ccc4c(ccc5c6ccccc6oc45)c3)nc(-c3cccc4oc5ccccc5c34)n2)cc1 ; c1ccc(-c2ccc(-c3nc(-c4cccc(-c5ccccc5)c4)nc(-c4ccc5ccc6c7ccccc7oc6c5c4)n3)cc2)cc1 ; c1ccc(-c2ccc(-c3ccc(-c4nc(-c5ccccc5)nc(-c5cccc6ccc7c8ccccc8oc7c56)n4)cc3)cc2)cc1]

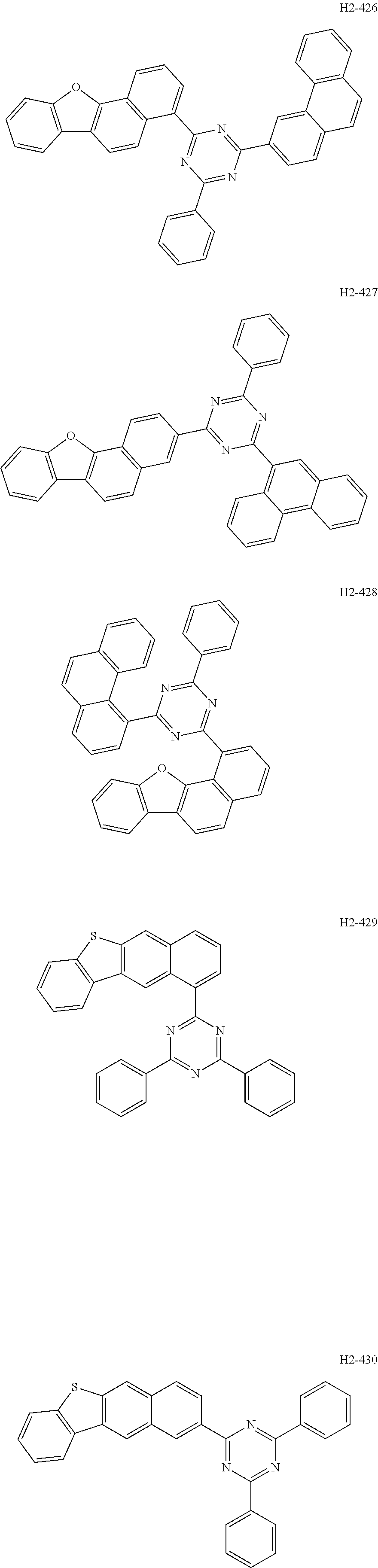 [SMILES: c1ccc(-c2nc(-c3ccc4ccc5ccccc5c4c3)nc(-c3cccc4c3ccc3c5ccccc5oc43)n2)cc1 ; c1ccc(-c2nc(-c3ccc4c(ccc5c6ccccc6oc45)c3)nc(-c3cc4ccccc4c4ccccc34)n2)cc1 ; c1ccc(-c2nc(-c3cccc4ccc5ccccc5c34)nc(-c3cccc4ccc5c6ccccc6oc5c34)n2)cc1 ; c1ccc(-c2nc(-c3ccccc3)nc(-c3cccc4cc5sc6ccccc6c5cc34)n2)cc1 ; c1ccc(-c2nc(-c3ccccc3)nc(-c3ccc4cc5sc6ccccc6c5cc4c3)n2)cc1]

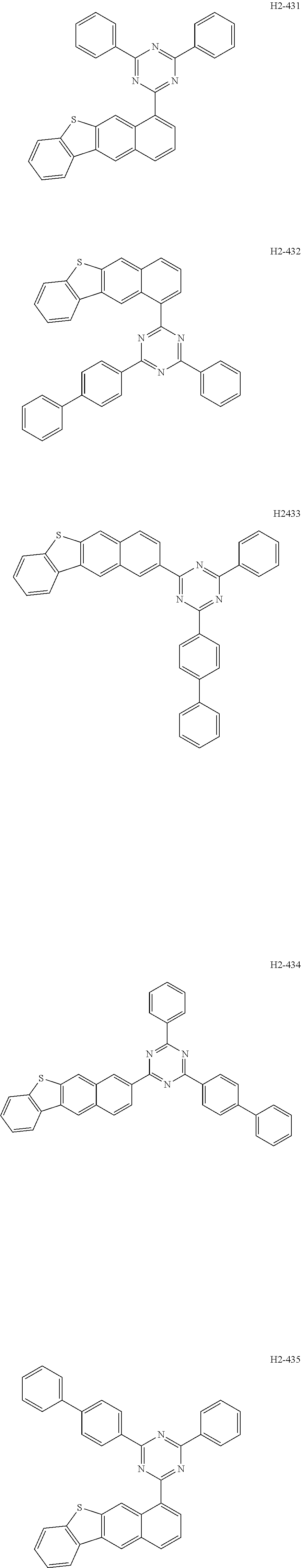 [SMILES: c1ccc(-c2nc(-c3ccccc3)nc(-c3cccc4cc5c(cc34)sc3ccccc35)n2)cc1 ; c1ccc(-c2ccc(-c3nc(-c4ccccc4)nc(-c4cccc5cc6sc7ccccc7c6cc45)n3)cc2)cc1 ; c1ccc(-c2ccc(-c3nc(-c4ccccc4)nc(-c4ccc5cc6sc7ccccc7c6cc5c4)n3)cc2)cc1 ; c1ccc(-c2ccc(-c3nc(-c4ccccc4)nc(-c4ccc5cc6c(cc5c4)sc4ccccc46)n3)cc2)cc1 ; c1ccc(-c2ccc(-c3nc(-c4ccccc4)nc(-c4cccc5cc6c(cc45)sc4ccccc46)n3)cc2)cc1]

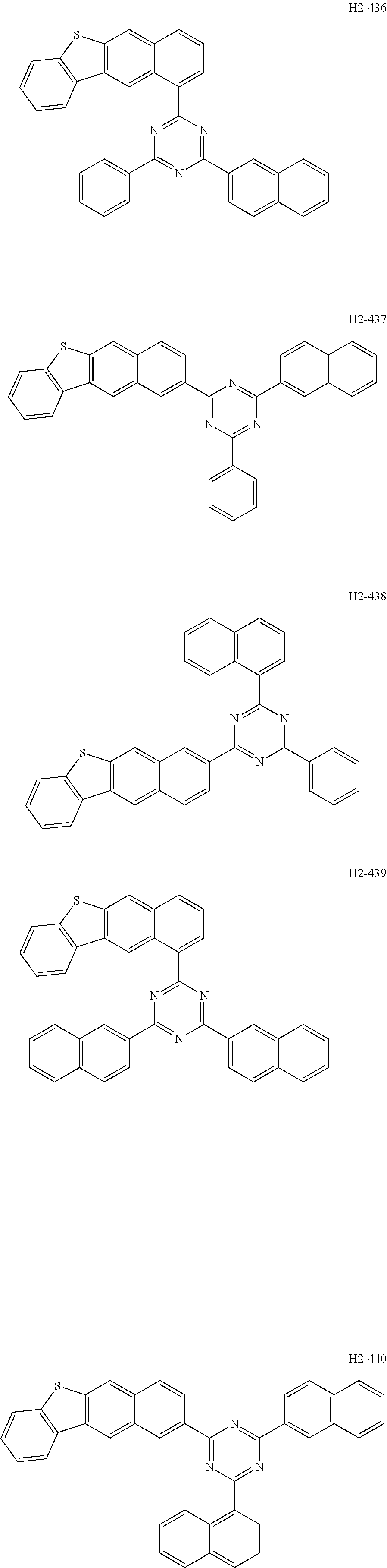 [SMILES: c1ccc(-c2nc(-c3ccc4ccccc4c3)nc(-c3cccc4cc5sc6ccccc6c5cc34)n2)cc1 ; c1ccc(-c2nc(-c3ccc4ccccc4c3)nc(-c3ccc4cc5sc6ccccc6c5cc4c3)n2)cc1 ; c1ccc(-c2nc(-c3ccc4cc5c(cc4c3)sc3ccccc35)nc(-c3cccc4ccccc34)n2)cc1 ; c1ccc2cc(-c3nc(-c4ccc5ccccc5c4)nc(-c4cccc5cc6sc7ccccc7c6cc45)n3)ccc2c1 ; c1ccc2cc(-c3nc(-c4ccc5cc6sc7ccccc7c6cc5c4)nc(-c4cccc5ccccc45)n3)ccc2c1]

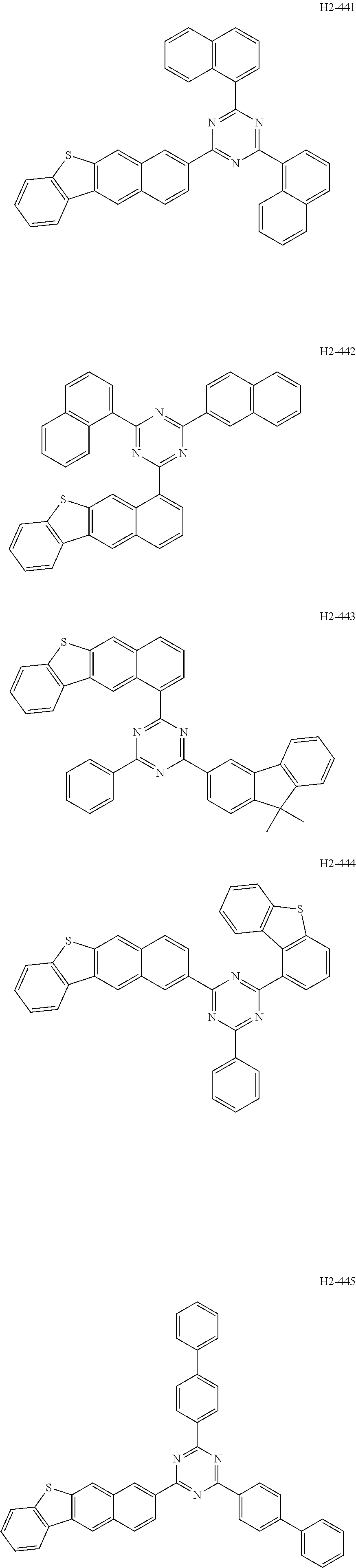 [SMILES: c1ccc2c(-c3nc(-c4ccc5cc6c(cc5c4)sc4ccccc46)nc(-c4cccc5ccccc45)n3)cccc2c1 ; c1ccc2cc(-c3nc(-c4cccc5ccccc45)nc(-c4cccc5cc6c(cc45)sc4ccccc46)n3)ccc2c1 ; CC1(C)c2ccccc2-c2cc(-c3nc(-c4ccccc4)nc(-c4cccc5cc6sc7ccccc7c6cc45)n3)ccc21 ; c1ccc(-c2nc(-c3ccc4cc5sc6ccccc6c5cc4c3)nc(-c3cccc4sc5ccccc5c34)n2)cc1 ; c1ccc(-c2ccc(-c3nc(-c4ccc(-c5ccccc5)cc4)nc(-c4ccc5cc6c(cc5c4)sc4ccccc46)n3)cc2)cc1]

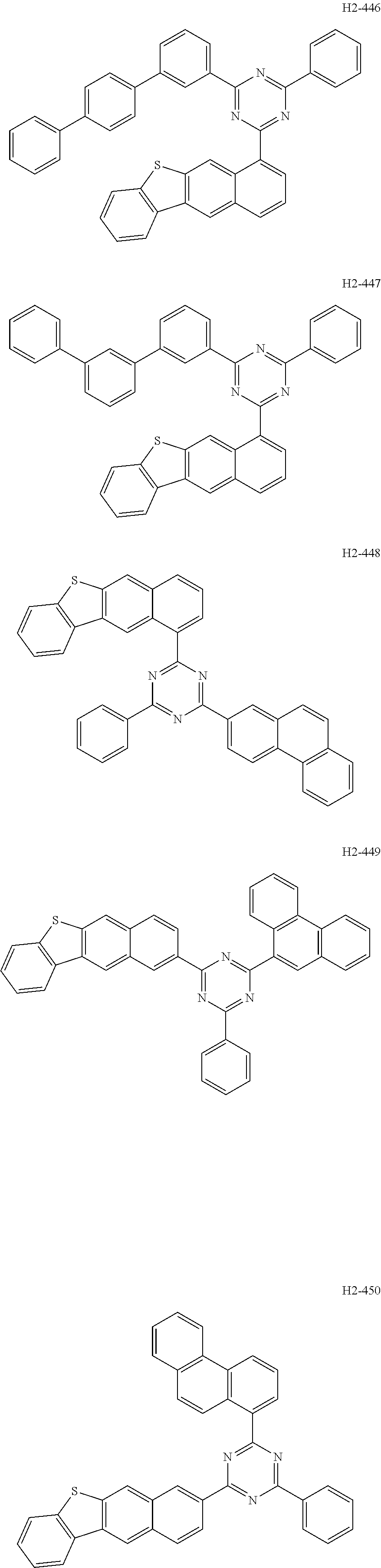 [SMILES: c1ccc(-c2ccc(-c3cccc(-c4nc(-c5ccccc5)nc(-c5cccc6cc7c(cc56)sc5ccccc57)n4)c3)cc2)cc1 ; c1ccc(-c2cccc(-c3cccc(-c4nc(-c5ccccc5)nc(-c5cccc6cc7c(cc56)sc5ccccc57)n4)c3)c2)cc1 ; c1ccc(-c2nc(-c3ccc4c(ccc5ccccc54)c3)nc(-c3cccc4cc5sc6ccccc6c5cc34)n2)cc1 ; c1ccc(-c2nc(-c3ccc4cc5sc6ccccc6c5cc4c3)nc(-c3cc4ccccc4c4ccccc34)n2)cc1 ; c1ccc(-c2nc(-c3ccc4cc5c(cc4c3)sc3ccccc35)nc(-c3cccc4c3ccc3ccccc34)n2)cc1]

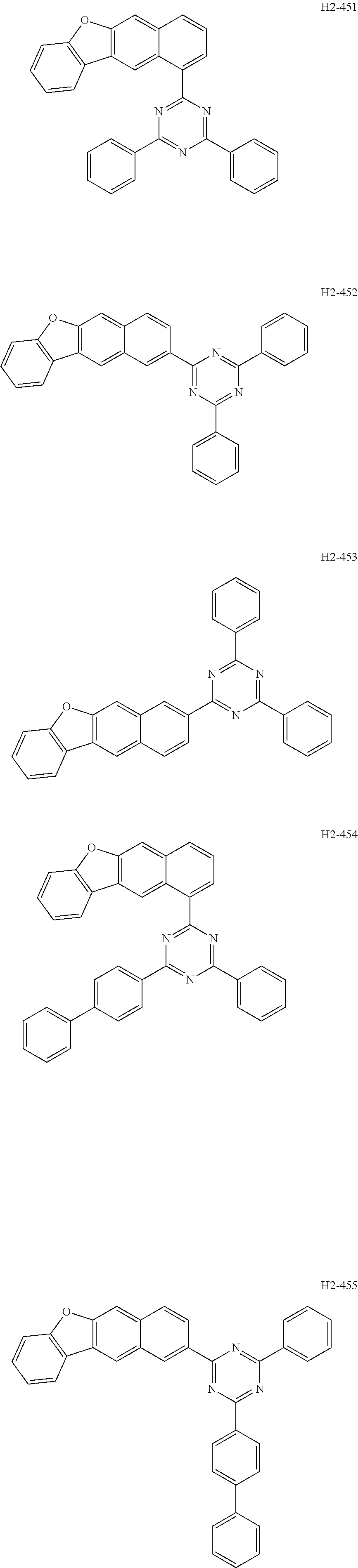 [SMILES: c1ccc(-c2nc(-c3ccccc3)nc(-c3cccc4cc5oc6ccccc6c5cc34)n2)cc1 ; c1ccc(-c2nc(-c3ccccc3)nc(-c3ccc4cc5oc6ccccc6c5cc4c3)n2)cc1 ; c1ccc(-c2nc(-c3ccccc3)nc(-c3ccc4cc5c(cc4c3)oc3ccccc35)n2)cc1 ; c1ccc(-c2ccc(-c3nc(-c4ccccc4)nc(-c4cccc5cc6oc7ccccc7c6cc45)n3)cc2)cc1 ; c1ccc(-c2ccc(-c3nc(-c4ccccc4)nc(-c4ccc5cc6oc7ccccc7c6cc5c4)n3)cc2)cc1]

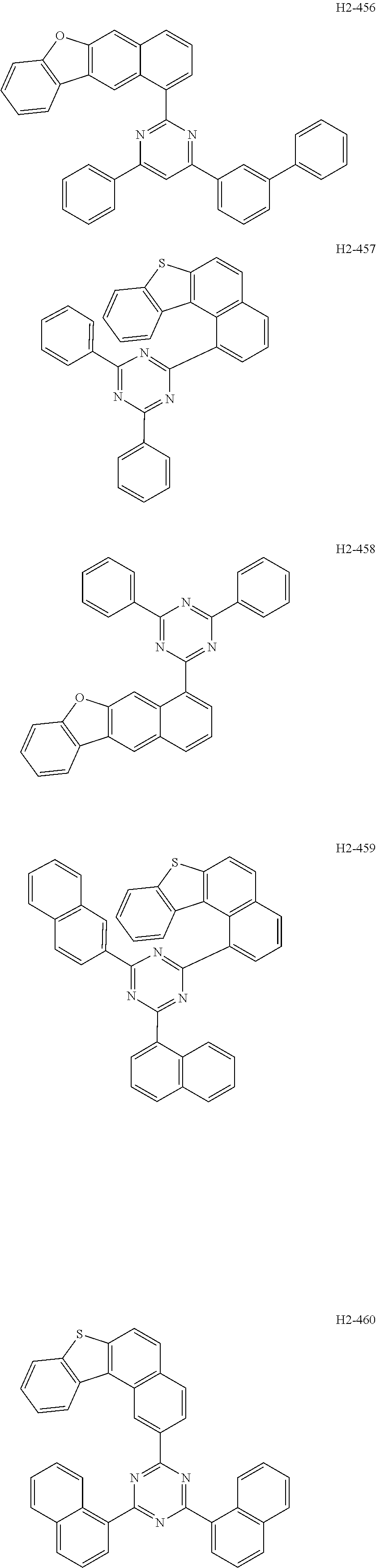 [SMILES: c1ccc(-c2cccc(-c3cc(-c4ccccc4)nc(-c4cccc5cc6oc7ccccc7c6cc45)n3)c2)cc1 ; c1ccc(-c2nc(-c3ccccc3)nc(-c3cccc4ccc5sc6ccccc6c5c34)n2)cc1 ; c1ccc(-c2nc(-c3ccccc3)nc(-c3cccc4cc5c(cc34)oc3ccccc35)n2)cc1 ; c1ccc2cc(-c3nc(-c4cccc5ccccc45)nc(-c4cccc5ccc6sc7ccccc7c6c45)n3)ccc2c1 ; c1ccc2c(-c3nc(-c4ccc5ccc6sc7ccccc7c6c5c4)nc(-c4cccc5ccccc45)n3)cccc2c1]

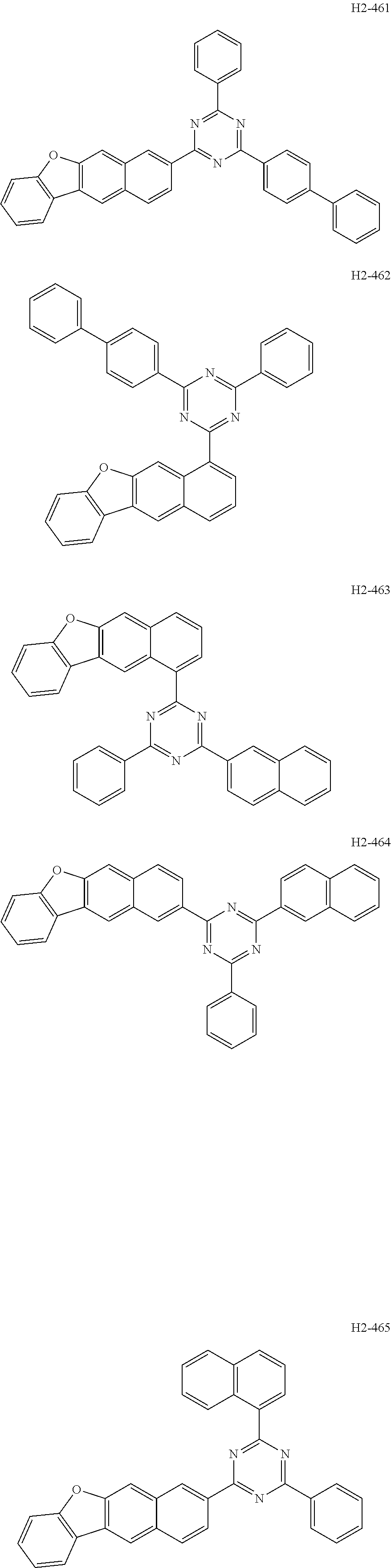 [SMILES: c1ccc(-c2ccc(-c3nc(-c4ccccc4)nc(-c4ccc5cc6c(cc5c4)oc4ccccc46)n3)cc2)cc1 ; c1ccc(-c2ccc(-c3nc(-c4ccccc4)nc(-c4cccc5cc6c(cc45)oc4ccccc46)n3)cc2)cc1 ; c1ccc(-c2nc(-c3ccc4ccccc4c3)nc(-c3cccc4cc5oc6ccccc6c5cc34)n2)cc1 ; c1ccc(-c2nc(-c3ccc4ccccc4c3)nc(-c3ccc4cc5oc6ccccc6c5cc4c3)n2)cc1 ; c1ccc(-c2nc(-c3ccc4cc5c(cc4c3)oc3ccccc35)nc(-c3cccc4ccccc34)n2)cc1]

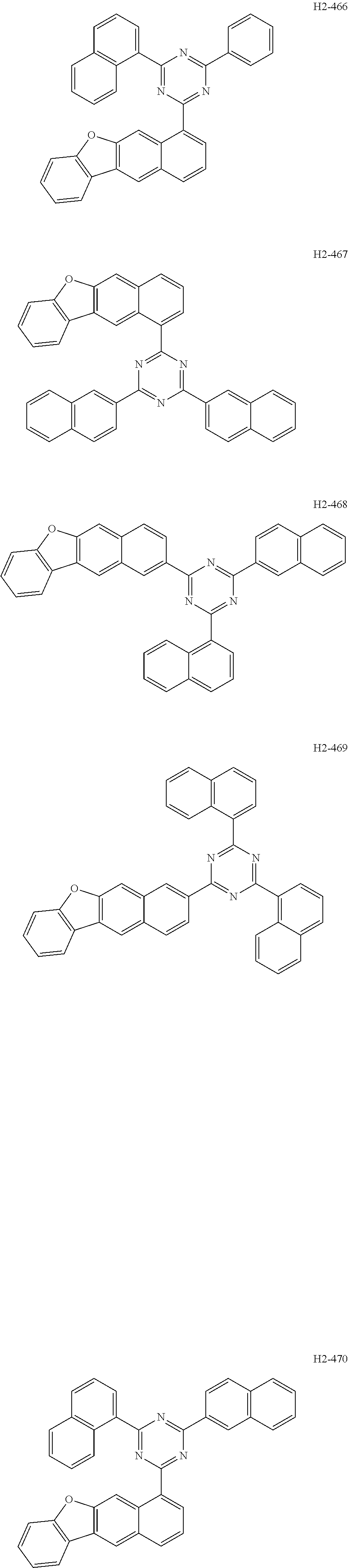 [SMILES: c1ccc(-c2nc(-c3cccc4ccccc34)nc(-c3cccc4cc5c(cc34)oc3ccccc35)n2)cc1 ; c1ccc2cc(-c3nc(-c4ccc5ccccc5c4)nc(-c4cccc5cc6oc7ccccc7c6cc45)n3)ccc2c1 ; c1ccc2cc(-c3nc(-c4ccc5cc6oc7ccccc7c6cc5c4)nc(-c4cccc5ccccc45)n3)ccc2c1 ; c1ccc2c(-c3nc(-c4ccc5cc6c(cc5c4)oc4ccccc46)nc(-c4cccc5ccccc45)n3)cccc2c1 ; c1ccc2cc(-c3nc(-c4cccc5ccccc45)nc(-c4cccc5cc6c(cc45)oc4ccccc46)n3)ccc2c1]

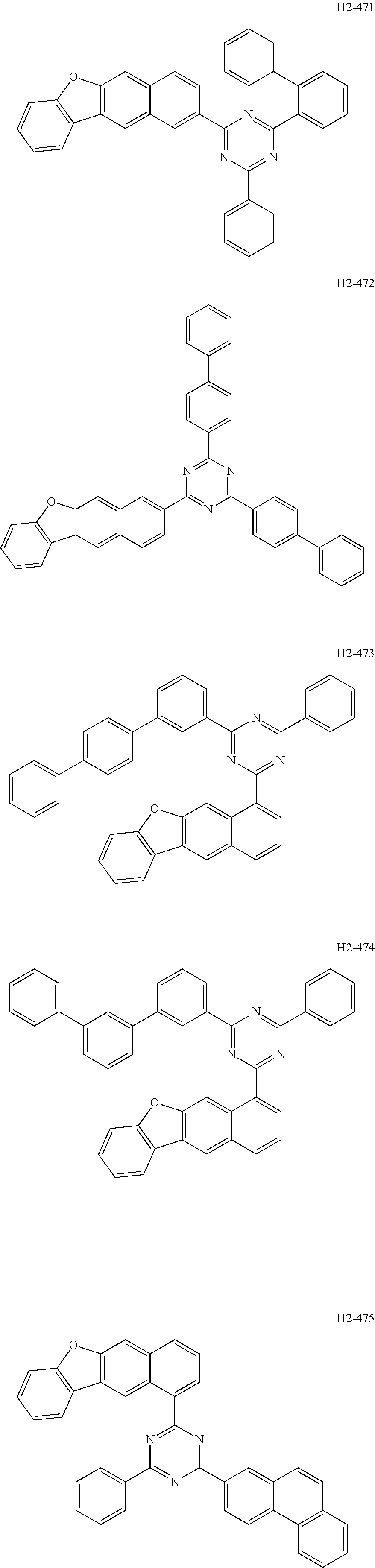 [SMILES: c1ccc(-c2nc(-c3ccc4cc5oc6ccccc6c5cc4c3)nc(-c3ccccc3-c3ccccc3)n2)cc1 ; c1ccc(-c2ccc(-c3nc(-c4ccc(-c5ccccc5)cc4)nc(-c4ccc5cc6c(cc5c4)oc4ccccc46)n3)cc2)cc1 ; c1ccc(-c2ccc(-c3cccc(-c4nc(-c5ccccc5)nc(-c5cccc6cc7c(cc56)oc5ccccc57)n4)c3)cc2)cc1 ; c1ccc(-c2cccc(-c3cccc(-c4nc(-c5ccccc5)nc(-c5cccc6cc7c(cc56)oc5ccccc57)n4)c3)c2)cc1 ; c1ccc(-c2nc(-c3ccc4c(ccc5ccccc54)c3)nc(-c3cccc4cc5oc6ccccc6c5cc34)n2)cc1]

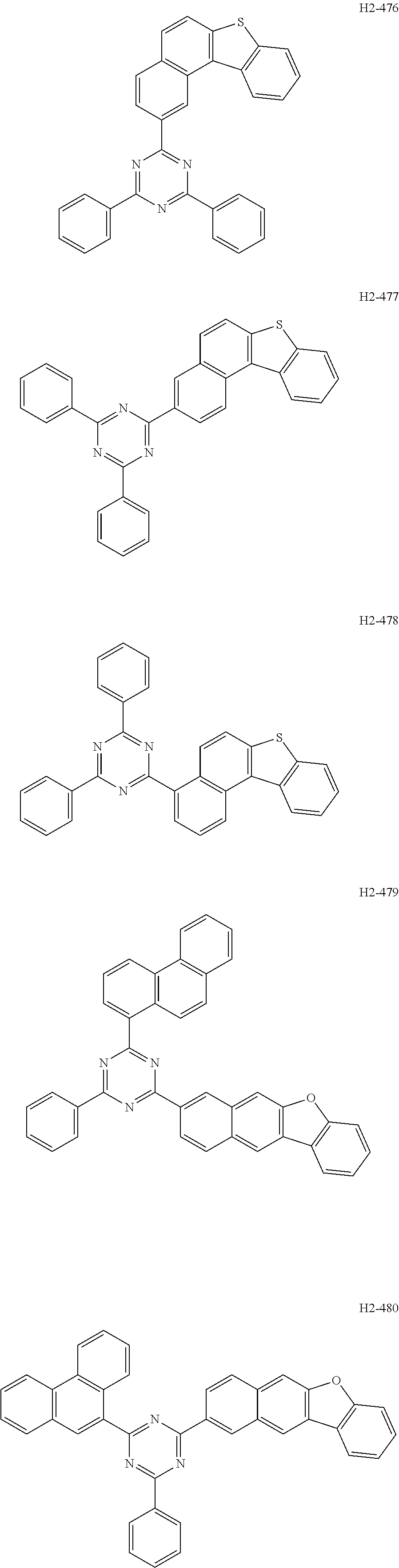 [SMILES: c1ccc(-c2nc(-c3ccccc3)nc(-c3ccc4ccc5sc6ccccc6c5c4c3)n2)cc1 ; c1ccc(-c2nc(-c3ccccc3)nc(-c3ccc4c(ccc5sc6ccccc6c54)c3)n2)cc1 ; c1ccc(-c2nc(-c3ccccc3)nc(-c3cccc4c3ccc3sc5ccccc5c34)n2)cc1 ; c1ccc(-c2nc(-c3ccc4cc5c(cc4c3)oc3ccccc35)nc(-c3cccc4c3ccc3ccccc34)n2)cc1 ; c1ccc(-c2nc(-c3ccc4cc5oc6ccccc6c5cc4c3)nc(-c3cc4ccccc4c4ccccc34)n2)cc1]

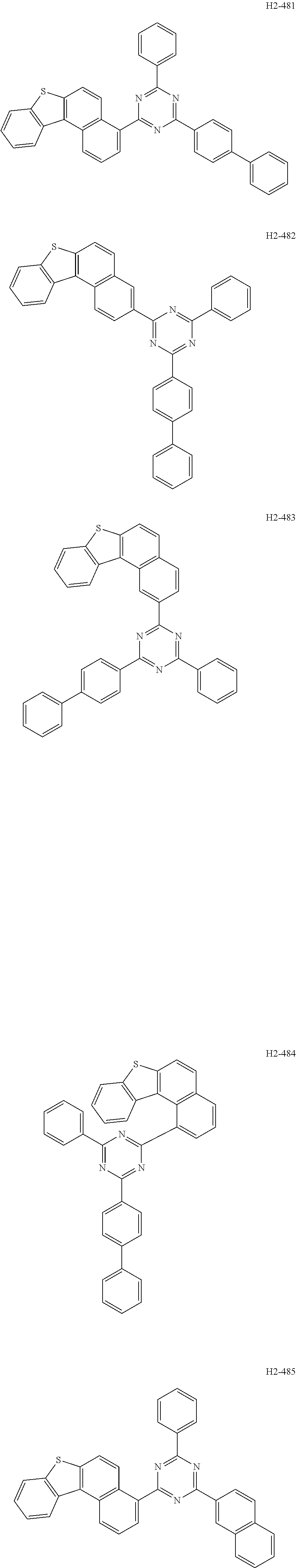 [SMILES: c1ccc(-c2ccc(-c3nc(-c4ccccc4)nc(-c4cccc5c4ccc4sc6ccccc6c45)n3)cc2)cc1 ; c1ccc(-c2ccc(-c3nc(-c4ccccc4)nc(-c4ccc5c(ccc6sc7ccccc7c65)c4)n3)cc2)cc1 ; c1ccc(-c2ccc(-c3nc(-c4ccccc4)nc(-c4ccc5ccc6sc7ccccc7c6c5c4)n3)cc2)cc1 ; c1ccc(-c2ccc(-c3nc(-c4ccccc4)nc(-c4cccc5ccc6sc7ccccc7c6c45)n3)cc2)cc1 ; c1ccc(-c2nc(-c3ccc4ccccc4c3)nc(-c3cccc4c3ccc3sc5ccccc5c34)n2)cc1]

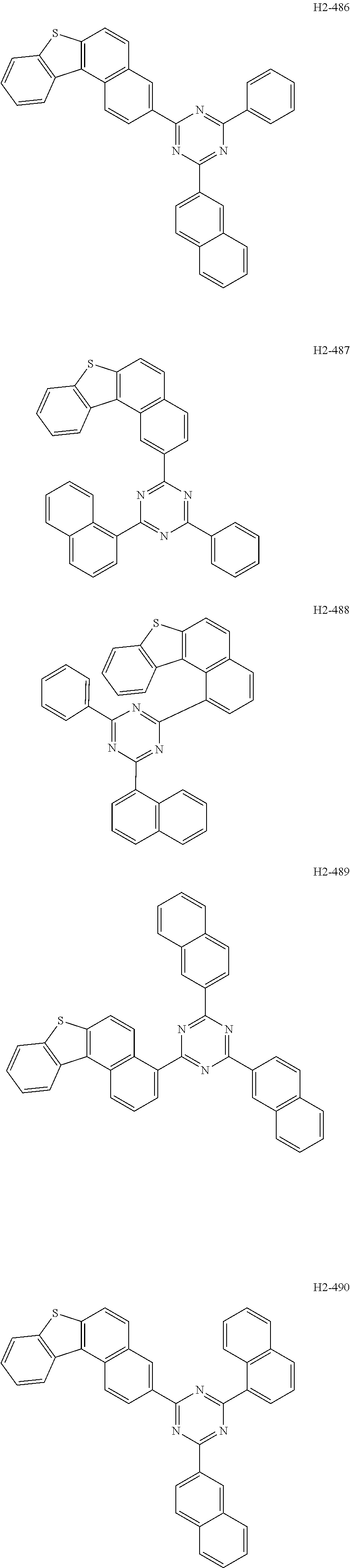 [SMILES: c1ccc(-c2nc(-c3ccc4ccccc4c3)nc(-c3ccc4c(ccc5sc6ccccc6c54)c3)n2)cc1 ; c1ccc(-c2nc(-c3ccc4ccc5sc6ccccc6c5c4c3)nc(-c3cccc4ccccc34)n2)cc1 ; c1ccc(-c2nc(-c3cccc4ccccc34)nc(-c3cccc4ccc5sc6ccccc6c5c34)n2)cc1 ; c1ccc2cc(-c3nc(-c4ccc5ccccc5c4)nc(-c4cccc5c4ccc4sc6ccccc6c45)n3)ccc2c1 ; c1ccc2cc(-c3nc(-c4ccc5c(ccc6sc7ccccc7c65)c4)nc(-c4cccc5ccccc45)n3)ccc2c1]

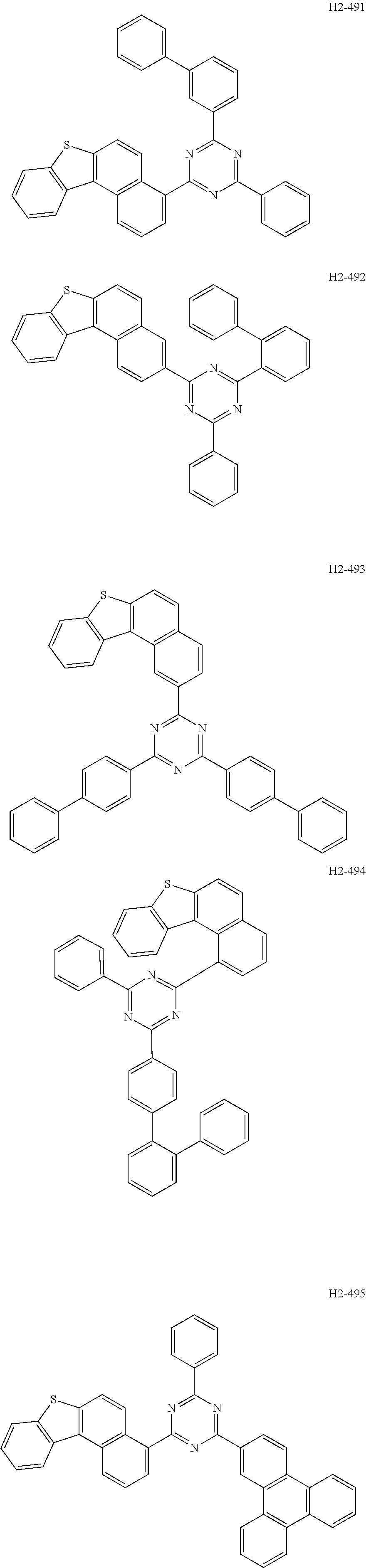 [SMILES: c1ccc(-c2cccc(-c3nc(-c4ccccc4)nc(-c4cccc5c4ccc4sc6ccccc6c45)n3)c2)cc1 ; c1ccc(-c2nc(-c3ccc4c(ccc5sc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6sc7ccccc7c6c5c4)n3)cc2)cc1 ; c1ccc(-c2nc(-c3ccc(-c4ccccc4-c4ccccc4)cc3)nc(-c3cccc4ccc5sc6ccccc6c5c34)n2)cc1 ; c1ccc(-c2nc(-c3ccc4c5ccccc5c5ccccc5c4c3)nc(-c3cccc4c3ccc3sc5ccccc5c34)n2)cc1]

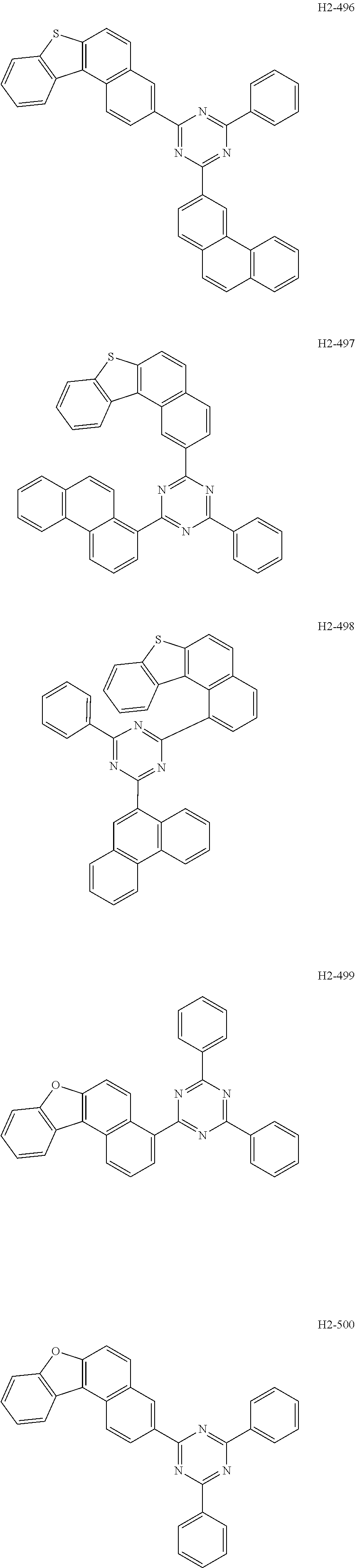 [SMILES: c1ccc(-c2nc(-c3ccc4c(ccc5sc6ccccc6c54)c3)nc(-c3ccc4ccc5ccccc5c4c3)n2)cc1 ; c1ccc(-c2nc(-c3ccc4ccc5sc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5sc6ccccc6c5c34)n2)cc1 ; c1ccc(-c2nc(-c3ccccc3)nc(-c3cccc4c3ccc3oc5ccccc5c34)n2)cc1 ; c1ccc(-c2nc(-c3ccccc3)nc(-c3ccc4c(ccc5oc6ccccc6c54)c3)n2)cc1]

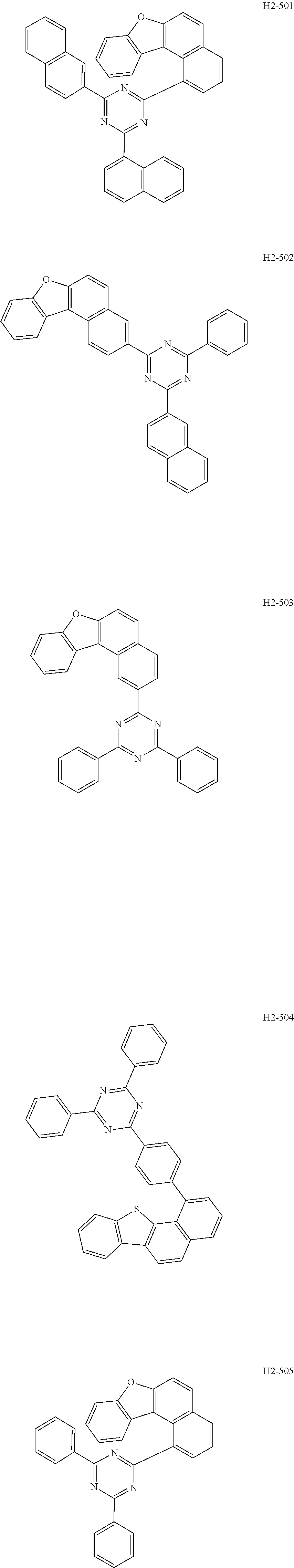 [SMILES: c1ccc2cc(-c3nc(-c4cccc5ccccc45)nc(-c4cccc5ccc6oc7ccccc7c6c45)n3)ccc2c1 ; c1ccc(-c2nc(-c3ccc4ccccc4c3)nc(-c3ccc4c(ccc5oc6ccccc6c54)c3)n2)cc1 ; c1ccc(-c2nc(-c3ccccc3)nc(-c3ccc4ccc5oc6ccccc6c5c4c3)n2)cc1 ; c1ccc(-c2nc(-c3ccccc3)nc(-c3ccc(-c4cccc5ccc6c7ccccc7sc6c45)cc3)n2)cc1 ; c1ccc(-c2nc(-c3ccccc3)nc(-c3cccc4ccc5oc6ccccc6c5c34)n2)cc1]

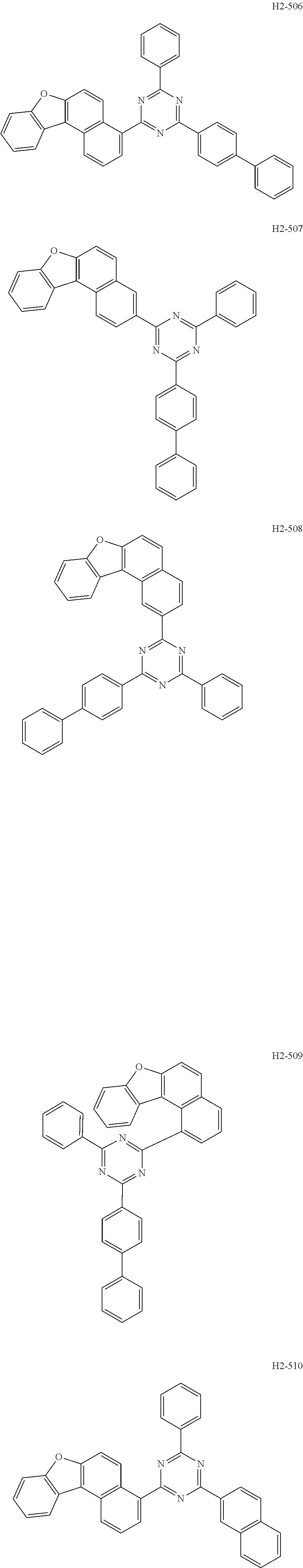 [SMILES: c1ccc(-c2ccc(-c3nc(-c4ccccc4)nc(-c4cccc5c4ccc4oc6ccccc6c45)n3)cc2)cc1 ; c1ccc(-c2ccc(-c3nc(-c4ccccc4)nc(-c4ccc5c(ccc6oc7ccccc7c65)c4)n3)cc2)cc1 ; c1ccc(-c2ccc(-c3nc(-c4ccccc4)nc(-c4ccc5ccc6oc7ccccc7c6c5c4)n3)cc2)cc1 ; c1ccc(-c2ccc(-c3nc(-c4ccccc4)nc(-c4cccc5ccc6oc7ccccc7c6c45)n3)cc2)cc1 ; c1ccc(-c2nc(-c3ccc4ccccc4c3)nc(-c3cccc4c3ccc3oc5ccccc5c34)n2)cc1]

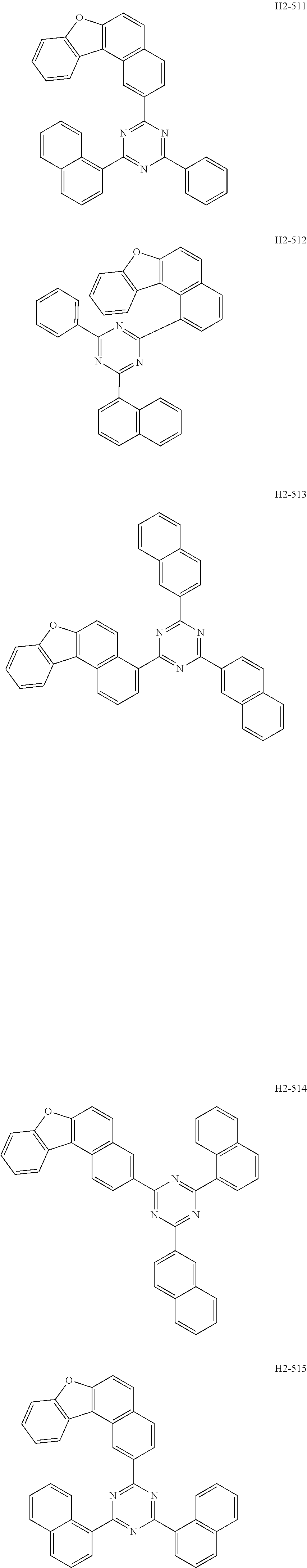 [SMILES: c1ccc(-c2nc(-c3ccc4ccc5oc6ccccc6c5c4c3)nc(-c3cccc4ccccc34)n2)cc1 ; c1ccc(-c2nc(-c3cccc4ccccc34)nc(-c3cccc4ccc5oc6ccccc6c5c34)n2)cc1 ; c1ccc2cc(-c3nc(-c4ccc5ccccc5c4)nc(-c4cccc5c4ccc4oc6ccccc6c45)n3)ccc2c1 ; c1ccc2cc(-c3nc(-c4ccc5c(ccc6oc7ccccc7c65)c4)nc(-c4cccc5ccccc45)n3)ccc2c1 ; c1ccc2c(-c3nc(-c4ccc5ccc6oc7ccccc7c6c5c4)nc(-c4cccc5ccccc45)n3)cccc2c1]

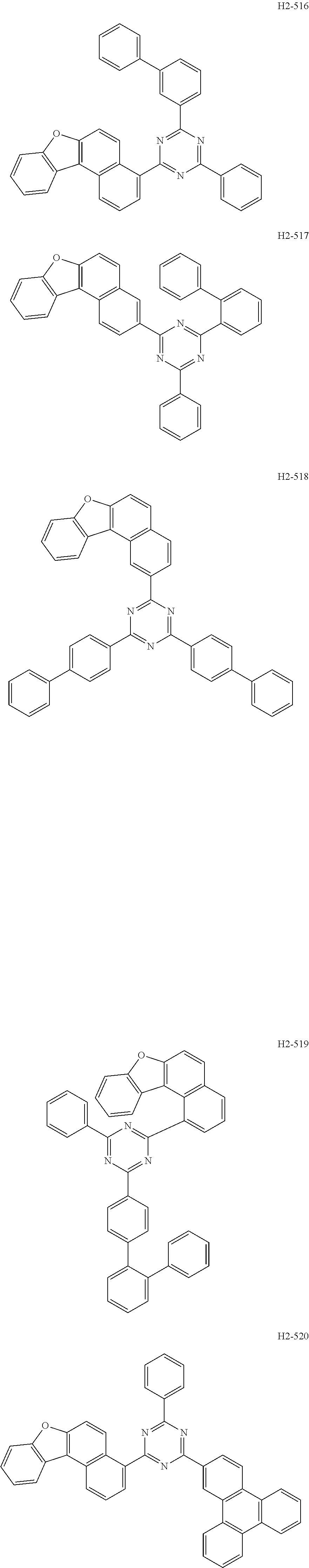 [SMILES: c1ccc(-c2cccc(-c3nc(-c4ccccc4)nc(-c4cccc5c4ccc4oc6ccccc6c45)n3)c2)cc1 ; c1ccc(-c2nc(-c3ccc4c(ccc5oc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6oc7ccccc7c6c5c4)n3)cc2)cc1 ; c1ccc(-c2nc(-c3ccc(-c4ccccc4-c4ccccc4)cc3)nc(-c3cccc4ccc5oc6ccccc6c5c34)n2)cc1 ; c1ccc(-c2nc(-c3ccc4c5ccccc5c5ccccc5c4c3)nc(-c3cccc4c3ccc3oc5ccccc5c34)n2)cc1]

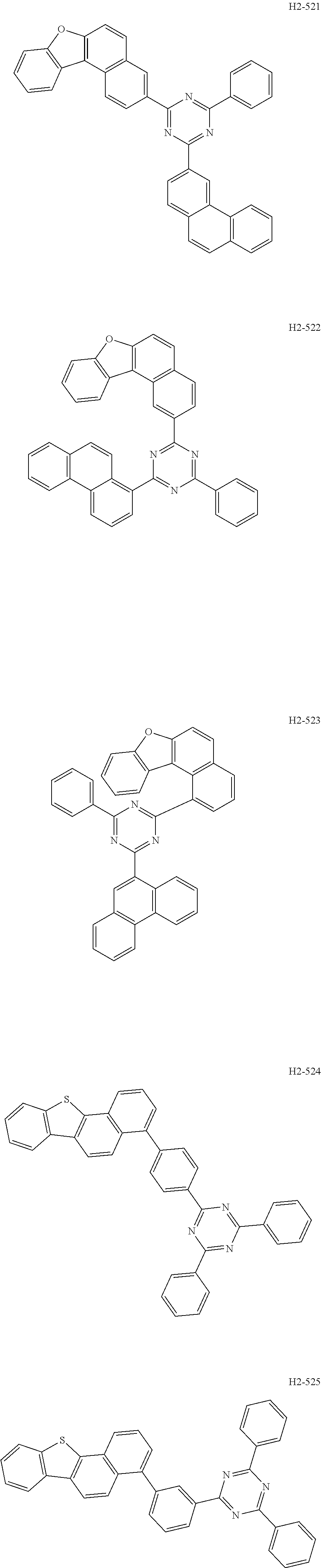 [SMILES: c1ccc(-c2nc(-c3ccc4c(ccc5oc6ccccc6c54)c3)nc(-c3ccc4ccc5ccccc5c4c3)n2)cc1 ; c1ccc(-c2nc(-c3ccc4ccc5oc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5oc6ccccc6c5c34)n2)cc1 ; c1ccc(-c2nc(-c3ccccc3)nc(-c3ccc(-c4cccc5c4ccc4c6ccccc6sc54)cc3)n2)cc1 ; c1ccc(-c2nc(-c3ccccc3)nc(-c3cccc(-c4cccc5c4ccc4c6ccccc6sc54)c3)n2)cc1]

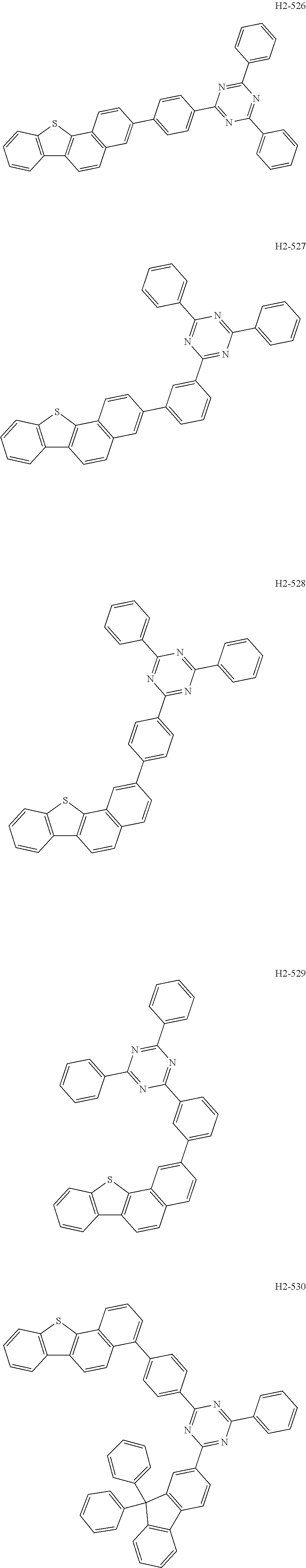 [SMILES: c1ccc(-c2nc(-c3ccccc3)nc(-c3ccc(-c4ccc5c(ccc6c7ccccc7sc56)c4)cc3)n2)cc1 ; c1ccc(-c2nc(-c3ccccc3)nc(-c3cccc(-c4ccc5c(ccc6c7ccccc7sc56)c4)c3)n2)cc1 ; c1ccc(-c2nc(-c3ccccc3)nc(-c3ccc(-c4ccc5ccc6c7ccccc7sc6c5c4)cc3)n2)cc1 ; c1ccc(-c2nc(-c3ccccc3)nc(-c3cccc(-c4ccc5ccc6c7ccccc7sc6c5c4)c3)n2)cc1 ; c1ccc(-c2nc(-c3ccc(-c4cccc5c4ccc4c6ccccc6sc54)cc3)nc(-c3ccc4c(c3)C(c3ccccc3)(c3ccccc3)c3ccccc3-4)n2)cc1]

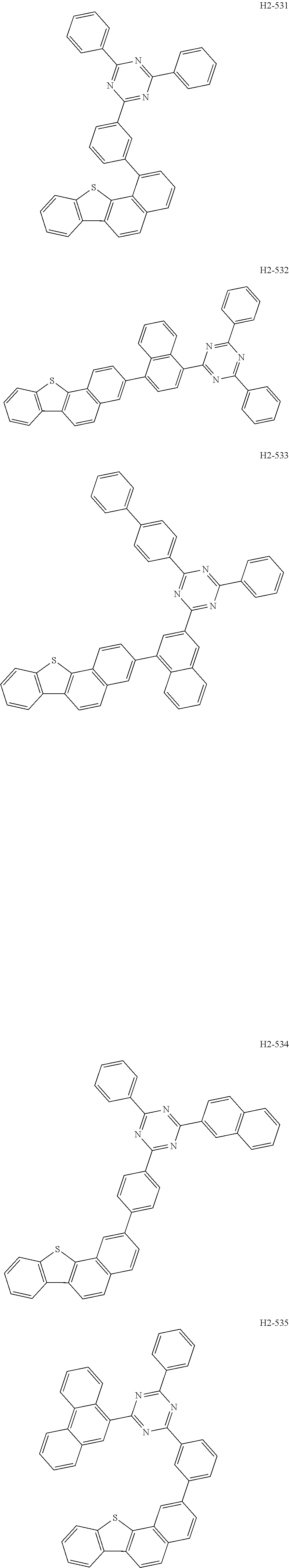 [SMILES: c1ccc(-c2nc(-c3ccccc3)nc(-c3cccc(-c4cccc5ccc6c7ccccc7sc6c45)c3)n2)cc1 ; c1ccc(-c2nc(-c3ccccc3)nc(-c3ccc(-c4ccc5c(ccc6c7ccccc7sc56)c4)c4ccccc34)n2)cc1 ; c1ccc(-c2ccc(-c3nc(-c4ccccc4)nc(-c4cc(-c5ccc6c(ccc7c8ccccc8sc67)c5)c5ccccc5c4)n3)cc2)cc1 ; c1ccc(-c2nc(-c3ccc(-c4ccc5ccc6c7ccccc7sc6c5c4)cc3)nc(-c3ccc4ccccc4c3)n2)cc1 ; c1ccc(-c2nc(-c3cccc(-c4ccc5ccc6c7ccccc7sc6c5c4)c3)nc(-c3cc4ccccc4c4ccccc34)n2)cc1]

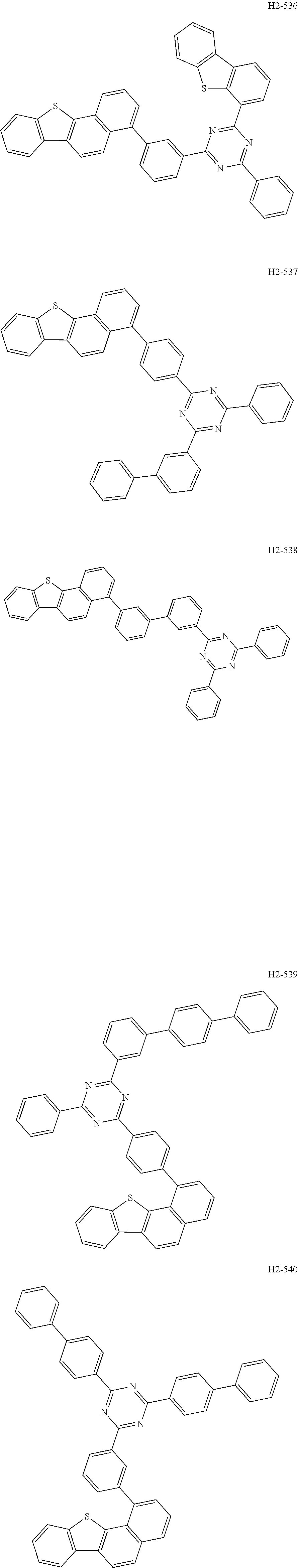 [SMILES: c1ccc(-c2nc(-c3cccc(-c4cccc5c4ccc4c6ccccc6sc54)c3)nc(-c3cccc4c3sc3ccccc34)n2)cc1 ; c1ccc(-c2cccc(-c3nc(-c4ccccc4)nc(-c4ccc(-c5cccc6c5ccc5c7ccccc7sc65)cc4)n3)c2)cc1 ; c1ccc(-c2nc(-c3ccccc3)nc(-c3cccc(-c4cccc(-c5cccc6c5ccc5c7ccccc7sc65)c4)c3)n2)cc1 ; c1ccc(-c2ccc(-c3cccc(-c4nc(-c5ccccc5)nc(-c5ccc(-c6cccc7ccc8c9ccccc9sc8c67)cc5)n4)c3)cc2)cc1 ; c1ccc(-c2ccc(-c3nc(-c4ccc(-c5ccccc5)cc4)nc(-c4cccc(-c5cccc6ccc7c8ccccc8sc7c56)c4)n3)cc2)cc1]

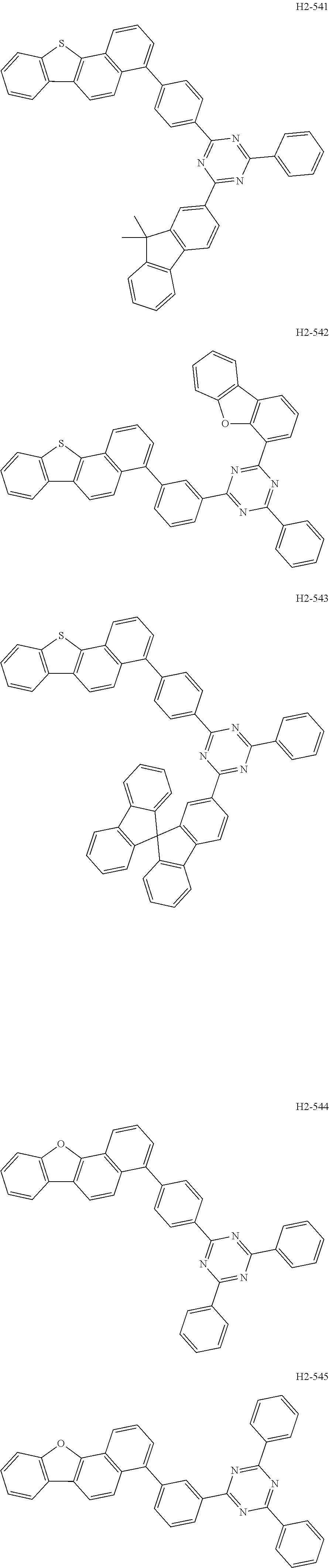 [SMILES: CC1(C)c2ccccc2-c2ccc(-c3nc(-c4ccccc4)nc(-c4ccc(-c5cccc6c5ccc5c7ccccc7sc65)cc4)n3)cc21 ; c1ccc(-c2nc(-c3cccc(-c4cccc5c4ccc4c6ccccc6sc54)c3)nc(-c3cccc4c3oc3ccccc34)n2)cc1 ; c1ccc(-c2nc(-c3ccc(-c4cccc5c4ccc4c6ccccc6sc54)cc3)nc(-c3ccc4c(c3)C3(c5ccccc5-c5ccccc53)c3ccccc3-4)n2)cc1 ; c1ccc(-c2nc(-c3ccccc3)nc(-c3ccc(-c4cccc5c4ccc4c6ccccc6oc54)cc3)n2)cc1 ; c1ccc(-c2nc(-c3ccccc3)nc(-c3cccc(-c4cccc5c4ccc4c6ccccc6oc54)c3)n2)cc1]

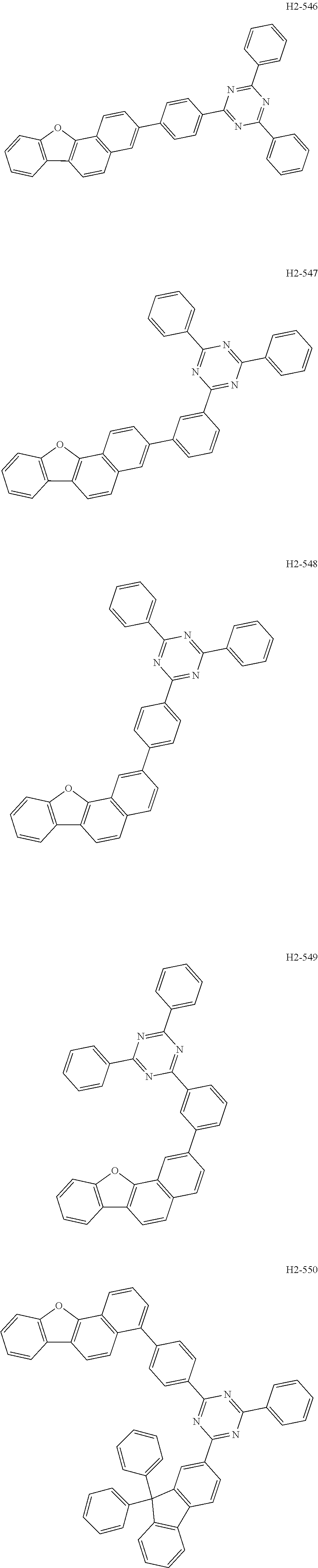 [SMILES: c1ccc(-c2nc(-c3ccccc3)nc(-c3ccc(-c4ccc5c(ccc6c7ccccc7oc56)c4)cc3)n2)cc1 ; c1ccc(-c2nc(-c3ccccc3)nc(-c3cccc(-c4ccc5c(ccc6c7ccccc7oc56)c4)c3)n2)cc1 ; c1ccc(-c2nc(-c3ccccc3)nc(-c3ccc(-c4ccc5ccc6c7ccccc7oc6c5c4)cc3)n2)cc1 ; c1ccc(-c2nc(-c3ccccc3)nc(-c3cccc(-c4ccc5ccc6c7ccccc7oc6c5c4)c3)n2)cc1 ; c1ccc(-c2nc(-c3ccc(-c4cccc5c4ccc4c6ccccc6oc54)cc3)nc(-c3ccc4c(c3)C(c3ccccc3)(c3ccccc3)c3ccccc3-4)n2)cc1]

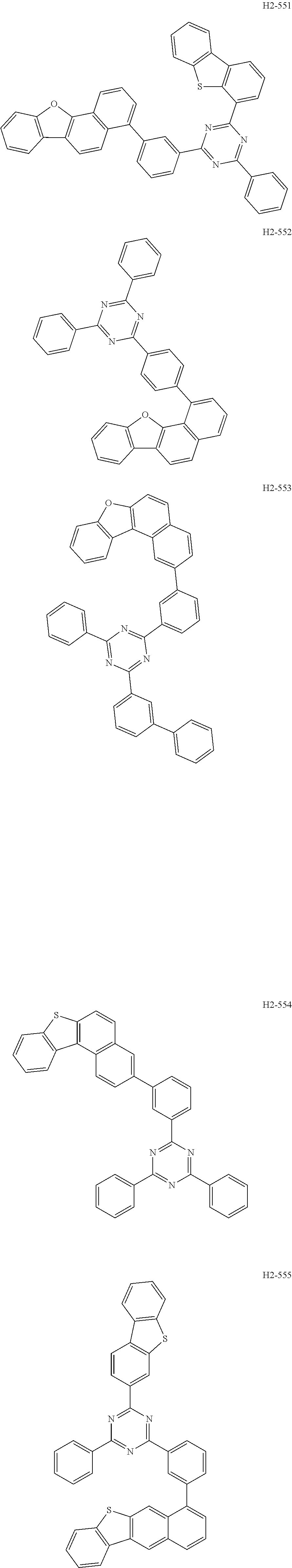 [SMILES: c1ccc(-c2nc(-c3cccc(-c4cccc5c4ccc4c6ccccc6oc54)c3)nc(-c3cccc4c3sc3ccccc34)n2)cc1 ; c1ccc(-c2nc(-c3ccccc3)nc(-c3ccc(-c4cccc5ccc6c7ccccc7oc6c45)cc3)n2)cc1 ; c1ccc(-c2cccc(-c3nc(-c4ccccc4)nc(-c4cccc(-c5ccc6ccc7oc8ccccc8c7c6c5)c4)n3)c2)cc1 ; c1ccc(-c2nc(-c3ccccc3)nc(-c3cccc(-c4ccc5c(ccc6sc7ccccc7c65)c4)c3)n2)cc1 ; c1ccc(-c2nc(-c3cccc(-c4cccc5cc6c(cc45)sc4ccccc46)c3)nc(-c3ccc4c(c3)sc3ccccc34)n2)cc1]

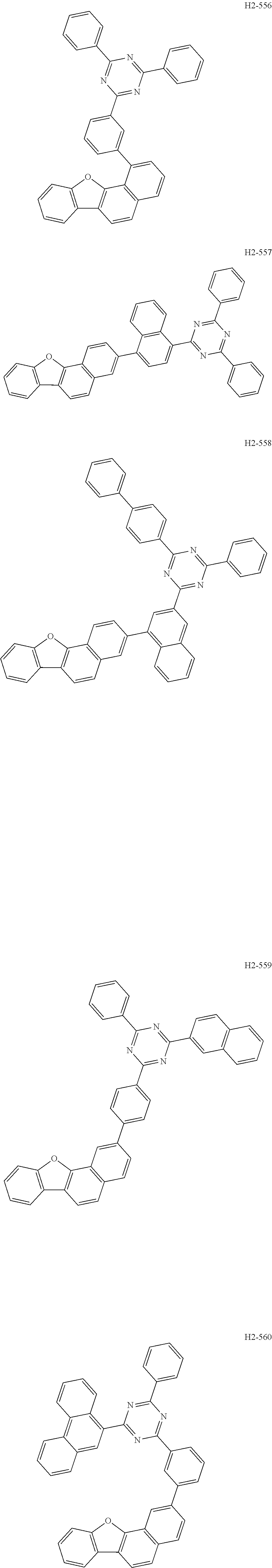 [SMILES: c1ccc(-c2nc(-c3ccccc3)nc(-c3cccc(-c4cccc5ccc6c7ccccc7oc6c45)c3)n2)cc1 ; c1ccc(-c2nc(-c3ccccc3)nc(-c3ccc(-c4ccc5c(ccc6c7ccccc7oc56)c4)c4ccccc34)n2)cc1 ; c1ccc(-c2ccc(-c3nc(-c4ccccc4)nc(-c4cc(-c5ccc6c(ccc7c8ccccc8oc67)c5)c5ccccc5c4)n3)cc2)cc1 ; c1ccc(-c2nc(-c3ccc(-c4ccc5ccc6c7ccccc7oc6c5c4)cc3)nc(-c3ccc4ccccc4c3)n2)cc1 ; c1ccc(-c2nc(-c3cccc(-c4ccc5ccc6c7ccccc7oc6c5c4)c3)nc(-c3cc4ccccc4c4ccccc34)n2)cc1]

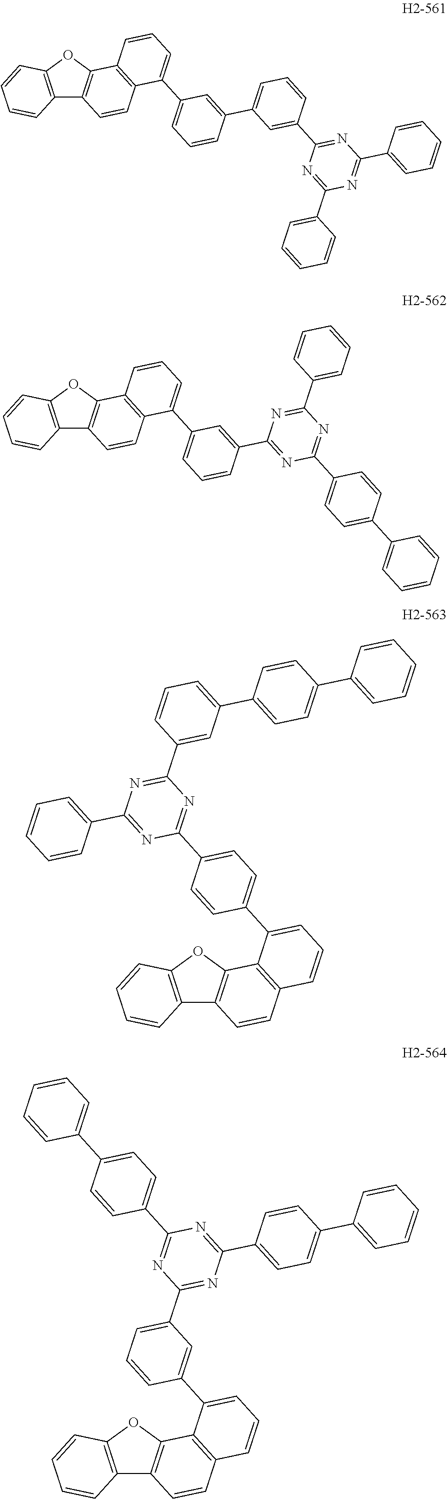 [SMILES: c1ccc(-c2nc(-c3ccccc3)nc(-c3cccc(-c4cccc(-c5cccc6c5ccc5c7ccccc7oc65)c4)c3)n2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2ccc(-c3cccc(-c4nc(-c5ccccc5)nc(-c5ccc(-c6cccc7ccc8c9ccccc9oc8c67)cc5)n4)c3)cc2)cc1 ; c1ccc(-c2ccc(-c3nc(-c4ccc(-c5ccccc5)cc4)nc(-c4cccc(-c5cccc6ccc7c8ccccc8oc7c56)c4)n3)cc2)cc1]

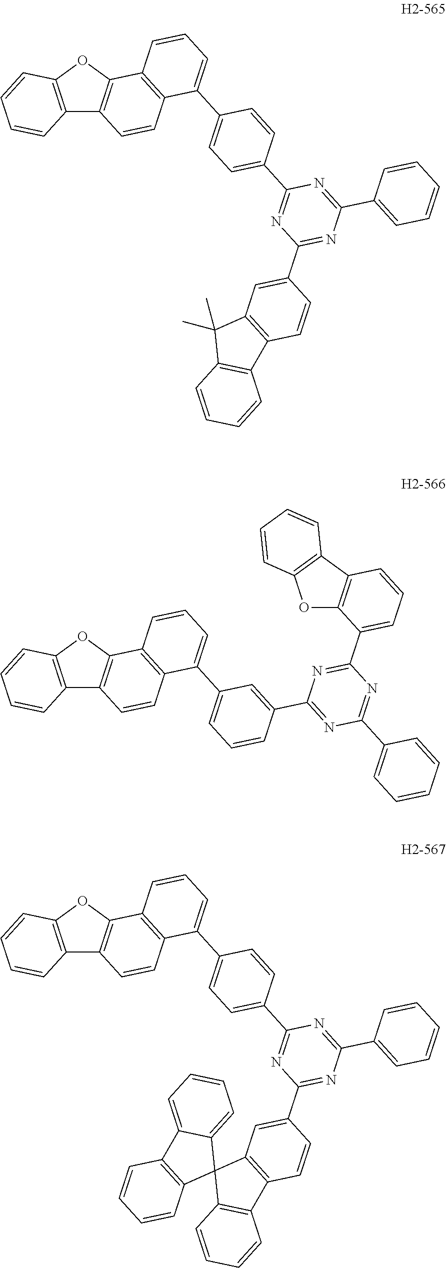 [SMILES: CC1(C)c2ccccc2-c2ccc(-c3nc(-c4ccccc4)nc(-c4ccc(-c5cccc6c5ccc5c7ccccc7oc65)cc4)n3)cc21 ; c1ccc(-c2nc(-c3cccc(-c4cccc5c4ccc4c6ccccc6oc54)c3)nc(-c3cccc4c3oc3ccccc34)n2)cc1 ; c1ccc(-c2nc(-c3ccc(-c4cccc5c4ccc4c6ccccc6oc54)cc3)nc(-c3ccc4c(c3)C3(c5ccccc5-c5ccccc53)c3ccccc3-4)n2)cc1]

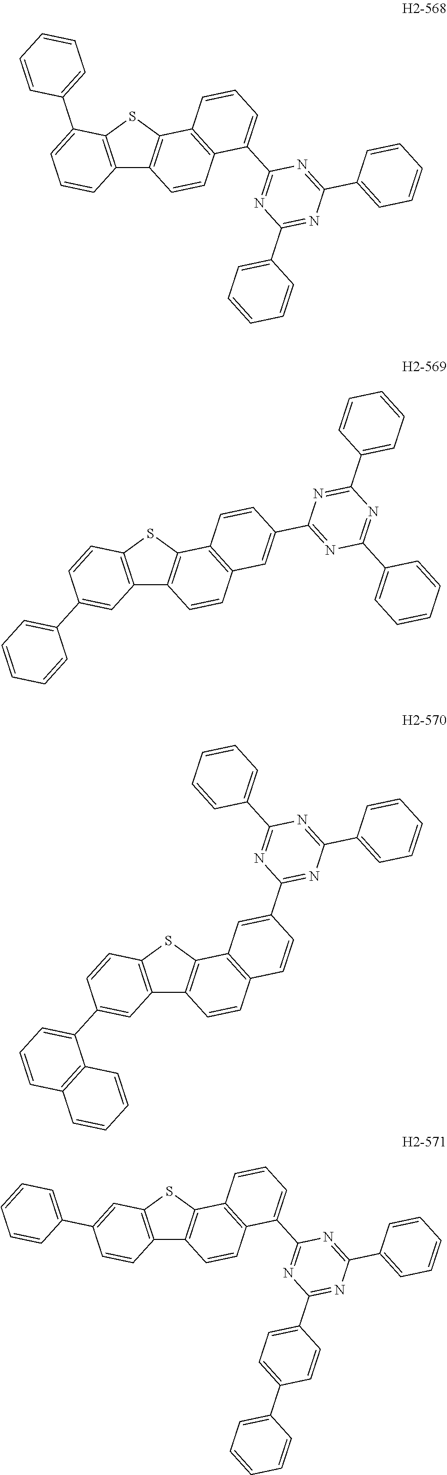 [SMILES: c1ccc(-c2nc(-c3ccccc3)nc(-c3cccc4c3ccc3c5cccc(-c6ccccc6)c5sc43)n2)cc1 ; c1ccc(-c2ccc3sc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6sc5c4c3)n2)cc1 ; c1ccc(-c2ccc(-c3nc(-c4ccccc4)nc(-c4cccc5c4ccc4c6ccc(-c7ccccc7)cc6sc54)n3)cc2)cc1]

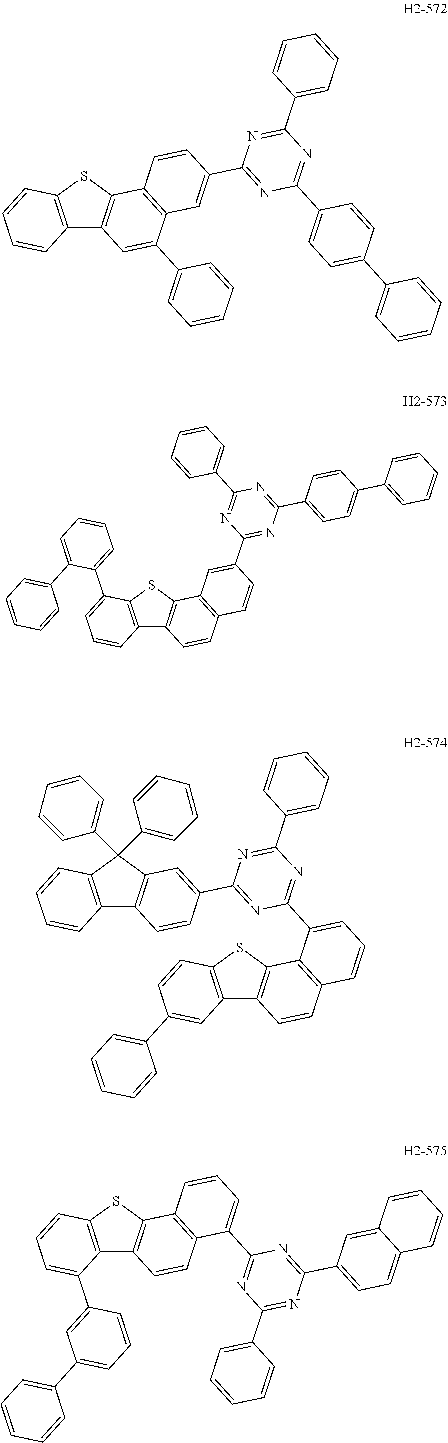 [SMILES: c1ccc(-c2ccc(-c3nc(-c4ccccc4)nc(-c4ccc5c(c4)c(-c4ccccc4)cc4c6ccccc6sc54)n3)cc2)cc1 ; c1ccc(-c2ccc(-c3nc(-c4ccccc4)nc(-c4ccc5ccc6c7cccc(-c8ccccc8-c8ccccc8)c7sc6c5c4)n3)cc2)cc1 ; c1ccc(-c2ccc3sc4c(ccc5cccc(-c6nc(-c7ccccc7)nc(-c7ccc8c(c7)C(c7ccccc7)(c7ccccc7)c7ccccc7-8)n6)c54)c3c2)cc1 ; c1ccc(-c2cccc(-c3cccc4sc5c6cccc(-c7nc(-c8ccccc8)nc(-c8ccc9ccccc9c8)n7)c6ccc5c34)c2)cc1]

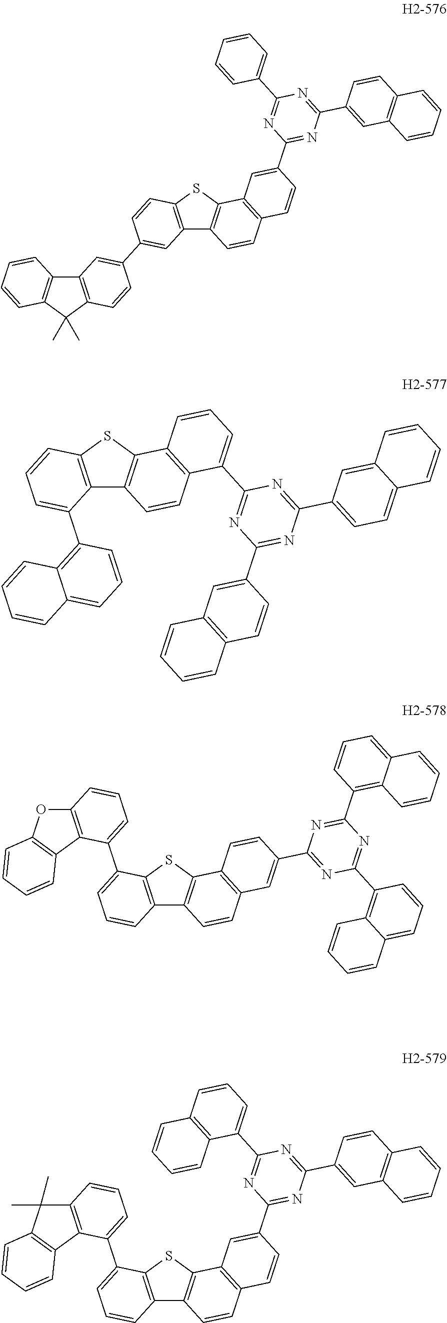 [SMILES: CC1(C)c2ccccc2-c2cc(-c3ccc4sc5c6cc(-c7nc(-c8ccccc8)nc(-c8ccc9ccccc9c8)n7)ccc6ccc5c4c3)ccc21 ; c1ccc2cc(-c3nc(-c4ccc5ccccc5c4)nc(-c4cccc5c4ccc4c5sc5cccc(-c6cccc7ccccc67)c54)n3)ccc2c1 ; c1ccc2c(-c3nc(-c4ccc5c(ccc6c7cccc(-c8cccc9oc%10ccccc%10c89)c7sc56)c4)nc(-c4cccc5ccccc45)n3)cccc2c1 ; CC1(C)c2ccccc2-c2c(-c3cccc4c3sc3c5cc(-c6nc(-c7ccc8ccccc8c7)nc(-c7cccc8ccccc78)n6)ccc5ccc43)cccc21]

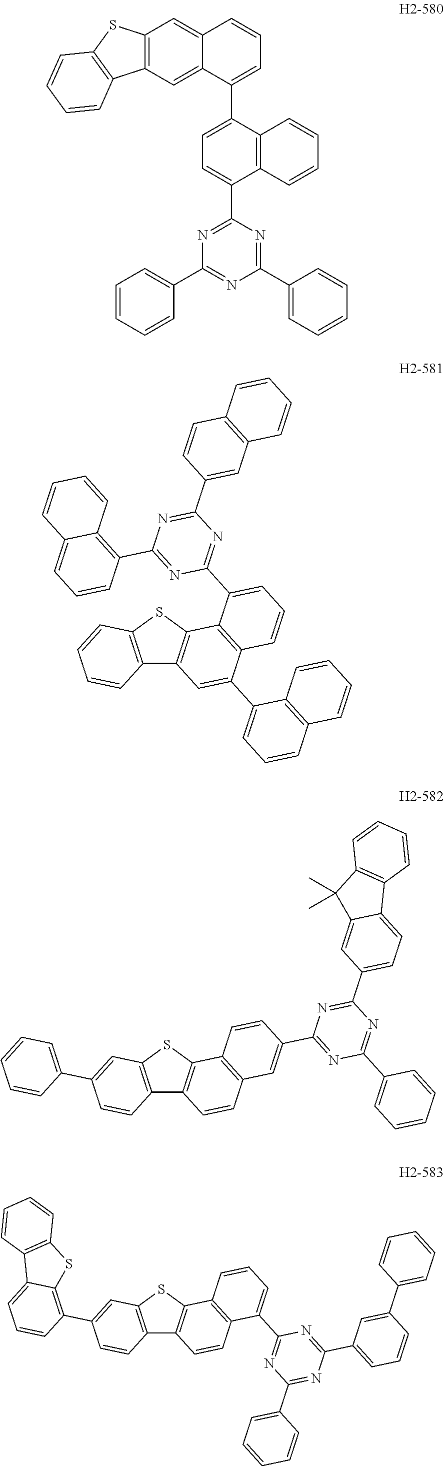 [SMILES: c1ccc(-c2nc(-c3ccccc3)nc(-c3ccc(-c4cccc5cc6sc7ccccc7c6cc45)c4ccccc34)n2)cc1 ; c1ccc2cc(-c3nc(-c4cccc5ccccc45)nc(-c4cccc5c(-c6cccc7ccccc67)cc6c7ccccc7sc6c45)n3)ccc2c1 ; CC1(C)c2ccccc2-c2ccc(-c3nc(-c4ccccc4)nc(-c4ccc5c(ccc6c7ccc(-c8ccccc8)cc7sc56)c4)n3)cc21 ; c1ccc(-c2cccc(-c3nc(-c4ccccc4)nc(-c4cccc5c4ccc4c6ccc(-c7cccc8c7sc7ccccc78)cc6sc54)n3)c2)cc1]

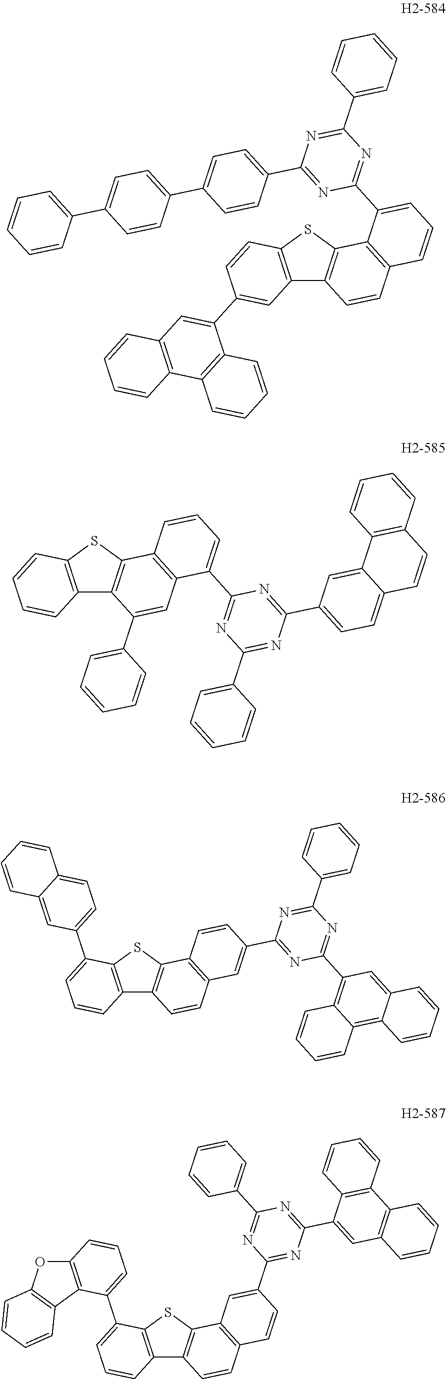 [SMILES: c1ccc(-c2ccc(-c3ccc(-c4nc(-c5ccccc5)nc(-c5cccc6ccc7c8cc(-c9cc%10ccccc%10c%10ccccc9%10)ccc8sc7c56)n4)cc3)cc2)cc1 ; c1ccc(-c2nc(-c3ccc4ccc5ccccc5c4c3)nc(-c3cccc4c3cc(-c3ccccc3)c3c5ccccc5sc43)n2)cc1 ; c1ccc(-c2nc(-c3ccc4c(ccc5c6cccc(-c7ccc8ccccc8c7)c6sc45)c3)nc(-c3cc4ccccc4c4ccccc34)n2)cc1 ; c1ccc(-c2nc(-c3ccc4ccc5c6cccc(-c7cccc8oc9ccccc9c78)c6sc5c4c3)nc(-c3cc4ccccc4c4ccccc34)n2)cc1]

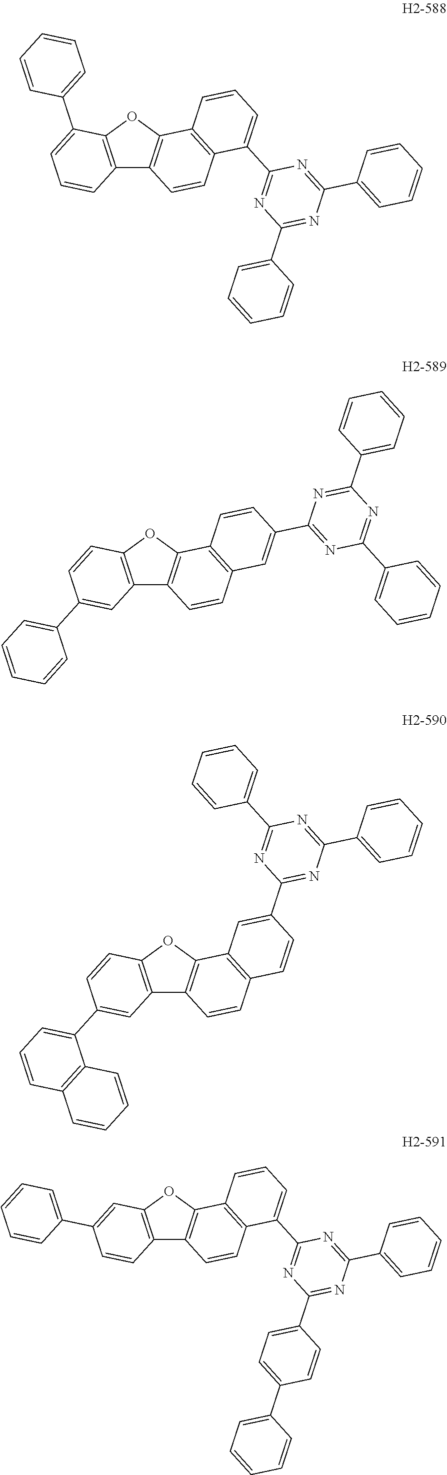 [SMILES: c1ccc(-c2nc(-c3ccccc3)nc(-c3cccc4c3ccc3c5cccc(-c6ccccc6)c5oc43)n2)cc1 ; c1ccc(-c2ccc3oc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6oc5c4c3)n2)cc1 ; c1ccc(-c2ccc(-c3nc(-c4ccccc4)nc(-c4cccc5c4ccc4c6ccc(-c7ccccc7)cc6oc54)n3)cc2)cc1]

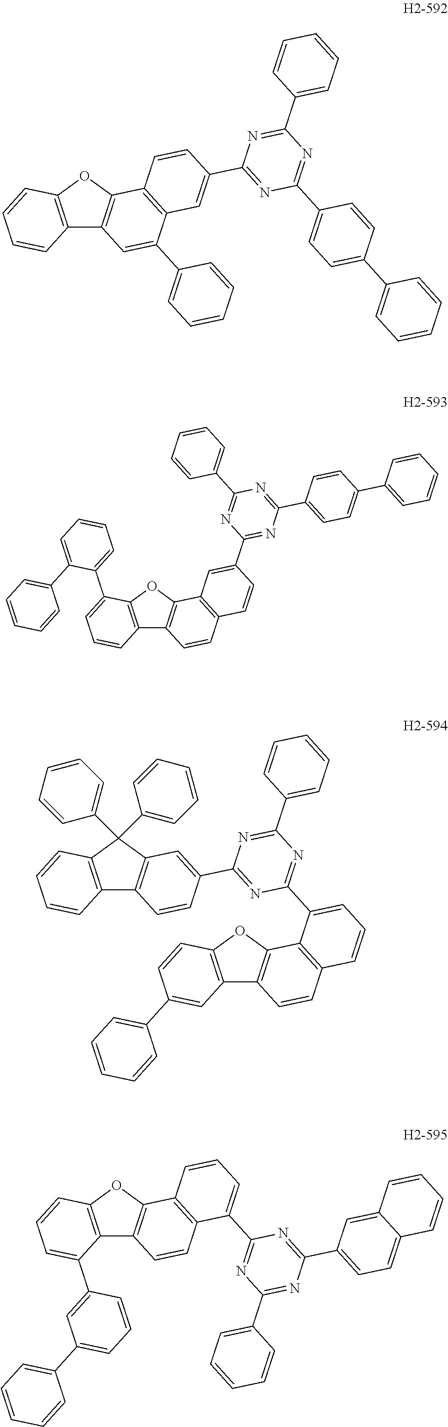 [SMILES: c1ccc(-c2ccc(-c3nc(-c4ccccc4)nc(-c4ccc5c(c4)c(-c4ccccc4)cc4c6ccccc6oc54)n3)cc2)cc1 ; c1ccc(-c2ccc(-c3nc(-c4ccccc4)nc(-c4ccc5ccc6c7cccc(-c8ccccc8-c8ccccc8)c7oc6c5c4)n3)cc2)cc1 ; c1ccc(-c2ccc3oc4c(ccc5cccc(-c6nc(-c7ccccc7)nc(-c7ccc8c(c7)C(c7ccccc7)(c7ccccc7)c7ccccc7-8)n6)c54)c3c2)cc1 ; c1ccc(-c2cccc(-c3cccc4oc5c6cccc(-c7nc(-c8ccccc8)nc(-c8ccc9ccccc9c8)n7)c6ccc5c34)c2)cc1]

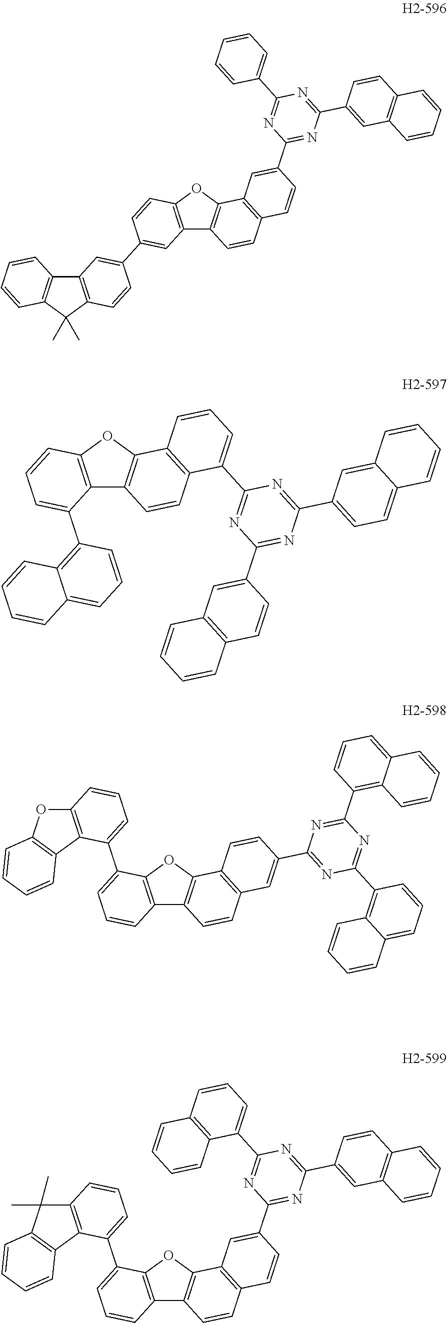 [SMILES: CC1(C)c2ccccc2-c2cc(-c3ccc4oc5c6cc(-c7nc(-c8ccccc8)nc(-c8ccc9ccccc9c8)n7)ccc6ccc5c4c3)ccc21 ; c1ccc2cc(-c3nc(-c4ccc5ccccc5c4)nc(-c4cccc5c4ccc4c5oc5cccc(-c6cccc7ccccc67)c54)n3)ccc2c1 ; c1ccc2c(-c3nc(-c4ccc5c(ccc6c7cccc(-c8cccc9oc%10ccccc%10c89)c7oc56)c4)nc(-c4cccc5ccccc45)n3)cccc2c1 ; CC1(C)c2ccccc2-c2c(-c3cccc4c3oc3c5cc(-c6nc(-c7ccc8ccccc8c7)nc(-c7cccc8ccccc78)n6)ccc5ccc43)cccc21]

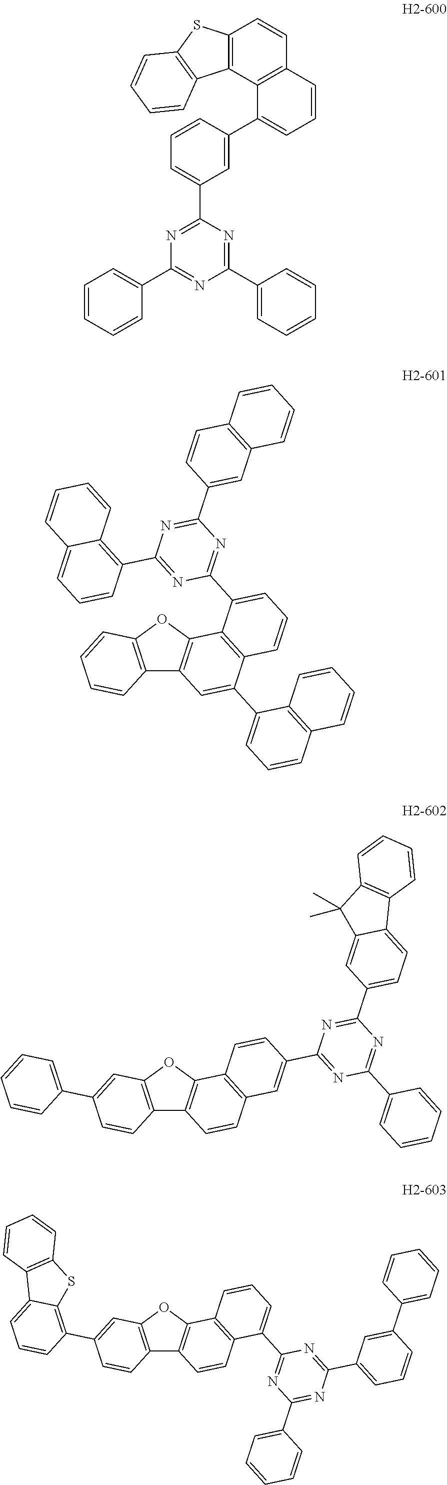 [SMILES: c1ccc(-c2nc(-c3ccccc3)nc(-c3cccc(-c4cccc5ccc6sc7ccccc7c6c45)c3)n2)cc1 ; c1ccc2cc(-c3nc(-c4cccc5ccccc45)nc(-c4cccc5c(-c6cccc7ccccc67)cc6c7ccccc7oc6c45)n3)ccc2c1 ; CC1(C)c2ccccc2-c2ccc(-c3nc(-c4ccccc4)nc(-c4ccc5c(ccc6c7ccc(-c8ccccc8)cc7oc56)c4)n3)cc21 ; c1ccc(-c2cccc(-c3nc(-c4ccccc4)nc(-c4cccc5c4ccc4c6ccc(-c7cccc8c7sc7ccccc78)cc6oc54)n3)c2)cc1]

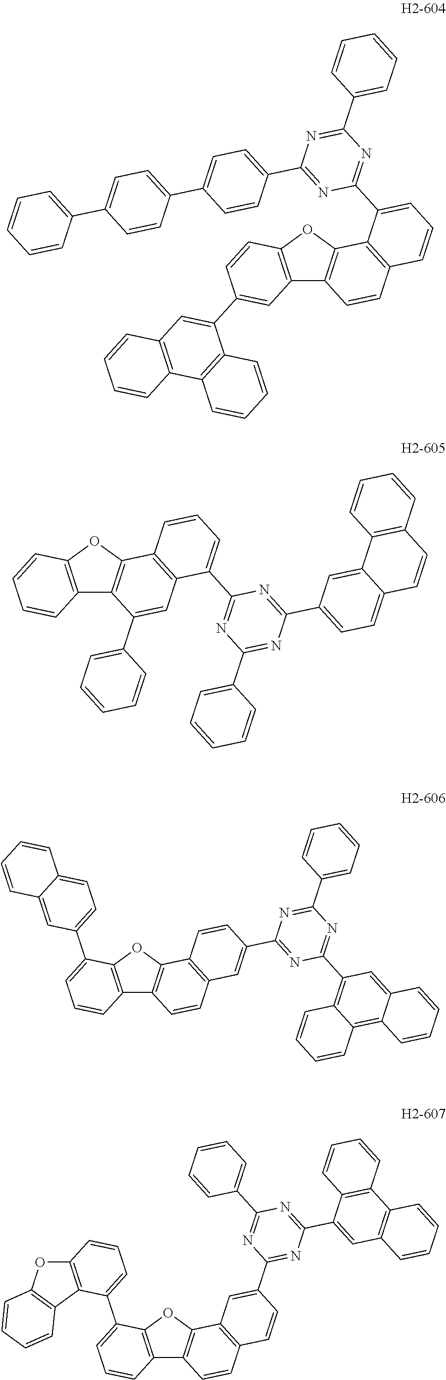 [SMILES: c1ccc(-c2ccc(-c3ccc(-c4nc(-c5ccccc5)nc(-c5cccc6ccc7c8cc(-c9cc%10ccccc%10c%10ccccc9%10)ccc8oc7c56)n4)cc3)cc2)cc1 ; c1ccc(-c2nc(-c3ccc4ccc5ccccc5c4c3)nc(-c3cccc4c3cc(-c3ccccc3)c3c5ccccc5oc43)n2)cc1 ; c1ccc(-c2nc(-c3ccc4c(ccc5c6cccc(-c7ccc8ccccc8c7)c6oc45)c3)nc(-c3cc4ccccc4c4ccccc34)n2)cc1 ; c1ccc(-c2nc(-c3ccc4ccc5c6cccc(-c7cccc8oc9ccccc9c78)c6oc5c4c3)nc(-c3cc4ccccc4c4ccccc34)n2)cc1]

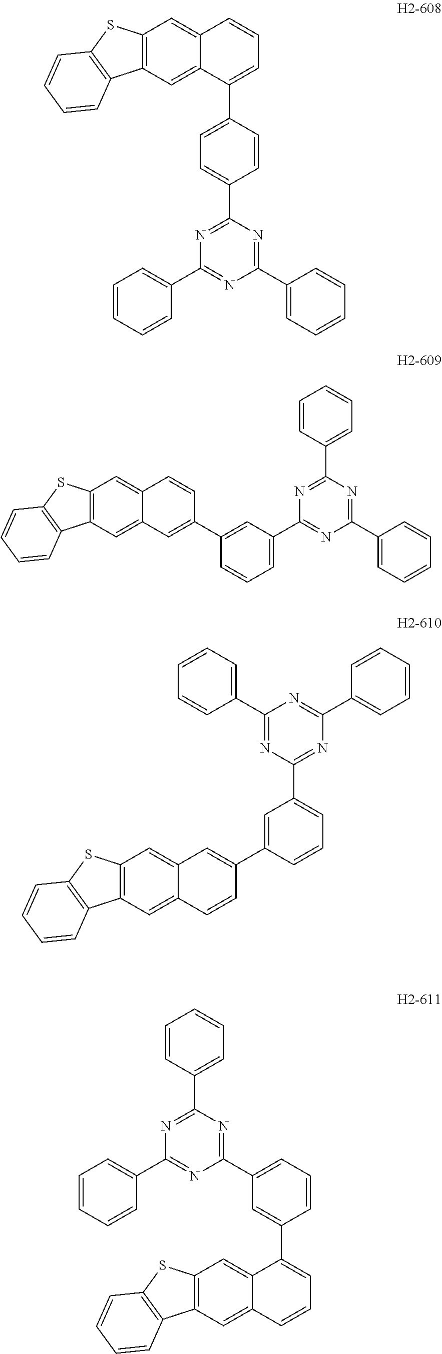 [SMILES: c1ccc(-c2nc(-c3ccccc3)nc(-c3ccc(-c4cccc5cc6sc7ccccc7c6cc45)cc3)n2)cc1 ; c1ccc(-c2nc(-c3ccccc3)nc(-c3cccc(-c4ccc5cc6sc7ccccc7c6cc5c4)c3)n2)cc1 ; c1ccc(-c2nc(-c3ccccc3)nc(-c3cccc(-c4ccc5cc6c(cc5c4)sc4ccccc46)c3)n2)cc1 ; c1ccc(-c2nc(-c3ccccc3)nc(-c3cccc(-c4cccc5cc6c(cc45)sc4ccccc46)c3)n2)cc1]

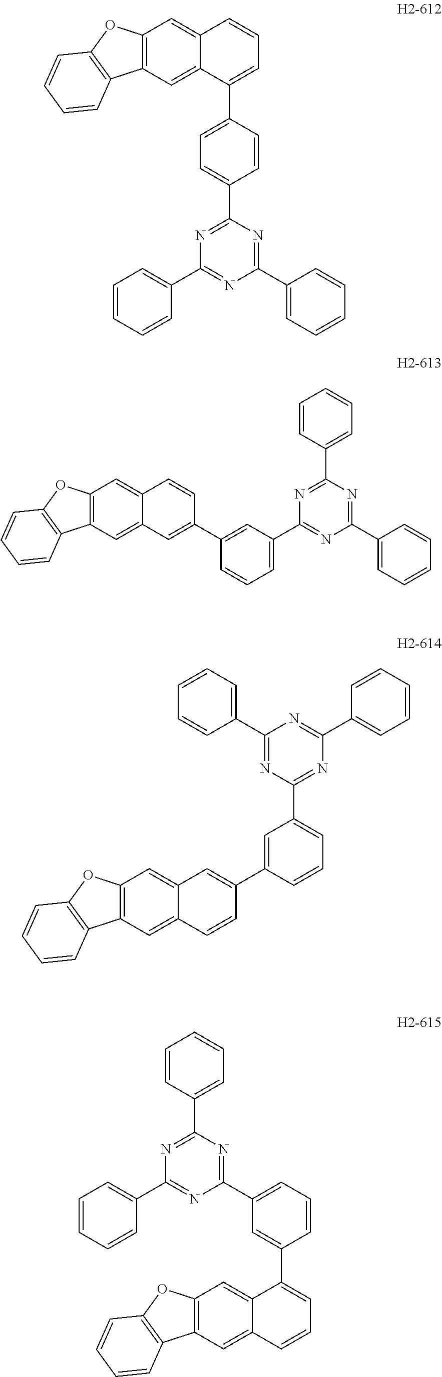 [SMILES: c1ccc(-c2nc(-c3ccccc3)nc(-c3ccc(-c4cccc5cc6oc7ccccc7c6cc45)cc3)n2)cc1 ; c1ccc(-c2nc(-c3ccccc3)nc(-c3cccc(-c4ccc5cc6oc7ccccc7c6cc5c4)c3)n2)cc1 ; c1ccc(-c2nc(-c3ccccc3)nc(-c3cccc(-c4ccc5cc6c(cc5c4)oc4ccccc46)c3)n2)cc1 ; c1ccc(-c2nc(-c3ccccc3)nc(-c3cccc(-c4cccc5cc6c(cc45)oc4ccccc46)c3)n2)cc1]

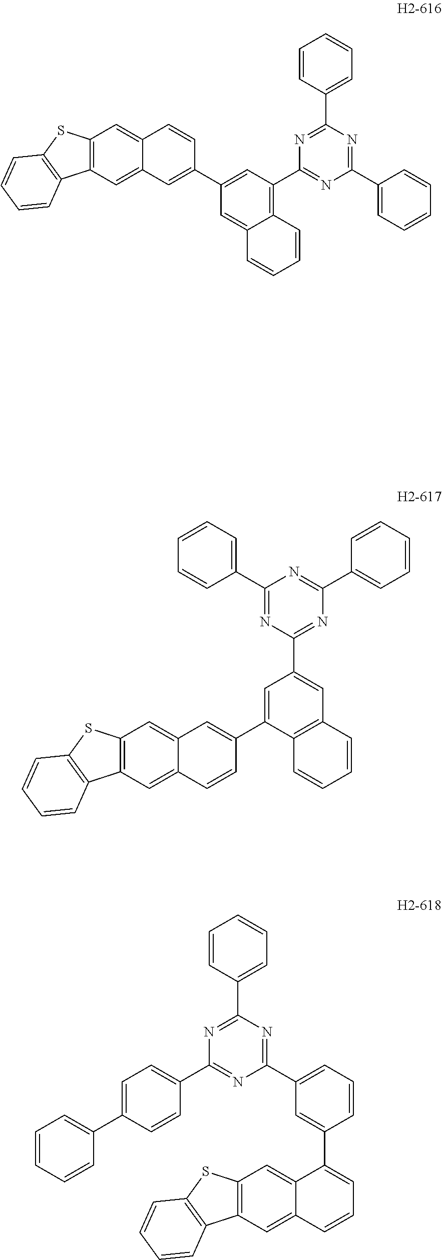 [SMILES: c1ccc(-c2nc(-c3ccccc3)nc(-c3cc(-c4ccc5cc6sc7ccccc7c6cc5c4)cc4ccccc34)n2)cc1 ; c1ccc(-c2nc(-c3ccccc3)nc(-c3cc(-c4ccc5cc6c(cc5c4)sc4ccccc46)c4ccccc4c3)n2)cc1 ; c1ccc(-c2ccc(-c3nc(-c4ccccc4)nc(-c4cccc(-c5cccc6cc7c(cc56)sc5ccccc57)c4)n3)cc2)cc1]

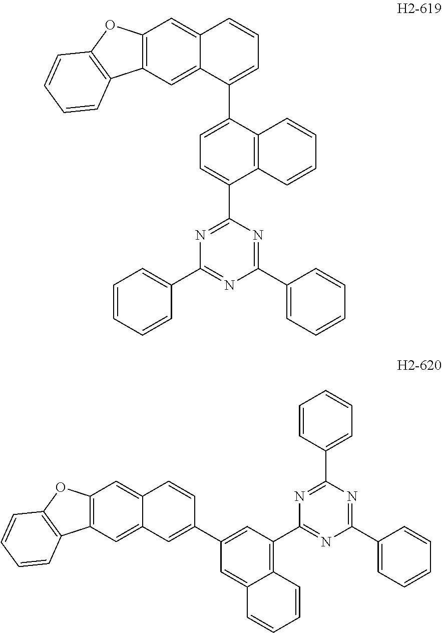 [SMILES: c1ccc(-c2nc(-c3ccccc3)nc(-c3ccc(-c4cccc5cc6oc7ccccc7c6cc45)c4ccccc34)n2)cc1 ; c1ccc(-c2nc(-c3ccccc3)nc(-c3cc(-c4ccc5cc6oc7ccccc7c6cc5c4)cc4ccccc34)n2)cc1]

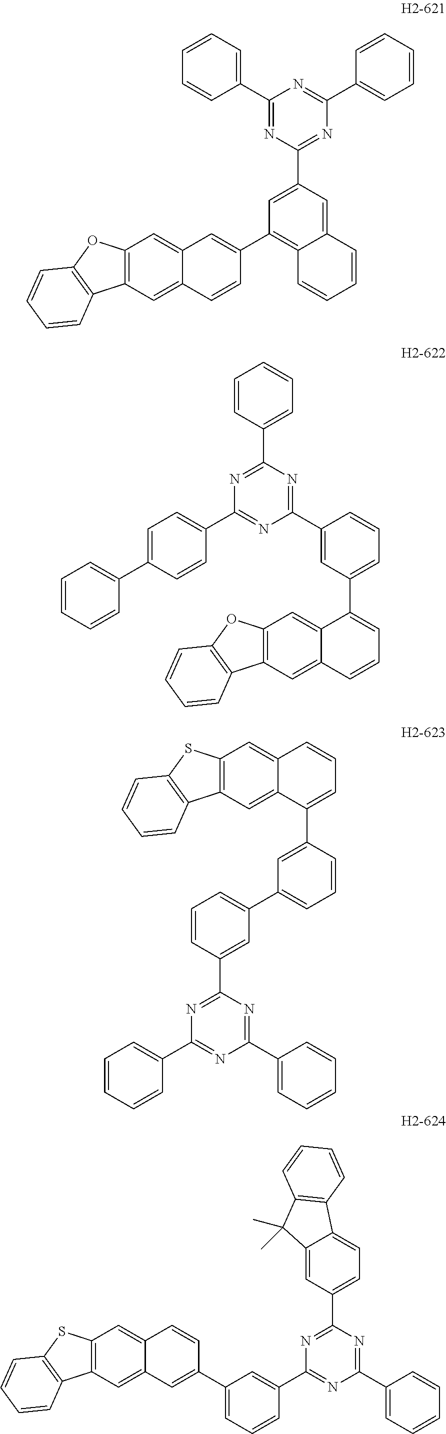

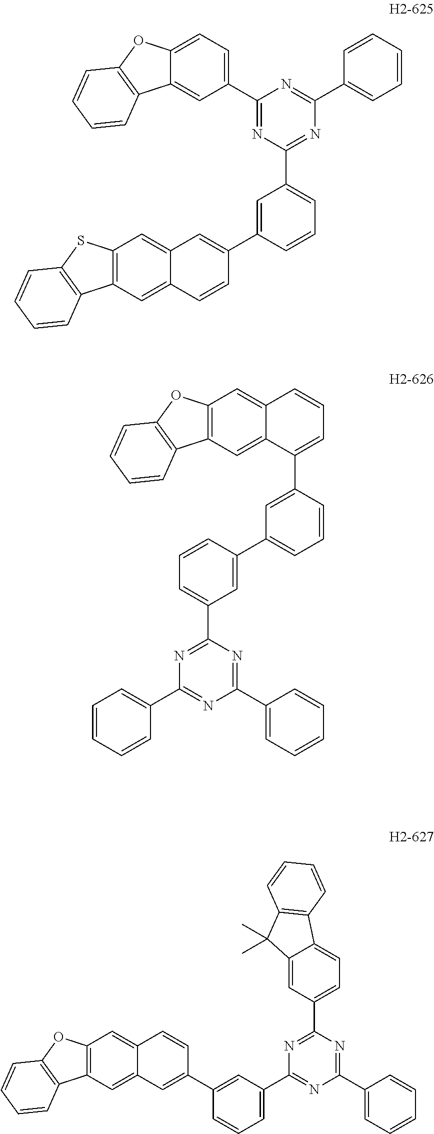 [SMILES: c1ccc(-c2nc(-c3cccc(-c4ccc5cc6c(cc5c4)sc4ccccc46)c3)nc(-c3ccc4oc5ccccc5c4c3)n2)cc1 ; c1ccc(-c2nc(-c3ccccc3)nc(-c3cccc(-c4cccc(-c5cccc6cc7oc8ccccc8c7cc56)c4)c3)n2)cc1 ; CC1(C)c2ccccc2-c2ccc(-c3nc(-c4ccccc4)nc(-c4cccc(-c5ccc6cc7oc8ccccc8c7cc6c5)c4)n3)cc21]

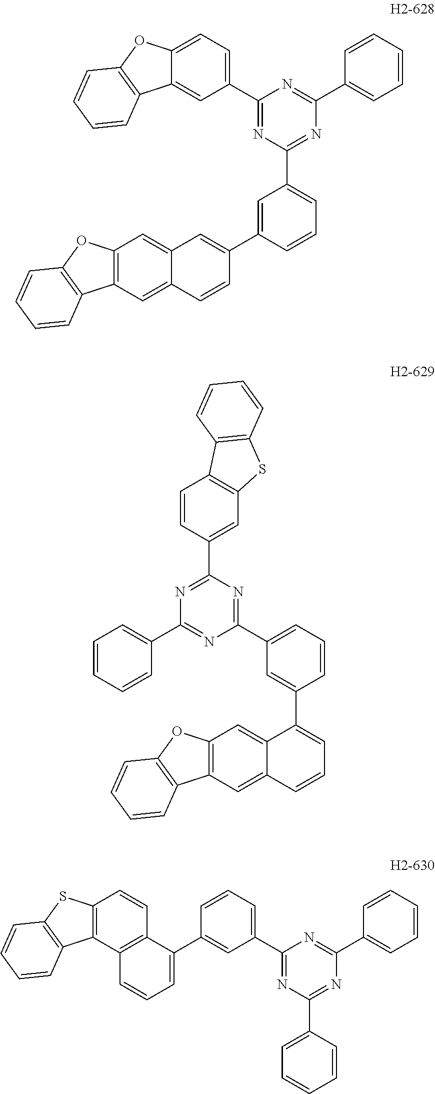 [SMILES: c1ccc(-c2nc(-c3cccc(-c4ccc5cc6c(cc5c4)oc4ccccc46)c3)nc(-c3ccc4oc5ccccc5c4c3)n2)cc1 ; c1ccc(-c2nc(-c3cccc(-c4cccc5cc6c(cc45)oc4ccccc46)c3)nc(-c3ccc4c(c3)sc3ccccc34)n2)cc1 ; c1ccc(-c2nc(-c3ccccc3)nc(-c3cccc(-c4cccc5c4ccc4sc6ccccc6c45)c3)n2)cc1]

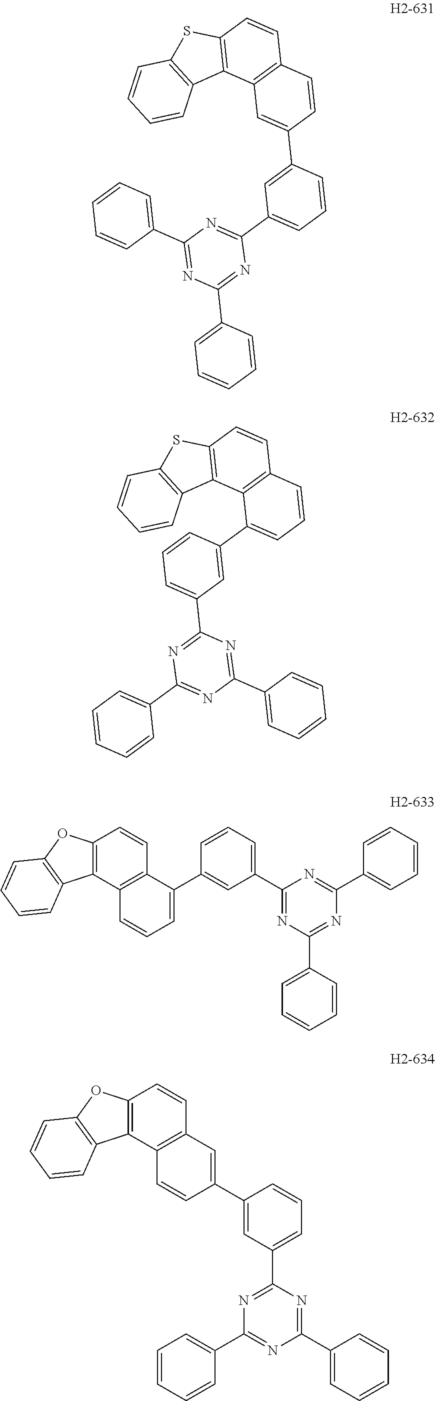

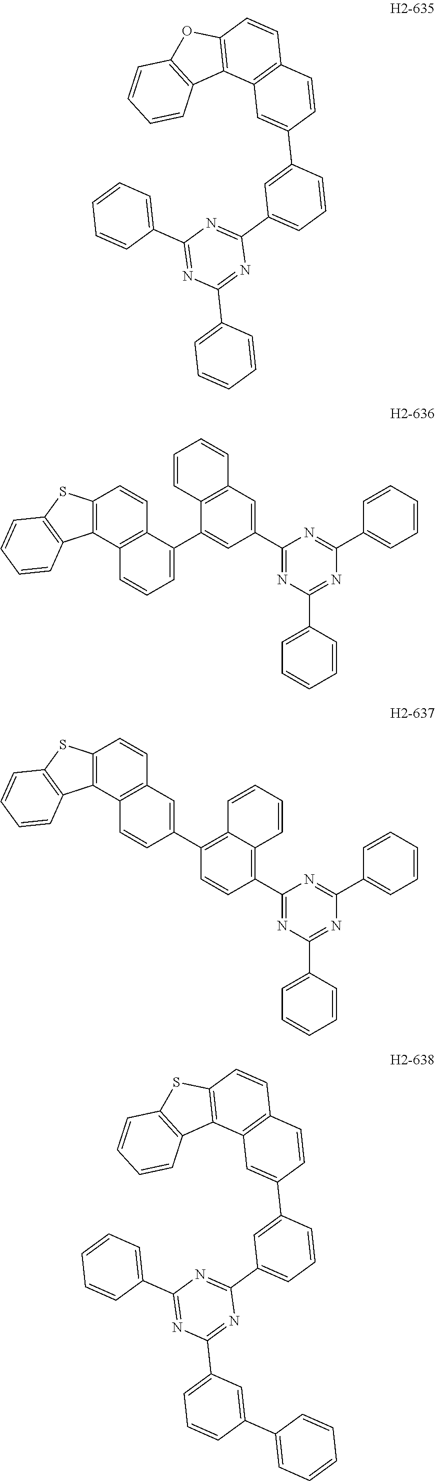 [SMILES: c1ccc(-c2nc(-c3ccccc3)nc(-c3cccc(-c4ccc5ccc6oc7ccccc7c6c5c4)c3)n2)cc1 ; c1ccc(-c2nc(-c3ccccc3)nc(-c3cc(-c4cccc5c4ccc4sc6ccccc6c45)c4ccccc4c3)n2)cc1 ; c1ccc(-c2nc(-c3ccccc3)nc(-c3ccc(-c4ccc5c(ccc6sc7ccccc7c65)c4)c4ccccc34)n2)cc1 ; c1ccc(-c2cccc(-c3nc(-c4ccccc4)nc(-c4cccc(-c5ccc6ccc7sc8ccccc8c7c6c5)c4)n3)c2)cc1]

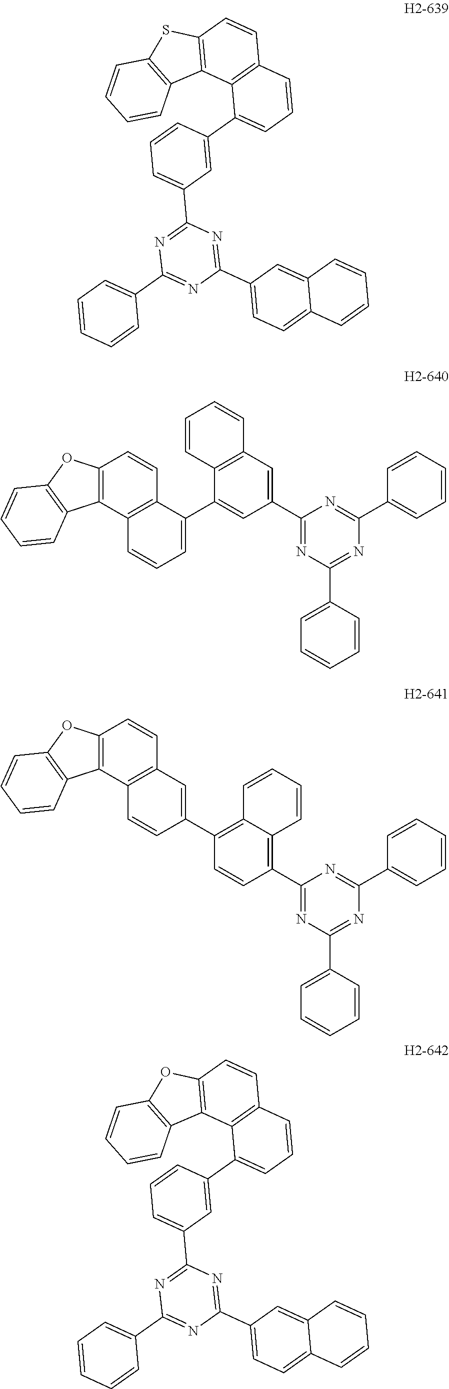 [SMILES: c1ccc(-c2nc(-c3cccc(-c4cccc5ccc6sc7ccccc7c6c45)c3)nc(-c3ccc4ccccc4c3)n2)cc1 ; c1ccc(-c2nc(-c3ccccc3)nc(-c3cc(-c4cccc5c4ccc4oc6ccccc6c45)c4ccccc4c3)n2)cc1 ; c1ccc(-c2nc(-c3ccccc3)nc(-c3ccc(-c4ccc5c(ccc6oc7ccccc7c65)c4)c4ccccc34)n2)cc1 ; c1ccc(-c2nc(-c3cccc(-c4cccc5ccc6oc7ccccc7c6c45)c3)nc(-c3ccc4ccccc4c3)n2)cc1]

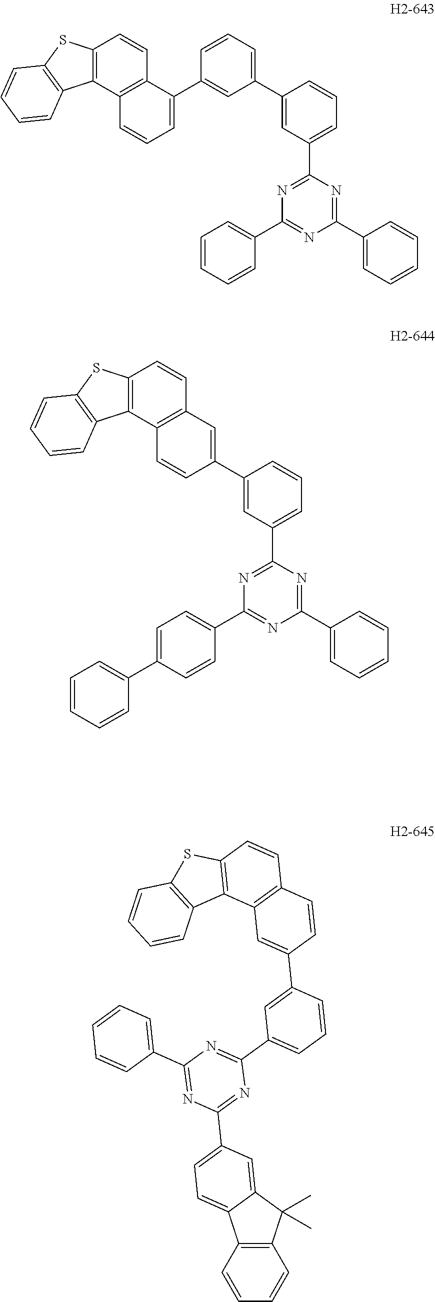 [SMILES: c1ccc(-c2nc(-c3ccccc3)nc(-c3cccc(-c4cccc(-c5cccc6c5ccc5sc7ccccc7c56)c4)c3)n2)cc1 ; c1ccc(-c2ccc(-c3nc(-c4ccccc4)nc(-c4cccc(-c5ccc6c(ccc7sc8ccccc8c76)c5)c4)n3)cc2)cc1 ; CC1(C)c2ccccc2-c2ccc(-c3nc(-c4ccccc4)nc(-c4cccc(-c5ccc6ccc7sc8ccccc8c7c6c5)c4)n3)cc21]

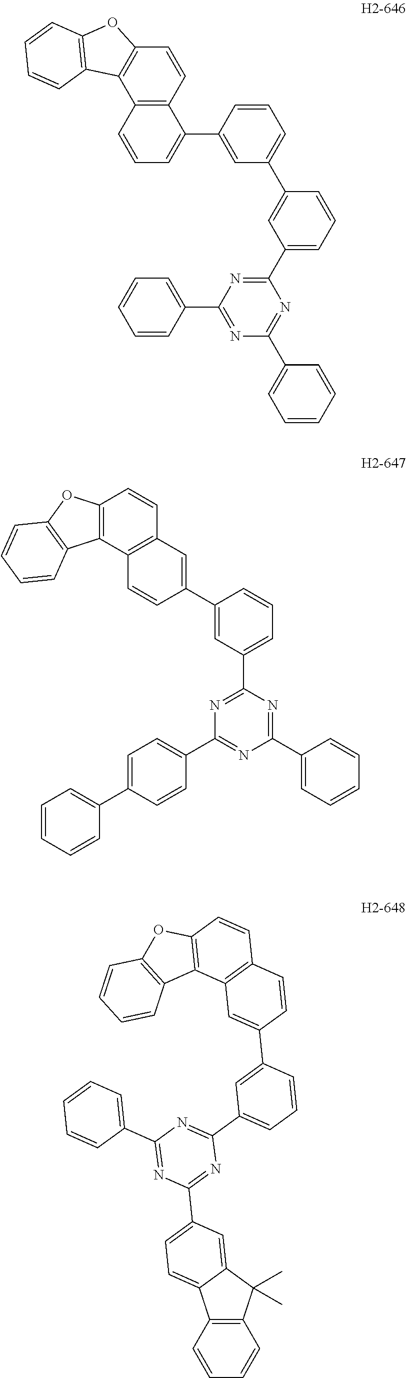 [SMILES: c1ccc(-c2nc(-c3ccccc3)nc(-c3cccc(-c4cccc(-c5cccc6c5ccc5oc7ccccc7c56)c4)c3)n2)cc1 ; c1ccc(-c2ccc(-c3nc(-c4ccccc4)nc(-c4cccc(-c5ccc6c(ccc7oc8ccccc8c76)c5)c4)n3)cc2)cc1 ; CC1(C)c2ccccc2-c2ccc(-c3nc(-c4ccccc4)nc(-c4cccc(-c5ccc6ccc7oc8ccccc8c7c6c5)c4)n3)cc21]

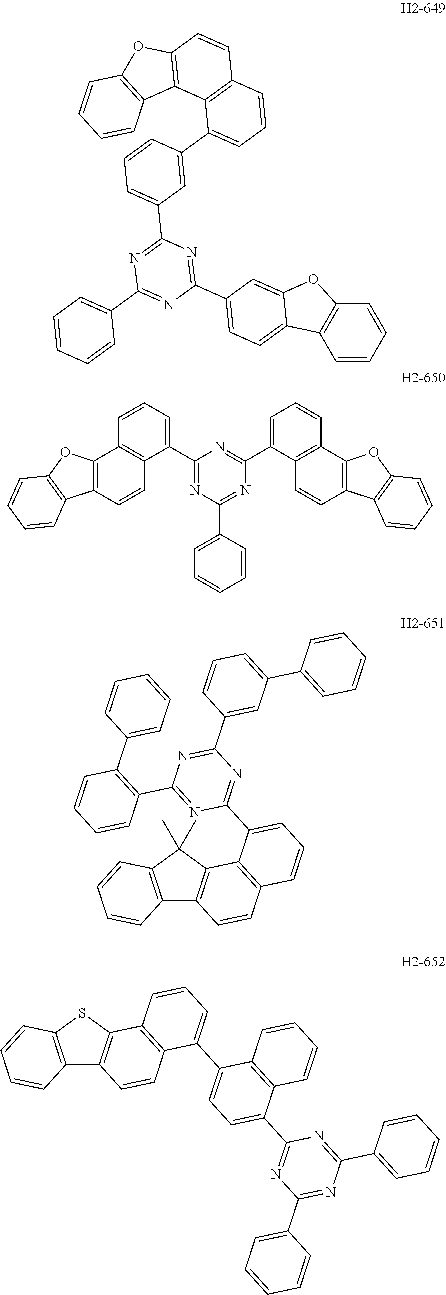 [SMILES: c1ccc(-c2nc(-c3cccc(-c4cccc5ccc6oc7ccccc7c6c45)c3)nc(-c3ccc4c(c3)oc3ccccc34)n2)cc1 ; c1ccc(-c2nc(-c3cccc4c3ccc3c5ccccc5oc43)nc(-c3cccc4c3ccc3c5ccccc5oc43)n2)cc1 ; CC1(C)c2ccccc2-c2ccc3cccc(-c4nc(-c5cccc(-c6ccccc6)c5)nc(-c5ccccc5-c5ccccc5)n4)c3c21 ; c1ccc(-c2nc(-c3ccccc3)nc(-c3ccc(-c4cccc5c4ccc4c6ccccc6sc54)c4ccccc34)n2)cc1]

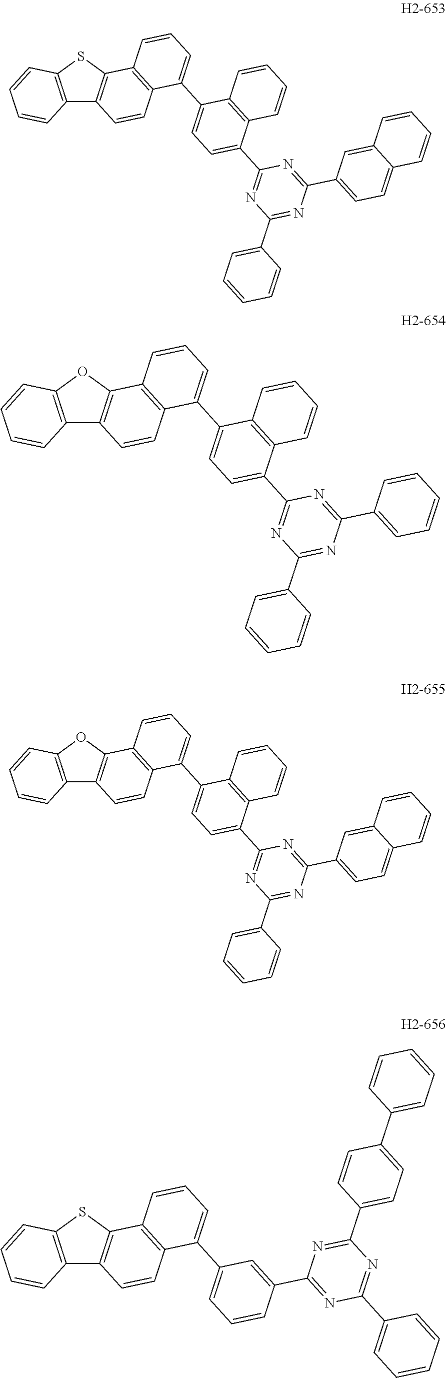 [SMILES: c1ccc(-c2nc(-c3ccc4ccccc4c3)nc(-c3ccc(-c4cccc5c4ccc4c6ccccc6sc54)c4ccccc34)n2)cc1 ; c1ccc(-c2nc(-c3ccccc3)nc(-c3ccc(-c4cccc5c4ccc4c6ccccc6oc54)c4ccccc34)n2)cc1 ; c1ccc(-c2nc(-c3ccc4ccccc4c3)nc(-c3ccc(-c4cccc5c4ccc4c6ccccc6oc54)c4ccccc34)n2)cc1 ; c1ccc(-c2ccc(-c3nc(-c4ccccc4)nc(-c4cccc(-c5cccc6c5ccc5c7ccccc7sc65)c4)n3)cc2)cc1]

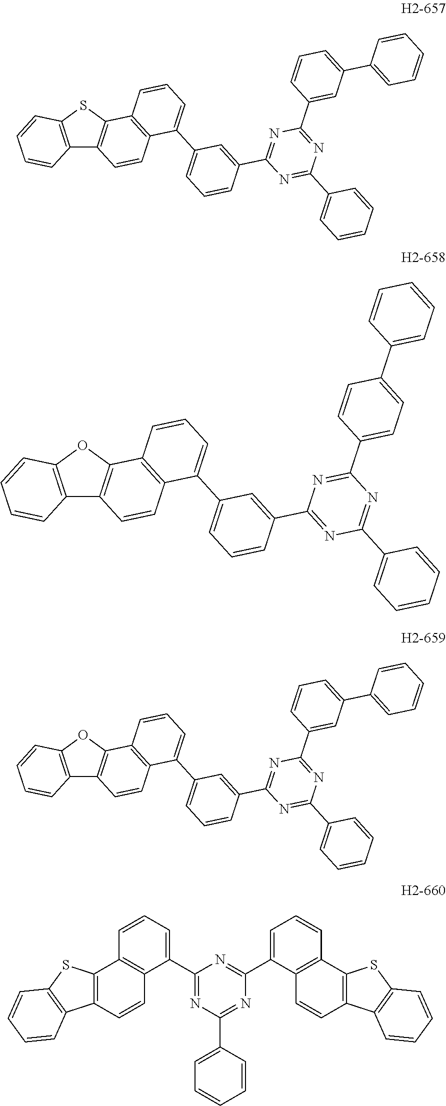 [SMILES: c1ccc(-c2cccc(-c3nc(-c4ccccc4)nc(-c4cccc(-c5cccc6c5ccc5c7ccccc7sc65)c4)n3)c2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2cccc(-c3nc(-c4ccccc4)nc(-c4cccc(-c5cccc6c5ccc5c7ccccc7oc65)c4)n3)c2)cc1 ; c1ccc(-c2nc(-c3cccc4c3ccc3c5ccccc5sc43)nc(-c3cccc4c3ccc3c5ccccc5sc43)n2)cc1]

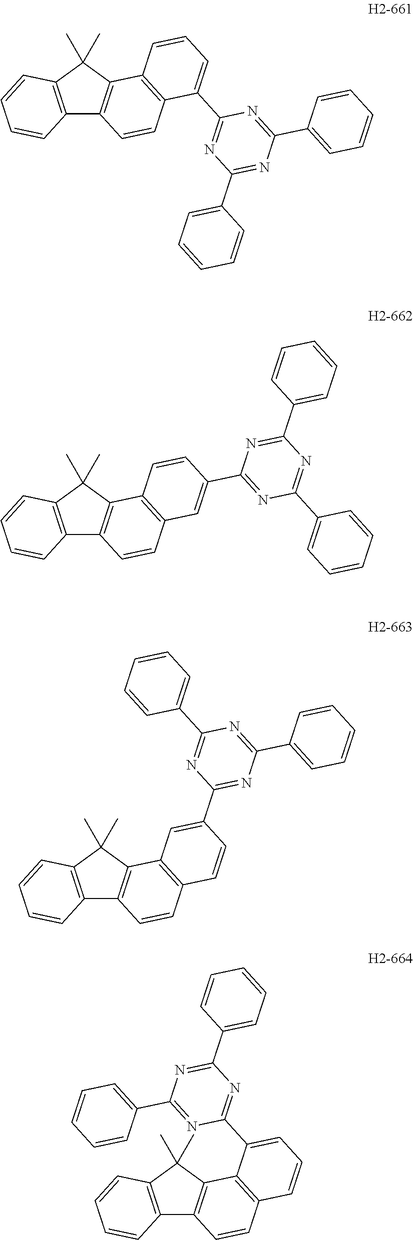 [SMILES: CC1(C)c2ccccc2-c2ccc3c(-c4nc(-c5ccccc5)nc(-c5ccccc5)n4)cccc3c21 ; CC1(C)c2ccccc2-c2ccc3cc(-c4nc(-c5ccccc5)nc(-c5ccccc5)n4)ccc3c21 ; CC1(C)c2ccccc2-c2ccc3ccc(-c4nc(-c5ccccc5)nc(-c5ccccc5)n4)cc3c21 ; CC1(C)c2ccccc2-c2ccc3cccc(-c4nc(-c5ccccc5)nc(-c5ccccc5)n4)c3c21]

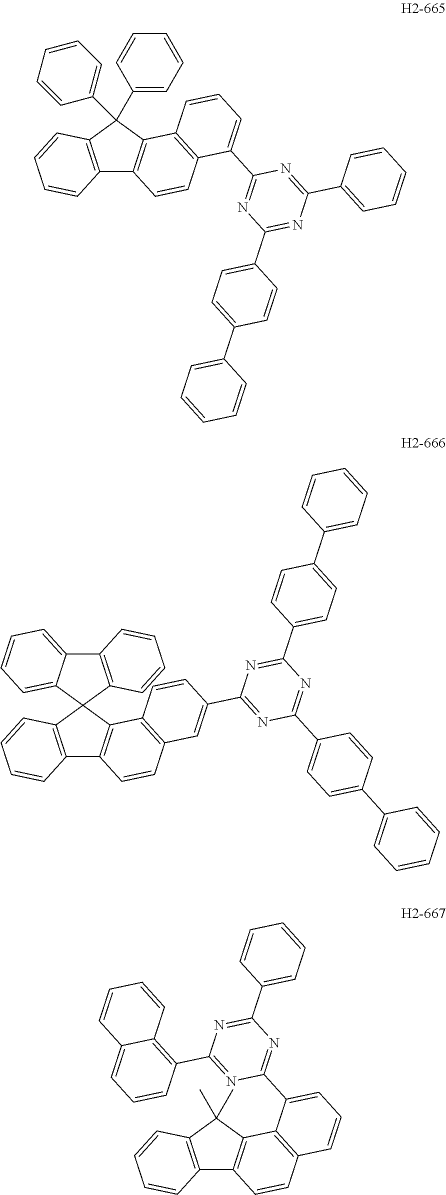 [SMILES: c1ccc(-c2ccc(-c3nc(-c4ccccc4)nc(-c4cccc5c6c(ccc45)-c4ccccc4C6(c4ccccc4)c4ccccc4)n3)cc2)cc1 ; c1ccc(-c2ccc(-c3nc(-c4ccc(-c5ccccc5)cc4)nc(-c4ccc5c6c(ccc5c4)-c4ccccc4C64c5ccccc5-c5ccccc54)n3)cc2)cc1 ; CC1(C)c2ccccc2-c2ccc3cccc(-c4nc(-c5ccccc5)nc(-c5cccc6ccccc56)n4)c3c21]

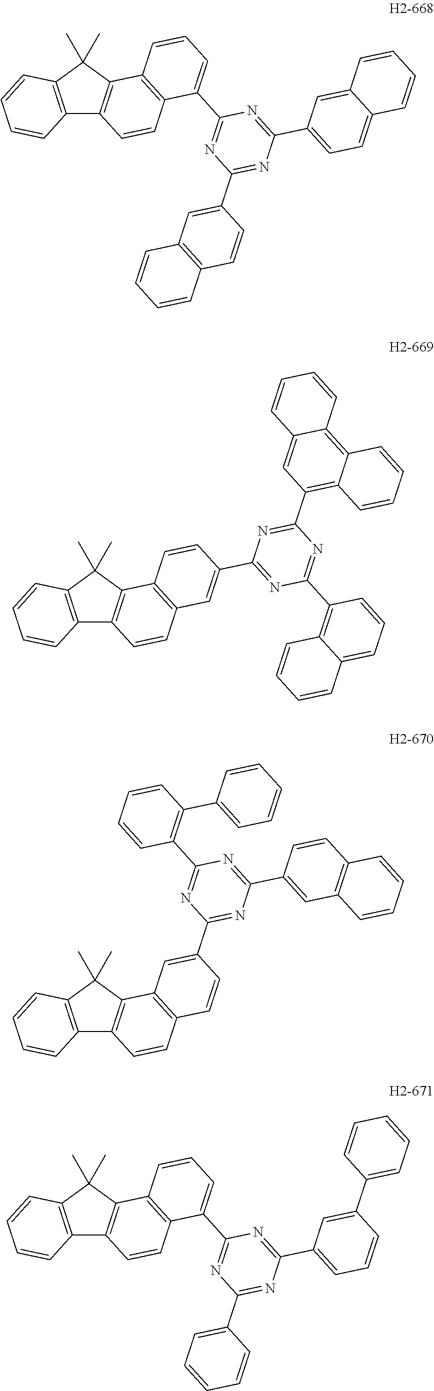 [SMILES: CC1(C)c2ccccc2-c2ccc3c(-c4nc(-c5ccc6ccccc6c5)nc(-c5ccc6ccccc6c5)n4)cccc3c21 ; CC1(C)c2ccccc2-c2ccc3cc(-c4nc(-c5cccc6ccccc56)nc(-c5cc6ccccc6c6ccccc56)n4)ccc3c21 ; CC1(C)c2ccccc2-c2ccc3ccc(-c4nc(-c5ccc6ccccc6c5)nc(-c5ccccc5-c5ccccc5)n4)cc3c21 ; CC1(C)c2ccccc2-c2ccc3c(-c4nc(-c5ccccc5)nc(-c5cccc(-c6ccccc6)c5)n4)cccc3c21]

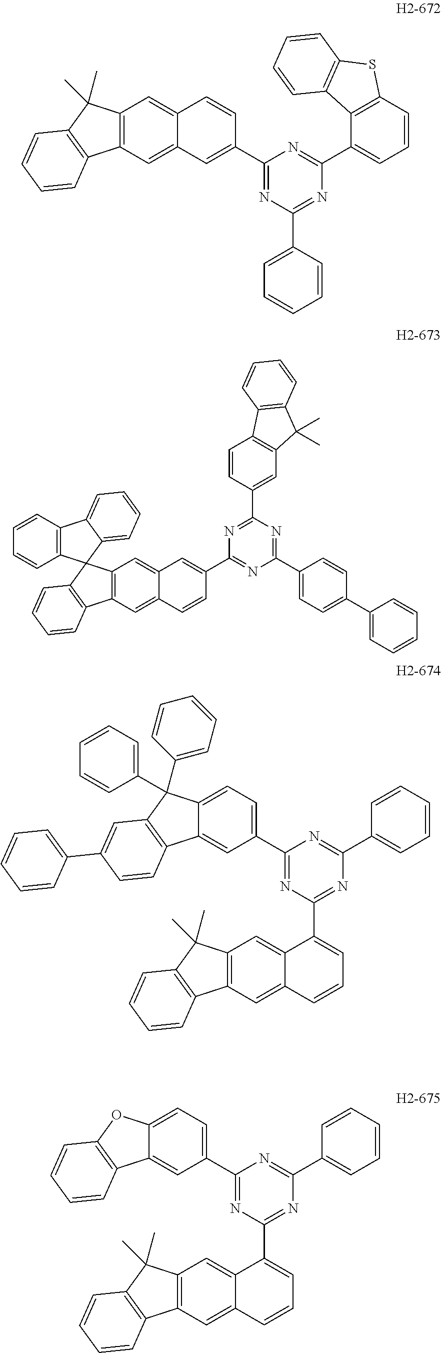 [SMILES: CC1(C)c2ccccc2-c2cc3cc(-c4nc(-c5ccccc5)nc(-c5cccc6sc7ccccc7c56)n4)ccc3cc21 ; CC1(C)c2ccccc2-c2ccc(-c3nc(-c4ccc(-c5ccccc5)cc4)nc(-c4ccc5cc6c(cc5c4)C4(c5ccccc5-c5ccccc54)c4ccccc4-6)n3)cc21 ; CC1(C)c2ccccc2-c2cc3cccc(-c4nc(-c5ccccc5)nc(-c5ccc6c(c5)-c5ccc(-c7ccccc7)cc5C6(c5ccccc5)c5ccccc5)n4)c3cc21 ; CC1(C)c2ccccc2-c2cc3cccc(-c4nc(-c5ccccc5)nc(-c5ccc6oc7ccccc7c6c5)n4)c3cc21]

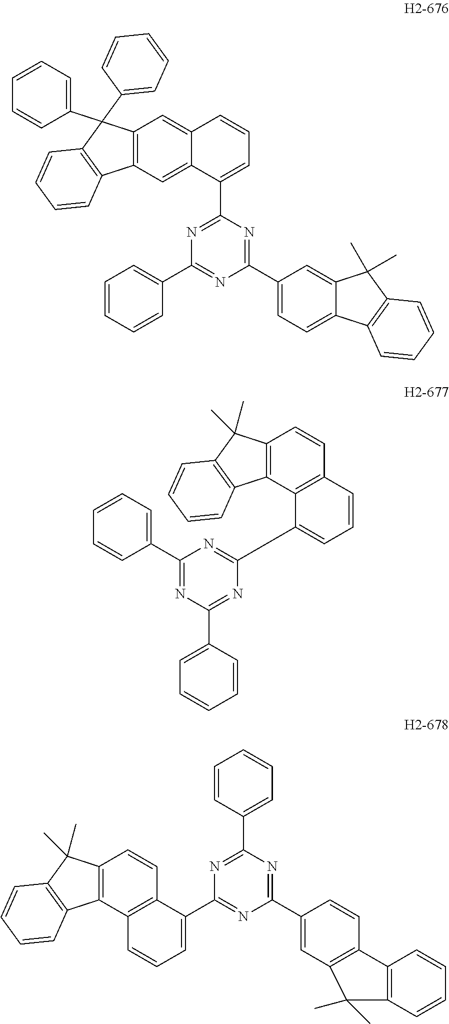 [SMILES: CC1(C)c2ccccc2-c2ccc(-c3nc(-c4ccccc4)nc(-c4cccc5cc6c(cc45)-c4ccccc4C6(c4ccccc4)c4ccccc4)n3)cc21 ; CC1(C)c2ccccc2-c2c1ccc1cccc(Cc3nc(-c4ccccc4)nc(-c4ccccc4)n3)c21 ; CC1(C)c2ccccc2-c2ccc(-c3nc(-c4ccccc4)nc(-c4cccc5c6c(ccc45)C(C)(C)c4ccccc4-6)n3)cc21]

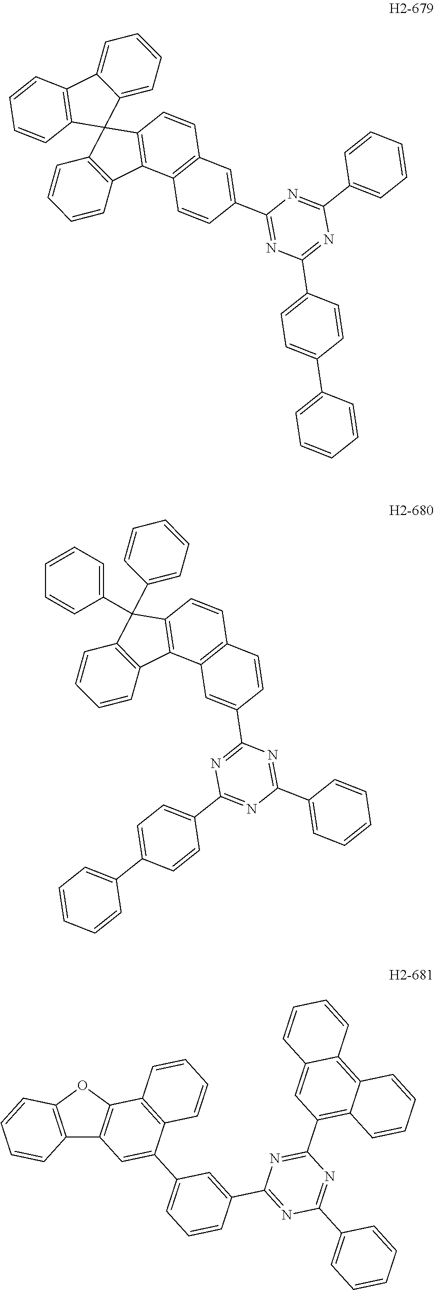 [SMILES: c1ccc(-c2ccc(-c3nc(-c4ccccc4)nc(-c4ccc5c6c(ccc5c4)C4(c5ccccc5-c5ccccc54)c4ccccc4-6)n3)cc2)cc1 ; c1ccc(-c2ccc(-c3nc(-c4ccccc4)nc(-c4ccc5ccc6c(c5c4)-c4ccccc4C6(c4ccccc4)c4ccccc4)n3)cc2)cc1 ; c1ccc(-c2nc(-c3cccc(-c4cc5c6ccccc6oc5c5ccccc45)c3)nc(-c3cc4ccccc4c4ccccc34)n2)cc1]

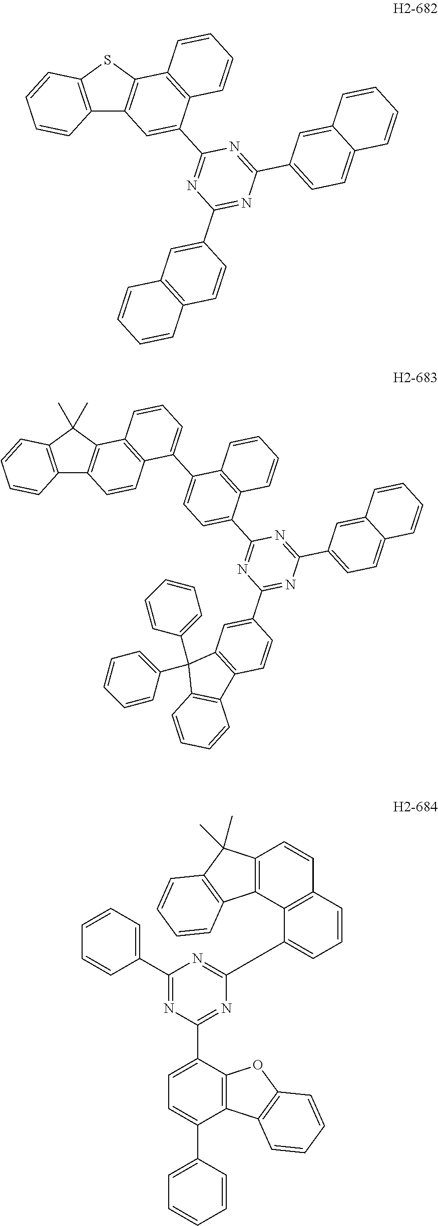 [SMILES: c1ccc2cc(-c3nc(-c4ccc5ccccc5c4)nc(-c4cc5c6ccccc6sc5c5ccccc45)n3)ccc2c1 ; CC1(C)c2ccccc2-c2ccc3c(-c4ccc(-c5nc(-c6ccc7c(c6)C(c6ccccc6)(c6ccccc6)c6ccccc6-7)nc(-c6ccc7ccccc7c6)n5)c5ccccc45)cccc3c21 ; CC1(C)c2ccccc2-c2c1ccc1cccc(Cc3nc(-c4ccccc4)nc(-c4ccc(-c5ccccc5)c5c4oc4ccccc45)n3)c21]

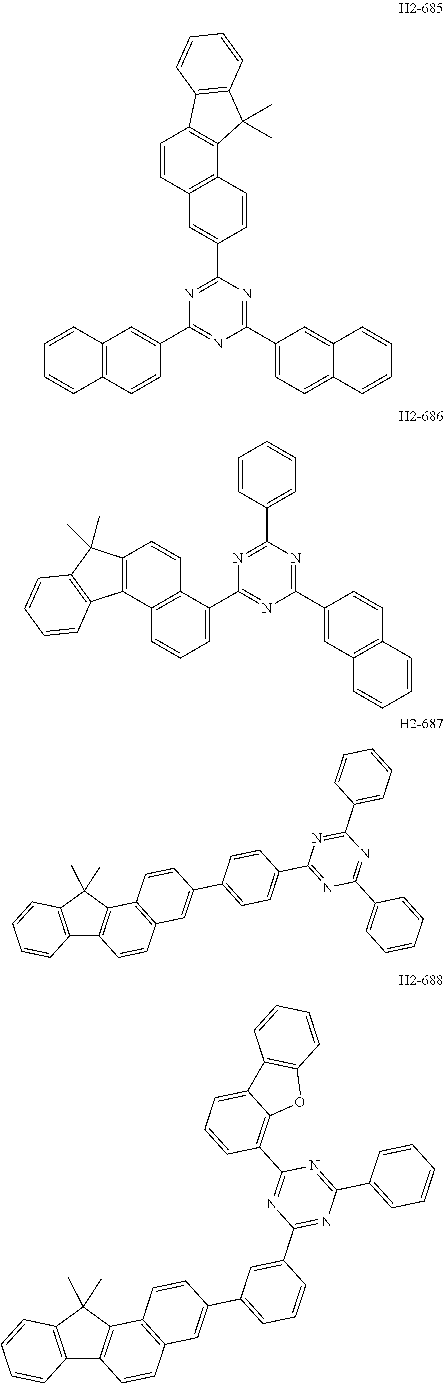

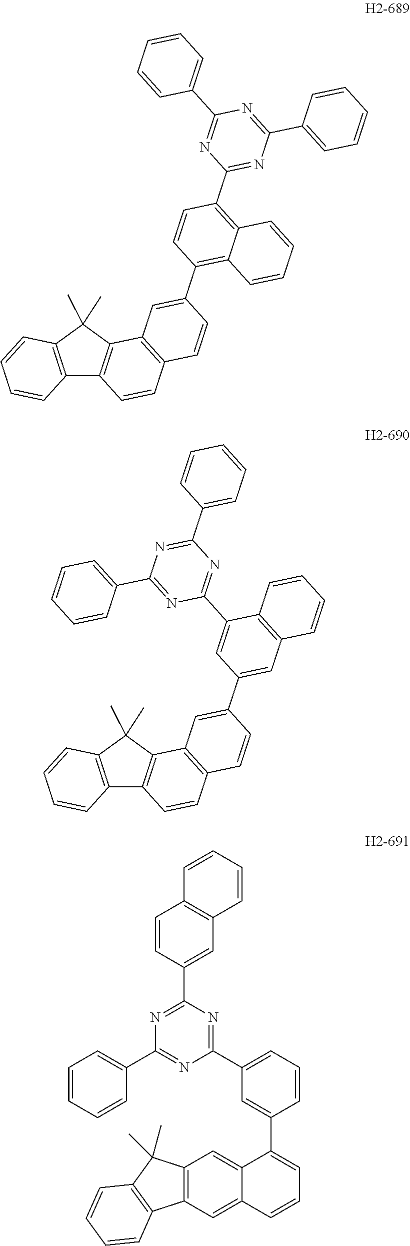 [SMILES: CC1(C)c2ccccc2-c2ccc3ccc(-c4ccc(-c5nc(-c6ccccc6)nc(-c6ccccc6)n5)c5ccccc45)cc3c21 ; CC1(C)c2ccccc2-c2ccc3ccc(-c4cc(-c5nc(-c6ccccc6)nc(-c6ccccc6)n5)c5ccccc5c4)cc3c21 ; CC1(C)c2ccccc2-c2cc3cccc(-c4cccc(-c5nc(-c6ccccc6)nc(-c6ccc7ccccc7c6)n5)c4)c3cc21]

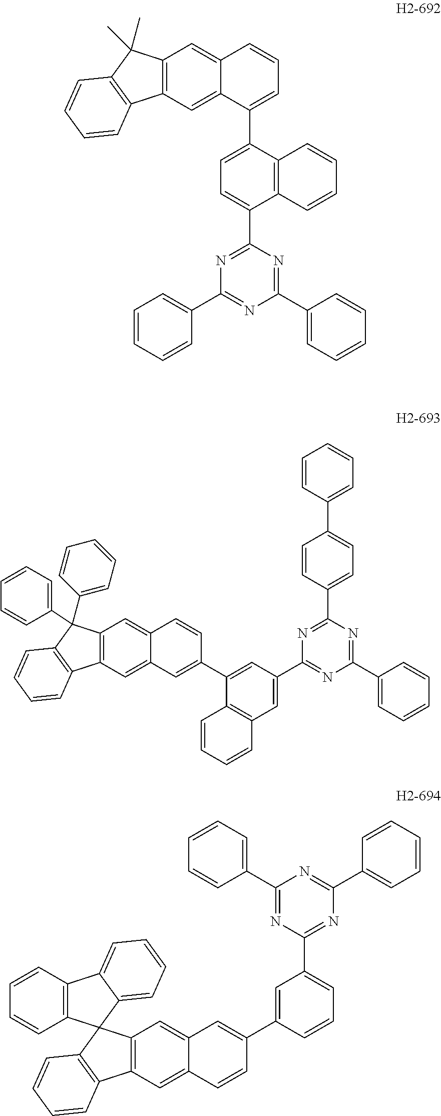 [SMILES: CC1(C)c2ccccc2-c2cc3c(-c4ccc(-c5nc(-c6ccccc6)nc(-c6ccccc6)n5)c5ccccc45)cccc3cc21 ; c1ccc(-c2ccc(-c3nc(-c4ccccc4)nc(-c4cc(-c5ccc6cc7c(cc6c5)-c5ccccc5C7(c5ccccc5)c5ccccc5)c5ccccc5c4)n3)cc2)cc1 ; c1ccc(-c2nc(-c3ccccc3)nc(-c3cccc(-c4ccc5cc6c(cc5c4)C4(c5ccccc5-c5ccccc54)c4ccccc4-6)c3)n2)cc1]

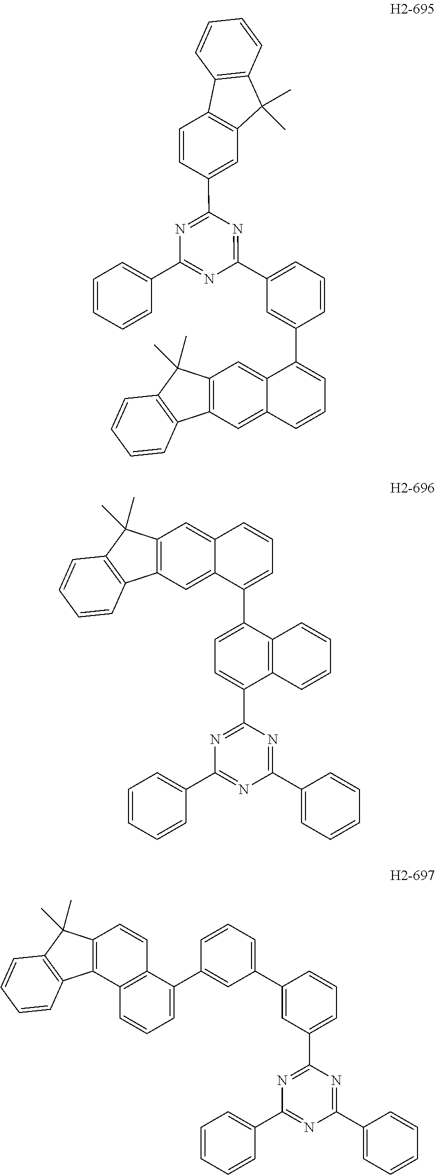

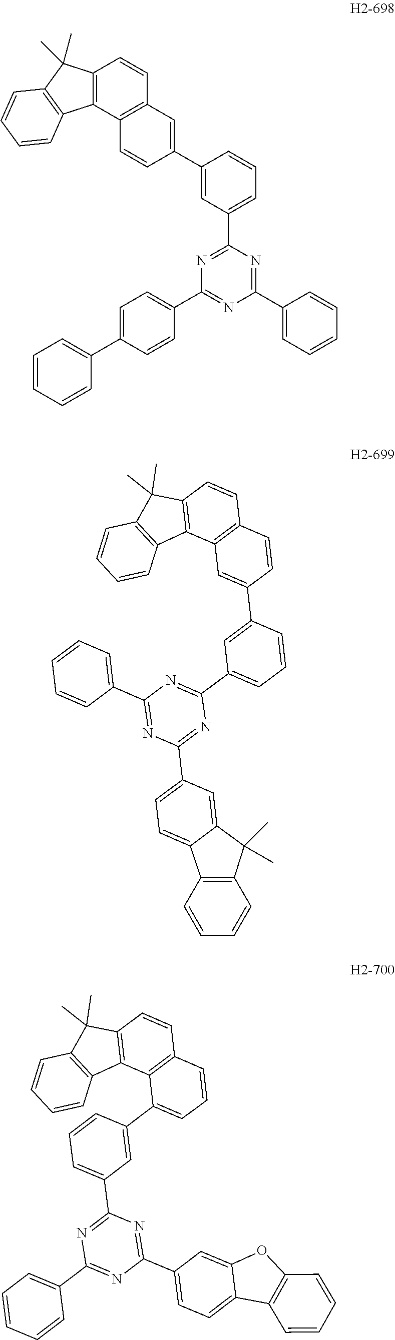 [SMILES: CC1(C)c2ccccc2-c2c1ccc1cc(-c3cccc(-c4nc(-c5ccccc5)nc(-c5ccc(-c6ccccc6)cc5)n4)c3)ccc21 ; CC1(C)c2ccccc2-c2ccc(-c3nc(-c4ccccc4)nc(-c4cccc(-c5ccc6ccc7c(c6c5)-c5ccccc5C7(C)C)c4)n3)cc21 ; CC1(C)c2ccccc2-c2c1ccc1cccc(-c3cccc(-c4nc(-c5ccccc5)nc(-c5ccc6c(c5)oc5ccccc56)n4)c3)c21]

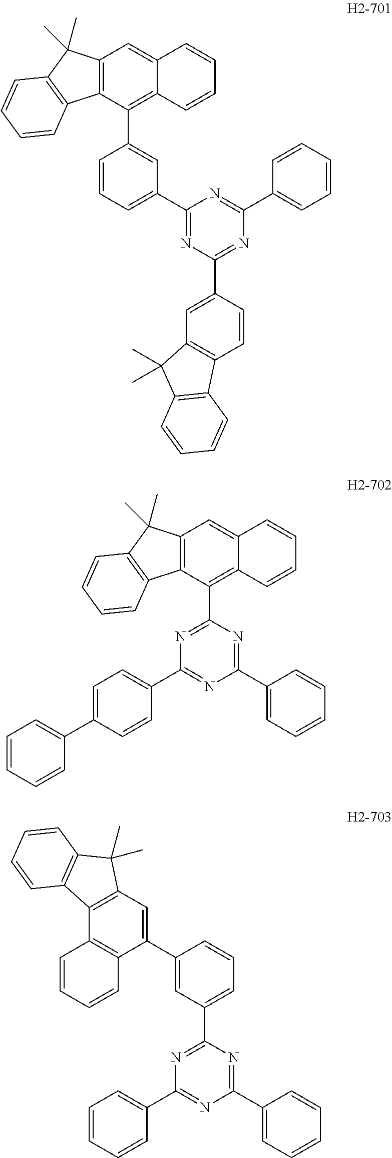

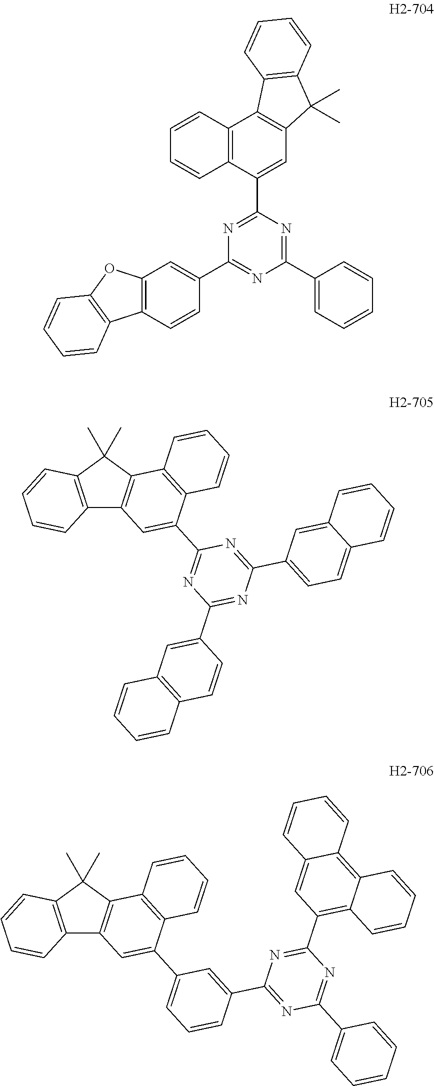 [SMILES: CC1(C)c2ccccc2-c2c1cc(-c1nc(-c3ccccc3)nc(-c3ccc4c(c3)oc3ccccc34)n1)c1ccccc21 ; CC1(C)c2ccccc2-c2cc(-c3nc(-c4ccc5ccccc5c4)nc(-c4ccc5ccccc5c4)n3)c3ccccc3c21 ; CC1(C)c2ccccc2-c2cc(-c3cccc(-c4nc(-c5ccccc5)nc(-c5cc6ccccc6c6ccccc56)n4)c3)c3ccccc3c21]

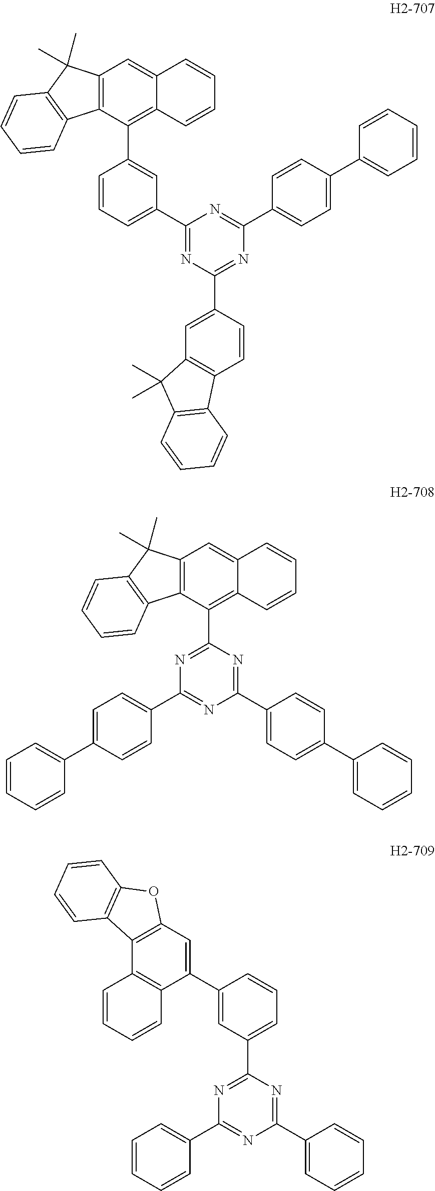 [SMILES: CC1(C)c2ccccc2-c2ccc(-c3nc(-c4ccc(-c5ccccc5)cc4)nc(-c4cccc(-c5c6c(cc7ccccc57)C(C)(C)c5ccccc5-6)c4)n3)cc21 ; CC1(C)c2ccccc2-c2c1cc1ccccc1c2-c1nc(-c2ccc(-c3ccccc3)cc2)nc(-c2ccc(-c3ccccc3)cc2)n1 ; c1ccc(-c2nc(-c3ccccc3)nc(-c3cccc(-c4cc5oc6ccccc6c5c5ccccc45)c3)n2)cc1]

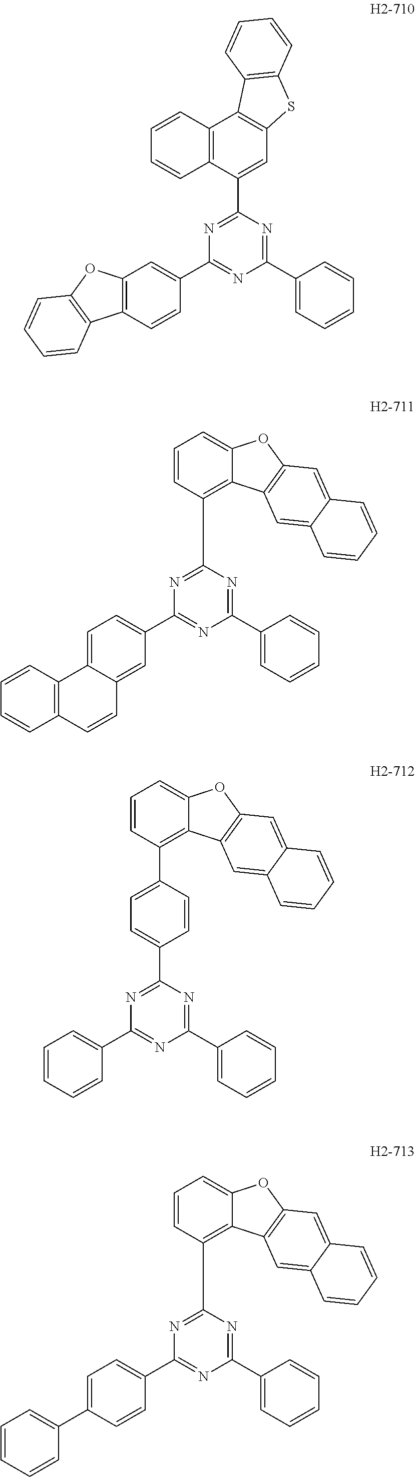 [SMILES: c1ccc(-c2nc(-c3ccc4c(c3)oc3ccccc34)nc(-c3cc4sc5ccccc5c4c4ccccc34)n2)cc1 ; c1ccc(-c2nc(Cc3cccc4oc5cc6ccccc6cc5c34)nc(-c3ccc4c(ccc5ccccc54)c3)n2)cc1 ; c1ccc(-c2nc(-c3ccccc3)nc(-c3ccc(-c4cccc5oc6cc7ccccc7cc6c45)cc3)n2)cc1 ; c1ccc(-c2ccc(-c3nc(Cc4cccc5oc6cc7ccccc7cc6c45)nc(-c4ccccc4)n3)cc2)cc1]

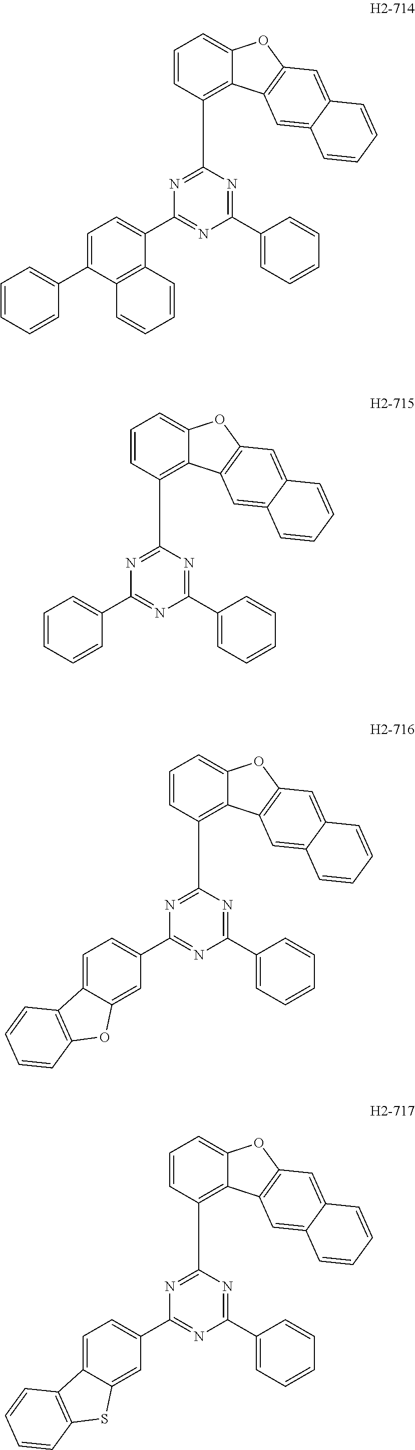 [SMILES: c1ccc(-c2nc(Cc3cccc4oc5cc6ccccc6cc5c34)nc(-c3ccc(-c4ccccc4)c4ccccc34)n2)cc1 ; c1ccc(-c2nc(Cc3cccc4oc5cc6ccccc6cc5c34)nc(-c3ccccc3)n2)cc1 ; c1ccc(-c2nc(Cc3cccc4oc5cc6ccccc6cc5c34)nc(-c3ccc4c(c3)oc3ccccc34)n2)cc1 ; c1ccc(-c2nc(Cc3cccc4oc5cc6ccccc6cc5c34)nc(-c3ccc4c(c3)sc3ccccc34)n2)cc1]

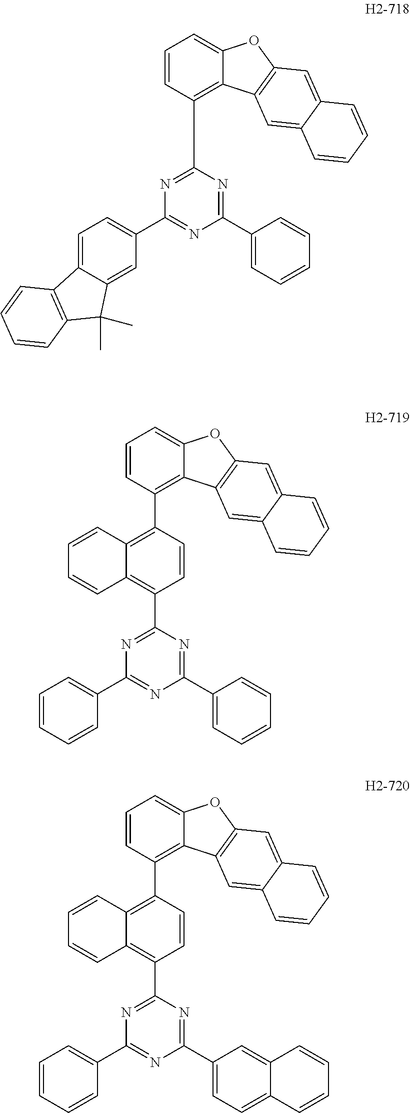 [SMILES: CC1(C)c2ccccc2-c2ccc(-c3nc(Cc4cccc5oc6cc7ccccc7cc6c45)nc(-c4ccccc4)n3)cc21 ; c1ccc(-c2nc(-c3ccccc3)nc(-c3ccc(-c4cccc5oc6cc7ccccc7cc6c45)c4ccccc34)n2)cc1 ; c1ccc(-c2nc(-c3ccc4ccccc4c3)nc(-c3ccc(-c4cccc5oc6cc7ccccc7cc6c45)c4ccccc34)n2)cc1]

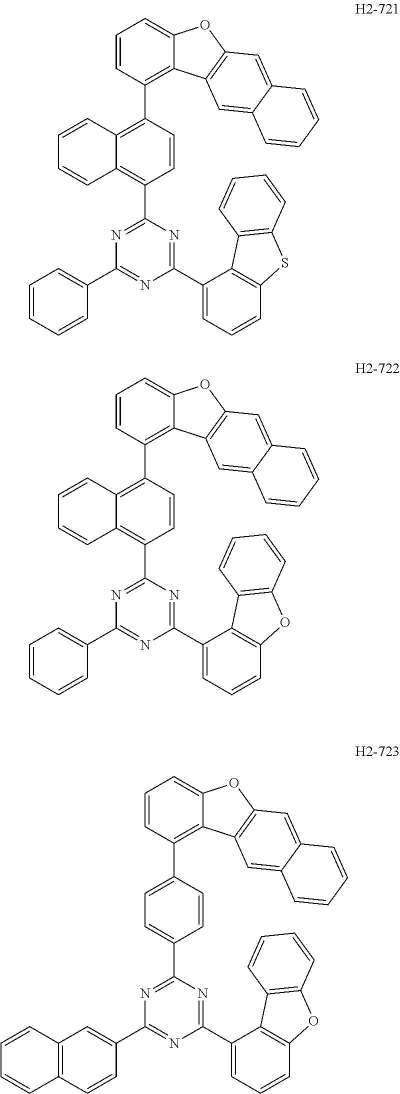 [SMILES: c1ccc(-c2nc(-c3ccc(-c4cccc5oc6cc7ccccc7cc6c45)c4ccccc34)nc(-c3cccc4sc5ccccc5c34)n2)cc1 ; c1ccc(-c2nc(-c3ccc(-c4cccc5oc6cc7ccccc7cc6c45)c4ccccc34)nc(-c3cccc4oc5ccccc5c34)n2)cc1 ; c1ccc2cc(-c3nc(-c4ccc(-c5cccc6oc7cc8ccccc8cc7c56)cc4)nc(-c4cccc5oc6ccccc6c45)n3)ccc2c1]

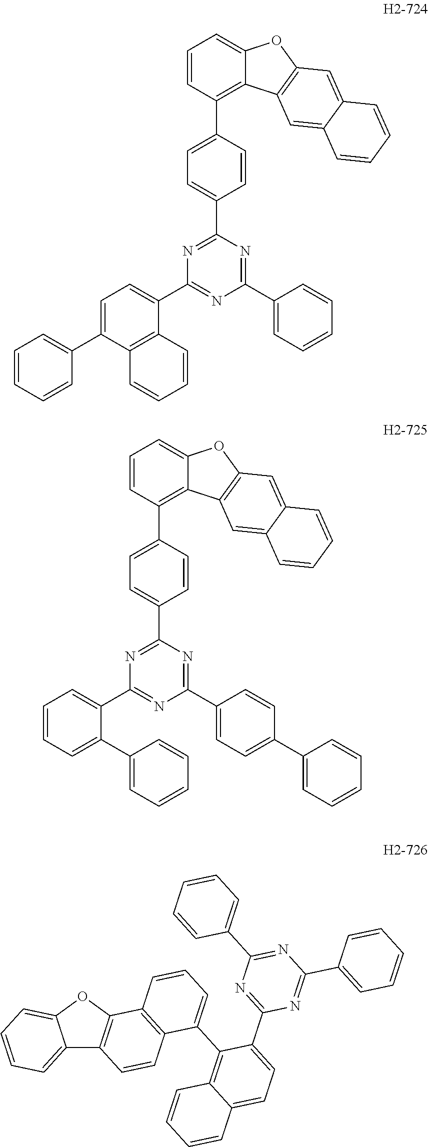

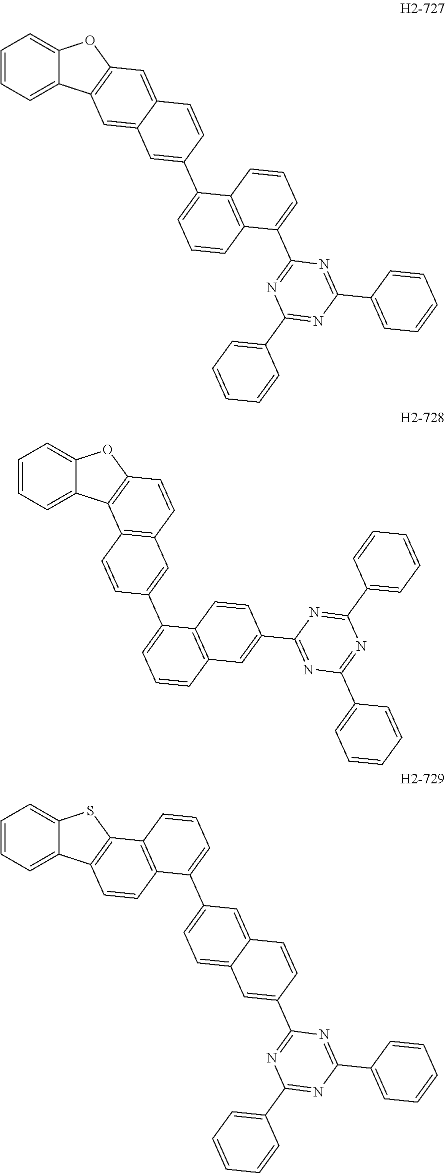 [SMILES: c1ccc(-c2nc(-c3ccccc3)nc(-c3cccc4c(-c5ccc6cc7oc8ccccc8c7cc6c5)cccc34)n2)cc1 ; c1ccc(-c2nc(-c3ccccc3)nc(-c3ccc4c(-c5ccc6c(ccc7oc8ccccc8c76)c5)cccc4c3)n2)cc1 ; c1ccc(-c2nc(-c3ccccc3)nc(-c3ccc4cc(-c5cccc6c5ccc5c7ccccc7sc65)ccc4c3)n2)cc1]

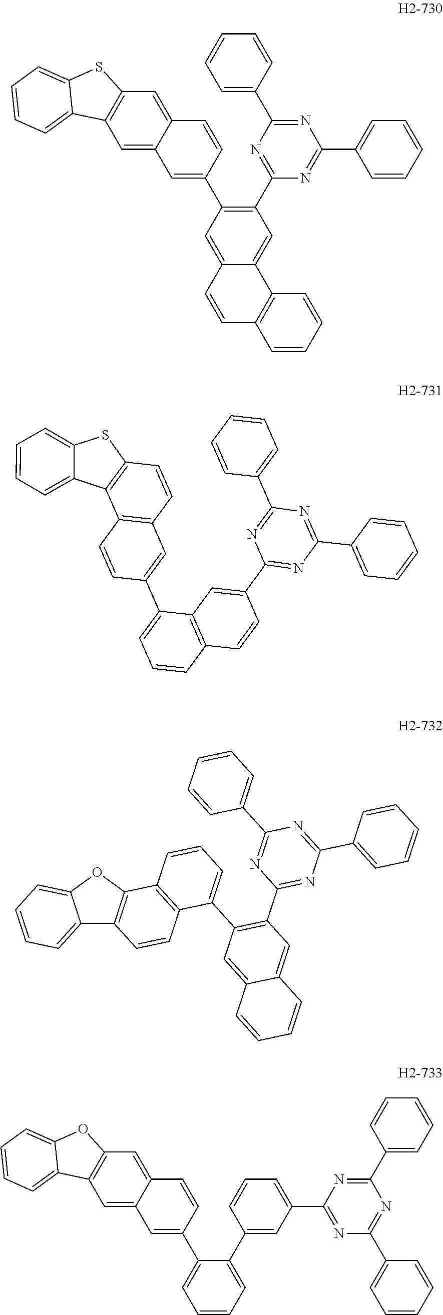 [SMILES: c1ccc(-c2nc(-c3ccccc3)nc(-c3cc4c(ccc5ccccc54)cc3-c3ccc4cc5sc6ccccc6c5cc4c3)n2)cc1 ; c1ccc(-c2nc(-c3ccccc3)nc(-c3ccc4cccc(-c5ccc6c(ccc7sc8ccccc8c76)c5)c4c3)n2)cc1 ; c1ccc(-c2nc(-c3ccccc3)nc(-c3cc4ccccc4cc3-c3cccc4c3ccc3c5ccccc5oc43)n2)cc1 ; c1ccc(-c2nc(-c3ccccc3)nc(-c3cccc(-c4ccccc4-c4ccc5cc6oc7ccccc7c6cc5c4)c3)n2)cc1]

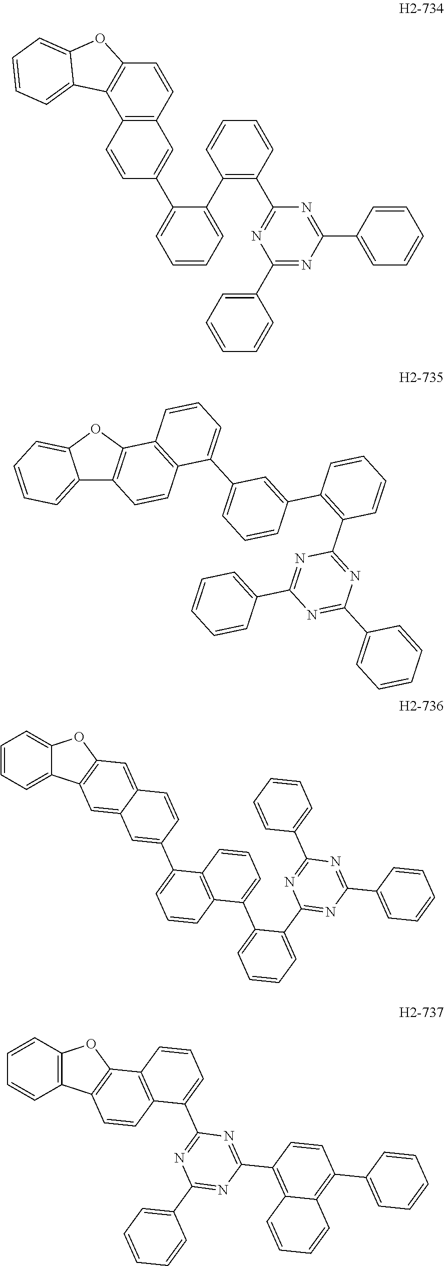 [SMILES: c1ccc(-c2nc(-c3ccccc3)nc(-c3ccccc3-c3ccccc3-c3ccc4c(ccc5oc6ccccc6c54)c3)n2)cc1 ; c1ccc(-c2nc(-c3ccccc3)nc(-c3ccccc3-c3cccc(-c4cccc5c4ccc4c6ccccc6oc54)c3)n2)cc1 ; c1ccc(-c2nc(-c3ccccc3)nc(-c3ccccc3-c3cccc4c(-c5ccc6cc7oc8ccccc8c7cc6c5)cccc34)n2)cc1 ; c1ccc(-c2nc(-c3ccc(-c4ccccc4)c4ccccc34)nc(-c3cccc4c3ccc3c5ccccc5oc43)n2)cc1]

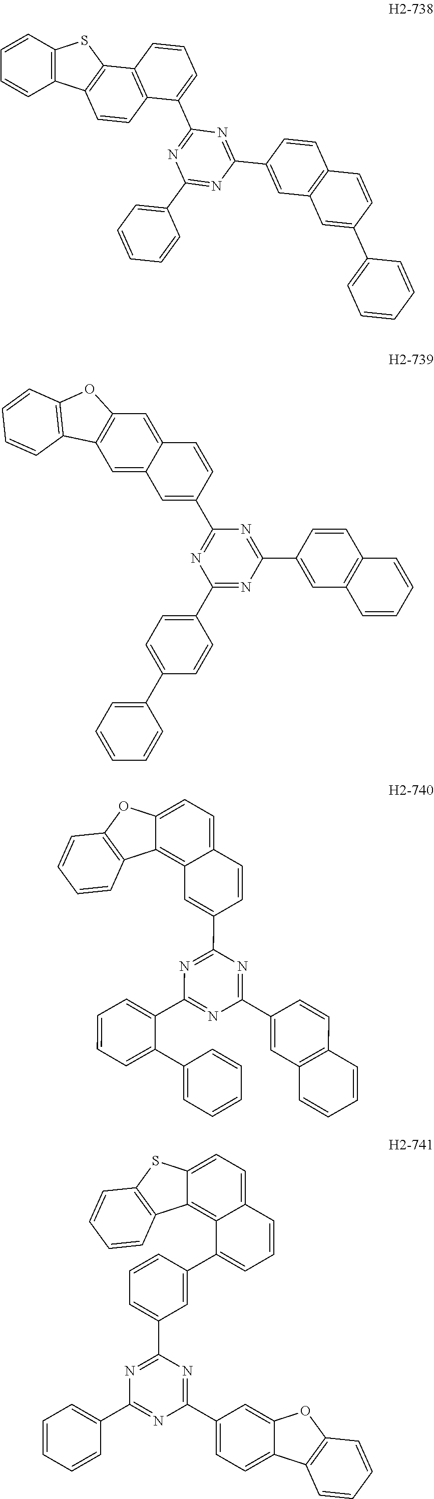

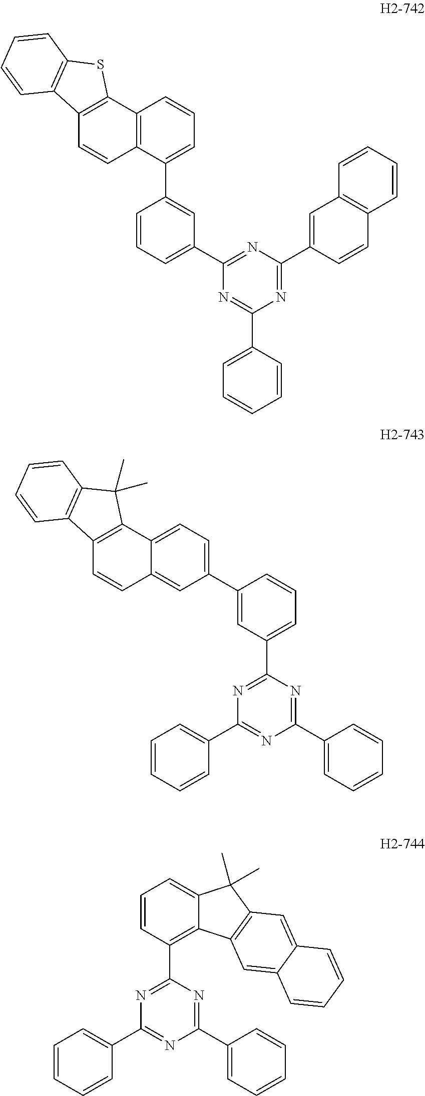 [SMILES: c1ccc(-c2nc(-c3cccc(-c4cccc5c4ccc4c6ccccc6sc54)c3)nc(-c3ccc4ccccc4c3)n2)cc1 ; CC1(C)c2ccccc2-c2ccc3cc(-c4cccc(-c5nc(-c6ccccc6)nc(-c6ccccc6)n5)c4)ccc3c21 ; CC1(C)c2cc3ccccc3cc2-c2c(-c3nc(-c4ccccc4)nc(-c4ccccc4)n3)cccc21]

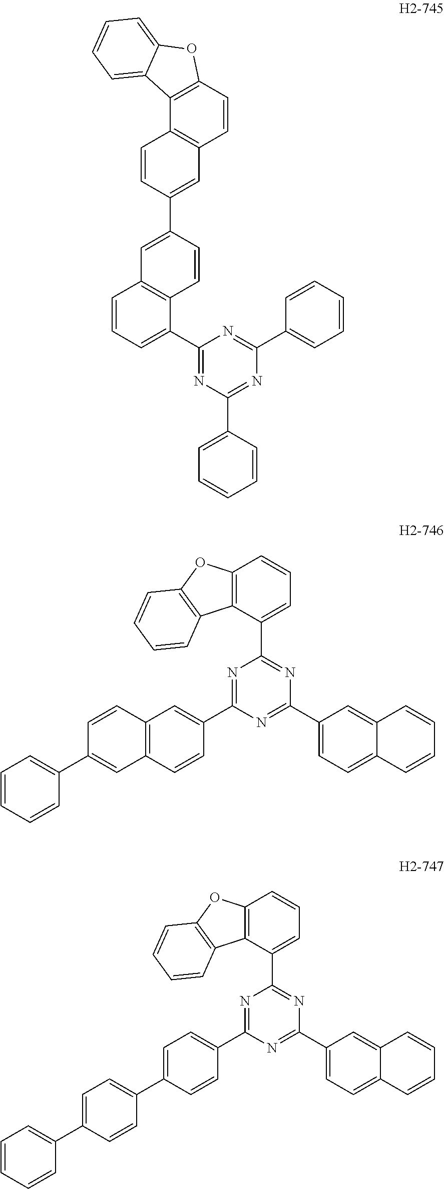 [SMILES: c1ccc(-c2nc(-c3ccccc3)nc(-c3cccc4cc(-c5ccc6c(ccc7oc8ccccc8c76)c5)ccc34)n2)cc1 ; c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccc6ccccc6c5)nc(-c5cccc6oc7ccccc7c56)n4)cc3)cc2)cc1]

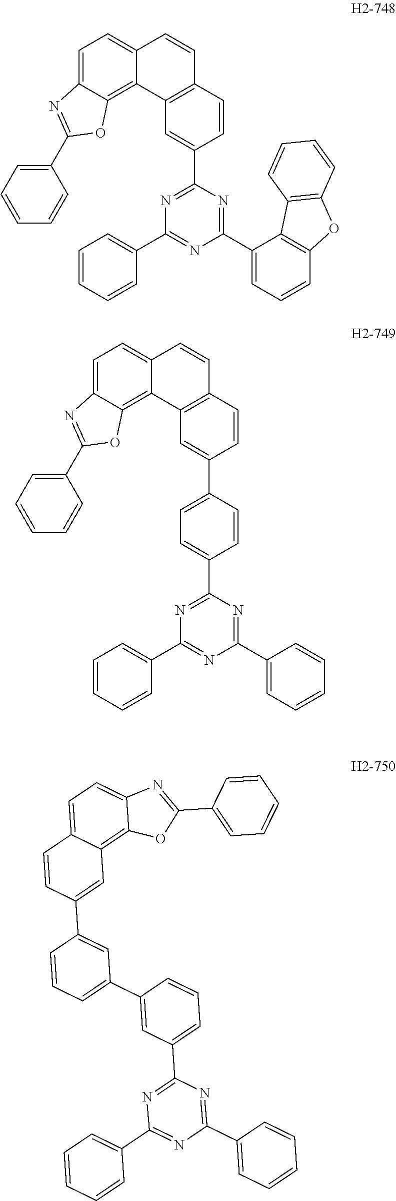

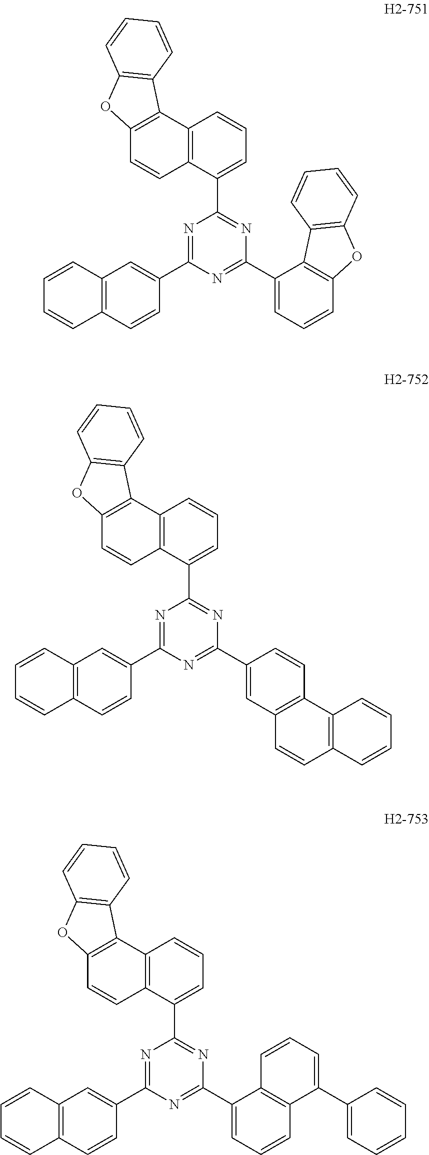 [SMILES: c1ccc2cc(-c3nc(-c4cccc5c4ccc4oc6ccccc6c45)nc(-c4cccc5oc6ccccc6c45)n3)ccc2c1 ; c1ccc2cc(-c3nc(-c4ccc5c(ccc6ccccc65)c4)nc(-c4cccc5c4ccc4oc6ccccc6c45)n3)ccc2c1 ; c1ccc(-c2cccc3c(-c4nc(-c5ccc6ccccc6c5)nc(-c5cccc6c5ccc5oc7ccccc7c56)n4)cccc23)cc1]

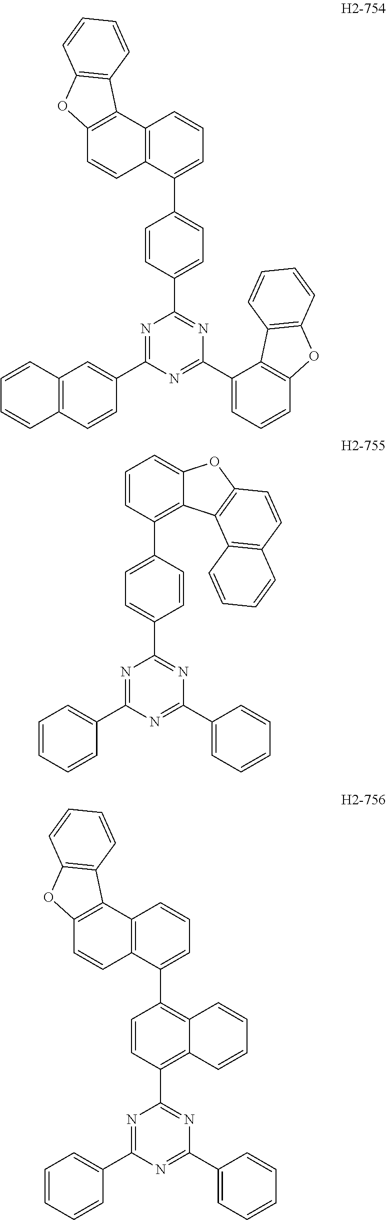

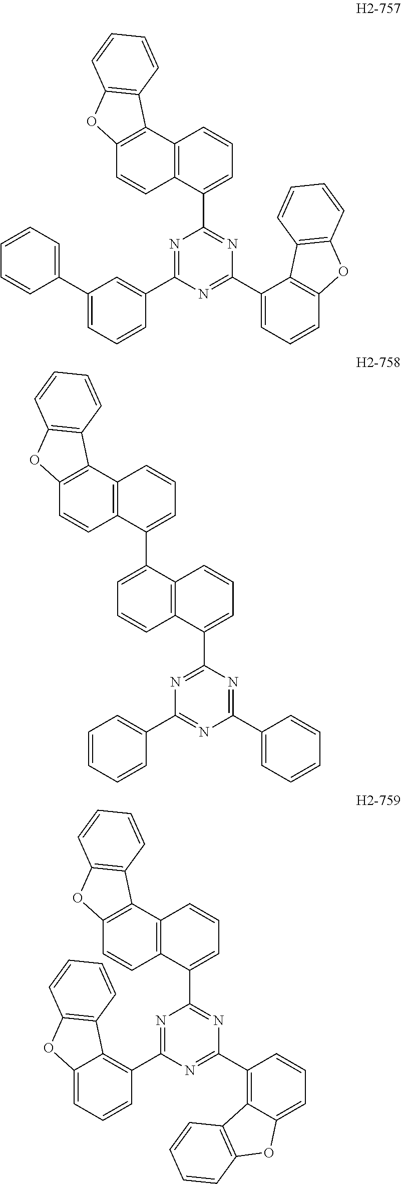

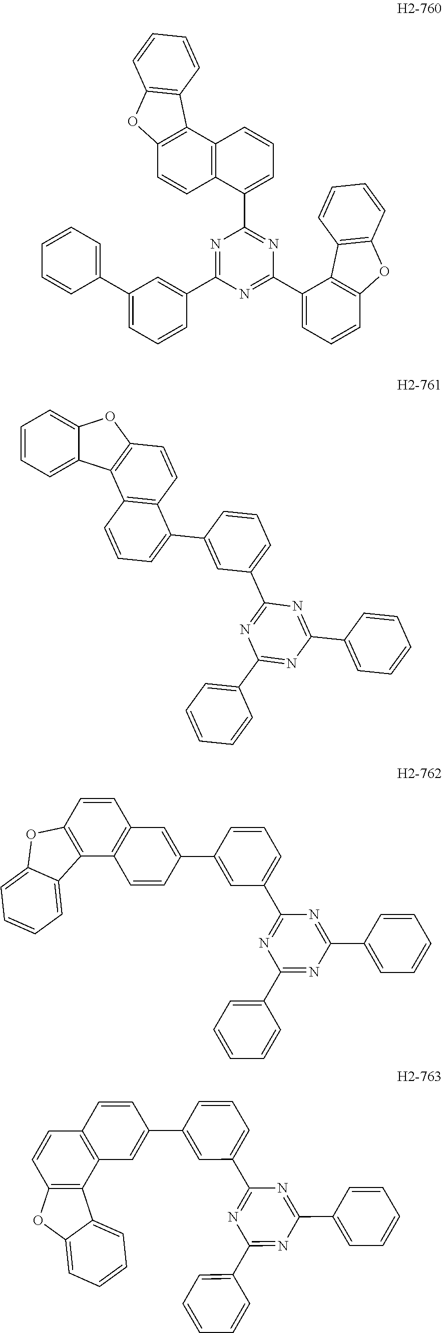

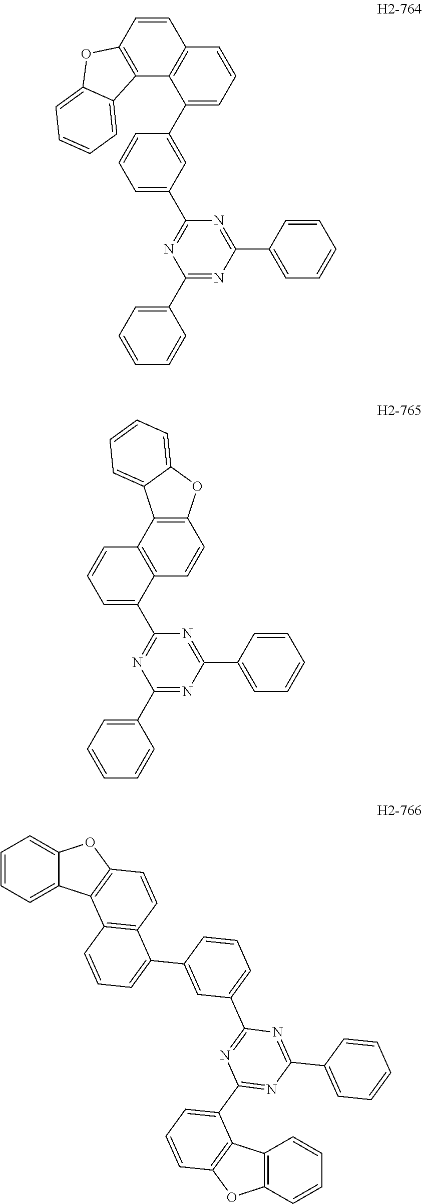 [SMILES: c1ccc(-c2nc(-c3ccccc3)nc(-c3cccc(-c4cccc5ccc6oc7ccccc7c6c45)c3)n2)cc1 ; c1ccc(-c2nc(-c3ccccc3)nc(-c3cccc4c3ccc3oc5ccccc5c34)n2)cc1 ; c1ccc(-c2nc(-c3cccc(-c4cccc5c4ccc4oc6ccccc6c45)c3)nc(-c3cccc4oc5ccccc5c34)n2)cc1]

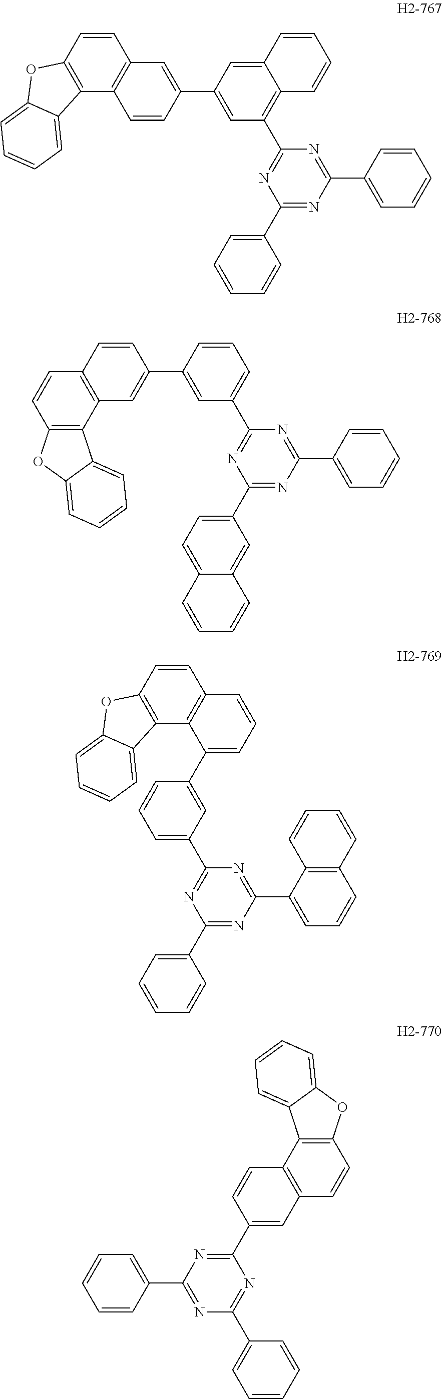

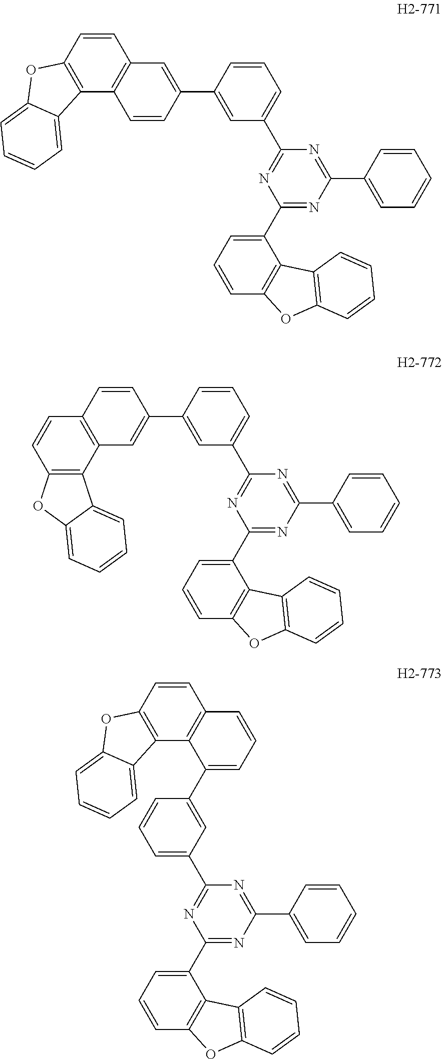 [SMILES: c1ccc(-c2nc(-c3cccc(-c4ccc5c(ccc6oc7ccccc7c65)c4)c3)nc(-c3cccc4oc5ccccc5c34)n2)cc1 ; c1ccc(-c2nc(-c3cccc(-c4ccc5ccc6oc7ccccc7c6c5c4)c3)nc(-c3cccc4oc5ccccc5c34)n2)cc1 ; c1ccc(-c2nc(-c3cccc(-c4cccc5ccc6oc7ccccc7c6c45)c3)nc(-c3cccc4oc5ccccc5c34)n2)cc1]

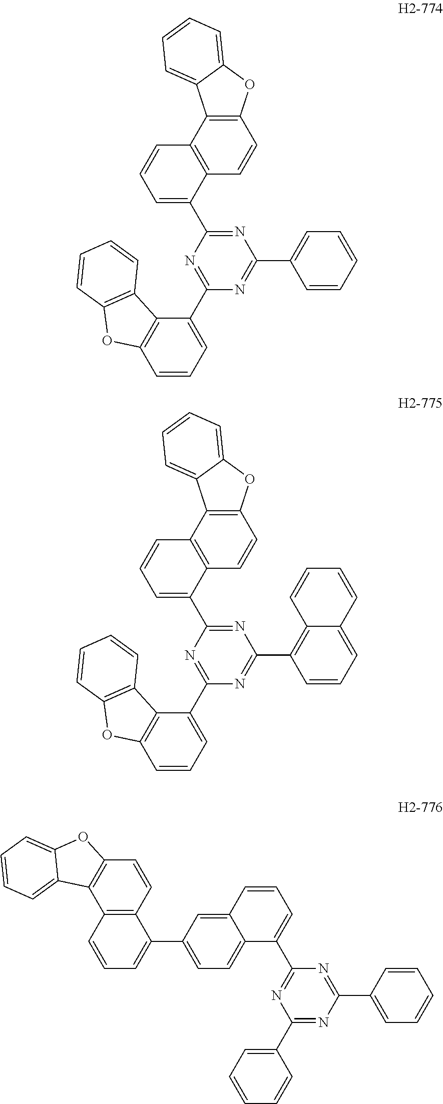

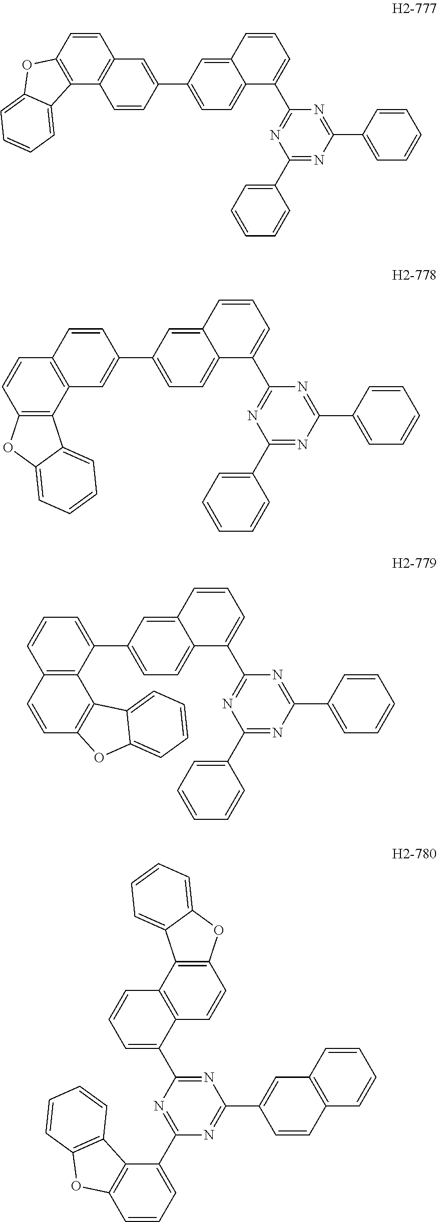 [SMILES: c1ccc(-c2nc(-c3ccccc3)nc(-c3cccc4cc(-c5ccc6c(ccc7oc8ccccc8c76)c5)ccc34)n2)cc1 ; c1ccc(-c2nc(-c3ccccc3)nc(-c3cccc4cc(-c5ccc6ccc7oc8ccccc8c7c6c5)ccc34)n2)cc1 ; c1ccc(-c2nc(-c3ccccc3)nc(-c3cccc4cc(-c5cccc6ccc7oc8ccccc8c7c56)ccc34)n2)cc1 ; c1ccc2cc(-c3nc(-c4cccc5c4ccc4oc6ccccc6c45)nc(-c4cccc5oc6ccccc6c45)n3)ccc2c1]

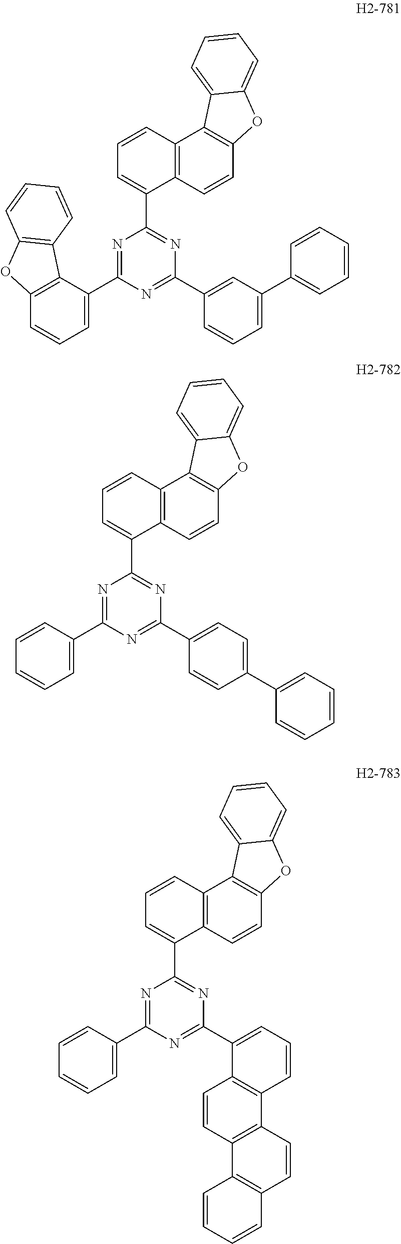

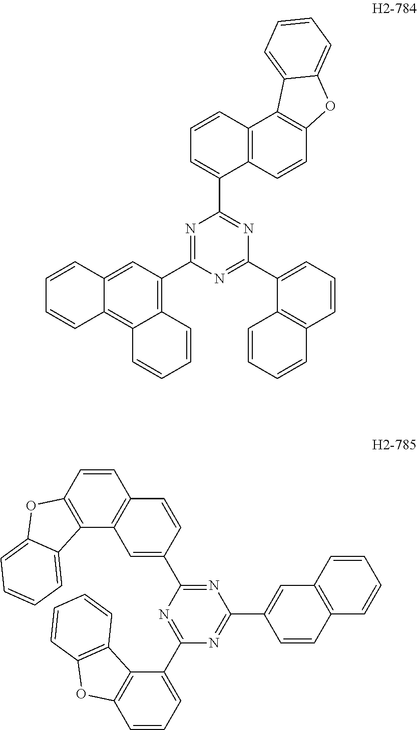 [SMILES: c1ccc2c(-c3nc(-c4cc5ccccc5c5ccccc45)nc(-c4cccc5c4ccc4oc6ccccc6c45)n3)cccc2c1 ; c1ccc2cc(-c3nc(-c4ccc5ccc6oc7ccccc7c6c5c4)nc(-c4cccc5oc6ccccc6c45)n3)ccc2c1]

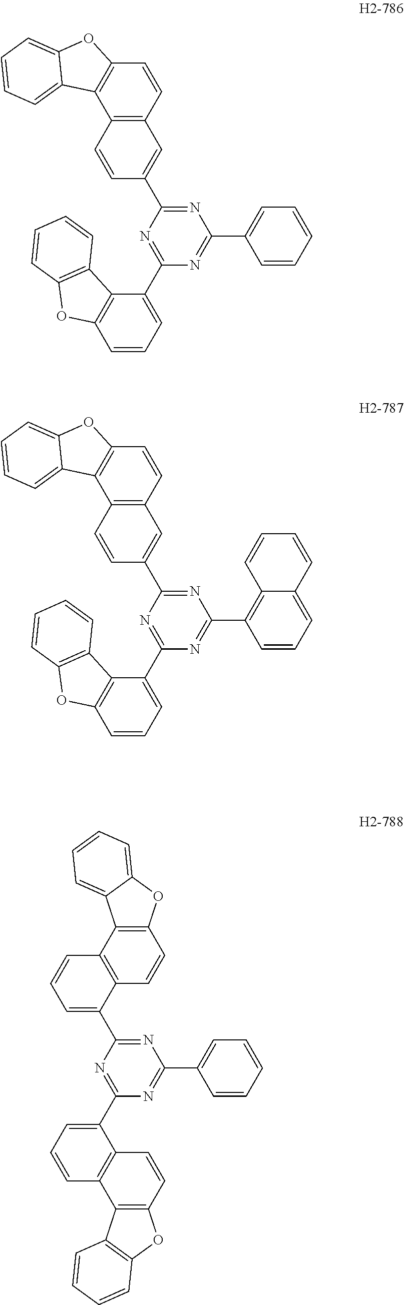 [SMILES: c1ccc(-c2nc(-c3ccc4c(ccc5oc6ccccc6c54)c3)nc(-c3cccc4oc5ccccc5c34)n2)cc1 ; c1ccc2c(-c3nc(-c4ccc5c(ccc6oc7ccccc7c65)c4)nc(-c4cccc5oc6ccccc6c45)n3)cccc2c1 ; c1ccc(-c2nc(-c3cccc4c3ccc3oc5ccccc5c34)nc(-c3cccc4c3ccc3oc5ccccc5c34)n2)cc1]

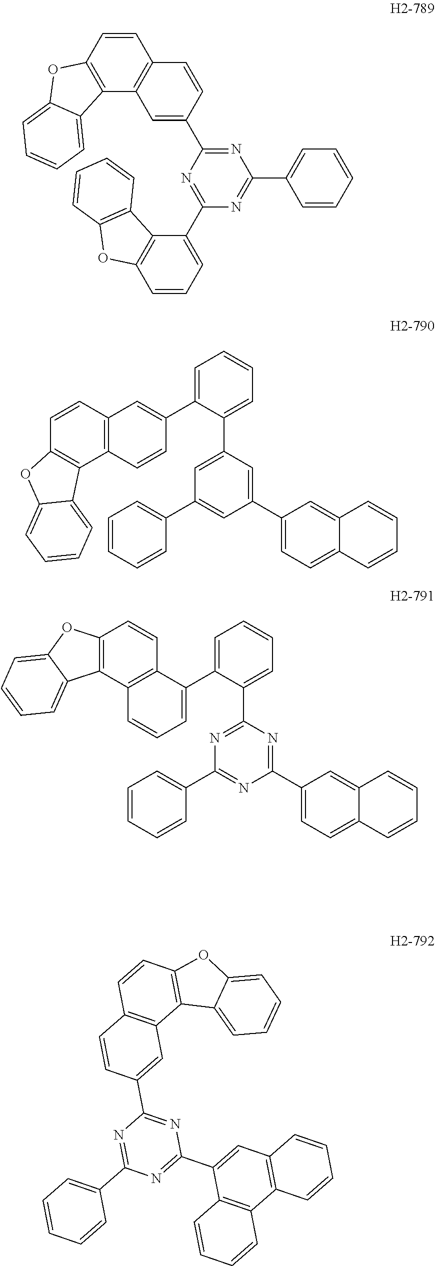 [SMILES: c1ccc(-c2nc(-c3ccc4ccc5oc6ccccc6c5c4c3)nc(-c3cccc4oc5ccccc5c34)n2)cc1 ; c1ccc(-c2cc(-c3ccc4ccccc4c3)cc(-c3ccccc3-c3ccc4c(ccc5oc6ccccc6c54)c3)c2)cc1 ; c1ccc(-c2nc(-c3ccc4ccccc4c3)nc(-c3ccccc3-c3cccc4c3ccc3oc5ccccc5c34)n2)cc1 ; c1ccc(-c2nc(-c3ccc4ccc5oc6ccccc6c5c4c3)nc(-c3cc4ccccc4c4ccccc34)n2)cc1]

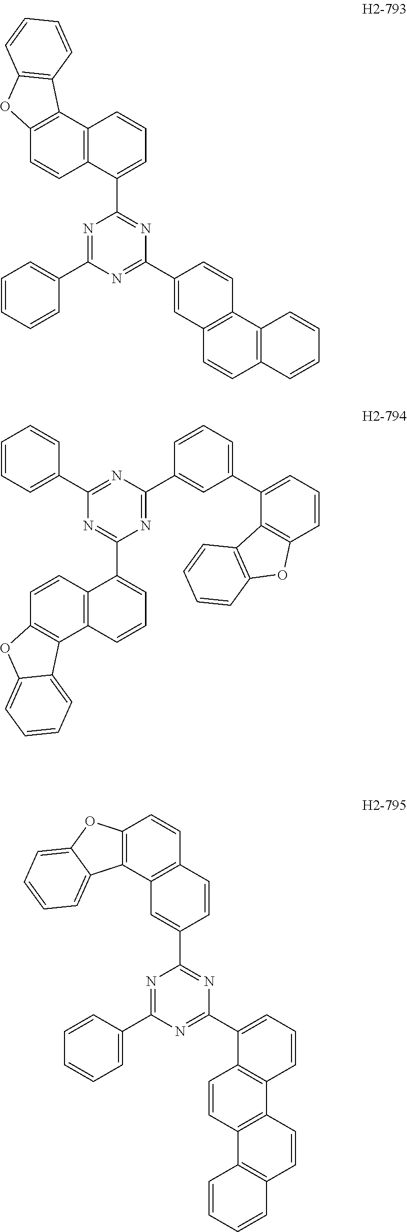 [SMILES: c1ccc(-c2nc(-c3ccc4c(ccc5ccccc54)c3)nc(-c3cccc4c3ccc3oc5ccccc5c34)n2)cc1 ; c1ccc(-c2nc(-c3cccc(-c4cccc5oc6ccccc6c45)c3)nc(-c3cccc4c3ccc3oc5ccccc5c34)n2)cc1 ; c1ccc(-c2nc(-c3ccc4ccc5oc6ccccc6c5c4c3)nc(-c3cccc4c3ccc3c5ccccc5ccc43)n2)cc1]

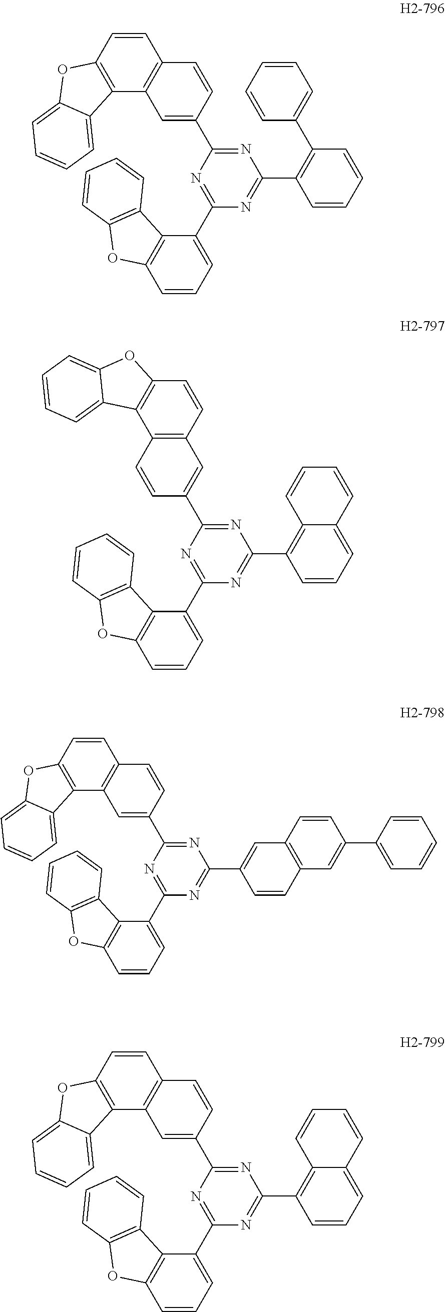 [SMILES: c1ccc(-c2ccccc2-c2nc(-c3ccc4ccc5oc6ccccc6c5c4c3)nc(-c3cccc4oc5ccccc5c34)n2)cc1 ; c1ccc2c(-c3nc(-c4ccc5c(ccc6oc7ccccc7c65)c4)nc(-c4cccc5oc6ccccc6c45)n3)cccc2c1 ; c1ccc(-c2ccc3cc(-c4nc(-c5ccc6ccc7oc8ccccc8c7c6c5)nc(-c5cccc6oc7ccccc7c56)n4)ccc3c2)cc1 ; c1ccc2c(-c3nc(-c4ccc5ccc6oc7ccccc7c6c5c4)nc(-c4cccc5oc6ccccc6c45)n3)cccc2c1]

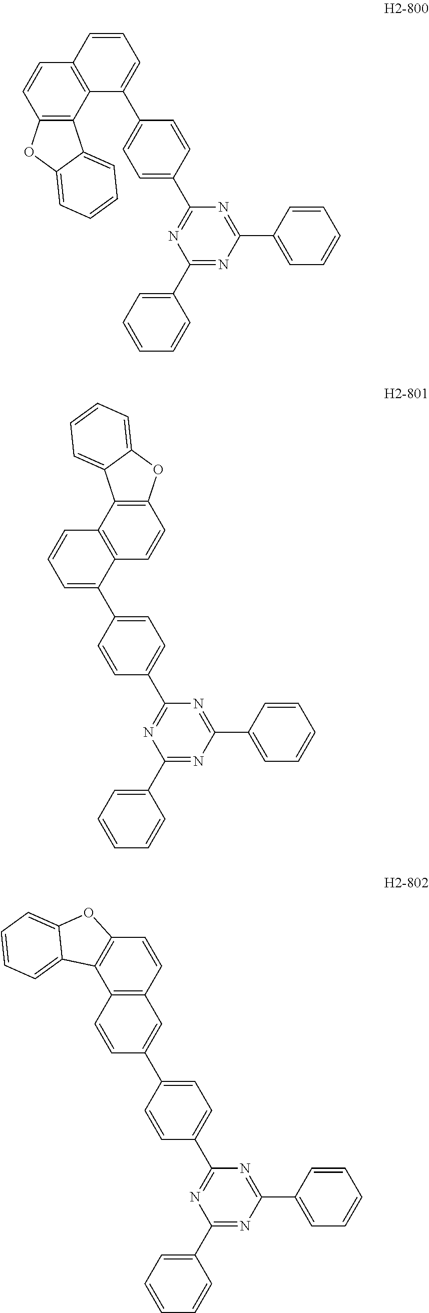 [SMILES: c1ccc(-c2nc(-c3ccccc3)nc(-c3ccc(-c4cccc5ccc6oc7ccccc7c6c45)cc3)n2)cc1 ; c1ccc(-c2nc(-c3ccccc3)nc(-c3ccc(-c4cccc5c4ccc4oc6ccccc6c45)cc3)n2)cc1 ; c1ccc(-c2nc(-c3ccccc3)nc(-c3ccc(-c4ccc5c(ccc6oc7ccccc7c65)c4)cc3)n2)cc1]

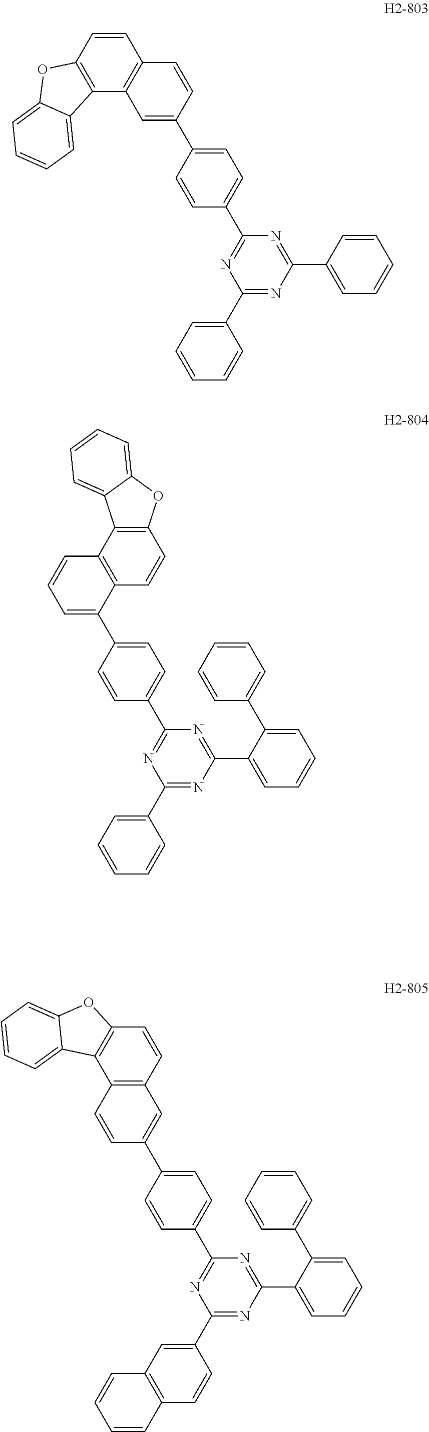 [SMILES: c1ccc(-c2nc(-c3ccccc3)nc(-c3ccc(-c4ccc5ccc6oc7ccccc7c6c5c4)cc3)n2)cc1 ; c1ccc(-c2nc(-c3ccc(-c4cccc5c4ccc4oc6ccccc6c45)cc3)nc(-c3ccccc3-c3ccccc3)n2)cc1 ; c1ccc(-c2ccccc2-c2nc(-c3ccc(-c4ccc5c(ccc6oc7ccccc7c65)c4)cc3)nc(-c3ccc4ccccc4c3)n2)cc1]

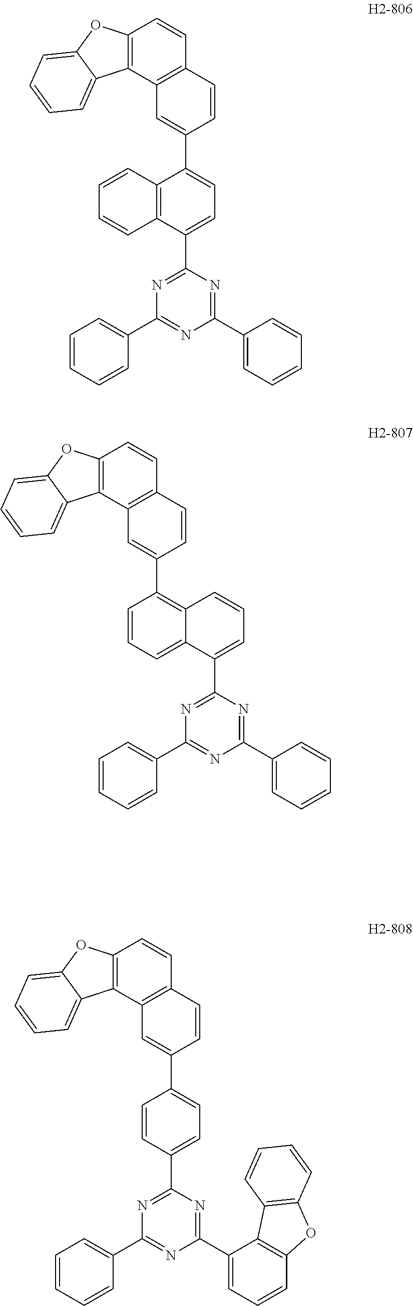 [SMILES: c1ccc(-c2nc(-c3ccccc3)nc(-c3ccc(-c4ccc5ccc6oc7ccccc7c6c5c4)c4ccccc34)n2)cc1 ; c1ccc(-c2nc(-c3ccccc3)nc(-c3cccc4c(-c5ccc6ccc7oc8ccccc8c7c6c5)cccc34)n2)cc1 ; c1ccc(-c2nc(-c3ccc(-c4ccc5ccc6oc7ccccc7c6c5c4)cc3)nc(-c3cccc4oc5ccccc5c34)n2)cc1]

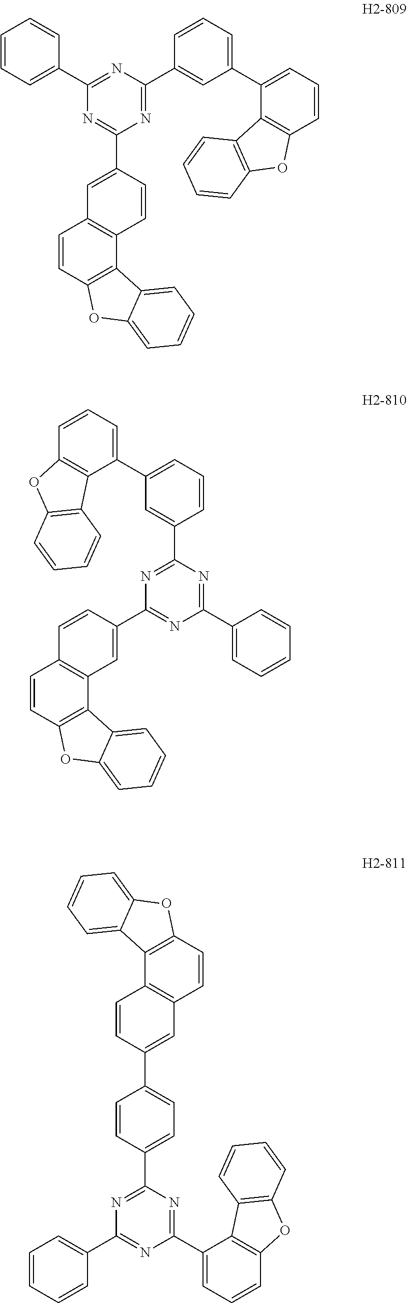 [SMILES: c1ccc(-c2nc(-c3cccc(-c4cccc5oc6ccccc6c45)c3)nc(-c3ccc4c(ccc5oc6ccccc6c54)c3)n2)cc1 ; c1ccc(-c2nc(-c3cccc(-c4cccc5oc6ccccc6c45)c3)nc(-c3ccc4ccc5oc6ccccc6c5c4c3)n2)cc1 ; c1ccc(-c2nc(-c3ccc(-c4ccc5c(ccc6oc7ccccc7c65)c4)cc3)nc(-c3cccc4oc5ccccc5c34)n2)cc1]

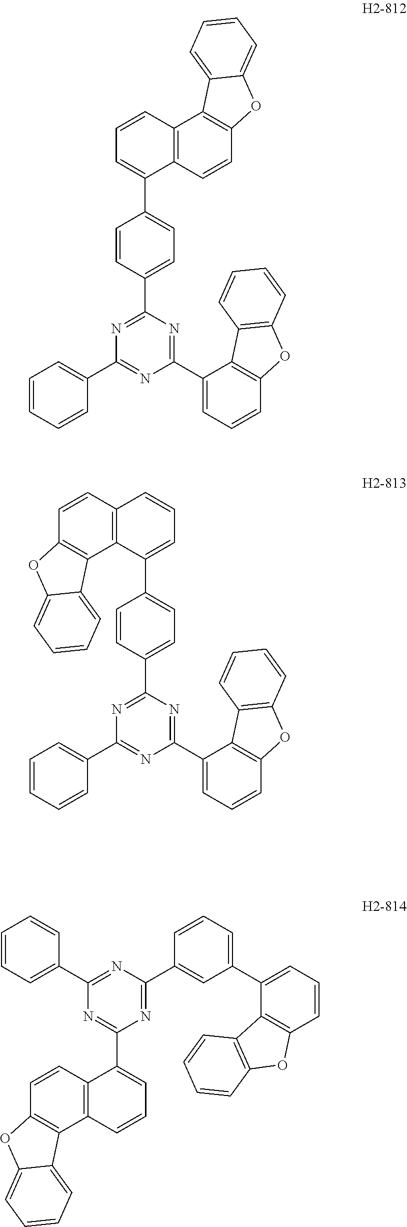 [SMILES: c1ccc(-c2nc(-c3ccc(-c4cccc5c4ccc4oc6ccccc6c45)cc3)nc(-c3cccc4oc5ccccc5c34)n2)cc1 ; c1ccc(-c2nc(-c3ccc(-c4cccc5ccc6oc7ccccc7c6c45)cc3)nc(-c3cccc4oc5ccccc5c34)n2)cc1 ; c1ccc(-c2nc(-c3cccc(-c4cccc5oc6ccccc6c45)c3)nc(-c3cccc4c3ccc3oc5ccccc5c34)n2)cc1]

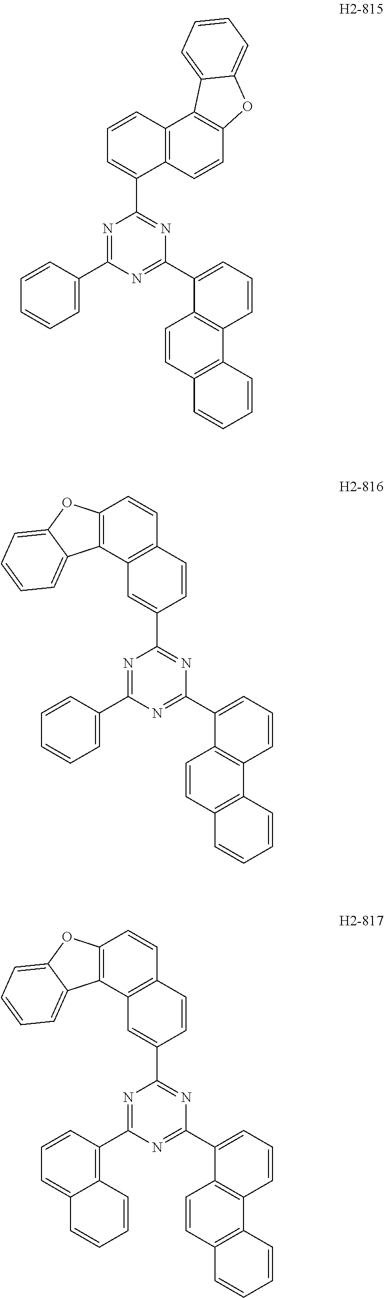 [SMILES: c1ccc(-c2nc(-c3cccc4c3ccc3ccccc34)nc(-c3cccc4c3ccc3oc5ccccc5c34)n2)cc1 ; c1ccc(-c2nc(-c3ccc4ccc5oc6ccccc6c5c4c3)nc(-c3cccc4c3ccc3ccccc34)n2)cc1 ; c1ccc2c(-c3nc(-c4ccc5ccc6oc7ccccc7c6c5c4)nc(-c4cccc5c4ccc4ccccc45)n3)cccc2c1]

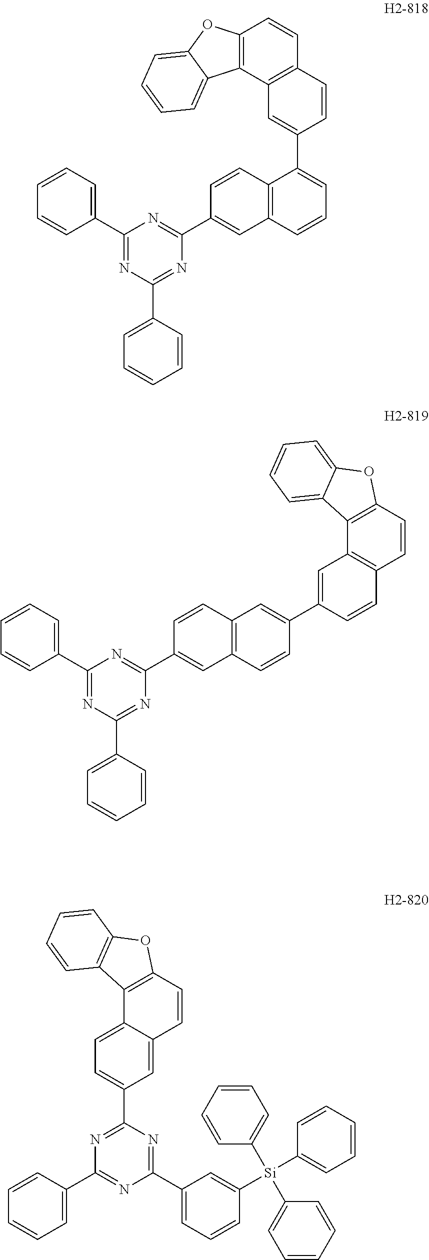 [SMILES: c1ccc(-c2nc(-c3ccccc3)nc(-c3ccc4c(-c5ccc6ccc7oc8ccccc8c7c6c5)cccc4c3)n2)cc1 ; c1ccc(-c2nc(-c3ccccc3)nc(-c3ccc4cc(-c5ccc6ccc7oc8ccccc8c7c6c5)ccc4c3)n2)cc1 ; c1ccc(-c2nc(-c3cccc([Si](c4ccccc4)(c4ccccc4)c4ccccc4)c3)nc(-c3ccc4c(ccc5oc6ccccc6c54)c3)n2)cc1]

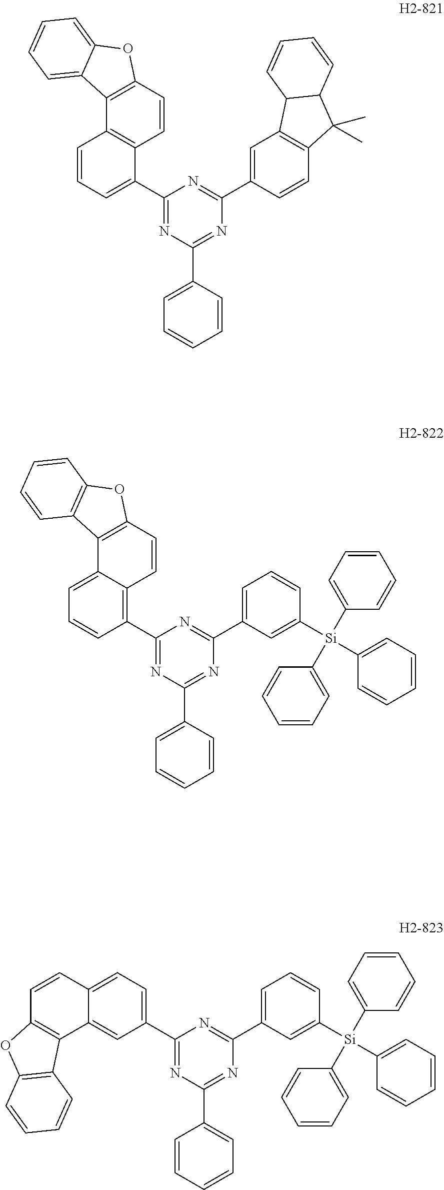 [SMILES: CC1(C)c2ccc(-c3nc(-c4ccccc4)nc(-c4cccc5c4ccc4oc6ccccc6c45)n3)cc2C2C=CC=CC21 ; c1ccc(-c2nc(-c3cccc([Si](c4ccccc4)(c4ccccc4)c4ccccc4)c3)nc(-c3cccc4c3ccc3oc5ccccc5c34)n2)cc1 ; c1ccc(-c2nc(-c3cccc([Si](c4ccccc4)(c4ccccc4)c4ccccc4)c3)nc(-c3ccc4ccc5oc6ccccc6c5c4c3)n2)cc1]

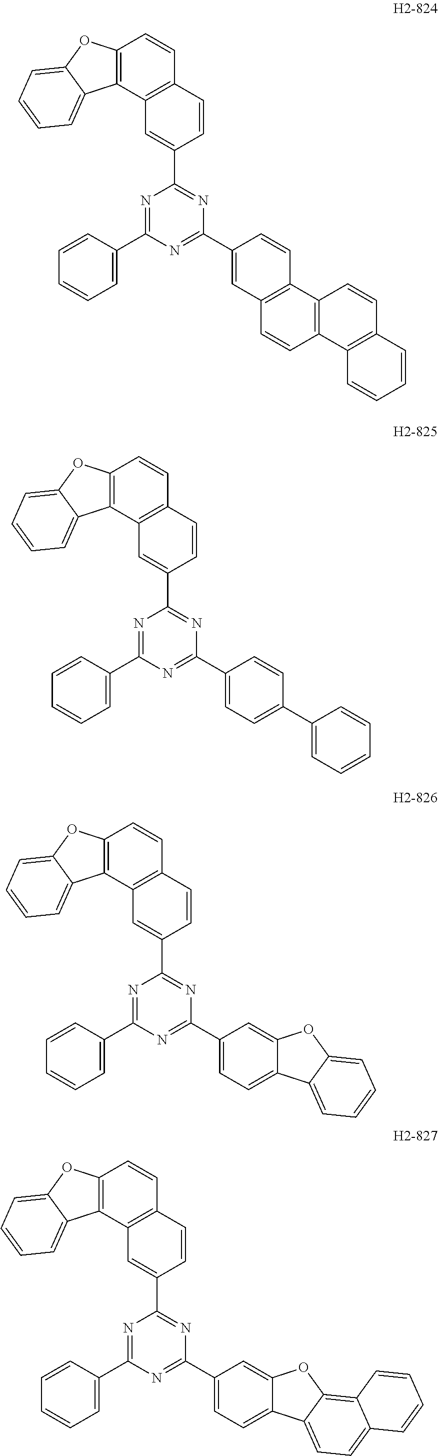 [SMILES: c1ccc(-c2nc(-c3ccc4c(ccc5c6ccccc6ccc45)c3)nc(-c3ccc4ccc5oc6ccccc6c5c4c3)n2)cc1 ; c1ccc(-c2ccc(-c3nc(-c4ccccc4)nc(-c4ccc5ccc6oc7ccccc7c6c5c4)n3)cc2)cc1 ; c1ccc(-c2nc(-c3ccc4c(c3)oc3ccccc34)nc(-c3ccc4ccc5oc6ccccc6c5c4c3)n2)cc1 ; c1ccc(-c2nc(-c3ccc4c(c3)oc3c5ccccc5ccc43)nc(-c3ccc4ccc5oc6ccccc6c5c4c3)n2)cc1]

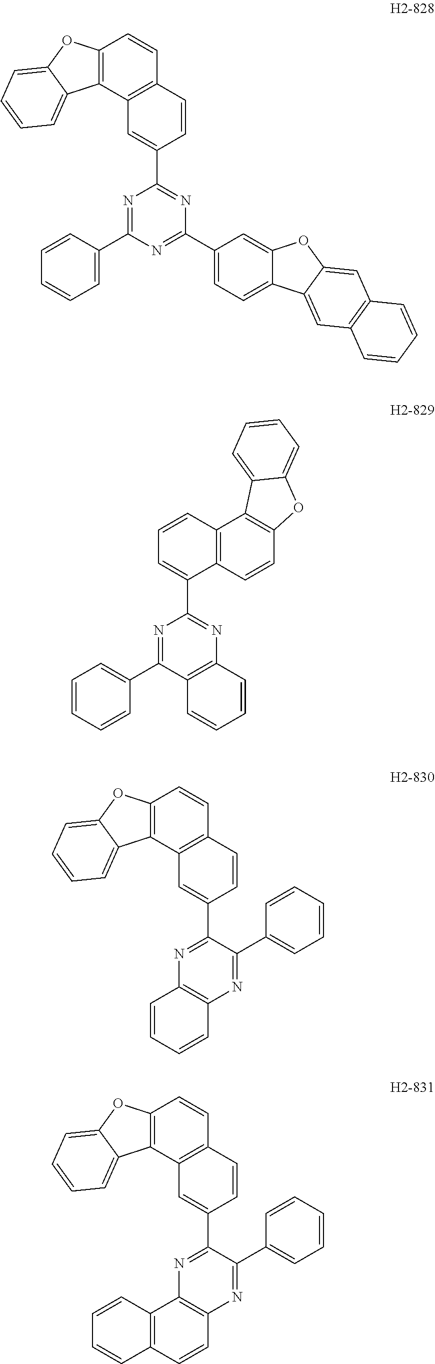

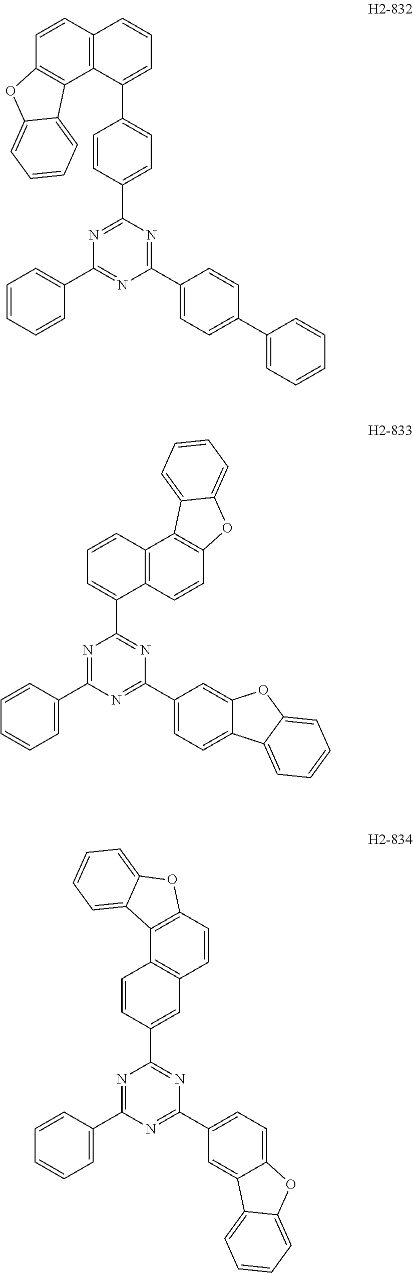 [SMILES: c1ccc(-c2ccc(-c3nc(-c4ccccc4)nc(-c4ccc(-c5cccc6ccc7oc8ccccc8c7c56)cc4)n3)cc2)cc1 ; c1ccc(-c2nc(-c3ccc4c(c3)oc3ccccc34)nc(-c3cccc4c3ccc3oc5ccccc5c34)n2)cc1 ; c1ccc(-c2nc(-c3ccc4c(ccc5oc6ccccc6c54)c3)nc(-c3ccc4oc5ccccc5c4c3)n2)cc1]

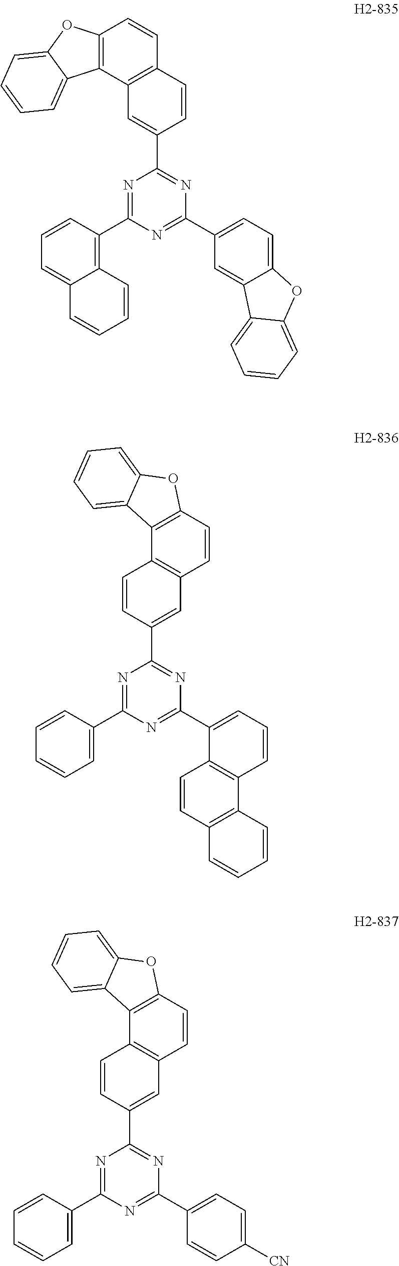 [SMILES: c1ccc2c(-c3nc(-c4ccc5oc6ccccc6c5c4)nc(-c4ccc5ccc6oc7ccccc7c6c5c4)n3)cccc2c1 ; c1ccc(-c2nc(-c3ccc4c(ccc5oc6ccccc6c54)c3)nc(-c3cccc4c3ccc3ccccc34)n2)cc1 ; N#Cc1ccc(-c2nc(-c3ccccc3)nc(-c3ccc4c(ccc5oc6ccccc6c54)c3)n2)cc1]

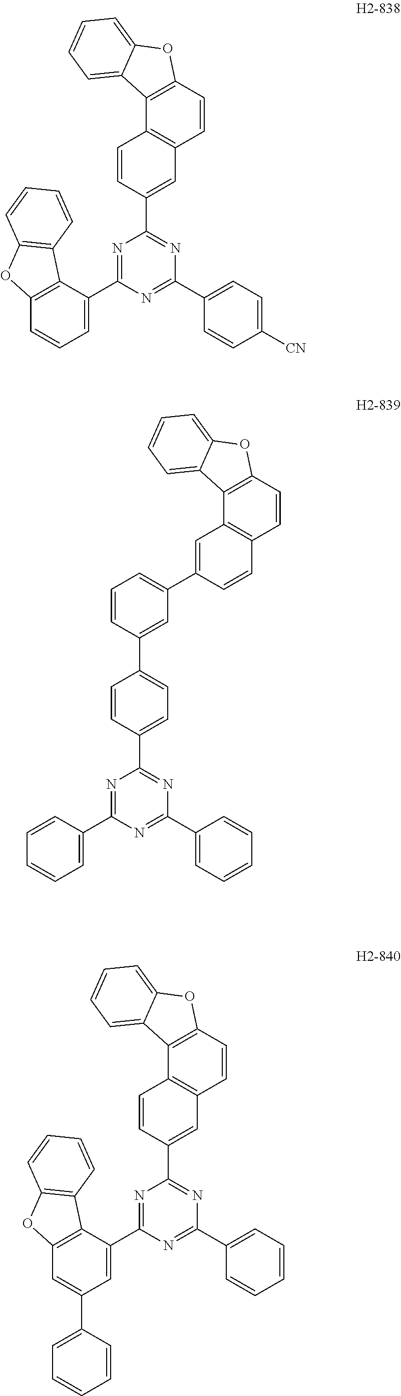 [SMILES: N#Cc1ccc(-c2nc(-c3ccc4c(ccc5oc6ccccc6c54)c3)nc(-c3cccc4oc5ccccc5c34)n2)cc1 ; c1ccc(-c2nc(-c3ccccc3)nc(-c3ccc(-c4cccc(-c5ccc6ccc7oc8ccccc8c7c6c5)c4)cc3)n2)cc1 ; c1ccc(-c2cc(-c3nc(-c4ccccc4)nc(-c4ccc5c(ccc6oc7ccccc7c65)c4)n3)c3c(c2)oc2ccccc23)cc1]

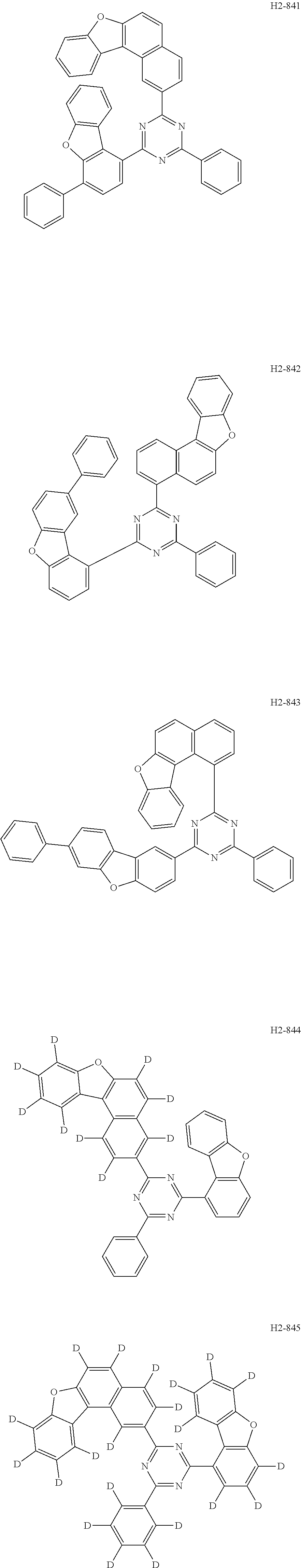

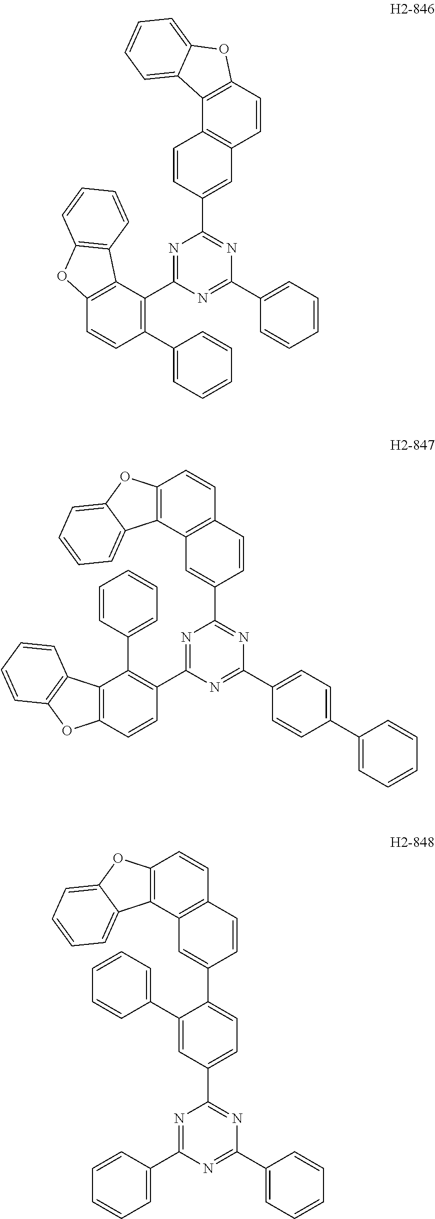 [SMILES: c1ccc(-c2nc(-c3ccc4c(ccc5oc6ccccc6c54)c3)nc(-c3c(-c4ccccc4)ccc4oc5ccccc5c34)n2)cc1 ; c1ccc(-c2ccc(-c3nc(-c4ccc5ccc6oc7ccccc7c6c5c4)nc(-c4ccc5oc6ccccc6c5c4-c4ccccc4)n3)cc2)cc1 ; c1ccc(-c2nc(-c3ccccc3)nc(-c3ccc(-c4ccc5ccc6oc7ccccc7c6c5c4)c(-c4ccccc4)c3)n2)cc1]

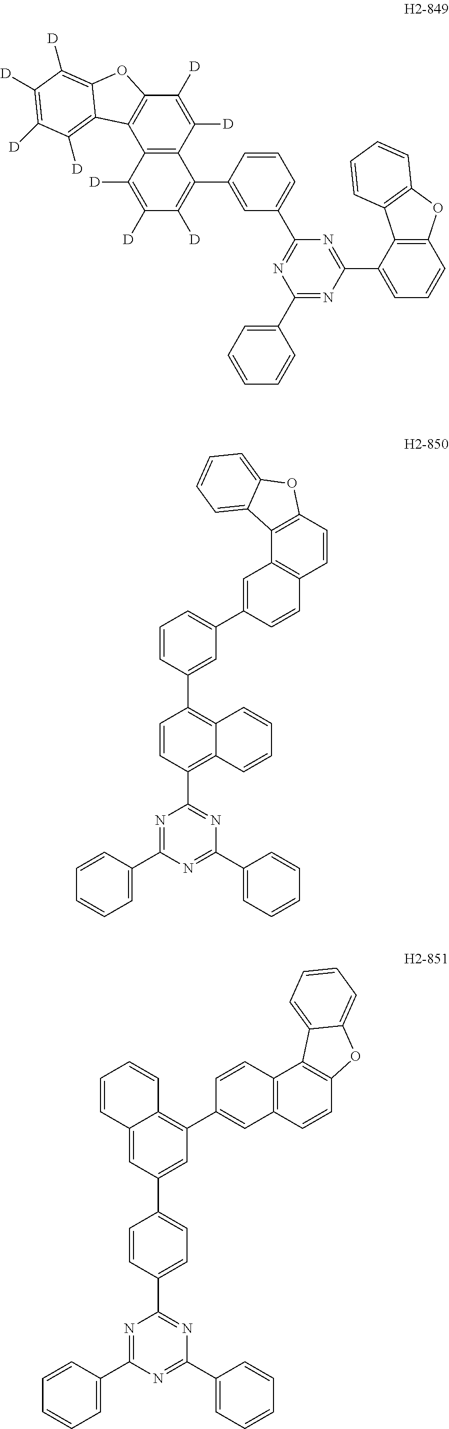 [SMILES: [2H]c1c([2H])c([2H])c2c(oc3c([2H])c([2H])c4c(-c5cccc(-c6nc(-c7ccccc7)nc(-c7cccc8oc9ccccc9c78)n6)c5)c([2H])c([2H])c([2H])c4c32)c1[2H] ; c1ccc(-c2nc(-c3ccccc3)nc(-c3ccc(-c4cccc(-c5ccc6ccc7oc8ccccc8c7c6c5)c4)c4ccccc34)n2)cc1 ; c1ccc(-c2nc(-c3ccccc3)nc(-c3ccc(-c4cc(-c5ccc6c(ccc7oc8ccccc8c76)c5)c5ccccc5c4)cc3)n2)cc1]

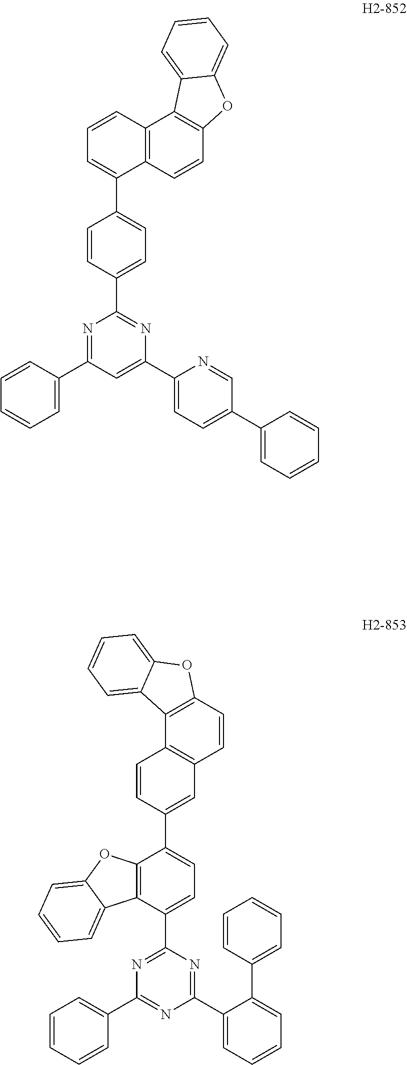 [SMILES: c1ccc(-c2ccc(-c3cc(-c4ccccc4)nc(-c4ccc(-c5cccc6c5ccc5oc7ccccc7c56)cc4)n3)nc2)cc1 ; c1ccc(-c2nc(-c3ccccc3-c3ccccc3)nc(-c3ccc(-c4ccc5c(ccc6oc7ccccc7c65)c4)c4oc5ccccc5c34)n2)cc1]

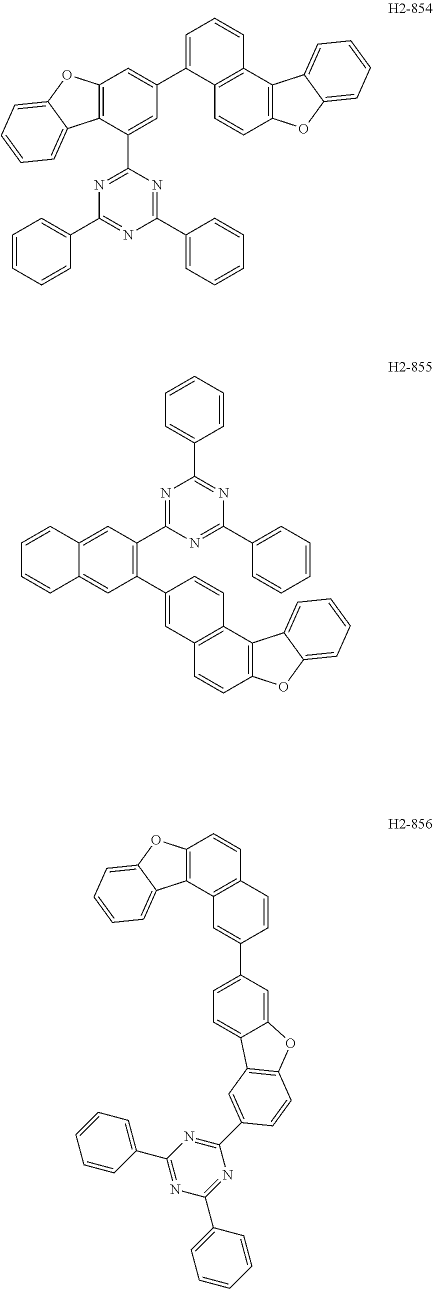 [SMILES: c1ccc(-c2nc(-c3ccccc3)nc(-c3cc(-c4cccc5c4ccc4oc6ccccc6c45)cc4oc5ccccc5c34)n2)cc1 ; c1ccc(-c2nc(-c3ccccc3)nc(-c3cc4ccccc4cc3-c3ccc4c(ccc5oc6ccccc6c54)c3)n2)cc1 ; c1ccc(-c2nc(-c3ccccc3)nc(-c3ccc4oc5cc(-c6ccc7ccc8oc9ccccc9c8c7c6)ccc5c4c3)n2)cc1]

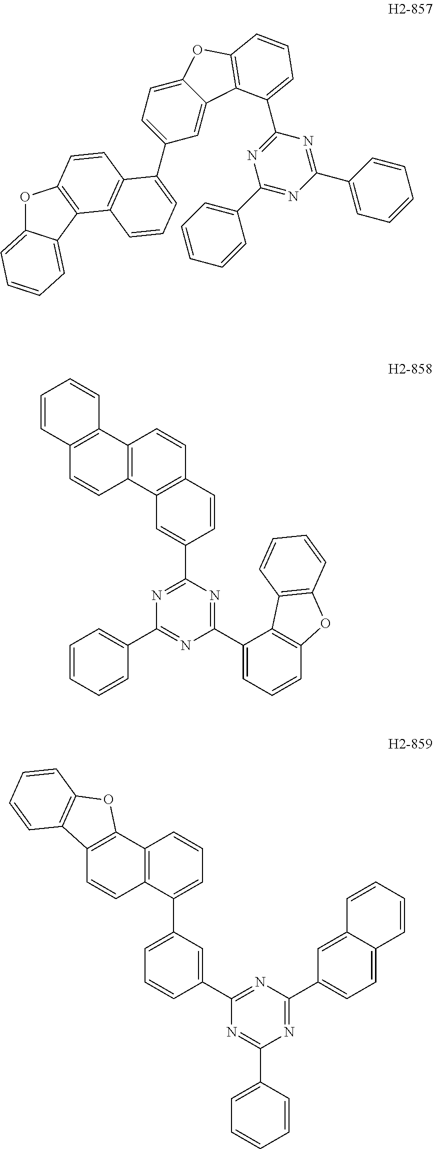 [SMILES: c1ccc(-c2nc(-c3ccccc3)nc(-c3cccc4oc5ccc(-c6cccc7c6ccc6oc8ccccc8c67)cc5c34)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3cccc(-c4cccc5c4ccc4c6ccccc6oc54)c3)nc(-c3ccc4ccccc4c3)n2)cc1]

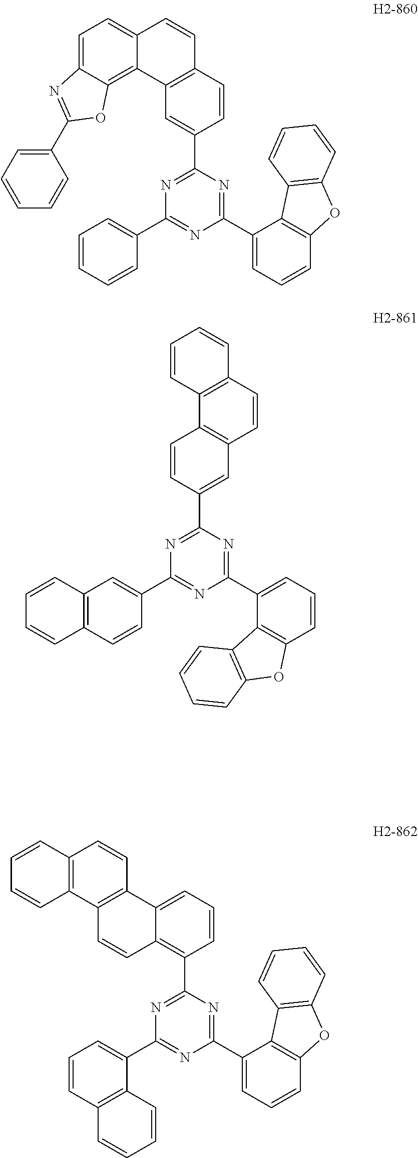 [SMILES: c1ccc(-c2nc(-c3ccc4ccc5ccc6nc(-c7ccccc7)oc6c5c4c3)nc(-c3cccc4oc5ccccc5c34)n2)cc1 ; c1ccc2cc(-c3nc(-c4ccc5c(ccc6ccccc65)c4)nc(-c4cccc5oc6ccccc6c45)n3)ccc2c1 ; c1ccc2c(-c3nc(-c4cccc5c4ccc4c6ccccc6ccc54)nc(-c4cccc5oc6ccccc6c45)n3)cccc2c1]

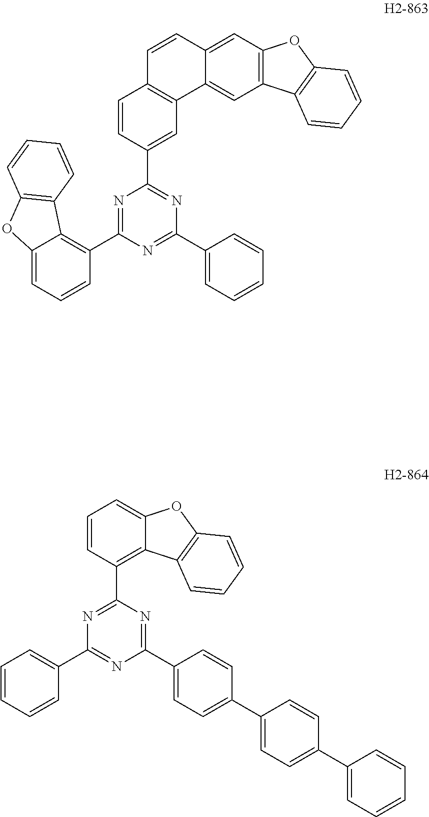 [SMILES: c1ccc(-c2nc(-c3ccc4ccc5cc6oc7ccccc7c6cc5c4c3)nc(-c3cccc4oc5ccccc5c34)n2)cc1 ; c1ccc(-c2ccc(-c3ccc(-c4nc(-c5ccccc5)nc(-c5cccc6oc7ccccc7c56)n4)cc3)cc2)cc1]

The combination of at least one of compounds H1-1 to H1-320 and at least one of compounds H2-1 to H2-864 may be used in an organic electroluminescent device. For example, the combination of any one of compounds H1-1 to H1-320, any one of compounds H2-1 to H2-864, and any one of compounds H1-1 to H1-320 and compounds H2-1 to H2-864 may be used in an organic electroluminescent device as the three types of host materials.

The compounds represented by formulas 1 to 4 according to the present disclosure may be produced by synthetic methods known to one skilled in the art. For example, the compounds represented by formulas 1 to 4 of the present disclosure may be produced by referring to Korean Patent Application Laid-Open Nos. 2020-0007644 (published on Jan. 22, 2020), 2018-0099487 (published on Sep. 5, 2018), 2020-0092879 (published on Aug. 4, 2020), 2020-0011884 (published on Feb. 4, 2020), and 2018-0099510 (published on Sep. 5, 2018), Korean Patent Publication No. 1545774 (published on Aug. 19, 2015), etc., and the compound represented by formula 2 of the present disclosure may be produced by referring to the following reaction schemes 1 and 2, but is not limited thereto.

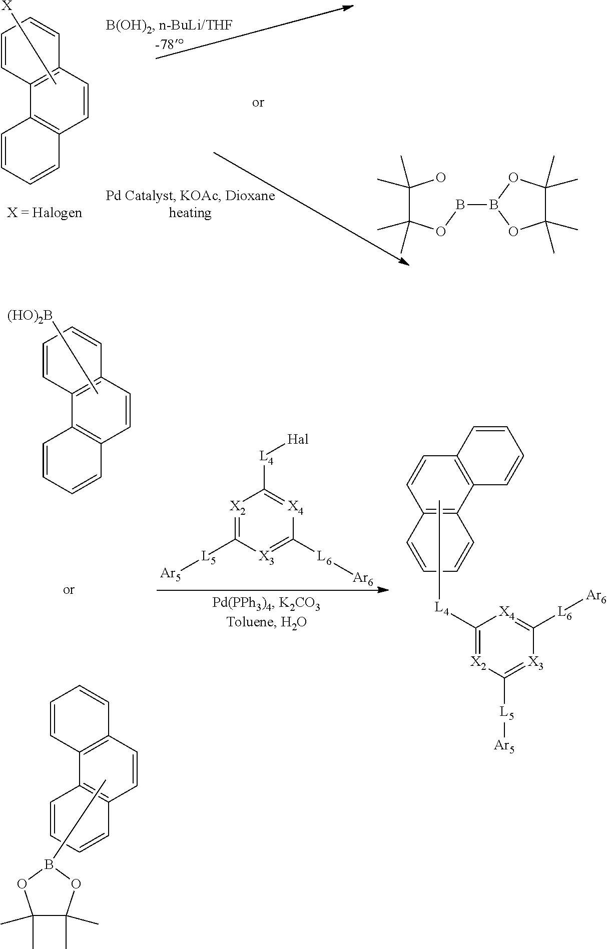

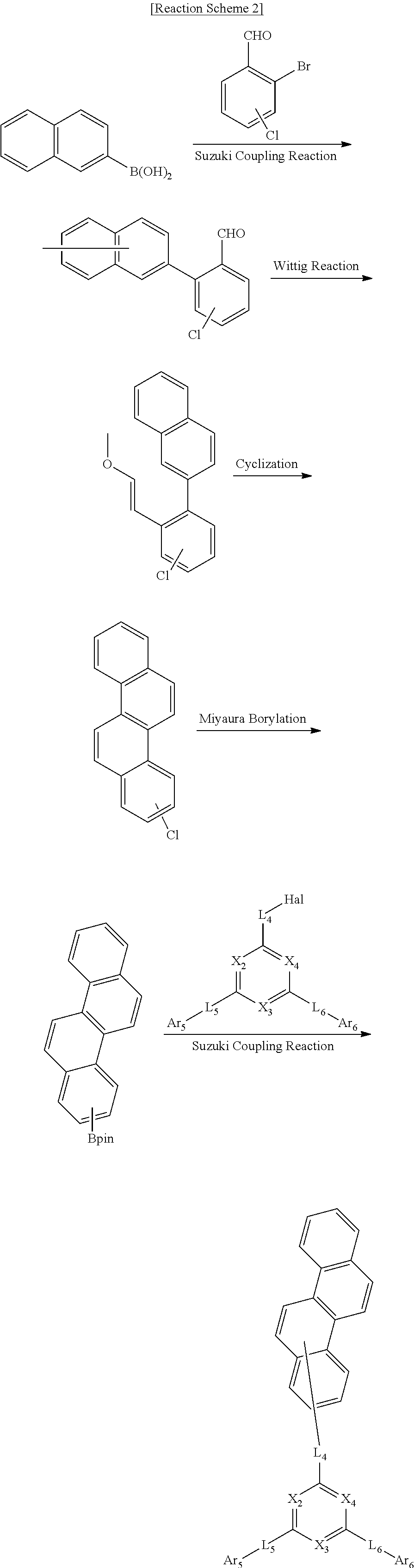

In reaction schemes 1 and 2, Hal represents a halogen, and X₂ to X₄, L₄ to L₆, Ar₅, and Ar₆ are as defined in formula 2.

Although illustrative synthesis examples of the compound represented by formula 2 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, a Phosphine-mediated reductive cyclization reaction, etc., and the reactions above proceed even when substituents, which are defined in formula 2 but are not specified in the specific synthesis examples, are bonded.

An organic electroluminescent device according to the present disclosure has a first electrode, a second electrode, and at least one organic layer between the first electrode and the second electrode.

One of the first and second electrodes may be an anode, and the other may be a cathode. The organic layer comprises a light-emitting layer and may further comprise at least one layer selected from the group consisting of a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Herein, the second electrode may be a transflective electrode or a reflective electrode, and may be a top emission type, a bottom emission type, or a both-sides emission type, depending on the material. In addition, the hole injection layer may be further doped with a p-dopant(s), and the electron injection layer may be further doped with an n-dopant(s).

An organic electroluminescent device according to the present disclosure comprises an anode, a cathode, and at least one organic layer between the anode and the cathode. The organic layer may comprise at least three types of the organic electroluminescent materials comprising the compound represented by formula 1 as the first organic electroluminescent material, the compound represented by any one of formulas 2 to 4 as the second organic electroluminescent material, and the compound represented by any one of formulas 1 to 4 as the third organic electroluminescent material. According to one embodiment, the organic electroluminescent device according to the present disclosure comprises an anode, a cathode, and at least one light-emitting layer between the anode and the cathode, wherein the light-emitting layer may comprise the compound represented by formula 1 and the compound represented by formula 2.

The light-emitting layer comprises a host and a dopant, and the host comprises a plurality of host materials. The compound represented by formula 1 may be comprised as the first host compound of the plurality of host materials. The compound represented by formula 2 may be comprised as the second host compound of the plurality of host materials. The compound represented by formula 1 or 2 while being different from the first and second host compounds may be comprised as the third host compound of the plurality of host materials. Herein, the first host material of the plurality of host materials of the present disclosure may be from about 5 wt % to about 90 wt %, preferably from about 10 wt % to about 90 wt %, more preferably from about 10 wt % to about 80 wt %, even more preferably from about 15 wt % to about 70 wt %, even more preferably from about 30 wt % to about 70 wt %, even more preferably from about 20 wt % to about 60 wt %, even more preferably from about 30 wt % to about 60 wt %. The second host material of the plurality of host materials of the present disclosure may be from about 5 wt % to about 90 wt %, preferably from about 10 wt % to about 90 wt %, more preferably from about 10 wt % to about 80 wt %, even more preferably from about 15 wt % to about 70 wt %, even more preferably from about 30 wt % to about 70 wt %, even more preferably from about 20 wt % to about 60 wt %, even more preferably from about 30 wt % to about 60 wt %. The third host material of the plurality of host materials of the present disclosure may be about 5 wt % to about 90 wt %, preferably about 10 wt % to about 90 wt %, more preferably about 10 wt % to about 80 wt %, even more preferably from about 15 wt % to about 70 wt %, even more preferably from about 30 wt % to about 70 wt %, even more preferably from about 20 wt % to about 60 wt %, even more preferably from about 30 wt % to about 60 wt %. For example, the plurality of host materials may comprise about 5 wt % to about 70 wt % of the first host material, about 5 wt % to about 70 wt % of the second host material, and about 10 wt % to about 90 wt % of the third host material.

According to one embodiment, any two of the first, second and third host materials of the present disclosure may have a difference in deposition temperature of about 0° C. to about 20° C. at 10⁻³ torr or less.

According to one embodiment, the maximum emission wavelength of the plurality of host materials of the present disclosure may be shifted by about 20 nm or more from the maximum emission wavelength of each of the first, second and third host materials.

In the present disclosure, the light-emitting layer is a layer from which light is emitted, and may be a single layer or a multi-layer of which two or more layers are stacked. All of the first, second and third host materials may be included in one layer; any two of the first, second and third host materials may be included in one layer; or the first, second and third host materials may be included in respective different light-emitting layers. According to one embodiment of the present disclosure, the doping concentration of the dopant compound with respect to the host compound(s) in the light-emitting layer may be less than about 20 wt %.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device of the present disclosure may further comprise an amine-based compound besides the plurality of host materials of the present disclosure as at least one of a hole injection material, a hole transport material, a hole auxiliary material, a light-emitting material, a light-emitting auxiliary material, and an electron blocking material. Further, according to one embodiment of the present disclosure, the organic electroluminescent device of the present disclosure may further comprise an azine-based compound besides the plurality of host materials of the present disclosure as at least one of an electron transport material, an electron injection material, an electron buffer material, and a hole blocking material.

The dopant comprised in the organic electroluminescent device of the present disclosure may be at least one phosphorescent or fluorescent dopant, and preferably a phosphorescent dopant. The phosphorescent dopant material applied to the organic electroluminescent device of the present disclosure is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may comprise a compound represented by the following formula 101, but is not limited thereto.

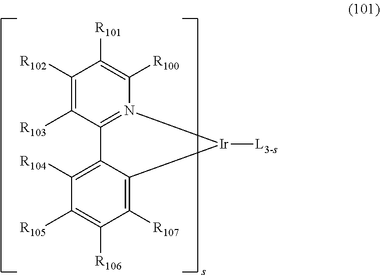

In formula 101, L is selected from the group consisting of the following structures 1 to 3:

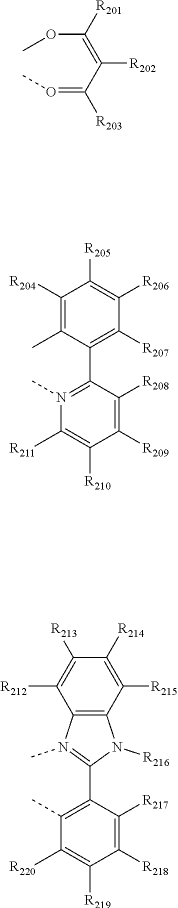

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, isoquinoline, benzofuropyridine, benzothienopyridine, indenopyridine, benzofuroquinoline, benzothienoquinoline, or indenoquinoline ring, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine or benzothienopyridine ring, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a ring(s); and
s represents an integer of 1 to 3.

The specific examples of the dopant compound are as follows, but are not limited thereto.

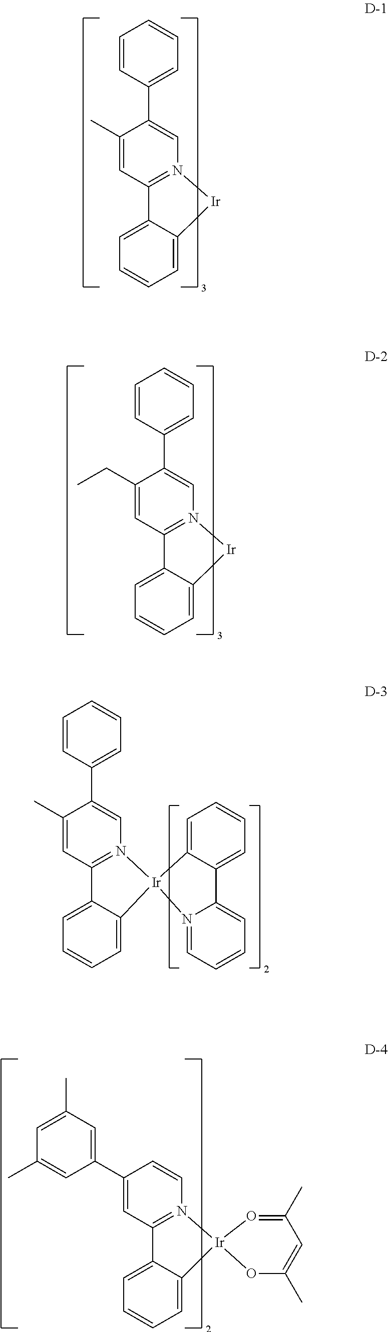

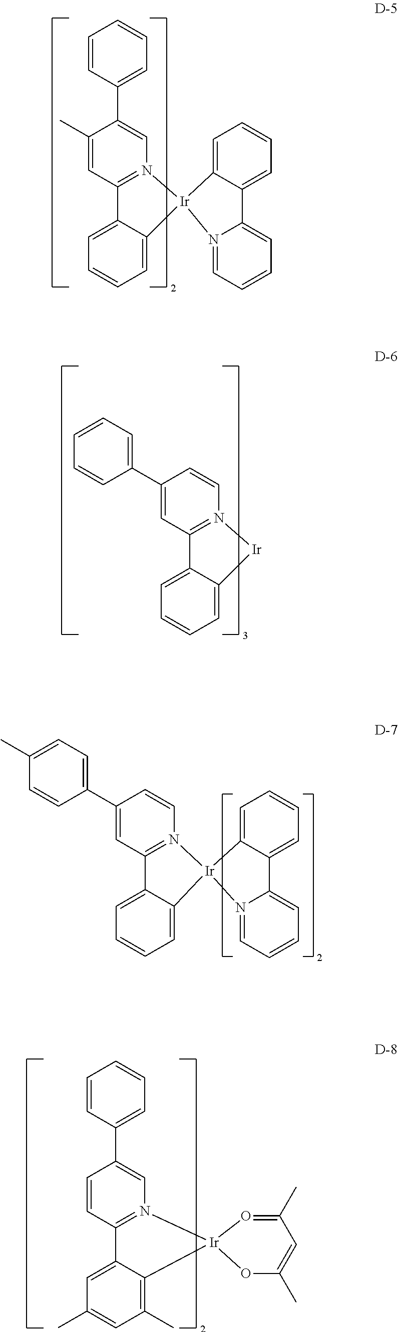

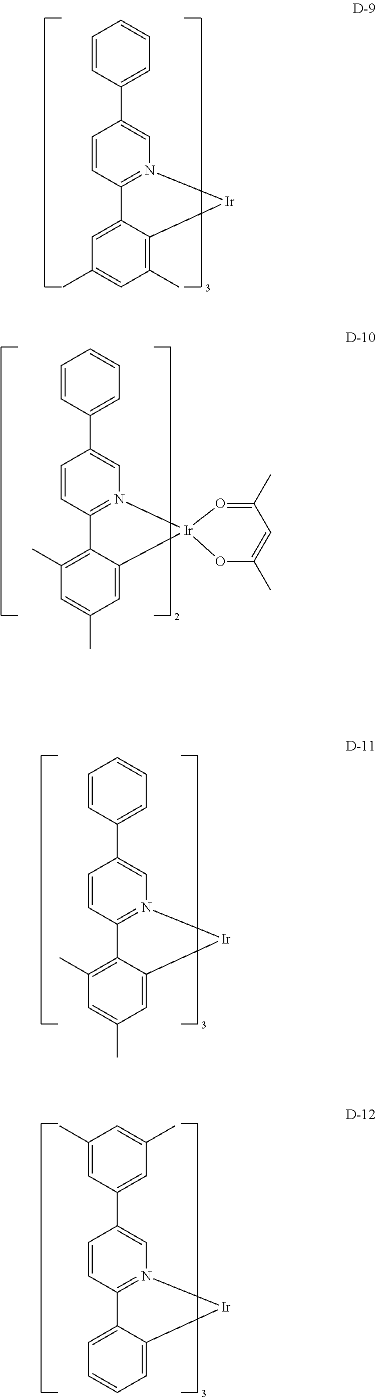

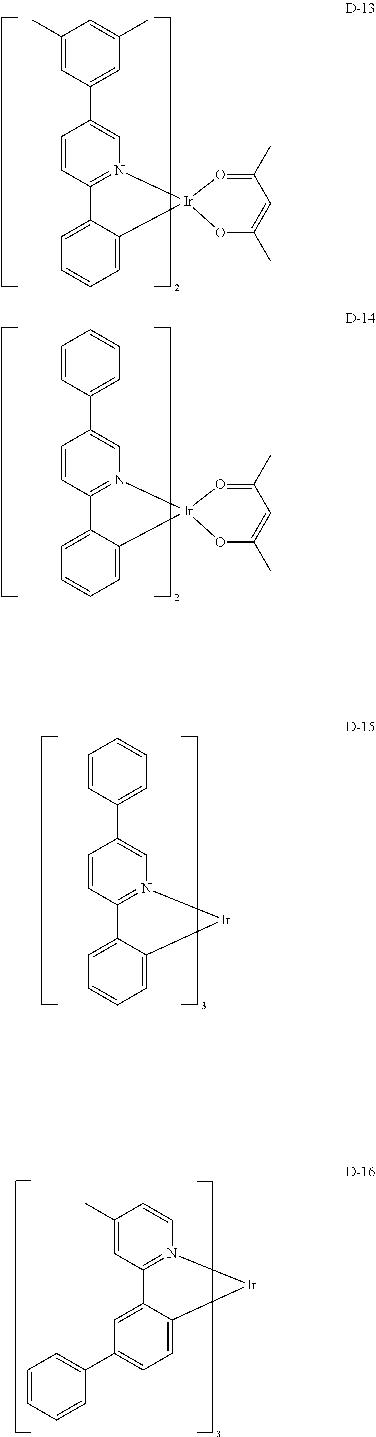

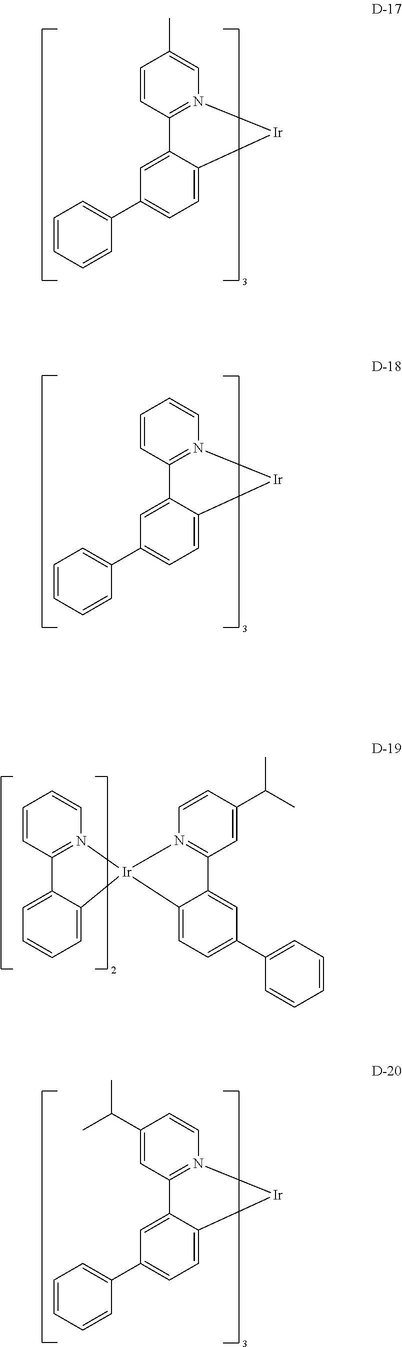

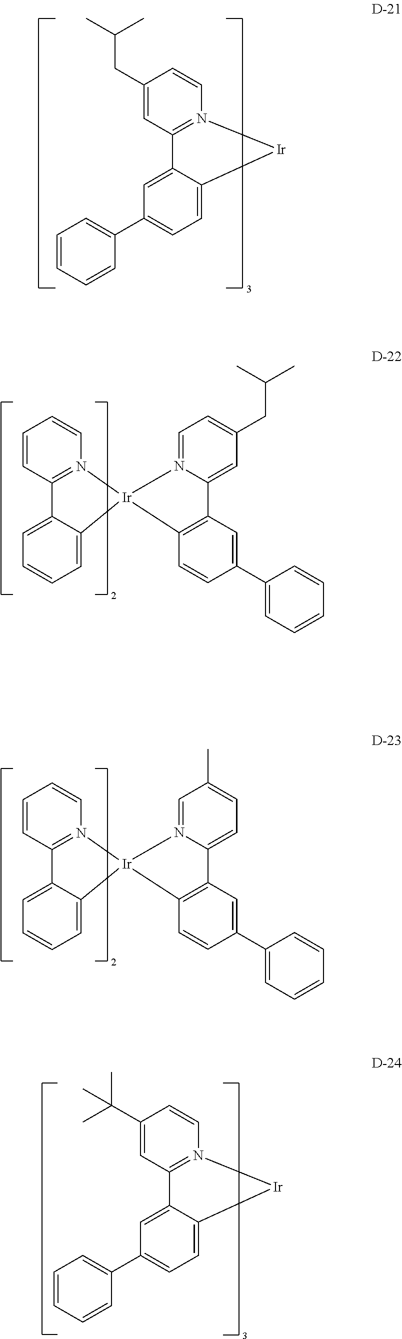

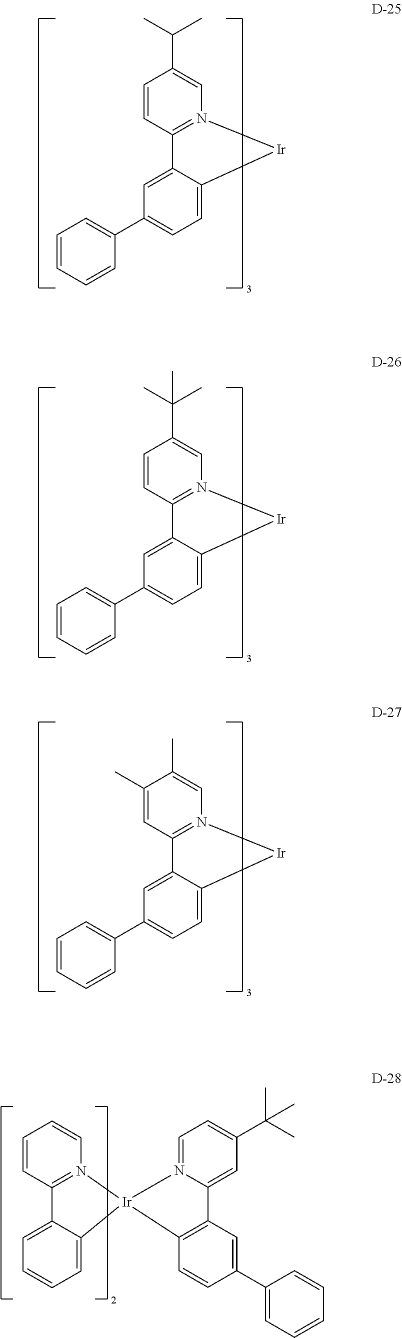

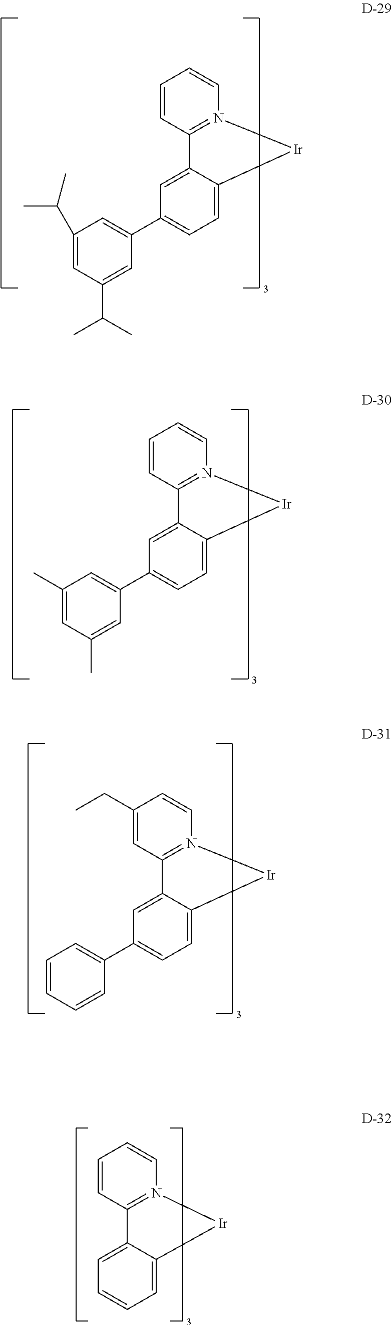

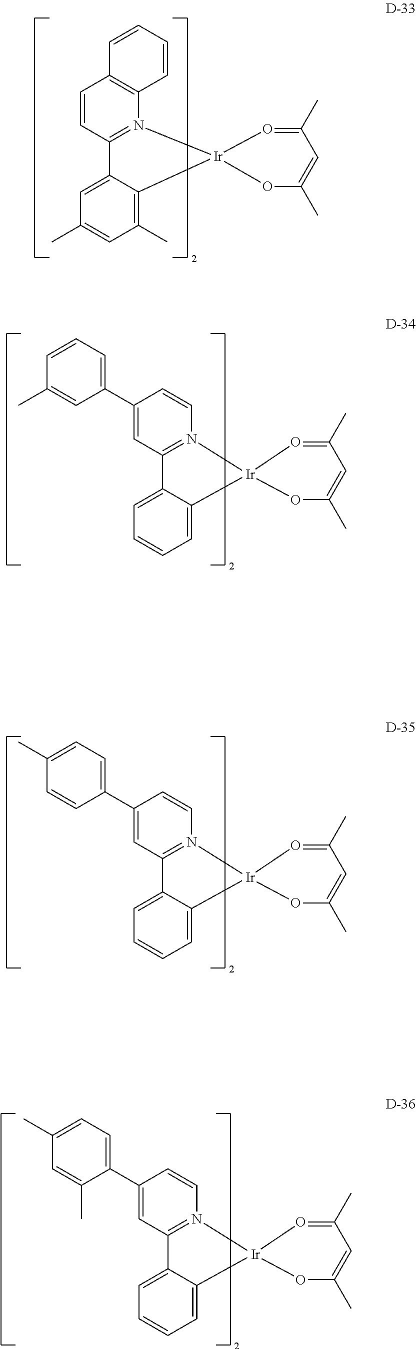

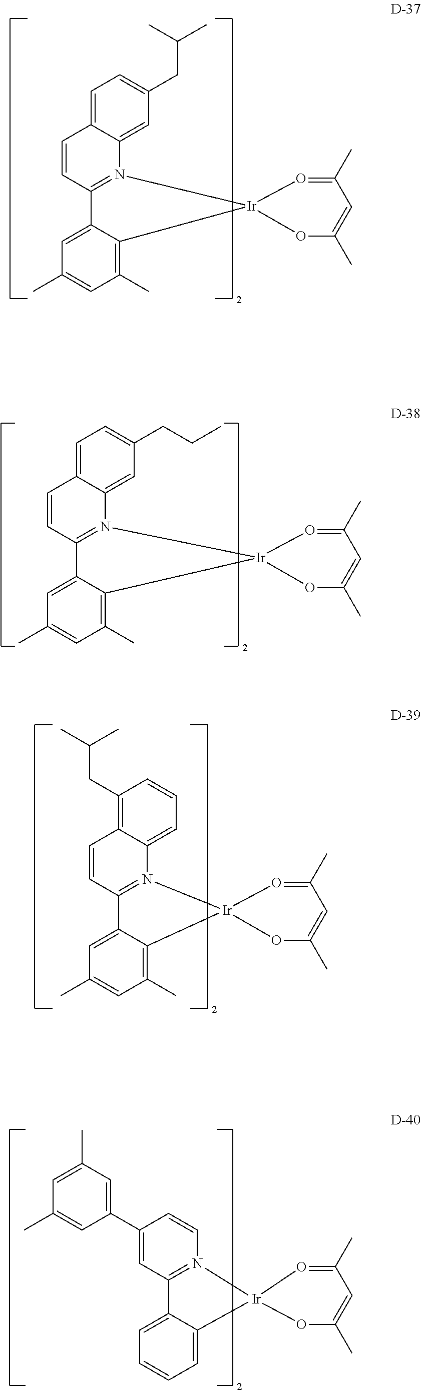

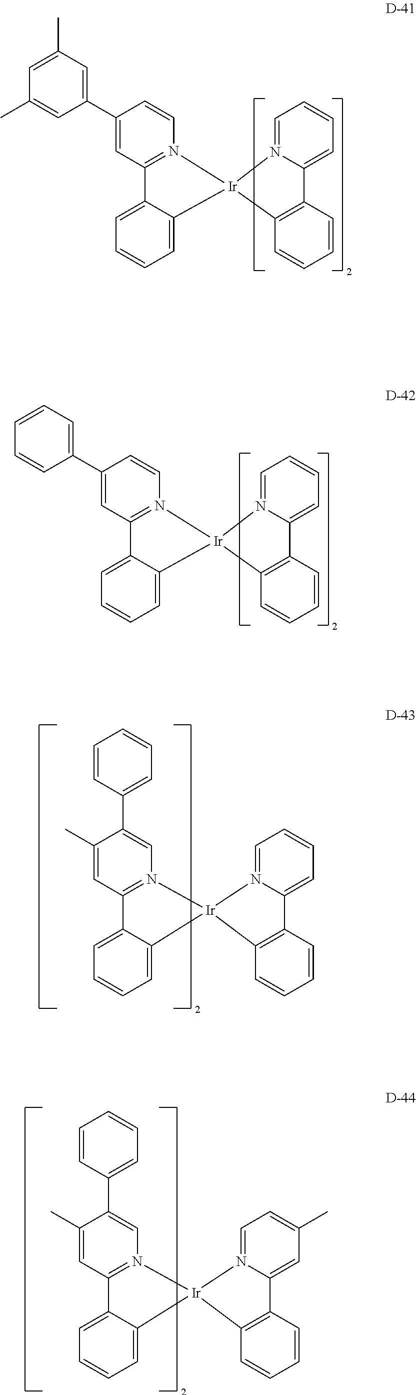

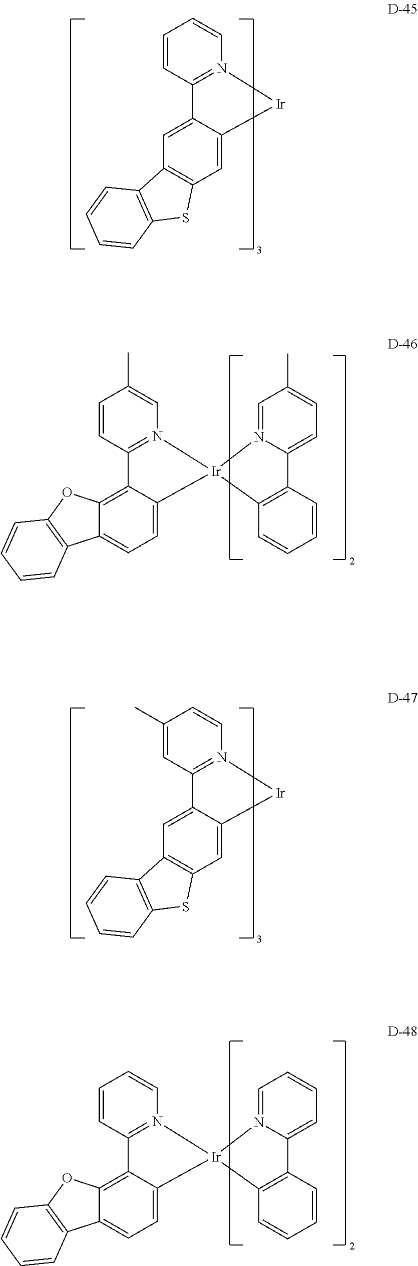

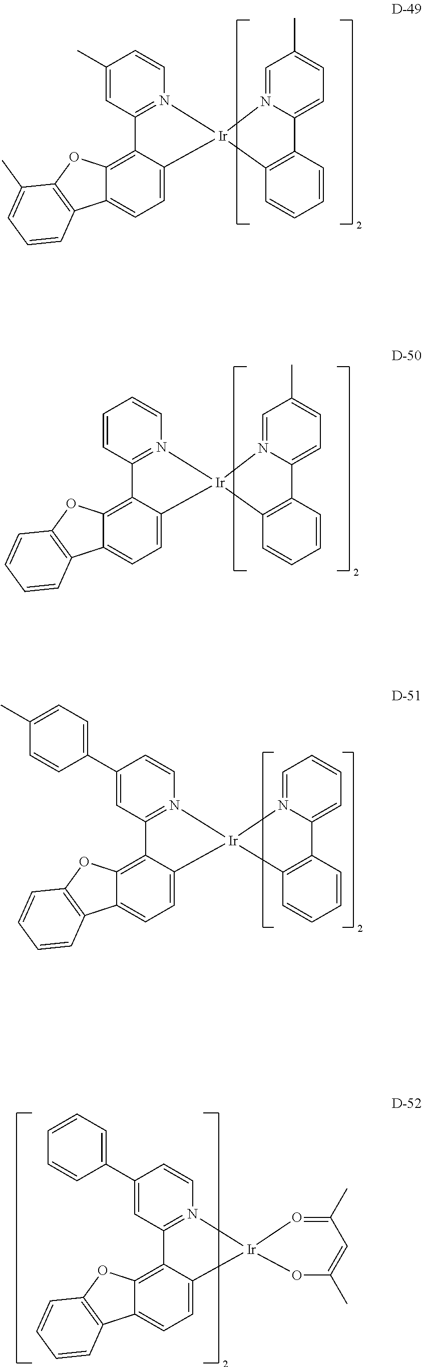

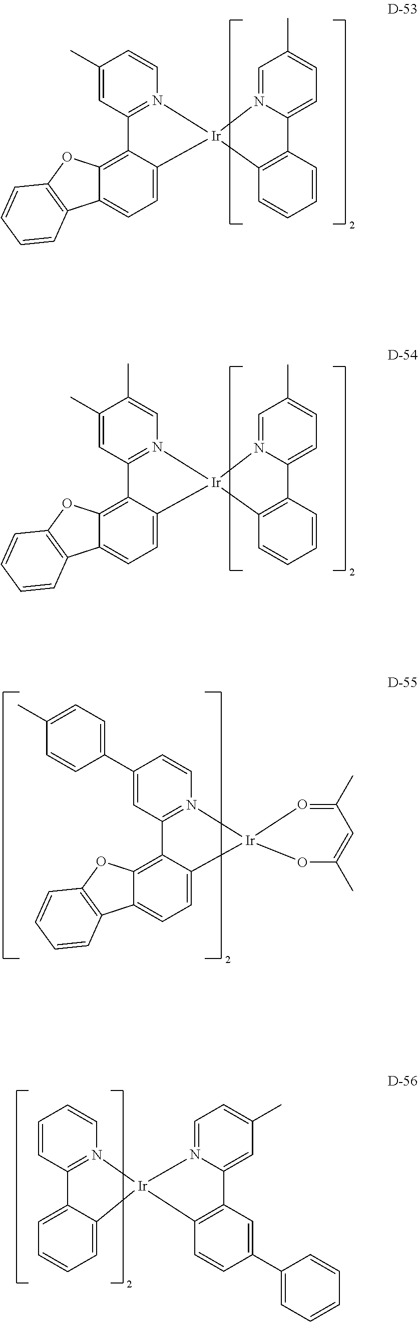

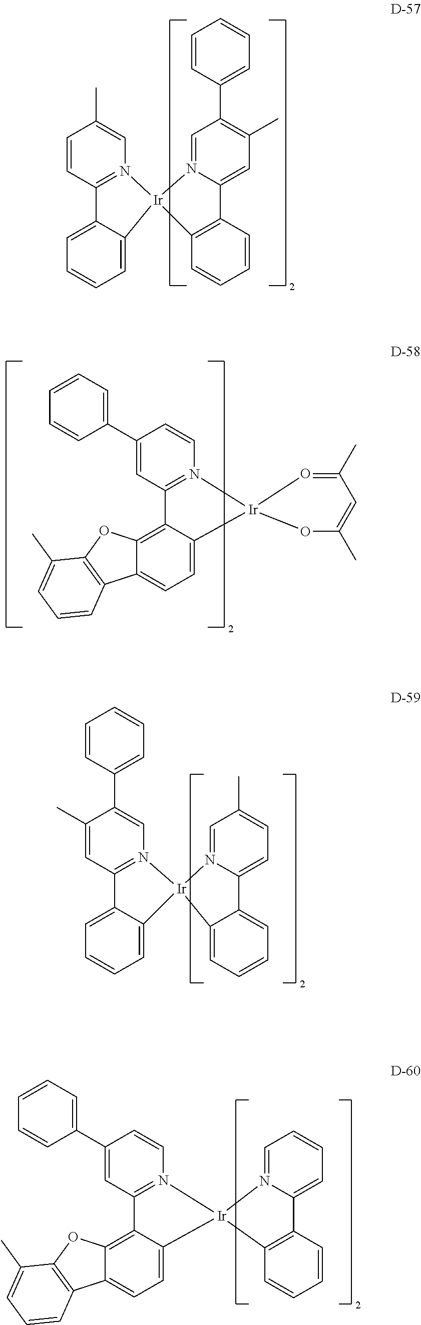

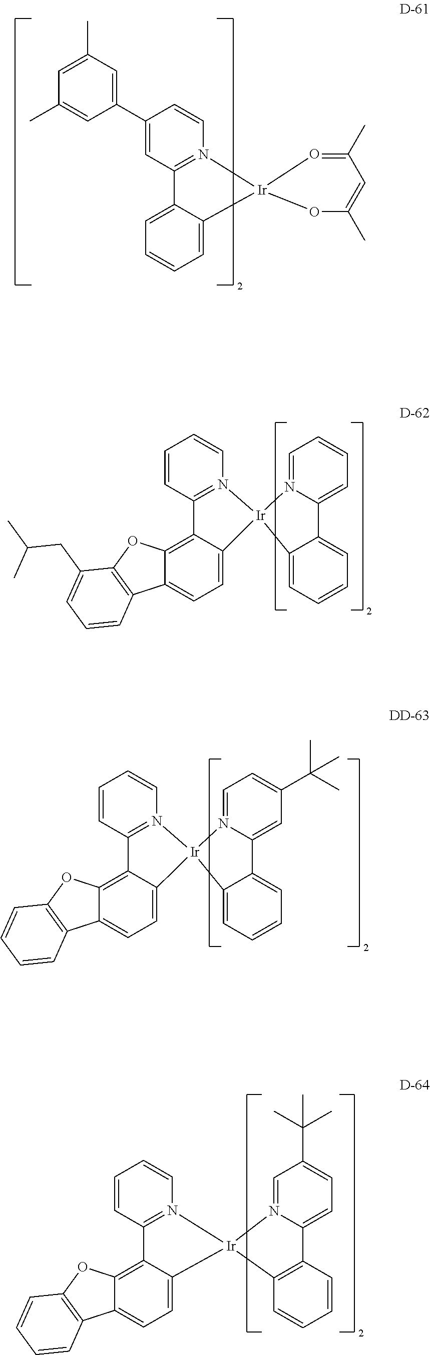

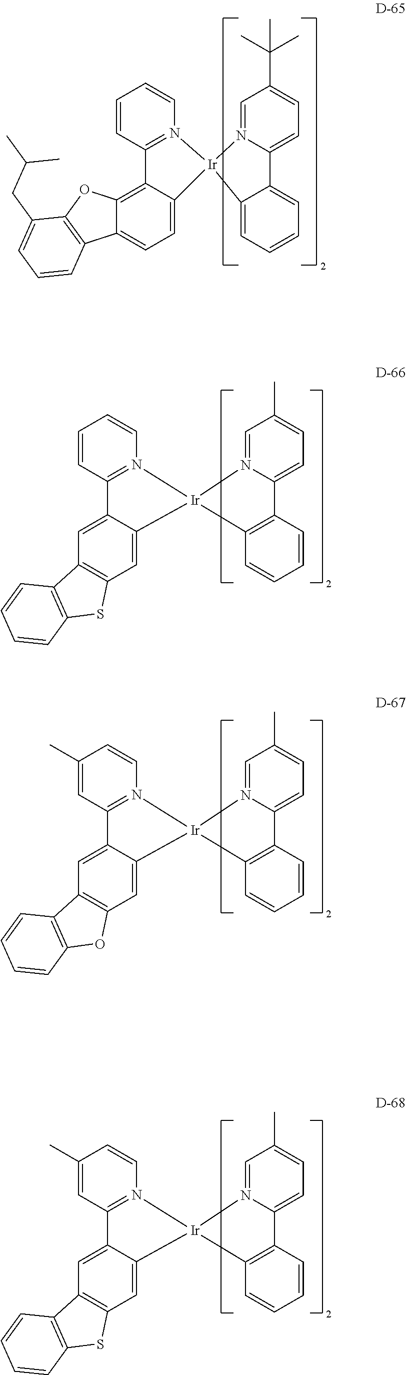

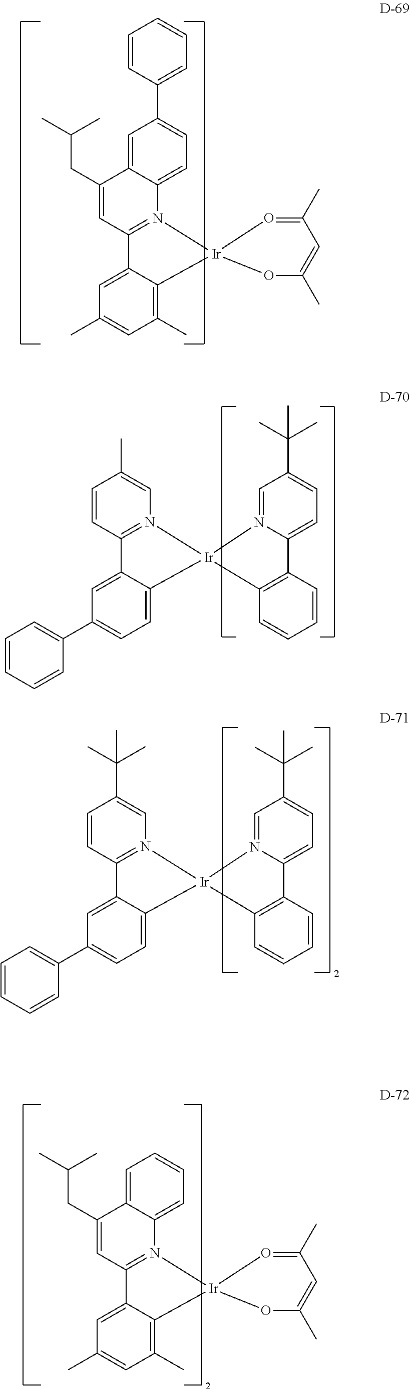

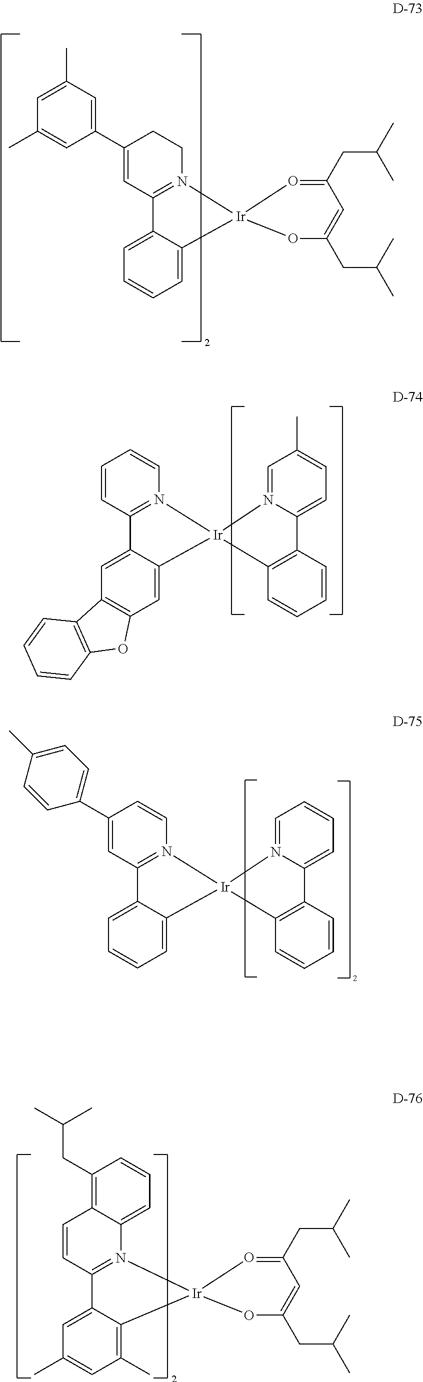

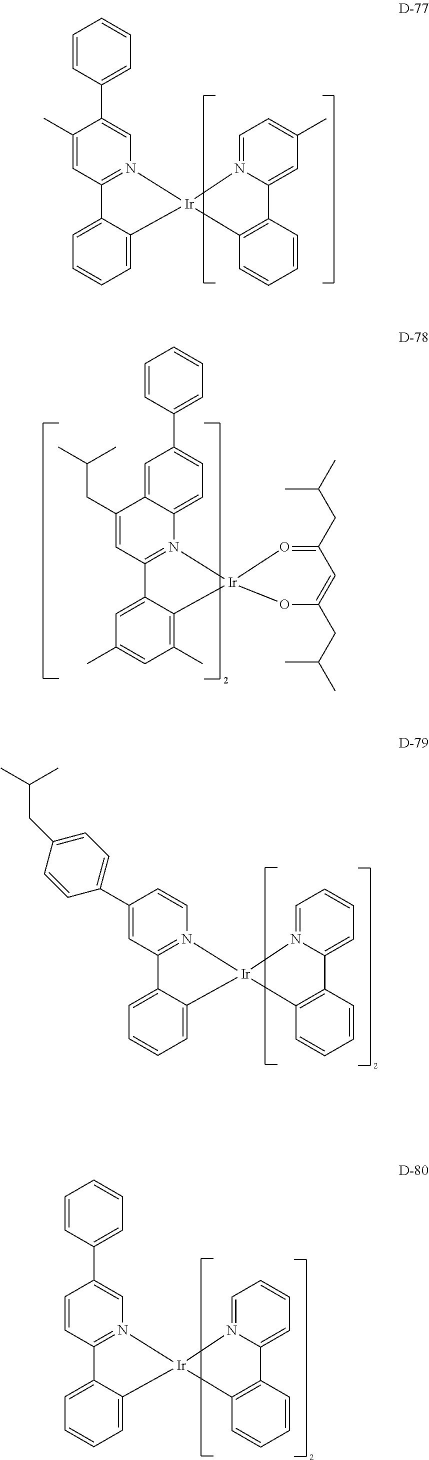

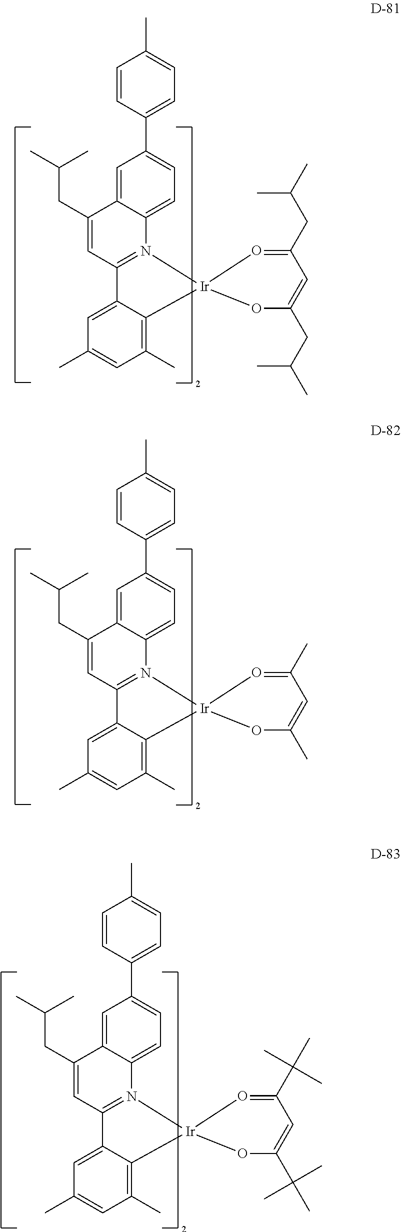

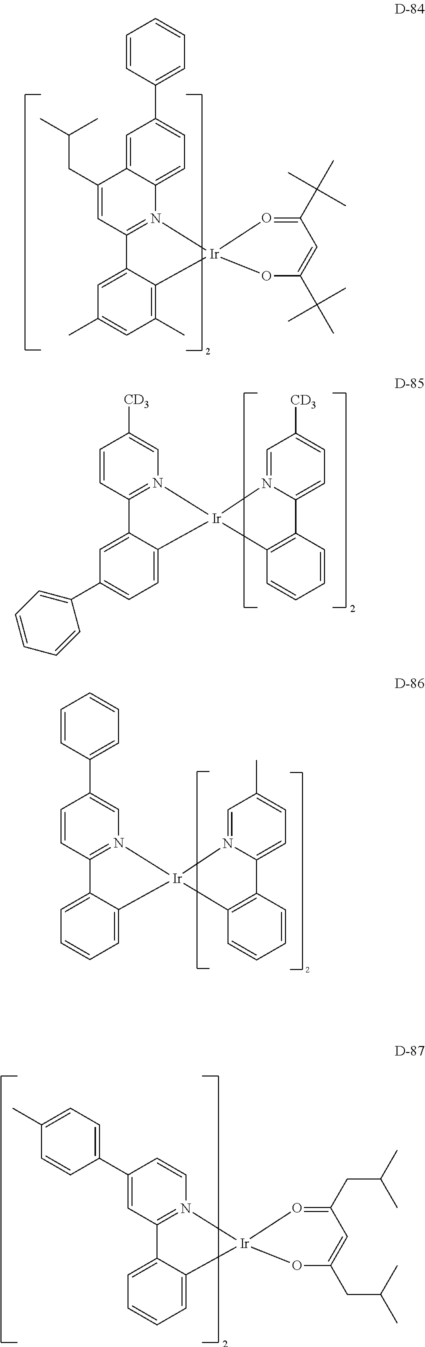

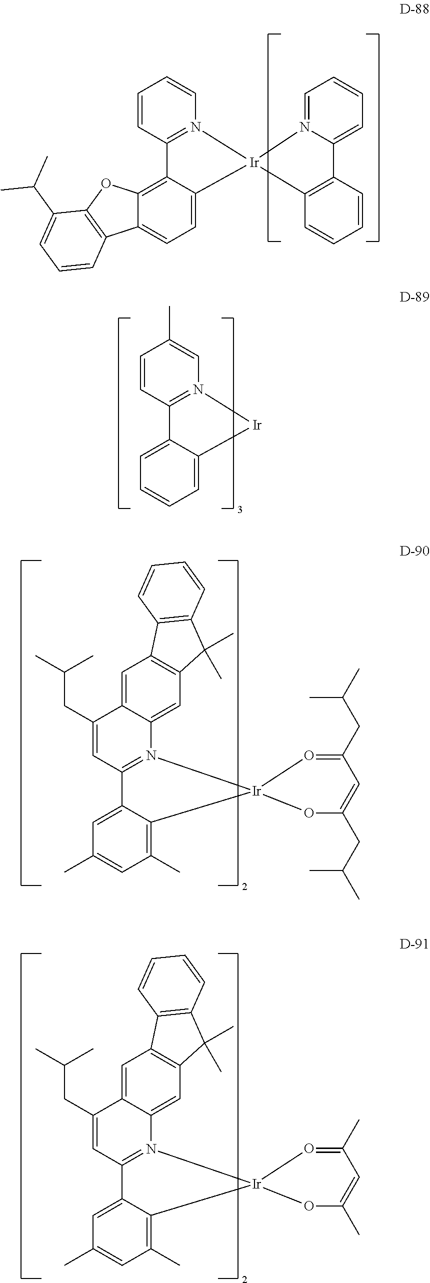

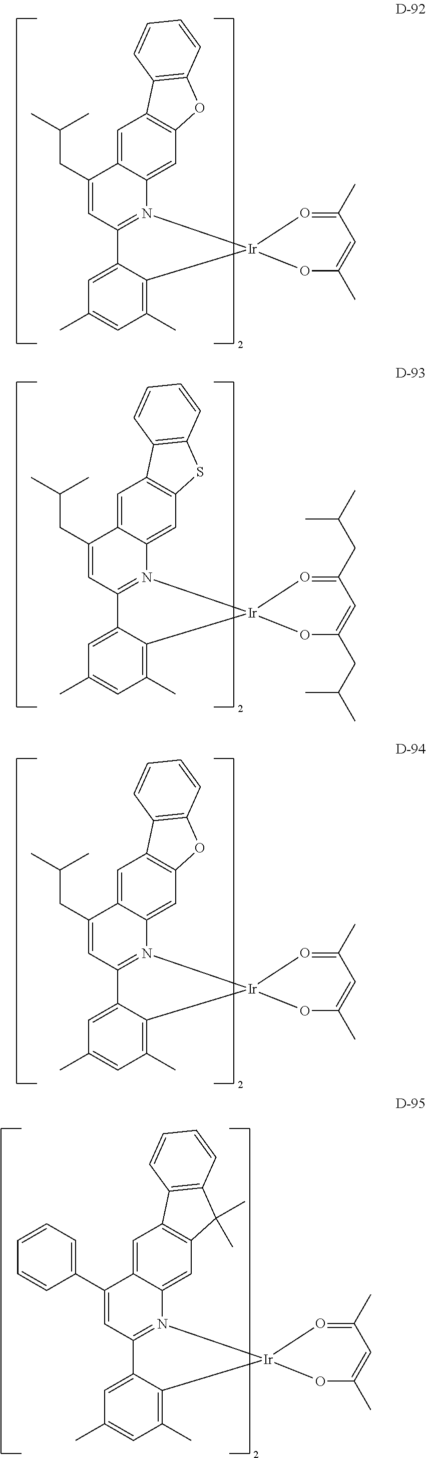

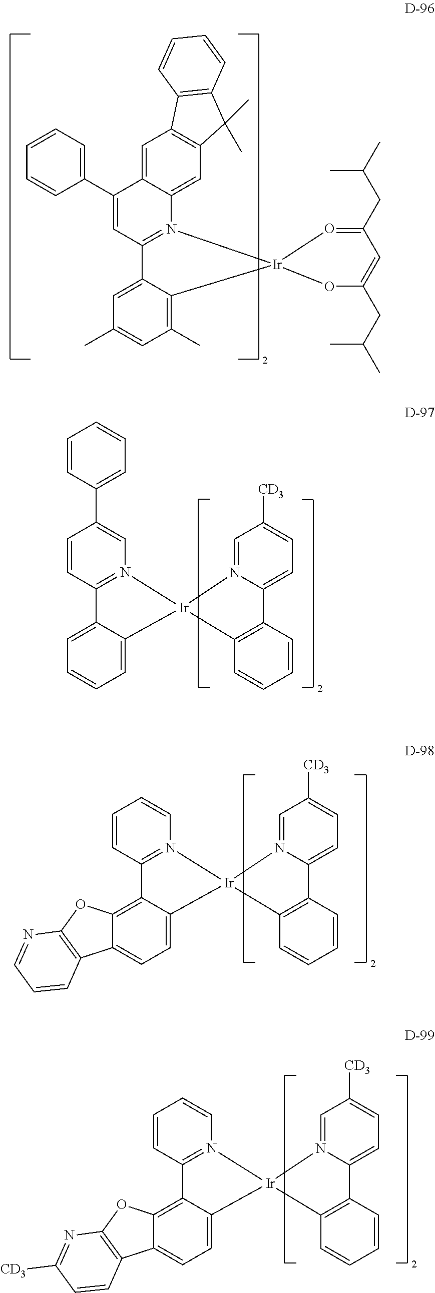

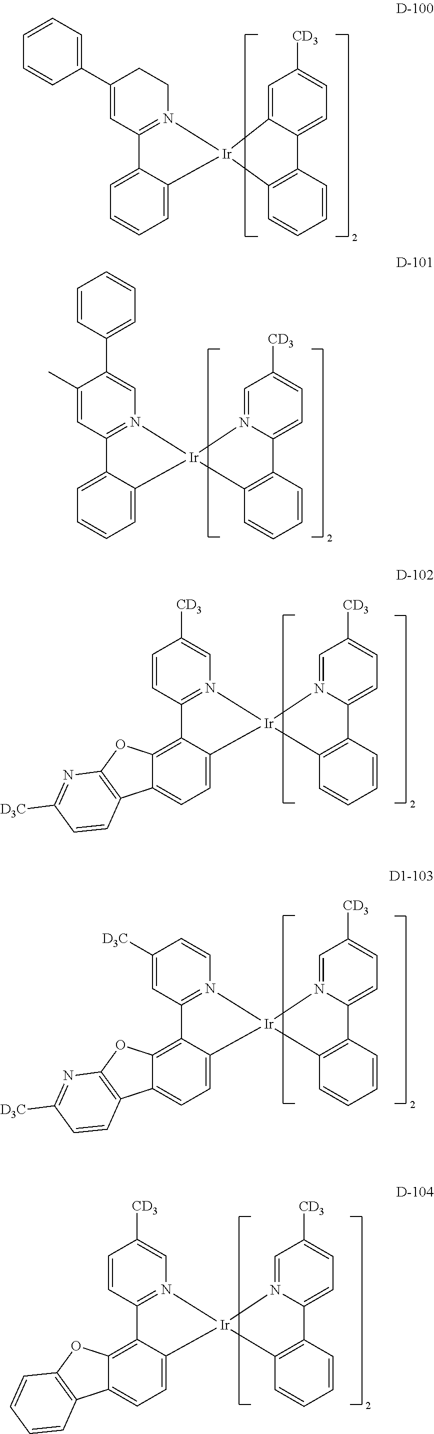

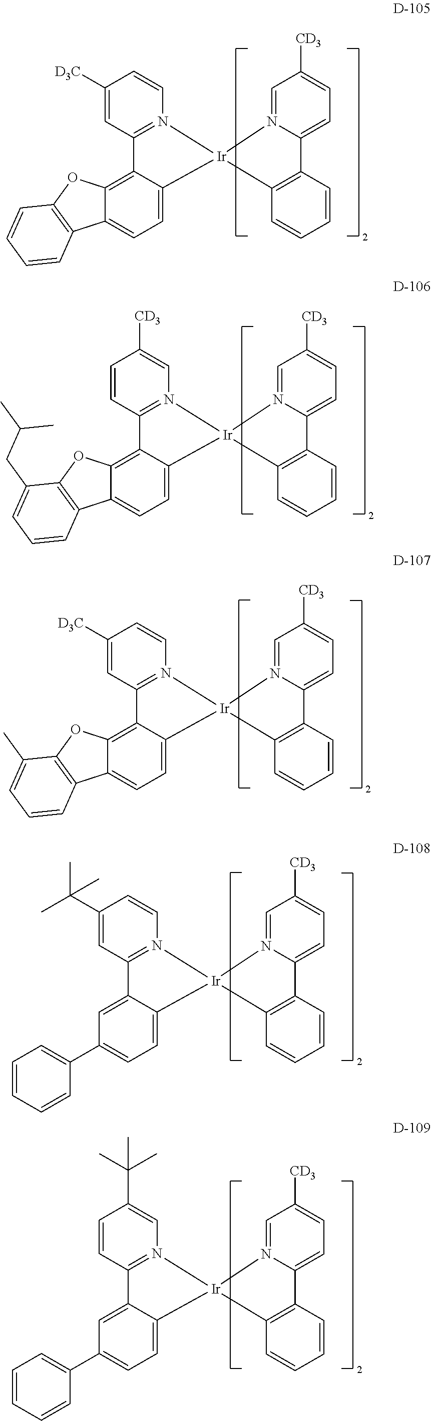

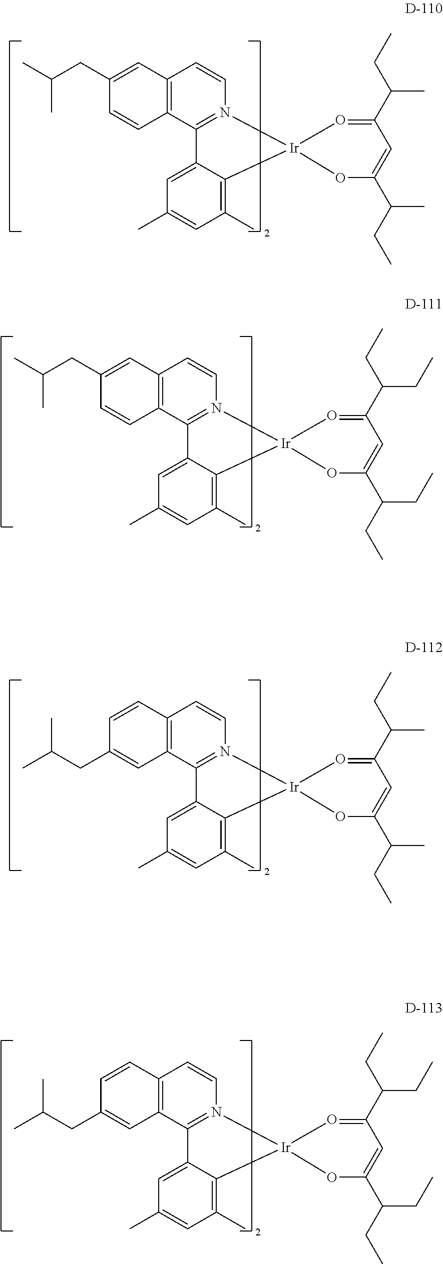

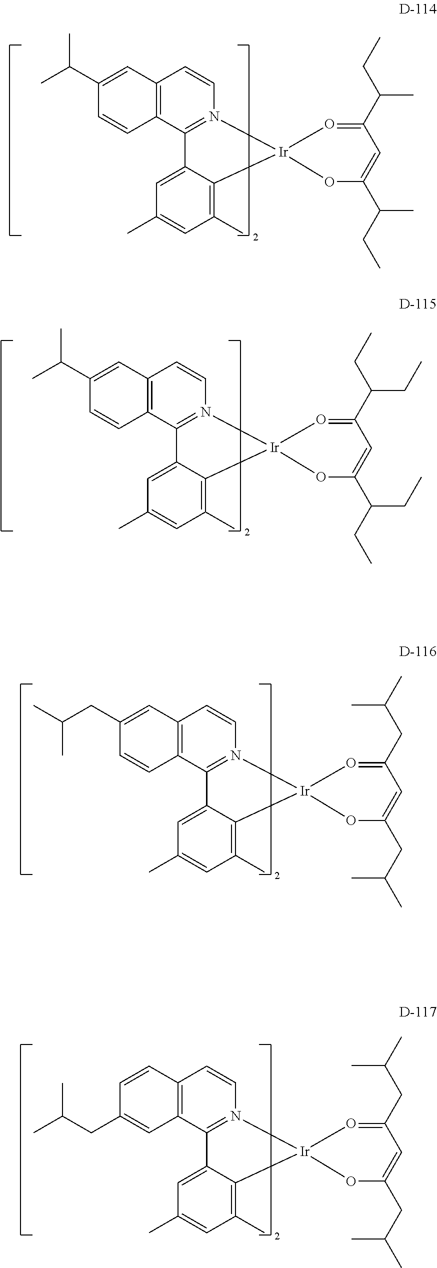

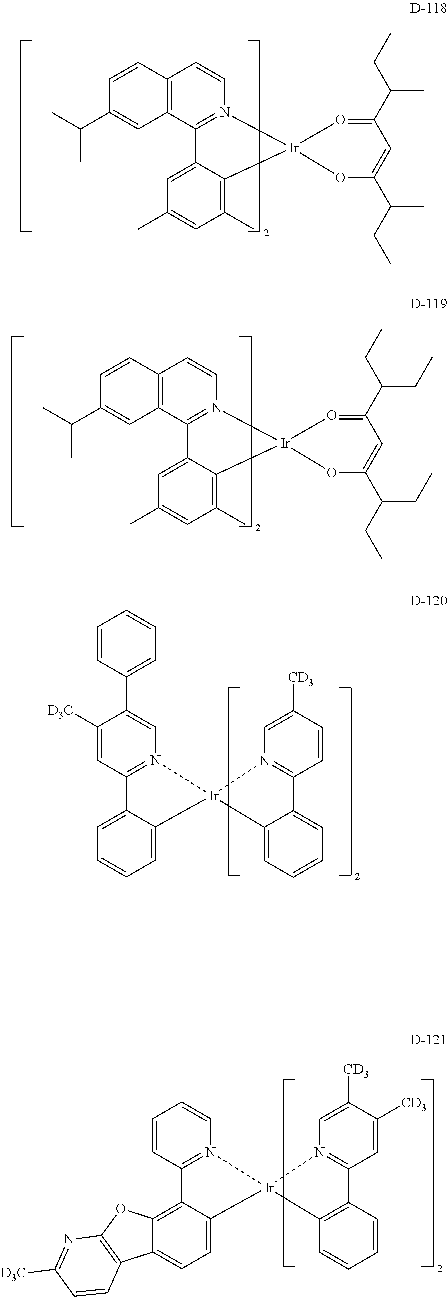

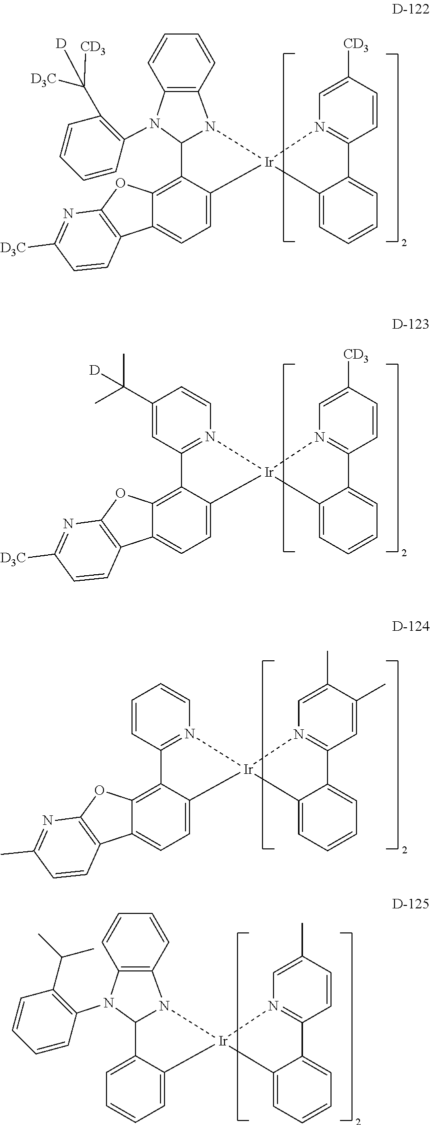

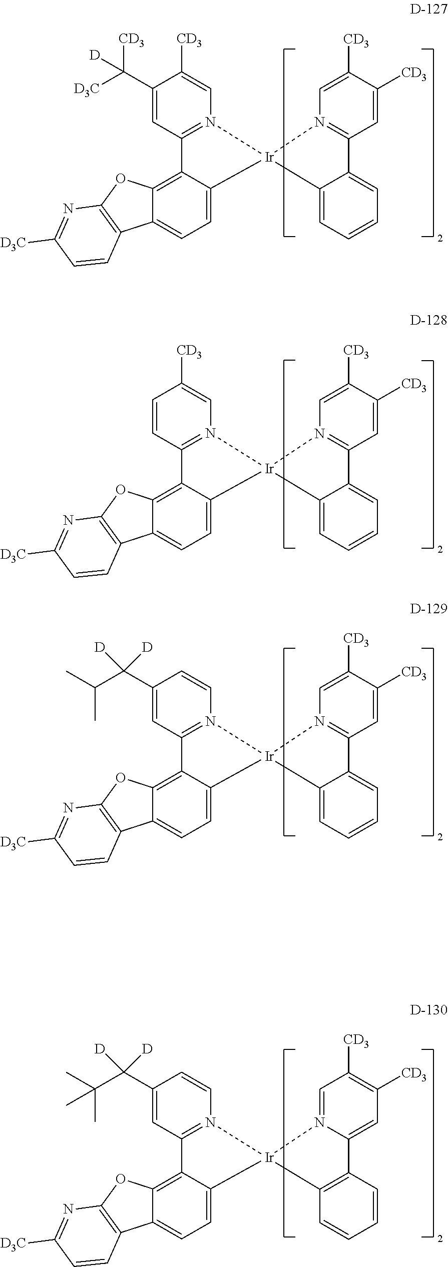

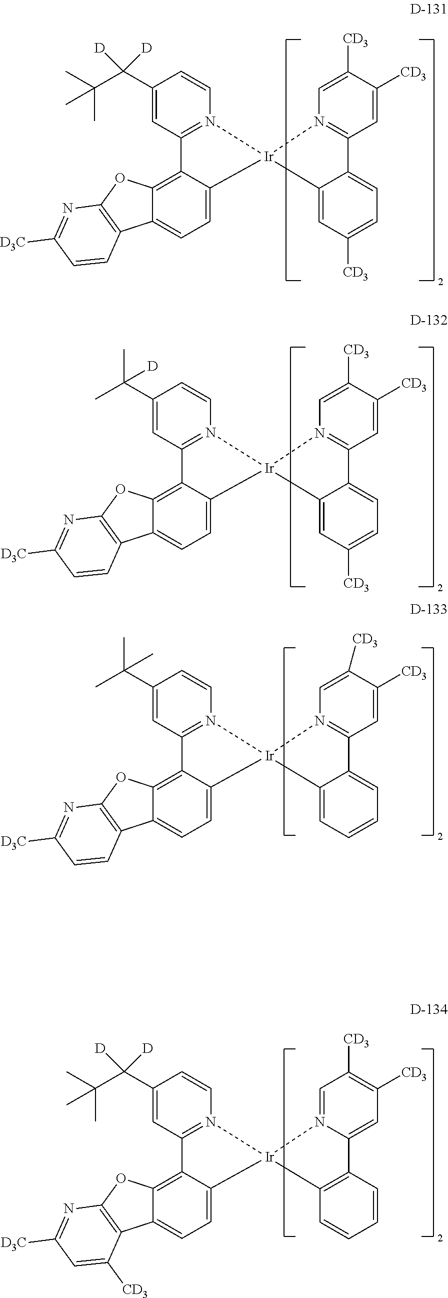

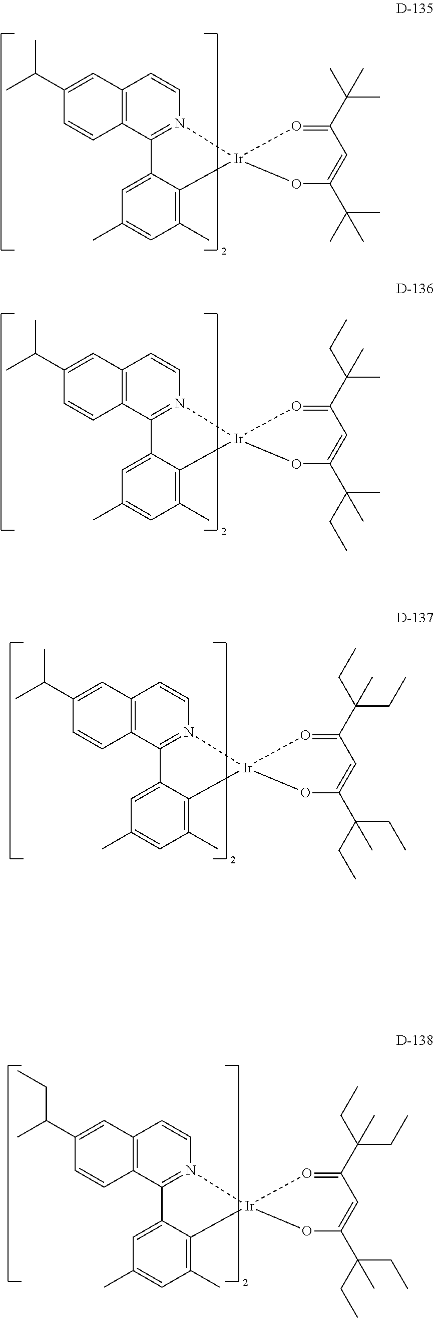

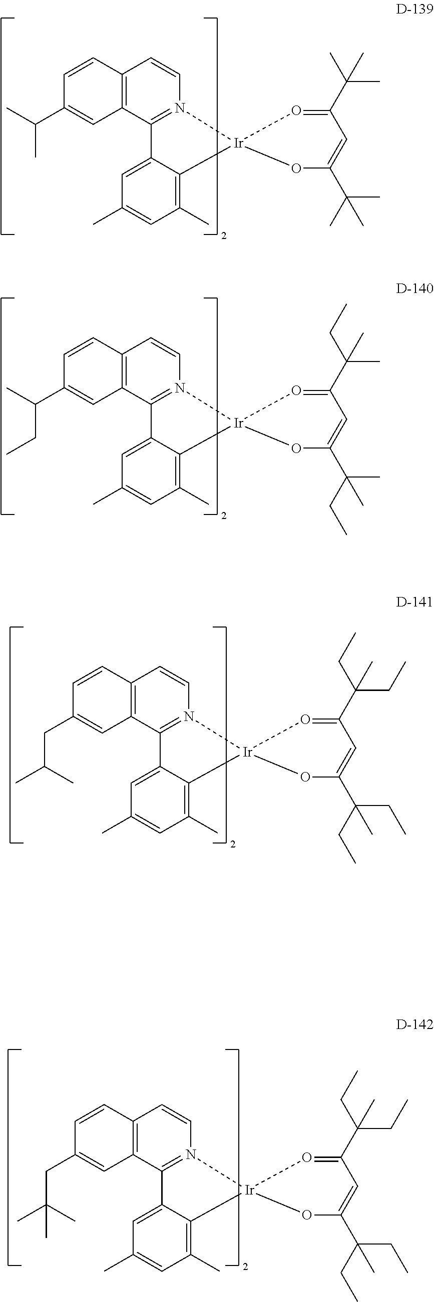

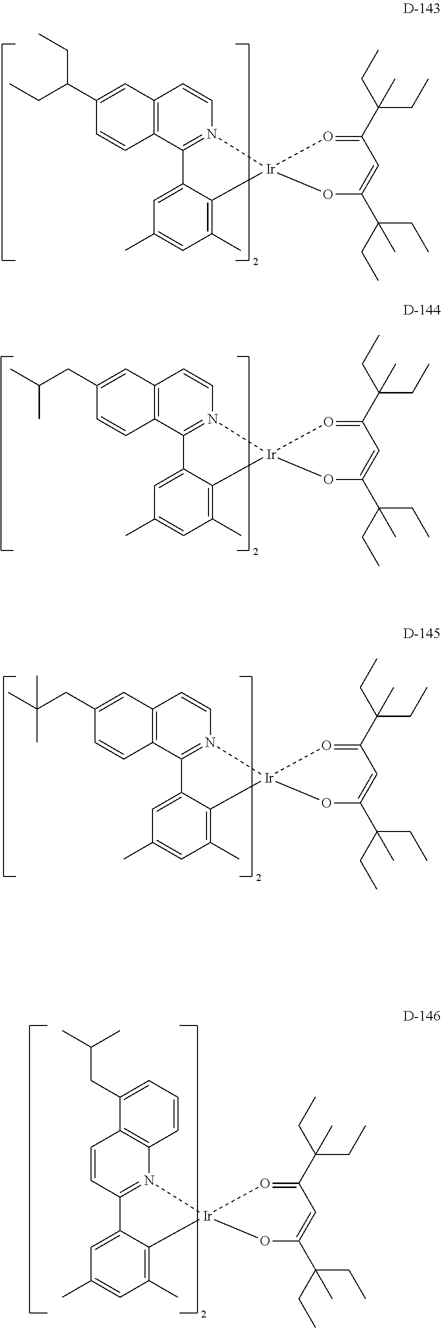

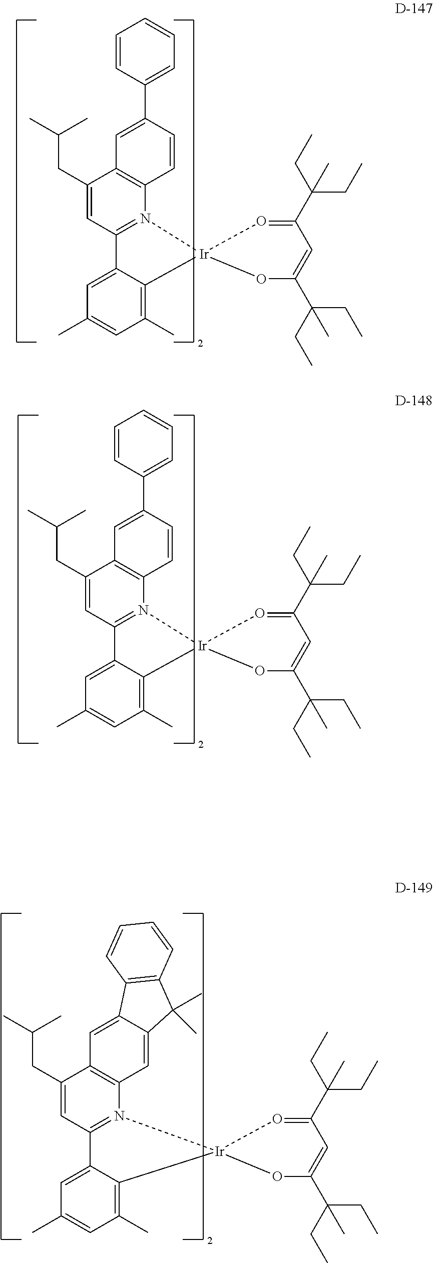

In an organic electroluminescent device of the present disclosure, a hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole transport layer or the electron blocking layer may also be multi-layers.

In addition, an electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The plurality of host materials according to the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

The first, second and third host compounds of the present disclosure may be film-formed by the above-listed methods, commonly by a co-evaporation process or a mixture-evaporation process. The co-evaporation is a mixed deposition method in which two or more materials are placed in a respective individual crucible source and a current is applied to both cells simultaneously to evaporate the materials. The mixture-evaporation is a mixed deposition method in which two or more materials are mixed in one crucible source before evaporating them, and a current is applied to the cell to evaporate the materials. Further, if the first, second and third host compounds are present in the same layer or different layers in an organic electroluminescent device, the three host compounds may individually form films. For example, the second host compound may be deposited after depositing the first and third host compounds.

The present disclosure may provide a display system using at least three types of host materials comprising the compound represented by formula 1, the compound represented by any one of formulas 2 to 4, and the compound represented by any one of formulas 1 to 4. In other words, it is possible to produce a display system or lighting system by using the plurality of host materials of the present disclosure. To be specific, it is possible to produce a display system, e.g., a display system for smart phones, tablets, notebooks, PCs, TVs, or cars; or a lighting system, e.g., an outdoor or indoor lighting system, by using the plurality of host materials of the present disclosure.

Originally, LUMO (lowest unoccupied molecular orbital) energy and HOMO (highest occupied molecular orbital) energy levels have negative values. However, for convenience, LUMO energy level and HOMO energy level are expressed in absolute values in the present disclosure. In addition, the LUMO energy levels are compared based on absolute values.

The values calculated by density functional theory (DFT) are used for LUMO and HOMO energy levels in the present disclosure. HOMO and LUMO energy levels of the light-emitting layer, which will be described later, are defined as a first host HOMO (Aₕ₁), a second host HOMO (Aₕ₂) and a third host HOMO (Aₕ₃), and a first host LUMO (Aₗ₁), a second host LUMO (Aₗ₂) and a third host LUMO (Aₗ₃). The results are intended to explain the tendency of alternative devices according to the overall HOMO and LUMO energy groups, and the results different from the above may be obtained depending on the intrinsic properties of specific derivatives and the stability of materials.

In an organic electroluminescent device of the present disclosure, assuming that the first and third host materials having strong hole transport properties are used with the second host having strong electron transport properties, the HOMO energy level of the first host compound may be greater than that of the third host compound. The difference of the HOMO energy levels between the first host compound and the third host compound may be specifically about 0.5 eV or less, preferably about 0.3 eV or less. For example, the HOMO energy levels of the first and third host compounds may be about 5.1 eV and about 4.8 eV, respectively, and thus, the difference between their HOMO energy levels may be about 0.3 eV.

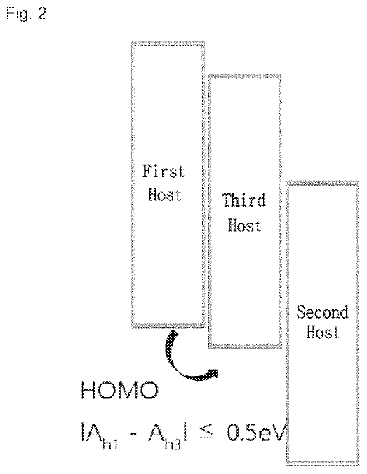

With reference to FIG. 2, when each of the first and third host materials has stronger hole transport properties than the second host material, the HOMO energy level of the light-emitting layer comprising the plurality of host materials may satisfy the following Equation 1, preferably, the following Equation 2.

$$\vert {A}_{h 1} - {A}_{h 3} \vert \leq 0.5 eV \qquad [ Equation 1 ]$$

$$\vert {A}_{h 1} - {A}_{h 3} \vert \leq 0.3 eV \qquad [ Equation 2 ]$$

In Equations 1 and 2, Aₕ₁ represents the HOMO energy level of the first host material, and Aₕ₃ represents the HOMO energy level of the third host material.

The HOMO barrier between the first host compound and the hole transport layer may be a factor in increasing the driving voltage, and there is almost no HOMO barrier between the third host compound and the hole transport layer, so the hole trap is not smooth, and thus there is a significant limitation in increasing the efficiency. However, when the third host compound comprises a compound having a low HOMO energy level among the compounds of formula 1 and a low barrier to the hole transport layer, transporting holes to the host compound becomes easier compared to each compound, and at the same time, efficiency and lifetime can be improved.

In addition, assuming that the first host material having strong hole transport properties are used with the second and third hosts having strong electron transport properties, the LUMO energy level of the second host compound may be greater than that of the third host compound. The difference between the LUMO energy levels of the second and third host compounds may be specifically about 0.5 eV or less, preferably about 0.3 eV or less. For example, the LUMO energy levels of the second and third host compounds may be about 1.8 eV and about 2.1 eV, respectively, and thus, the difference between their LUMO energy levels may be about 0.3 eV.

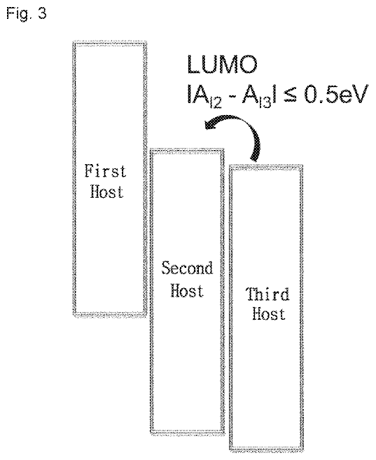

With reference to FIG. 3, when each of the second and third host materials has stronger electron transport properties than the first host material, the LUMO energy level of the light-emitting layer comprising the plurality of host materials may satisfy the following Equation 3, preferably, the following Equation 4.

$$\vert {A}_{12} - {A}_{13} \vert \leq 0.5 eV \qquad [ Equation 3 ]$$

$$\vert {A}_{12} - {A}_{13} \vert \leq 0.3 eV \qquad [ Equation 4 ]$$

In Equations 3 and 4, Aₗ₂ represents the LUMO energy level of the second host material, and Aₗ₃ represents the LUMO energy level of the third host material.

Since the second host compound has a high LUMO energy, transporting electrons is not easy, so there is a limitation in increasing efficiency properties. The third host compound has a significant limitation in increasing lifetime properties due to relatively fast electron transport. However, when the second and third host compounds are mixed, the efficiency and lifetime can be increased at the same time by maintaining the appropriate current properties and controlling the ratio of the hole and electrons in the light-emitting layer. Therefore, the organic electroluminescent device of the present disclosure may have a low driving voltage, excellent luminous efficiency, and a long lifetime.

Hereinafter, the preparation method of the compounds according to the present disclosure, the properties thereof, and the properties of the OLED comprising the plurality of host materials according to the present disclosure will be explained in detail with reference to the representative compounds of the present disclosure. The following examples only describe the properties of the OLED comprising the compound according to the present disclosure, but the present disclosure is not limited to the following examples.

### Example 1: Preparation of Compound H1-222

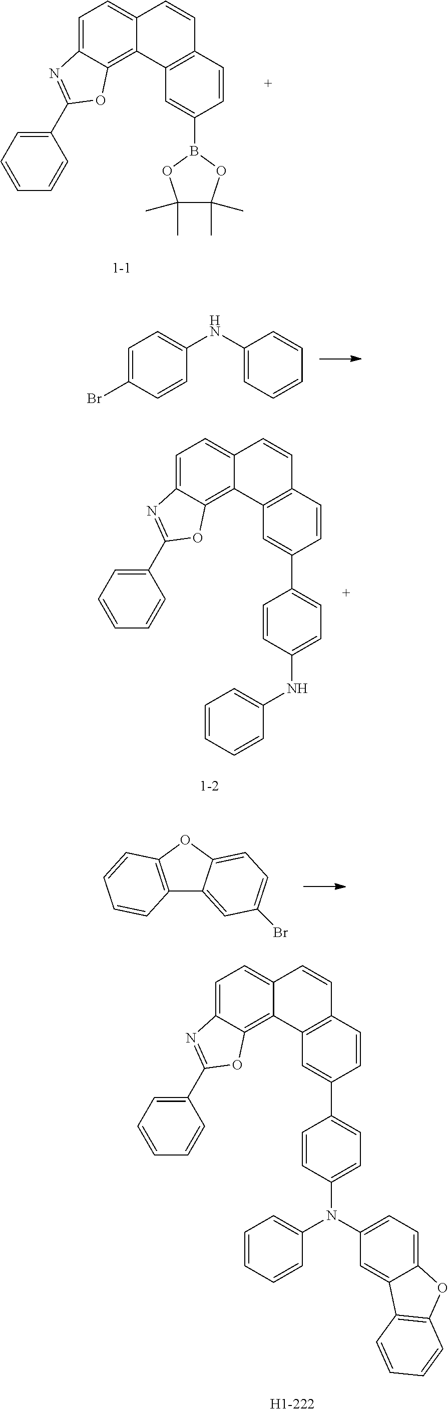

### Synthesis of Compound 1-2

Compound 1-1 (50 g, 118.6 mmol), 4-bromo-N-phenylaniline (29.5 g, 118.9 mmol), Pd(OAc)₂ (0.2664 g, 1.18 mmol), S-Phos (0.9744 g, 2.3 mmol), and K₂CO₃ (41 g, 296.6 mmol) were dissolved in 600 mL of toluene and 95.7 mL of H₂O, and the mixture was stirred under reflux for 2 hours. The mixture was cooled to room temperature, and filtered through Celite. The obtained solid was separated by column chromatography to obtain compound 1-2 (45 g, yield: 82%).

### Synthesis of Compound H1-222

Compound 1-2 (45 g, 97.2 mmol), 2-bromobenzofuran (24 g, 97.1 mmol), Pd(OAc)₂ (0.2183 g, 0.97 mmol), S-Phos (0.7887 g, 1.92 mmol), and NaOt-Bu (23.4 g, 243.4 mmol) were dissolved in 450 mL of o-xylene, and the mixture was stirred under reflux for 3 hours. The mixture was cooled to room temperature, and filtered through Celite. The obtained solid was separated by column chromatography to obtain compound H1-222 (18 g, yield: 29%).

| | MW | M.P. |
|---|---|---|
| H1-222 | 628.73 | 252° C. |

### Example 2: Preparation of Compound H1-221

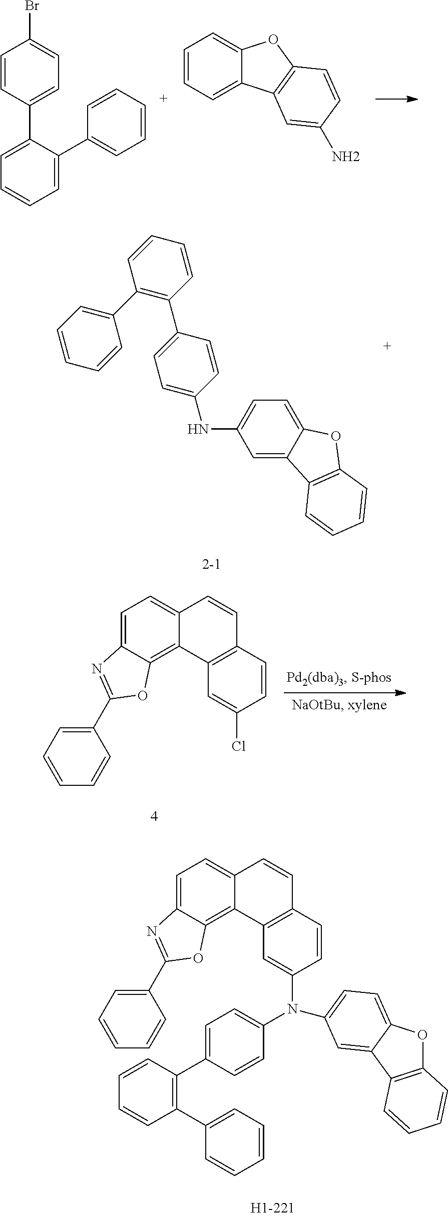

### Synthesis of Compound 2-1

4-bromo-1,1′:2′,1″-terphenyl (10.0 g, 32.34 mmol), 8-aminodibenzo[b,d]furan-2-yl (8.8 g, 48.51 mmol), PdCl₂(Amphos)₂ (2.3 g, 3.23 mmol), and NaOt-Bu (4.6 g, 48.51 mmol) were dissolved in 161 mL of o-xylene, and the mixture was stirred under reflux for 2 hours. The mixture was cooled to room temperature, and filtered through Celite. The obtained solid was separated by column chromatography to obtain compound 2-1 (8.6 g, yield: 64.6%).

### Synthesis of Compound H1-221

Compound 2-1 (8.6 g, 20.90 mmol), compound 4 (8.3 g, 25.08 mmol), Pd₂(dba)₃ (1.0 g, 1.05 mmol), S-Phos (900 mg, 2.09 mmol), and NaOt-Bu (5.0 g, 52.25 mmol) were dissolved in 140 mL of o-xylene, and the mixture was stirred under reflux for 3 hours. The mixture was cooled to room temperature, and filtered through Celite. The obtained solid was separated by column chromatography to obtain compound H1-221 (5.8 g, yield: 39.4%).

| | MW | M.P. |
|---|---|---|
| H1-221 | 704.83 | 168° C. |

### Example 3: Preparation of Compound H2-745

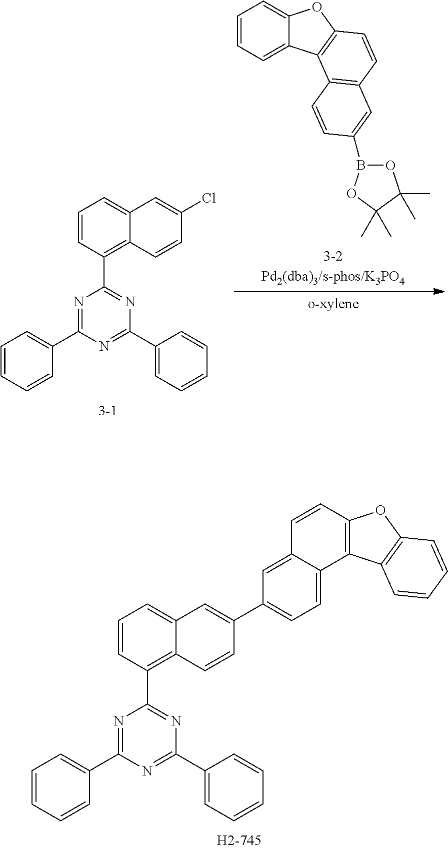 [SMILES: Clc1ccc2c(-c3nc(-c4ccccc4)nc(-c4ccccc4)n3)cccc2c1 ; CC1(C)OB(c2ccc3c(ccc4oc5ccccc5c43)c2)OC1(C)C ; c1ccc(-c2nc(-c3ccccc3)nc(-c3cccc4cc(-c5ccc6c(ccc7oc8ccccc8c76)c5)ccc34)n2)cc1]

In a flask, compound 3-1 (5 g, 12.69 mmol), compound 3-2 (8.7 g, 25.38 mmol), Pd₂(dba)₃ (0.58 g, 0.634 mmol), s-phos (0.52 g, 1.269 mmol), and K₃PO₄ (6.7 g, 31.73 mmol) were dissolved in 65 mL of o-xylene, and the mixture was stirred under reflux for 15 hours. After completion of the reaction, the mixture was cooled to room temperature, and methanol was added, and then the solid was filtered. Thereafter, the resulting solid was dissolved in chlorobenzene, and separated by silica filter to obtain compound H2-745 (3.3 g, yield: 45%).

| | MW | M.P. |
|---|---|---|
| H2-745 | 575.6 | 250.9° C. |

### Device Examples 1 to 15: Producing a Red OLED Deposited with a Plurality of Host Materials According to the Present Disclosure as Hosts

OLEDs according to the present disclosure were produced. A transparent electrode indium tin oxide (ITO) thin film (10 O/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm on the ITO substrate. Next, compound HT-1 was deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: The first host compound, the second host compound, and the third host compound shown in Table 1 below were introduced into three cells of the vacuum vapor deposition apparatus as hosts, and compound D-39 was introduced into another cell as a dopant. The three host materials were evaporated at a rate of 0.25:0.5:0.25 (the first host: the second host: the third host) and the dopant material was simultaneously evaporated at a different rate, and the dopant was deposited in a doping amount of 3 wt % based on the total amount of the hosts and the dopant to form a light-emitting layer having a thickness of 40 nm on the second hole transport layer. Thereafter, compound ETL-1 and compound EIL-1 as electron transport materials were evaporated in a weight ratio of 50:50 to deposit an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Examples 1 to 27: Producing an OLED Comprising Comparative Compounds as Hosts

OLEDs were produced in the same manner as in Device Example 1, except that the first host compound, the second host compound, or the third host compound shown in Table 1 below was used as the single host of the light-emitting layer.

The driving voltage, luminous efficiency, and light-emitting color at a luminance of 1,000 nit, and the time taken for luminance to decrease from 100% to 95% (lifetime; T95) at a luminance of 10,000 nit of the OLEDs produced in Device Examples 1 to 15, and Comparative Examples 1 to 27 are provided in Table 1 below.

TABLE 1

| | | | | Driving | Luminous | Light- | Lifetime |
|---|---|---|---|---|---|---|---|
| | First | Second | Third | Voltage | Efficiency | Emitting | (T95) |
| | Host | Host | Host | M | [cd/A] | Color | [hr] |
| Device | H1-222 | H2-745 | H1-221 | 3.1 | 36.4 | Red | 121 |
| Example 1 | | | | | | | |
| Device | H1-226 | H2-745 | H1-221 | 3.1 | 35.8 | Red | 184 |
| Example 2 | | | | | | | |
| Device | H1-227 | H2-746 | H1-228 | 3.0 | 34.5 | Red | 318 |
| Example 3 | | | | | | | |
| Device | H1-227 | H2-746 | H1-229 | 3.1 | 34.4 | Red | 210 |
| Example 4 | | | | | | | |
| Device | H1-227 | H2-746 | H1-226 | 3.0 | 34.1 | Red | 246 |
| Example 5 | | | | | | | |
| Device | H1-230 | H2-712 | H1-225 | 2.9 | 36.8 | Red | 260 |
| Example 6 | | | | | | | |
| Device | H1-230 | H2-712 | H1-231 | 3.1 | 36.4 | Red | 167 |
| Example 7 | | | | | | | |
| Device | H1-233 | H2-751 | H1-234 | 3.1 | 35.6 | Red | 140 |
| Example 8 | | | | | | | |
| Device | H1-232 | H2-146 | H1-235 | 3.2 | 36.5 | Red | 188 |
| Example 9 | | | | | | | |
| Device | H1-200 | H2-858 | H2-859 | 3.0 | 35.7 | Red | 164 |
| Example 10 | | | | | | | |
| Device | H1-234 | H2-746 | H2-860 | 3.0 | 36.5 | Red | 184 |
| Example 11 | | | | | | | |
| Device | H1-200 | H2-858 | H2-861 | 3.0 | 35.5 | Red | 190 |
| Example 12 | | | | | | | |
| Device | H1-21 | H2-864 | H2-332 | 2.9 | 31.3 | Red | 84 |
| Example 13 | | | | | | | |
| Device | H1-282 | H2-864 | H2-332 | 3.0 | 33.9 | Red | 196 |
| Example 14 | | | | | | | |
| Device | H1-227 | H2-746 | H1-185 | 3.1 | 34.5 | Red | 178 |
| Example 15 | | | | | | | |
| Comparative | H1-222 | — | — | 4.2 | 9.3 | Red | 4.2 |
| Example 1 | | | | | | | |
| Comparative | — | — | H1-221 | 3.8 | 7.5 | Red | 2.0 |
| Example 2 | | | | | | | |
| Comparative | — | H2-745 | | 3.5 | 28.8 | Red | 13.7 |
| Example 3 | | | | | | | |
| Comparative | H1-226 | — | — | 3.7 | 10.2 | Red | 1.6 |
| Example 4 | | | | | | | |
| Comparative | H1-227 | — | — | 4.1 | 9.6 | Red | 4.3 |
| Example 5 | | | | | | | |
| Comparative | — | — | H1-228 | 3.8 | 10.3 | Red | 2.4 |
| Example 6 | | | | | | | |
| Comparative | — | — | H1-229 | 4.5 | 8.1 | Red | 6.2 |
| Example 7 | | | | | | | |
| Comparative | — | H2-746 | — | 3.0 | 24.6 | Red | 15.3 |
| Example 8 | | | | | | | |
| Comparative | — | H2-747 | — | 3.1 | 24.6 | Red | 8.1 |
| Example 9 | | | | | | | |
| Comparative | H1-230 | — | — | 4.3 | 6.5 | Red | 2.0 |
| Example 10 | | | | | | | |
| Comparative | — | — | H1-225 | 3.7 | 9.5 | Red | 3.3 |
| Example 11 | | | | | | | |
| Comparative | — | — | H1-231 | 4.7 | 8.0 | Red | 2.0 |
| Example 12 | | | | | | | |
| Comparative | — | H2-712 | — | 3.6 | 30.4 | Red | 11.3 |
| Example 13 | | | | | | | |
| Comparative | H1-232 | — | — | 4.3 | 7.8 | Red | 2.3 |
| Example 14 | | | | | | | |
| Comparative | — | H2-751 | — | 3.6 | 29.4 | Red | 9.9 |
| Example 15 | | | | | | | |
| Comparative | — | H2-146 | — | 3.5 | 31.8 | Red | 17.5 |
| Example 16 | | | | | | | |
| Comparative | — | — | H1-234 | 4.3 | 7.4 | Red | 3.4 |
| Example 17 | | | | | | | |
| Comparative | — | H2-858 | — | 3.7 | 3.7 | Red | 30 |
| Example 18 | | | | | | | |
| Comparative | — | H2-860 | — | 3.5 | 31.5 | Red | 23 |
| Example 19 | | | | | | | |
| Comparative | — | H2-859 | — | 3.2 | 30.1 | Red | 6 |
| Example 20 | | | | | | | |
| Comparative | — | H2-861 | — | 3.2 | 29.0 | Red | 0 |
| Example 21 | | | | | | | |
| Comparative | — | H2-863 | — | 3.6 | 29.9 | Red | 24 |
| Example 22 | | | | | | | |
| Comparative | H1-21 | — | — | 6.1 | 3.3 | Red | 1.7 |
| Example 23 | | | | | | | |
| Comparative | H1-282 | — | — | 4.3 | 7.8 | Red | 7.2 |
| Example 24 | | | | | | | |
| Comparative | — | H2-864 | — | 3.7 | 28.0 | Red | 9.0 |
| Example 25 | | | | | | | |
| Comparative | — | — | H2-332 | 3.1 | 26.6 | Red | 6.7 |
| Example 26 | | | | | | | |
| Comparative | — | — | H1-185 | 4.4 | 9.2 | Red | 8.3 |
| Example 27 | | | | | | | |

From Table 1 above, it can be confirmed that the OLED comprising the compound according to the present disclosure as the three types of host materials exhibits lower driving voltage and/or higher luminous efficiency, in particular, significantly improved lifetime properties, compared to the conventional OLED using a single host material (Comparative Examples 1 to 27).

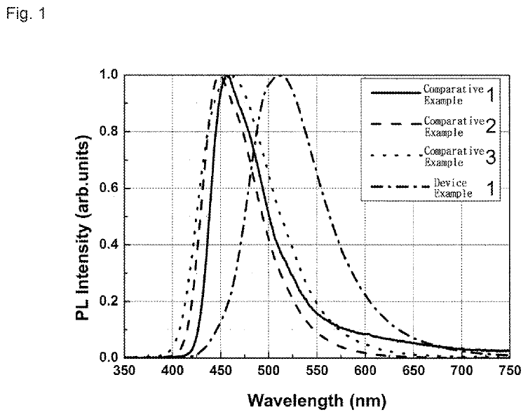

FIG. 1 illustrates a graph showing luminous properties according to wavelengths of the host material of the combination of three host compounds according to Device Example 1, and the host material according to Comparative Examples 1 to 3. The wavelength values corresponding to PL max are described in Table 2 below.

TABLE 2

| | Host | PL max |
|---|---|---|
| Comparative | H1-222 | 450 |
| Example 1 | | |
| Comparative | H1-221 | 456 |
| Example 2 | | |
| Comparative | H2-745 | 461 |
| Example 3 | | |
| Device Example 1 | H1-222:H2-745:H1-221 | 513 |
| | (2.5:5.0:2.5, wt %) | |

With Reference to the results of FIG. 1 and Table 2, it can be seen that the combination of the organic compounds according to Device Example 1 exhibits relatively long wavelength properties compared to the organic materials according to Comparative Examples 1 to 3, which is evidence of the formation of an exciplex of the organic light-emitting material. In addition, it can be confirmed that a PL peak in a new wavelength band is formed due to a combination of three types of hosts.

That is, without being limited by theory, the three types of host materials comprising a specific combination of the compounds of the present disclosure can facilitate the control of the energy level and mobility of the phosphorescent host material to improve the charge balance in the light-emitting layer, and thereby an organic electroluminescent device having high luminous efficiency and/or improved lifetime properties can be provided.

The compounds used in the Device Examples and the Comparative Examples are shown in Table 3 below.

TABLE 3

| | |
|---|---|
| Hole Injection Layer/ Hole Transport Layer | 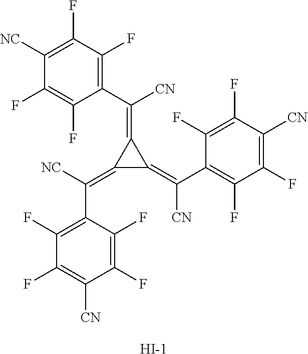 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 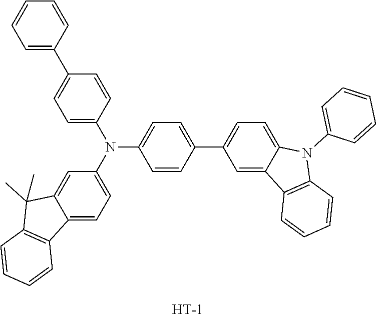 |
| | 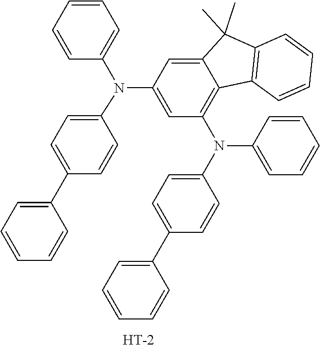 |
| Light Emitting Layer | 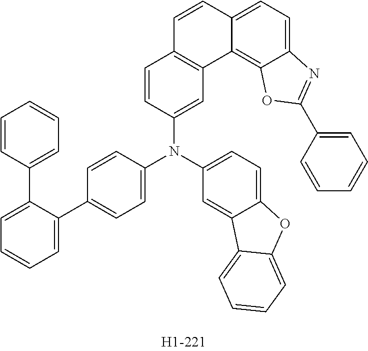 [SMILES: c1ccc(-c2nc3ccc4ccc5ccc(N(c6ccc(-c7ccccc7-c7ccccc7)cc6)c6ccc7oc8ccccc8c7c6)cc5c4c3o2)cc1] |
| | 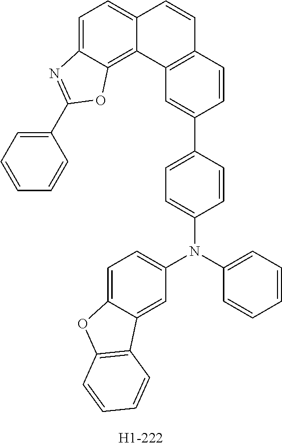 [SMILES: c1ccc(-c2nc3ccc4ccc5ccc(-c6ccc(N(c7ccccc7)c7ccc8oc9ccccc9c8c7)cc6)cc5c4c3o2)cc1] |
| | 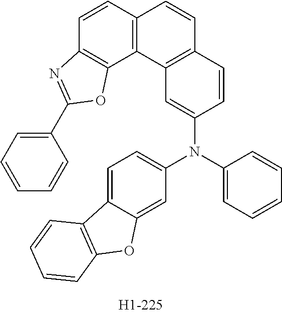 [SMILES: c1ccc(-c2nc3ccc4ccc5ccc(N(c6ccccc6)c6ccc7c(c6)oc6ccccc67)cc5c4c3o2)cc1] |
| | 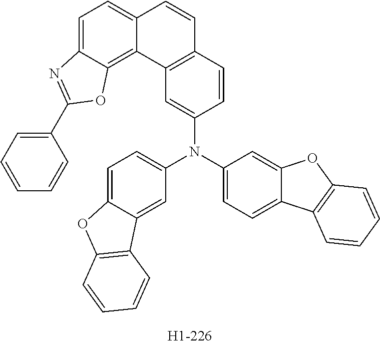 [SMILES: c1ccc(-c2nc3ccc4ccc5ccc(N(c6ccc7c(c6)oc6ccccc67)c6ccc7oc8ccccc8c7c6)cc5c4c3o2)cc1] |
| | 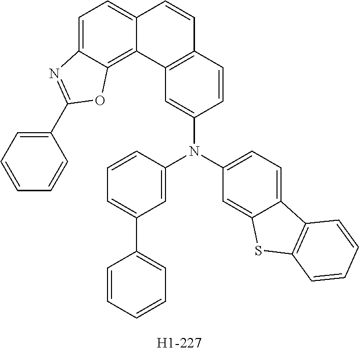 [SMILES: c1ccc(-c2cccc(N(c3ccc4c(c3)sc3ccccc34)c3ccc4ccc5ccc6nc(-c7ccccc7)oc6c5c4c3)c2)cc1] |
| | 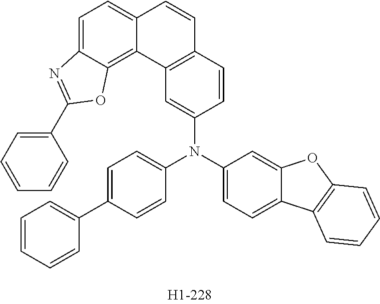 [SMILES: c1ccc(-c2ccc(N(c3ccc4c(c3)oc3ccccc34)c3ccc4ccc5ccc6nc(-c7ccccc7)oc6c5c4c3)cc2)cc1] |
| | 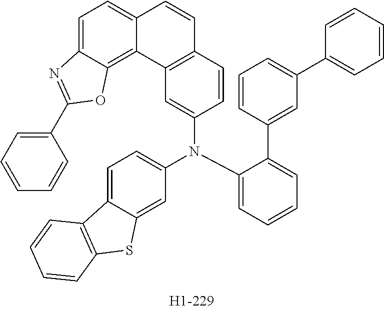 [SMILES: c1ccc(-c2cccc(-c3ccccc3N(c3ccc4c(c3)sc3ccccc34)c3ccc4ccc5ccc6nc(-c7ccccc7)oc6c5c4c3)c2)cc1] |
| | 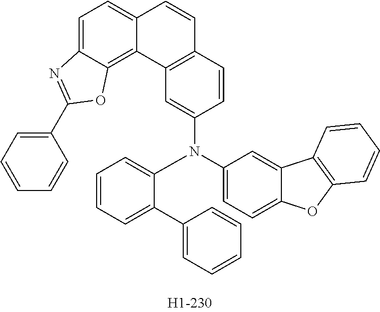 [SMILES: c1ccc(-c2nc3ccc4ccc5ccc(N(c6ccc7oc8ccccc8c7c6)c6ccccc6-c6ccccc6)cc5c4c3o2)cc1] |
| | 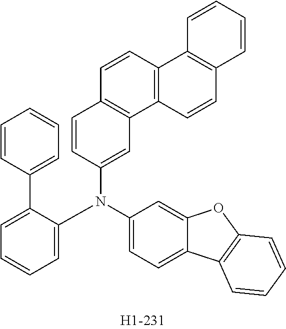 [SMILES: c1ccc(-c2ccccc2N(c2ccc3c(c2)oc2ccccc23)c2ccc3ccc4c5ccccc5ccc4c3c2)cc1] |
| | 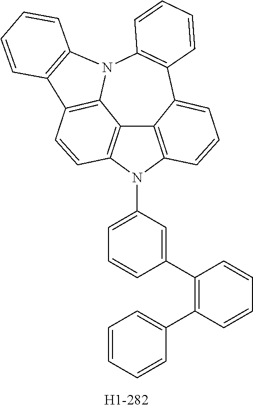 [SMILES: c1ccc(-c2ccccc2-c2cccc(-n3c4cccc5c6ccccc6n6c7ccccc7c7ccc3c(c54)c76)c2)cc1] |
| | 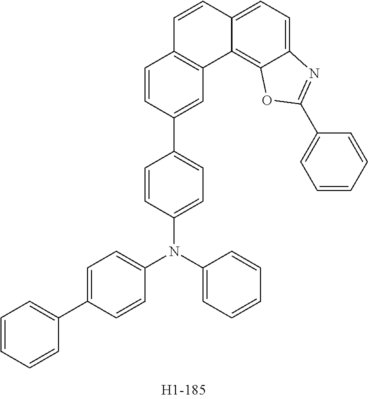 [SMILES: c1ccc(-c2ccc(N(c3ccccc3)c3ccc(-c4ccc5ccc6ccc7nc(-c8ccccc8)oc7c6c5c4)cc3)cc2)cc1] |
| | 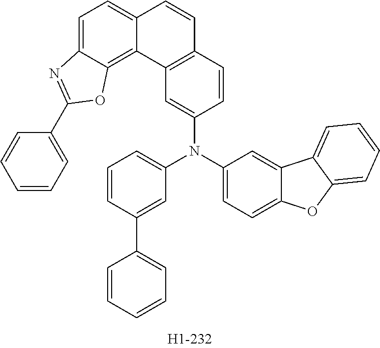 [SMILES: c1ccc(-c2cccc(N(c3ccc4oc5ccccc5c4c3)c3ccc4ccc5ccc6nc(-c7ccccc7)oc6c5c4c3)c2)cc1] |
| | 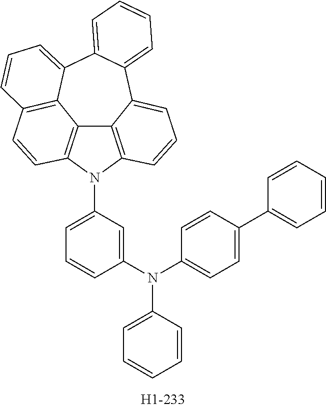 [SMILES: c1ccc(-c2ccc(N(c3ccccc3)c3cccc(-n4c5cccc6c5c5c7c(cccc7ccc54)-c4ccccc4-6)c3)cc2)cc1] |
| | 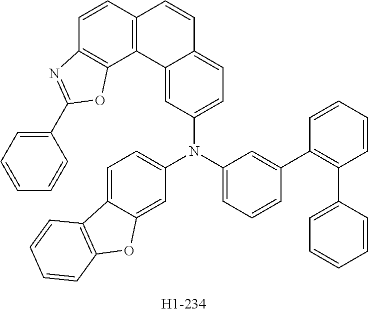 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | 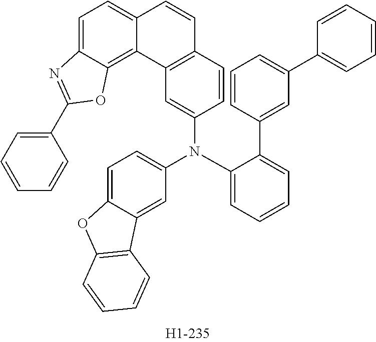 [SMILES: c1ccc(-c2cccc(-c3ccccc3N(c3ccc4oc5ccccc5c4c3)c3ccc4ccc5ccc6nc(-c7ccccc7)oc6c5c4c3)c2)cc1] |
| | 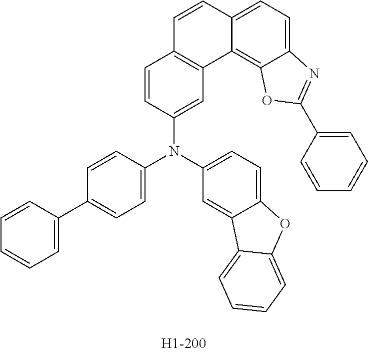 [SMILES: c1ccc(-c2ccc(N(c3ccc4oc5ccccc5c4c3)c3ccc4ccc5ccc6nc(-c7ccccc7)oc6c5c4c3)cc2)cc1] |
| | 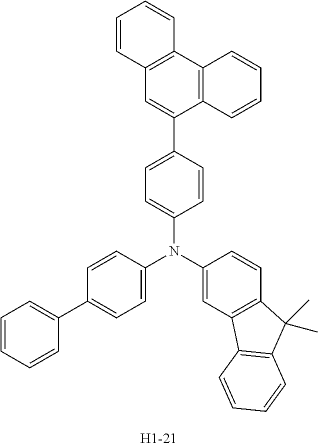 [SMILES: CC1(C)c2ccccc2-c2cc(N(c3ccc(-c4ccccc4)cc3)c3ccc(-c4cc5ccccc5c5ccccc45)cc3)ccc21] |
| | 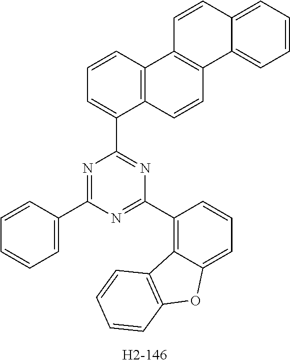 [SMILES: c1ccc(-c2nc(-c3cccc4c3ccc3c5ccccc5ccc43)nc(-c3cccc4oc5ccccc5c34)n2)cc1] |
| | 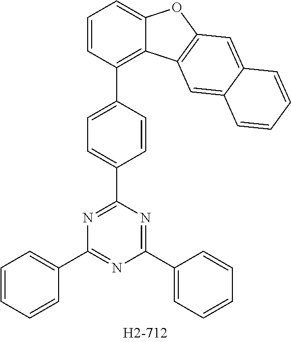 [SMILES: c1ccc(-c2nc(-c3ccccc3)nc(-c3ccc(-c4cccc5oc6cc7ccccc7cc6c45)cc3)n2)cc1] |
| | 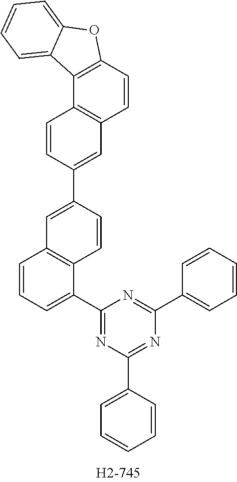 [SMILES: c1ccc(-c2nc(-c3ccccc3)nc(-c3cccc4cc(-c5ccc6c(ccc7oc8ccccc8c76)c5)ccc34)n2)cc1] |
| | 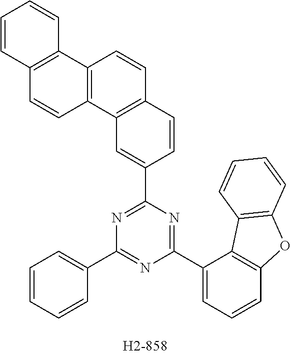 [SMILES: c1ccc(-c2nc(-c3ccc4ccc5c6ccccc6ccc5c4c3)nc(-c3cccc4oc5ccccc5c34)n2)cc1] |
| | 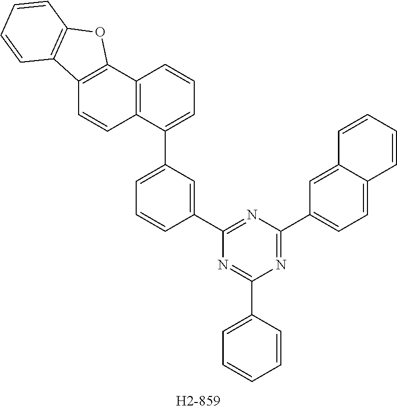 [SMILES: c1ccc(-c2nc(-c3cccc(-c4cccc5c4ccc4c6ccccc6oc54)c3)nc(-c3ccc4ccccc4c3)n2)cc1] |
| | 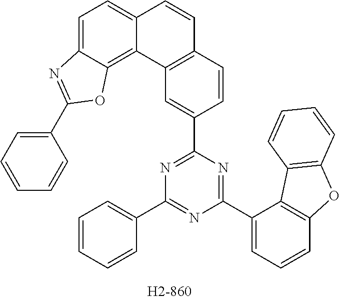 [SMILES: c1ccc(-c2nc(-c3ccc4ccc5ccc6nc(-c7ccccc7)oc6c5c4c3)nc(-c3cccc4oc5ccccc5c34)n2)cc1] |
| | 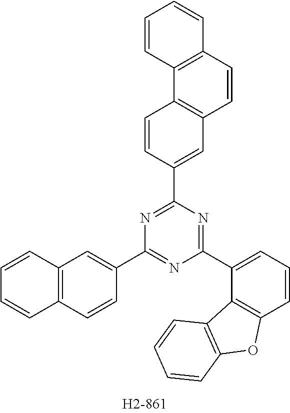 [SMILES: c1ccc2cc(-c3nc(-c4ccc5c(ccc6ccccc65)c4)nc(-c4cccc5oc6ccccc6c45)n3)ccc2c1] |
| | 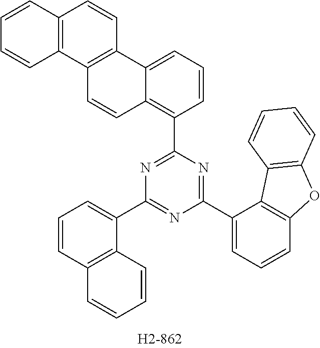 [SMILES: c1ccc2c(-c3nc(-c4cccc5c4ccc4c6ccccc6ccc54)nc(-c4cccc5oc6ccccc6c45)n3)cccc2c1] |
| | 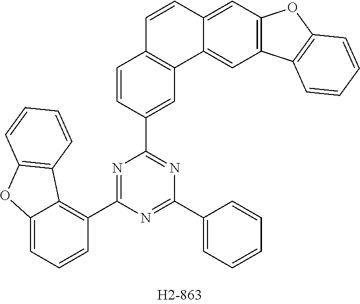 [SMILES: c1ccc(-c2nc(-c3ccc4ccc5cc6oc7ccccc7c6cc5c4c3)nc(-c3cccc4oc5ccccc5c34)n2)cc1] |
| | 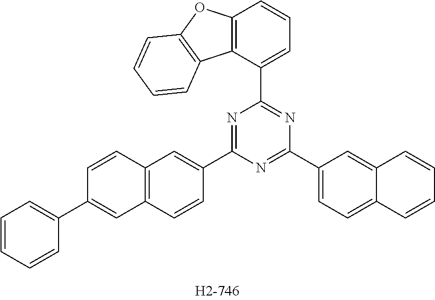 [SMILES: c1ccc(-c2ccc3cc(-c4nc(-c5ccc6ccccc6c5)nc(-c5cccc6oc7ccccc7c56)n4)ccc3c2)cc1] |
| | 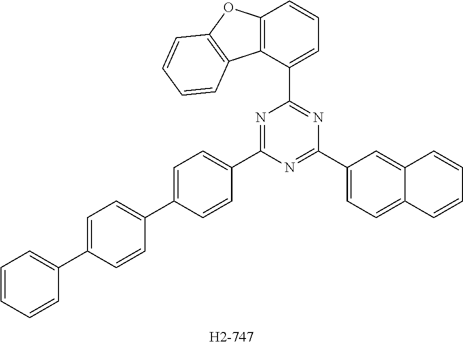 [SMILES: c1ccc(-c2ccc(-c3ccc(-c4nc(-c5ccc6ccccc6c5)nc(-c5cccc6oc7ccccc7c56)n4)cc3)cc2)cc1] |
| | 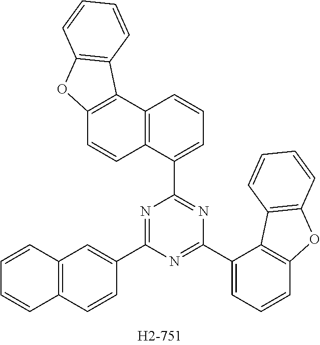 [SMILES: c1ccc2cc(-c3nc(-c4cccc5c4ccc4oc6ccccc6c45)nc(-c4cccc5oc6ccccc6c45)n3)ccc2c1] |
| | 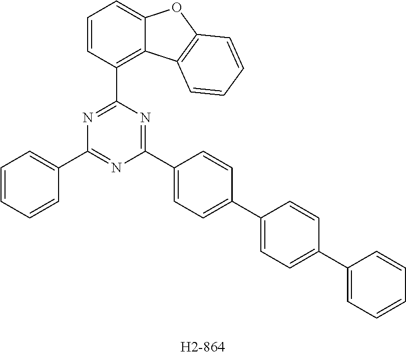 [SMILES: c1ccc(-c2ccc(-c3ccc(-c4nc(-c5ccccc5)nc(-c5cccc6oc7ccccc7c56)n4)cc3)cc2)cc1] |
| | 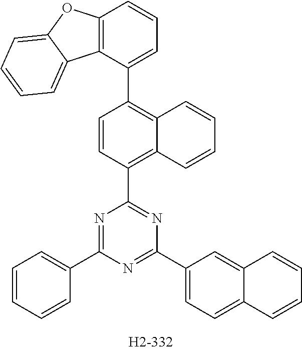 [SMILES: c1ccc(-c2nc(-c3ccc4ccccc4c3)nc(-c3ccc(-c4cccc5oc6ccccc6c45)c4ccccc34)n2)cc1] |
| | 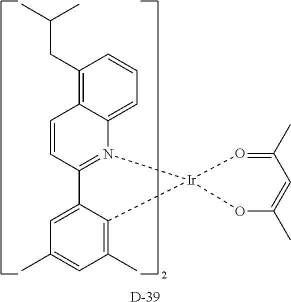 |
| Electron Transport Layer/ Electron Injection Layer | 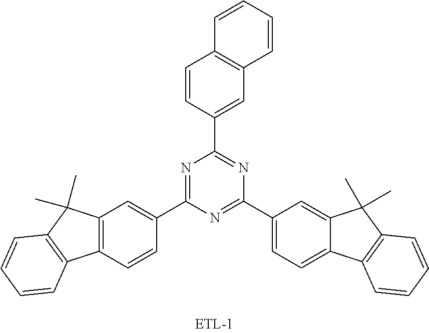 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 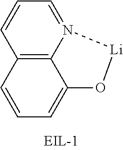 |

1. A plurality of host materials comprising a first host material, a second host material, and a third host material; wherein each of the first host material, the second host material, and the third host material does not comprise a carbazole or fused carbazole structure; wherein
the first host material, the second host material, and the third host material are different from each other; wherein
the first host material consists of a substituted or unsubstituted benzophenanthrofuranyl or a substituted or unsubstituted benzophenanthrofuranyl; and wherein
the maximum emission wavelength of the plurality of host materials is shifted by at least 20 nm from the maximum emission wavelength of each of the first host material, the second host material, and the third host material.

2. The plurality of host materials according to claim 1, wherein the first host material comprises a compound represented by the following formula 1, the second host material comprises a compound represented by any one of the following formulas 2 to 4, and the third host material comprises a compound represented by any one of the following formulas 1 to 4, wherein the third host material is different from the first host material and the second host material: 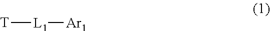
in formula 1,
T represents any one selected from the group consisting of the following formulas 1-1 to 1-5: 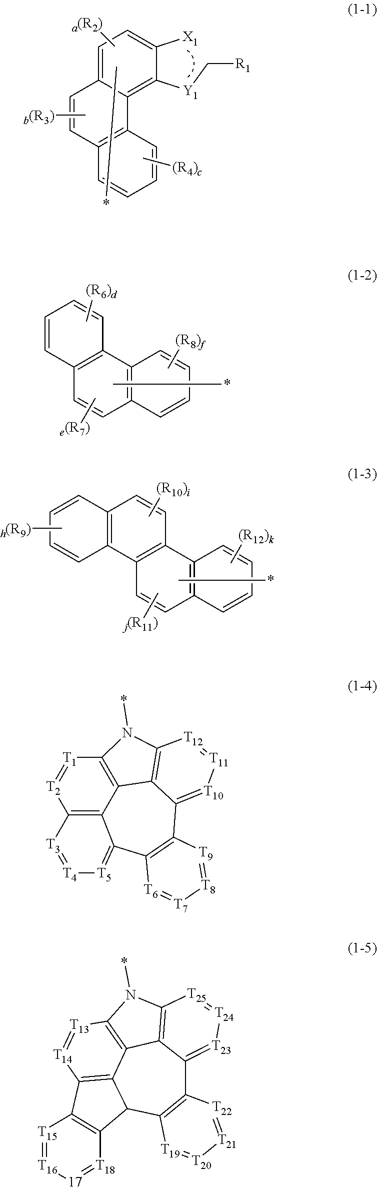
wherein,
X₁ and Y₁, each independently, represent —N═, —NR₅—, —O—, or —S—, with the proviso that any one of X₁ and Y₁ represents —N═, and the other of X₁ and Y₁ represents —NR₅—, —O—, or —S—;
R₁ represents a substituted or unsubstituted (C6-C30)aryl, or a substituted or unsubstituted (3- to 30-membered)heteroaryl;
R₂ to R₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C6-C30)aryl(3- to 30-membered)heteroarylamino, or a substituted or unsubstituted (C1-C30)alkyl(C6-C30)arylamino; or may be linked to an adjacent substituent(s) to form a ring(s);
a and b, each independently, represent 1 or 2; c represents an integer of 1 to 4; where if each of a to c is an integer of 2 or more, each of R₂ to each of R₄ may be the same or different;
R₆ to R₁₂, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
T₁ to T₂₅, each independently, represent N or CV₁;
V₁, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent(s) to form a ring(s);
d, f, h, and k, each independently, represent an integer of 1 to 4; e, i, and j, each independently, represent an integer of 1 or 2; where if each of d to f and h to k is an integer of 2 or more, each of R₆ to each of R₁₁ may be the same or different;
Ar₁ represents a substituted or unsubstituted (C6-C30)aryl, a substituted or unsubstituted (3- to 30-membered)heteroaryl, or 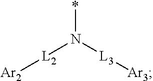
Ar₂ and Ar₃, each independently, represent a substituted or unsubstituted (C6-C30)aryl, a substituted or unsubstituted (3- to 30-membered)heteroaryl, or a substituted or unsubstituted (C3-C30)cycloalkyl; and
L₁ to L₃, each independently, represent a single bond, a substituted or unsubstituted (C6-C30)arylene, or a substituted or unsubstituted (3- to 30-membered)heteroarylene; 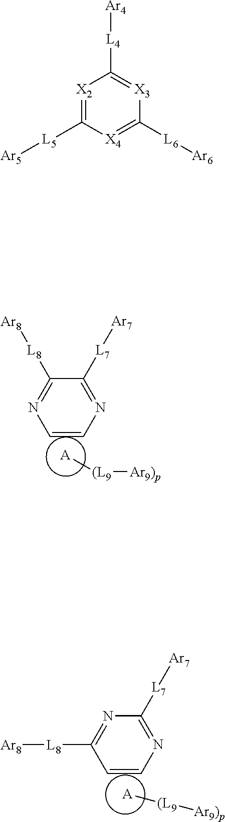
in formulas 2 to 4,
X₂ to X₄, each independently, represent CR₁₃ or N, with the proviso that at least one of X₂ to X₄ represents N;
R₁₃, each independently, represents hydrogen, deuterium, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; or may be linked to an adjacent substituent(s) to form a ring(s);
L₄ to L₉, each independently, represent a single bond, a substituted or unsubstituted (C6-C30)arylene, or a substituted or unsubstituted (3- to 30-membered)heteroarylene;
ring A represents a benzene ring or a naphthalene ring;
Ar₄ to Ar₉, each independently, represent a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; or may be linked to an adjacent substituent(s) to form a ring(s); and
p represents an integer of 0 to 6; where if p is an integer of 2 or more, each of L₉ and each of Ar₉ may be the same or different.

3. The plurality of host materials according to claim 2, wherein the substituent(s) of the substituted alkyl, the substituted cycloalkyl, the substituted heterocycloalkyl, the substituted fused ring group of a aliphatic ring(s) and a aromatic ring(s), the substituted aryl, the substituted arylene, the substituted heteroaryl, the substituted heteroarylene, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-alkenylamino, the substituted alkylalkenylamino, the substituted mono- or di-arylamino, the substituted alkylarylamino, the substituted mono- or di-heteroarylamino, the substituted alkylheteroarylamino, the substituted alkenylarylamino, the substituted alkenylheteroarylamino, and the substituted arylheteroarylamino, each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl unsubstituted or substituted with a (C6-C30)aryl(s); a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

4. The plurality of host materials according to claim 2, wherein the formula 1 is represented by any one of the following formulas 1-11 to 1-16: 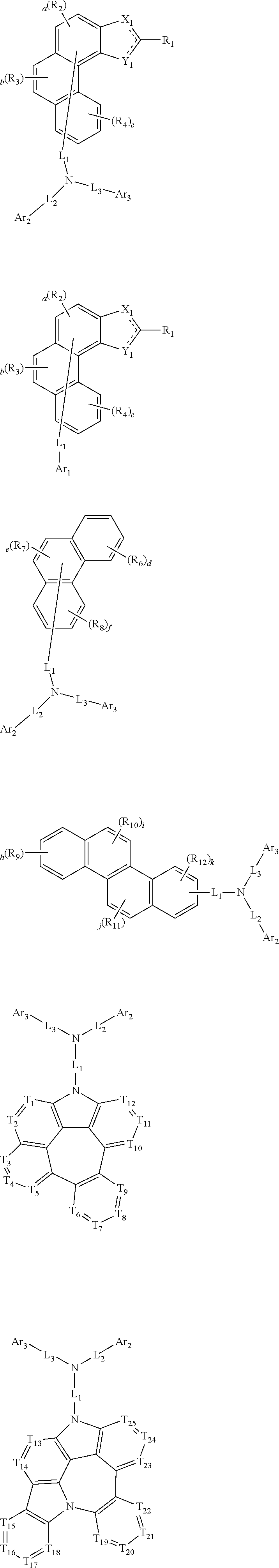
in formulas 1-11 to 1-16,
Ar₁ represents a substituted or unsubstituted (C6-C30)aryl, or a substituted or unsubstituted (3- to 30-membered)heteroaryl; and
X₁, Y₁, R₁ to R₄, R₆ to R₁₂, T₁ to T₂₅, L₁ to L₃, Ar₂, Ar₃, a to f, and h to k are as defined in claim 2.

5. The plurality of host materials according to claim 2, wherein in formulas 2 to 4, at least one of Ar₄ to Ar₉ is selected from the group consisting of the following formulas a-1 to a-7: 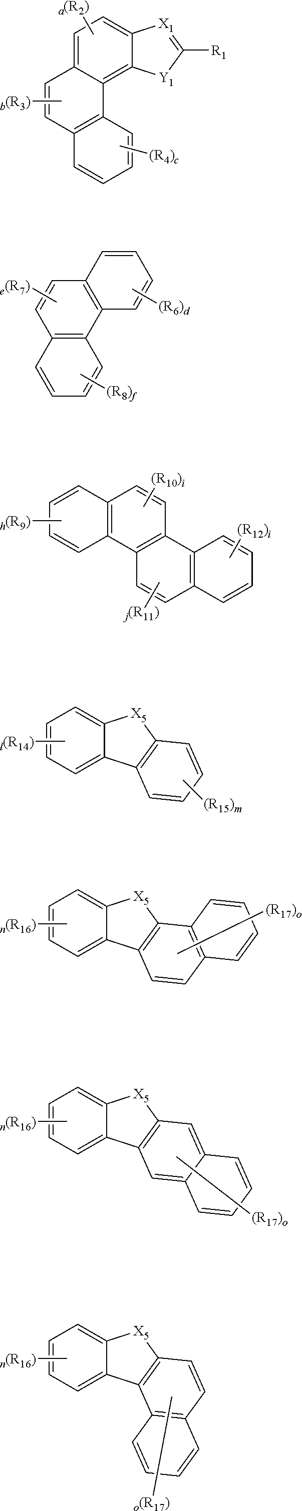
in formulas a-1 to a-7,
X₅, each independently, represents 0, S, or CR₁₈R₁₉;
R₁₈ and R₁₉, each independently, represent a substituted or unsubstituted (C1-C30)alkyl, a substituted or unsubstituted (C3-C30)cycloalkyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; or R₁₈ and R₁₉ may be linked to each other to form a spiro ring;
R₁₄ and R₁₅, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
l to n, each independently, represent an integer of 1 to 4; o represents an integer of 1 to 6; where if each of l to o is an integer of 2 or more, each of R₁₄ to each of R₁₇ may be the same or different;
R₆ to R₈, R₁₆, and R₁₇, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent(s) to form a ring(s); and
X₁, Y₁, R₁ to R₄, R₉ to R₁₂, a to f, and h to k are as defined in claim 2.

6. The plurality of host materials according to claim 2, wherein any two of the first host material, the second host material and the third host material have a difference in deposition temperature of between 0° C. and 20° C. at 10⁻³ torr or less.

7. The plurality of host materials according to claim 2, wherein each of the first host material and the third host material has stronger hole transport properties than the second host material; and the HOMO energy level of a light-emitting layer comprising the plurality of host materials satisfies the following Equation 1 or 2:
$$\vert {A}_{h 1} - {A}_{h 3} \vert \leq 0.5 eV \qquad [ Equation 1 ]$$
$$\vert {A}_{h 1} - {A}_{h 3} \vert \leq 0.3 eV \qquad [ Equation 2 ]$$
in Equations 1 and 2, Aₕ₁ represents the HOMO energy level of the first host material, and Aₕ₃ represents the HOMO energy level of the third host material.

8. The plurality of host materials according to claim 2, wherein each of the second host material and the third host material has stronger electron transport properties than the first host material; and the LUMO energy level of a light-emitting layer comprising the plurality of host materials satisfies the following Equation 3 or 4:
$$\vert {A}_{12} - {A}_{13} \vert \leq 0.5 eV \qquad [ Equation 3 ]$$
$$\vert {A}_{12} - {A}_{13} \vert \leq 0.3 eV \qquad [ Equation 4 ]$$
in Equations 3 and 4, Aₗ₂ represents the LUMO energy level of the second host material, and Aₗ₃ represents the LUMO energy level of the third host material.

9. The plurality of host materials according to claim 2, wherein the plurality of host materials comprise 5 wt % to 70 wt % of the first host material, 5 wt % to 70 wt % of the second host material, and 10 wt % to 90 wt % of the third host material.

10. The plurality of host materials according to claim 2, wherein the compound represented by formula 1 is at least one selected from the group consisting of the following compounds:

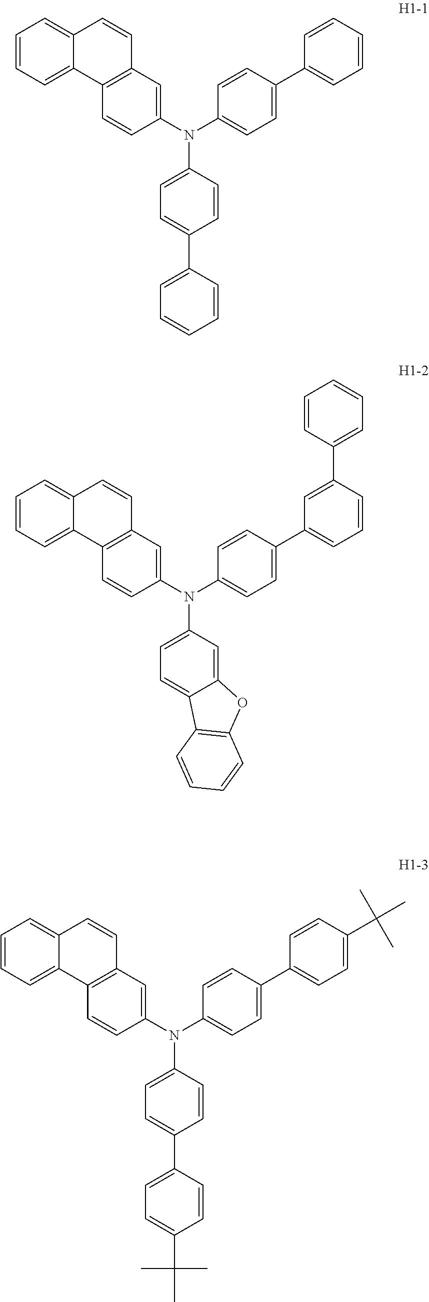 [SMILES: c1ccc(-c2ccc(N(c3ccc(-c4ccccc4)cc3)c3ccc4c(ccc5ccccc54)c3)cc2)cc1 ; c1ccc(-c2cccc(-c3ccc(N(c4ccc5c(ccc6ccccc65)c4)c4ccc5c(c4)oc4ccccc45)cc3)c2)cc1 ; CC(C)(C)c1ccc(-c2ccc(N(c3ccc(-c4ccc(C(C)(C)C)cc4)cc3)c3ccc4c(ccc5ccccc54)c3)cc2)cc1]

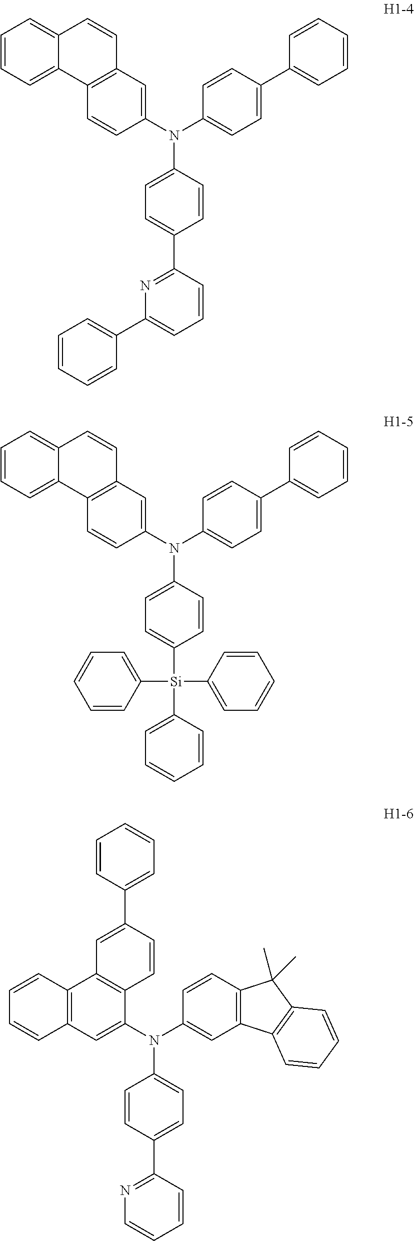

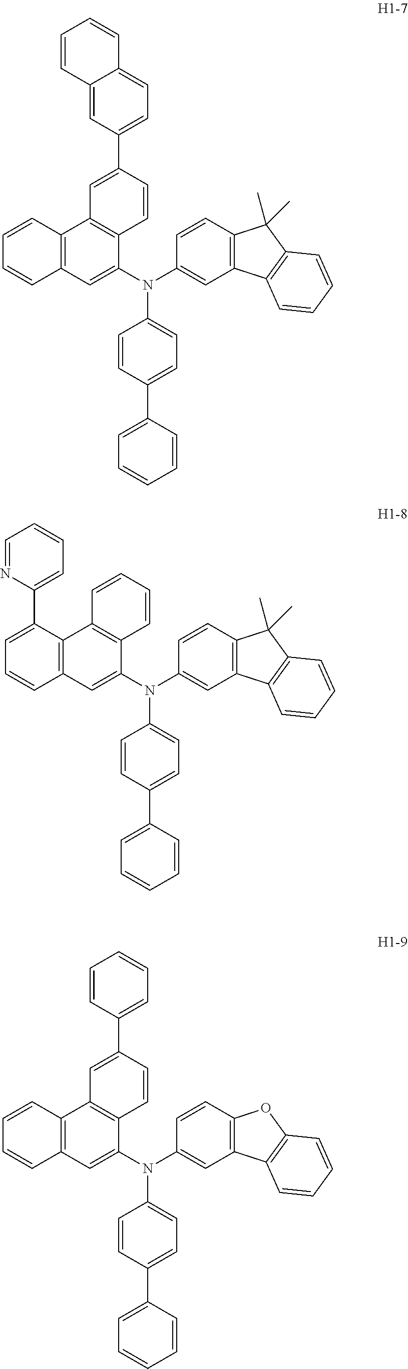 [SMILES: CC1(C)c2ccccc2-c2cc(N(c3ccc(-c4ccccc4)cc3)c3cc4ccccc4c4cc(-c5ccc6ccccc6c5)ccc34)ccc21 ; CC1(C)c2ccccc2-c2cc(N(c3ccc(-c4ccccc4)cc3)c3cc4cccc(-c5ccccn5)c4c4ccccc34)ccc21 ; c1ccc(-c2ccc(N(c3ccc4oc5ccccc5c4c3)c3cc4ccccc4c4cc(-c5ccccc5)ccc34)cc2)cc1]

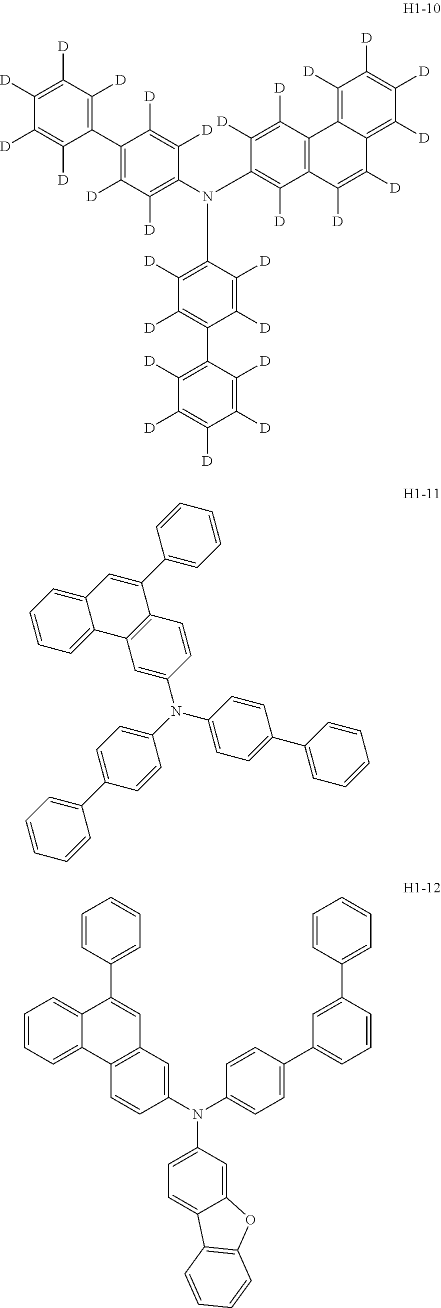 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c([2H])c([2H])c([2H])c([2H])c34)c([2H])c2[2H])c([2H])c1[2H] ; c1ccc(-c2ccc(N(c3ccc(-c4ccccc4)cc3)c3ccc4c(-c5ccccc5)cc5ccccc5c4c3)cc2)cc1 ; c1ccc(-c2cccc(-c3ccc(N(c4ccc5c(c4)cc(-c4ccccc4)c4ccccc45)c4ccc5c(c4)oc4ccccc45)cc3)c2)cc1]

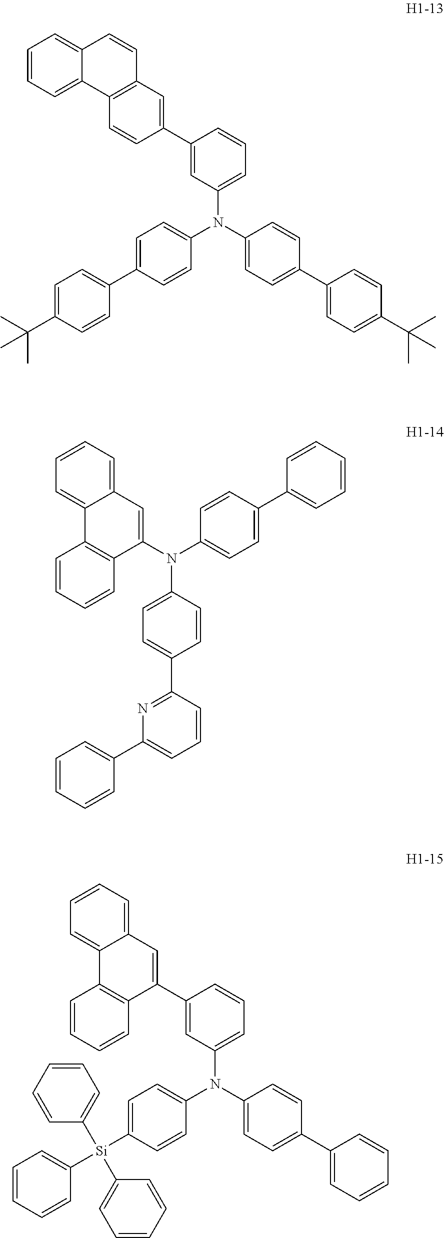 [SMILES: CC(C)(C)c1ccc(-c2ccc(N(c3ccc(-c4ccc(C(C)(C)C)cc4)cc3)c3cccc(-c4ccc5c(ccc6ccccc65)c4)c3)cc2)cc1 ; c1ccc(-c2ccc(N(c3ccc(-c4cccc(-c5ccccc5)n4)cc3)c3cc4ccccc4c4ccccc34)cc2)cc1 ; c1ccc(-c2ccc(N(c3ccc([Si](c4ccccc4)(c4ccccc4)c4ccccc4)cc3)c3cccc(-c4cc5ccccc5c5ccccc45)c3)cc2)cc1]

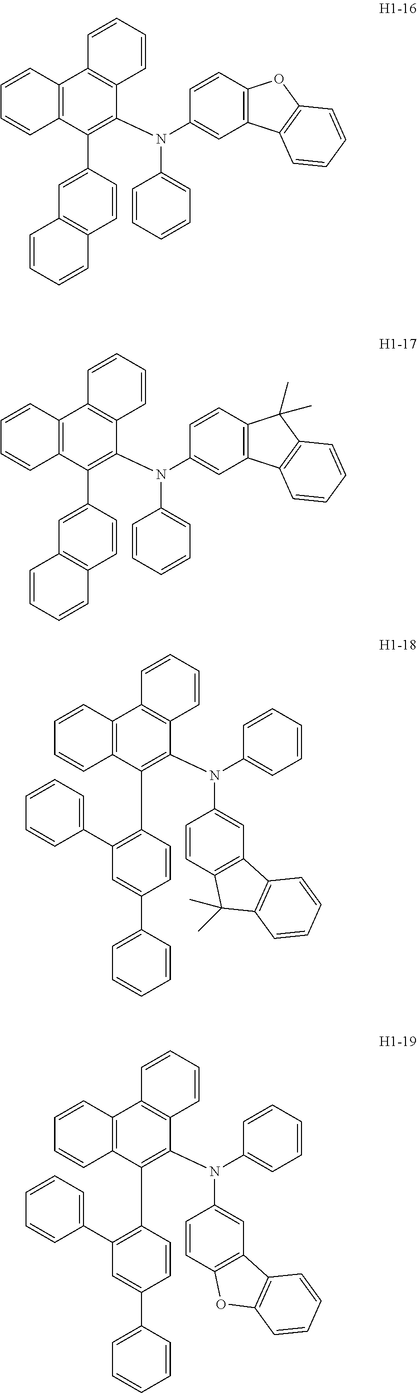 [SMILES: c1ccc(N(c2ccc3oc4ccccc4c3c2)c2c(-c3ccc4ccccc4c3)c3ccccc3c3ccccc23)cc1 ; CC1(C)c2ccccc2-c2cc(N(c3ccccc3)c3c(-c4ccc5ccccc5c4)c4ccccc4c4ccccc34)ccc21 ; CC1(C)c2ccccc2-c2cc(N(c3ccccc3)c3c(-c4ccc(-c5ccccc5)cc4-c4ccccc4)c4ccccc4c4ccccc34)ccc21 ; c1ccc(-c2ccc(-c3c(N(c4ccccc4)c4ccc5oc6ccccc6c5c4)c4ccccc4c4ccccc34)c(-c3ccccc3)c2)cc1]

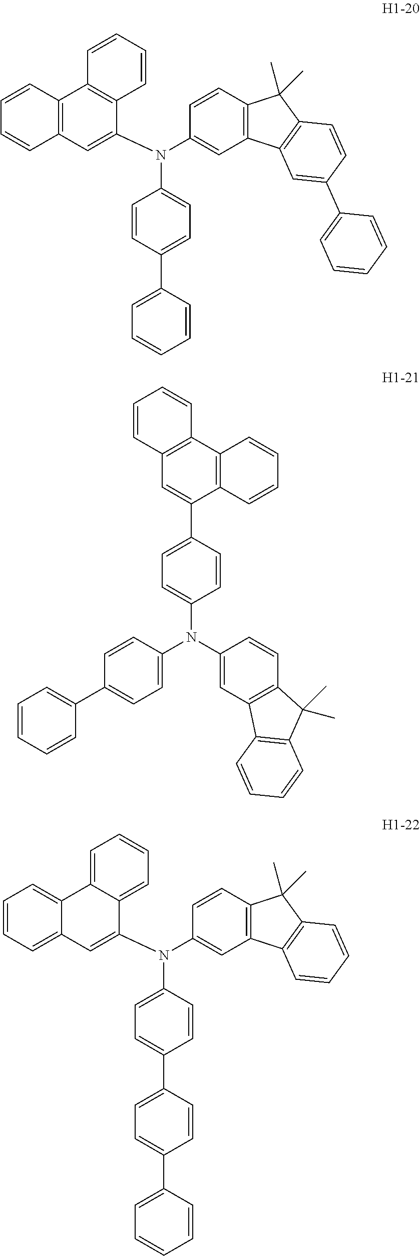

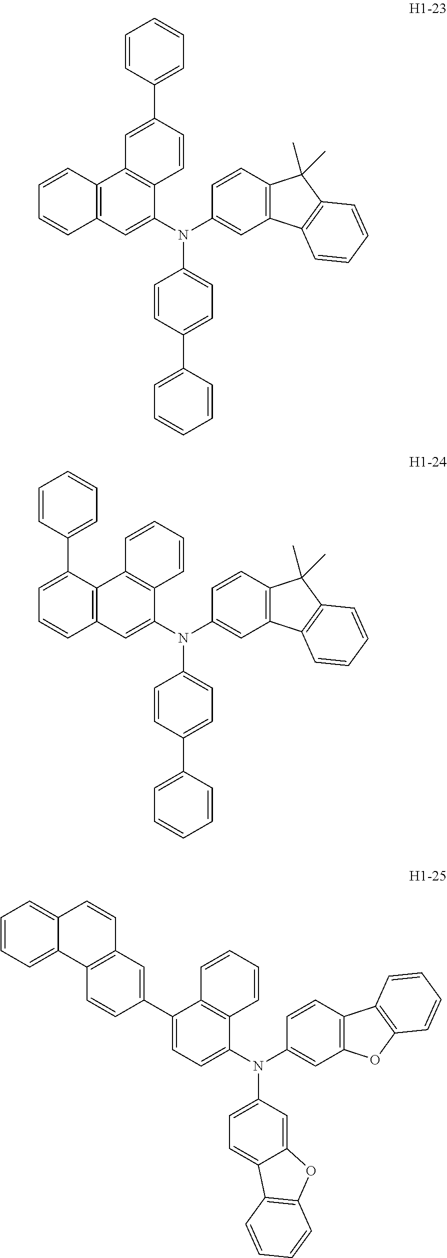 [SMILES: CC1(C)c2ccccc2-c2cc(N(c3ccc(-c4ccccc4)cc3)c3cc4ccccc4c4cc(-c5ccccc5)ccc34)ccc21 ; CC1(C)c2ccccc2-c2cc(N(c3ccc(-c4ccccc4)cc3)c3cc4cccc(-c5ccccc5)c4c4ccccc34)ccc21 ; c1ccc2c(c1)ccc1cc(-c3ccc(N(c4ccc5c(c4)oc4ccccc45)c4ccc5c(c4)oc4ccccc45)c4ccccc34)ccc12]

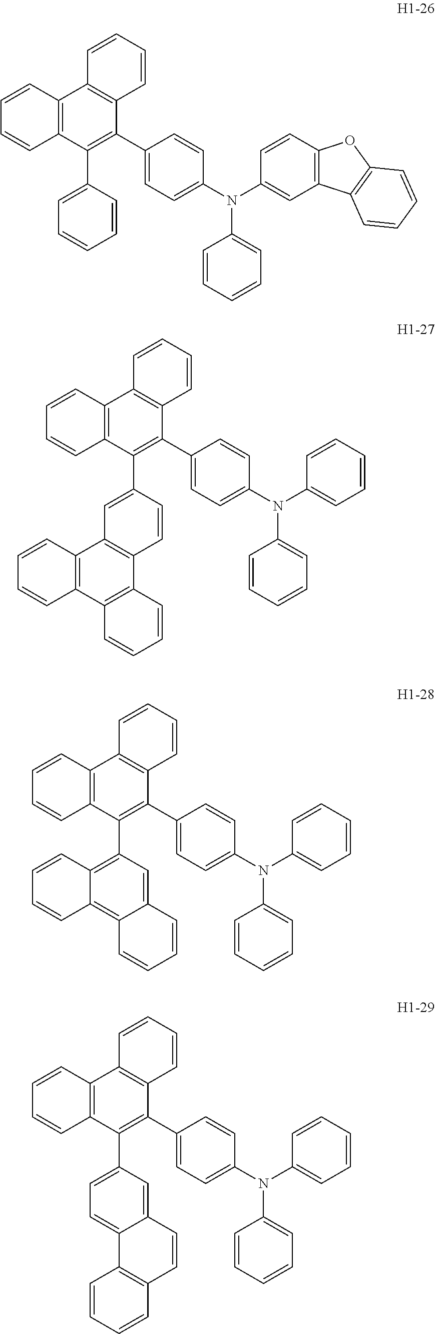

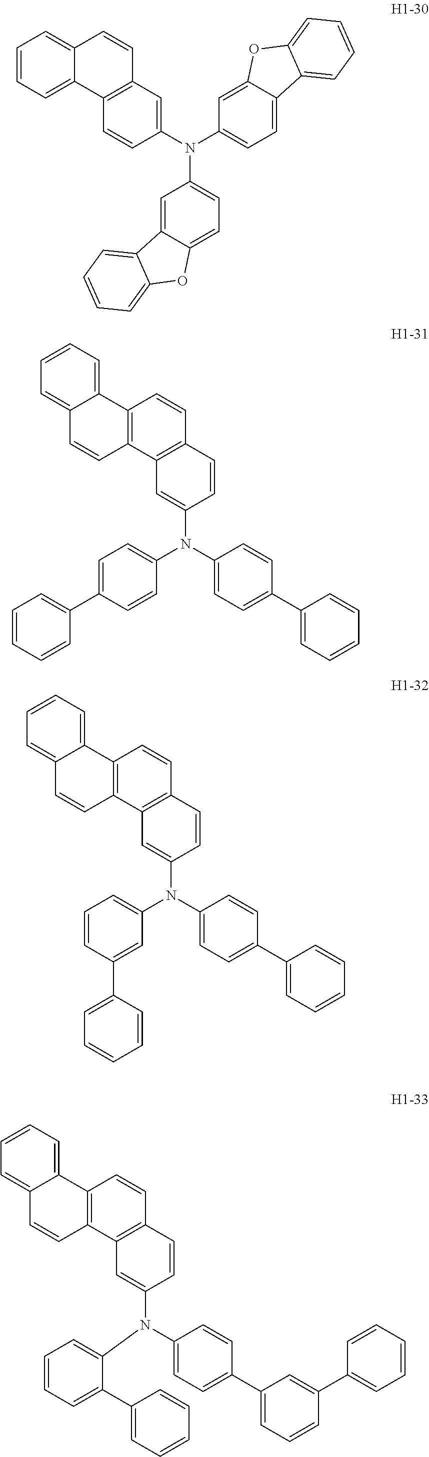

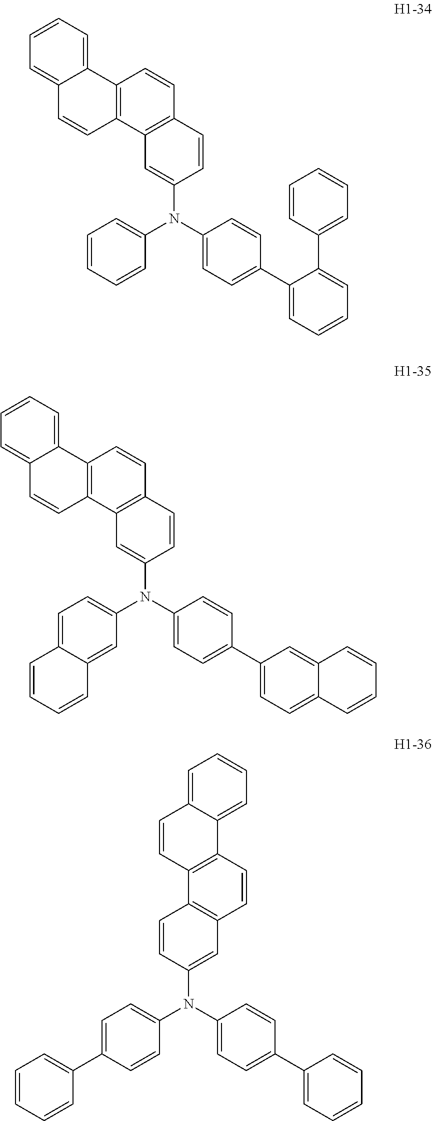 [SMILES: c1ccc(-c2ccccc2-c2ccc(N(c3ccccc3)c3ccc4ccc5c6ccccc6ccc5c4c3)cc2)cc1 ; c1ccc2cc(-c3ccc(N(c4ccc5ccccc5c4)c4ccc5ccc6c7ccccc7ccc6c5c4)cc3)ccc2c1 ; c1ccc(-c2ccc(N(c3ccc(-c4ccccc4)cc3)c3ccc4c(ccc5c6ccccc6ccc45)c3)cc2)cc1]

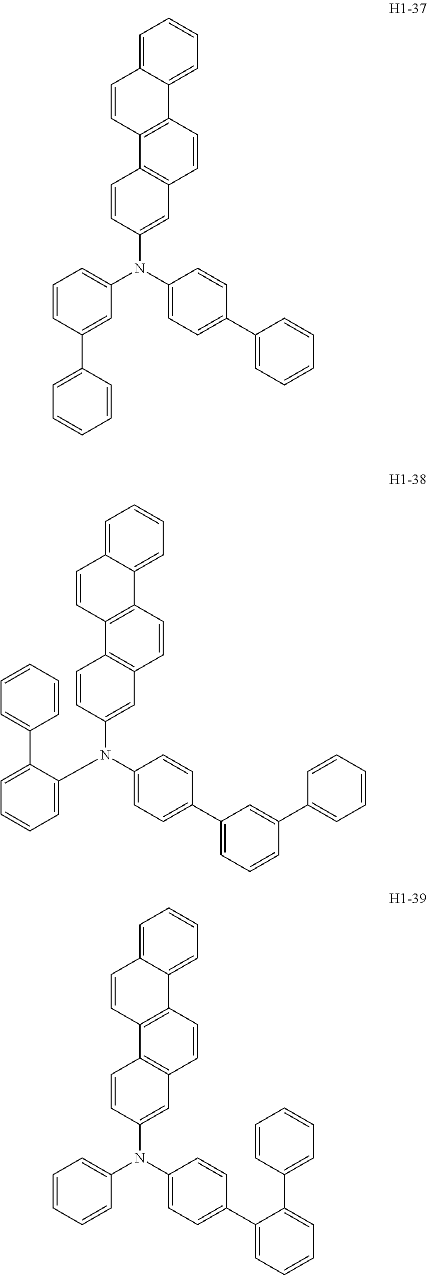 [SMILES: c1ccc(-c2ccc(N(c3cccc(-c4ccccc4)c3)c3ccc4c(ccc5c6ccccc6ccc45)c3)cc2)cc1 ; c1ccc(-c2cccc(-c3ccc(N(c4ccc5c(ccc6c7ccccc7ccc56)c4)c4ccccc4-c4ccccc4)cc3)c2)cc1 ; c1ccc(-c2ccccc2-c2ccc(N(c3ccccc3)c3ccc4c(ccc5c6ccccc6ccc45)c3)cc2)cc1]

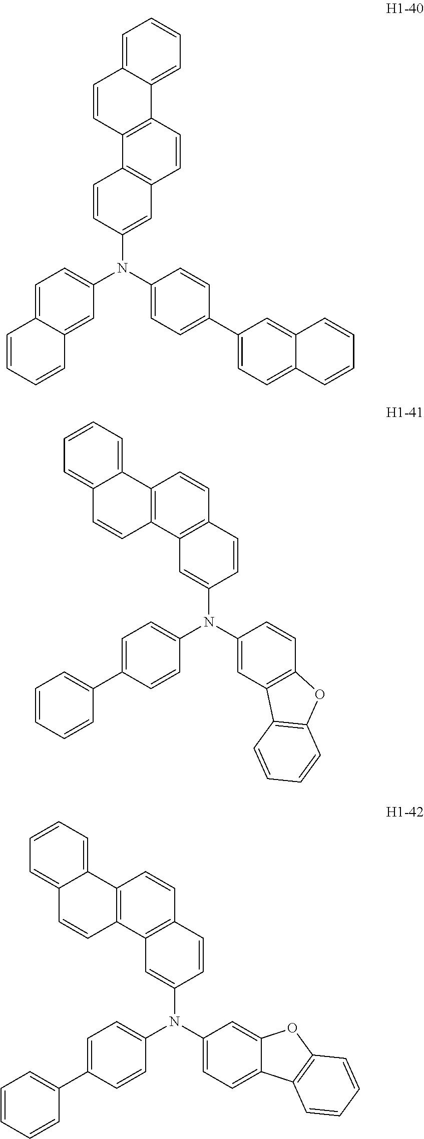 [SMILES: c1ccc2cc(-c3ccc(N(c4ccc5ccccc5c4)c4ccc5c(ccc6c7ccccc7ccc56)c4)cc3)ccc2c1 ; c1ccc(-c2ccc(N(c3ccc4ccc5c6ccccc6ccc5c4c3)c3ccc4oc5ccccc5c4c3)cc2)cc1 ; c1ccc(-c2ccc(N(c3ccc4c(c3)oc3ccccc34)c3ccc4ccc5c6ccccc6ccc5c4c3)cc2)cc1]

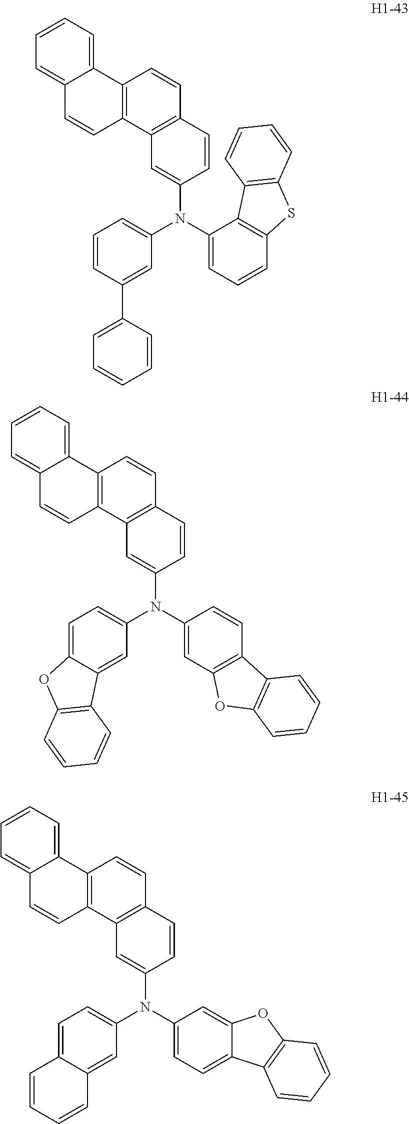 [SMILES: c1ccc(-c2cccc(N(c3ccc4ccc5c6ccccc6ccc5c4c3)c3cccc4sc5ccccc5c34)c2)cc1 ; c1ccc2c(c1)ccc1c3cc(N(c4ccc5c(c4)oc4ccccc45)c4ccc5oc6ccccc6c5c4)ccc3ccc21 ; c1ccc2cc(N(c3ccc4c(c3)oc3ccccc34)c3ccc4ccc5c6ccccc6ccc5c4c3)ccc2c1]

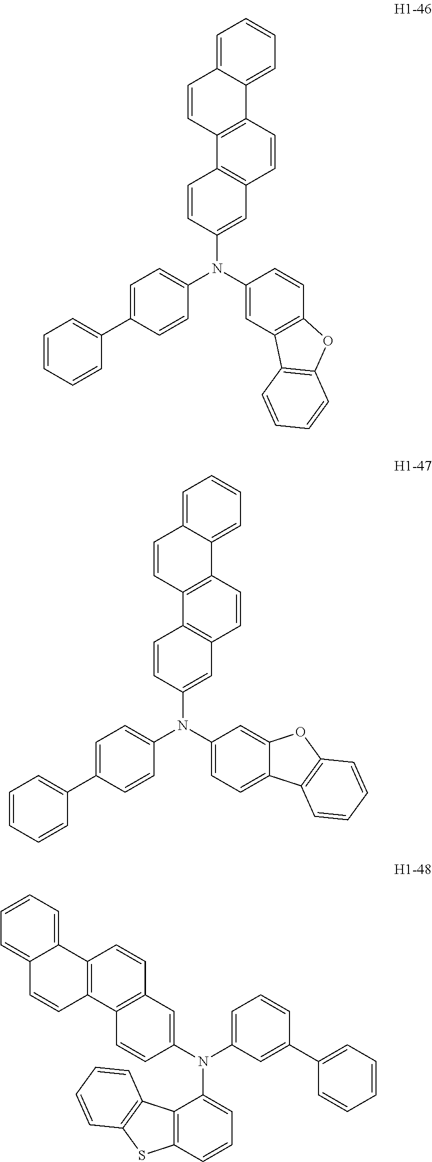 [SMILES: c1ccc(-c2ccc(N(c3ccc4c(ccc5c6ccccc6ccc45)c3)c3ccc4oc5ccccc5c4c3)cc2)cc1 ; c1ccc(-c2ccc(N(c3ccc4c(ccc5c6ccccc6ccc45)c3)c3ccc4c(c3)oc3ccccc34)cc2)cc1 ; c1ccc(-c2cccc(N(c3ccc4c(ccc5c6ccccc6ccc45)c3)c3cccc4sc5ccccc5c34)c2)cc1]

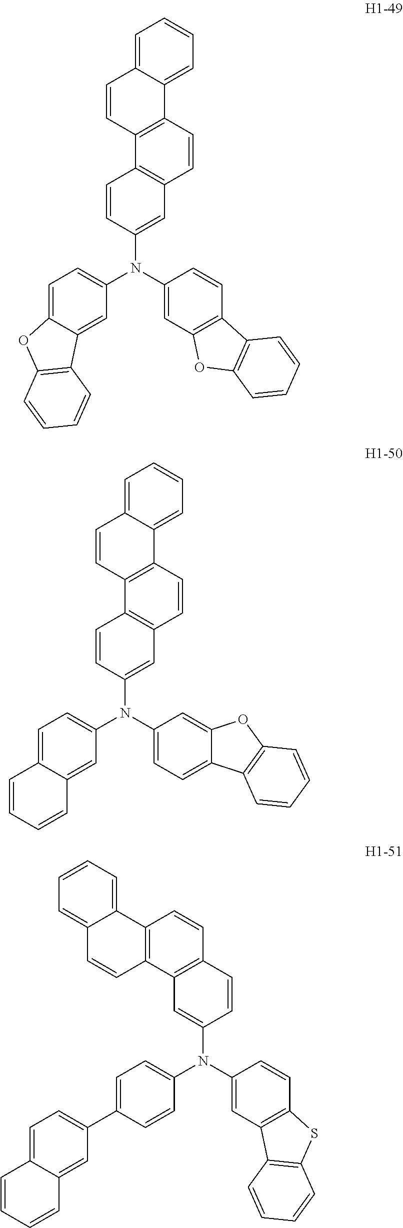 [SMILES: c1ccc2c(c1)ccc1c3ccc(N(c4ccc5c(c4)oc4ccccc45)c4ccc5oc6ccccc6c5c4)cc3ccc21 ; c1ccc2cc(N(c3ccc4c(ccc5c6ccccc6ccc45)c3)c3ccc4c(c3)oc3ccccc34)ccc2c1 ; c1ccc2cc(-c3ccc(N(c4ccc5ccc6c7ccccc7ccc6c5c4)c4ccc5sc6ccccc6c5c4)cc3)ccc2c1]

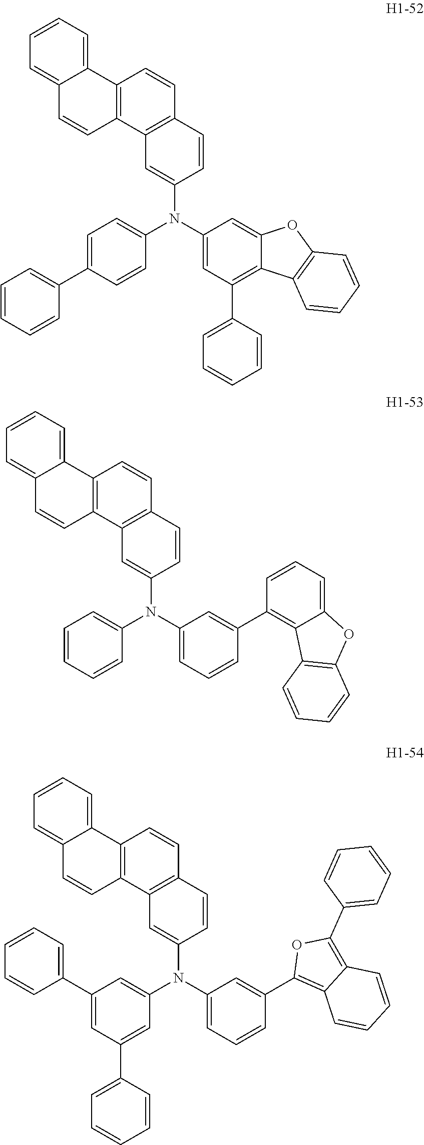 [SMILES: c1ccc(-c2ccc(N(c3cc(-c4ccccc4)c4c(c3)oc3ccccc34)c3ccc4ccc5c6ccccc6ccc5c4c3)cc2)cc1 ; c1ccc(N(c2cccc(-c3cccc4oc5ccccc5c34)c2)c2ccc3ccc4c5ccccc5ccc4c3c2)cc1 ; c1ccc(-c2cc(-c3ccccc3)cc(N(c3cccc(-c4oc(-c5ccccc5)c5ccccc45)c3)c3ccc4ccc5c6ccccc6ccc5c4c3)c2)cc1]

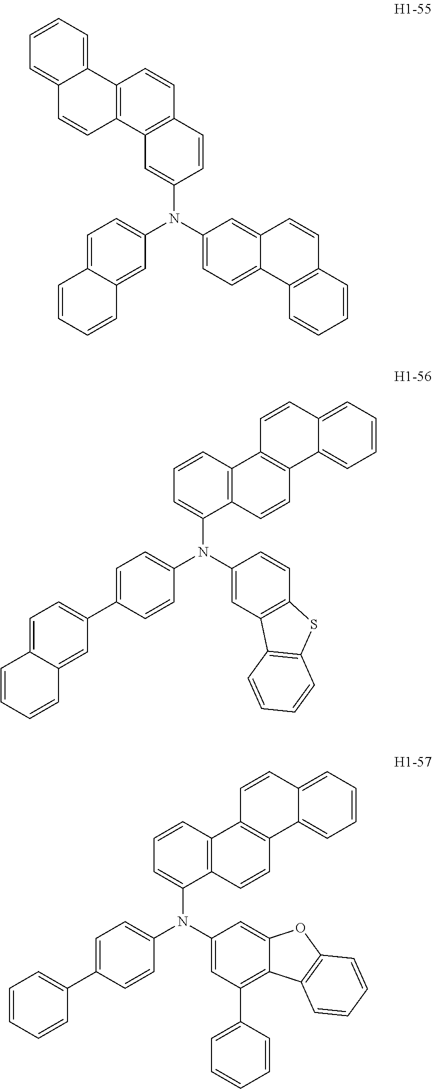 [SMILES: c1ccc2cc(N(c3ccc4c(ccc5ccccc54)c3)c3ccc4ccc5c6ccccc6ccc5c4c3)ccc2c1 ; c1ccc2cc(-c3ccc(N(c4ccc5sc6ccccc6c5c4)c4cccc5c4ccc4c6ccccc6ccc54)cc3)ccc2c1 ; c1ccc(-c2ccc(N(c3cc(-c4ccccc4)c4c(c3)oc3ccccc34)c3cccc4c3ccc3c5ccccc5ccc43)cc2)cc1]

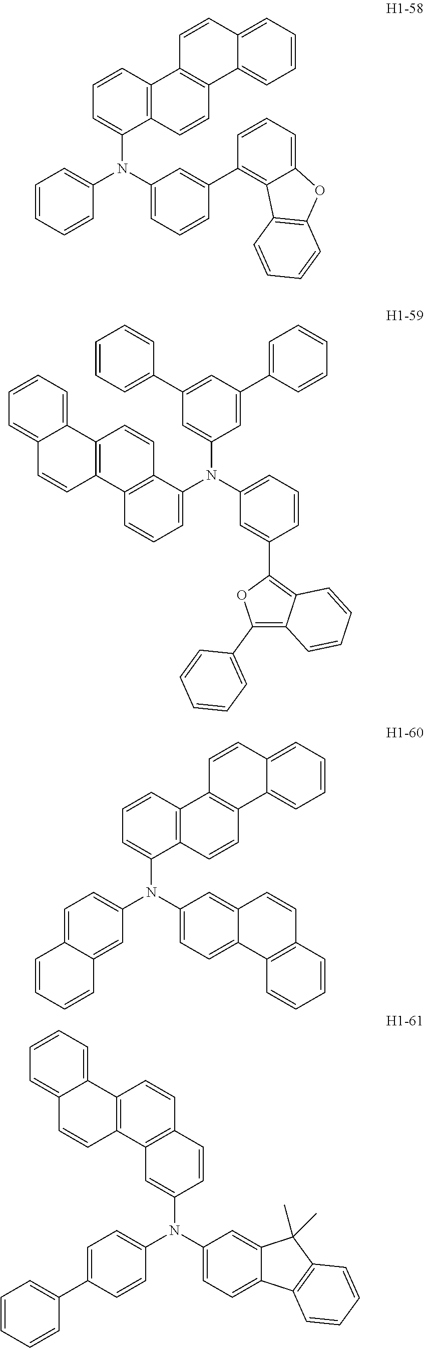

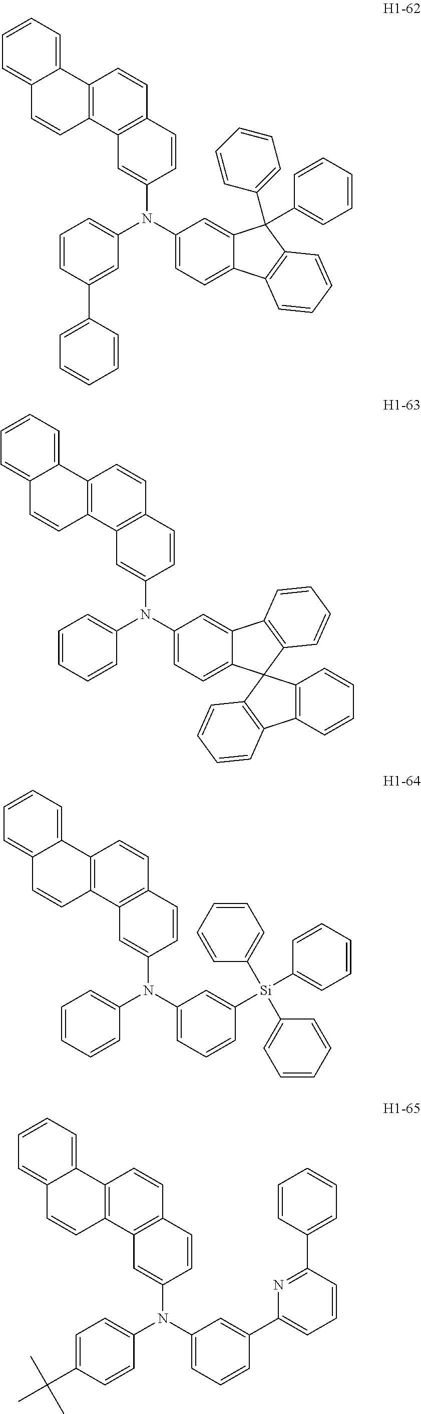 [SMILES: c1ccc(-c2cccc(N(c3ccc4c(c3)C(c3ccccc3)(c3ccccc3)c3ccccc3-4)c3ccc4ccc5c6ccccc6ccc5c4c3)c2)cc1 ; c1ccc(N(c2ccc3c(c2)-c2ccccc2C32c3ccccc3-c3ccccc32)c2ccc3ccc4c5ccccc5ccc4c3c2)cc1 ; c1ccc(N(c2cccc([Si](c3ccccc3)(c3ccccc3)c3ccccc3)c2)c2ccc3ccc4c5ccccc5ccc4c3c2)cc1 ; CC(C)(C)c1ccc(N(c2cccc(-c3cccc(-c4ccccc4)n3)c2)c2ccc3ccc4c5ccccc5ccc4c3c2)cc1]

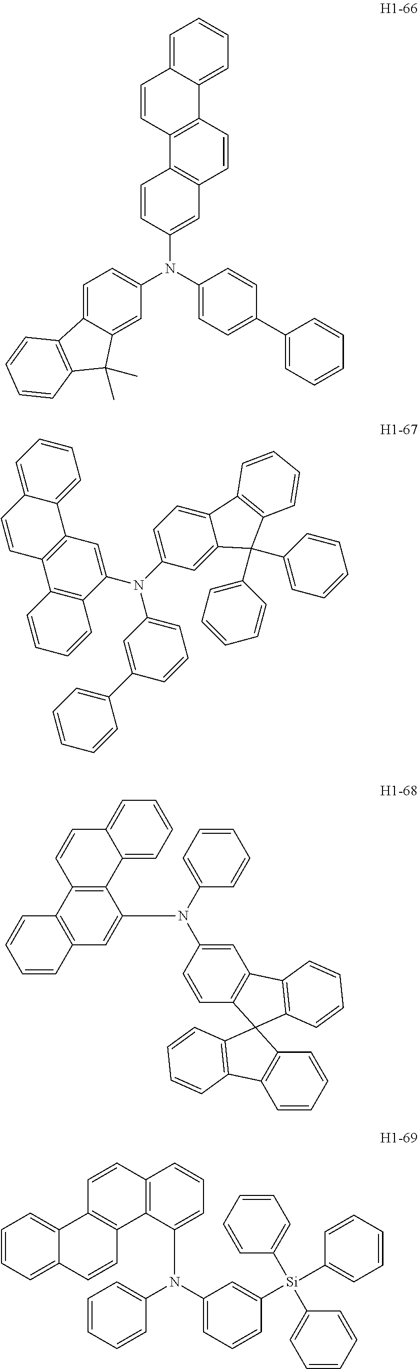

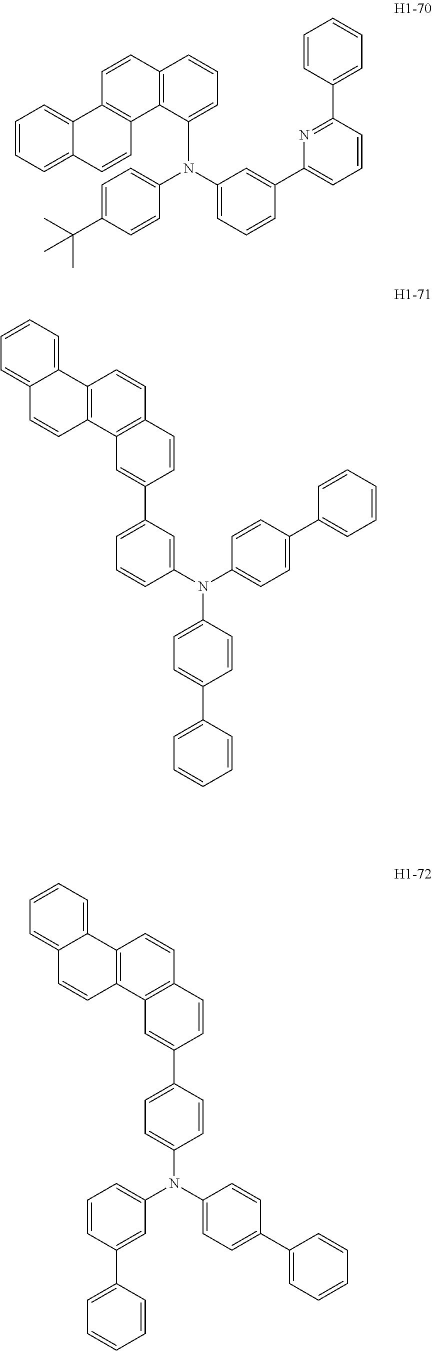 [SMILES: CC(C)(C)c1ccc(N(c2cccc(-c3cccc(-c4ccccc4)n3)c2)c2cccc3ccc4c5ccccc5ccc4c23)cc1 ; c1ccc(-c2ccc(N(c3ccc(-c4ccccc4)cc3)c3cccc(-c4ccc5ccc6c7ccccc7ccc6c5c4)c3)cc2)cc1 ; c1ccc(-c2ccc(N(c3ccc(-c4ccc5ccc6c7ccccc7ccc6c5c4)cc3)c3cccc(-c4ccccc4)c3)cc2)cc1]

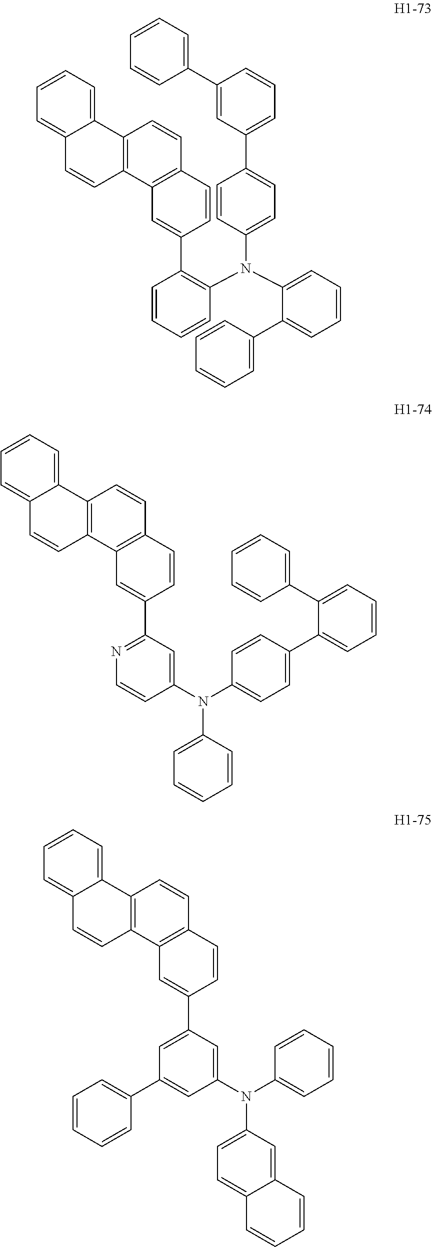 [SMILES: c1ccc(-c2cccc(-c3ccc(N(c4ccccc4-c4ccccc4)c4ccccc4-c4ccc5ccc6c7ccccc7ccc6c5c4)cc3)c2)cc1 ; c1ccc(-c2ccccc2-c2ccc(N(c3ccccc3)c3ccnc(-c4ccc5ccc6c7ccccc7ccc6c5c4)c3)cc2)cc1 ; c1ccc(-c2cc(-c3ccc4ccc5c6ccccc6ccc5c4c3)cc(N(c3ccccc3)c3ccc4ccccc4c3)c2)cc1]

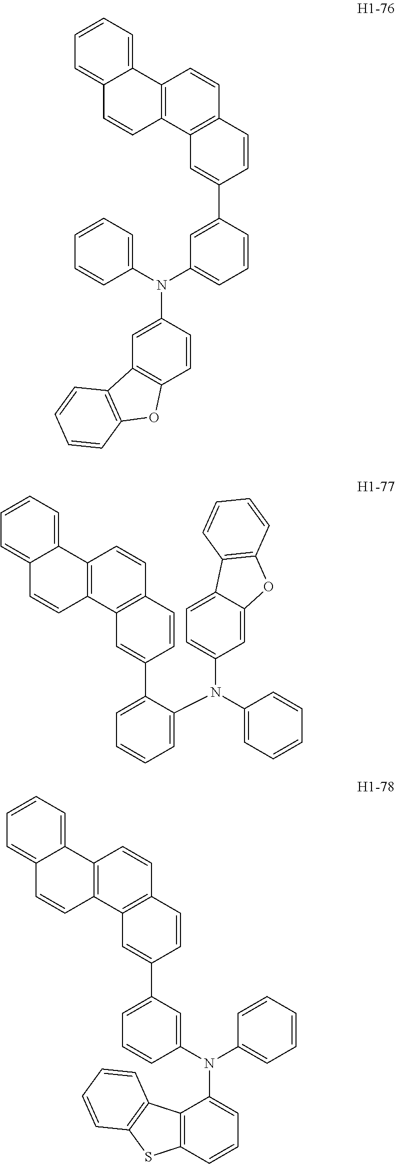 [SMILES: c1ccc(N(c2cccc(-c3ccc4ccc5c6ccccc6ccc5c4c3)c2)c2ccc3oc4ccccc4c3c2)cc1 ; c1ccc(N(c2ccc3c(c2)oc2ccccc23)c2ccccc2-c2ccc3ccc4c5ccccc5ccc4c3c2)cc1 ; c1ccc(N(c2cccc(-c3ccc4ccc5c6ccccc6ccc5c4c3)c2)c2cccc3sc4ccccc4c23)cc1]

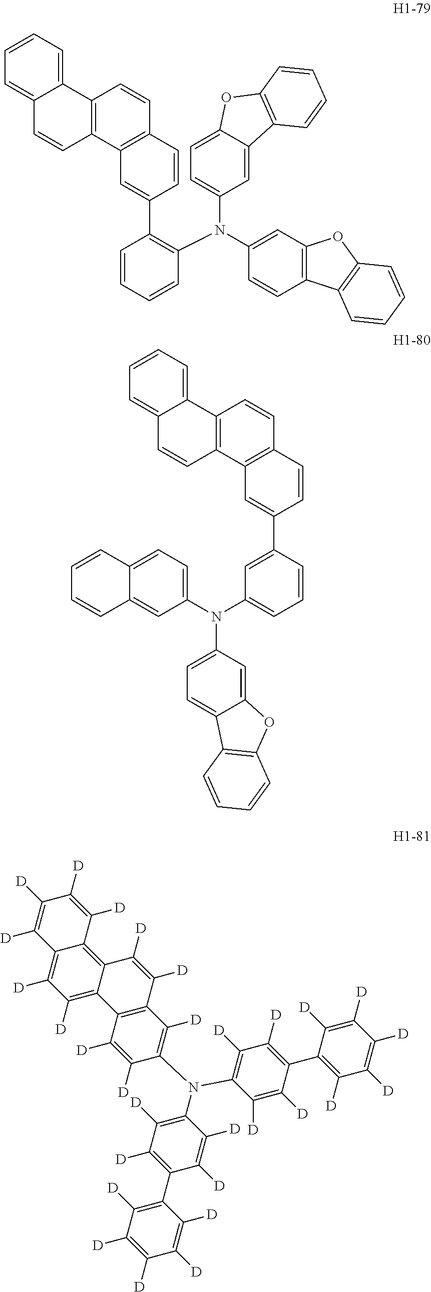 [SMILES: c1ccc(N(c2ccc3c(c2)oc2ccccc23)c2ccc3oc4ccccc4c3c2)c(-c2ccc3ccc4c5ccccc5ccc4c3c2)c1 ; c1cc(-c2ccc3ccc4c5ccccc5ccc4c3c2)cc(N(c2ccc3ccccc3c2)c2ccc3c(c2)oc2ccccc23)c1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c5c([2H])c([2H])c([2H])c([2H])c5c([2H])c([2H])c43)c([2H])c2[2H])c([2H])c1[2H]]

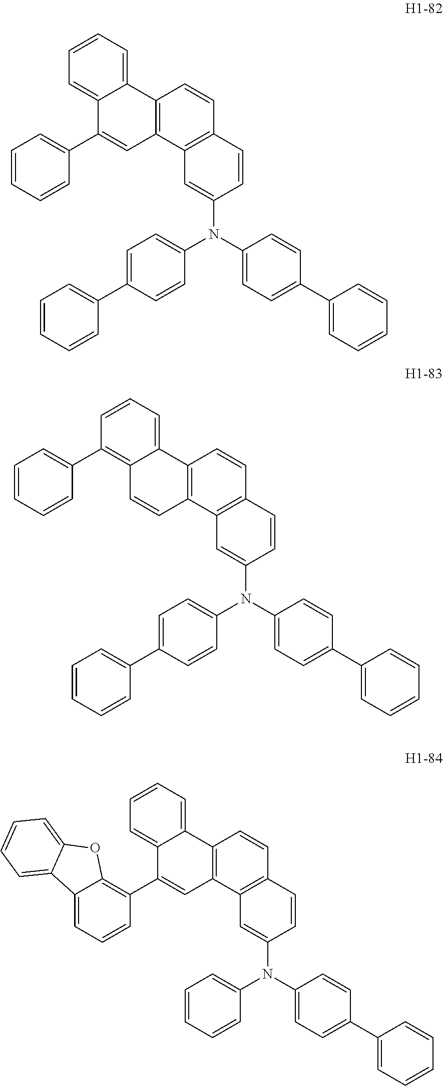 [SMILES: c1ccc(-c2ccc(N(c3ccc(-c4ccccc4)cc3)c3ccc4ccc5c6ccccc6c(-c6ccccc6)cc5c4c3)cc2)cc1 ; c1ccc(-c2ccc(N(c3ccc(-c4ccccc4)cc3)c3ccc4ccc5c6cccc(-c7ccccc7)c6ccc5c4c3)cc2)cc1 ; c1ccc(-c2ccc(N(c3ccccc3)c3ccc4ccc5c6ccccc6c(-c6cccc7c6oc6ccccc67)cc5c4c3)cc2)cc1]

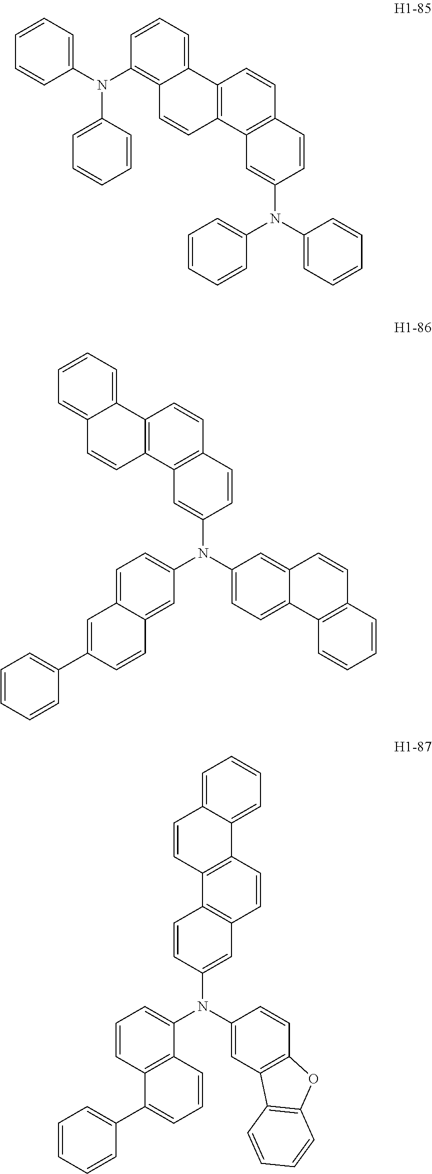 [SMILES: c1ccc(N(c2ccccc2)c2ccc3ccc4c5cccc(N(c6ccccc6)c6ccccc6)c5ccc4c3c2)cc1 ; c1ccc(-c2ccc3cc(N(c4ccc5c(ccc6ccccc65)c4)c4ccc5ccc6c7ccccc7ccc6c5c4)ccc3c2)cc1 ; c1ccc(-c2cccc3c(N(c4ccc5c(ccc6c7ccccc7ccc56)c4)c4ccc5oc6ccccc6c5c4)cccc23)cc1]

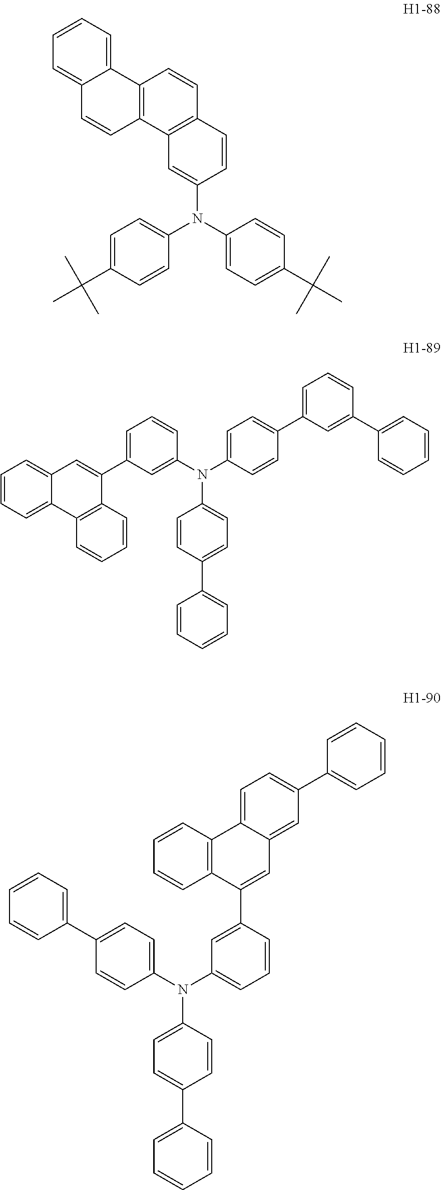 [SMILES: CC(C)(C)c1ccc(N(c2ccc(C(C)(C)C)cc2)c2ccc3ccc4c5ccccc5ccc4c3c2)cc1 ; c1ccc(-c2ccc(N(c3ccc(-c4cccc(-c5ccccc5)c4)cc3)c3cccc(-c4cc5ccccc5c5ccccc45)c3)cc2)cc1 ; c1ccc(-c2ccc(N(c3ccc(-c4ccccc4)cc3)c3cccc(-c4cc5cc(-c6ccccc6)ccc5c5ccccc45)c3)cc2)cc1]

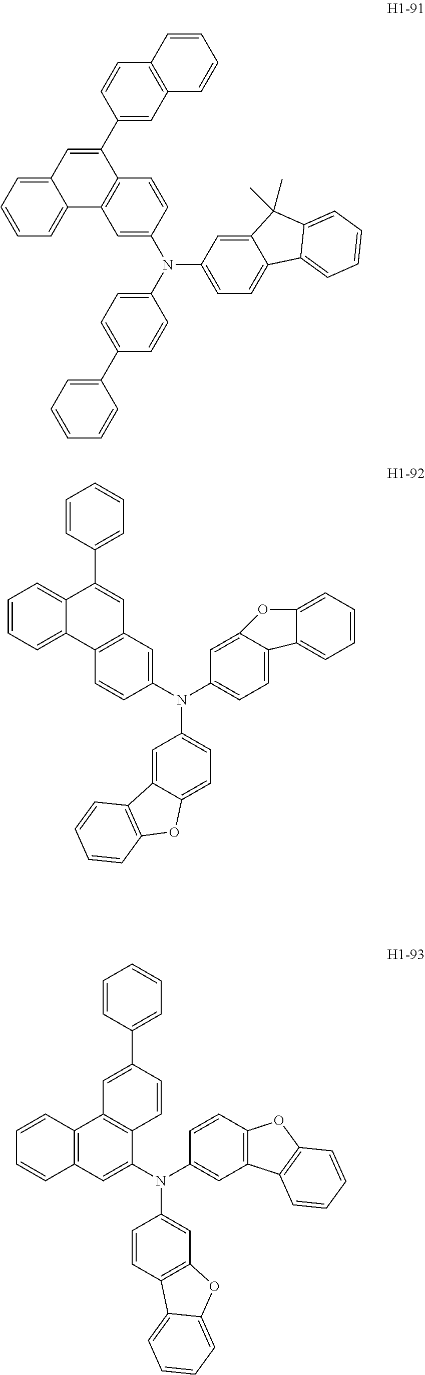

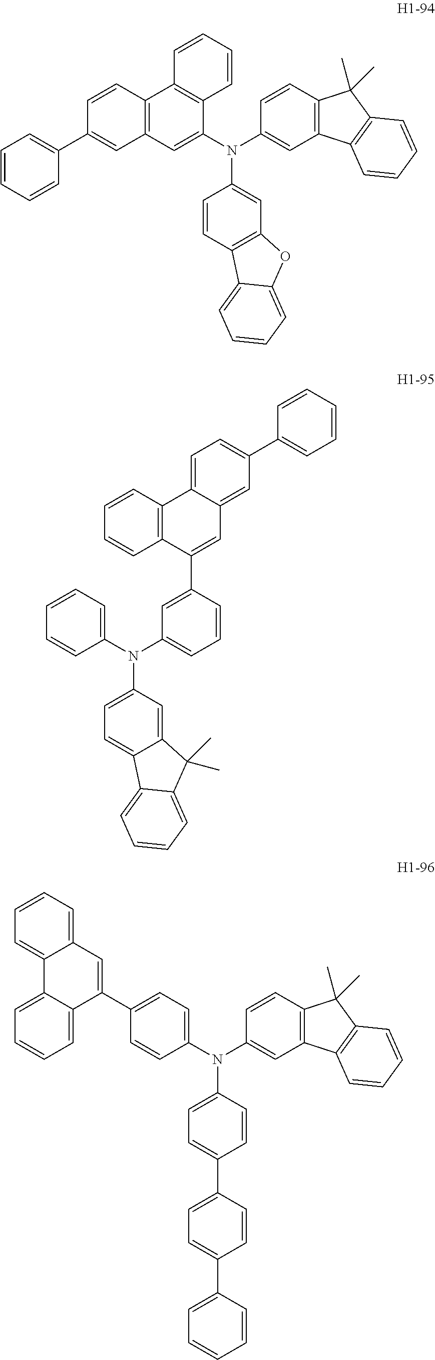 [SMILES: CC1(C)c2ccccc2-c2cc(N(c3ccc4c(c3)oc3ccccc34)c3cc4cc(-c5ccccc5)ccc4c4ccccc34)ccc21 ; CC1(C)c2ccccc2-c2ccc(N(c3ccccc3)c3cccc(-c4cc5cc(-c6ccccc6)ccc5c5ccccc45)c3)cc21 ; CC1(C)c2ccccc2-c2cc(N(c3ccc(-c4ccc(-c5ccccc5)cc4)cc3)c3ccc(-c4cc5ccccc5c5ccccc45)cc3)ccc21]

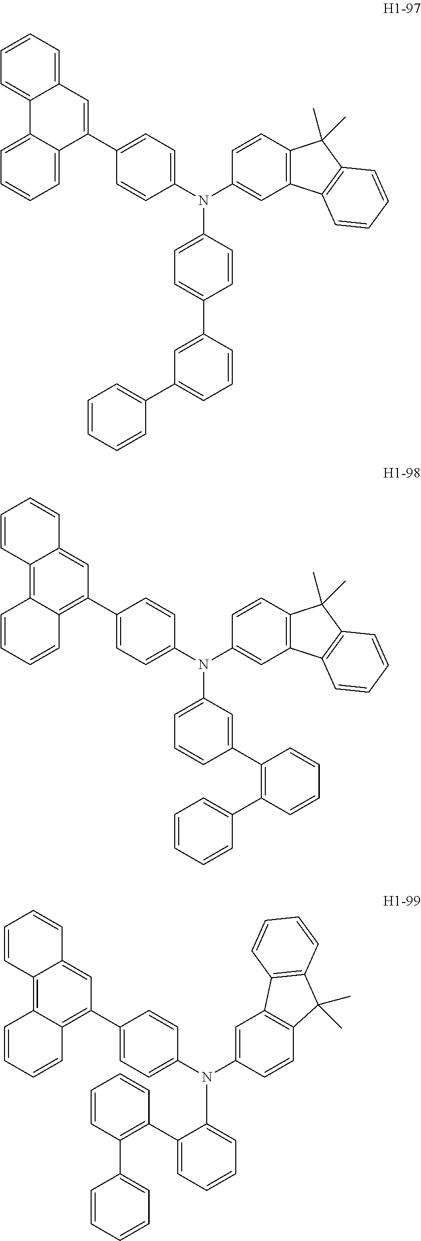 [SMILES: CC1(C)c2ccccc2-c2cc(N(c3ccc(-c4cccc(-c5ccccc5)c4)cc3)c3ccc(-c4cc5ccccc5c5ccccc45)cc3)ccc21 ; CC1(C)c2ccccc2-c2cc(N(c3ccc(-c4cc5ccccc5c5ccccc45)cc3)c3cccc(-c4ccccc4-c4ccccc4)c3)ccc21 ; CC1(C)c2ccccc2-c2cc(N(c3ccc(-c4cc5ccccc5c5ccccc45)cc3)c3ccccc3-c3ccccc3-c3ccccc3)ccc21]

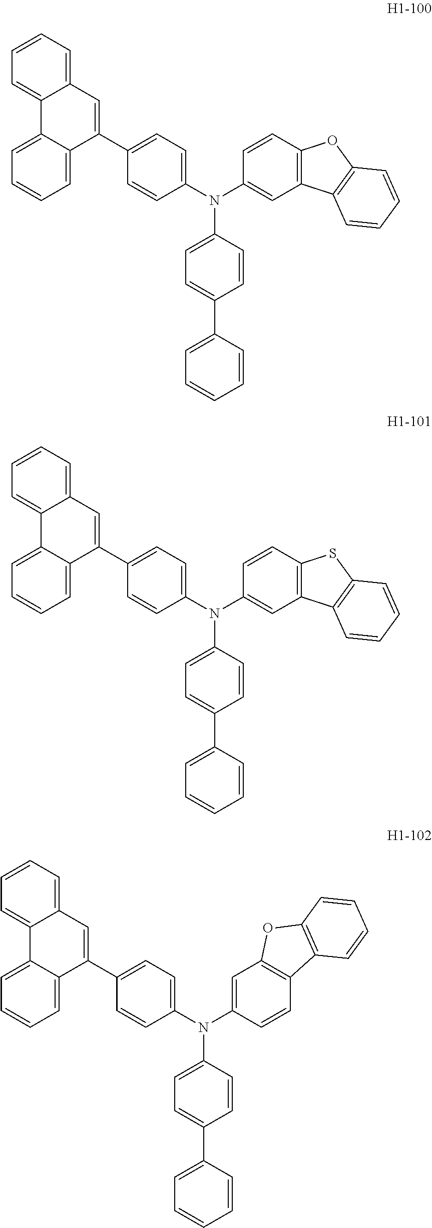 [SMILES: c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4sc5ccccc5c4c3)cc2)cc1 ; c1ccc(-c2ccc(N(c3ccc(-c4cc5ccccc5c5ccccc45)cc3)c3ccc4c(c3)oc3ccccc34)cc2)cc1]

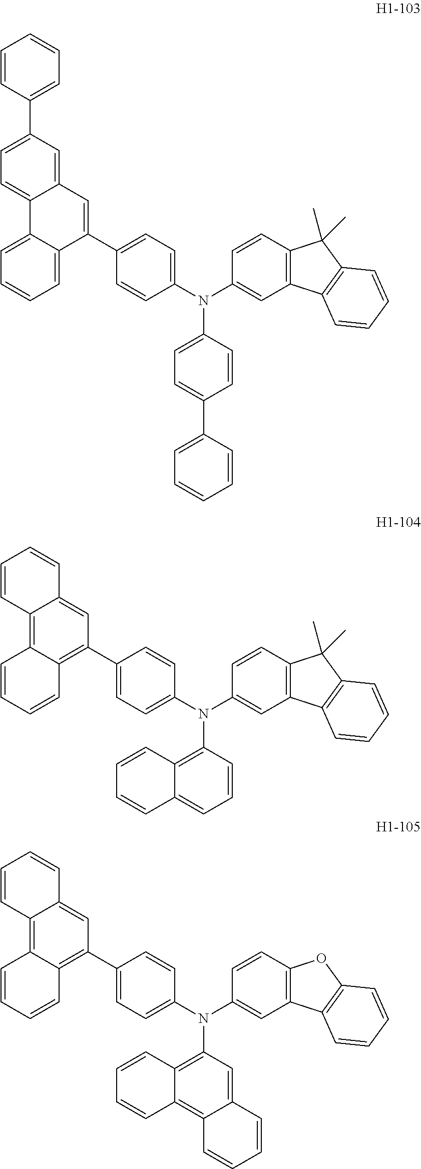 [SMILES: CC1(C)c2ccccc2-c2cc(N(c3ccc(-c4ccccc4)cc3)c3ccc(-c4cc5cc(-c6ccccc6)ccc5c5ccccc45)cc3)ccc21 ; CC1(C)c2ccccc2-c2cc(N(c3ccc(-c4cc5ccccc5c5ccccc45)cc3)c3cccc4ccccc34)ccc21 ; c1ccc2c(c1)cc(-c1ccc(N(c3ccc4oc5ccccc5c4c3)c3cc4ccccc4c4ccccc34)cc1)c1ccccc12]

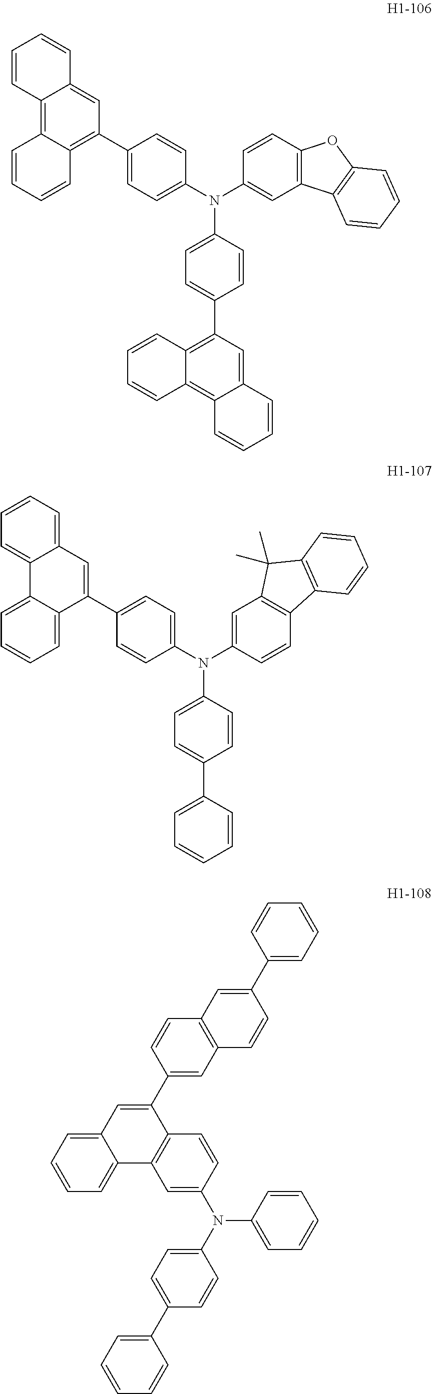 [SMILES: c1ccc2c(c1)cc(-c1ccc(N(c3ccc(-c4cc5ccccc5c5ccccc45)cc3)c3ccc4oc5ccccc5c4c3)cc1)c1ccccc12 ; CC1(C)c2ccccc2-c2ccc(N(c3ccc(-c4ccccc4)cc3)c3ccc(-c4cc5ccccc5c5ccccc45)cc3)cc21 ; c1ccc(-c2ccc(N(c3ccccc3)c3ccc4c(-c5ccc6cc(-c7ccccc7)ccc6c5)cc5ccccc5c4c3)cc2)cc1]

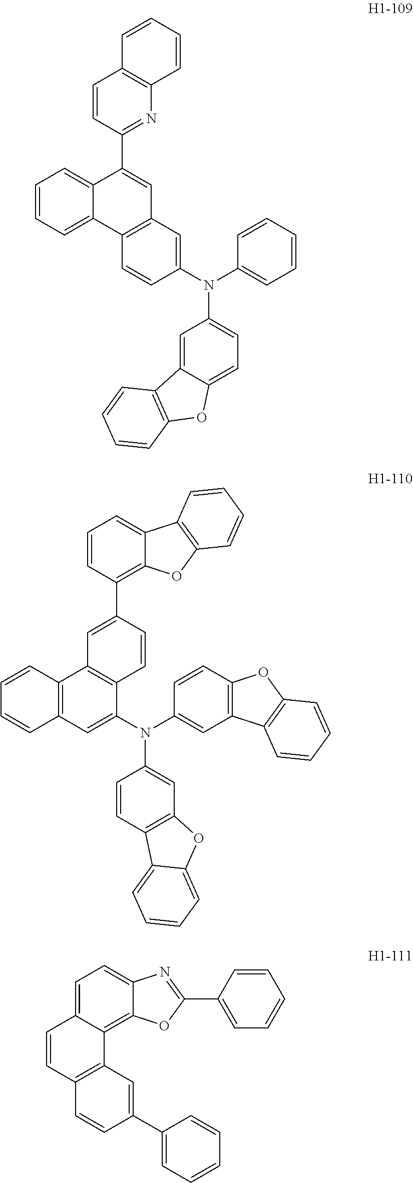 [SMILES: c1ccc(N(c2ccc3c(c2)cc(-c2ccc4ccccc4n2)c2ccccc23)c2ccc3oc4ccccc4c3c2)cc1 ; c1ccc2c(c1)cc(N(c1ccc3c(c1)oc1ccccc13)c1ccc3oc4ccccc4c3c1)c1ccc(-c3cccc4c3oc3ccccc34)cc12 ; c1ccc(-c2ccc3ccc4ccc5nc(-c6ccccc6)oc5c4c3c2)cc1]

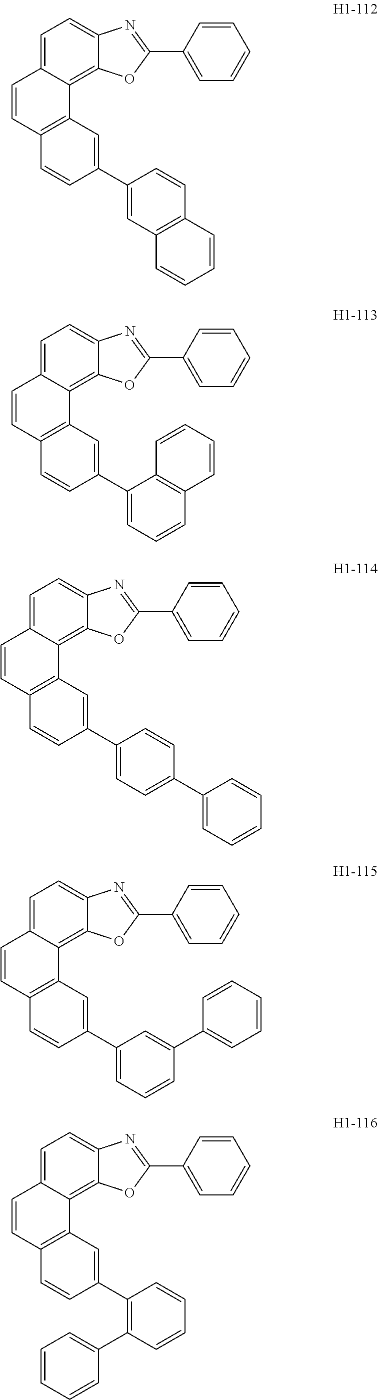

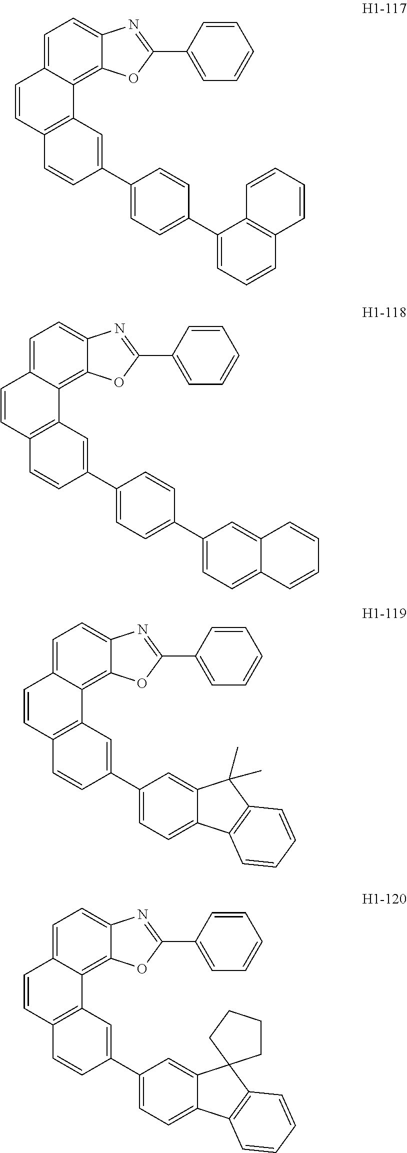

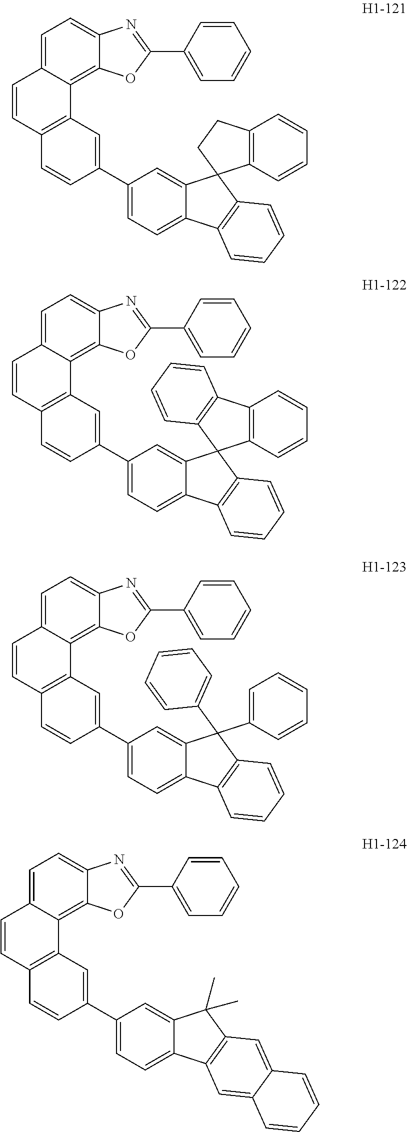

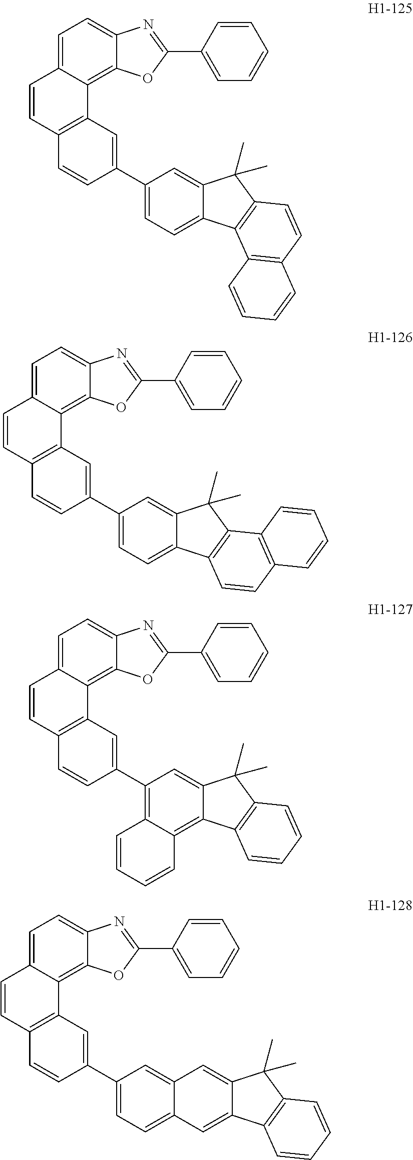

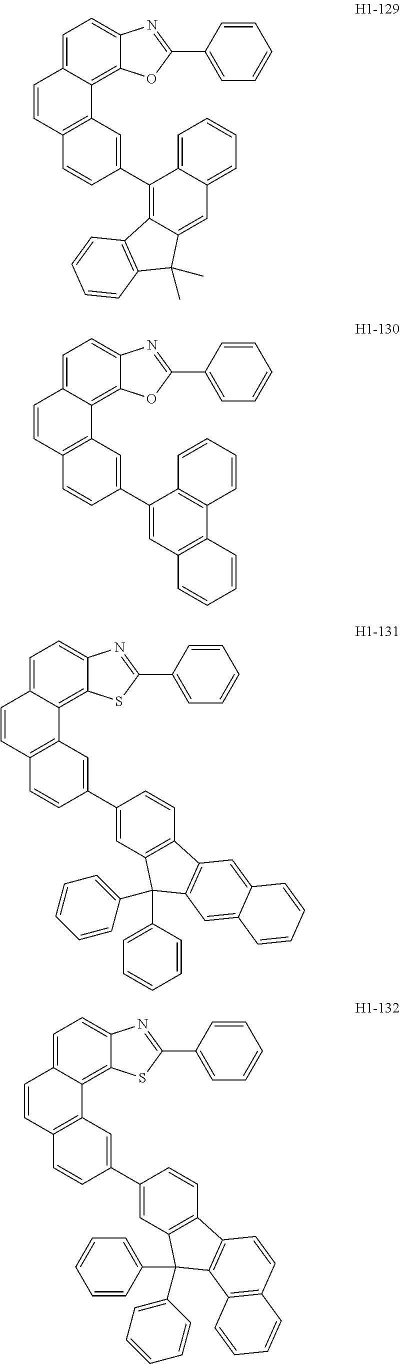

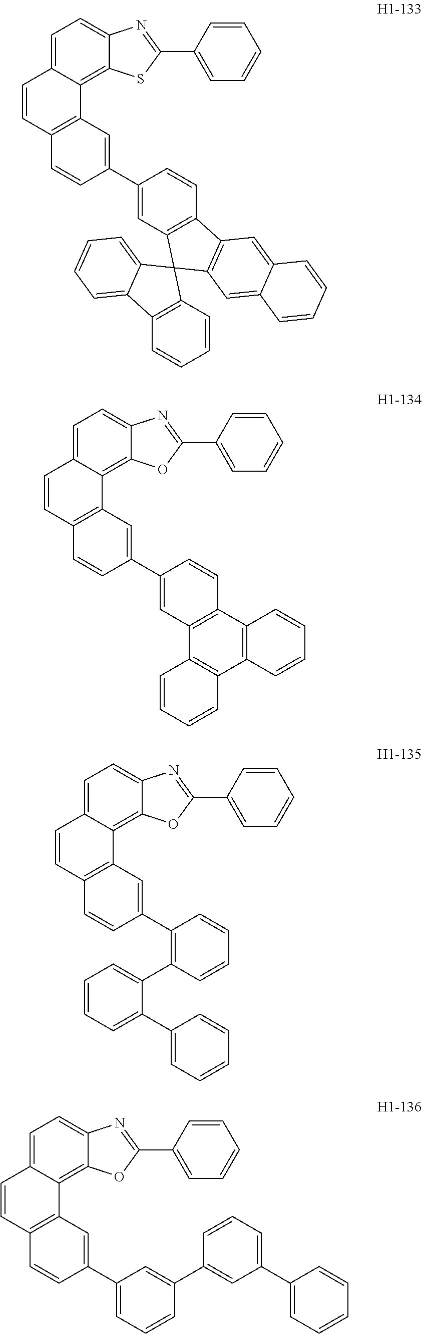

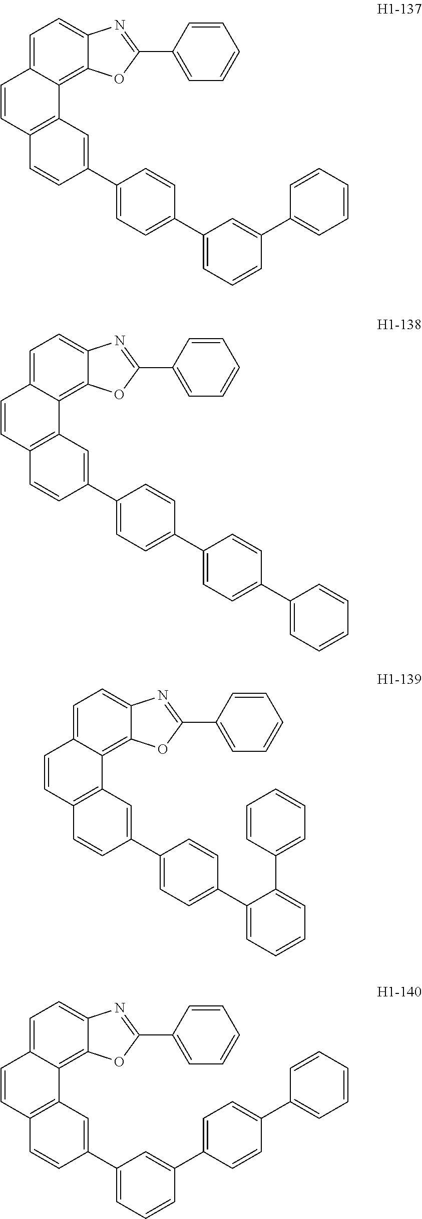 [SMILES: c1ccc(-c2cccc(-c3ccc(-c4ccc5ccc6ccc7nc(-c8ccccc8)oc7c6c5c4)cc3)c2)cc1 ; c1ccc(-c2ccc(-c3ccc(-c4ccc5ccc6ccc7nc(-c8ccccc8)oc7c6c5c4)cc3)cc2)cc1 ; c1ccc(-c2nc3ccc4ccc5ccc(-c6ccc(-c7ccccc7-c7ccccc7)cc6)cc5c4c3o2)cc1 ; c1ccc(-c2ccc(-c3cccc(-c4ccc5ccc6ccc7nc(-c8ccccc8)oc7c6c5c4)c3)cc2)cc1]

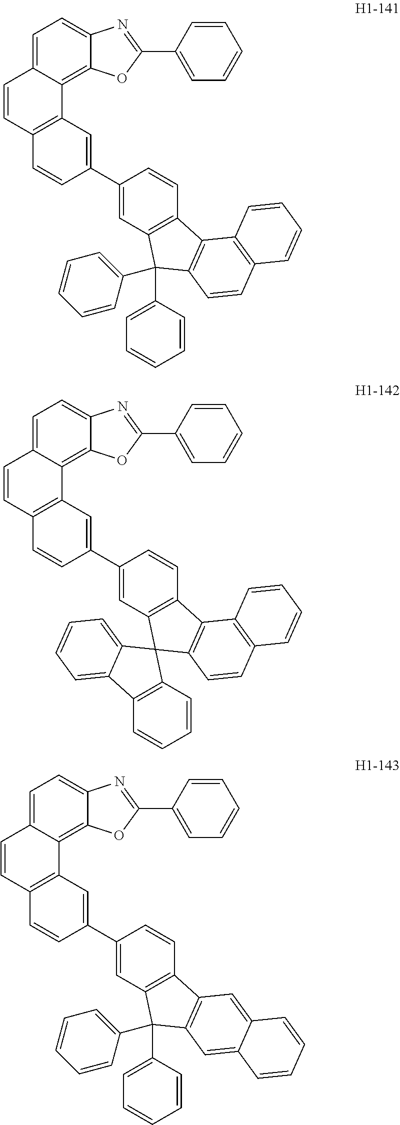

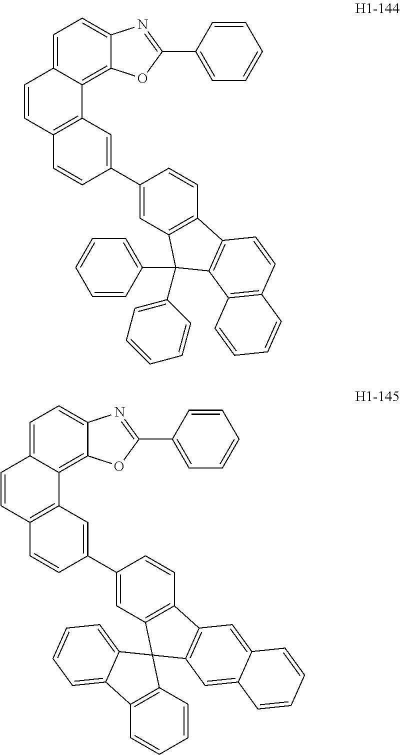 [SMILES: c1ccc(-c2nc3ccc4ccc5ccc(-c6ccc7c(c6)C(c6ccccc6)(c6ccccc6)c6c-7ccc7ccccc67)cc5c4c3o2)cc1 ; c1ccc(-c2nc3ccc4ccc5ccc(-c6ccc7c(c6)C6(c8ccccc8-c8ccccc86)c6cc8ccccc8cc6-7)cc5c4c3o2)cc1]

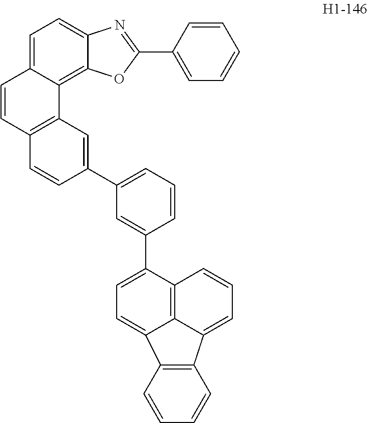 [SMILES: c1ccc(-c2nc3ccc4ccc5ccc(-c6cccc(-c7ccc8c9c(cccc79)-c7ccccc7-8)c6)cc5c4c3o2)cc1]

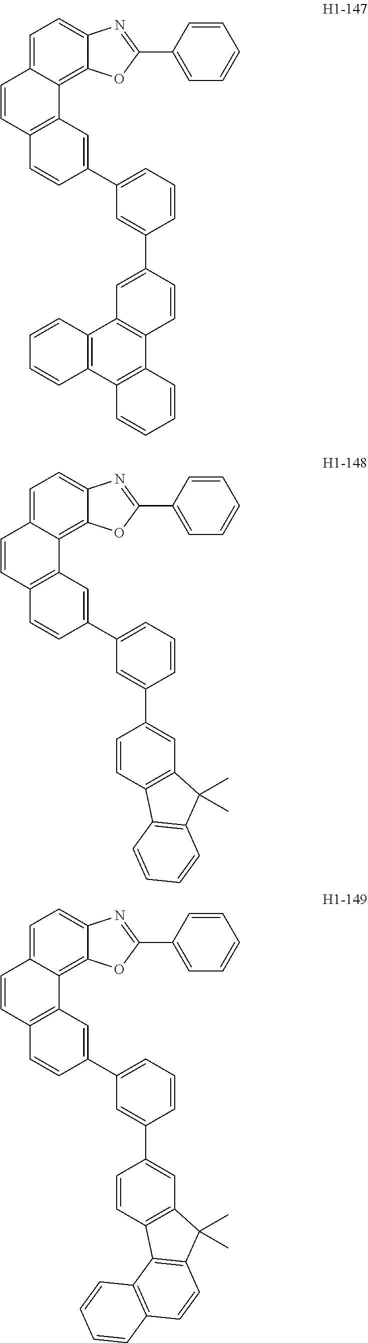

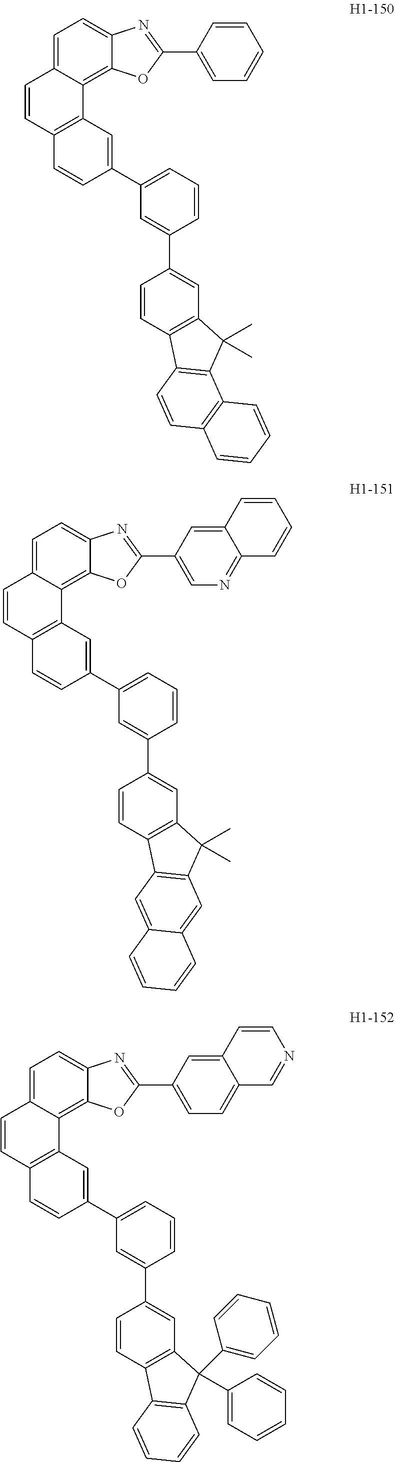

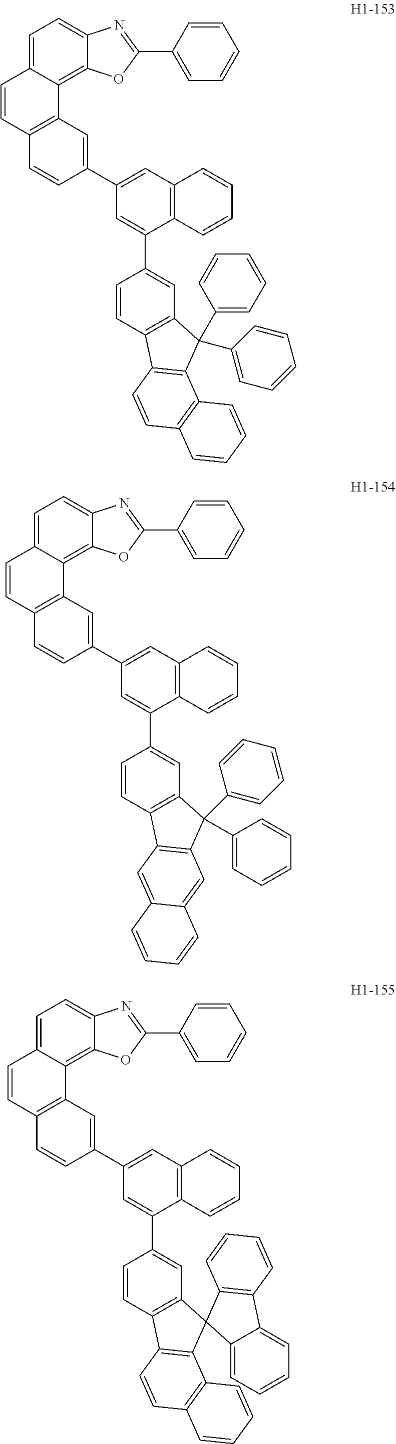

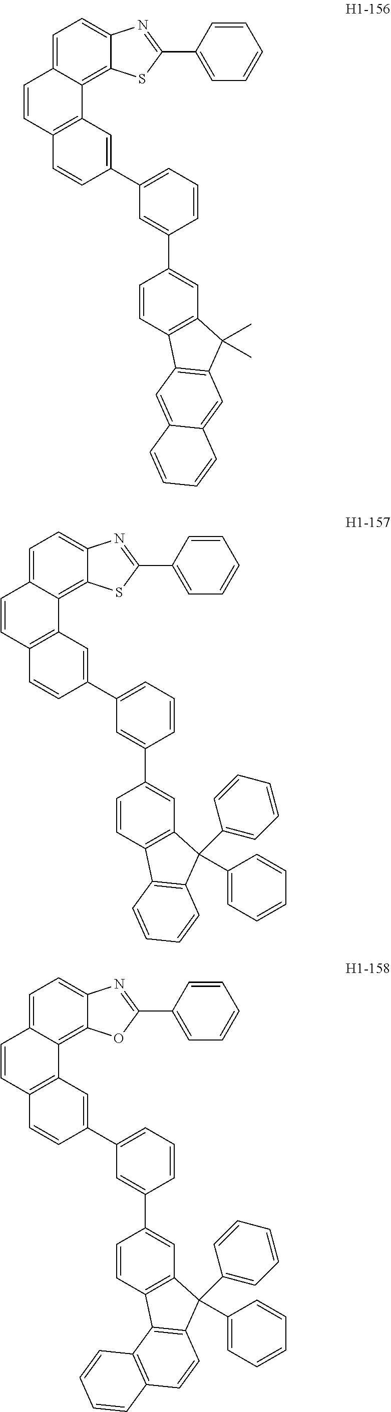

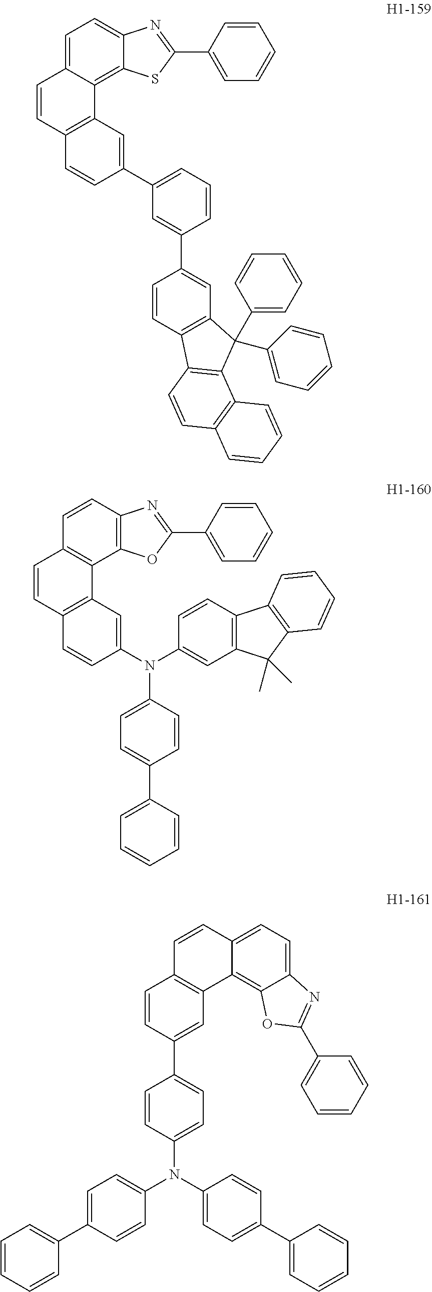

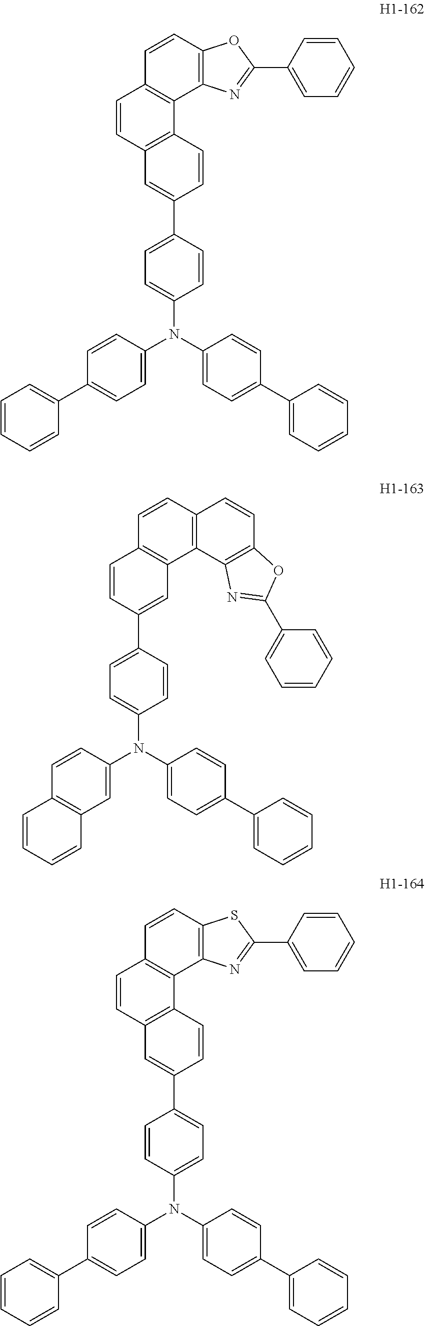 [SMILES: c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1]

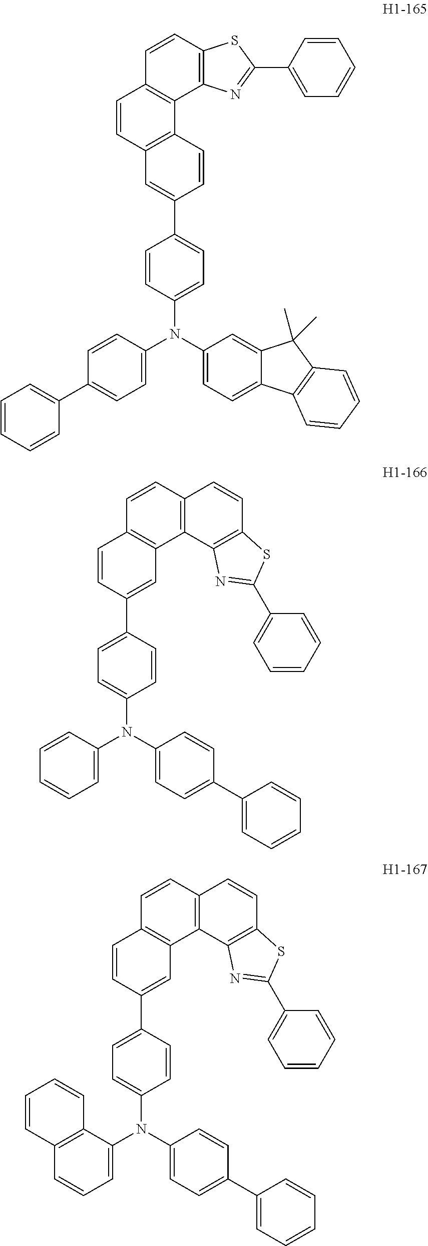 [SMILES: CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1]

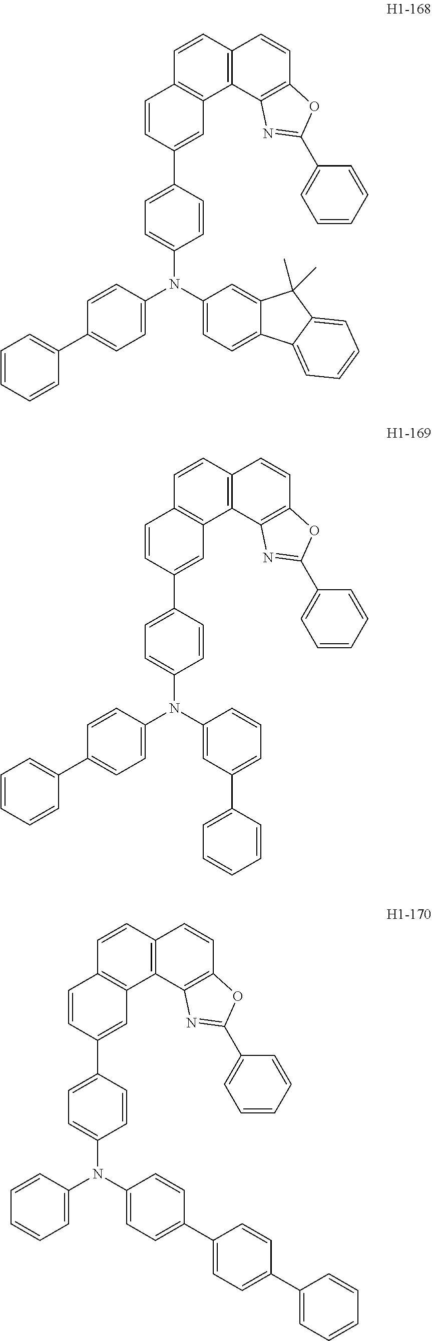 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

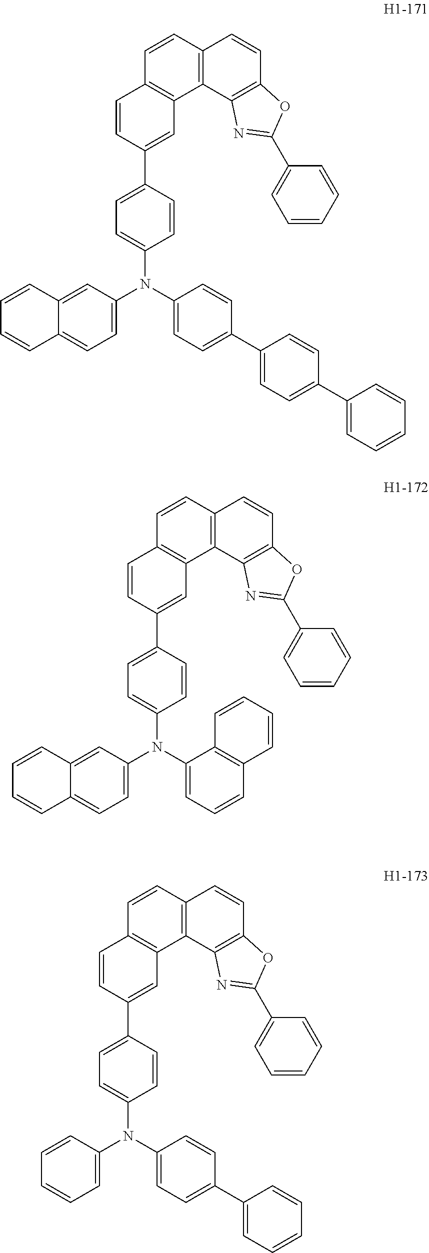 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1]

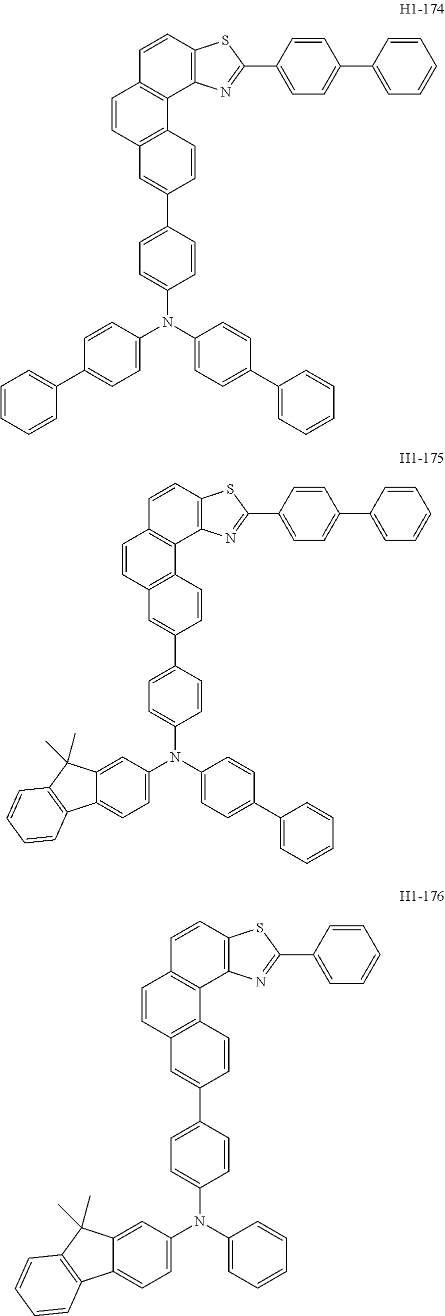 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21]

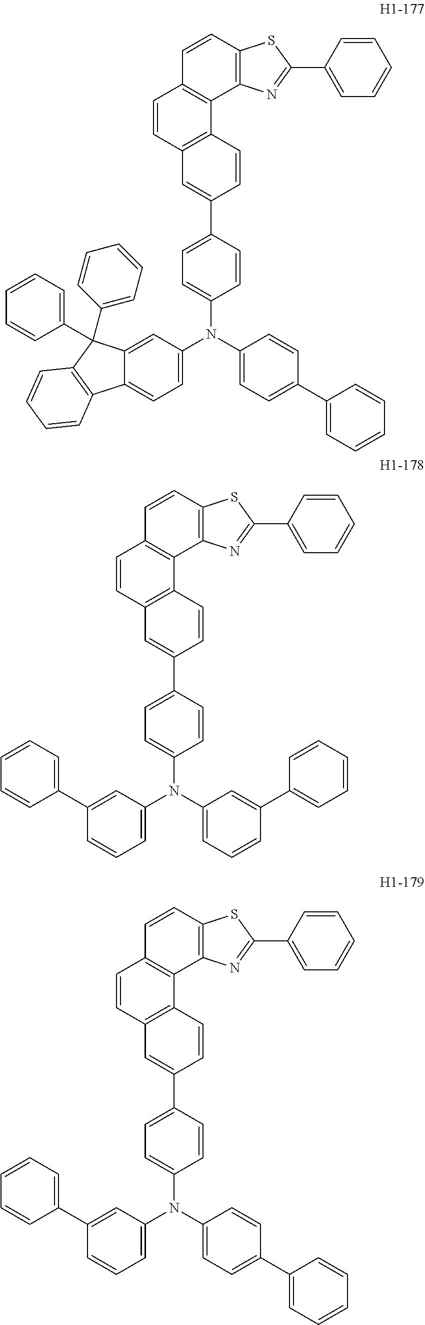 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

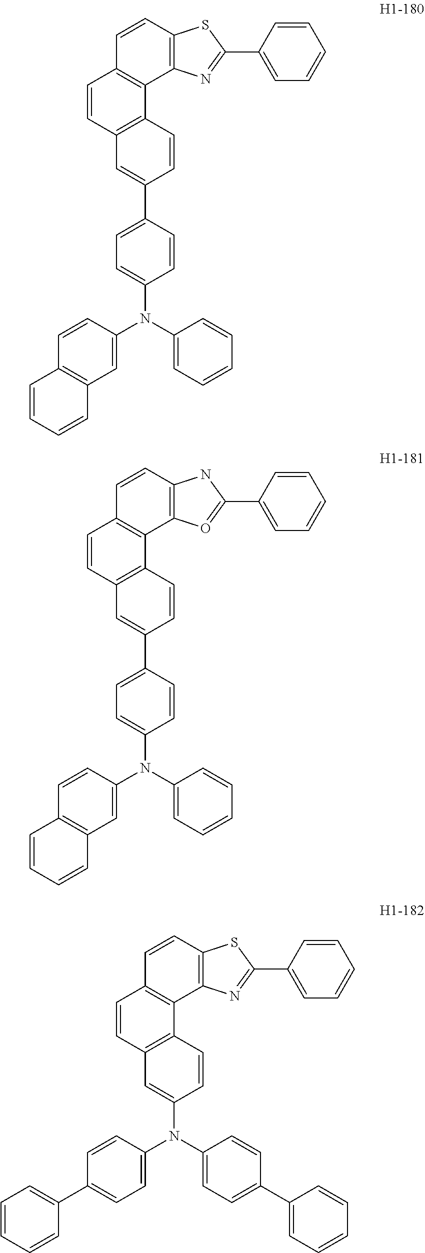

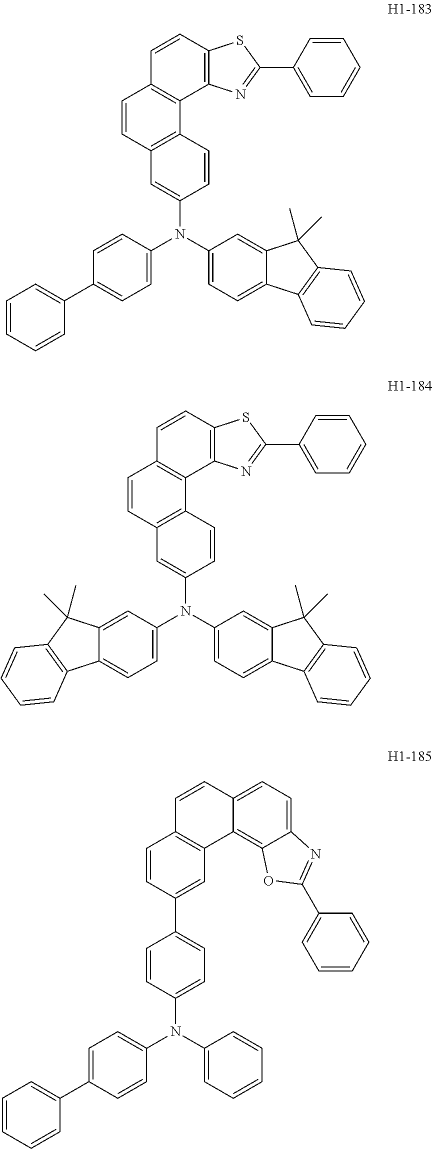 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

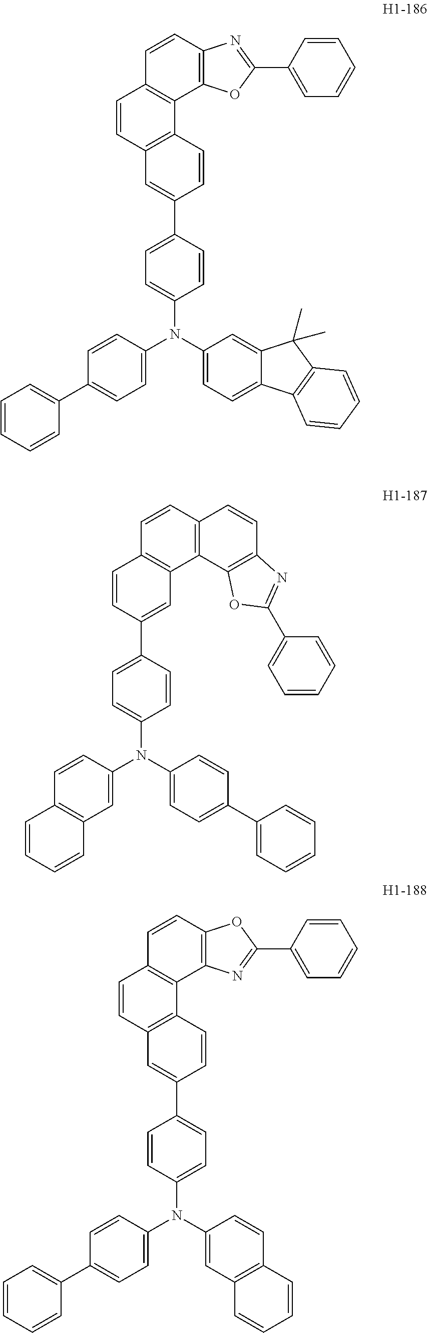 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

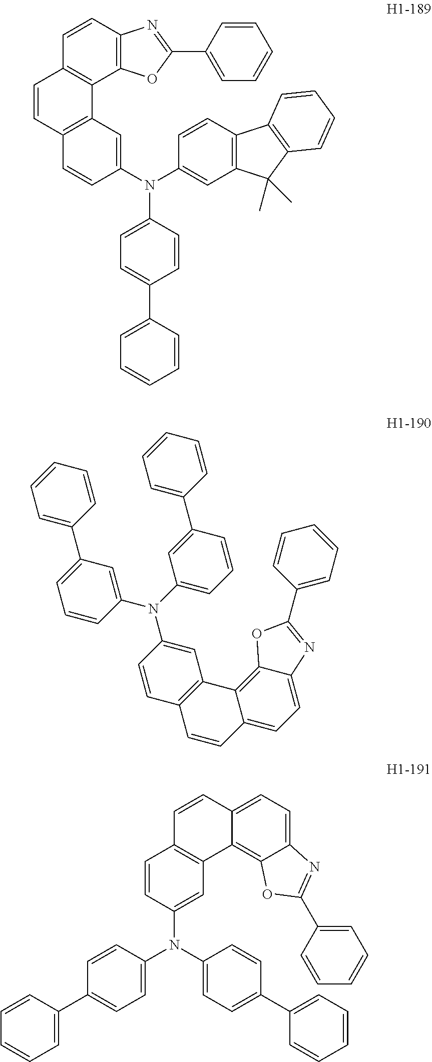 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2cccc(N(c3cccc(-c4ccccc4)c3)c3ccc4ccc5ccc6nc(-c7ccccc7)oc6c5c4c3)c2)cc1 ; c1ccc(-c2ccc(N(c3ccc(-c4ccccc4)cc3)c3ccc4ccc5ccc6nc(-c7ccccc7)oc6c5c4c3)cc2)cc1]

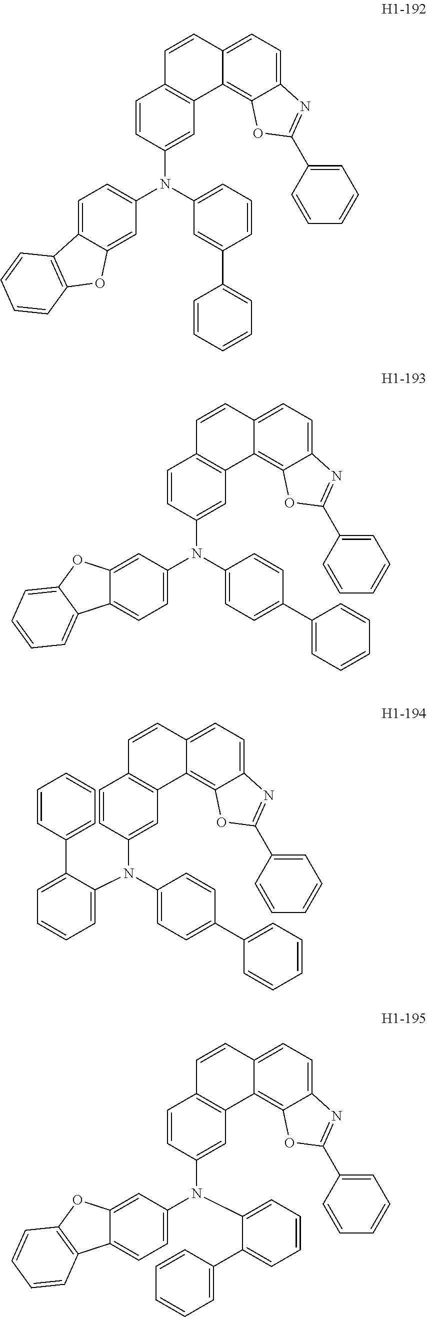 [SMILES: c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3ccccc3-c3ccccc3)cc2)cc1 ; c1ccc(-c2nc3ccc4ccc5ccc(N(c6ccc7c(c6)oc6ccccc67)c6ccccc6-c6ccccc6)cc5c4c3o2)cc1]

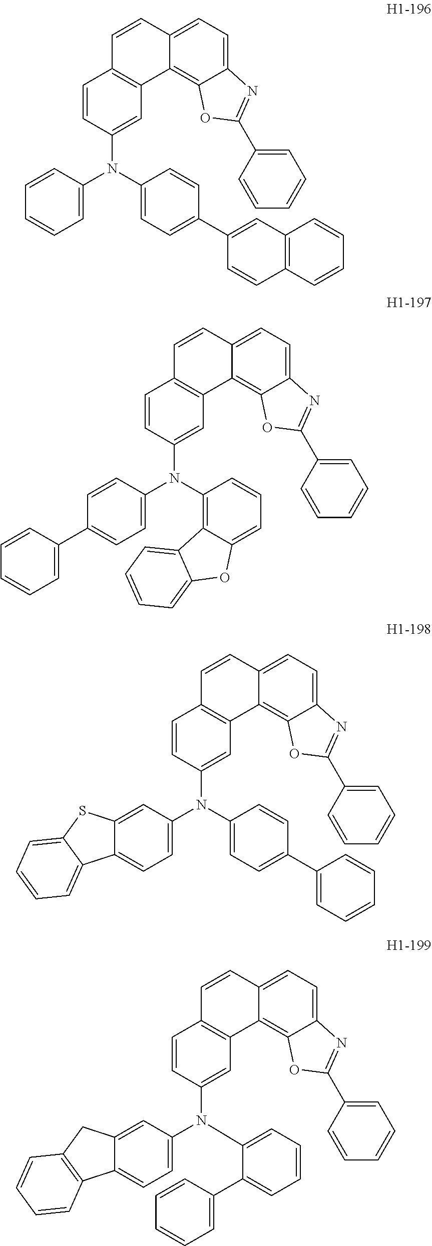

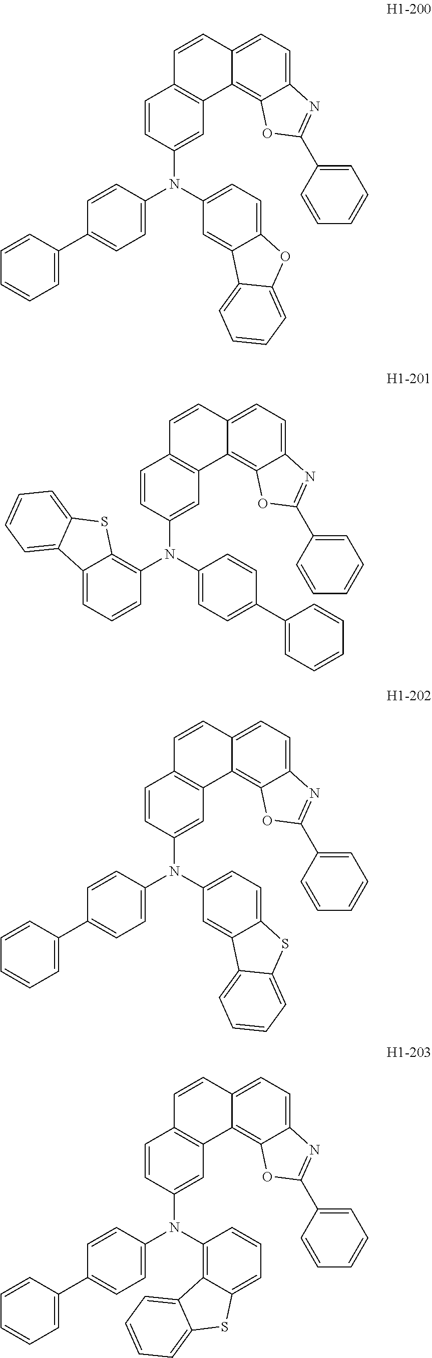

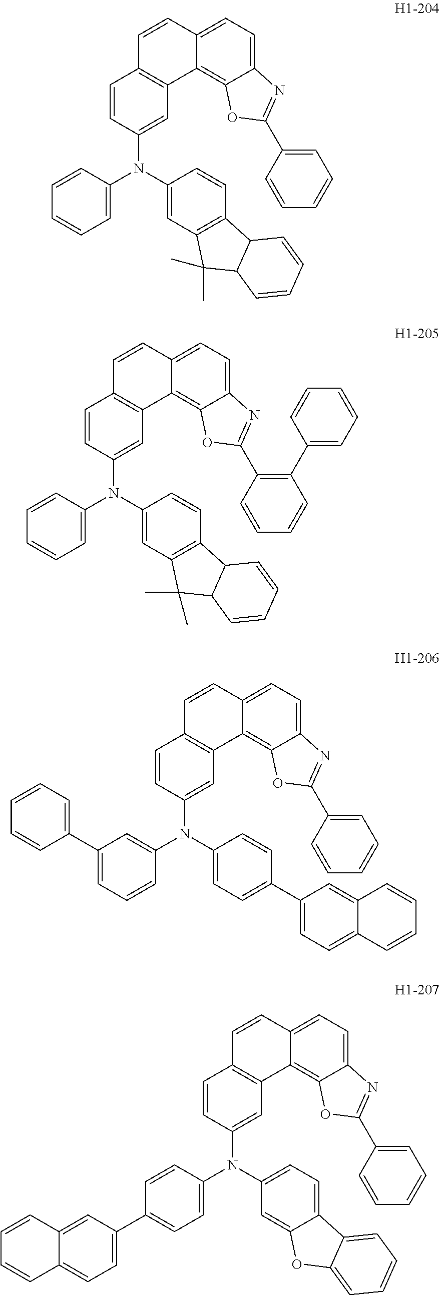

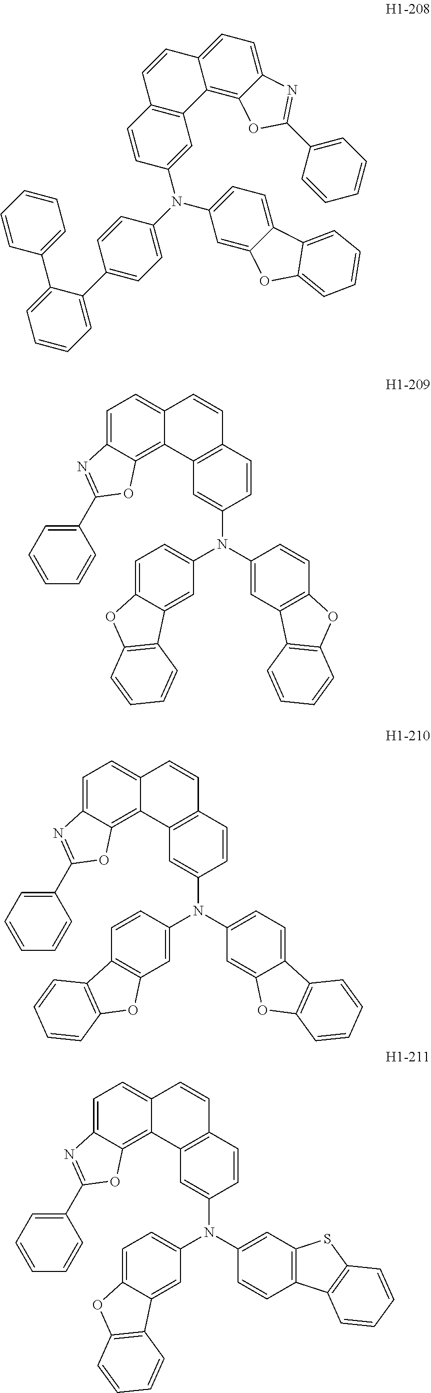 [SMILES: c1ccc(-c2nc3ccc4ccc5ccc(N(c6ccc(-c7ccccc7-c7ccccc7)cc6)c6ccc7c(c6)oc6ccccc67)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1]

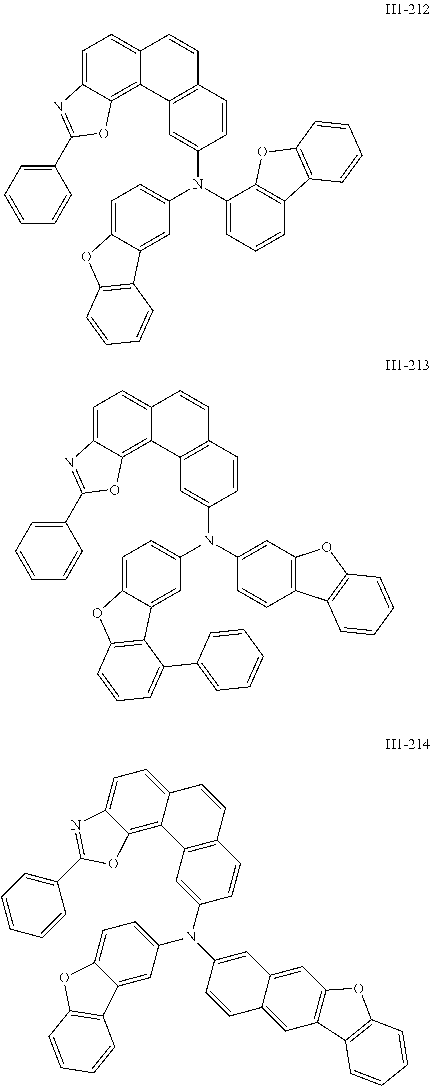 [SMILES: c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1]

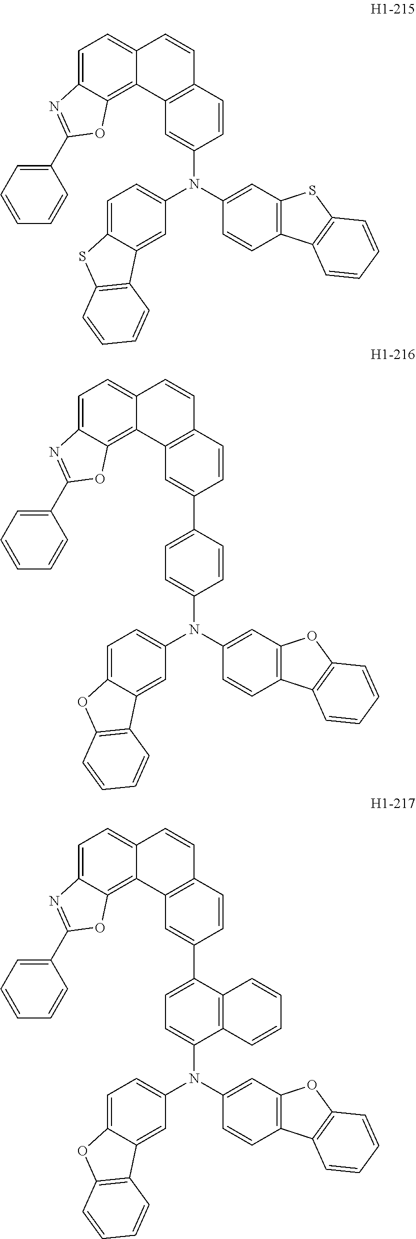

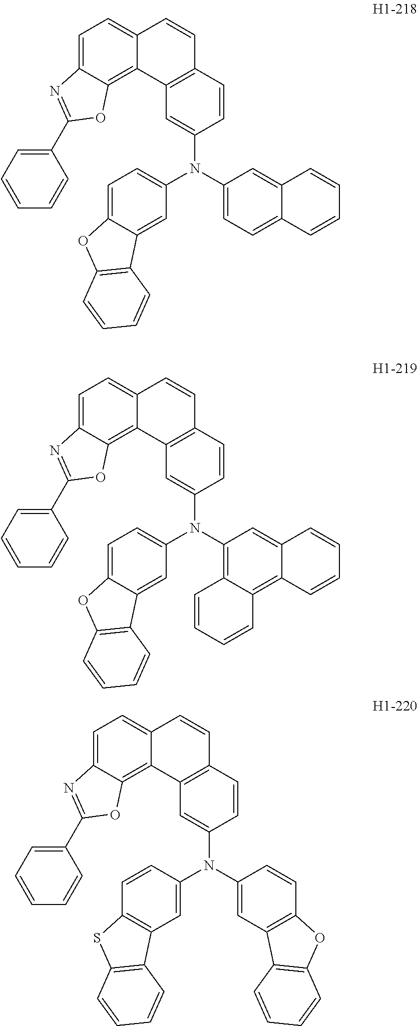

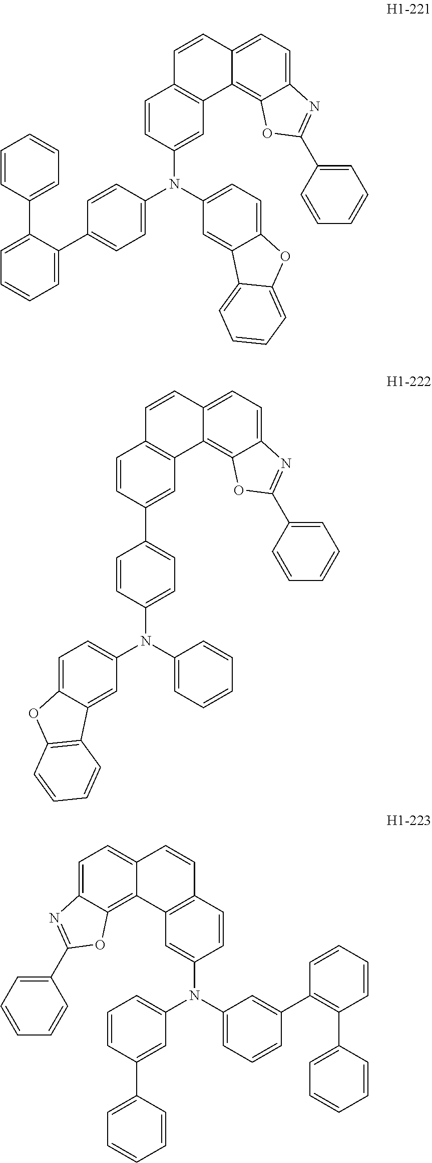 [SMILES: c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1]

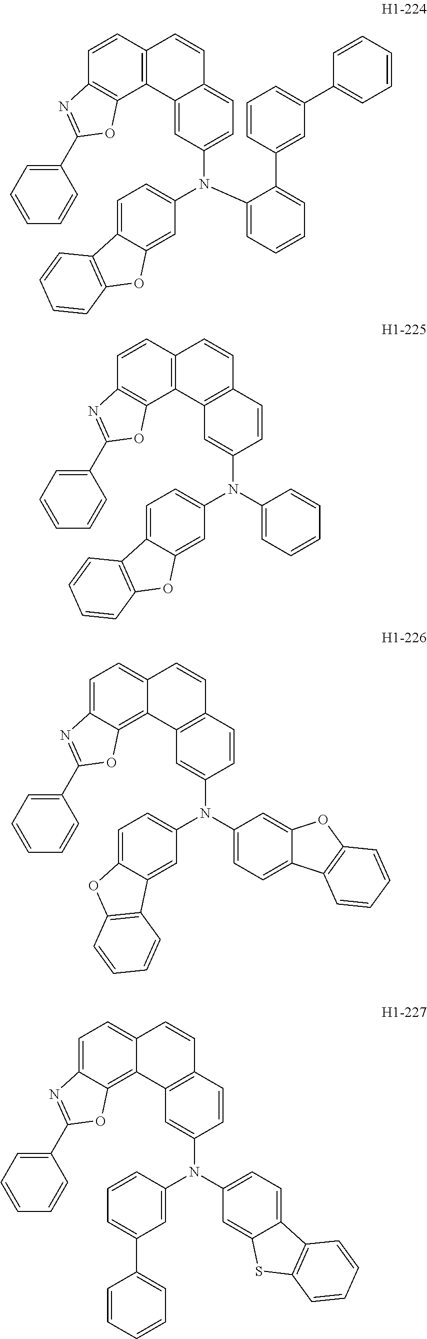

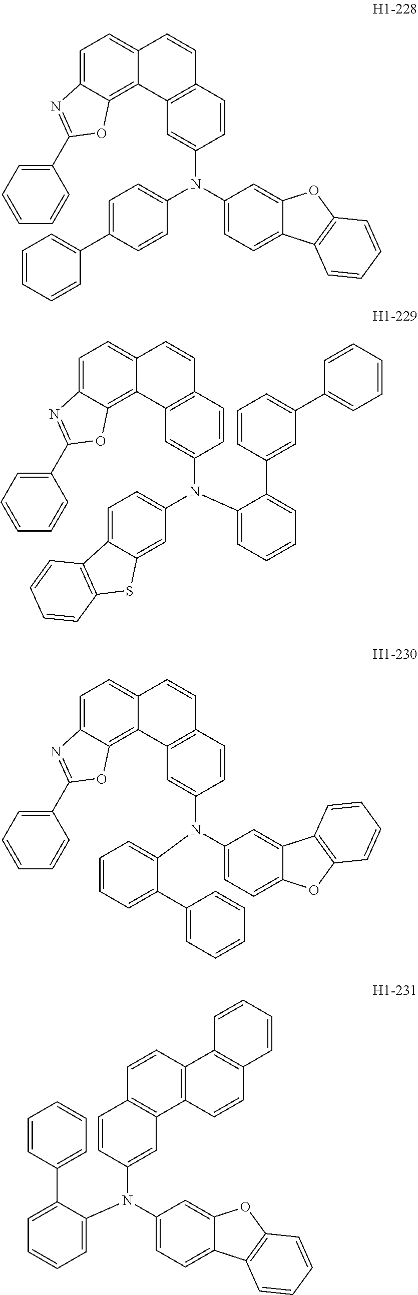 [SMILES: c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2cccc(-c3ccccc3N(c3ccc4c(c3)sc3ccccc34)c3ccc4ccc5ccc6nc(-c7ccccc7)oc6c5c4c3)c2)cc1 ; c1ccc(-c2nc3ccc4ccc5ccc(N(c6ccc7oc8ccccc8c7c6)c6ccccc6-c6ccccc6)cc5c4c3o2)cc1 ; c1ccc(-c2ccccc2N(c2ccc3c(c2)oc2ccccc23)c2ccc3ccc4c5ccccc5ccc4c3c2)cc1]

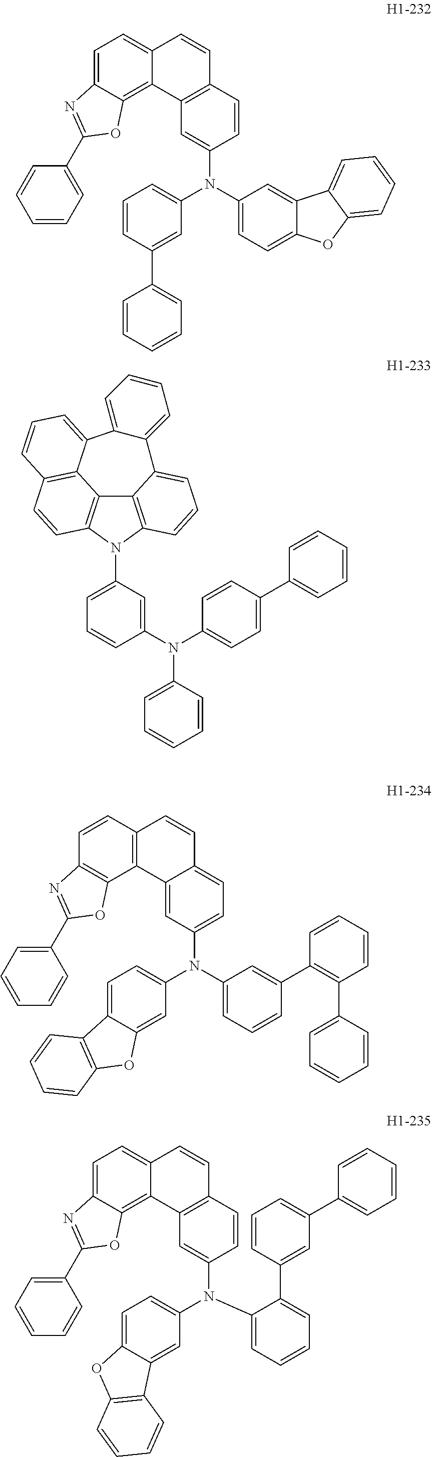

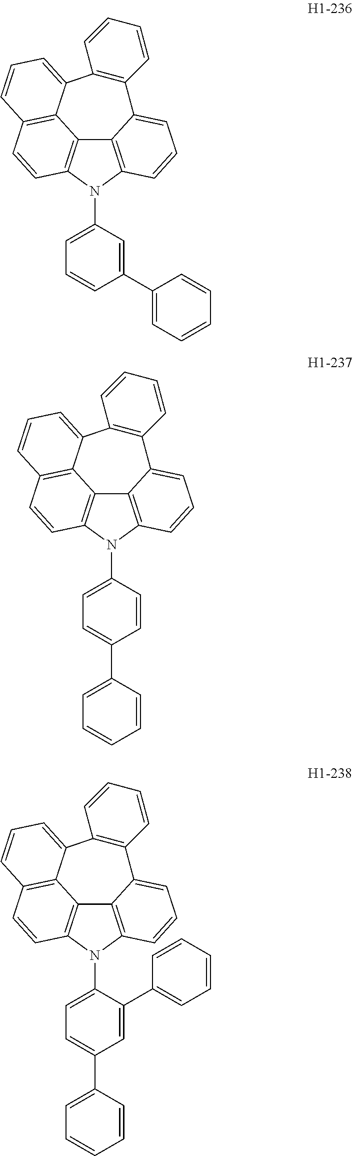

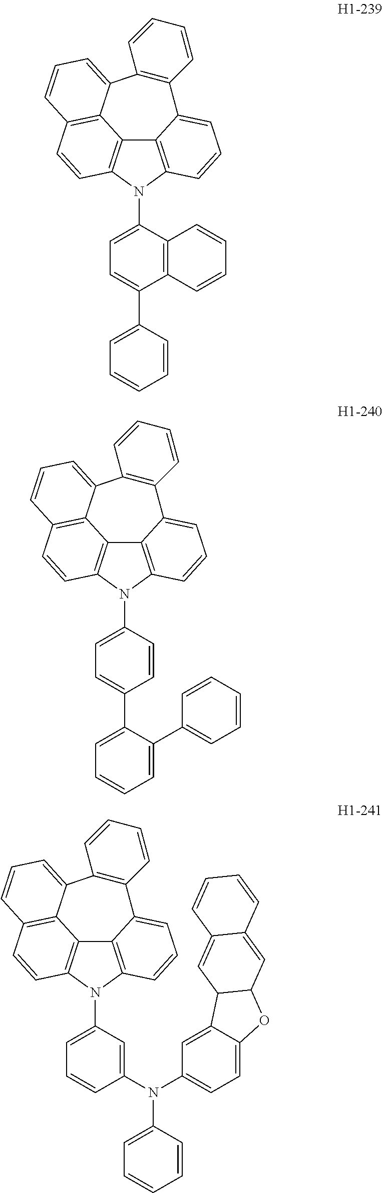

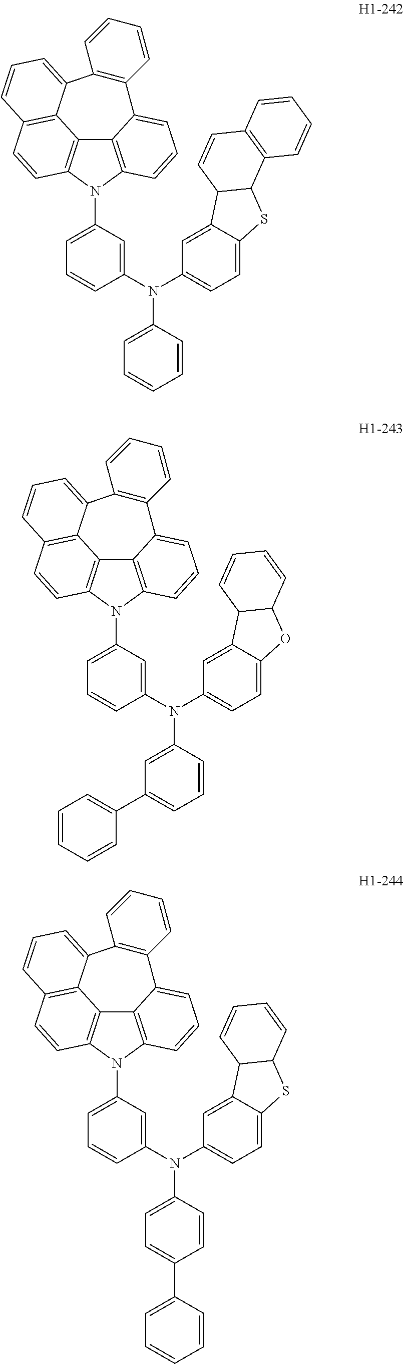 [SMILES: C1=CC2c3cc(N(c4ccccc4)c4cccc(-n5c6cccc7c6c6c8c(cccc8ccc65)-c5ccccc5-7)c4)ccc3SC2c2ccccc21 ; C1=CC2Oc3ccc(N(c4cccc(-c5ccccc5)c4)c4cccc(-n5c6cccc7c6c6c8c(cccc8ccc65)-c5ccccc5-7)c4)cc3C2C=C1 ; C1=CC2Sc3ccc(N(c4ccc(-c5ccccc5)cc4)c4cccc(-n5c6cccc7c6c6c8c(cccc8ccc65)-c5ccccc5-7)c4)cc3C2C=C1]

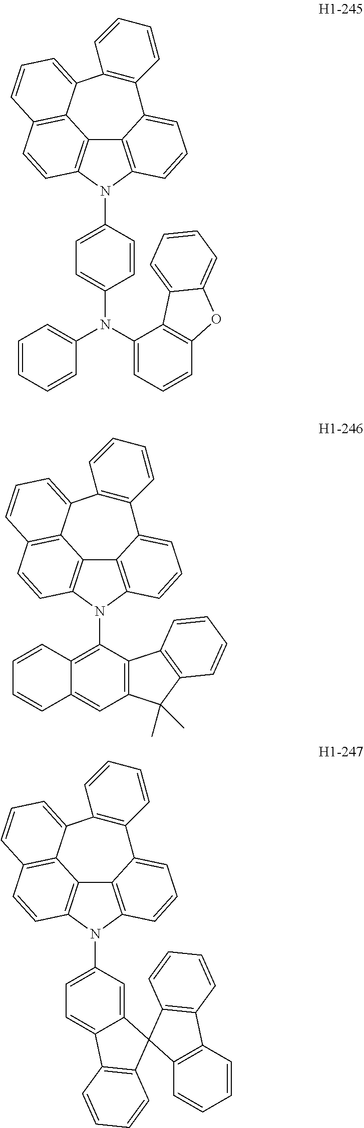

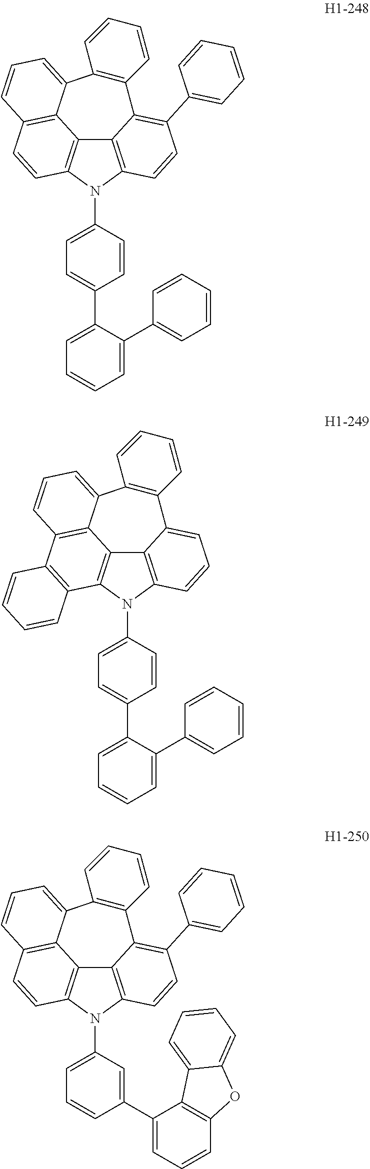

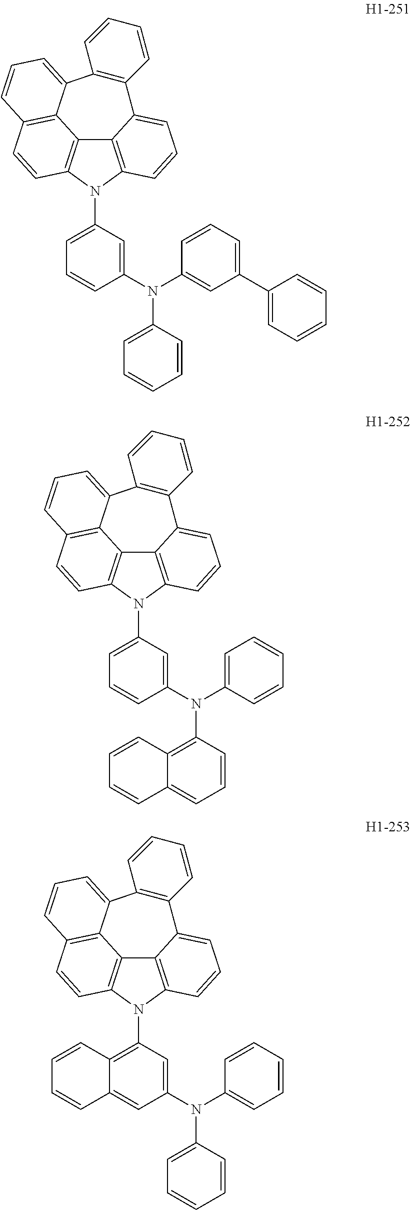

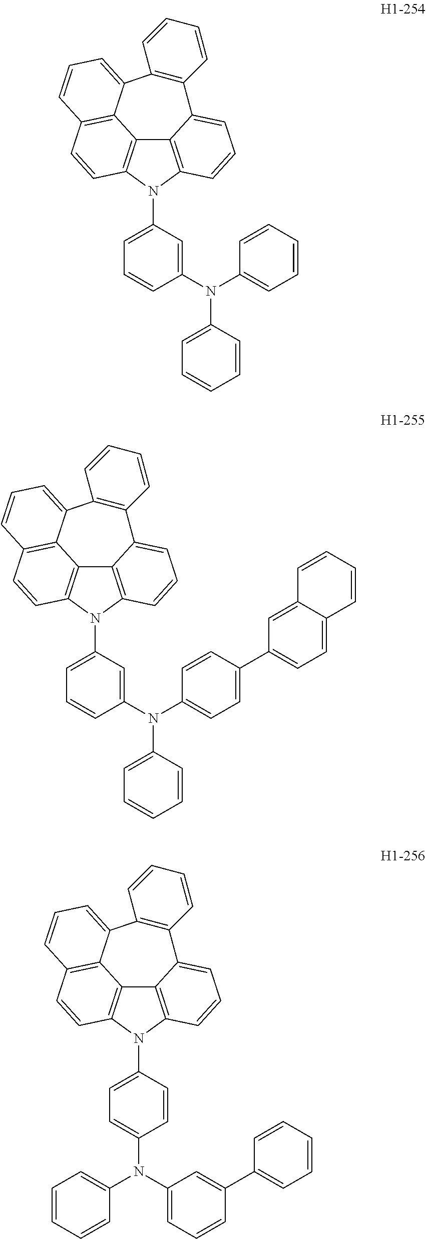 [SMILES: c1ccc(N(c2ccccc2)c2cccc(-n3c4cccc5c4c4c6c(cccc6ccc43)-c3ccccc3-5)c2)cc1 ; c1ccc(N(c2ccc(-c3ccc4ccccc4c3)cc2)c2cccc(-n3c4cccc5c4c4c6c(cccc6ccc43)-c3ccccc3-5)c2)cc1 ; c1ccc(-c2cccc(N(c3ccccc3)c3ccc(-n4c5cccc6c5c5c7c(cccc7ccc54)-c4ccccc4-6)cc3)c2)cc1]

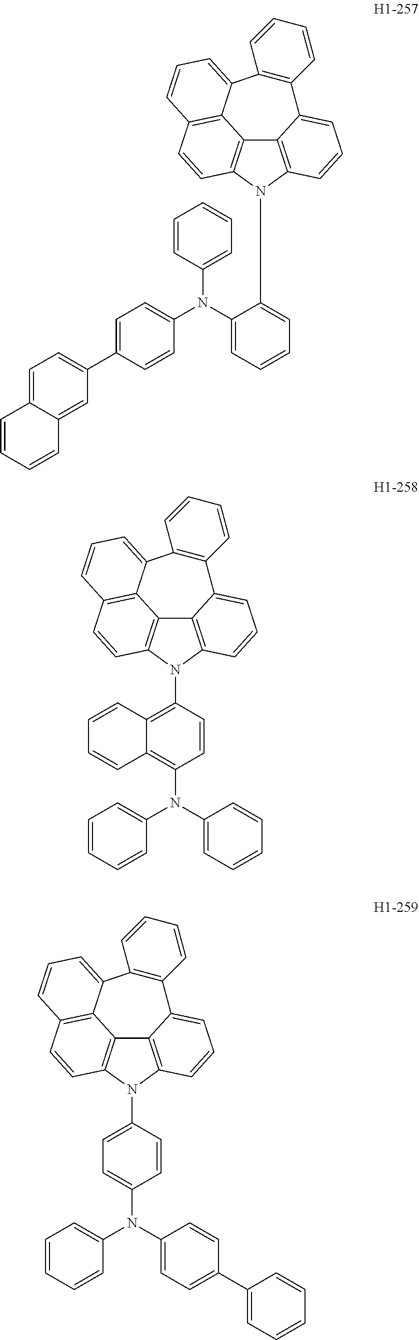 [SMILES: c1ccc(N(c2ccc(-c3ccc4ccccc4c3)cc2)c2ccccc2-n2c3cccc4c3c3c5c(cccc5ccc32)-c2ccccc2-4)cc1 ; c1ccc(N(c2ccccc2)c2ccc(-n3c4cccc5c4c4c6c(cccc6ccc43)-c3ccccc3-5)c3ccccc23)cc1 ; c1ccc(-c2ccc(N(c3ccccc3)c3ccc(-n4c5cccc6c5c5c7c(cccc7ccc54)-c4ccccc4-6)cc3)cc2)cc1]

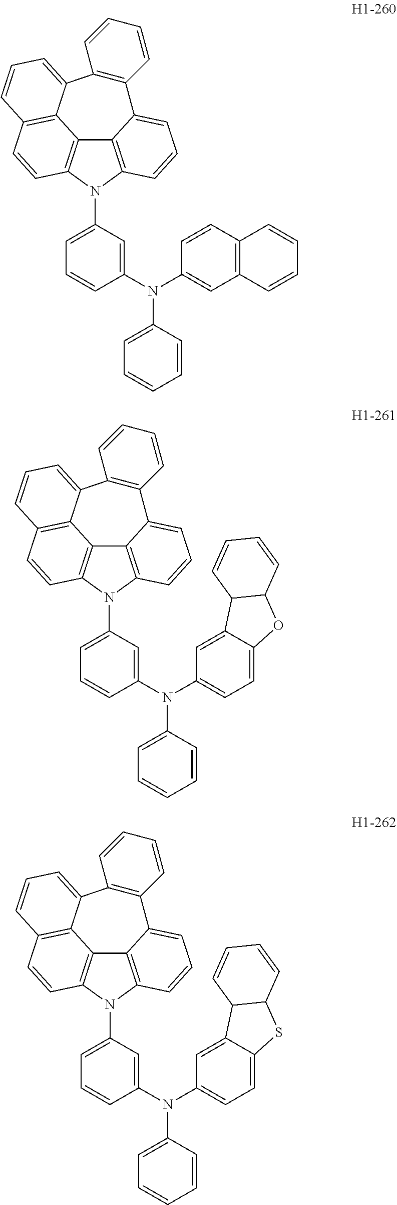

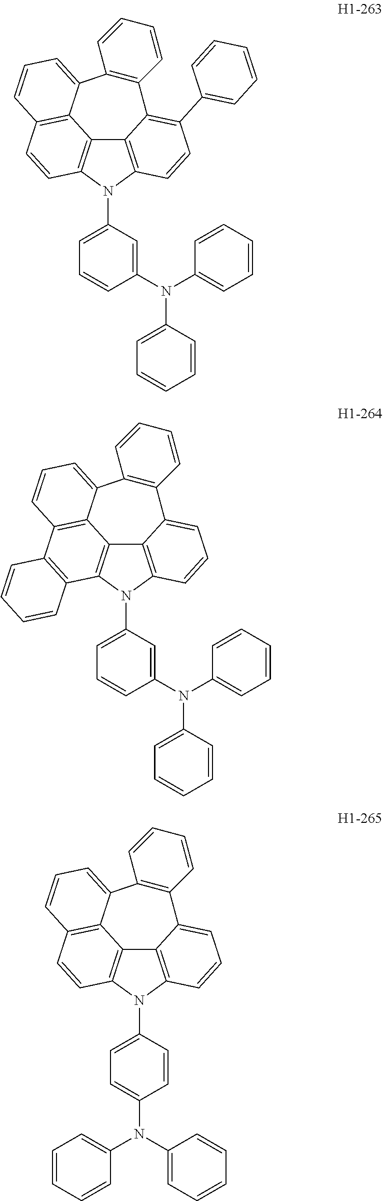

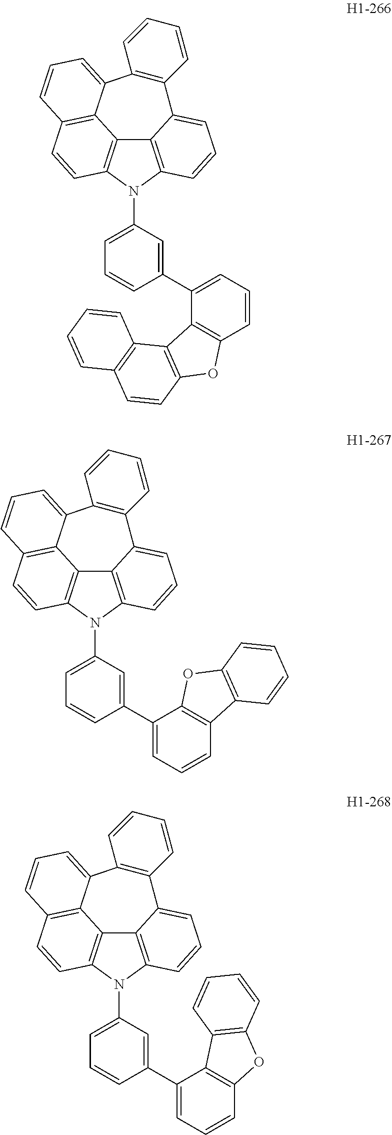 [SMILES: c1cc(-c2cccc3oc4ccc5ccccc5c4c23)cc(-n2c3cccc4c3c3c5c(cccc5ccc32)-c2ccccc2-4)c1 ; c1cc(-c2cccc3c2oc2ccccc23)cc(-n2c3cccc4c3c3c5c(cccc5ccc32)-c2ccccc2-4)c1 ; c1cc(-c2cccc3oc4ccccc4c23)cc(-n2c3cccc4c3c3c5c(cccc5ccc32)-c2ccccc2-4)c1]

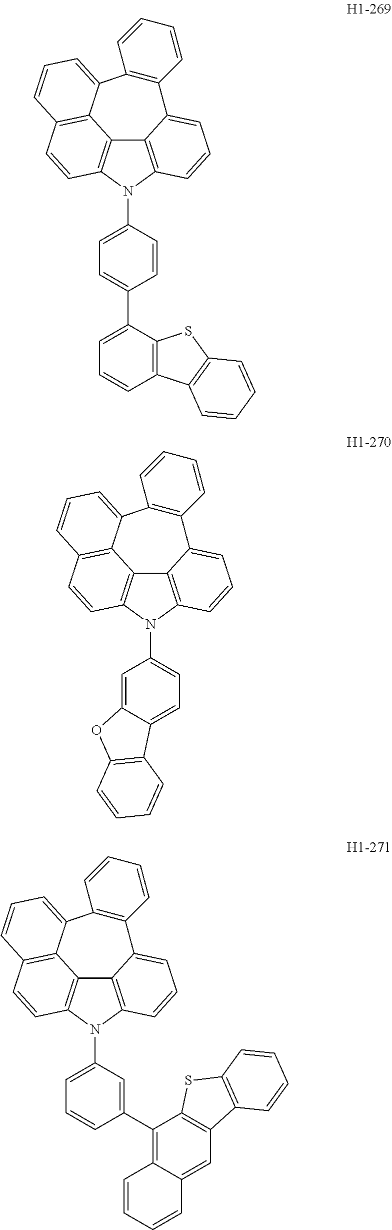

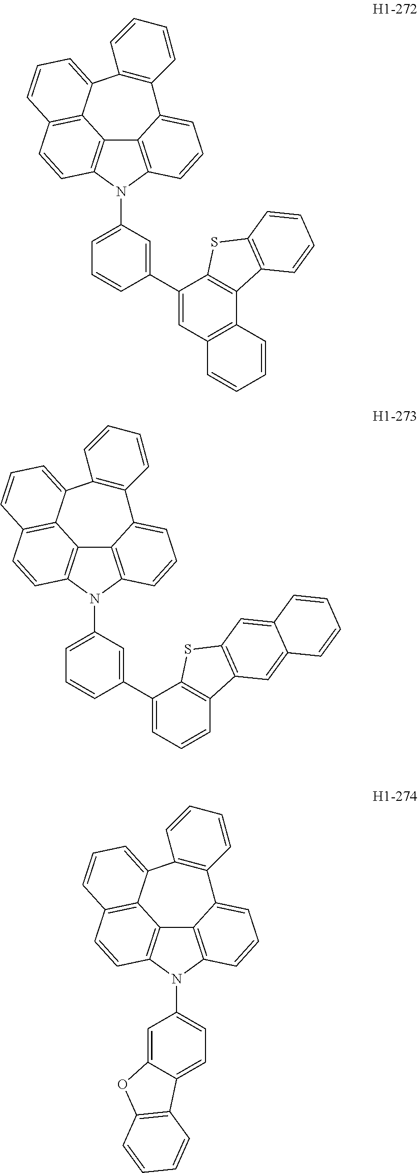 [SMILES: c1cc(-c2cc3ccccc3c3c2sc2ccccc23)cc(-n2c3cccc4c3c3c5c(cccc5ccc32)-c2ccccc2-4)c1 ; c1cc(-c2cccc3c2sc2cc4ccccc4cc23)cc(-n2c3cccc4c3c3c5c(cccc5ccc32)-c2ccccc2-4)c1 ; c1ccc2c(c1)-c1cccc3ccc4c(c13)c1c-2cccc1n4-c1ccc2c(c1)oc1ccccc12]

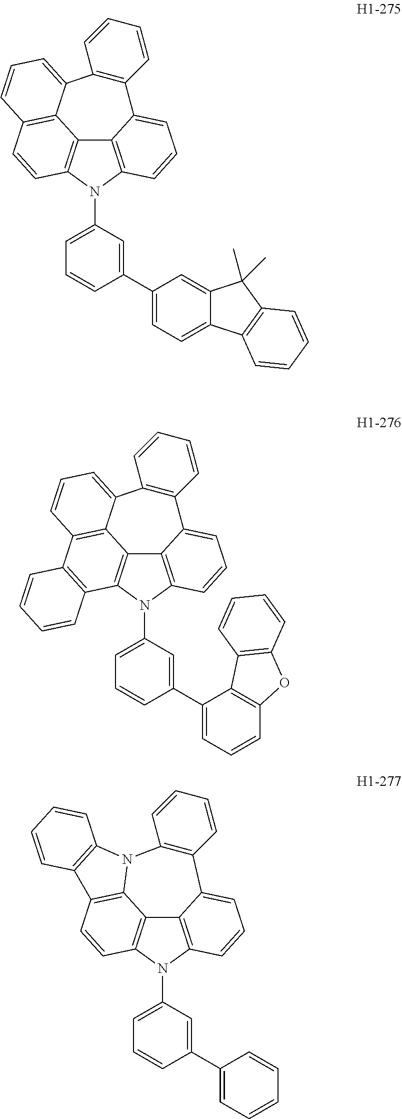 [SMILES: CC1(C)c2ccccc2-c2ccc(-c3cccc(-n4c5cccc6c5c5c7c(cccc7ccc54)-c4ccccc4-6)c3)cc21 ; c1cc(-c2cccc3oc4ccccc4c23)cc(-n2c3cccc4c3c3c5c(cccc5c5ccccc5c32)-c2ccccc2-4)c1 ; c1ccc(-c2cccc(-n3c4cccc5c6ccccc6n6c7ccccc7c7ccc3c(c54)c76)c2)cc1]

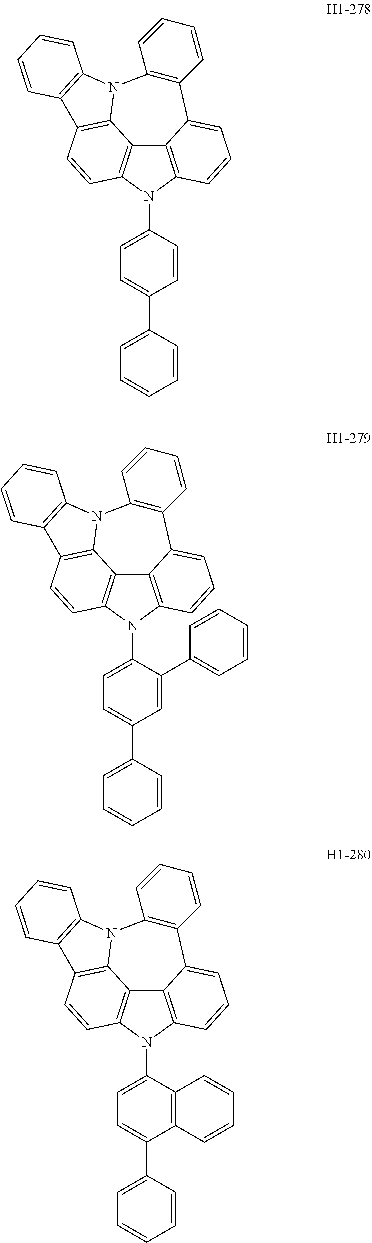 [SMILES: c1ccc(-c2ccc(-n3c4cccc5c6ccccc6n6c7ccccc7c7ccc3c(c54)c76)cc2)cc1 ; c1ccc(-c2ccc(-n3c4cccc5c6ccccc6n6c7ccccc7c7ccc3c(c54)c76)c(-c3ccccc3)c2)cc1 ; c1ccc(-c2ccc(-n3c4cccc5c6ccccc6n6c7ccccc7c7ccc3c(c54)c76)c3ccccc23)cc1]

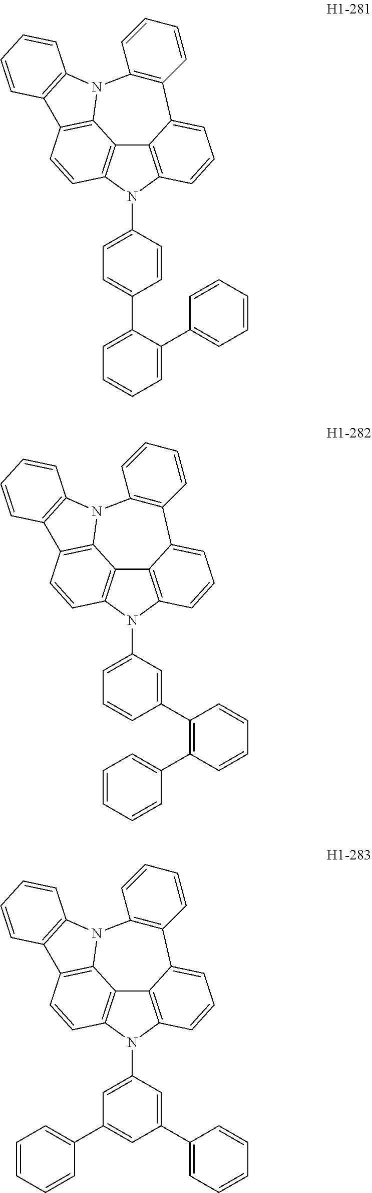

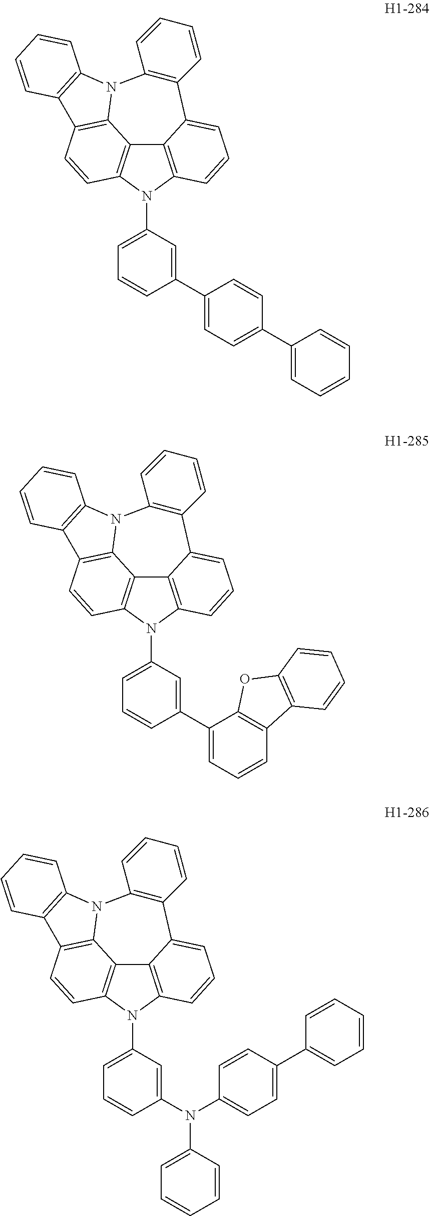 [SMILES: c1ccc(-c2ccc(-c3cccc(-n4c5cccc6c7ccccc7n7c8ccccc8c8ccc4c(c65)c87)c3)cc2)cc1 ; c1cc(-c2cccc3c2oc2ccccc23)cc(-n2c3cccc4c5ccccc5n5c6ccccc6c6ccc2c(c43)c65)c1 ; c1ccc(-c2ccc(N(c3ccccc3)c3cccc(-n4c5cccc6c7ccccc7n7c8ccccc8c8ccc4c(c65)c87)c3)cc2)cc1]

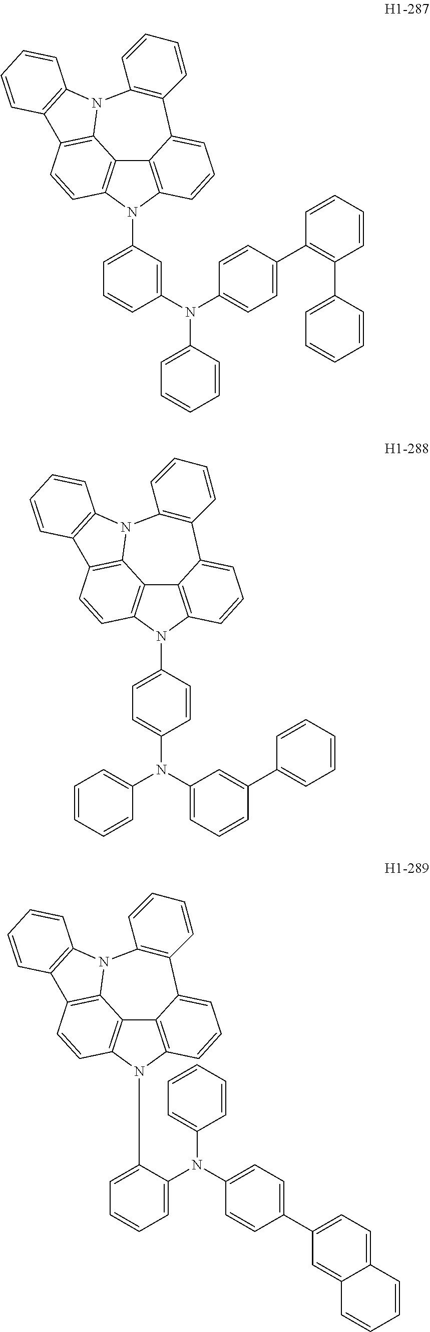 [SMILES: c1ccc(-c2ccccc2-c2ccc(N(c3ccccc3)c3cccc(-n4c5cccc6c7ccccc7n7c8ccccc8c8ccc4c(c65)c87)c3)cc2)cc1 ; c1ccc(-c2cccc(N(c3ccccc3)c3ccc(-n4c5cccc6c7ccccc7n7c8ccccc8c8ccc4c(c65)c87)cc3)c2)cc1 ; c1ccc(N(c2ccc(-c3ccc4ccccc4c3)cc2)c2ccccc2-n2c3cccc4c5ccccc5n5c6ccccc6c6ccc2c(c43)c65)cc1]

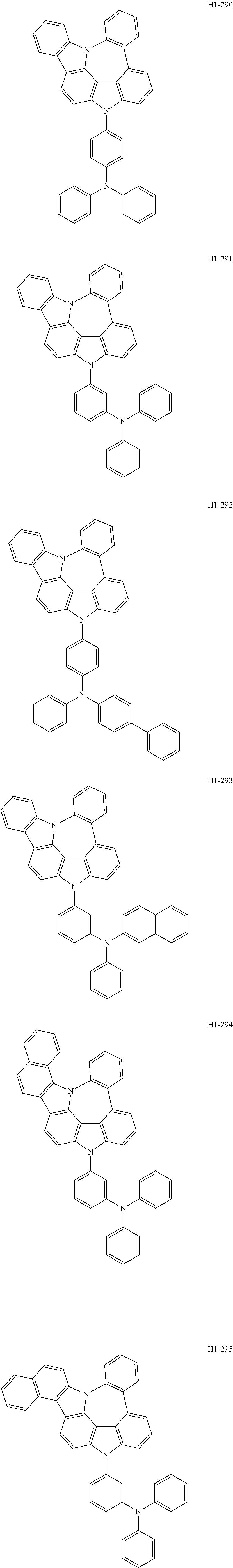 [SMILES: c1ccc(N(c2ccccc2)c2ccc(-n3c4cccc5c6ccccc6n6c7ccccc7c7ccc3c(c54)c76)cc2)cc1 ; c1ccc(N(c2ccccc2)c2cccc(-n3c4cccc5c6ccccc6n6c7ccccc7c7ccc3c(c54)c76)c2)cc1 ; c1ccc(-c2ccc(N(c3ccccc3)c3ccc(-n4c5cccc6c7ccccc7n7c8ccccc8c8ccc4c(c65)c87)cc3)cc2)cc1 ; c1ccc(N(c2cccc(-n3c4cccc5c6ccccc6n6c7ccccc7c7ccc3c(c54)c76)c2)c2ccc3ccccc3c2)cc1 ; c1ccc(N(c2ccccc2)c2cccc(-n3c4cccc5c6ccccc6n6c7c8ccccc8ccc7c7ccc3c(c54)c76)c2)cc1 ; c1ccc(N(c2ccccc2)c2cccc(-n3c4cccc5c6ccccc6n6c7ccc8ccccc8c7c7ccc3c(c54)c76)c2)cc1]

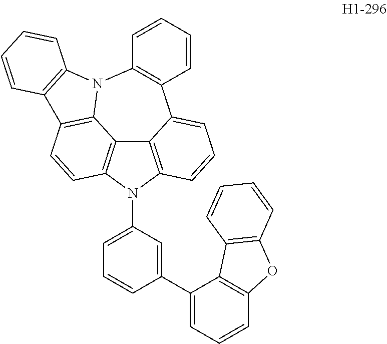 [SMILES: c1cc(-c2cccc3oc4ccccc4c23)cc(-n2c3cccc4c5ccccc5n5c6ccccc6c6ccc2c(c43)c65)c1]

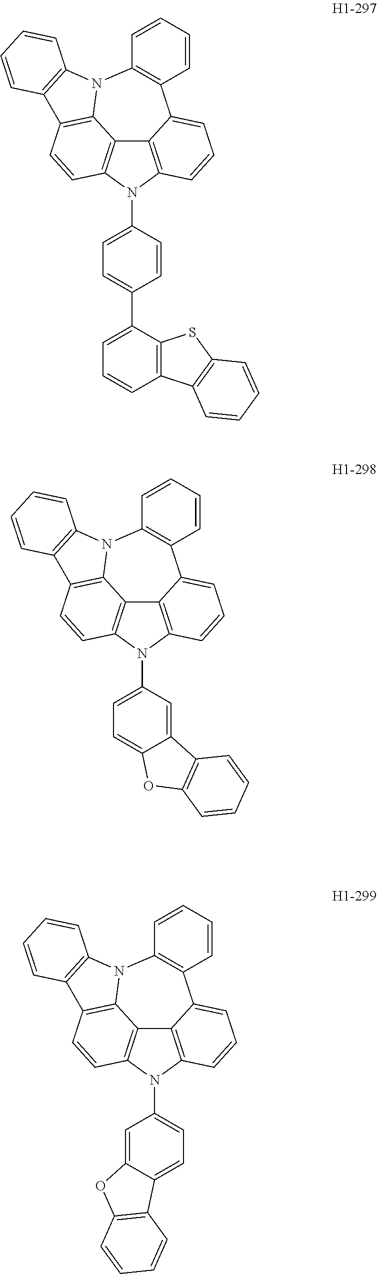 [SMILES: c1ccc2c(c1)sc1c(-c3ccc(-n4c5cccc6c7ccccc7n7c8ccccc8c8ccc4c(c65)c87)cc3)cccc12 ; c1ccc2c(c1)oc1ccc(-n3c4cccc5c6ccccc6n6c7ccccc7c7ccc3c(c54)c76)cc12 ; c1ccc2c(c1)oc1cc(-n3c4cccc5c6ccccc6n6c7ccccc7c7ccc3c(c54)c76)ccc12]

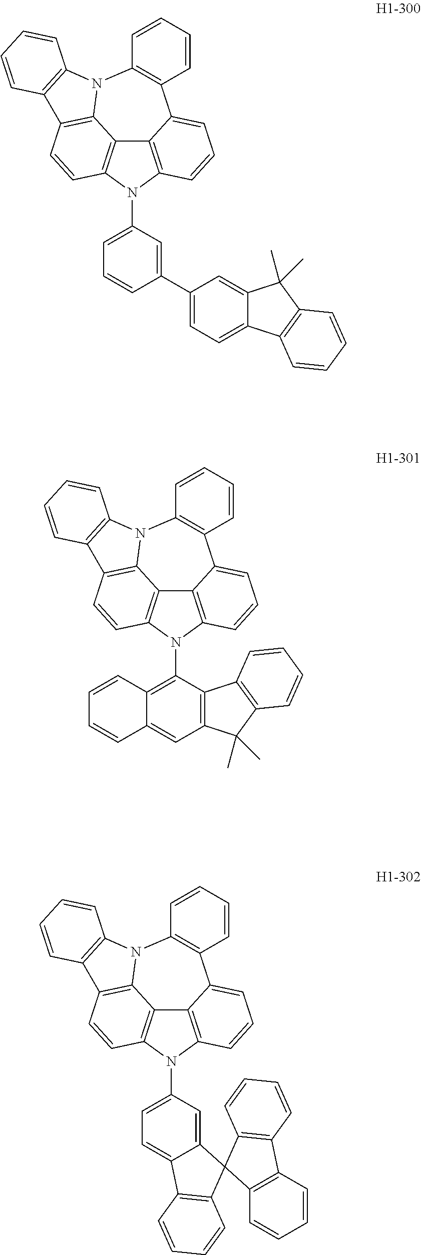 [SMILES: CC1(C)c2ccccc2-c2ccc(-c3cccc(-n4c5cccc6c7ccccc7n7c8ccccc8c8ccc4c(c65)c87)c3)cc21 ; CC1(C)c2ccccc2-c2c1cc1ccccc1c2-n1c2cccc3c4ccccc4n4c5ccccc5c5ccc1c(c32)c54 ; c1ccc2c(c1)-c1ccccc1C21c2ccccc2-c2ccc(-n3c4cccc5c6ccccc6n6c7ccccc7c7ccc3c(c54)c76)cc21]

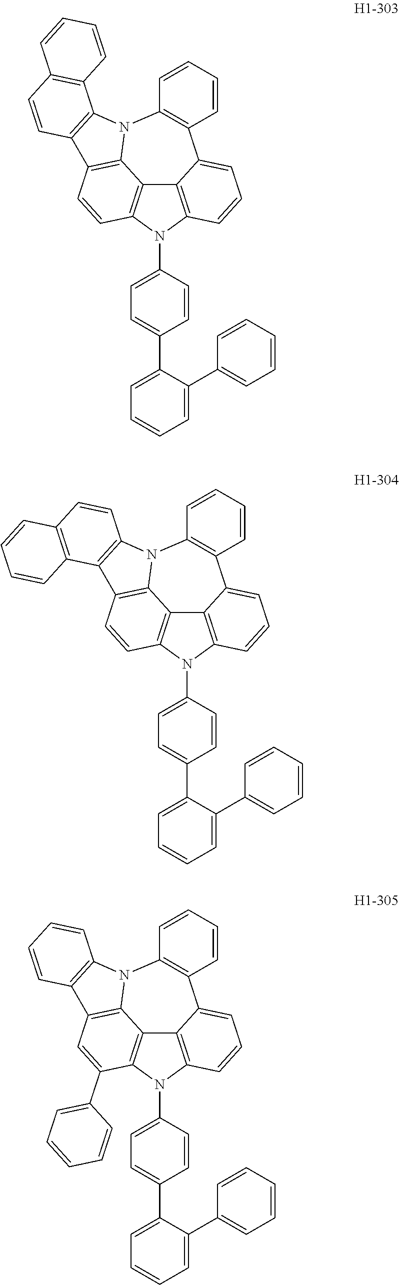

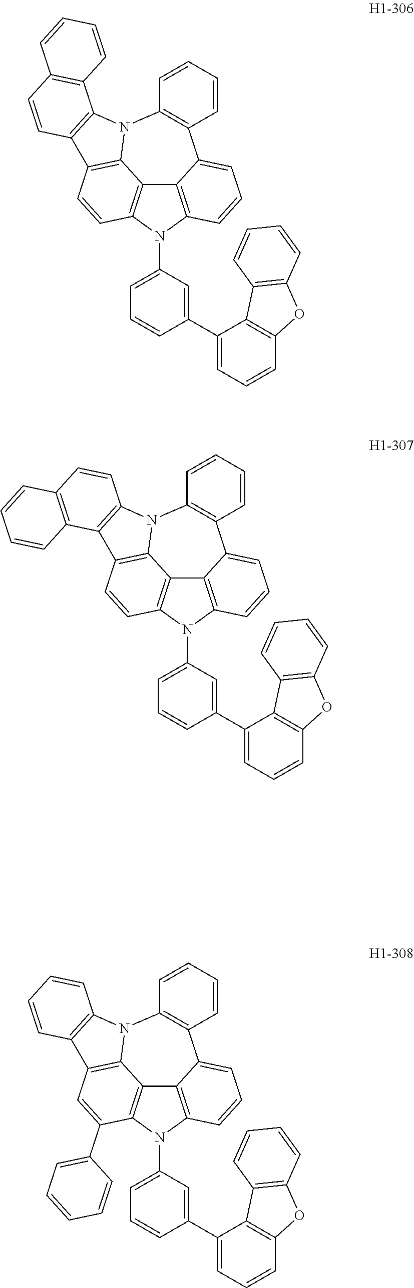 [SMILES: c1cc(-c2cccc3oc4ccccc4c23)cc(-n2c3cccc4c5ccccc5n5c6c7ccccc7ccc6c6ccc2c(c43)c65)c1 ; c1cc(-c2cccc3oc4ccccc4c23)cc(-n2c3cccc4c5ccccc5n5c6ccc7ccccc7c6c6ccc2c(c43)c65)c1 ; c1ccc(-c2cc3c4ccccc4n4c5ccccc5c5cccc6c5c(c2n6-c2cccc(-c5cccc6oc7ccccc7c56)c2)c34)cc1]

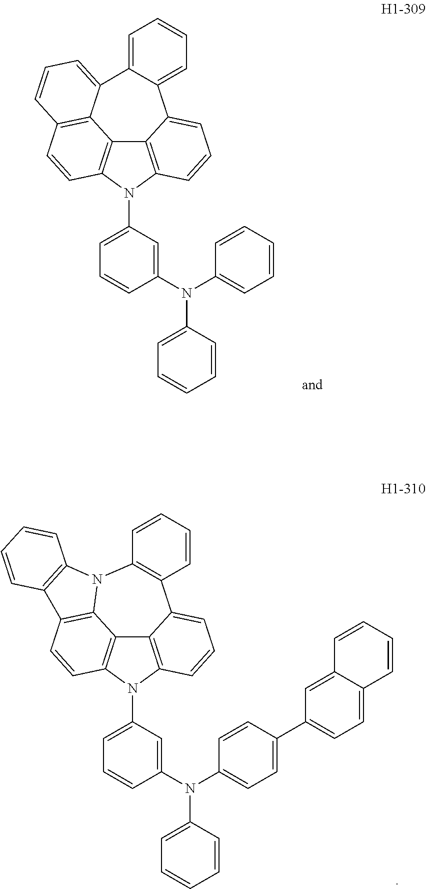 [SMILES: c1ccc(N(c2ccccc2)c2cccc(-n3c4cccc5c4c4c6c(cccc6ccc43)-c3ccccc3-5)c2)cc1 ; c1ccc(N(c2ccc(-c3ccc4ccccc4c3)cc2)c2cccc(-n3c4cccc5c6ccccc6n6c7ccccc7c7ccc3c(c54)c76)c2)cc1]

11. The plurality of host materials according to claim 2, wherein the compound represented by any one of the formulas 2 to 4 is at least one selected from the group consisting of the following compounds:

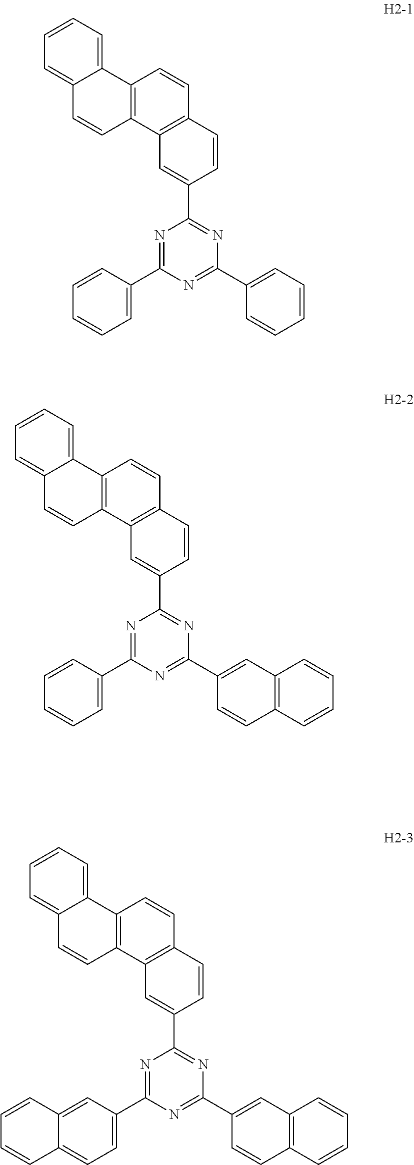 [SMILES: c1ccc(-c2nc(-c3ccccc3)nc(-c3ccc4ccc5c6ccccc6ccc5c4c3)n2)cc1 ; c1ccc(-c2nc(-c3ccc4ccccc4c3)nc(-c3ccc4ccc5c6ccccc6ccc5c4c3)n2)cc1 ; c1ccc2cc(-c3nc(-c4ccc5ccccc5c4)nc(-c4ccc5ccc6c7ccccc7ccc6c5c4)n3)ccc2c1]

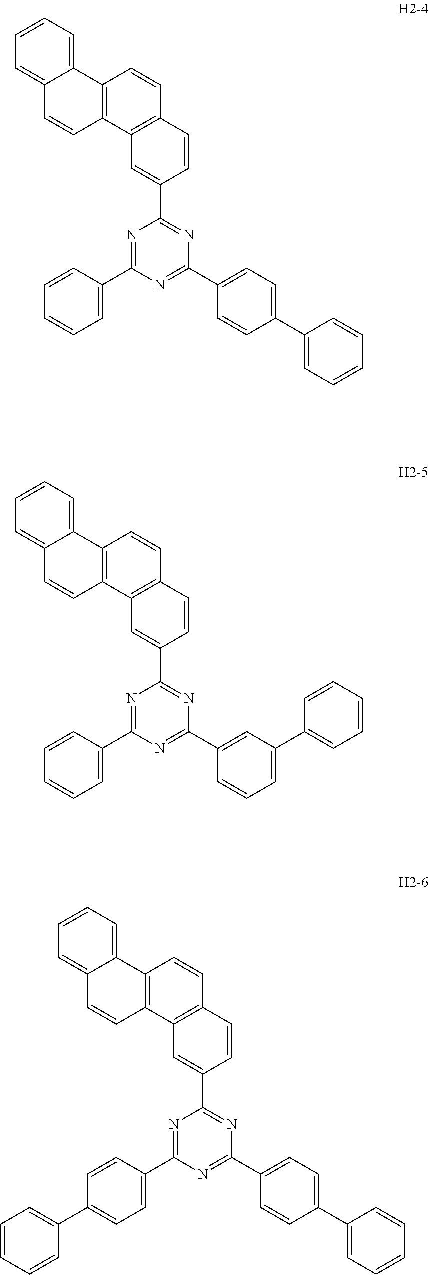 [SMILES: c1ccc(-c2ccc(-c3nc(-c4ccccc4)nc(-c4ccc5ccc6c7ccccc7ccc6c5c4)n3)cc2)cc1 ; c1ccc(-c2cccc(-c3nc(-c4ccccc4)nc(-c4ccc5ccc6c7ccccc7ccc6c5c4)n3)c2)cc1 ; c1ccc(-c2ccc(-c3nc(-c4ccc(-c5ccccc5)cc4)nc(-c4ccc5ccc6c7ccccc7ccc6c5c4)n3)cc2)cc1]

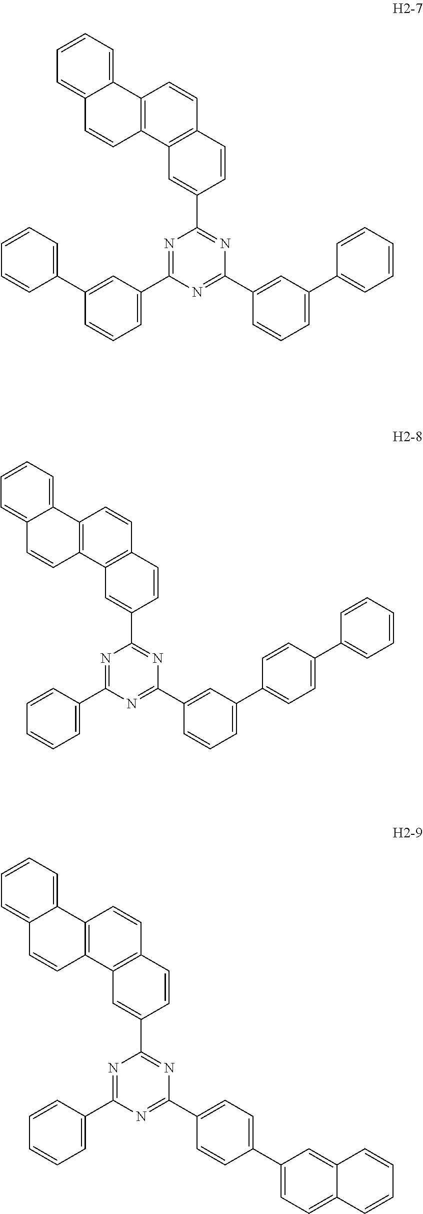 [SMILES: c1ccc(-c2cccc(-c3nc(-c4cccc(-c5ccccc5)c4)nc(-c4ccc5ccc6c7ccccc7ccc6c5c4)n3)c2)cc1 ; c1ccc(-c2ccc(-c3cccc(-c4nc(-c5ccccc5)nc(-c5ccc6ccc7c8ccccc8ccc7c6c5)n4)c3)cc2)cc1 ; c1ccc(-c2nc(-c3ccc(-c4ccc5ccccc5c4)cc3)nc(-c3ccc4ccc5c6ccccc6ccc5c4c3)n2)cc1]

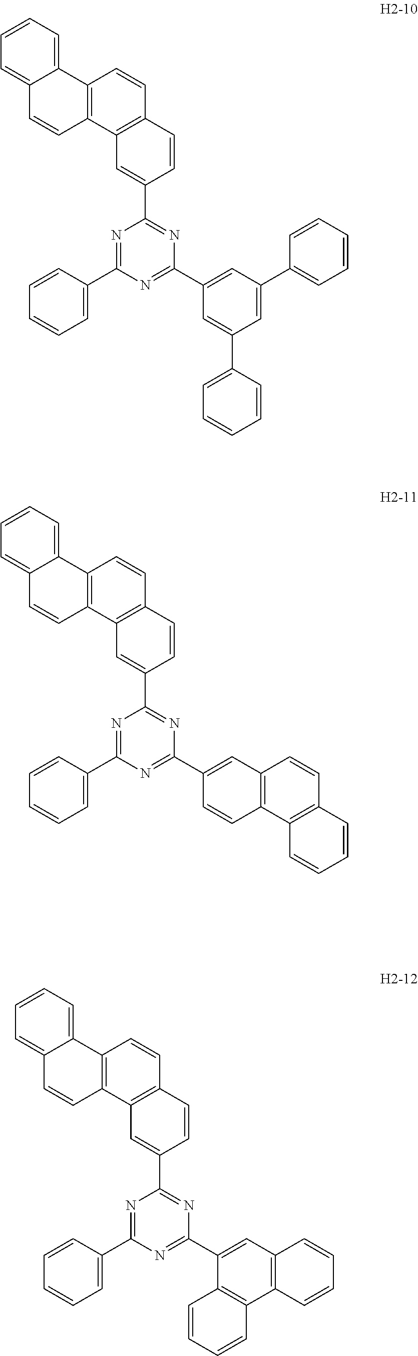 [SMILES: c1ccc(-c2cc(-c3ccccc3)cc(-c3nc(-c4ccccc4)nc(-c4ccc5ccc6c7ccccc7ccc6c5c4)n3)c2)cc1 ; c1ccc(-c2nc(-c3ccc4c(ccc5ccccc54)c3)nc(-c3ccc4ccc5c6ccccc6ccc5c4c3)n2)cc1 ; c1ccc(-c2nc(-c3ccc4ccc5c6ccccc6ccc5c4c3)nc(-c3cc4ccccc4c4ccccc34)n2)cc1]

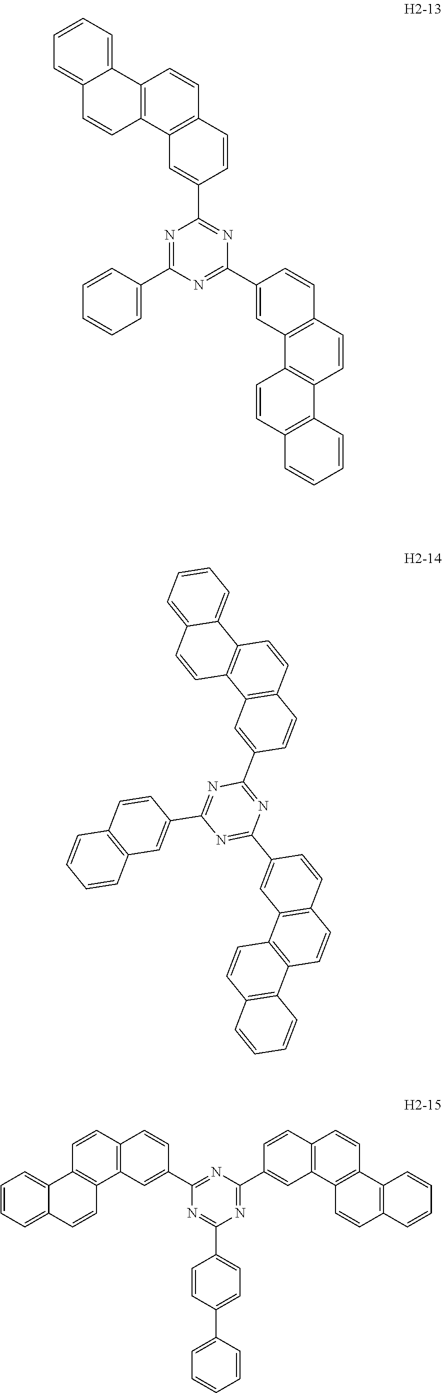 [SMILES: c1ccc(-c2nc(-c3ccc4ccc5c6ccccc6ccc5c4c3)nc(-c3ccc4ccc5c6ccccc6ccc5c4c3)n2)cc1 ; c1ccc2cc(-c3nc(-c4ccc5ccc6c7ccccc7ccc6c5c4)nc(-c4ccc5ccc6c7ccccc7ccc6c5c4)n3)ccc2c1 ; c1ccc(-c2ccc(-c3nc(-c4ccc5ccc6c7ccccc7ccc6c5c4)nc(-c4ccc5ccc6c7ccccc7ccc6c5c4)n3)cc2)cc1]

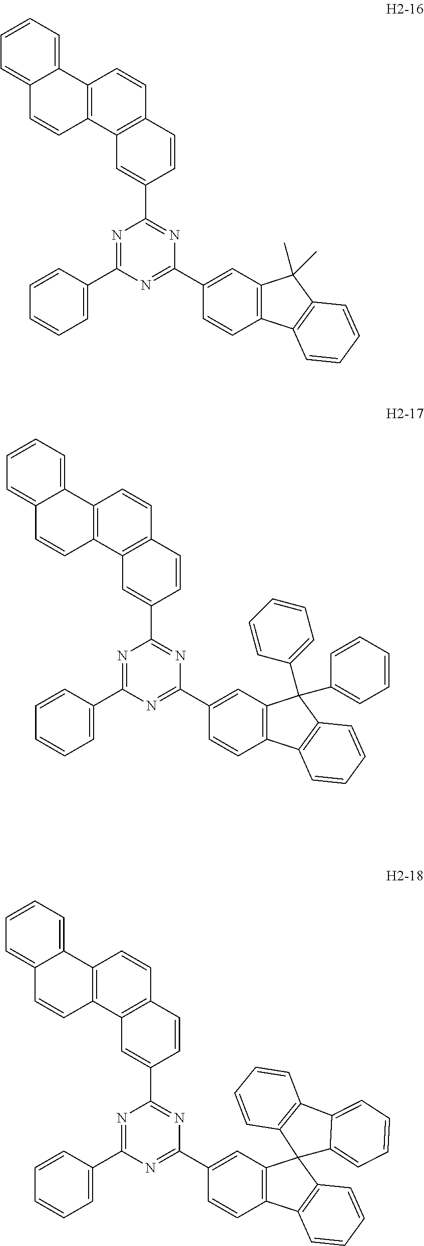 [SMILES: CC1(C)c2ccccc2-c2ccc(-c3nc(-c4ccccc4)nc(-c4ccc5ccc6c7ccccc7ccc6c5c4)n3)cc21 ; c1ccc(-c2nc(-c3ccc4c(c3)C(c3ccccc3)(c3ccccc3)c3ccccc3-4)nc(-c3ccc4ccc5c6ccccc6ccc5c4c3)n2)cc1 ; c1ccc(-c2nc(-c3ccc4c(c3)C3(c5ccccc5-c5ccccc53)c3ccccc3-4)nc(-c3ccc4ccc5c6ccccc6ccc5c4c3)n2)cc1]

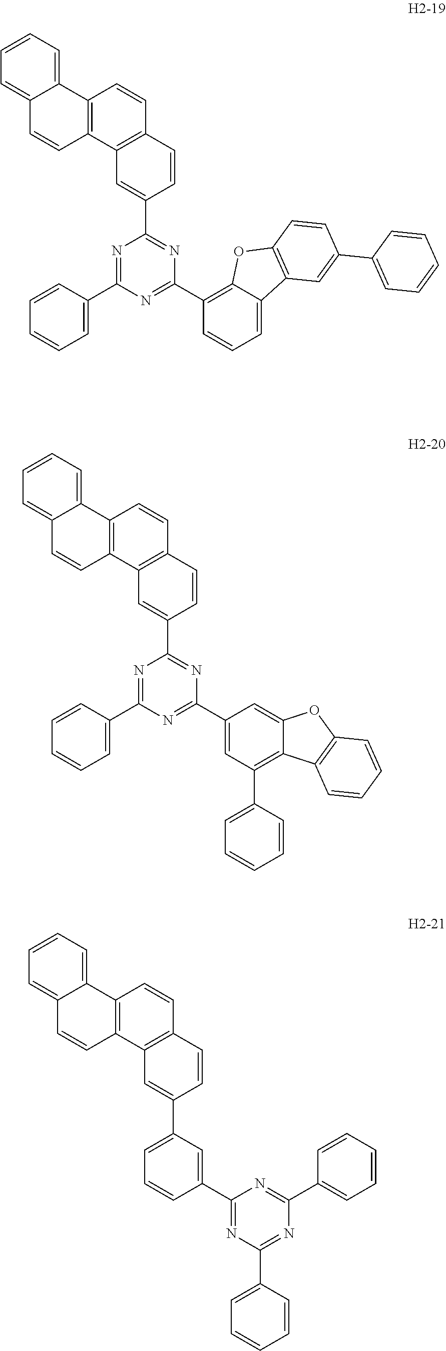 [SMILES: c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccccc3)nc(-c3cccc(-c4ccc5ccc6c7ccccc7ccc6c5c4)c3)n2)cc1]

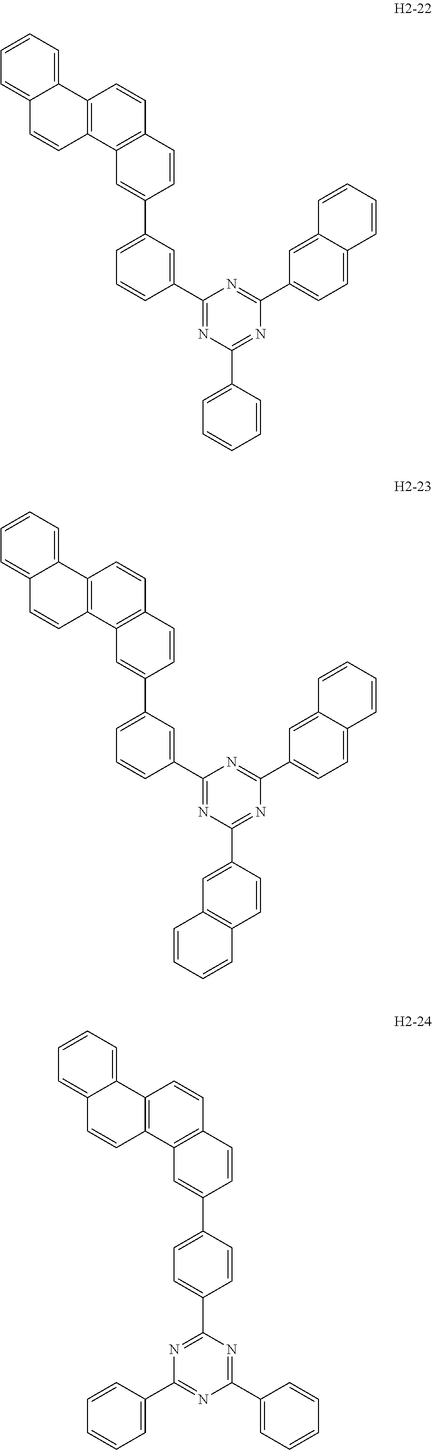 [SMILES: c1ccc(-c2nc(-c3cccc(-c4ccc5ccc6c7ccccc7ccc6c5c4)c3)nc(-c3ccc4ccccc4c3)n2)cc1 ; c1cc(-c2ccc3ccc4c5ccccc5ccc4c3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc6c7ccccc7ccc6c5c4)cc3)n2)cc1]

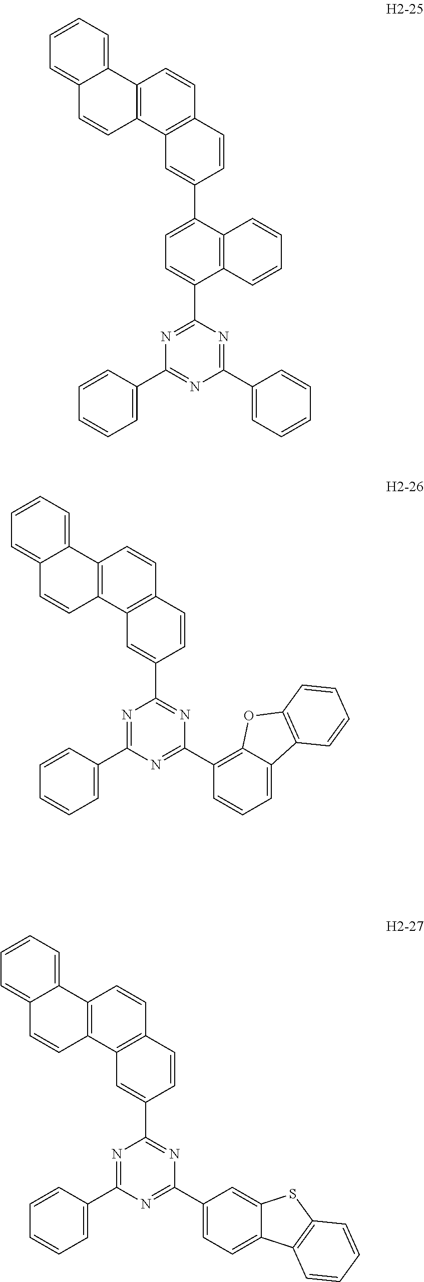 [SMILES: c1ccc(-c2nc(-c3ccccc3)nc(-c3ccc(-c4ccc5ccc6c7ccccc7ccc6c5c4)c4ccccc34)n2)cc1 ; c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1]

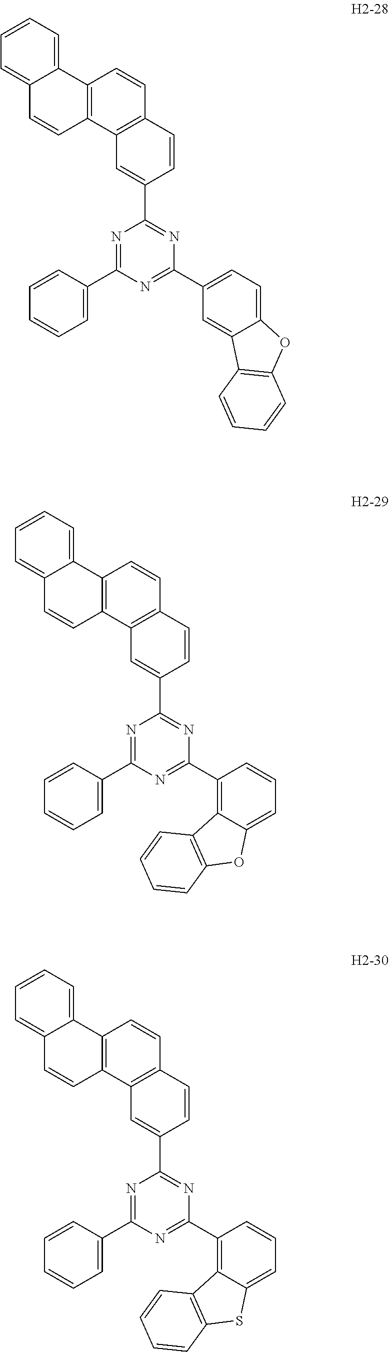 [SMILES: c1ccc(-c2nc(-c3ccc4ccc5c6ccccc6ccc5c4c3)nc(-c3ccc4oc5ccccc5c4c3)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1]

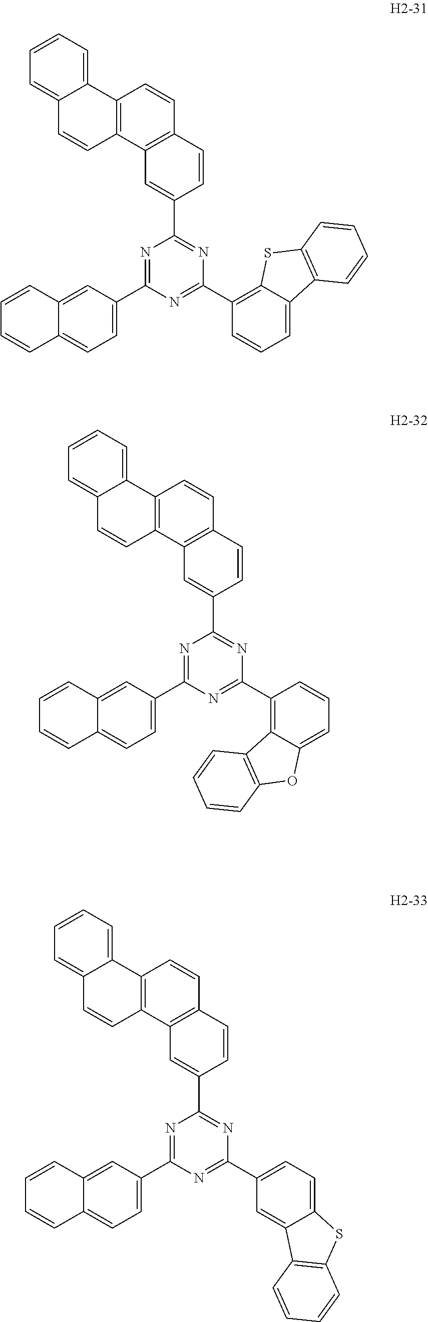 [SMILES: c1ccc2cc(-c3nc(-c4ccc5ccc6c7ccccc7ccc6c5c4)nc(-c4cccc5c4sc4ccccc45)n3)ccc2c1 ; c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1]

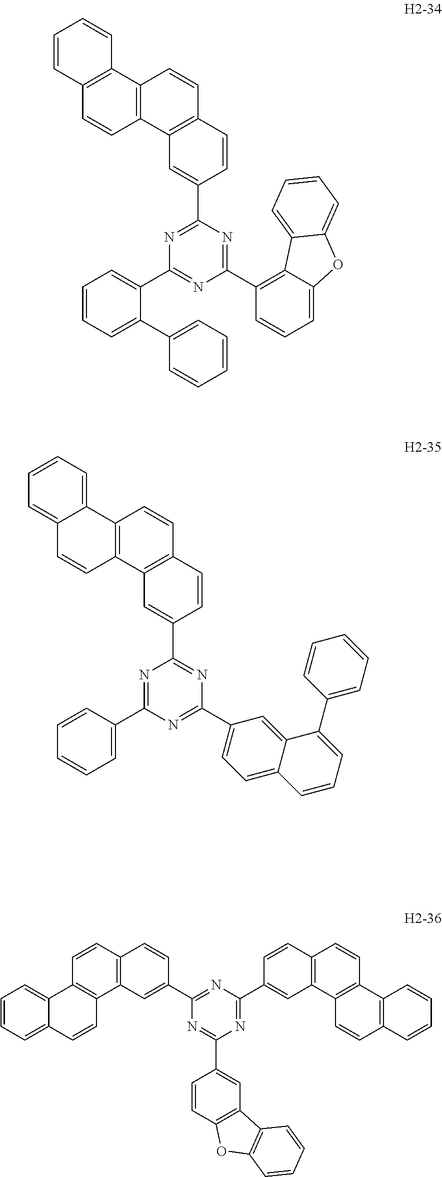 [SMILES: c1ccc(-c2ccccc2-c2nc(-c3ccc4ccc5c6ccccc6ccc5c4c3)nc(-c3cccc4oc5ccccc5c34)n2)cc1 ; c1ccc(-c2nc(-c3ccc4cccc(-c5ccccc5)c4c3)nc(-c3ccc4ccc5c6ccccc6ccc5c4c3)n2)cc1 ; c1ccc2c(c1)ccc1c3cc(-c4nc(-c5ccc6ccc7c8ccccc8ccc7c6c5)nc(-c5ccc6oc7ccccc7c6c5)n4)ccc3ccc21]

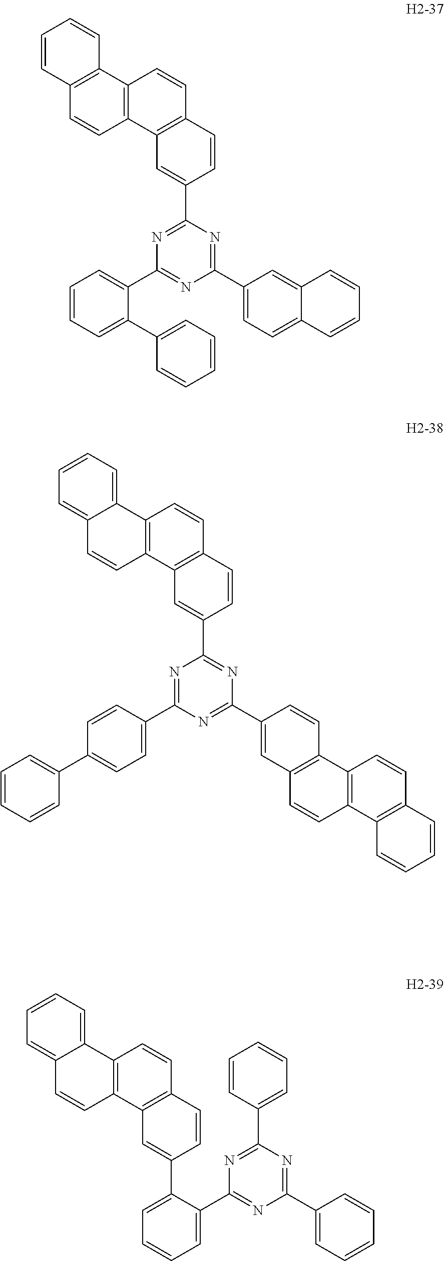 [SMILES: c1ccc(-c2ccccc2-c2nc(-c3ccc4ccccc4c3)nc(-c3ccc4ccc5c6ccccc6ccc5c4c3)n2)cc1 ; c1ccc(-c2ccc(-c3nc(-c4ccc5c(ccc6c7ccccc7ccc56)c4)nc(-c4ccc5ccc6c7ccccc7ccc6c5c4)n3)cc2)cc1 ; c1ccc(-c2nc(-c3ccccc3)nc(-c3ccccc3-c3ccc4ccc5c6ccccc6ccc5c4c3)n2)cc1]

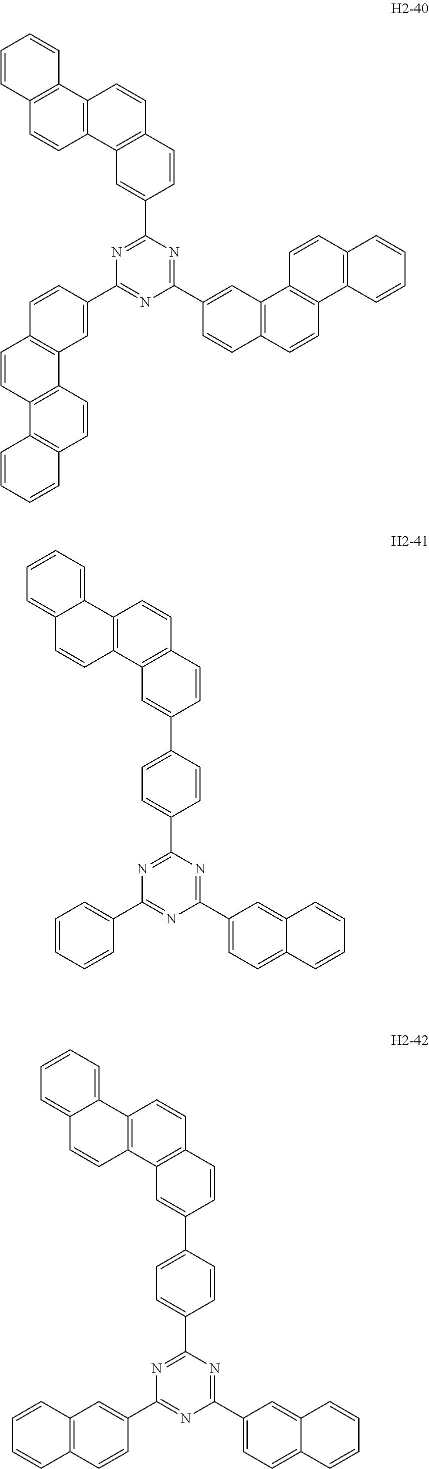 [SMILES: c1ccc2c(c1)ccc1c3cc(-c4nc(-c5ccc6ccc7c8ccccc8ccc7c6c5)nc(-c5ccc6ccc7c8ccccc8ccc7c6c5)n4)ccc3ccc21 ; c1ccc(-c2nc(-c3ccc(-c4ccc5ccc6c7ccccc7ccc6c5c4)cc3)nc(-c3ccc4ccccc4c3)n2)cc1 ; c1ccc2cc(-c3nc(-c4ccc(-c5ccc6ccc7c8ccccc8ccc7c6c5)cc4)nc(-c4ccc5ccccc5c4)n3)ccc2c1]

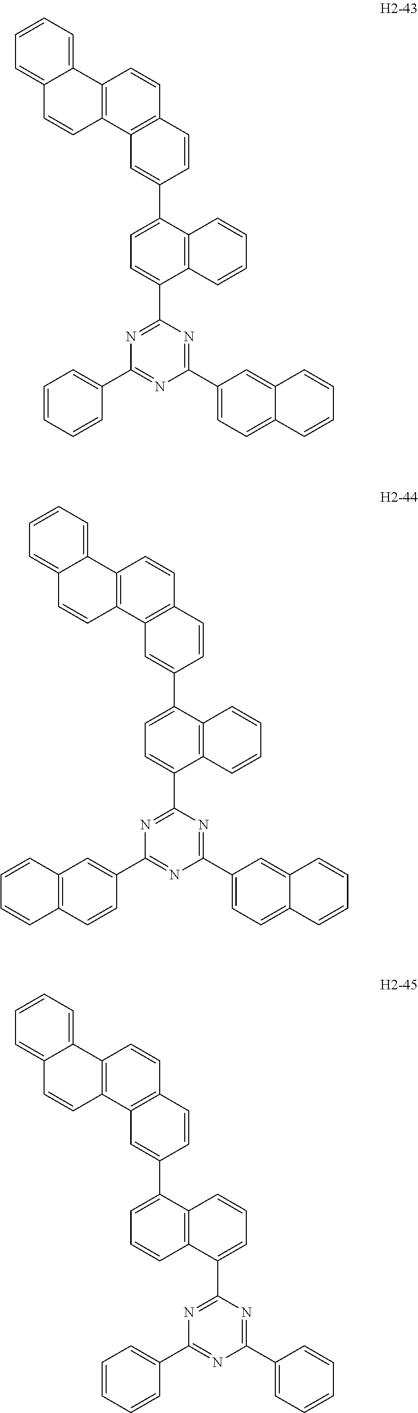 [SMILES: c1ccc(-c2nc(-c3ccc4ccccc4c3)nc(-c3ccc(-c4ccc5ccc6c7ccccc7ccc6c5c4)c4ccccc34)n2)cc1 ; c1ccc2cc(-c3nc(-c4ccc5ccccc5c4)nc(-c4ccc(-c5ccc6ccc7c8ccccc8ccc7c6c5)c5ccccc45)n3)ccc2c1 ; c1ccc(-c2nc(-c3ccccc3)nc(-c3cccc4c(-c5ccc6ccc7c8ccccc8ccc7c6c5)cccc34)n2)cc1]

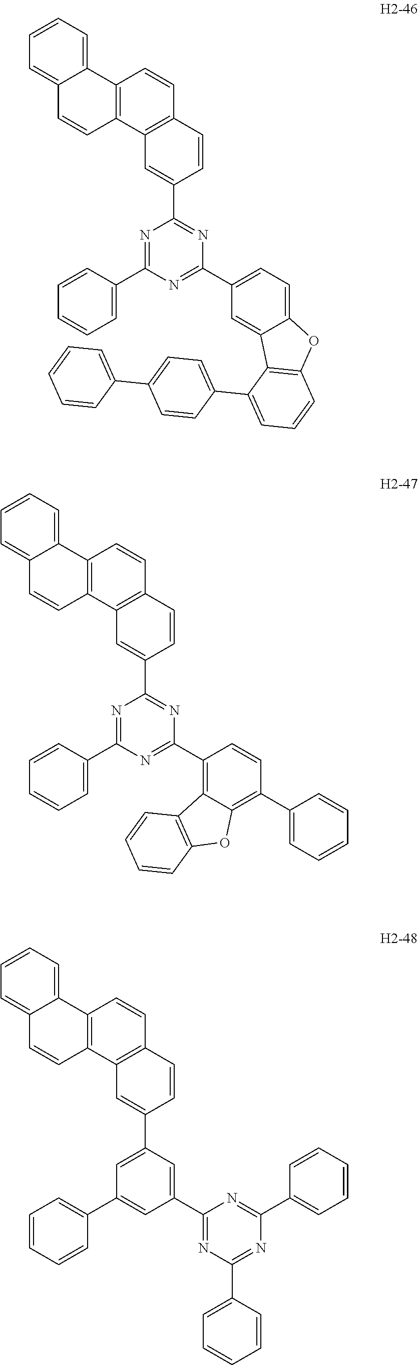 [SMILES: c1ccc(-c2ccc(-c3cccc4oc5ccc(-c6nc(-c7ccccc7)nc(-c7ccc8ccc9c%10ccccc%10ccc9c8c7)n6)cc5c34)cc2)cc1 ; c1ccc(-c2nc(-c3ccc4ccc5c6ccccc6ccc5c4c3)nc(-c3ccc(-c4ccccc4)c4oc5ccccc5c34)n2)cc1 ; c1ccc(-c2cc(-c3ccc4ccc5c6ccccc6ccc5c4c3)cc(-c3nc(-c4ccccc4)nc(-c4ccccc4)n3)c2)cc1]

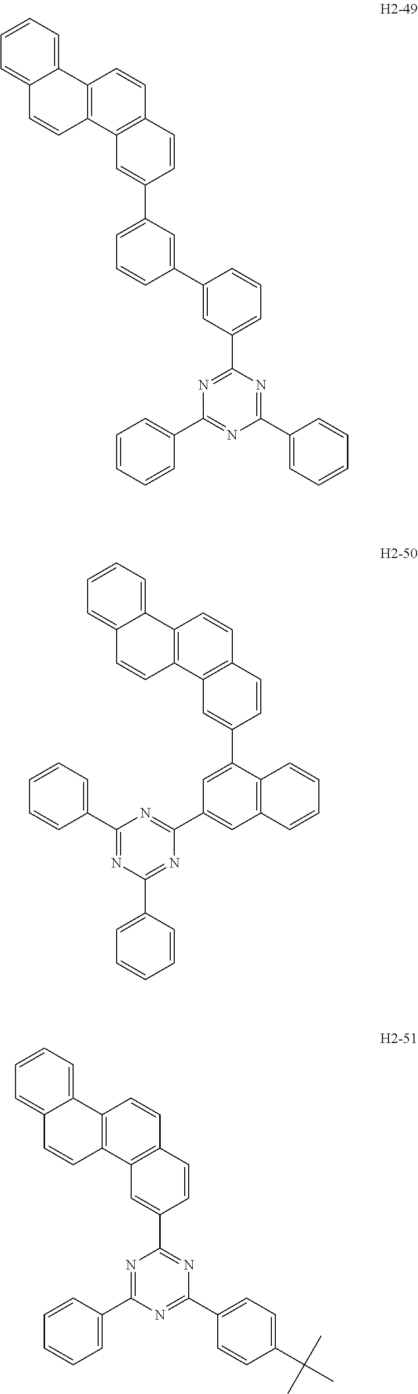 [SMILES: c1ccc(-c2nc(-c3ccccc3)nc(-c3cccc(-c4cccc(-c5ccc6ccc7c8ccccc8ccc7c6c5)c4)c3)n2)cc1 ; c1ccc(-c2nc(-c3ccccc3)nc(-c3cc(-c4ccc5ccc6c7ccccc7ccc6c5c4)c4ccccc4c3)n2)cc1 ; CC(C)(C)c1ccc(-c2nc(-c3ccccc3)nc(-c3ccc4ccc5c6ccccc6ccc5c4c3)n2)cc1]

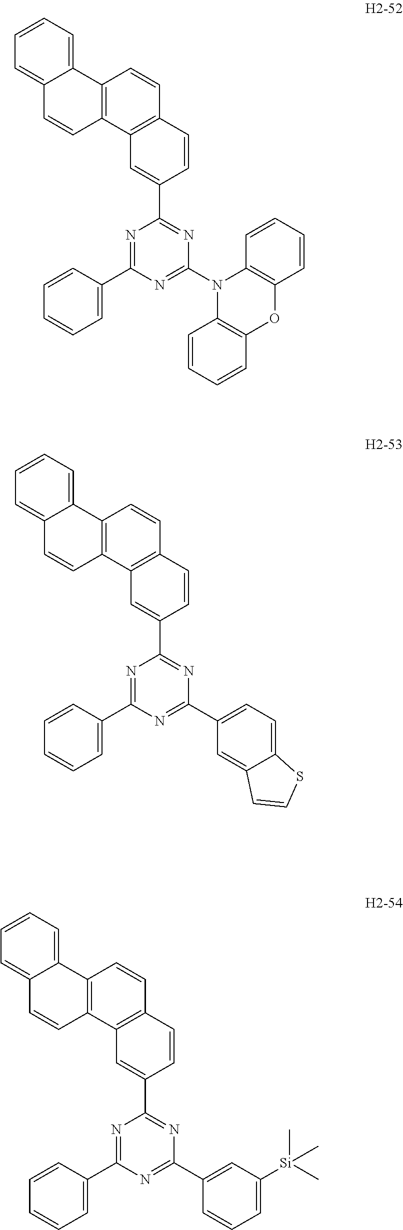 [SMILES: c1ccc(-c2nc(-c3ccc4ccc5c6ccccc6ccc5c4c3)nc(N3c4ccccc4Oc4ccccc43)n2)cc1 ; c1ccc(-c2nc(-c3ccc4sccc4c3)nc(-c3ccc4ccc5c6ccccc6ccc5c4c3)n2)cc1 ; C[Si](C)(C)c1cccc(-c2nc(-c3ccccc3)nc(-c3ccc4ccc5c6ccccc6ccc5c4c3)n2)c1]

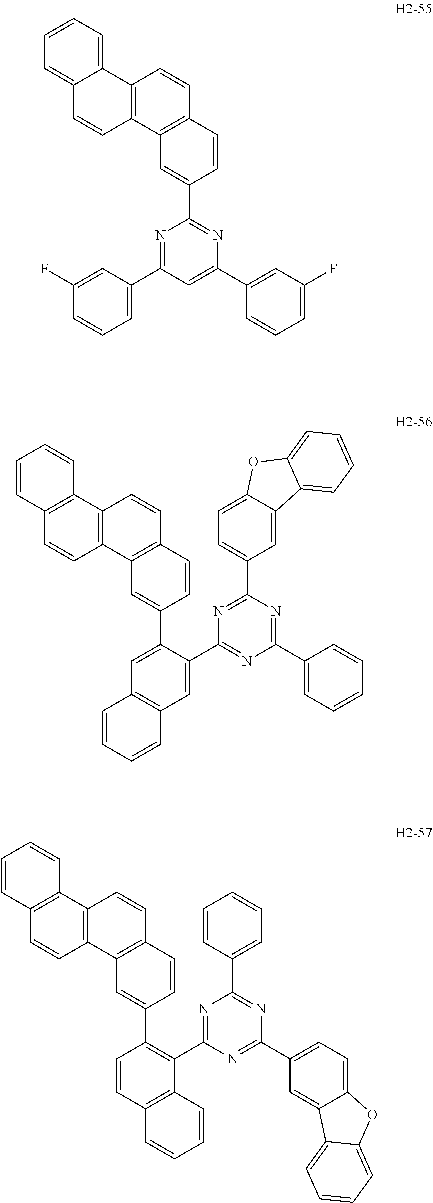 [SMILES: Fc1cccc(-c2cc(-c3cccc(F)c3)nc(-c3ccc4ccc5c6ccccc6ccc5c4c3)n2)c1 ; c1ccc(-c2nc(-c3ccc4oc5ccccc5c4c3)nc(-c3cc4ccccc4cc3-c3ccc4ccc5c6ccccc6ccc5c4c3)n2)cc1 ; c1ccc(-c2nc(-c3ccc4oc5ccccc5c4c3)nc(-c3c(-c4ccc5ccc6c7ccccc7ccc6c5c4)ccc4ccccc34)n2)cc1]

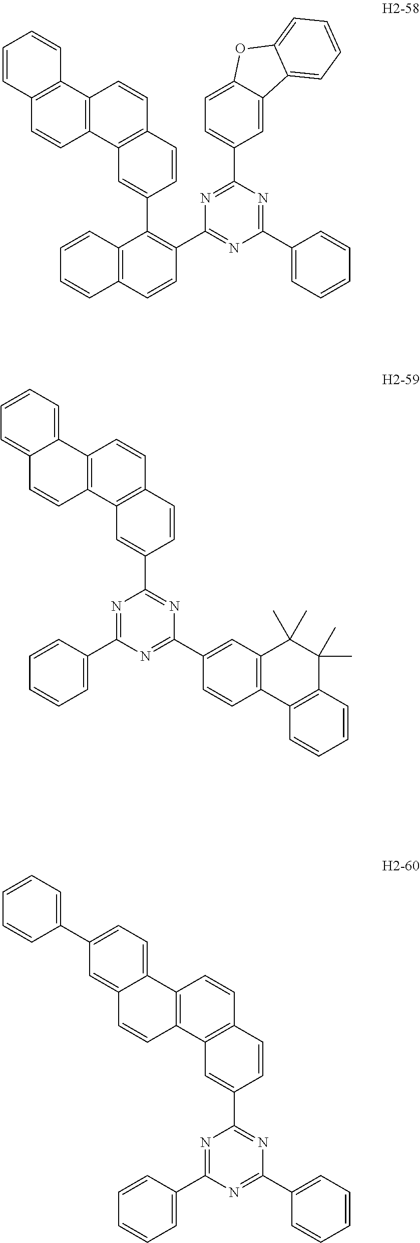 [SMILES: c1ccc(-c2nc(-c3ccc4oc5ccccc5c4c3)nc(-c3ccc4ccccc4c3-c3ccc4ccc5c6ccccc6ccc5c4c3)n2)cc1 ; CC1(C)c2ccccc2-c2ccc(-c3nc(-c4ccccc4)nc(-c4ccc5ccc6c7ccccc7ccc6c5c4)n3)cc2C1(C)C ; c1ccc(-c2ccc3c(ccc4c5cc(-c6nc(-c7ccccc7)nc(-c7ccccc7)n6)ccc5ccc34)c2)cc1]

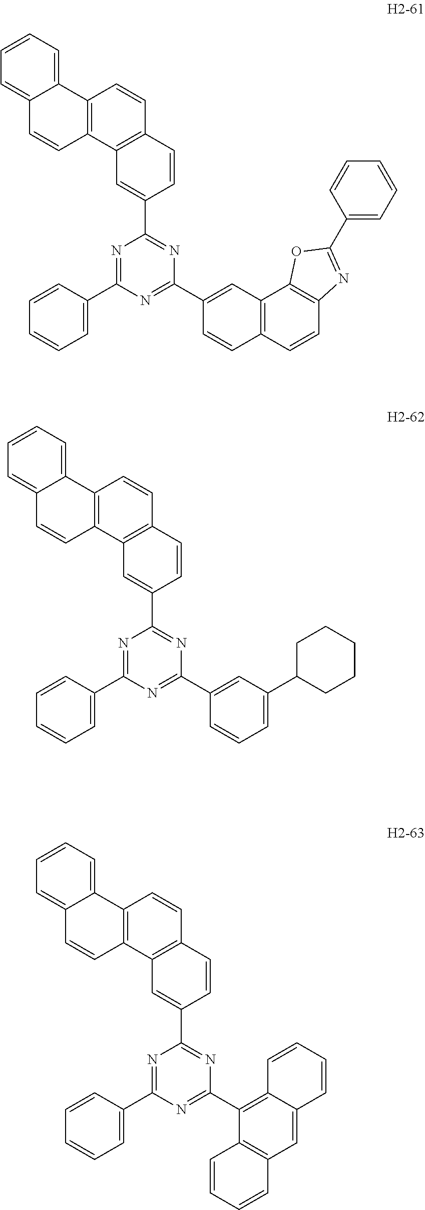 [SMILES: c1ccc(-c2nc(-c3ccc4ccc5c6ccccc6ccc5c4c3)nc(-c3ccc4ccc5nc(-c6ccccc6)oc5c4c3)n2)cc1 ; c1ccc(-c2nc(-c3cccc(C4CCCCC4)c3)nc(-c3ccc4ccc5c6ccccc6ccc5c4c3)n2)cc1 ; c1ccc(-c2nc(-c3ccc4ccc5c6ccccc6ccc5c4c3)nc(-c3c4ccccc4cc4ccccc34)n2)cc1]

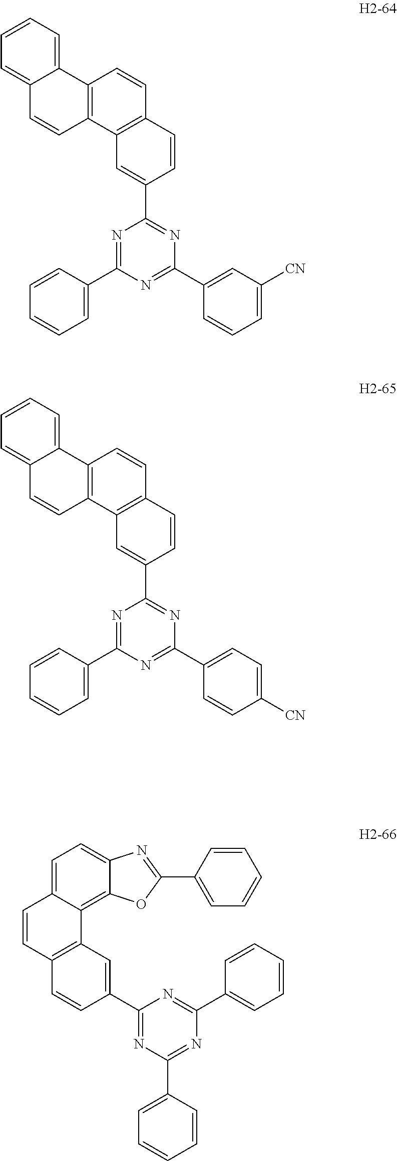 [SMILES: N#Cc1cccc(-c2nc(-c3ccccc3)nc(-c3ccc4ccc5c6ccccc6ccc5c4c3)n2)c1 ; N#Cc1ccc(-c2nc(-c3ccccc3)nc(-c3ccc4ccc5c6ccccc6ccc5c4c3)n2)cc1 ; c1ccc(-c2nc(-c3ccccc3)nc(-c3ccc4ccc5ccc6nc(-c7ccccc7)oc6c5c4c3)n2)cc1]

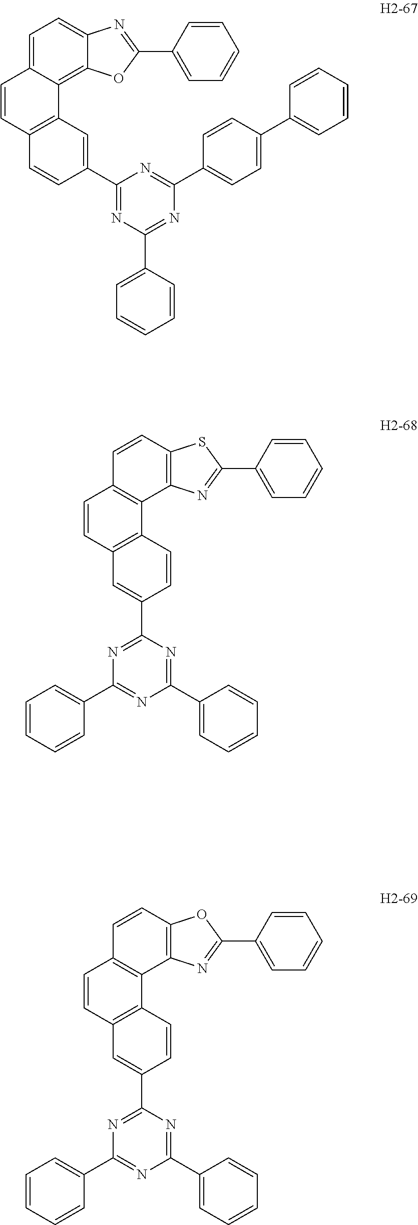 [SMILES: c1ccc(-c2ccc(-c3nc(-c4ccccc4)nc(-c4ccc5ccc6ccc7nc(-c8ccccc8)oc7c6c5c4)n3)cc2)cc1 ; c1ccc(-c2nc(-c3ccccc3)nc(-c3ccc4c(ccc5ccc6sc(-c7ccccc7)nc6c54)c3)n2)cc1 ; c1ccc(-c2nc(-c3ccccc3)nc(-c3ccc4c(ccc5ccc6oc(-c7ccccc7)nc6c54)c3)n2)cc1]

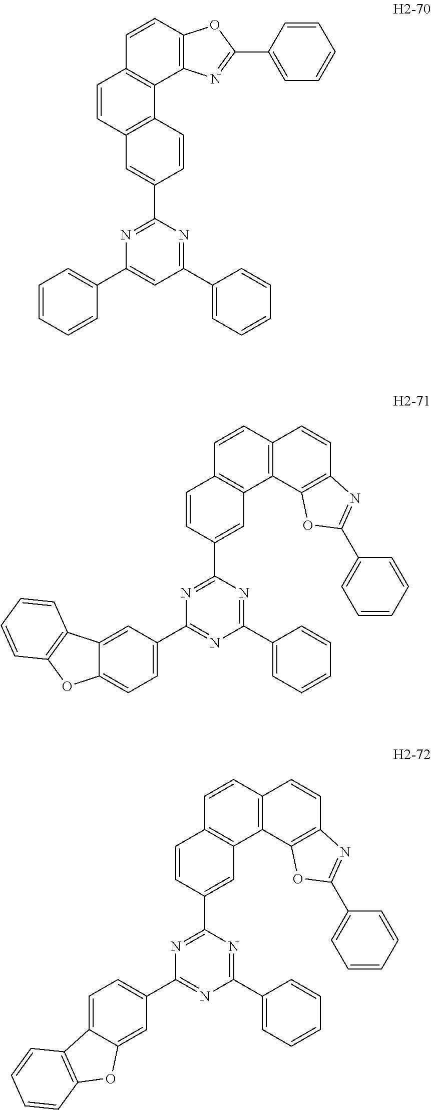 [SMILES: c1ccc(-c2cc(-c3ccccc3)nc(-c3ccc4c(ccc5ccc6oc(-c7ccccc7)nc6c54)c3)n2)cc1 ; c1ccc(-c2nc(-c3ccc4oc5ccccc5c4c3)nc(-c3ccc4ccc5ccc6nc(-c7ccccc7)oc6c5c4c3)n2)cc1 ; c1ccc(-c2nc(-c3ccc4c(c3)oc3ccccc34)nc(-c3ccc4ccc5ccc6nc(-c7ccccc7)oc6c5c4c3)n2)cc1]

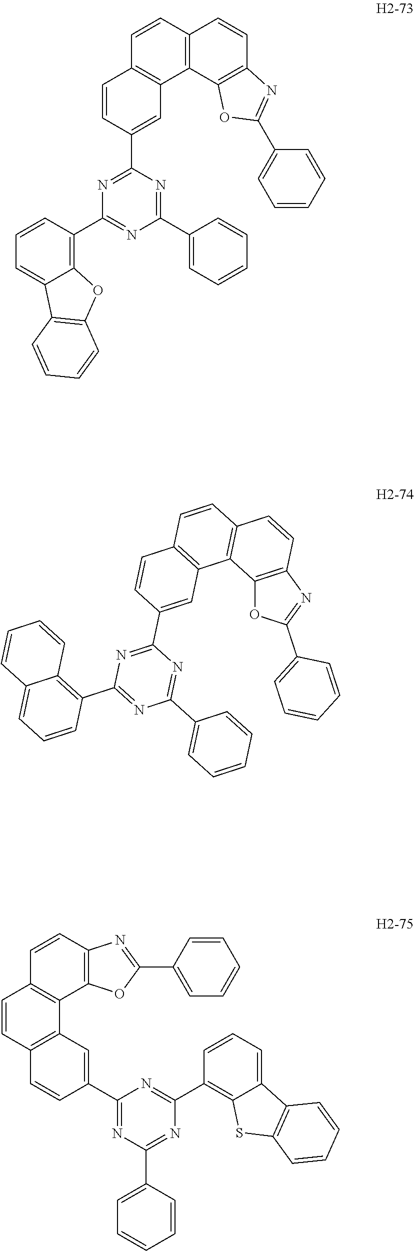 [SMILES: c1ccc(-c2nc(-c3ccc4ccc5ccc6nc(-c7ccccc7)oc6c5c4c3)nc(-c3cccc4c3oc3ccccc34)n2)cc1 ; c1ccc(-c2nc(-c3ccc4ccc5ccc6nc(-c7ccccc7)oc6c5c4c3)nc(-c3cccc4ccccc34)n2)cc1 ; c1ccc(-c2nc(-c3ccc4ccc5ccc6nc(-c7ccccc7)oc6c5c4c3)nc(-c3cccc4c3sc3ccccc34)n2)cc1]

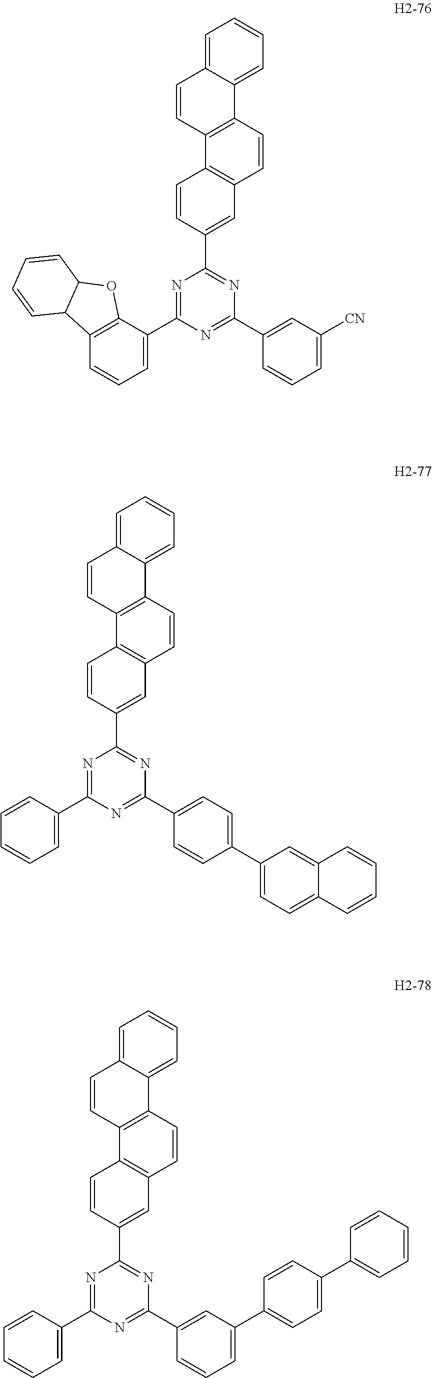 [SMILES: N#Cc1cccc(-c2nc(-c3ccc4c(ccc5c6ccccc6ccc45)c3)nc(-c3cccc4c3OC3C=CC=CC43)n2)c1 ; c1ccc(-c2nc(-c3ccc(-c4ccc5ccccc5c4)cc3)nc(-c3ccc4c(ccc5c6ccccc6ccc45)c3)n2)cc1 ; c1ccc(-c2ccc(-c3cccc(-c4nc(-c5ccccc5)nc(-c5ccc6c(ccc7c8ccccc8ccc67)c5)n4)c3)cc2)cc1]

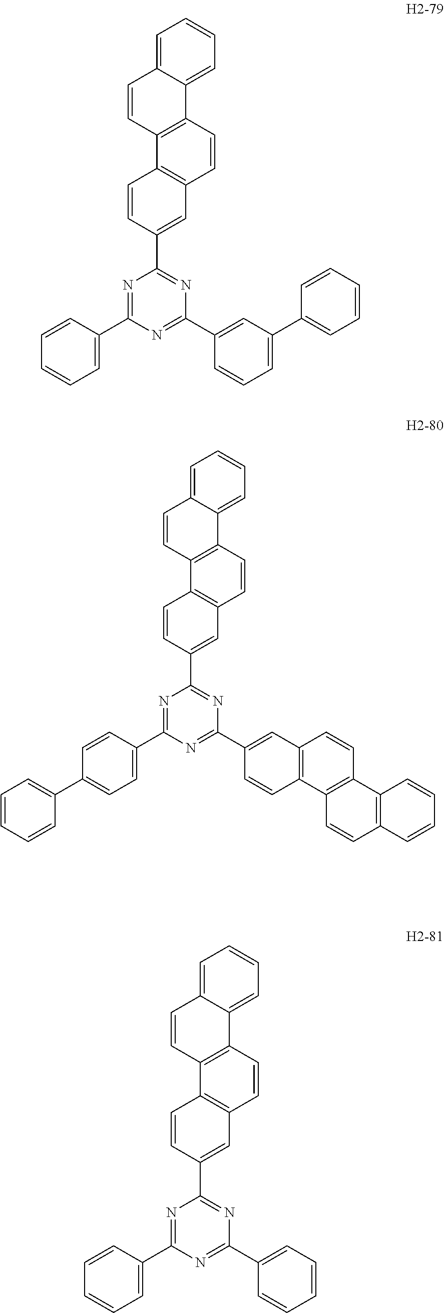 [SMILES: c1ccc(-c2cccc(-c3nc(-c4ccccc4)nc(-c4ccc5c(ccc6c7ccccc7ccc56)c4)n3)c2)cc1 ; c1ccc(-c2ccc(-c3nc(-c4ccc5c(ccc6c7ccccc7ccc56)c4)nc(-c4ccc5c(ccc6c7ccccc7ccc56)c4)n3)cc2)cc1 ; c1ccc(-c2nc(-c3ccccc3)nc(-c3ccc4c(ccc5c6ccccc6ccc45)c3)n2)cc1]

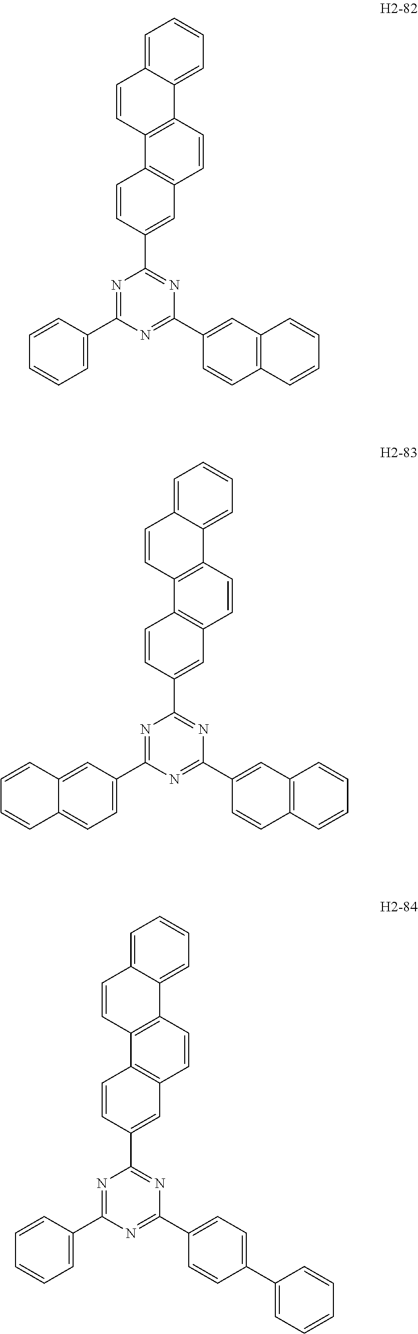 [SMILES: c1ccc(-c2nc(-c3ccc4ccccc4c3)nc(-c3ccc4c(ccc5c6ccccc6ccc45)c3)n2)cc1 ; c1ccc2cc(-c3nc(-c4ccc5ccccc5c4)nc(-c4ccc5c(ccc6c7ccccc7ccc56)c4)n3)ccc2c1 ; c1ccc(-c2ccc(-c3nc(-c4ccccc4)nc(-c4ccc5c(ccc6c7ccccc7ccc56)c4)n3)cc2)cc1]

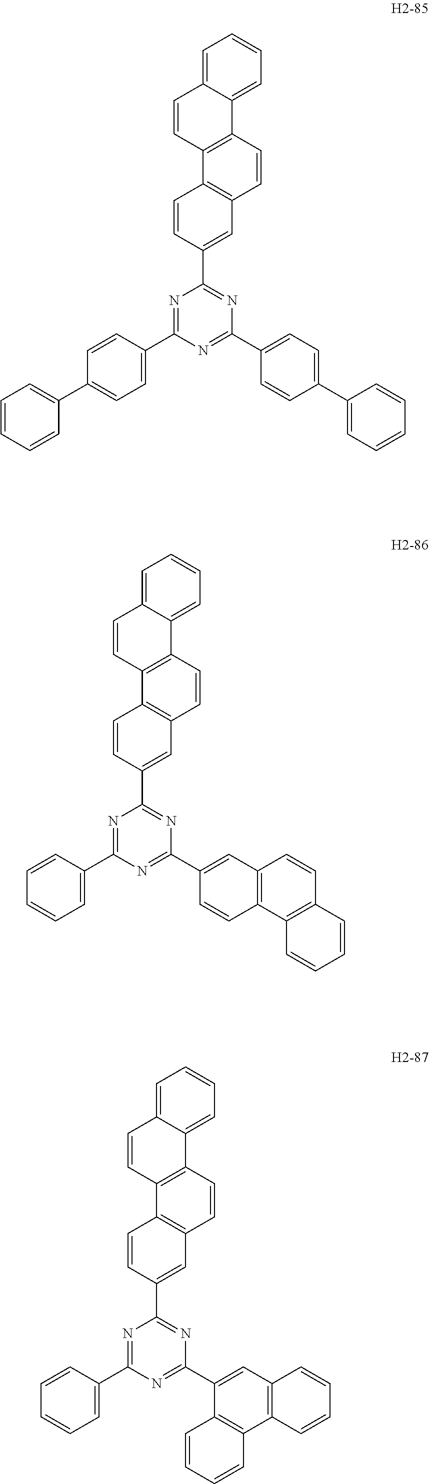 [SMILES: c1ccc(-c2ccc(-c3nc(-c4ccc(-c5ccccc5)cc4)nc(-c4ccc5c(ccc6c7ccccc7ccc56)c4)n3)cc2)cc1 ; c1ccc(-c2nc(-c3ccc4c(ccc5ccccc54)c3)nc(-c3ccc4c(ccc5c6ccccc6ccc45)c3)n2)cc1 ; c1ccc(-c2nc(-c3ccc4c(ccc5c6ccccc6ccc45)c3)nc(-c3cc4ccccc4c4ccccc34)n2)cc1]

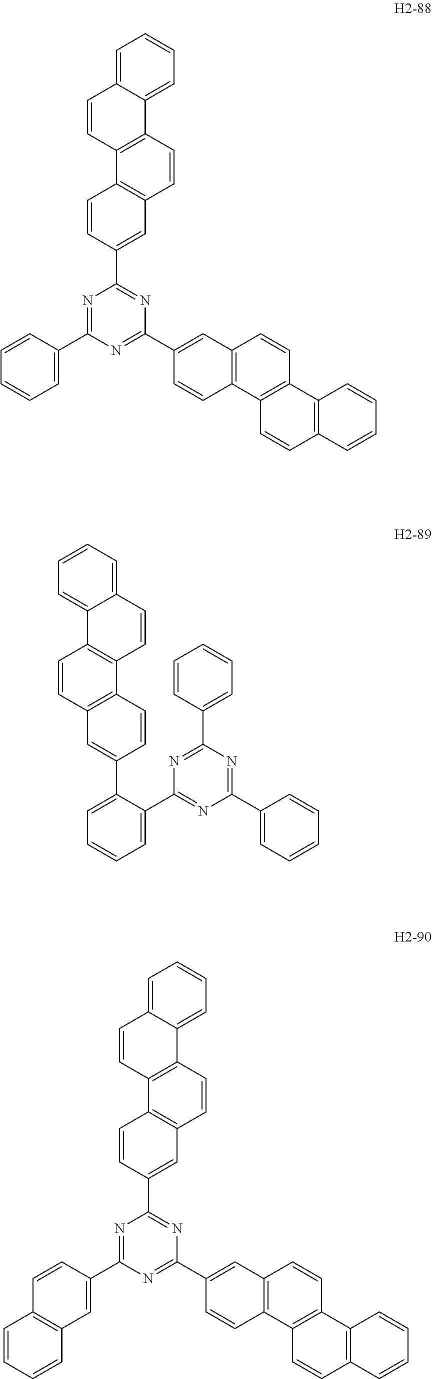 [SMILES: c1ccc(-c2nc(-c3ccc4c(ccc5c6ccccc6ccc45)c3)nc(-c3ccc4c(ccc5c6ccccc6ccc45)c3)n2)cc1 ; c1ccc(-c2nc(-c3ccccc3)nc(-c3ccccc3-c3ccc4c(ccc5c6ccccc6ccc45)c3)n2)cc1 ; c1ccc2cc(-c3nc(-c4ccc5c(ccc6c7ccccc7ccc56)c4)nc(-c4ccc5c(ccc6c7ccccc7ccc56)c4)n3)ccc2c1]

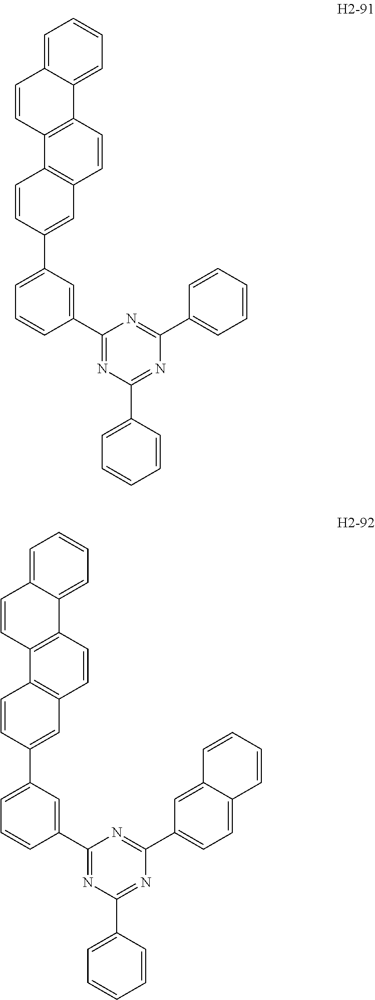 [SMILES: c1ccc(-c2nc(-c3ccccc3)nc(-c3cccc(-c4ccc5c(ccc6c7ccccc7ccc56)c4)c3)n2)cc1 ; c1ccc(-c2nc(-c3cccc(-c4ccc5c(ccc6c7ccccc7ccc56)c4)c3)nc(-c3ccc4ccccc4c3)n2)cc1]

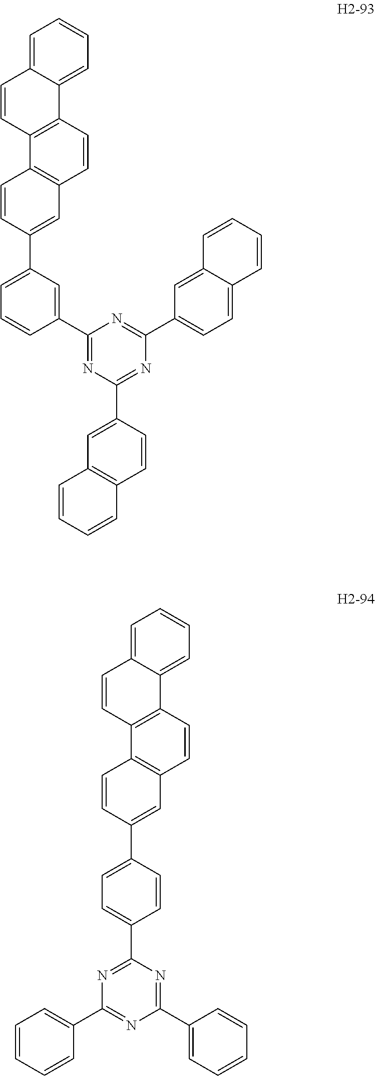 [SMILES: c1cc(-c2ccc3c(ccc4c5ccccc5ccc34)c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5c(ccc6c7ccccc7ccc56)c4)cc3)n2)cc1]

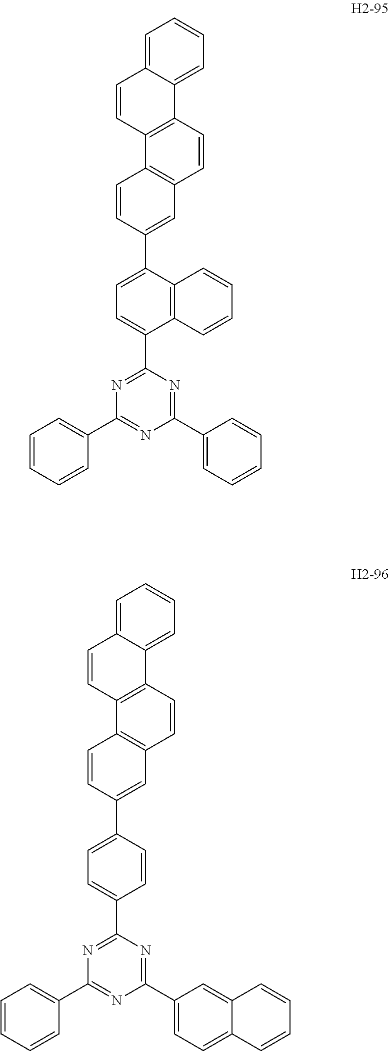 [SMILES: c1ccc(-c2nc(-c3ccccc3)nc(-c3ccc(-c4ccc5c(ccc6c7ccccc7ccc56)c4)c4ccccc34)n2)cc1 ; c1ccc(-c2nc(-c3ccc(-c4ccc5c(ccc6c7ccccc7ccc56)c4)cc3)nc(-c3ccc4ccccc4c3)n2)cc1]

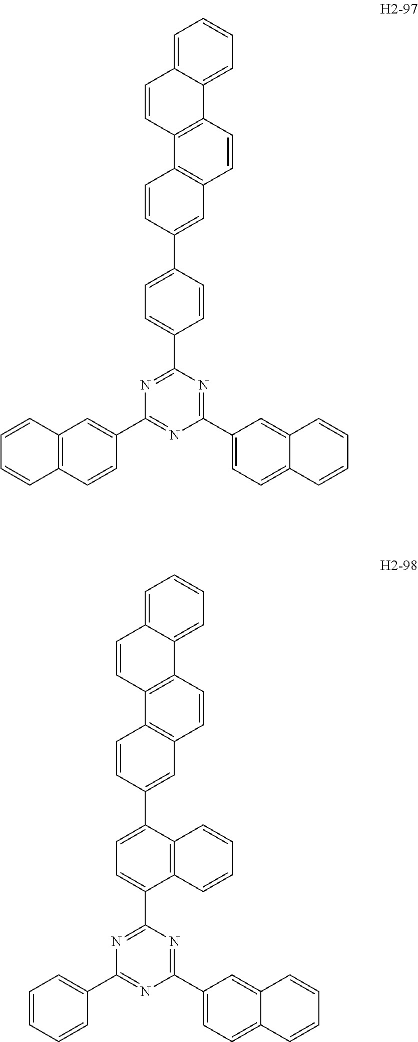 [SMILES: c1ccc2cc(-c3nc(-c4ccc(-c5ccc6c(ccc7c8ccccc8ccc67)c5)cc4)nc(-c4ccc5ccccc5c4)n3)ccc2c1 ; c1ccc(-c2nc(-c3ccc4ccccc4c3)nc(-c3ccc(-c4ccc5c(ccc6c7ccccc7ccc56)c4)c4ccccc34)n2)cc1]

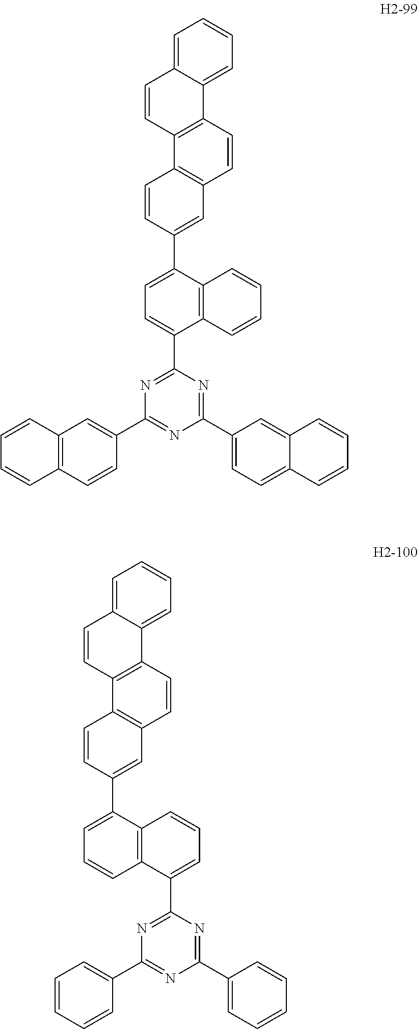 [SMILES: c1ccc2cc(-c3nc(-c4ccc5ccccc5c4)nc(-c4ccc(-c5ccc6c(ccc7c8ccccc8ccc67)c5)c5ccccc45)n3)ccc2c1 ; c1ccc(-c2nc(-c3ccccc3)nc(-c3cccc4c(-c5ccc6c(ccc7c8ccccc8ccc67)c5)cccc34)n2)cc1]

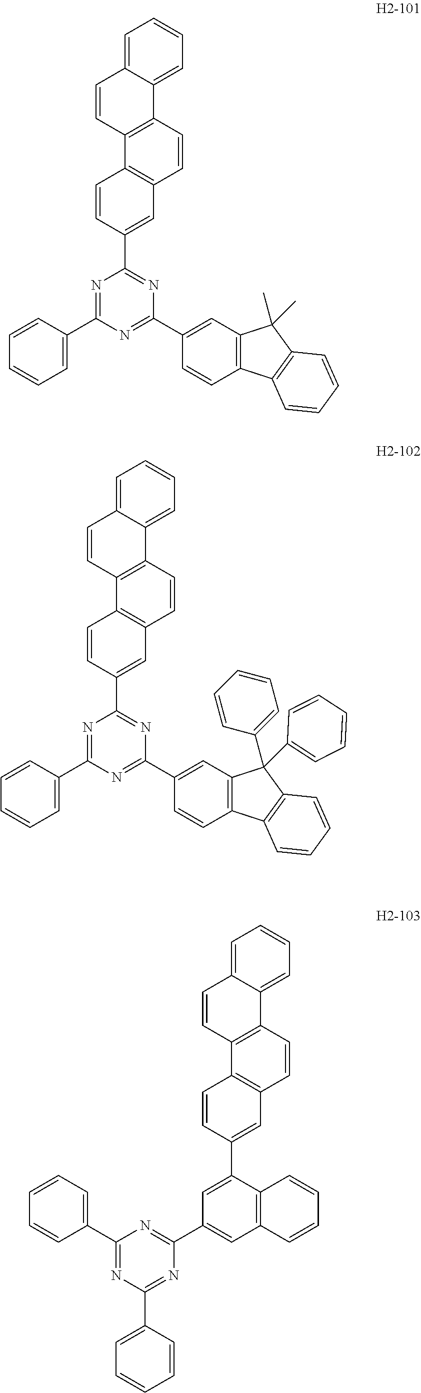 [SMILES: CC1(C)c2ccccc2-c2ccc(-c3nc(-c4ccccc4)nc(-c4ccc5c(ccc6c7ccccc7ccc56)c4)n3)cc21 ; c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1 ; c1ccc(-c2nc(-c3ccccc3)nc(-c3cc(-c4ccc5c(ccc6c7ccccc7ccc56)c4)c4ccccc4c3)n2)cc1]

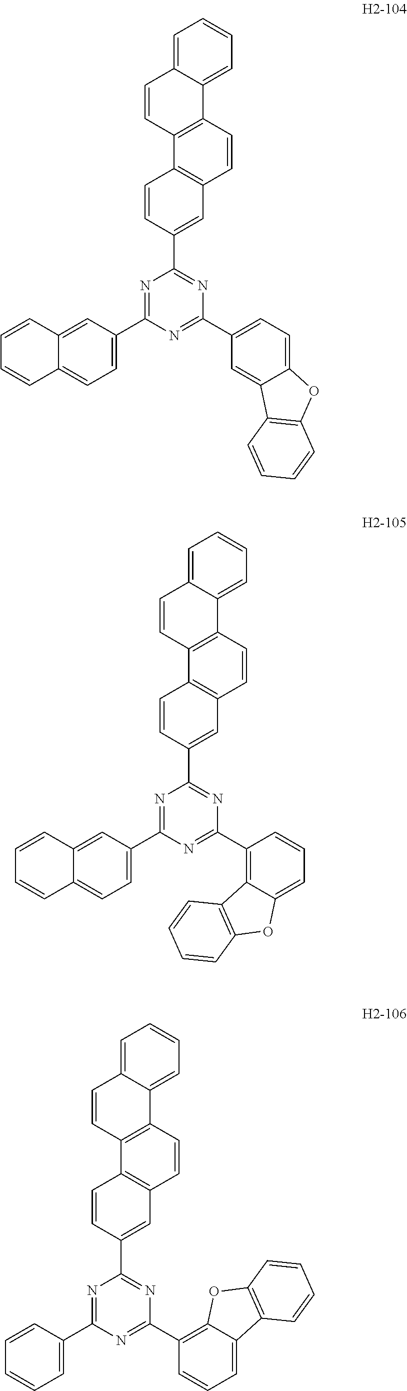 [SMILES: c1ccc2cc(-c3nc(-c4ccc5c(ccc6c7ccccc7ccc56)c4)nc(-c4ccc5oc6ccccc6c5c4)n3)ccc2c1 ; c1ccc2cc(-c3nc(-c4ccc5c(ccc6c7ccccc7ccc56)c4)nc(-c4cccc5oc6ccccc6c45)n3)ccc2c1 ; c1ccc(-c2nc(-c3ccc4c(ccc5c6ccccc6ccc45)c3)nc(-c3cccc4c3oc3ccccc34)n2)cc1]

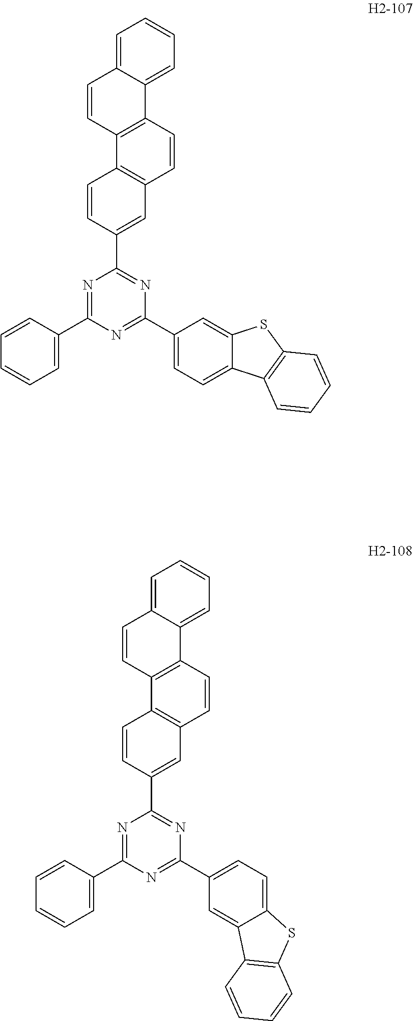 [SMILES: c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1]

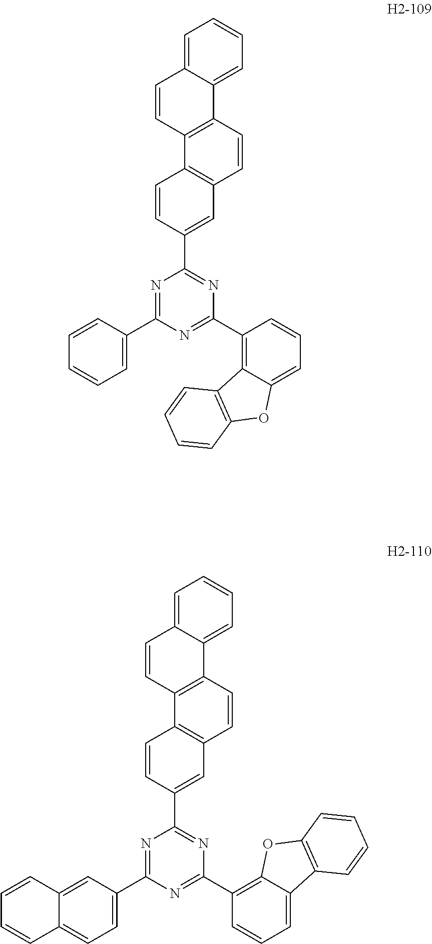 [SMILES: c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1]

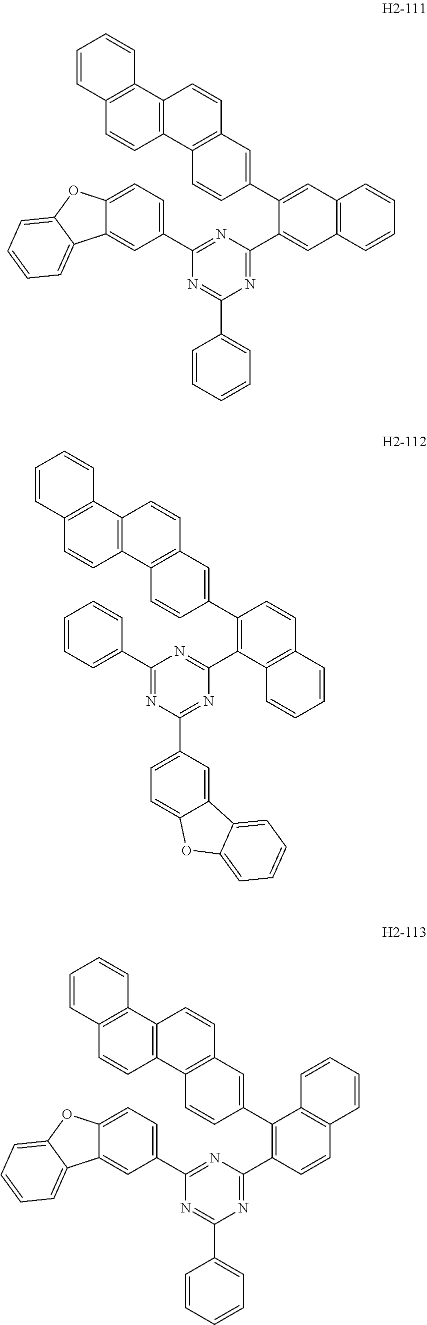 [SMILES: c1ccc(-c2nc(-c3ccc4oc5ccccc5c4c3)nc(-c3cc4ccccc4cc3-c3ccc4c(ccc5c6ccccc6ccc45)c3)n2)cc1 ; c1ccc(-c2nc(-c3ccc4oc5ccccc5c4c3)nc(-c3c(-c4ccc5c(ccc6c7ccccc7ccc56)c4)ccc4ccccc34)n2)cc1 ; c1ccc(-c2nc(-c3ccc4oc5ccccc5c4c3)nc(-c3ccc4ccccc4c3-c3ccc4c(ccc5c6ccccc6ccc45)c3)n2)cc1]

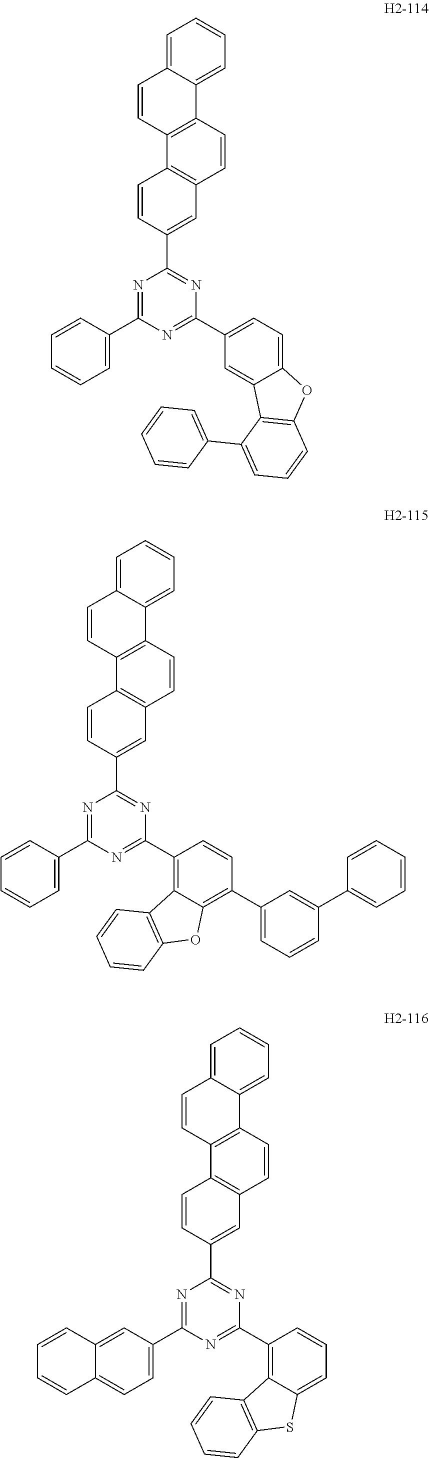 [SMILES: c1ccc(-c2nc(-c3ccc4c(ccc5c6ccccc6ccc45)c3)nc(-c3ccc4oc5cccc(-c6ccccc6)c5c4c3)n2)cc1 ; c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4cccc5sc6ccccc6c45)n3)ccc2c1]

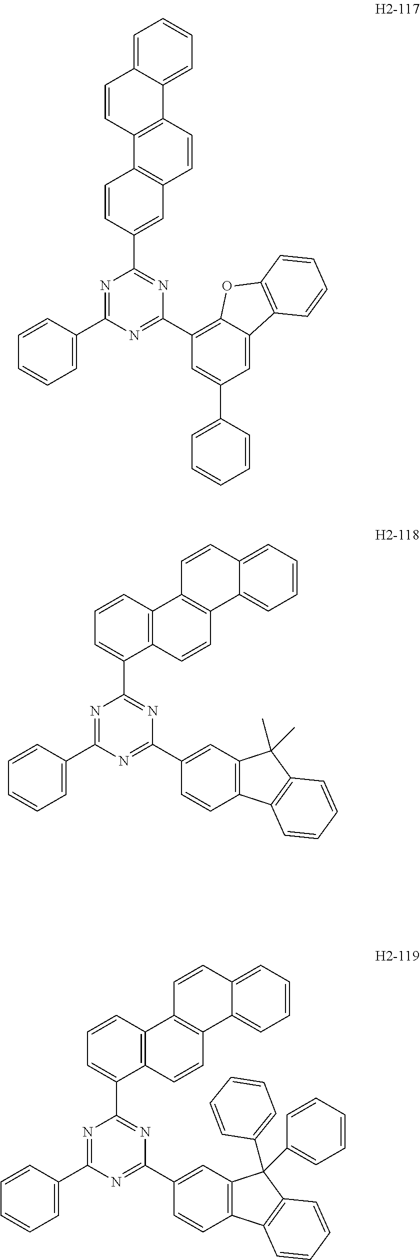 [SMILES: c1ccc(-c2cc(-c3nc(-c4ccccc4)nc(-c4ccc5c(ccc6c7ccccc7ccc56)c4)n3)c3oc4ccccc4c3c2)cc1 ; CC1(C)c2ccccc2-c2ccc(-c3nc(-c4ccccc4)nc(-c4cccc5c4ccc4c6ccccc6ccc54)n3)cc21 ; c1ccc(-c2nc(-c3ccc4c(c3)C(c3ccccc3)(c3ccccc3)c3ccccc3-4)nc(-c3cccc4c3ccc3c5ccccc5ccc43)n2)cc1]

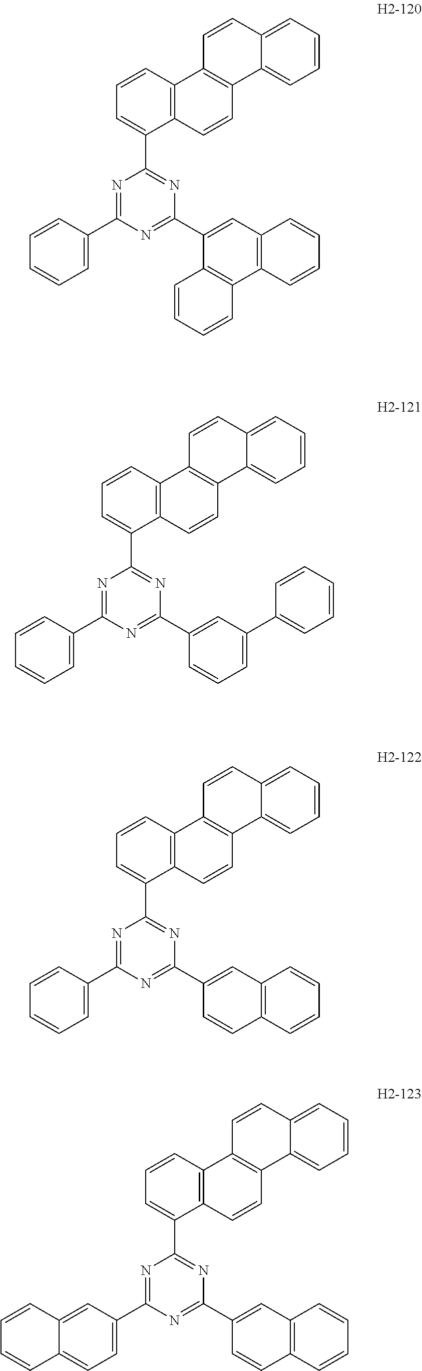 [SMILES: c1ccc(-c2nc(-c3cc4ccccc4c4ccccc34)nc(-c3cccc4c3ccc3c5ccccc5ccc43)n2)cc1 ; c1ccc(-c2cccc(-c3nc(-c4ccccc4)nc(-c4cccc5c4ccc4c6ccccc6ccc54)n3)c2)cc1 ; c1ccc(-c2nc(-c3ccc4ccccc4c3)nc(-c3cccc4c3ccc3c5ccccc5ccc43)n2)cc1 ; c1ccc2cc(-c3nc(-c4ccc5ccccc5c4)nc(-c4cccc5c4ccc4c6ccccc6ccc54)n3)ccc2c1]

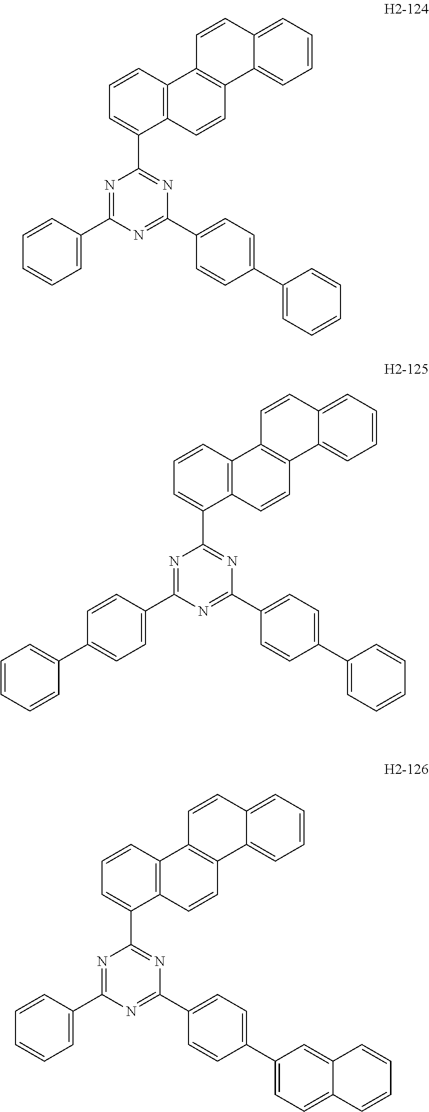 [SMILES: c1ccc(-c2ccc(-c3nc(-c4ccccc4)nc(-c4cccc5c4ccc4c6ccccc6ccc54)n3)cc2)cc1 ; c1ccc(-c2ccc(-c3nc(-c4ccc(-c5ccccc5)cc4)nc(-c4cccc5c4ccc4c6ccccc6ccc54)n3)cc2)cc1 ; c1ccc(-c2nc(-c3ccc(-c4ccc5ccccc5c4)cc3)nc(-c3cccc4c3ccc3c5ccccc5ccc43)n2)cc1]

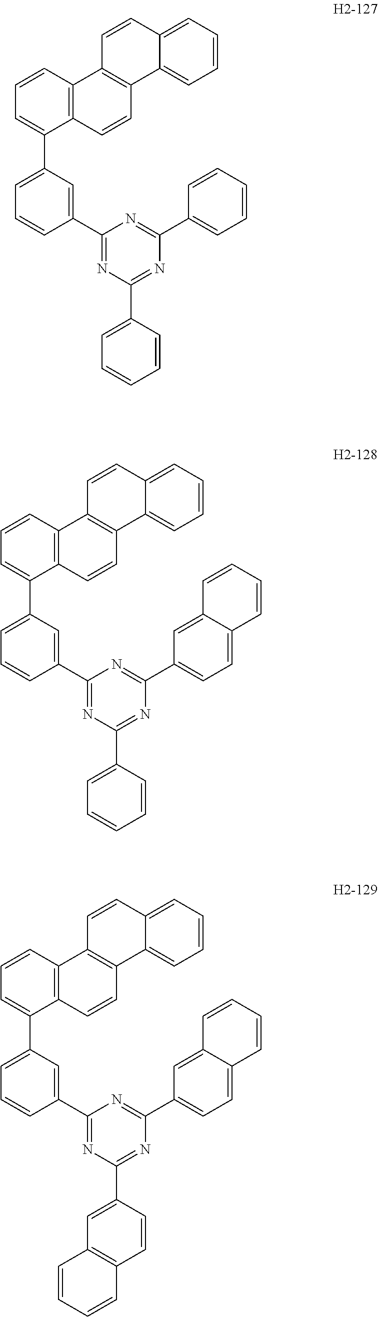 [SMILES: c1ccc(-c2nc(-c3ccccc3)nc(-c3cccc(-c4cccc5c4ccc4c6ccccc6ccc54)c3)n2)cc1 ; c1ccc(-c2nc(-c3cccc(-c4cccc5c4ccc4c6ccccc6ccc54)c3)nc(-c3ccc4ccccc4c3)n2)cc1 ; c1cc(-c2nc(-c3ccc4ccccc4c3)nc(-c3ccc4ccccc4c3)n2)cc(-c2cccc3c2ccc2c4ccccc4ccc32)c1]

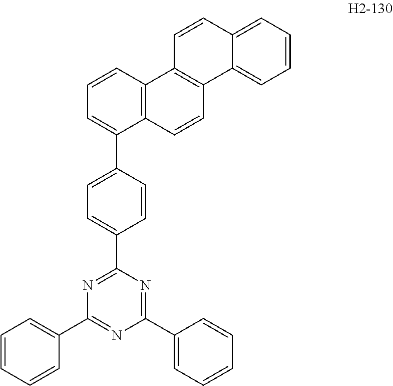 [SMILES: c1ccc(-c2nc(-c3ccccc3)nc(-c3ccc(-c4cccc5c4ccc4c6ccccc6ccc54)cc3)n2)cc1]

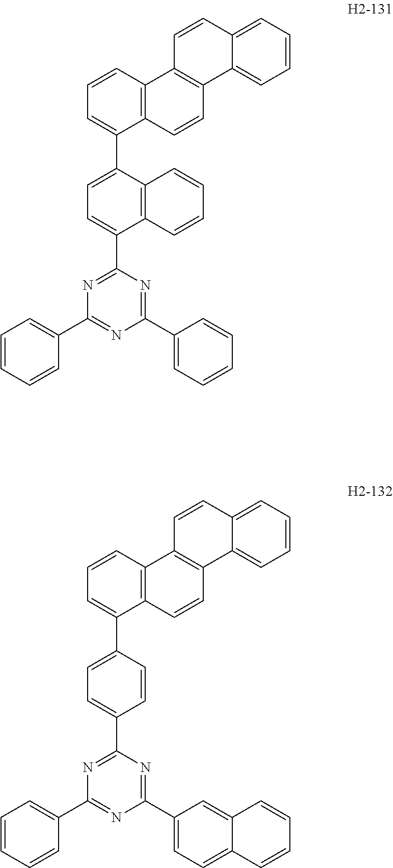 [SMILES: c1ccc(-c2nc(-c3ccccc3)nc(-c3ccc(-c4cccc5c4ccc4c6ccccc6ccc54)c4ccccc34)n2)cc1 ; c1ccc(-c2nc(-c3ccc(-c4cccc5c4ccc4c6ccccc6ccc54)cc3)nc(-c3ccc4ccccc4c3)n2)cc1]

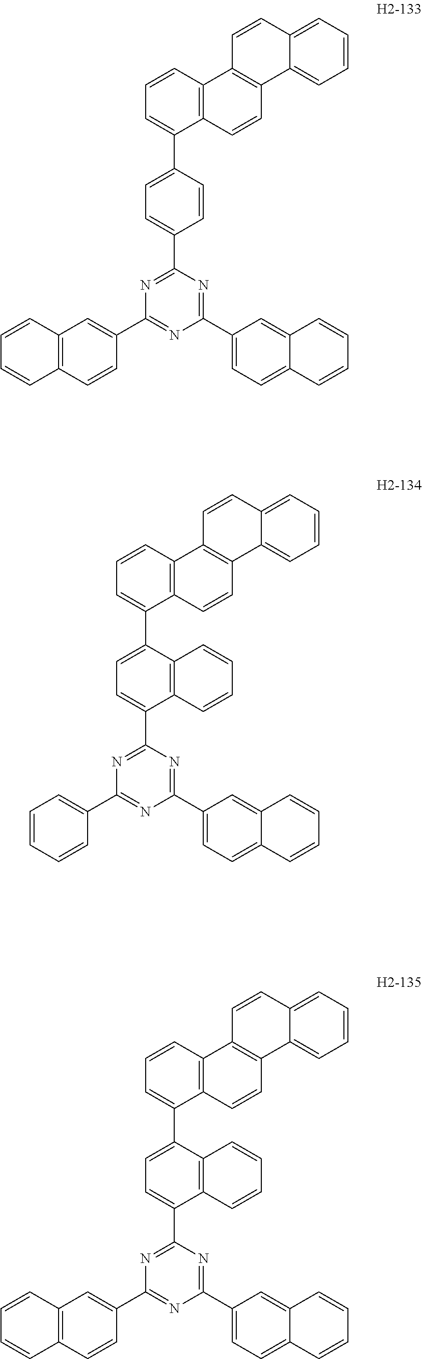 [SMILES: c1ccc2cc(-c3nc(-c4ccc(-c5cccc6c5ccc5c7ccccc7ccc65)cc4)nc(-c4ccc5ccccc5c4)n3)ccc2c1 ; c1ccc(-c2nc(-c3ccc4ccccc4c3)nc(-c3ccc(-c4cccc5c4ccc4c6ccccc6ccc54)c4ccccc34)n2)cc1 ; c1ccc2cc(-c3nc(-c4ccc5ccccc5c4)nc(-c4ccc(-c5cccc6c5ccc5c7ccccc7ccc65)c5ccccc45)n3)ccc2c1]

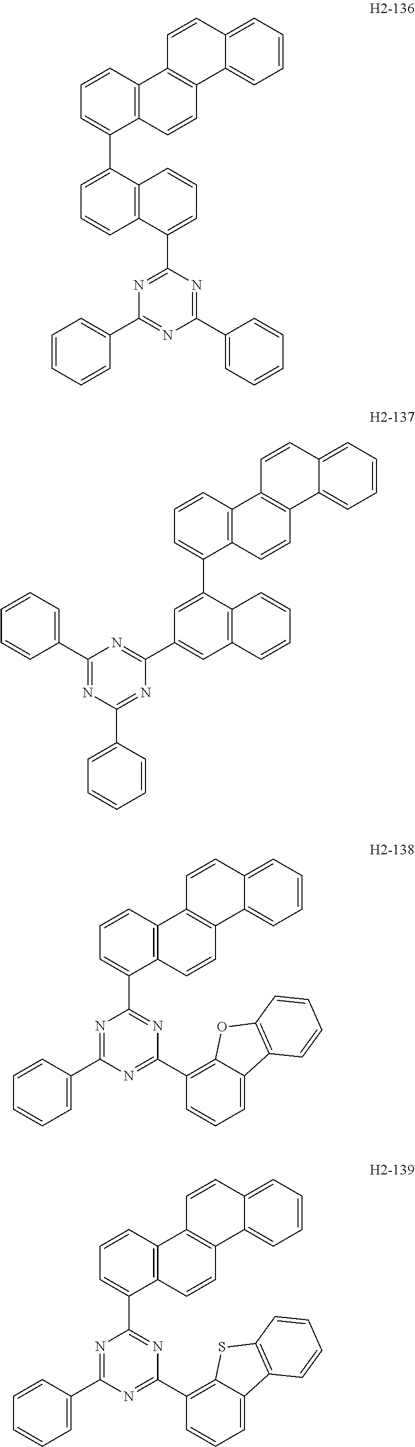 [SMILES: c1ccc(-c2nc(-c3ccccc3)nc(-c3cccc4c(-c5cccc6c5ccc5c7ccccc7ccc65)cccc34)n2)cc1 ; c1ccc(-c2nc(-c3ccccc3)nc(-c3cc(-c4cccc5c4ccc4c6ccccc6ccc54)c4ccccc4c3)n2)cc1 ; c1ccc(-c2nc(-c3cccc4c3ccc3c5ccccc5ccc43)nc(-c3cccc4c3oc3ccccc34)n2)cc1 ; c1ccc(-c2nc(-c3cccc4c3ccc3c5ccccc5ccc43)nc(-c3cccc4c3sc3ccccc34)n2)cc1]

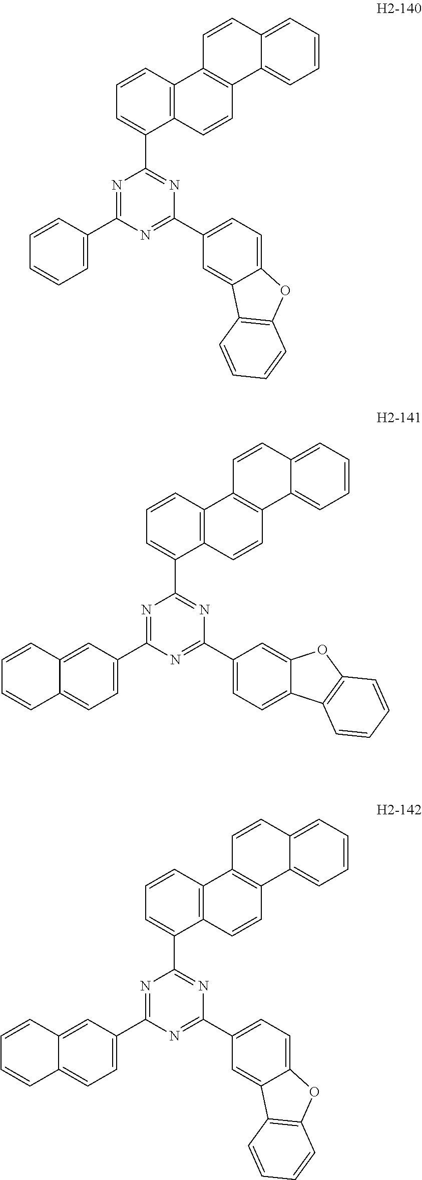 [SMILES: c1ccc(-c2nc(-c3ccc4oc5ccccc5c4c3)nc(-c3cccc4c3ccc3c5ccccc5ccc43)n2)cc1 ; c1ccc2cc(-c3nc(-c4ccc5c(c4)oc4ccccc45)nc(-c4cccc5c4ccc4c6ccccc6ccc54)n3)ccc2c1 ; c1ccc2cc(-c3nc(-c4ccc5oc6ccccc6c5c4)nc(-c4cccc5c4ccc4c6ccccc6ccc54)n3)ccc2c1]

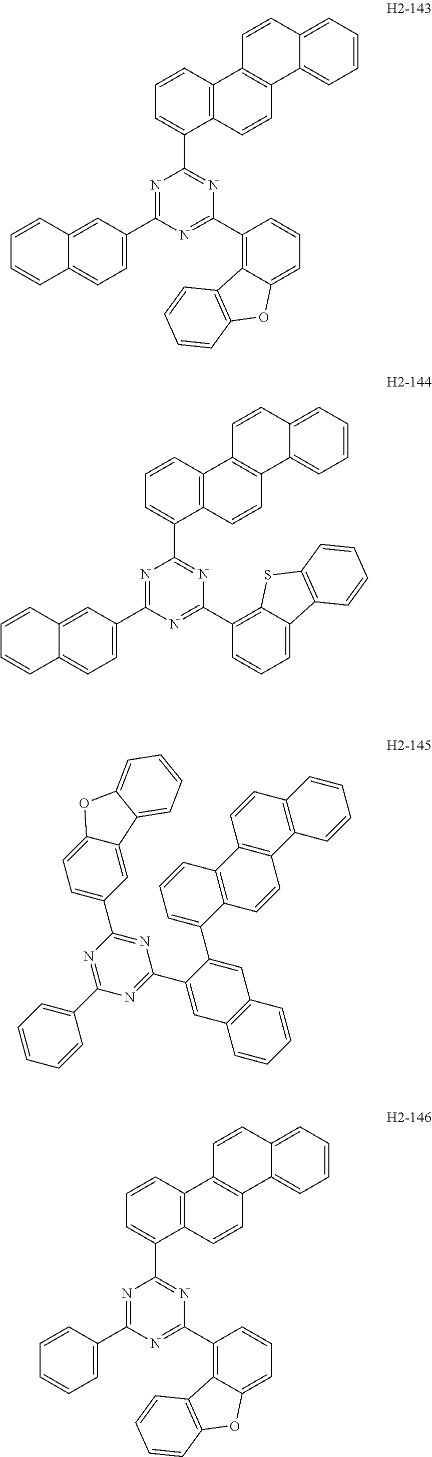 [SMILES: c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1 ; c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4oc5ccccc5c34)n2)cc1]

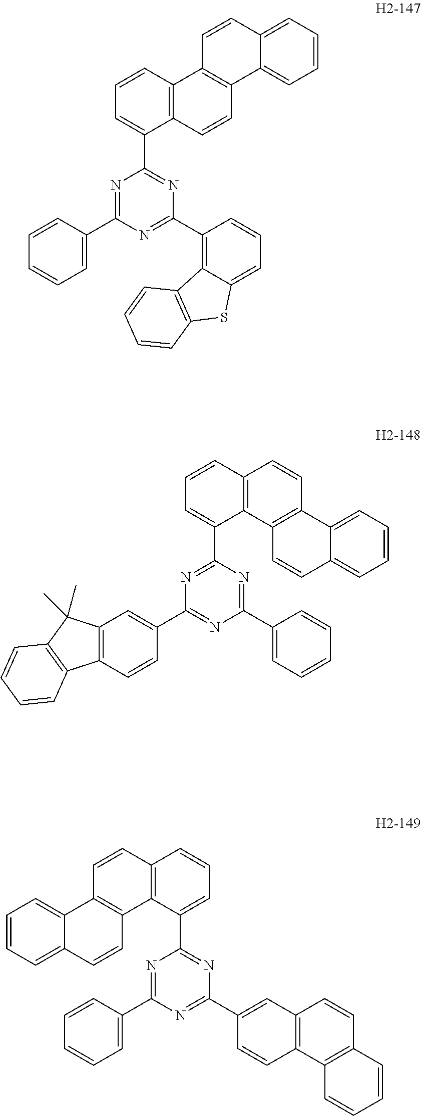 [SMILES: c1ccc(-c2nc(-c3cccc4c3ccc3c5ccccc5ccc43)nc(-c3cccc4sc5ccccc5c34)n2)cc1 ; CC1(C)c2ccccc2-c2ccc(-c3nc(-c4ccccc4)nc(-c4cccc5ccc6c7ccccc7ccc6c45)n3)cc21 ; c1ccc(-c2nc(-c3ccc4c(ccc5ccccc54)c3)nc(-c3cccc4ccc5c6ccccc6ccc5c34)n2)cc1]

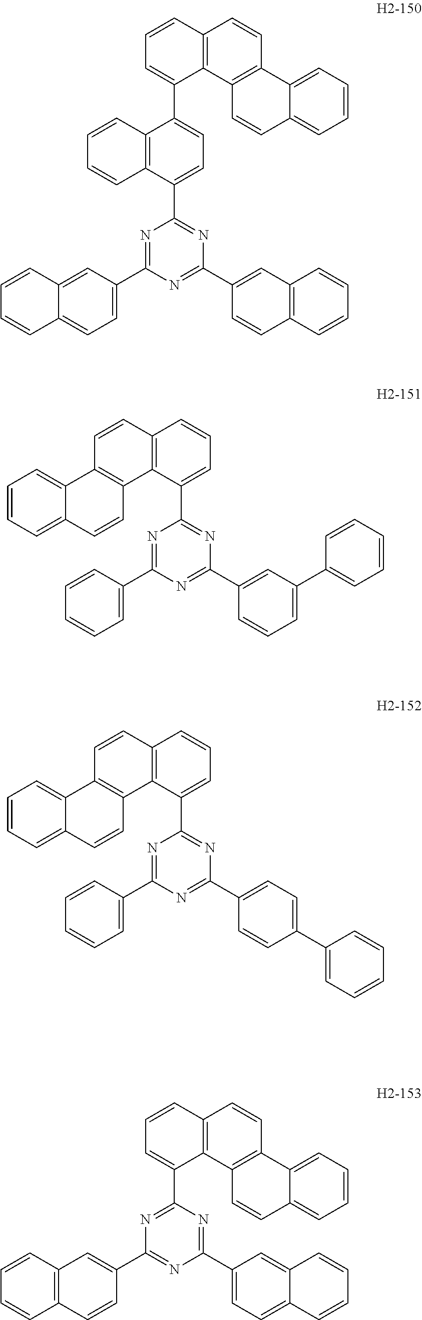 [SMILES: c1ccc2cc(-c3nc(-c4ccc5ccccc5c4)nc(-c4ccc(-c5cccc6ccc7c8ccccc8ccc7c56)c5ccccc45)n3)ccc2c1 ; c1ccc(-c2cccc(-c3nc(-c4ccccc4)nc(-c4cccc5ccc6c7ccccc7ccc6c45)n3)c2)cc1 ; c1ccc(-c2ccc(-c3nc(-c4ccccc4)nc(-c4cccc5ccc6c7ccccc7ccc6c45)n3)cc2)cc1 ; c1ccc2cc(-c3nc(-c4ccc5ccccc5c4)nc(-c4cccc5ccc6c7ccccc7ccc6c45)n3)ccc2c1]

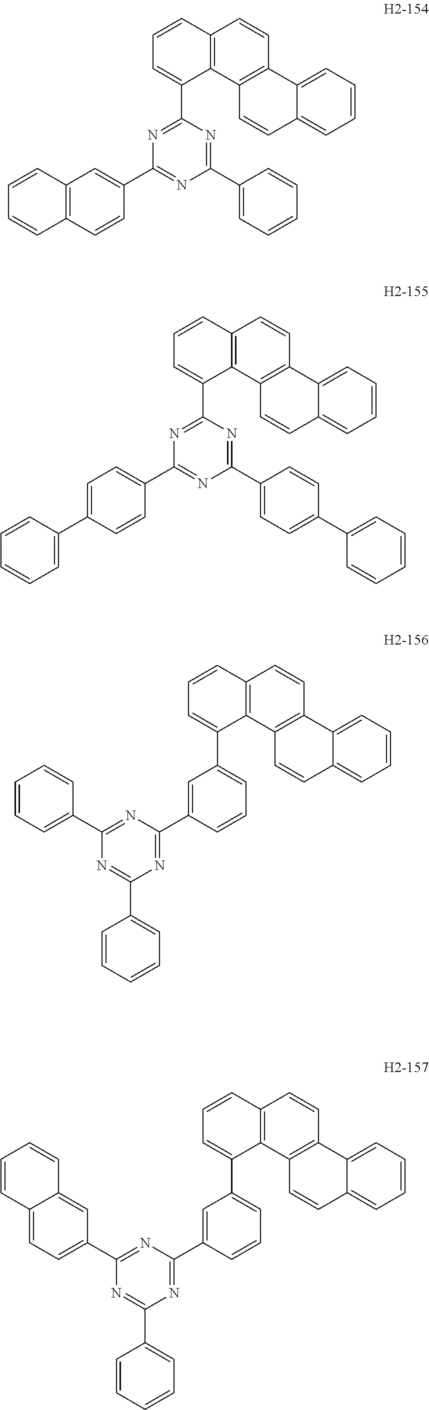 [SMILES: c1ccc(-c2nc(-c3ccc4ccccc4c3)nc(-c3cccc4ccc5c6ccccc6ccc5c34)n2)cc1 ; c1ccc(-c2ccc(-c3nc(-c4ccc(-c5ccccc5)cc4)nc(-c4cccc5ccc6c7ccccc7ccc6c45)n3)cc2)cc1 ; c1ccc(-c2nc(-c3ccccc3)nc(-c3cccc(-c4cccc5ccc6c7ccccc7ccc6c45)c3)n2)cc1 ; c1ccc(-c2nc(-c3cccc(-c4cccc5ccc6c7ccccc7ccc6c45)c3)nc(-c3ccc4ccccc4c3)n2)cc1]

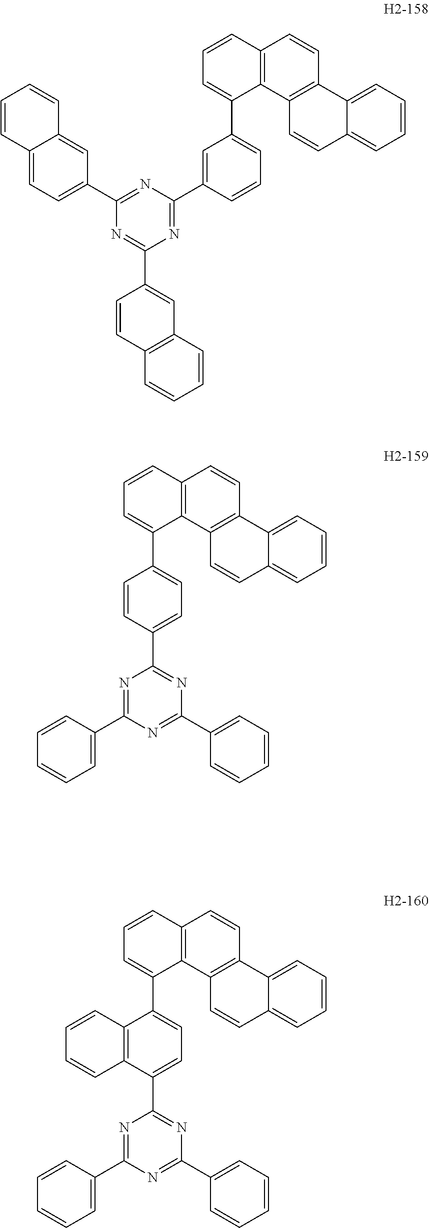 [SMILES: c1cc(-c2nc(-c3ccc4ccccc4c3)nc(-c3ccc4ccccc4c3)n2)cc(-c2cccc3ccc4c5ccccc5ccc4c23)c1 ; c1ccc(-c2nc(-c3ccccc3)nc(-c3ccc(-c4cccc5ccc6c7ccccc7ccc6c45)cc3)n2)cc1 ; c1ccc(-c2nc(-c3ccccc3)nc(-c3ccc(-c4cccc5ccc6c7ccccc7ccc6c45)c4ccccc34)n2)cc1]

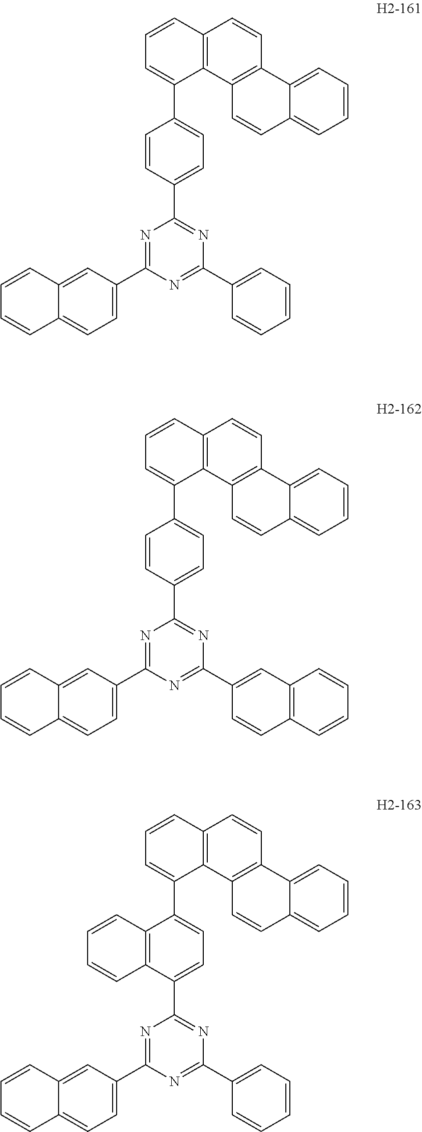 [SMILES: c1ccc(-c2nc(-c3ccc(-c4cccc5ccc6c7ccccc7ccc6c45)cc3)nc(-c3ccc4ccccc4c3)n2)cc1 ; c1ccc2cc(-c3nc(-c4ccc(-c5cccc6ccc7c8ccccc8ccc7c56)cc4)nc(-c4ccc5ccccc5c4)n3)ccc2c1 ; c1ccc(-c2nc(-c3ccc4ccccc4c3)nc(-c3ccc(-c4cccc5ccc6c7ccccc7ccc6c45)c4ccccc34)n2)cc1]

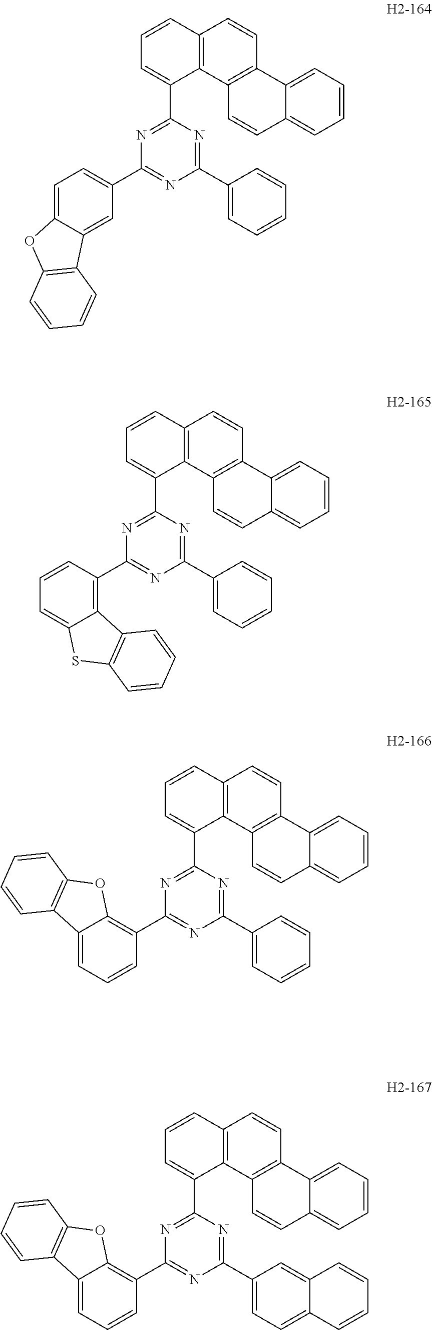 [SMILES: c1ccc(-c2nc(-c3ccc4oc5ccccc5c4c3)nc(-c3cccc4ccc5c6ccccc6ccc5c34)n2)cc1 ; c1ccc(-c2nc(-c3cccc4ccc5c6ccccc6ccc5c34)nc(-c3cccc4sc5ccccc5c34)n2)cc1 ; c1ccc(-c2nc(-c3cccc4c3oc3ccccc34)nc(-c3cccc4ccc5c6ccccc6ccc5c34)n2)cc1 ; c1ccc2cc(-c3nc(-c4cccc5c4oc4ccccc45)nc(-c4cccc5ccc6c7ccccc7ccc6c45)n3)ccc2c1]

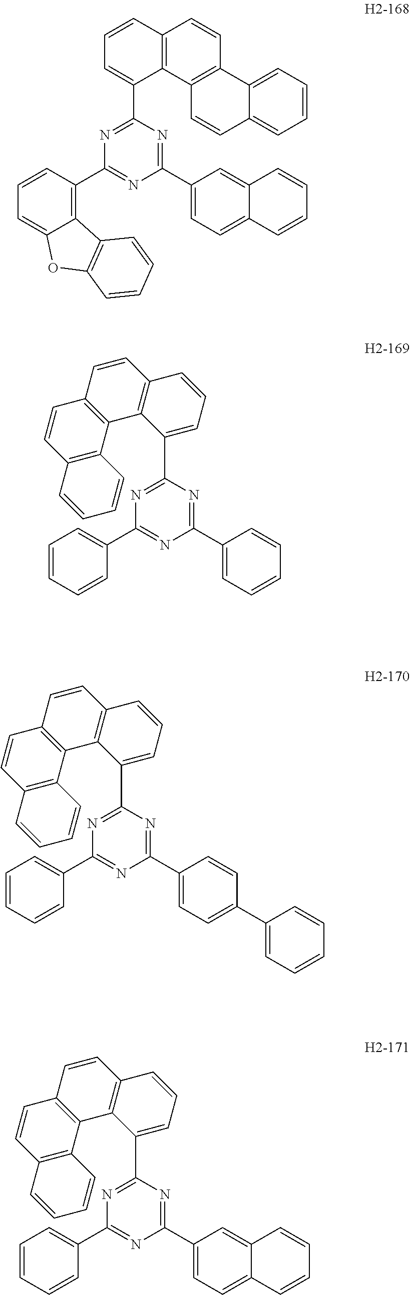 [SMILES: c1ccc2cc(-c3nc(-c4cccc5ccc6c7ccccc7ccc6c45)nc(-c4cccc5oc6ccccc6c45)n3)ccc2c1 ; c1ccc(-c2nc(-c3ccccc3)nc(-c3cccc4ccc5ccc6ccccc6c5c34)n2)cc1 ; c1ccc(-c2ccc(-c3nc(-c4ccccc4)nc(-c4cccc5ccc6ccc7ccccc7c6c45)n3)cc2)cc1 ; c1ccc(-c2nc(-c3ccc4ccccc4c3)nc(-c3cccc4ccc5ccc6ccccc6c5c34)n2)cc1]

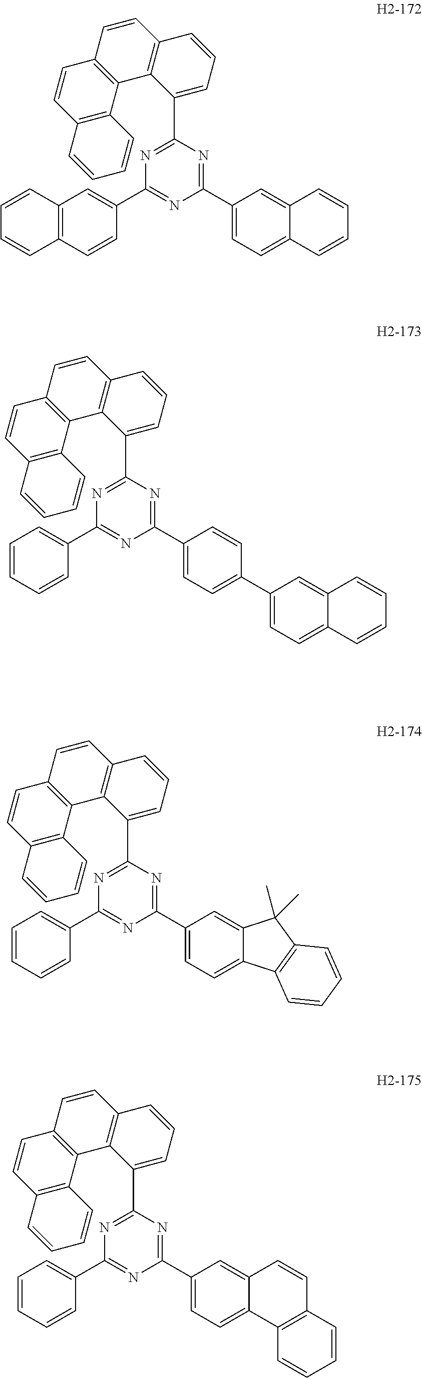 [SMILES: c1ccc2cc(-c3nc(-c4ccc5ccccc5c4)nc(-c4cccc5ccc6ccc7ccccc7c6c45)n3)ccc2c1 ; c1ccc(-c2nc(-c3ccc(-c4ccc5ccccc5c4)cc3)nc(-c3cccc4ccc5ccc6ccccc6c5c34)n2)cc1 ; CC1(C)c2ccccc2-c2ccc(-c3nc(-c4ccccc4)nc(-c4cccc5ccc6ccc7ccccc7c6c45)n3)cc21 ; c1ccc(-c2nc(-c3ccc4c(ccc5ccccc54)c3)nc(-c3cccc4ccc5ccc6ccccc6c5c34)n2)cc1]

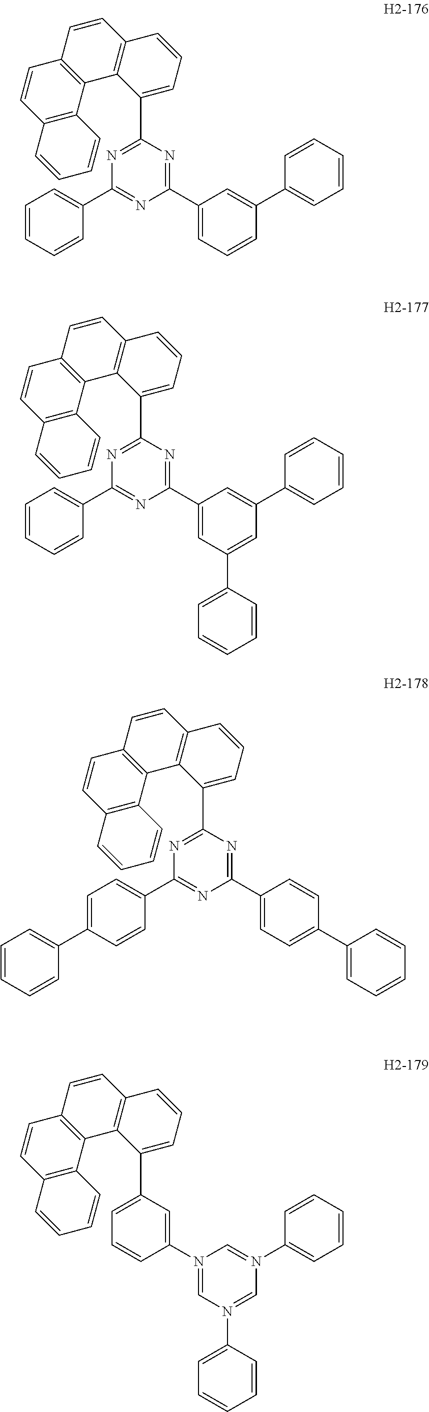

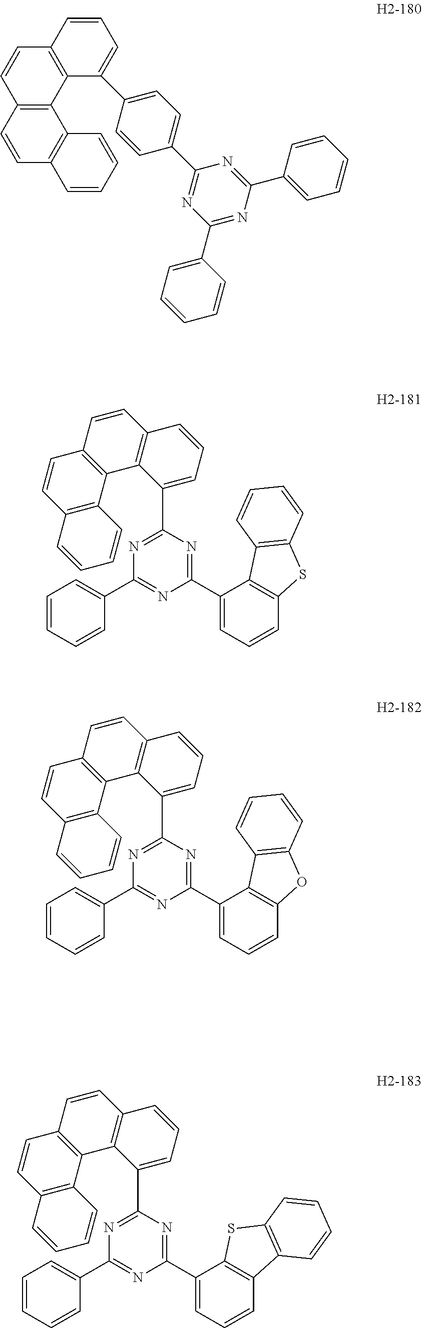 [SMILES: c1ccc(-c2nc(-c3ccccc3)nc(-c3ccc(-c4cccc5ccc6ccc7ccccc7c6c45)cc3)n2)cc1 ; c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1]

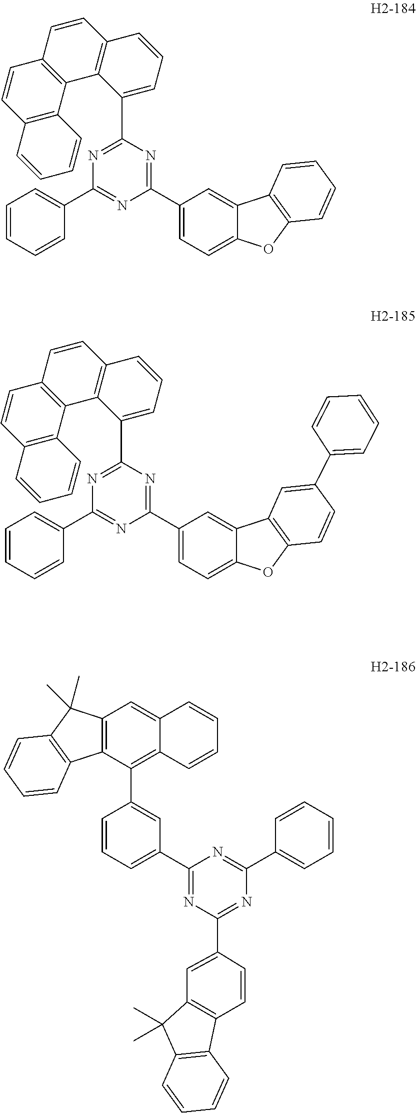 [SMILES: c1ccc(-c2nc(-c3ccc4oc5ccccc5c4c3)nc(-c3cccc4ccc5ccc6ccccc6c5c34)n2)cc1 ; c1ccc(-c2ccc3oc4ccc(-c5nc(-c6ccccc6)nc(-c6cccc7ccc8ccc9ccccc9c8c67)n5)cc4c3c2)cc1 ; CC1(C)c2ccccc2-c2ccc(-c3nc(-c4ccccc4)nc(-c4cccc(-c5c6c(cc7ccccc57)C(C)(C)c5ccccc5-6)c4)n3)cc21]

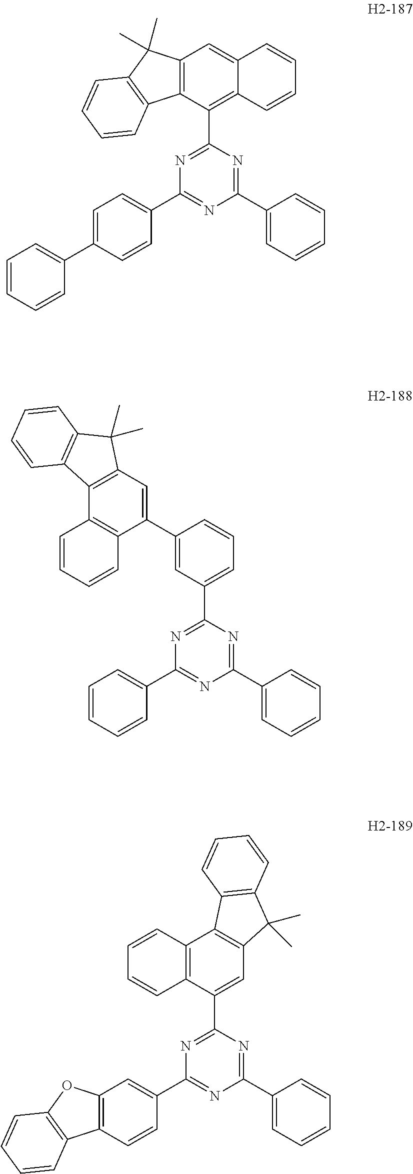 [SMILES: CC1(C)c2ccccc2-c2c1cc1ccccc1c2-c1nc(-c2ccccc2)nc(-c2ccc(-c3ccccc3)cc2)n1 ; CC1(C)c2ccccc2-c2c1cc(-c1cccc(-c3nc(-c4ccccc4)nc(-c4ccccc4)n3)c1)c1ccccc21 ; CC1(C)c2ccccc2-c2c1cc(-c1nc(-c3ccccc3)nc(-c3ccc4c(c3)oc3ccccc34)n1)c1ccccc21]

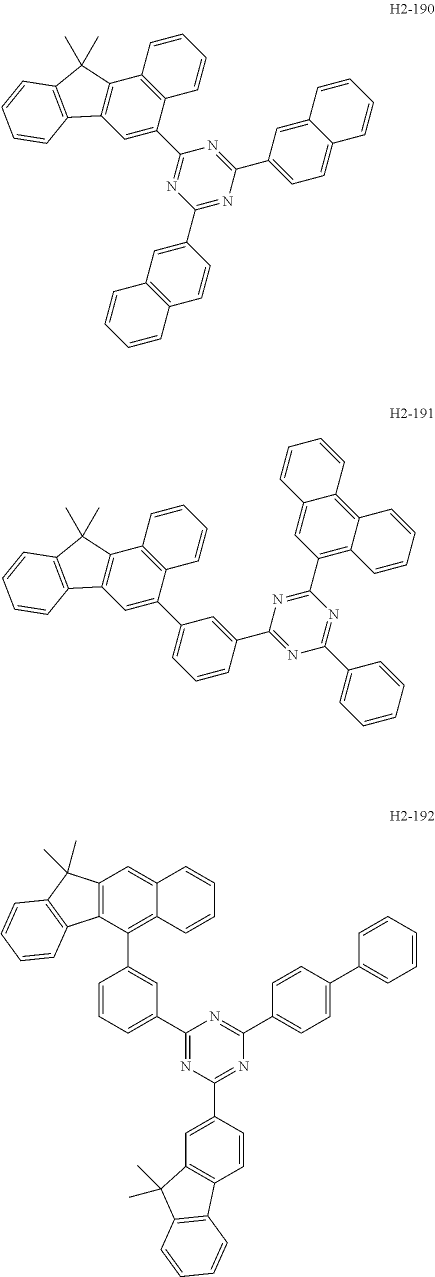 [SMILES: CC1(C)c2ccccc2-c2cc(-c3nc(-c4ccc5ccccc5c4)nc(-c4ccc5ccccc5c4)n3)c3ccccc3c21 ; CC1(C)c2ccccc2-c2cc(-c3cccc(-c4nc(-c5ccccc5)nc(-c5cc6ccccc6c6ccccc56)n4)c3)c3ccccc3c21 ; CC1(C)c2ccccc2-c2ccc(-c3nc(-c4ccc(-c5ccccc5)cc4)nc(-c4cccc(-c5c6c(cc7ccccc57)C(C)(C)c5ccccc5-6)c4)n3)cc21]

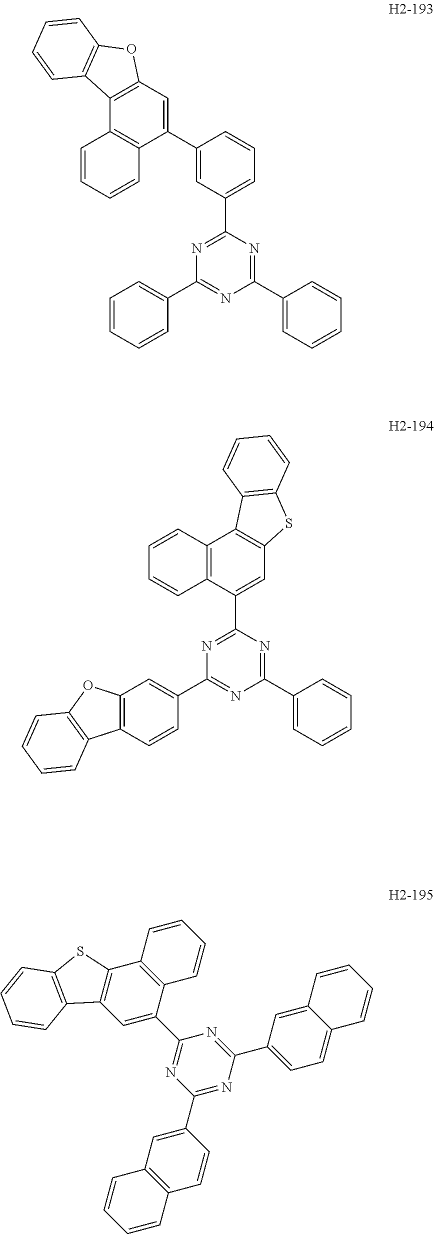 [SMILES: c1ccc(-c2nc(-c3ccccc3)nc(-c3cccc(-c4cc5oc6ccccc6c5c5ccccc45)c3)n2)cc1 ; c1ccc(-c2nc(-c3ccc4c(c3)oc3ccccc34)nc(-c3cc4sc5ccccc5c4c4ccccc34)n2)cc1 ; c1ccc2cc(-c3nc(-c4ccc5ccccc5c4)nc(-c4cc5c6ccccc6sc5c5ccccc45)n3)ccc2c1]

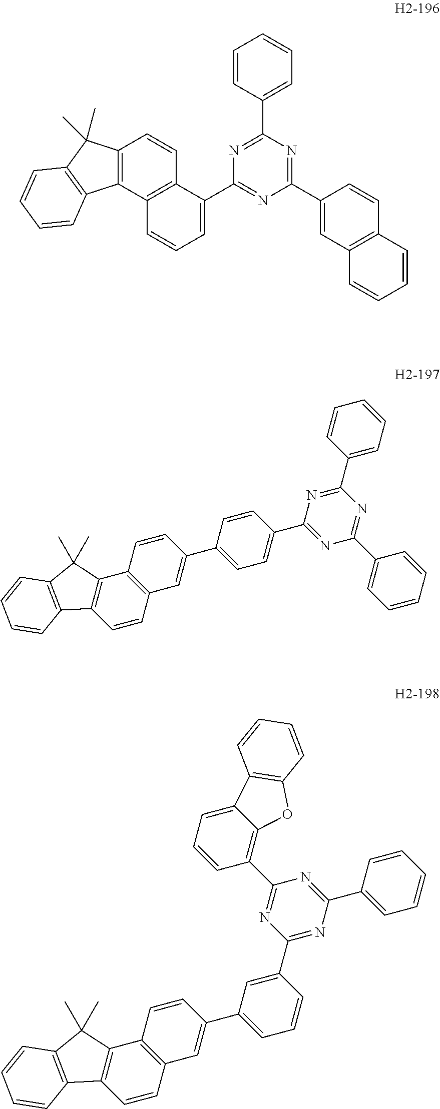 [SMILES: CC1(C)c2ccccc2-c2c1ccc1c(-c3nc(-c4ccccc4)nc(-c4ccc5ccccc5c4)n3)cccc21 ; CC1(C)c2ccccc2-c2ccc3cc(-c4ccc(-c5nc(-c6ccccc6)nc(-c6ccccc6)n5)cc4)ccc3c21 ; CC1(C)c2ccccc2-c2ccc3cc(-c4cccc(-c5nc(-c6ccccc6)nc(-c6cccc7c6oc6ccccc67)n5)c4)ccc3c21]

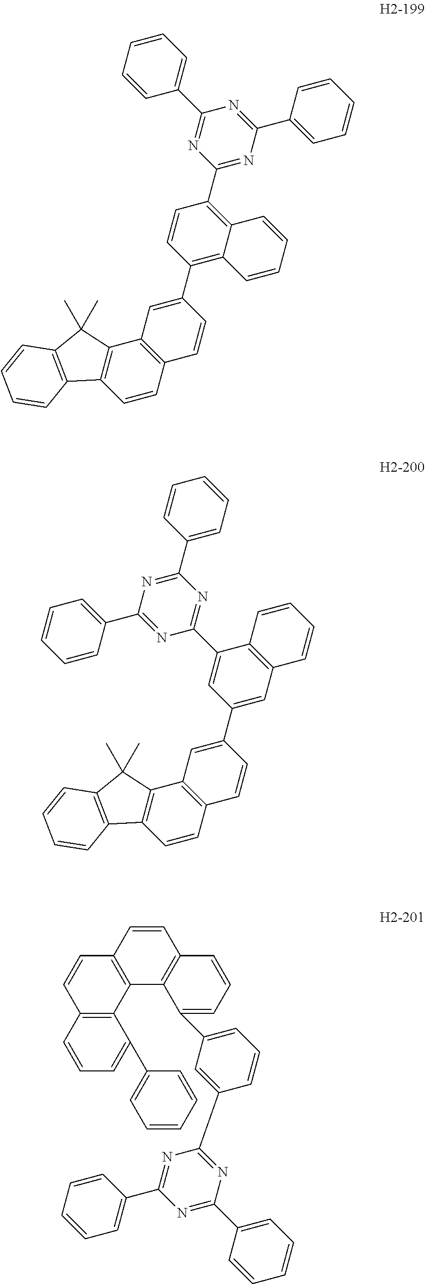 [SMILES: CC1(C)c2ccccc2-c2ccc3ccc(-c4ccc(-c5nc(-c6ccccc6)nc(-c6ccccc6)n5)c5ccccc45)cc3c21 ; CC1(C)c2ccccc2-c2ccc3ccc(-c4cc(-c5nc(-c6ccccc6)nc(-c6ccccc6)n5)c5ccccc5c4)cc3c21 ; c1ccc(-c2nc(-c3ccccc3)nc(-c3cccc(-c4cccc5ccc6ccc7cccc(-c8ccccc8)c7c6c45)c3)n2)cc1]

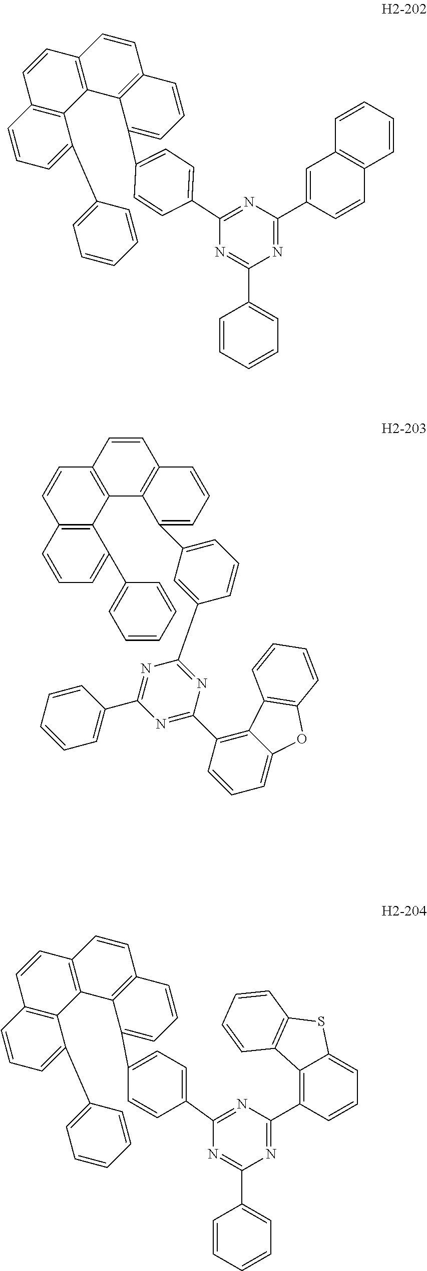 [SMILES: c1ccc(-c2nc(-c3ccc(-c4cccc5ccc6ccc7cccc(-c8ccccc8)c7c6c45)cc3)nc(-c3ccc4ccccc4c3)n2)cc1 ; c1ccc(-c2nc(-c3cccc(-c4cccc5ccc6ccc7cccc(-c8ccccc8)c7c6c45)c3)nc(-c3cccc4oc5ccccc5c34)n2)cc1 ; c1ccc(-c2nc(-c3ccc(-c4cccc5ccc6ccc7cccc(-c8ccccc8)c7c6c45)cc3)nc(-c3cccc4sc5ccccc5c34)n2)cc1]

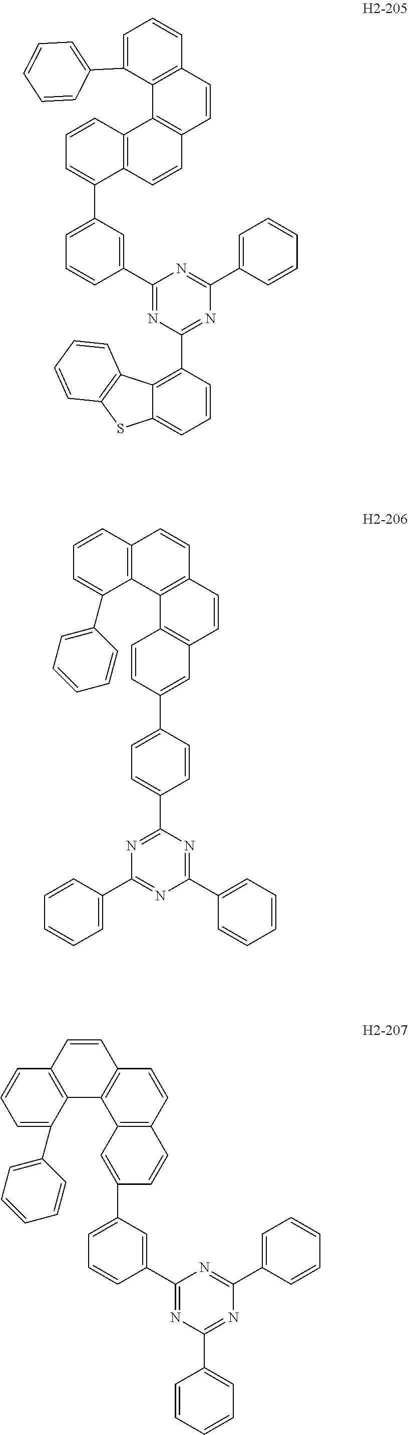 [SMILES: c1ccc(-c2nc(-c3cccc(-c4cccc5c4ccc4ccc6cccc(-c7ccccc7)c6c45)c3)nc(-c3cccc4sc5ccccc5c34)n2)cc1 ; c1ccc(-c2nc(-c3ccccc3)nc(-c3ccc(-c4ccc5c(ccc6ccc7cccc(-c8ccccc8)c7c65)c4)cc3)n2)cc1 ; c1ccc(-c2nc(-c3ccccc3)nc(-c3cccc(-c4ccc5ccc6ccc7cccc(-c8ccccc8)c7c6c5c4)c3)n2)cc1]

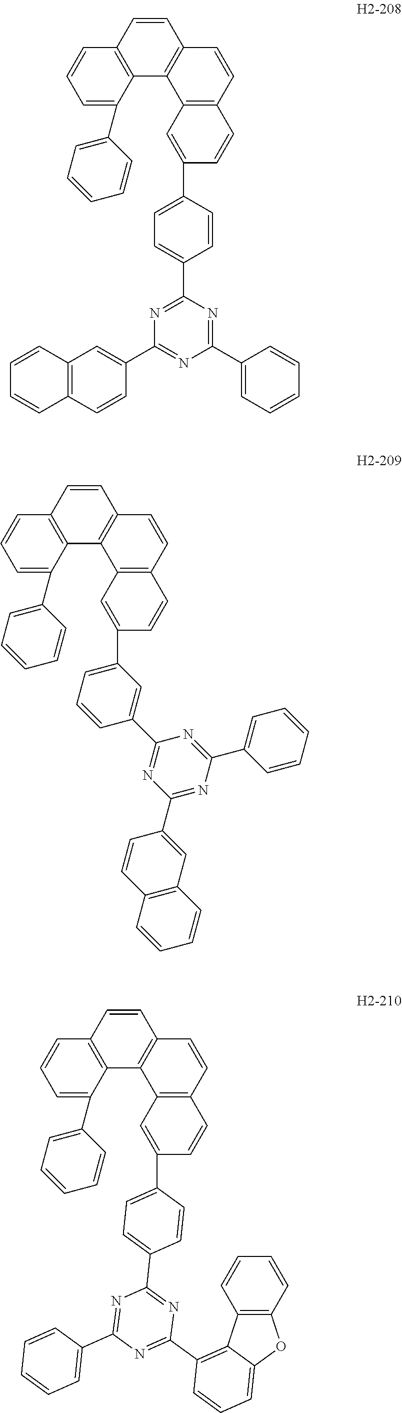 [SMILES: c1ccc(-c2nc(-c3ccc(-c4ccc5ccc6ccc7cccc(-c8ccccc8)c7c6c5c4)cc3)nc(-c3ccc4ccccc4c3)n2)cc1 ; c1ccc(-c2nc(-c3cccc(-c4ccc5ccc6ccc7cccc(-c8ccccc8)c7c6c5c4)c3)nc(-c3ccc4ccccc4c3)n2)cc1 ; c1ccc(-c2nc(-c3ccc(-c4ccc5ccc6ccc7cccc(-c8ccccc8)c7c6c5c4)cc3)nc(-c3cccc4oc5ccccc5c34)n2)cc1]

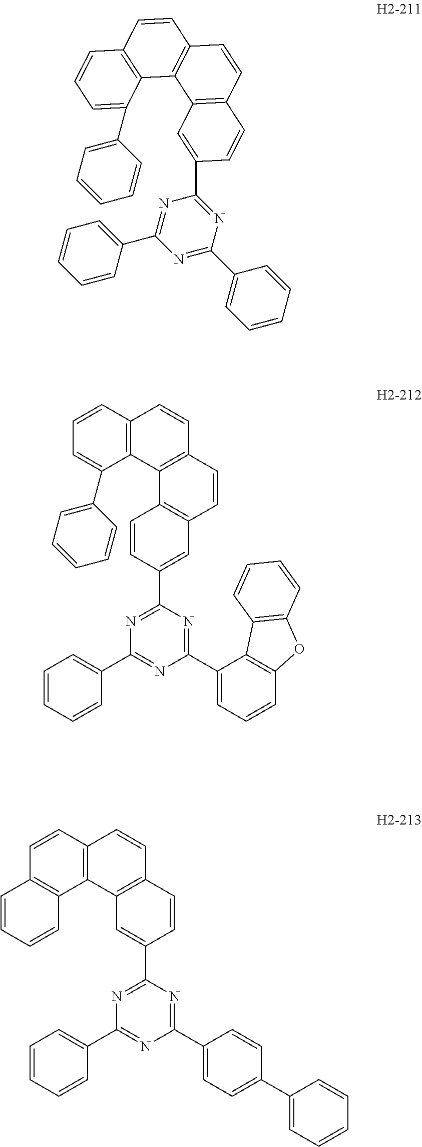 [SMILES: c1ccc(-c2nc(-c3ccccc3)nc(-c3ccc4ccc5ccc6cccc(-c7ccccc7)c6c5c4c3)n2)cc1 ; c1ccc(-c2nc(-c3ccc4c(ccc5ccc6cccc(-c7ccccc7)c6c54)c3)nc(-c3cccc4oc5ccccc5c34)n2)cc1 ; c1ccc(-c2ccc(-c3nc(-c4ccccc4)nc(-c4ccc5ccc6ccc7ccccc7c6c5c4)n3)cc2)cc1]

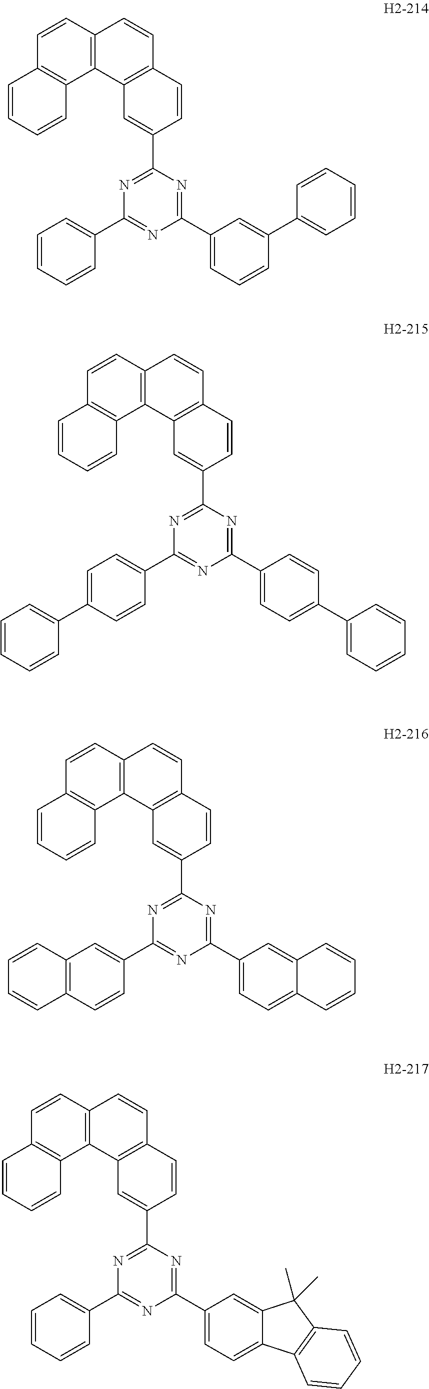 [SMILES: c1ccc(-c2cccc(-c3nc(-c4ccccc4)nc(-c4ccc5ccc6ccc7ccccc7c6c5c4)n3)c2)cc1 ; c1ccc(-c2ccc(-c3nc(-c4ccc(-c5ccccc5)cc4)nc(-c4ccc5ccc6ccc7ccccc7c6c5c4)n3)cc2)cc1 ; c1ccc2cc(-c3nc(-c4ccc5ccccc5c4)nc(-c4ccc5ccc6ccc7ccccc7c6c5c4)n3)ccc2c1 ; CC1(C)c2ccccc2-c2ccc(-c3nc(-c4ccccc4)nc(-c4ccc5ccc6ccc7ccccc7c6c5c4)n3)cc21]

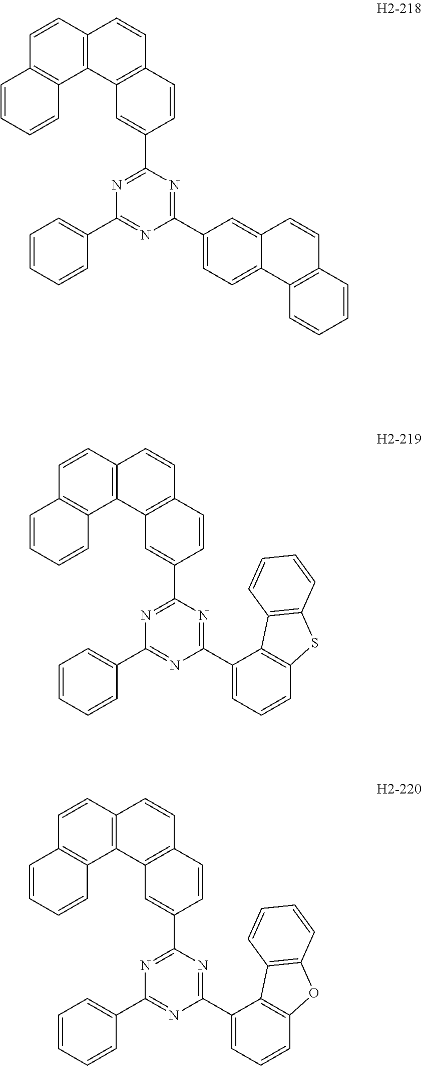 [SMILES: c1ccc(-c2nc(-c3ccc4c(ccc5ccccc54)c3)nc(-c3ccc4ccc5ccc6ccccc6c5c4c3)n2)cc1 ; c1ccc(-c2nc(-c3ccc4ccc5ccc6ccccc6c5c4c3)nc(-c3cccc4sc5ccccc5c34)n2)cc1 ; c1ccc(-c2nc(-c3ccc4ccc5ccc6ccccc6c5c4c3)nc(-c3cccc4oc5ccccc5c34)n2)cc1]

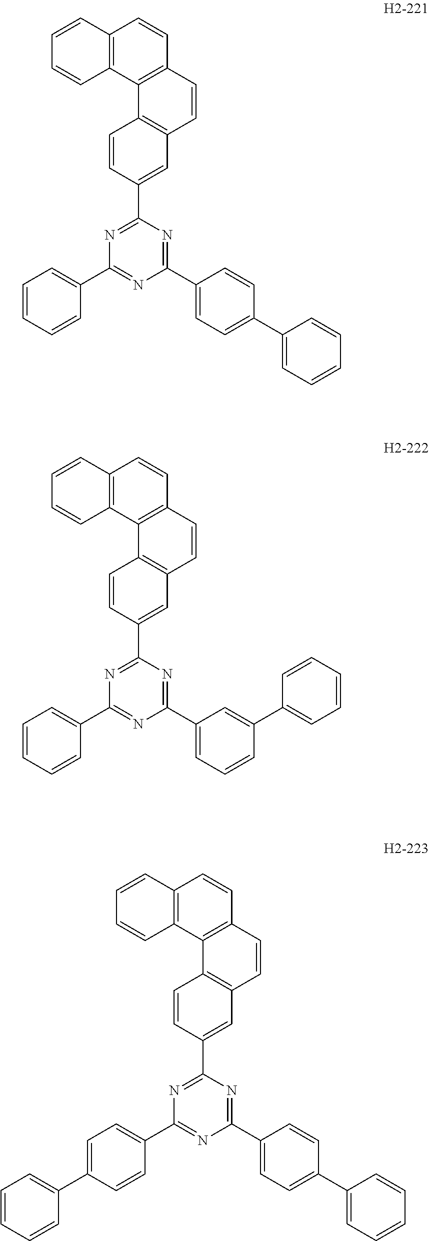 [SMILES: c1ccc(-c2ccc(-c3nc(-c4ccccc4)nc(-c4ccc5c(ccc6ccc7ccccc7c65)c4)n3)cc2)cc1 ; c1ccc(-c2cccc(-c3nc(-c4ccccc4)nc(-c4ccc5c(ccc6ccc7ccccc7c65)c4)n3)c2)cc1 ; c1ccc(-c2ccc(-c3nc(-c4ccc(-c5ccccc5)cc4)nc(-c4ccc5c(ccc6ccc7ccccc7c65)c4)n3)cc2)cc1]

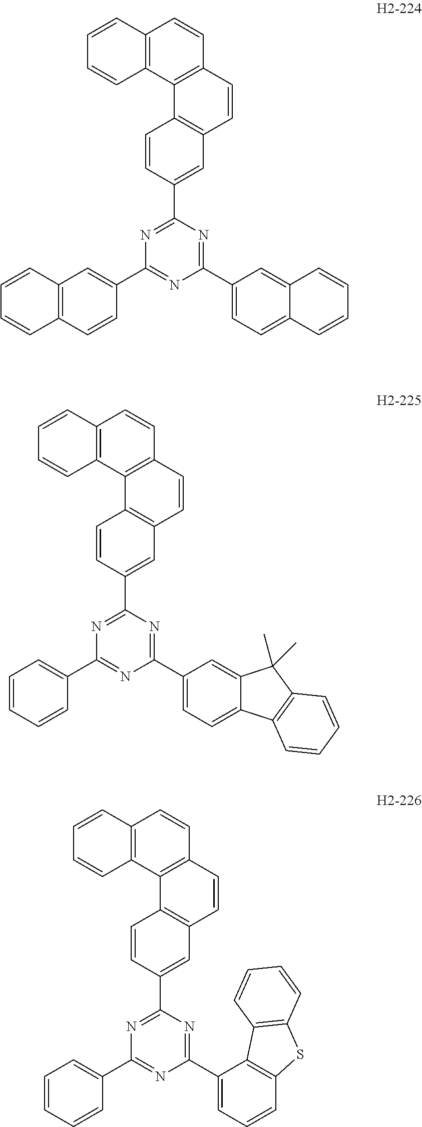 [SMILES: c1ccc2cc(-c3nc(-c4ccc5ccccc5c4)nc(-c4ccc5c(ccc6ccc7ccccc7c65)c4)n3)ccc2c1 ; CC1(C)c2ccccc2-c2ccc(-c3nc(-c4ccccc4)nc(-c4ccc5c(ccc6ccc7ccccc7c65)c4)n3)cc21 ; c1ccc(-c2nc(-c3ccc4c(ccc5ccc6ccccc6c54)c3)nc(-c3cccc4sc5ccccc5c34)n2)cc1]

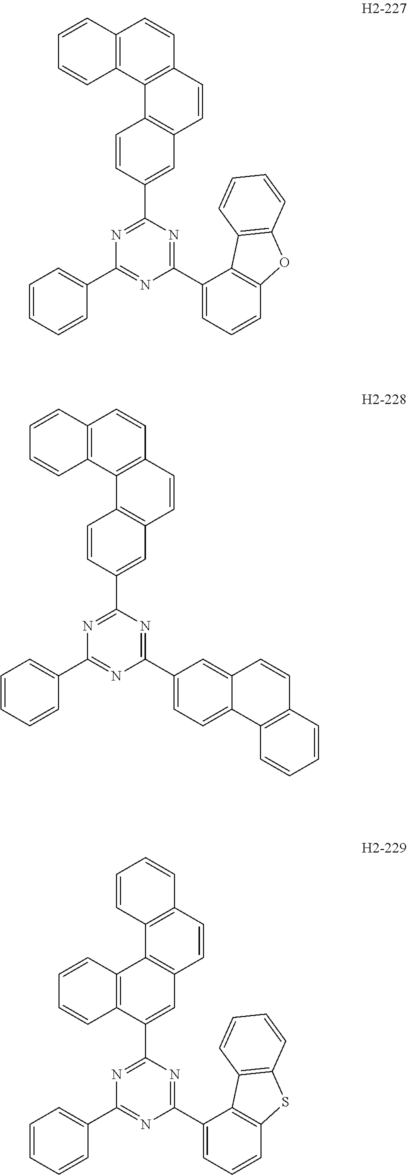 [SMILES: c1ccc(-c2nc(-c3ccc4c(ccc5ccc6ccccc6c54)c3)nc(-c3cccc4oc5ccccc5c34)n2)cc1 ; c1ccc(-c2nc(-c3ccc4c(ccc5ccccc54)c3)nc(-c3ccc4c(ccc5ccc6ccccc6c54)c3)n2)cc1 ; c1ccc(-c2nc(-c3cc4ccc5ccccc5c4c4ccccc34)nc(-c3cccc4sc5ccccc5c34)n2)cc1]

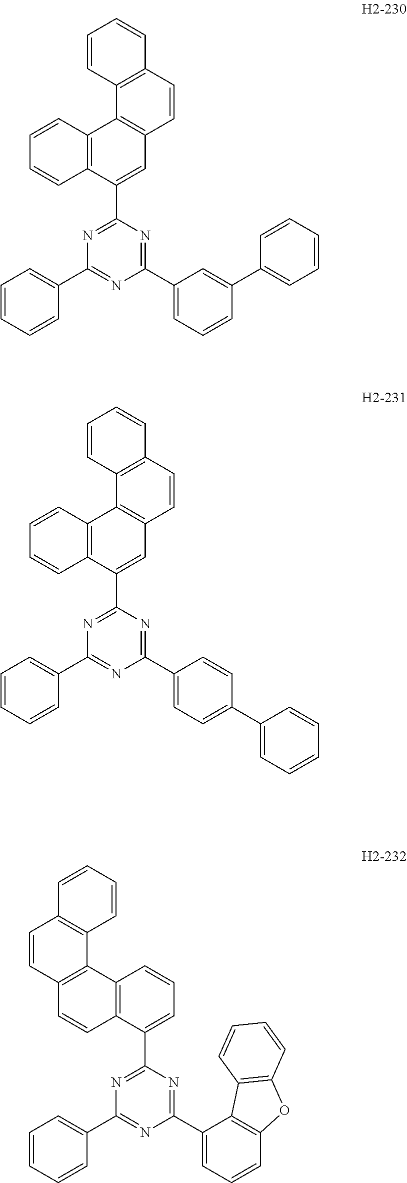 [SMILES: c1ccc(-c2cccc(-c3nc(-c4ccccc4)nc(-c4cc5ccc6ccccc6c5c5ccccc45)n3)c2)cc1 ; c1ccc(-c2ccc(-c3nc(-c4ccccc4)nc(-c4cc5ccc6ccccc6c5c5ccccc45)n3)cc2)cc1 ; c1ccc(-c2nc(-c3cccc4c3ccc3ccc5ccccc5c34)nc(-c3cccc4oc5ccccc5c34)n2)cc1]

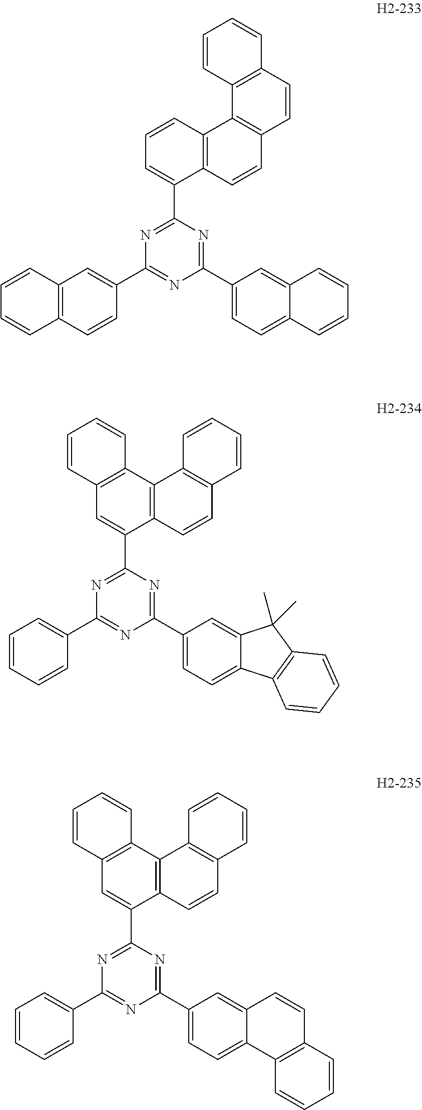 [SMILES: c1ccc2cc(-c3nc(-c4ccc5ccccc5c4)nc(-c4cccc5c4ccc4ccc6ccccc6c45)n3)ccc2c1 ; CC1(C)c2ccccc2-c2ccc(-c3nc(-c4ccccc4)nc(-c4cc5ccccc5c5c4ccc4ccccc45)n3)cc21 ; c1ccc(-c2nc(-c3ccc4c(ccc5ccccc54)c3)nc(-c3cc4ccccc4c4c3ccc3ccccc34)n2)cc1]

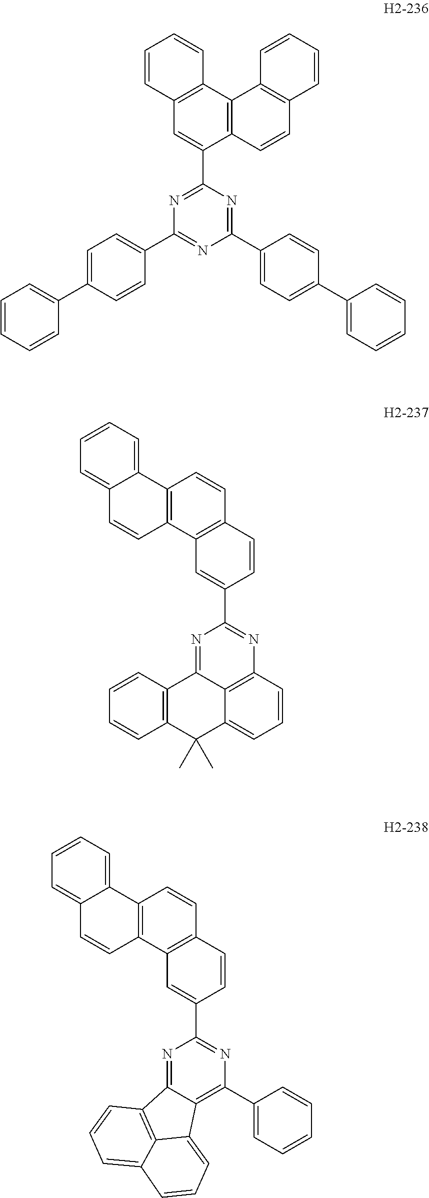 [SMILES: c1ccc(-c2ccc(-c3nc(-c4ccc(-c5ccccc5)cc4)nc(-c4cc5ccccc5c5c4ccc4ccccc45)n3)cc2)cc1 ; CC1(C)c2ccccc2-c2nc(-c3ccc4ccc5c6ccccc6ccc5c4c3)nc3cccc1c23 ; c1ccc(-c2nc(-c3ccc4ccc5c6ccccc6ccc5c4c3)nc3c2-c2cccc4cccc-3c24)cc1]

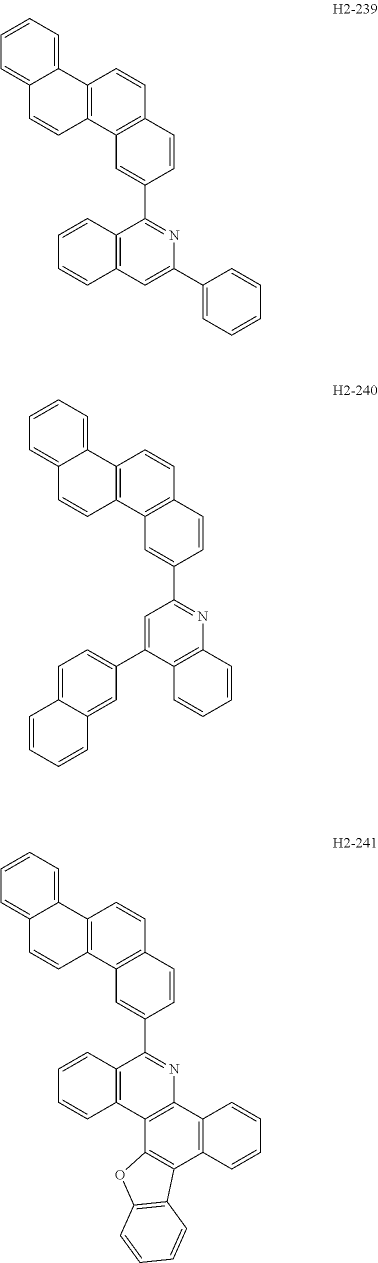 [SMILES: c1ccc(-c2cc3ccccc3c(-c3ccc4ccc5c6ccccc6ccc5c4c3)n2)cc1 ; c1ccc2cc(-c3cc(-c4ccc5ccc6c7ccccc7ccc6c5c4)nc4ccccc34)ccc2c1 ; c1ccc2c(c1)ccc1c3cc(-c4nc5c6ccccc6c6c7ccccc7oc6c5c5ccccc45)ccc3ccc21]

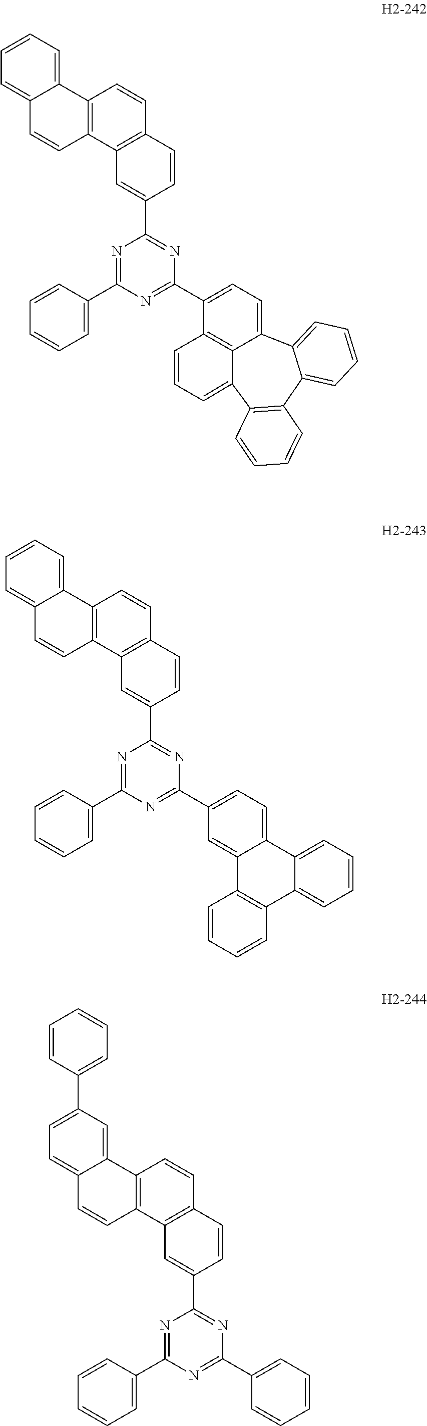 [SMILES: c1ccc(-c2nc(-c3ccc4ccc5c6ccccc6ccc5c4c3)nc(-c3ccc4c5c(cccc35)-c3ccccc3-c3ccccc3-4)n2)cc1 ; c1ccc(-c2nc(-c3ccc4ccc5c6ccccc6ccc5c4c3)nc(-c3ccc4c5ccccc5c5ccccc5c4c3)n2)cc1 ; c1ccc(-c2ccc3ccc4c5cc(-c6nc(-c7ccccc7)nc(-c7ccccc7)n6)ccc5ccc4c3c2)cc1]

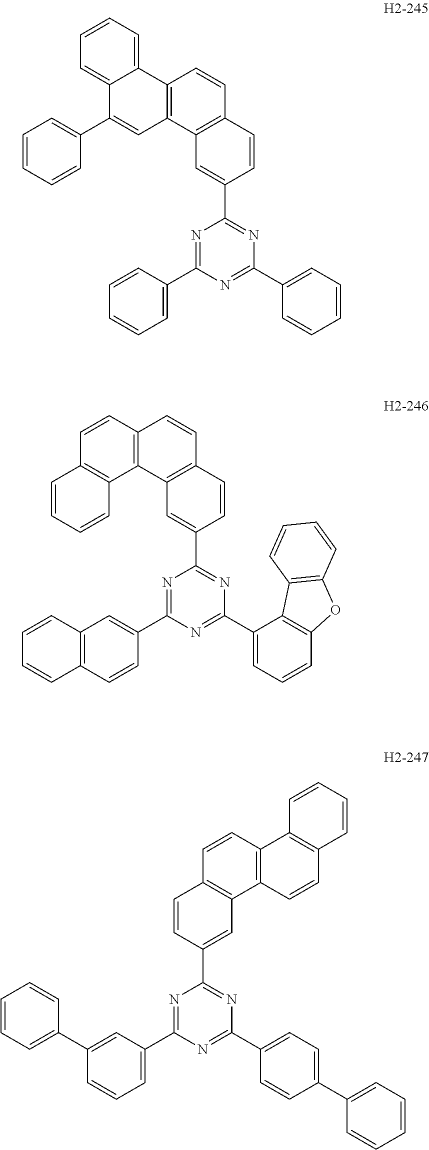 [SMILES: c1ccc(-c2nc(-c3ccccc3)nc(-c3ccc4ccc5c6ccccc6c(-c6ccccc6)cc5c4c3)n2)cc1 ; c1ccc2cc(-c3nc(-c4ccc5ccc6ccc7ccccc7c6c5c4)nc(-c4cccc5oc6ccccc6c45)n3)ccc2c1 ; c1ccc(-c2ccc(-c3nc(-c4cccc(-c5ccccc5)c4)nc(-c4ccc5ccc6c7ccccc7ccc6c5c4)n3)cc2)cc1]

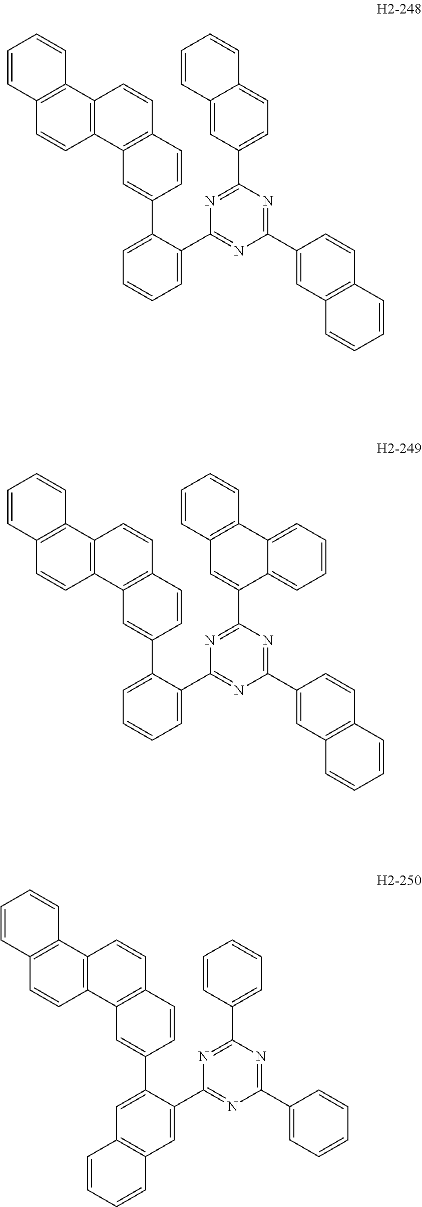 [SMILES: c1ccc(-c2nc(-c3ccc4ccccc4c3)nc(-c3ccc4ccccc4c3)n2)c(-c2ccc3ccc4c5ccccc5ccc4c3c2)c1 ; c1ccc(-c2nc(-c3ccc4ccccc4c3)nc(-c3cc4ccccc4c4ccccc34)n2)c(-c2ccc3ccc4c5ccccc5ccc4c3c2)c1 ; c1ccc(-c2nc(-c3ccccc3)nc(-c3cc4ccccc4cc3-c3ccc4ccc5c6ccccc6ccc5c4c3)n2)cc1]

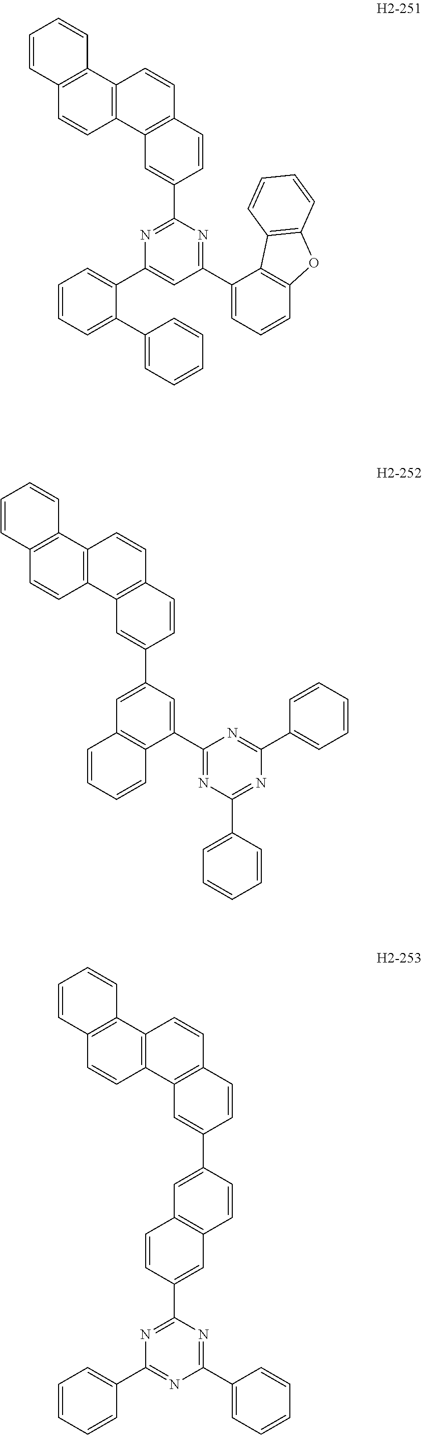 [SMILES: c1ccc(-c2ccccc2-c2cc(-c3cccc4oc5ccccc5c34)nc(-c3ccc4ccc5c6ccccc6ccc5c4c3)n2)cc1 ; c1ccc(-c2nc(-c3ccccc3)nc(-c3cc(-c4ccc5ccc6c7ccccc7ccc6c5c4)cc4ccccc34)n2)cc1 ; c1ccc(-c2nc(-c3ccccc3)nc(-c3ccc4cc(-c5ccc6ccc7c8ccccc8ccc7c6c5)ccc4c3)n2)cc1]

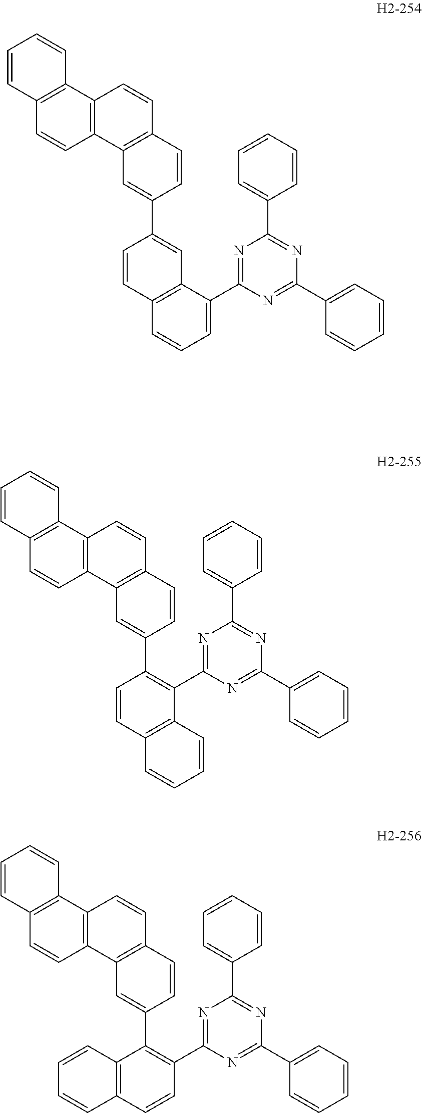 [SMILES: c1ccc(-c2nc(-c3ccccc3)nc(-c3cccc4ccc(-c5ccc6ccc7c8ccccc8ccc7c6c5)cc34)n2)cc1 ; c1ccc(-c2nc(-c3ccccc3)nc(-c3c(-c4ccc5ccc6c7ccccc7ccc6c5c4)ccc4ccccc34)n2)cc1 ; c1ccc(-c2nc(-c3ccccc3)nc(-c3ccc4ccccc4c3-c3ccc4ccc5c6ccccc6ccc5c4c3)n2)cc1]

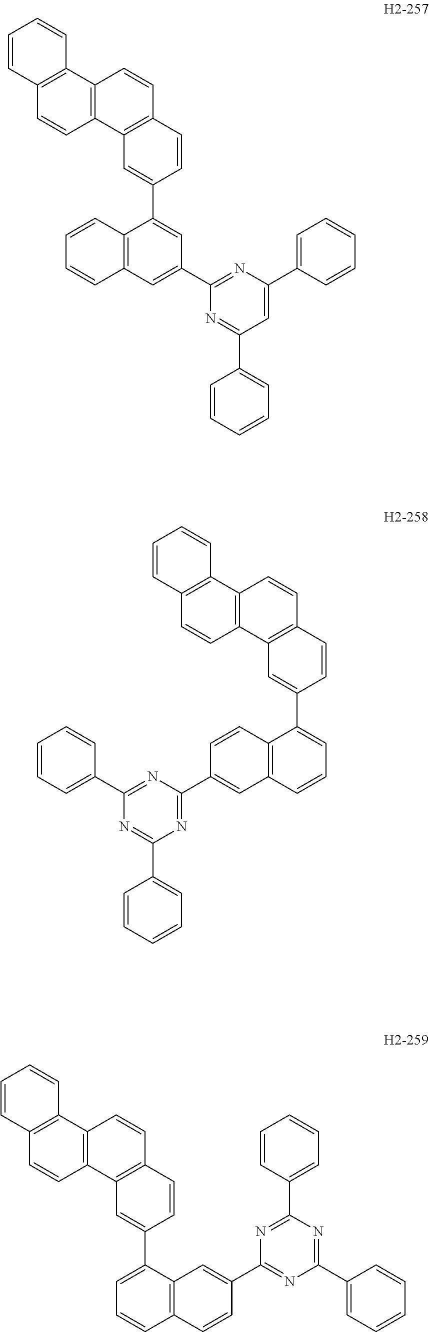 [SMILES: c1ccc(-c2cc(-c3ccccc3)nc(-c3cc(-c4ccc5ccc6c7ccccc7ccc6c5c4)c4ccccc4c3)n2)cc1 ; c1ccc(-c2nc(-c3ccccc3)nc(-c3ccc4c(-c5ccc6ccc7c8ccccc8ccc7c6c5)cccc4c3)n2)cc1 ; c1ccc(-c2nc(-c3ccccc3)nc(-c3ccc4cccc(-c5ccc6ccc7c8ccccc8ccc7c6c5)c4c3)n2)cc1]

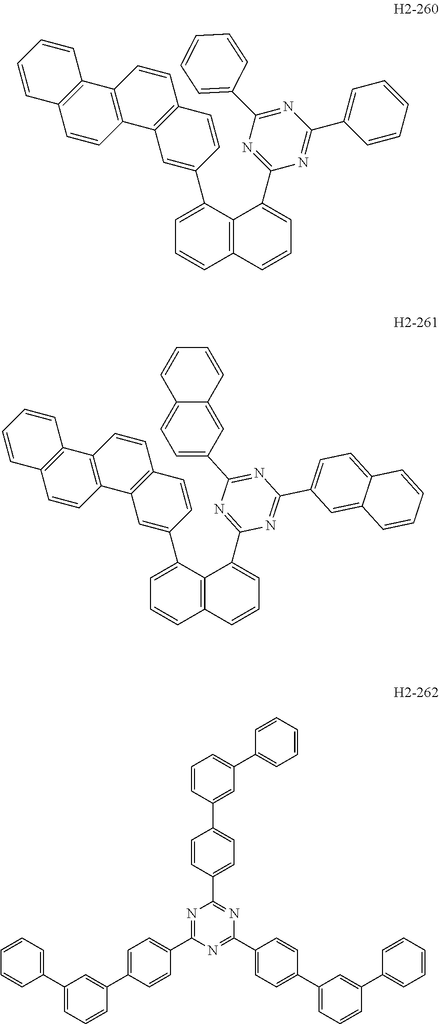 [SMILES: c1ccc(-c2nc(-c3ccccc3)nc(-c3cccc4cccc(-c5ccc6ccc7c8ccccc8ccc7c6c5)c34)n2)cc1 ; c1ccc2cc(-c3nc(-c4ccc5ccccc5c4)nc(-c4cccc5cccc(-c6ccc7ccc8c9ccccc9ccc8c7c6)c45)n3)ccc2c1 ; c1ccc(-c2cccc(-c3ccc(-c4nc(-c5ccc(-c6cccc(-c7ccccc7)c6)cc5)nc(-c5ccc(-c6cccc(-c7ccccc7)c6)cc5)n4)cc3)c2)cc1]

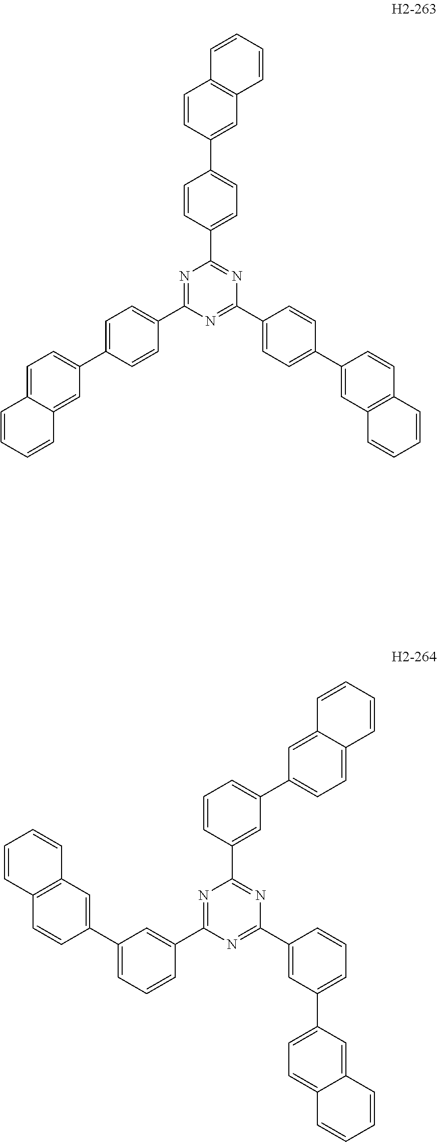 [SMILES: c1ccc2cc(-c3ccc(-c4nc(-c5ccc(-c6ccc7ccccc7c6)cc5)nc(-c5ccc(-c6ccc7ccccc7c6)cc5)n4)cc3)ccc2c1 ; c1cc(-c2ccc3ccccc3c2)cc(-c2nc(-c3cccc(-c4ccc5ccccc5c4)c3)nc(-c3cccc(-c4ccc5ccccc5c4)c3)n2)c1]

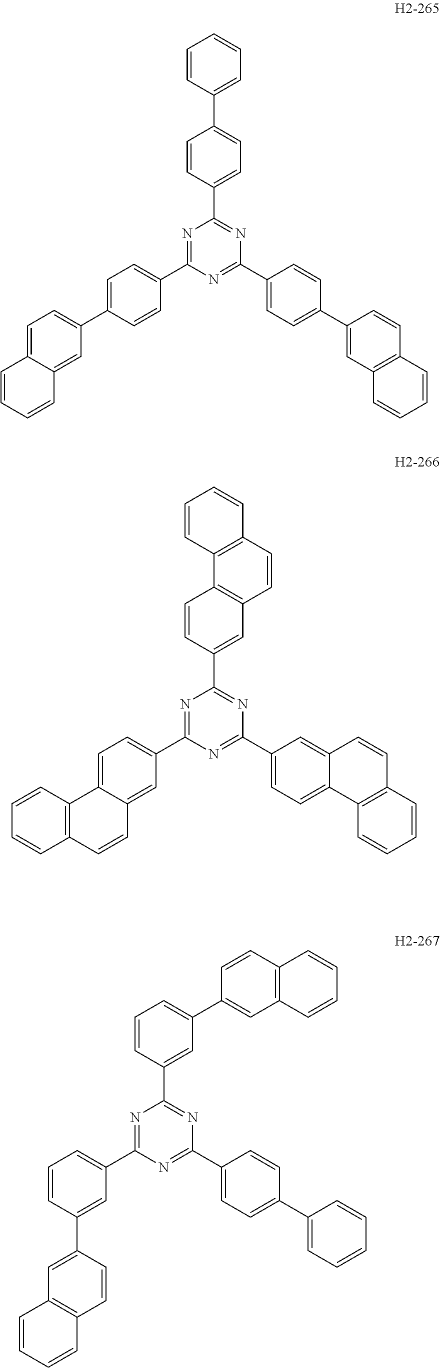 [SMILES: c1ccc(-c2ccc(-c3nc(-c4ccc(-c5ccc6ccccc6c5)cc4)nc(-c4ccc(-c5ccc6ccccc6c5)cc4)n3)cc2)cc1 ; c1ccc2c(c1)ccc1cc(-c3nc(-c4ccc5c(ccc6ccccc65)c4)nc(-c4ccc5c(ccc6ccccc65)c4)n3)ccc12 ; c1ccc(-c2ccc(-c3nc(-c4cccc(-c5ccc6ccccc6c5)c4)nc(-c4cccc(-c5ccc6ccccc6c5)c4)n3)cc2)cc1]

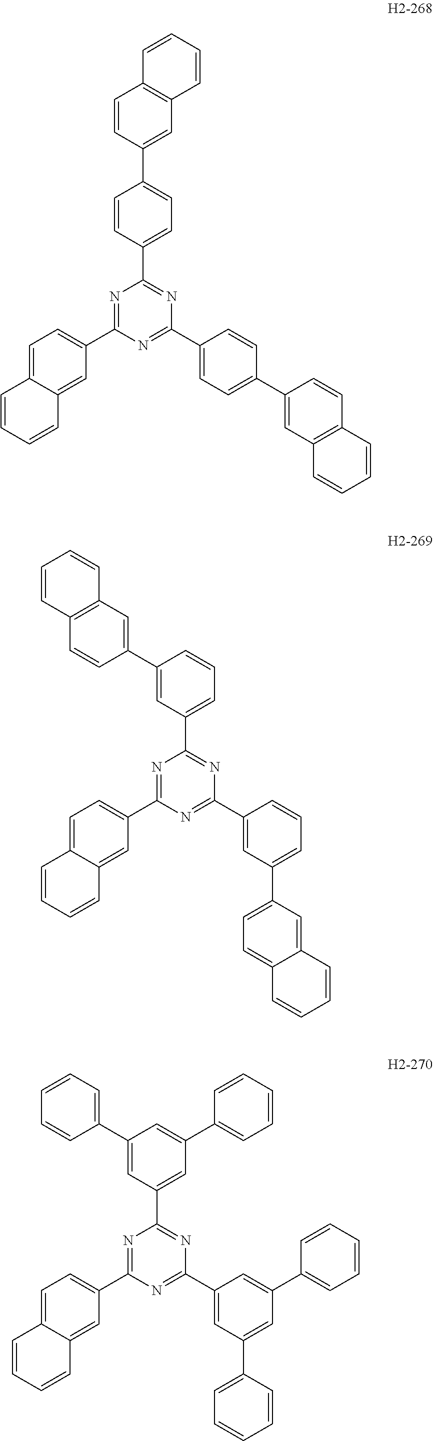 [SMILES: c1ccc2cc(-c3ccc(-c4nc(-c5ccc(-c6ccc7ccccc7c6)cc5)nc(-c5ccc6ccccc6c5)n4)cc3)ccc2c1 ; c1cc(-c2ccc3ccccc3c2)cc(-c2nc(-c3cccc(-c4ccc5ccccc5c4)c3)nc(-c3ccc4ccccc4c3)n2)c1 ; c1ccc(-c2cc(-c3ccccc3)cc(-c3nc(-c4cc(-c5ccccc5)cc(-c5ccccc5)c4)nc(-c4ccc5ccccc5c4)n3)c2)cc1]

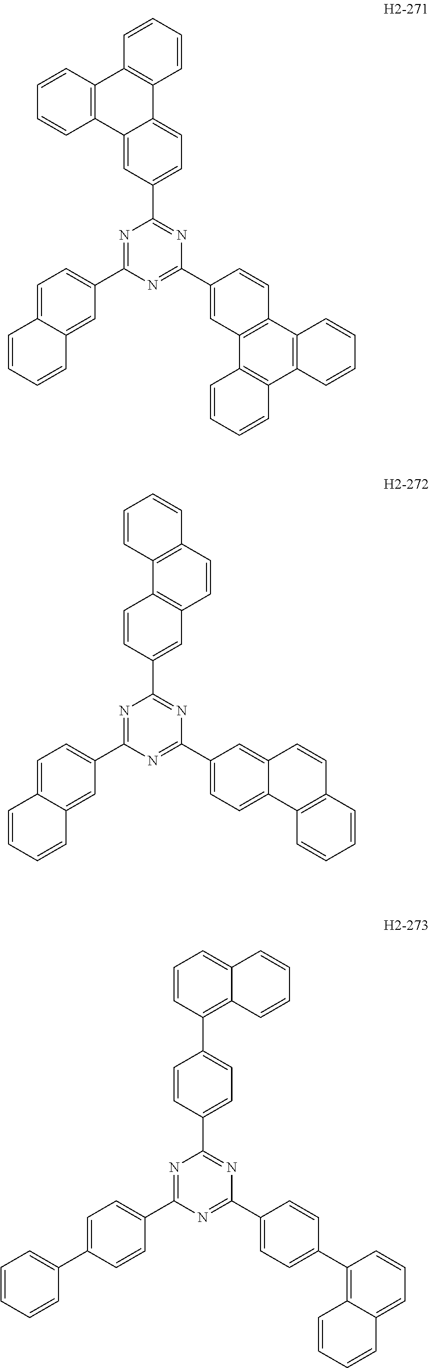 [SMILES: c1ccc2cc(-c3nc(-c4ccc5c6ccccc6c6ccccc6c5c4)nc(-c4ccc5c6ccccc6c6ccccc6c5c4)n3)ccc2c1 ; c1ccc2cc(-c3nc(-c4ccc5c(ccc6ccccc65)c4)nc(-c4ccc5c(ccc6ccccc65)c4)n3)ccc2c1 ; c1ccc(-c2ccc(-c3nc(-c4ccc(-c5cccc6ccccc56)cc4)nc(-c4ccc(-c5cccc6ccccc56)cc4)n3)cc2)cc1]

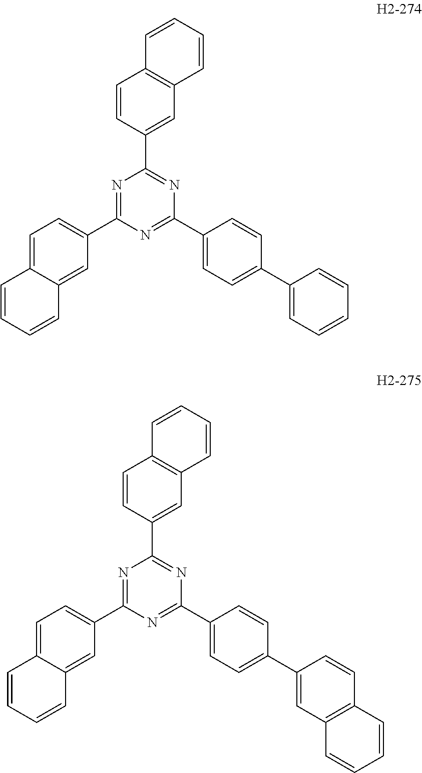 [SMILES: c1ccc(-c2ccc(-c3nc(-c4ccc5ccccc5c4)nc(-c4ccc5ccccc5c4)n3)cc2)cc1 ; c1ccc2cc(-c3ccc(-c4nc(-c5ccc6ccccc6c5)nc(-c5ccc6ccccc6c5)n4)cc3)ccc2c1]

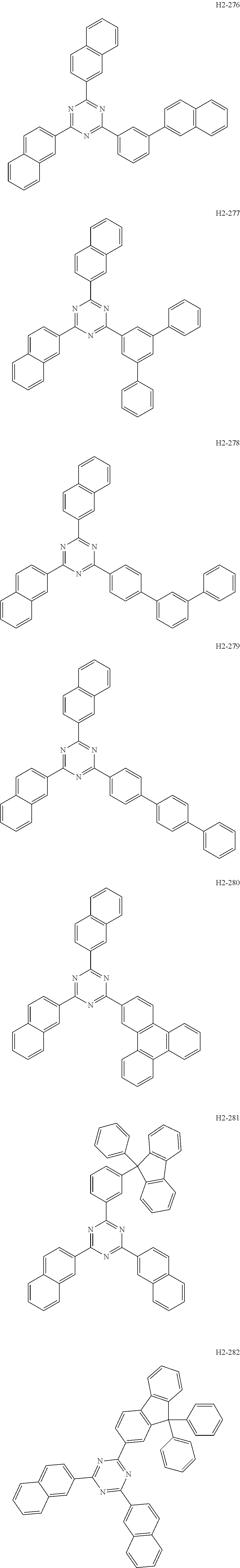

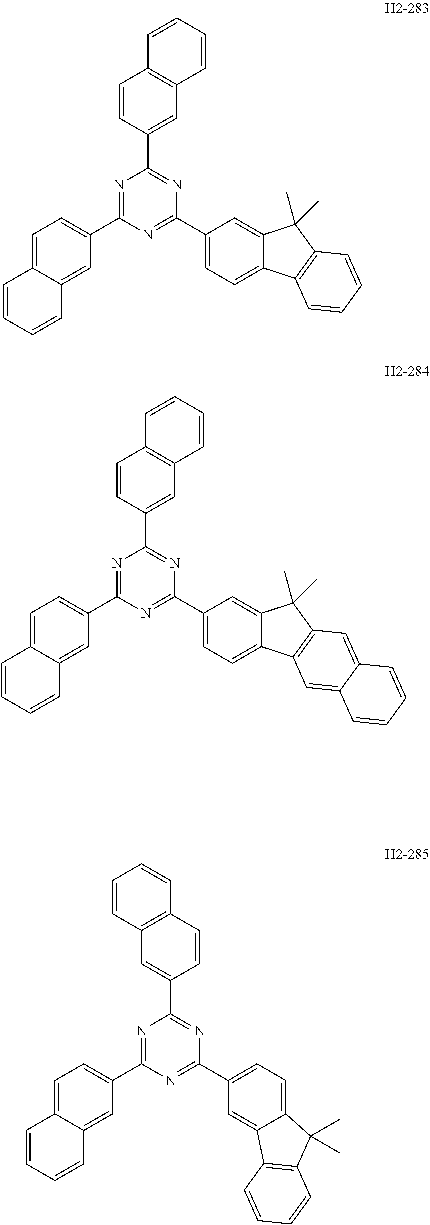 [SMILES: CC1(C)c2ccccc2-c2ccc(-c3nc(-c4ccc5ccccc5c4)nc(-c4ccc5ccccc5c4)n3)cc21 ; CC1(C)c2cc(-c3nc(-c4ccc5ccccc5c4)nc(-c4ccc5ccccc5c4)n3)ccc2-c2cc3ccccc3cc21 ; CC1(C)c2ccccc2-c2cc(-c3nc(-c4ccc5ccccc5c4)nc(-c4ccc5ccccc5c4)n3)ccc21]

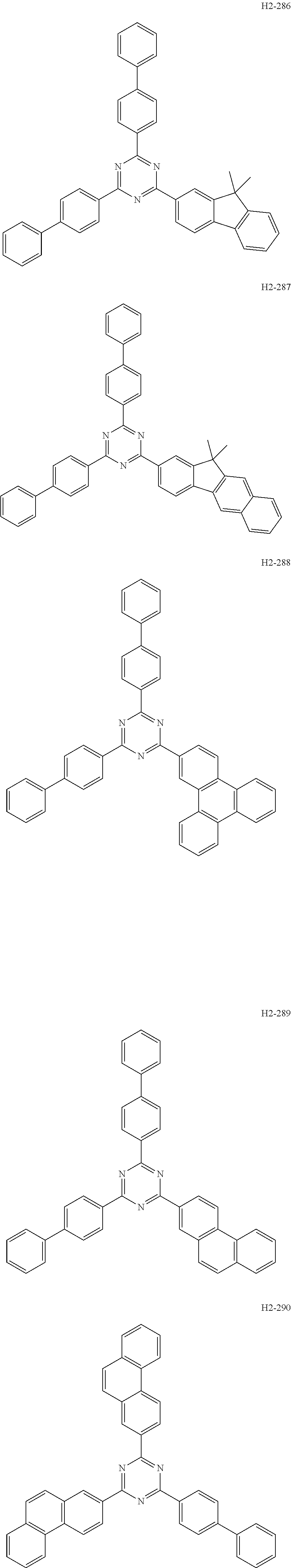 [SMILES: CC1(C)c2ccccc2-c2ccc(-c3nc(-c4ccc(-c5ccccc5)cc4)nc(-c4ccc(-c5ccccc5)cc4)n3)cc21 ; CC1(C)c2cc(-c3nc(-c4ccc(-c5ccccc5)cc4)nc(-c4ccc(-c5ccccc5)cc4)n3)ccc2-c2cc3ccccc3cc21 ; c1ccc(-c2ccc(-c3nc(-c4ccc(-c5ccccc5)cc4)nc(-c4ccc5c6ccccc6c6ccccc6c5c4)n3)cc2)cc1 ; c1ccc(-c2ccc(-c3nc(-c4ccc(-c5ccccc5)cc4)nc(-c4ccc5c(ccc6ccccc65)c4)n3)cc2)cc1 ; c1ccc(-c2ccc(-c3nc(-c4ccc5c(ccc6ccccc65)c4)nc(-c4ccc5c(ccc6ccccc65)c4)n3)cc2)cc1]

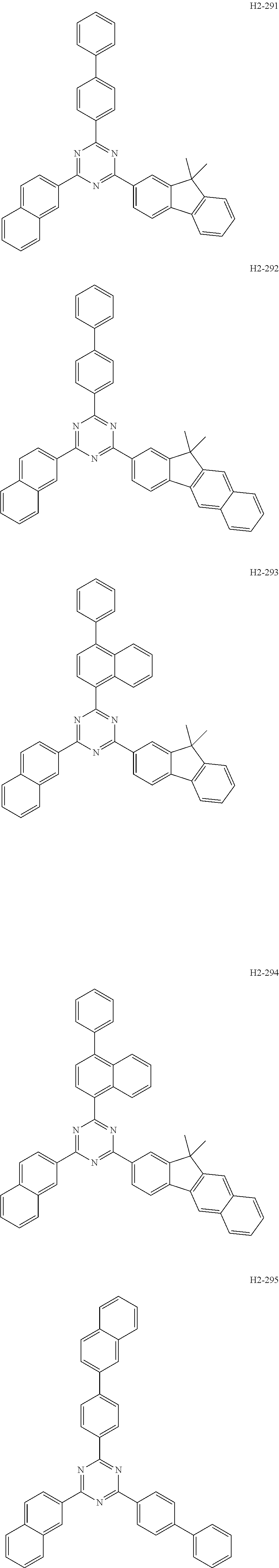 [SMILES: CC1(C)c2ccccc2-c2ccc(-c3nc(-c4ccc(-c5ccccc5)cc4)nc(-c4ccc5ccccc5c4)n3)cc21 ; CC1(C)c2cc(-c3nc(-c4ccc(-c5ccccc5)cc4)nc(-c4ccc5ccccc5c4)n3)ccc2-c2cc3ccccc3cc21 ; CC1(C)c2ccccc2-c2ccc(-c3nc(-c4ccc5ccccc5c4)nc(-c4ccc(-c5ccccc5)c5ccccc45)n3)cc21 ; CC1(C)c2cc(-c3nc(-c4ccc5ccccc5c4)nc(-c4ccc(-c5ccccc5)c5ccccc45)n3)ccc2-c2cc3ccccc3cc21 ; c1ccc(-c2ccc(-c3nc(-c4ccc(-c5ccc6ccccc6c5)cc4)nc(-c4ccc5ccccc5c4)n3)cc2)cc1]

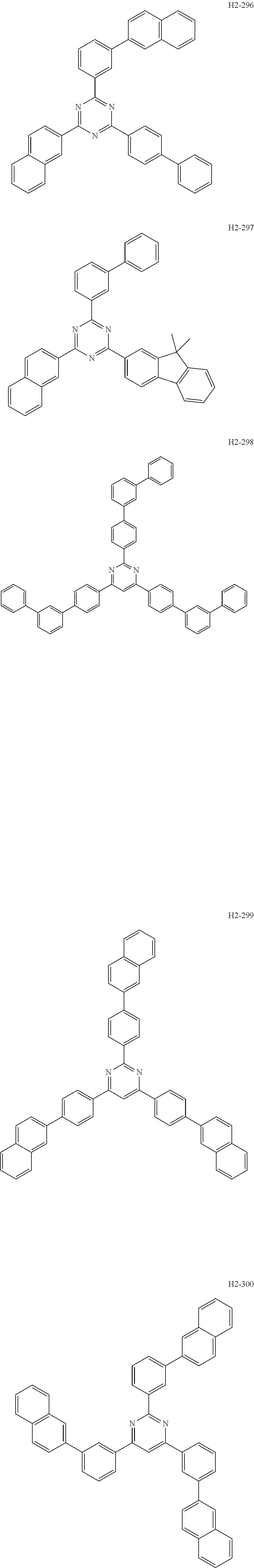 [SMILES: c1ccc(-c2ccc(-c3nc(-c4cccc(-c5ccc6ccccc6c5)c4)nc(-c4ccc5ccccc5c4)n3)cc2)cc1 ; CC1(C)c2ccccc2-c2ccc(-c3nc(-c4cccc(-c5ccccc5)c4)nc(-c4ccc5ccccc5c4)n3)cc21 ; c1ccc(-c2cccc(-c3ccc(-c4cc(-c5ccc(-c6cccc(-c7ccccc7)c6)cc5)nc(-c5ccc(-c6cccc(-c7ccccc7)c6)cc5)n4)cc3)c2)cc1 ; c1ccc2cc(-c3ccc(-c4cc(-c5ccc(-c6ccc7ccccc7c6)cc5)nc(-c5ccc(-c6ccc7ccccc7c6)cc5)n4)cc3)ccc2c1 ; c1cc(-c2ccc3ccccc3c2)cc(-c2cc(-c3cccc(-c4ccc5ccccc5c4)c3)nc(-c3cccc(-c4ccc5ccccc5c4)c3)n2)c1]

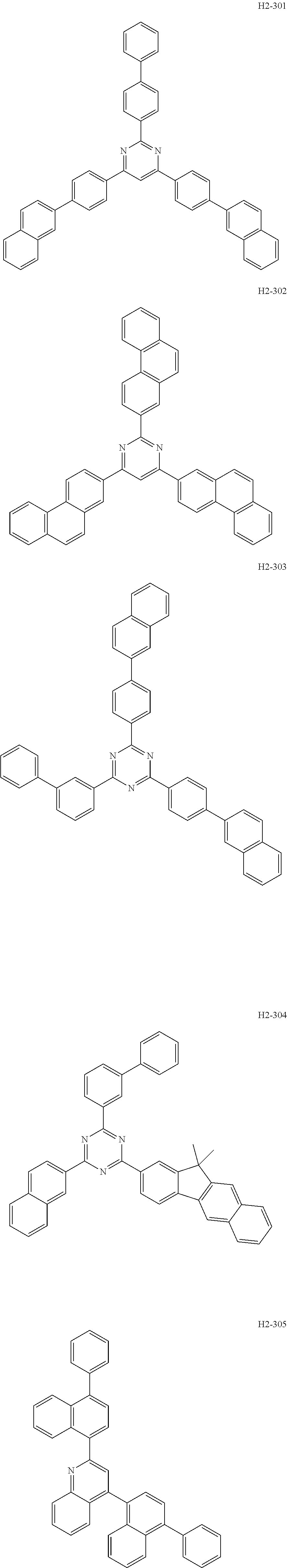 [SMILES: c1ccc(-c2ccc(-c3nc(-c4ccc(-c5ccc6ccccc6c5)cc4)cc(-c4ccc(-c5ccc6ccccc6c5)cc4)n3)cc2)cc1 ; c1ccc2c(c1)ccc1cc(-c3cc(-c4ccc5c(ccc6ccccc65)c4)nc(-c4ccc5c(ccc6ccccc65)c4)n3)ccc12 ; c1ccc(-c2cccc(-c3nc(-c4ccc(-c5ccc6ccccc6c5)cc4)nc(-c4ccc(-c5ccc6ccccc6c5)cc4)n3)c2)cc1 ; CC1(C)c2cc(-c3nc(-c4cccc(-c5ccccc5)c4)nc(-c4ccc5ccccc5c4)n3)ccc2-c2cc3ccccc3cc21 ; c1ccc(-c2ccc(-c3cc(-c4ccc(-c5ccccc5)c5ccccc45)c4ccccc4n3)c3ccccc23)cc1]

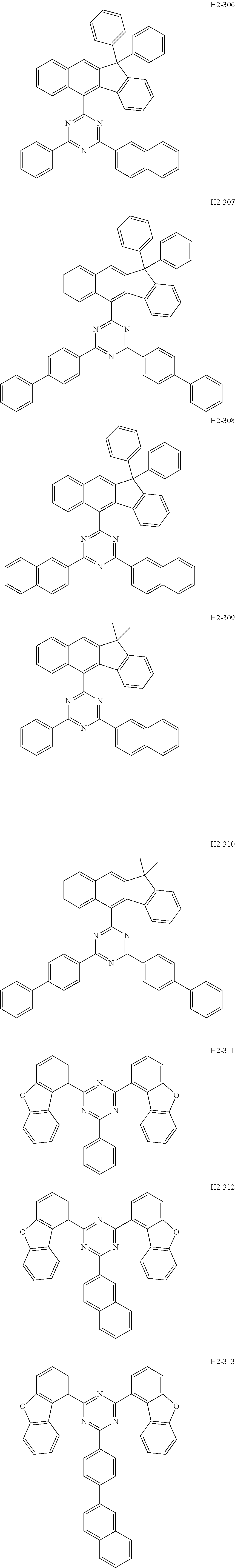

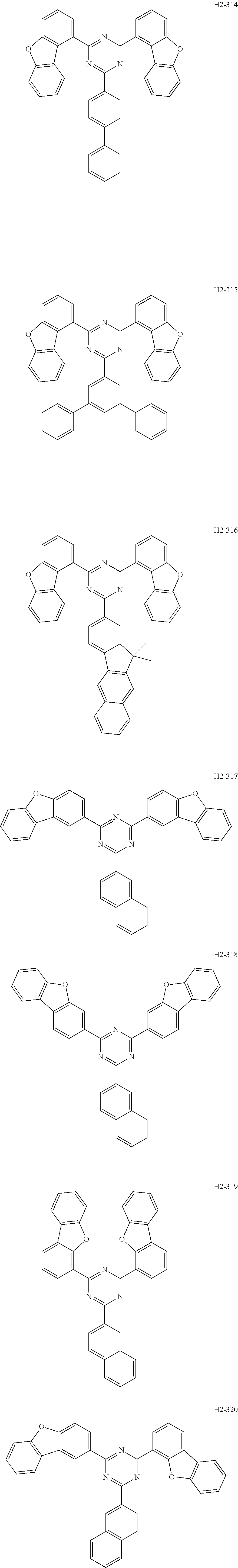

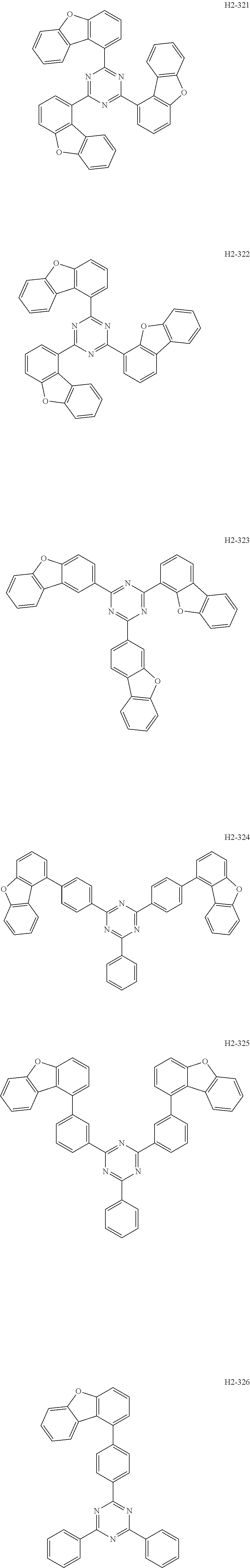 [SMILES: c1ccc2c(c1)oc1cccc(-c3nc(-c4cccc5oc6ccccc6c45)nc(-c4cccc5oc6ccccc6c45)n3)c12 ; c1ccc2c(c1)oc1c(-c3nc(-c4cccc5oc6ccccc6c45)nc(-c4cccc5oc6ccccc6c45)n3)cccc12 ; c1ccc2c(c1)oc1cc(-c3nc(-c4ccc5oc6ccccc6c5c4)nc(-c4cccc5c4oc4ccccc45)n3)ccc12 ; c1ccc(-c2nc(-c3ccc(-c4cccc5oc6ccccc6c45)cc3)nc(-c3ccc(-c4cccc5oc6ccccc6c45)cc3)n2)cc1 ; c1ccc(-c2nc(-c3cccc(-c4cccc5oc6ccccc6c45)c3)nc(-c3cccc(-c4cccc5oc6ccccc6c45)c3)n2)cc1 ; c1ccc(-c2nc(-c3ccccc3)nc(-c3ccc(-c4cccc5oc6ccccc6c45)cc3)n2)cc1]

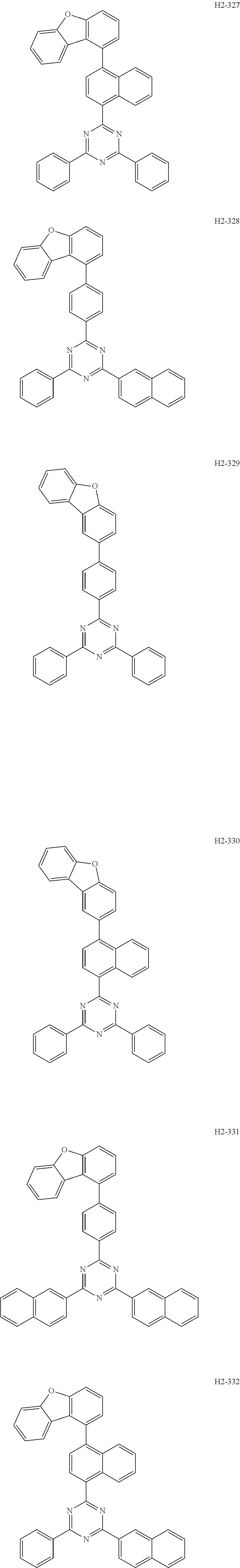 [SMILES: c1ccc(-c2nc(-c3ccccc3)nc(-c3ccc(-c4cccc5oc6ccccc6c45)c4ccccc34)n2)cc1 ; c1ccc(-c2nc(-c3ccc(-c4cccc5oc6ccccc6c45)cc3)nc(-c3ccc4ccccc4c3)n2)cc1 ; c1ccc(-c2nc(-c3ccccc3)nc(-c3ccc(-c4ccc5oc6ccccc6c5c4)cc3)n2)cc1 ; c1ccc(-c2nc(-c3ccccc3)nc(-c3ccc(-c4ccc5oc6ccccc6c5c4)c4ccccc34)n2)cc1 ; c1ccc2cc(-c3nc(-c4ccc(-c5cccc6oc7ccccc7c56)cc4)nc(-c4ccc5ccccc5c4)n3)ccc2c1 ; c1ccc(-c2nc(-c3ccc4ccccc4c3)nc(-c3ccc(-c4cccc5oc6ccccc6c45)c4ccccc34)n2)cc1]

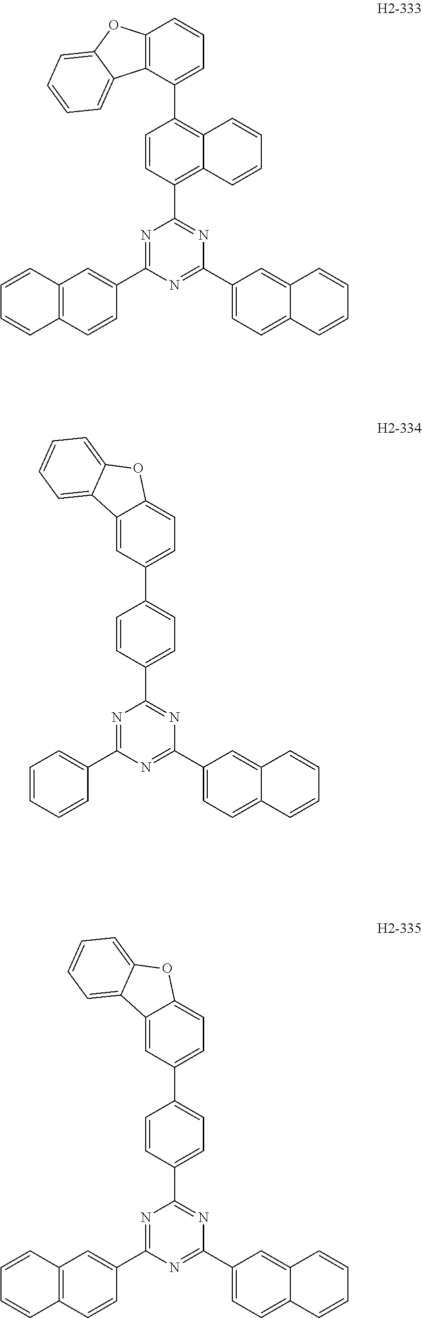 [SMILES: c1ccc2cc(-c3nc(-c4ccc5ccccc5c4)nc(-c4ccc(-c5cccc6oc7ccccc7c56)c5ccccc45)n3)ccc2c1 ; c1ccc(-c2nc(-c3ccc(-c4ccc5oc6ccccc6c5c4)cc3)nc(-c3ccc4ccccc4c3)n2)cc1 ; c1ccc2cc(-c3nc(-c4ccc(-c5ccc6oc7ccccc7c6c5)cc4)nc(-c4ccc5ccccc5c4)n3)ccc2c1]

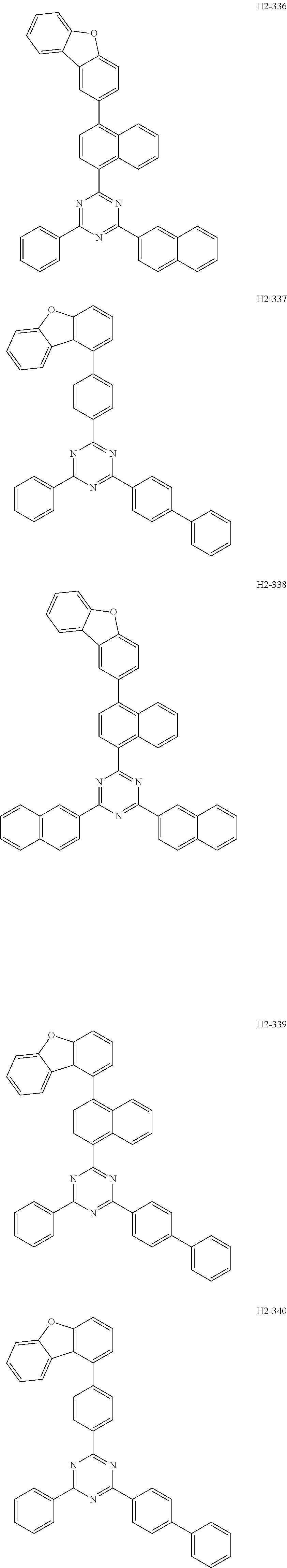 [SMILES: c1ccc(-c2nc(-c3ccc4ccccc4c3)nc(-c3ccc(-c4ccc5oc6ccccc6c5c4)c4ccccc34)n2)cc1 ; c1ccc(-c2ccc(-c3nc(-c4ccccc4)nc(-c4ccc(-c5cccc6oc7ccccc7c56)cc4)n3)cc2)cc1 ; c1ccc2cc(-c3nc(-c4ccc5ccccc5c4)nc(-c4ccc(-c5ccc6oc7ccccc7c6c5)c5ccccc45)n3)ccc2c1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2ccc(-c3nc(-c4ccccc4)nc(-c4ccc(-c5cccc6oc7ccccc7c56)cc4)n3)cc2)cc1]

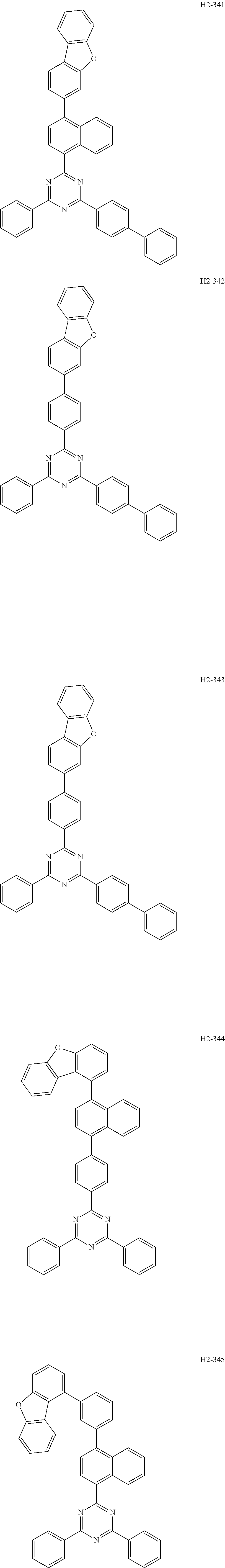 [SMILES: c1ccc(-c2ccc(-c3nc(-c4ccccc4)nc(-c4ccc(-c5ccc6c(c5)oc5ccccc56)c5ccccc45)n3)cc2)cc1 ; c1ccc(-c2ccc(-c3nc(-c4ccccc4)nc(-c4ccc(-c5ccc6c(c5)oc5ccccc56)cc4)n3)cc2)cc1 ; c1ccc(-c2ccc(-c3nc(-c4ccccc4)nc(-c4ccc(-c5ccc6c(c5)oc5ccccc56)cc4)n3)cc2)cc1 ; c1ccc(-c2nc(-c3ccccc3)nc(-c3ccc(-c4ccc(-c5cccc6oc7ccccc7c56)c5ccccc45)cc3)n2)cc1 ; c1ccc(-c2nc(-c3ccccc3)nc(-c3ccc(-c4cccc(-c5cccc6oc7ccccc7c56)c4)c4ccccc34)n2)cc1]

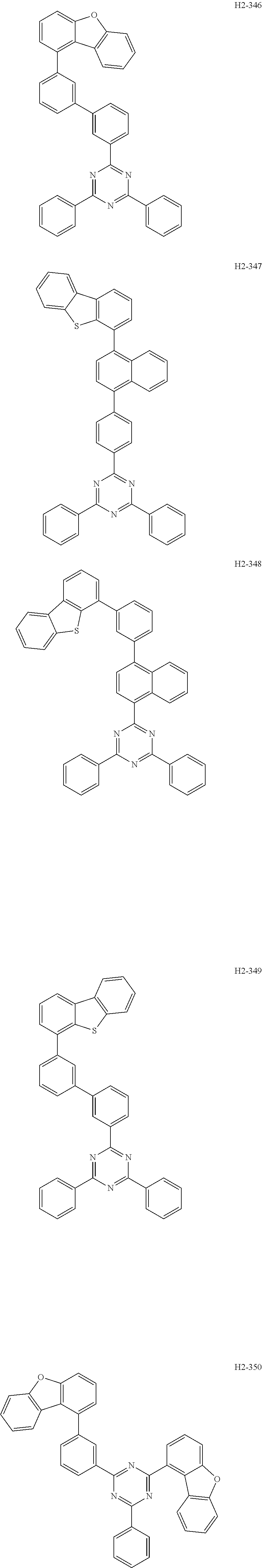 [SMILES: c1ccc(-c2nc(-c3ccccc3)nc(-c3cccc(-c4cccc(-c5cccc6oc7ccccc7c56)c4)c3)n2)cc1 ; c1ccc(-c2nc(-c3ccccc3)nc(-c3ccc(-c4ccc(-c5cccc6c5sc5ccccc56)c5ccccc45)cc3)n2)cc1 ; c1ccc(-c2nc(-c3ccccc3)nc(-c3ccc(-c4cccc(-c5cccc6c5sc5ccccc56)c4)c4ccccc34)n2)cc1 ; c1ccc(-c2nc(-c3ccccc3)nc(-c3cccc(-c4cccc(-c5cccc6c5sc5ccccc56)c4)c3)n2)cc1 ; c1ccc(-c2nc(-c3cccc(-c4cccc5oc6ccccc6c45)c3)nc(-c3cccc4oc5ccccc5c34)n2)cc1]

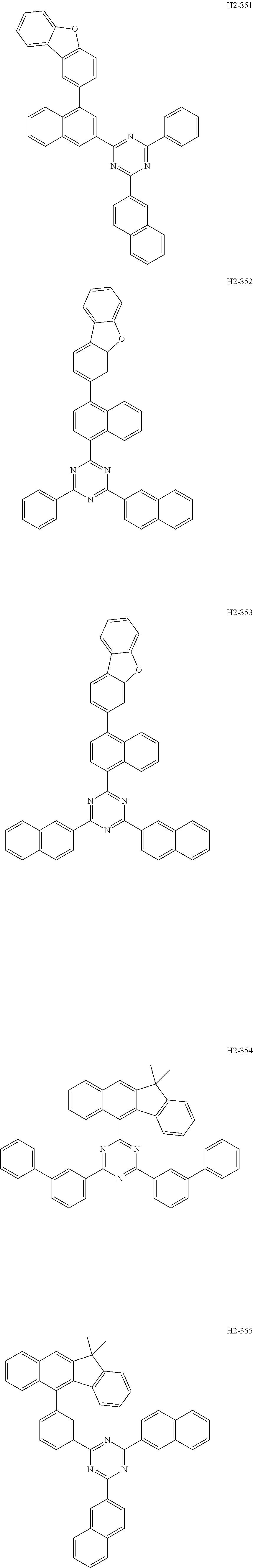 [SMILES: c1ccc(-c2nc(-c3ccc4ccccc4c3)nc(-c3cc(-c4ccc5oc6ccccc6c5c4)c4ccccc4c3)n2)cc1 ; c1ccc(-c2nc(-c3ccc4ccccc4c3)nc(-c3ccc(-c4ccc5c(c4)oc4ccccc45)c4ccccc34)n2)cc1 ; c1ccc2cc(-c3nc(-c4ccc5ccccc5c4)nc(-c4ccc(-c5ccc6c(c5)oc5ccccc56)c5ccccc45)n3)ccc2c1 ; CC1(C)c2ccccc2-c2c1cc1ccccc1c2-c1nc(-c2cccc(-c3ccccc3)c2)nc(-c2cccc(-c3ccccc3)c2)n1 ; CC1(C)c2ccccc2-c2c1cc1ccccc1c2-c1cccc(-c2nc(-c3ccc4ccccc4c3)nc(-c3ccc4ccccc4c3)n2)c1]

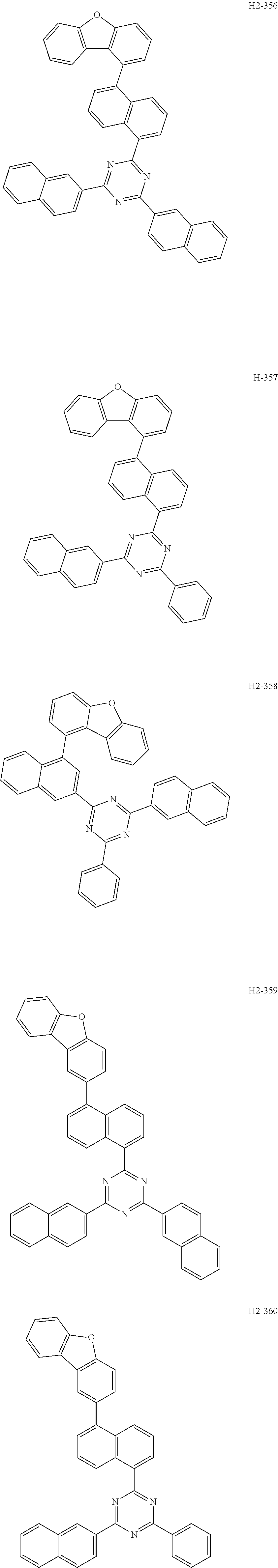 [SMILES: c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1 ; c1ccc2cc(-c3nc(-c4ccc5ccccc5c4)nc(-c4cccc5c(-c6ccc7oc8ccccc8c7c6)cccc45)n3)ccc2c1 ; c1ccc(-c2nc(-c3ccc4ccccc4c3)nc(-c3cccc4c(-c5ccc6oc7ccccc7c6c5)cccc34)n2)cc1]

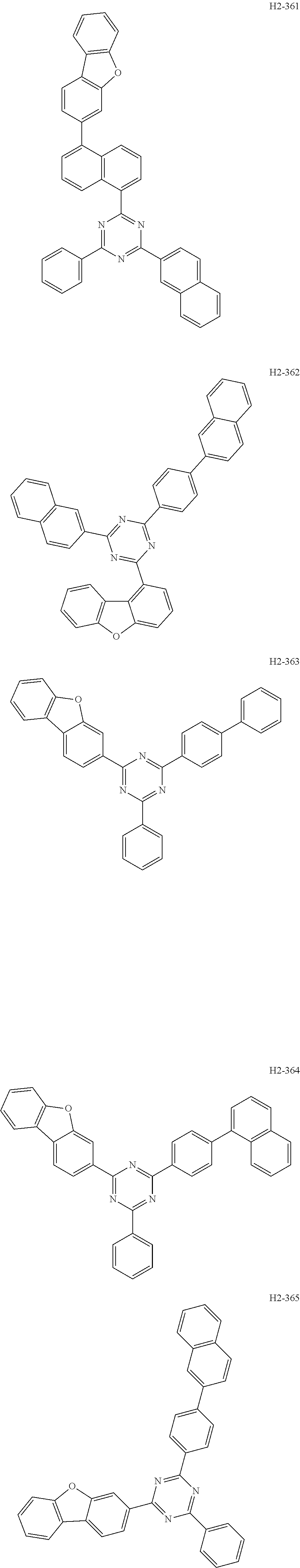 [SMILES: c1ccc(-c2nc(-c3ccc4ccccc4c3)nc(-c3cccc4c(-c5ccc6c(c5)oc5ccccc56)cccc34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccccc4)nc(-c4ccc5c(c4)oc4ccccc45)n3)cc2)cc1 ; c1ccc(-c2nc(-c3ccc(-c4cccc5ccccc45)cc3)nc(-c3ccc4c(c3)oc3ccccc34)n2)cc1 ; c1ccc(-c2nc(-c3ccc(-c4ccc5ccccc5c4)cc3)nc(-c3ccc4c(c3)oc3ccccc34)n2)cc1]

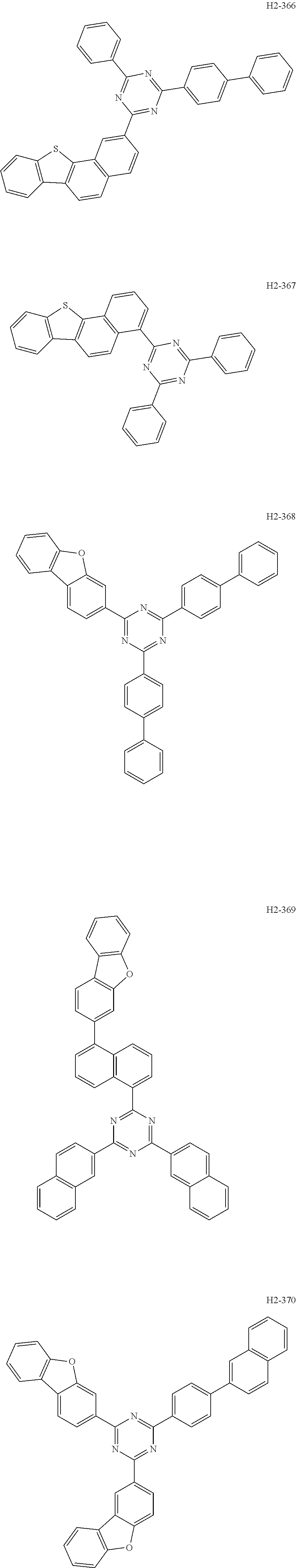 [SMILES: c1ccc(-c2ccc(-c3nc(-c4ccccc4)nc(-c4ccc5ccc6c7ccccc7sc6c5c4)n3)cc2)cc1 ; c1ccc(-c2nc(-c3ccccc3)nc(-c3cccc4c3ccc3c5ccccc5sc43)n2)cc1 ; c1ccc(-c2ccc(-c3nc(-c4ccc(-c5ccccc5)cc4)nc(-c4ccc5c(c4)oc4ccccc45)n3)cc2)cc1 ; c1ccc2cc(-c3nc(-c4ccc5ccccc5c4)nc(-c4cccc5c(-c6ccc7c(c6)oc6ccccc67)cccc45)n3)ccc2c1 ; c1ccc2cc(-c3ccc(-c4nc(-c5ccc6c(c5)oc5ccccc56)nc(-c5ccc6oc7ccccc7c6c5)n4)cc3)ccc2c1]

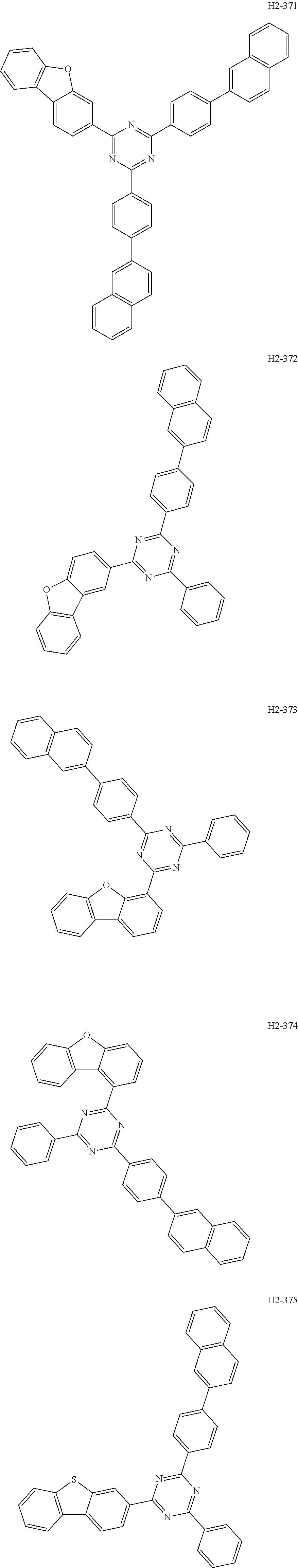 [SMILES: c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1 ; c1ccc(-c2nc(-c3ccc(-c4ccc5ccccc5c4)cc3)nc(-c3cccc4c3oc3ccccc34)n2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc4c(c3)sc3ccccc34)n2)cc1]

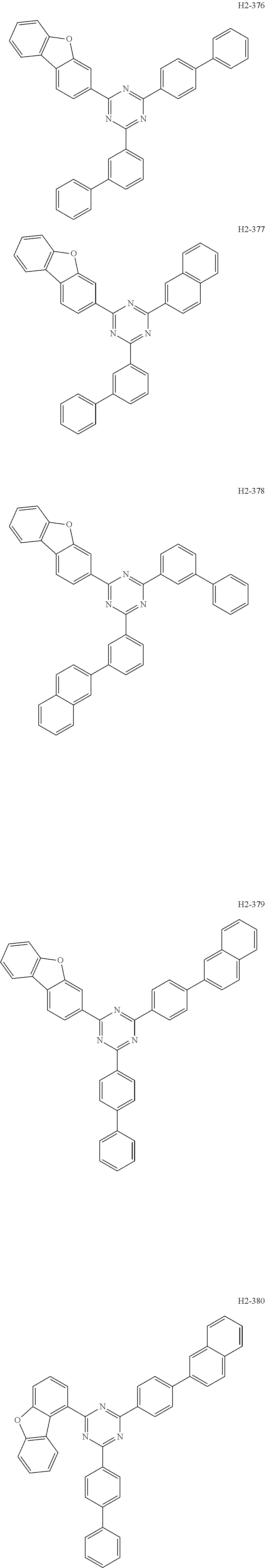 [SMILES: c1ccc(-c2ccc(-c3nc(-c4cccc(-c5ccccc5)c4)nc(-c4ccc5c(c4)oc4ccccc45)n3)cc2)cc1 ; c1ccc(-c2cccc(-c3nc(-c4ccc5ccccc5c4)nc(-c4ccc5c(c4)oc4ccccc45)n3)c2)cc1 ; c1ccc(-c2cccc(-c3nc(-c4cccc(-c5ccc6ccccc6c5)c4)nc(-c4ccc5c(c4)oc4ccccc45)n3)c2)cc1 ; c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2ccc(-c3nc(-c4ccc(-c5ccc6ccccc6c5)cc4)nc(-c4cccc5oc6ccccc6c45)n3)cc2)cc1]

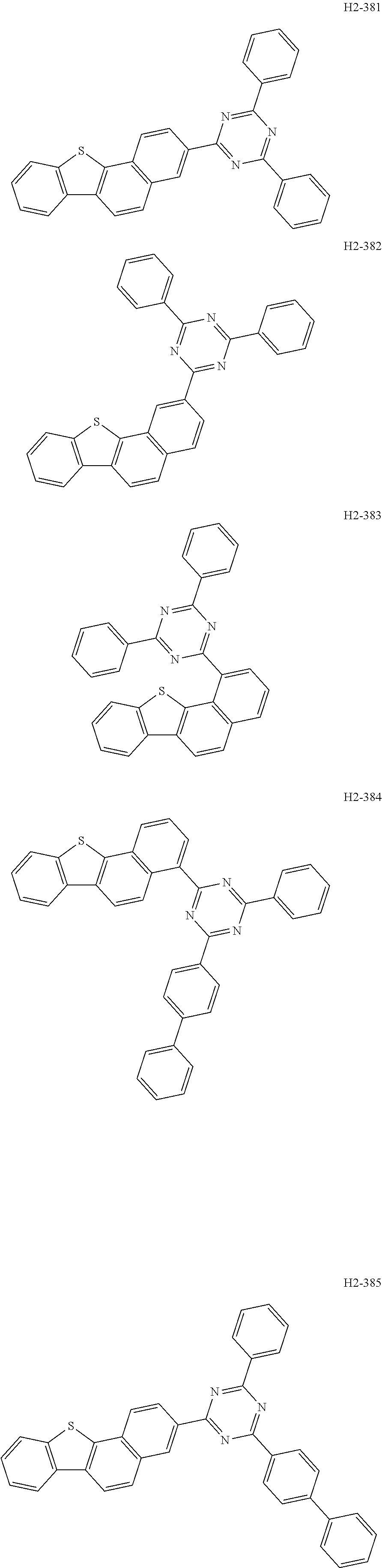 [SMILES: c1ccc(-c2nc(-c3ccccc3)nc(-c3ccc4c(ccc5c6ccccc6sc45)c3)n2)cc1 ; c1ccc(-c2nc(-c3ccccc3)nc(-c3ccc4ccc5c6ccccc6sc5c4c3)n2)cc1 ; c1ccc(-c2nc(-c3ccccc3)nc(-c3cccc4ccc5c6ccccc6sc5c34)n2)cc1 ; c1ccc(-c2ccc(-c3nc(-c4ccccc4)nc(-c4cccc5c4ccc4c6ccccc6sc54)n3)cc2)cc1 ; c1ccc(-c2ccc(-c3nc(-c4ccccc4)nc(-c4ccc5c(ccc6c7ccccc7sc56)c4)n3)cc2)cc1]

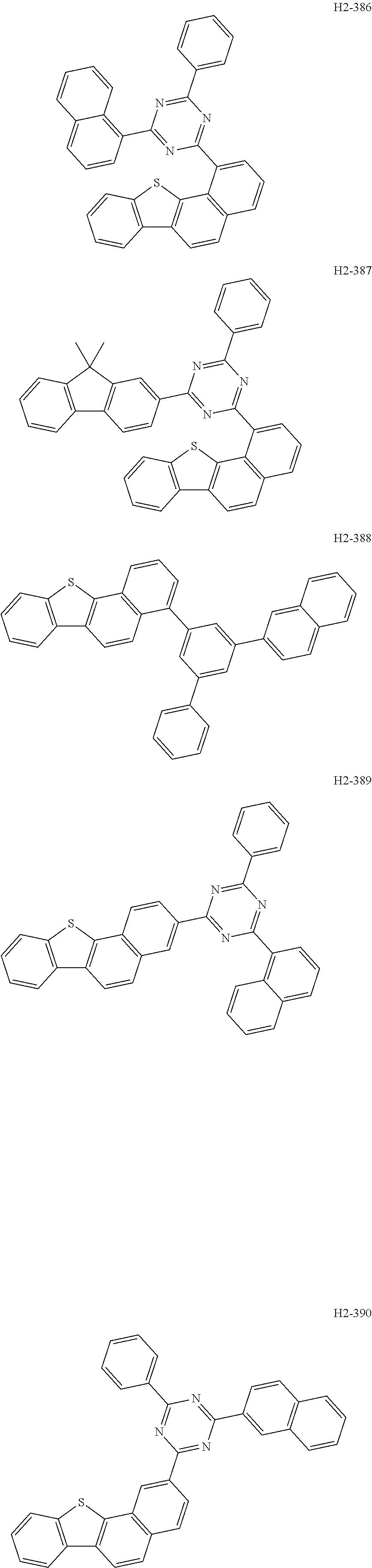 [SMILES: c1ccc(-c2nc(-c3cccc4ccccc34)nc(-c3cccc4ccc5c6ccccc6sc5c34)n2)cc1 ; CC1(C)c2ccccc2-c2ccc(-c3nc(-c4ccccc4)nc(-c4cccc5ccc6c7ccccc7sc6c45)n3)cc21 ; c1ccc(-c2cc(-c3ccc4ccccc4c3)cc(-c3cccc4c3ccc3c5ccccc5sc43)c2)cc1 ; c1ccc(-c2nc(-c3ccc4c(ccc5c6ccccc6sc45)c3)nc(-c3cccc4ccccc34)n2)cc1 ; c1ccc(-c2nc(-c3ccc4ccccc4c3)nc(-c3ccc4ccc5c6ccccc6sc5c4c3)n2)cc1]

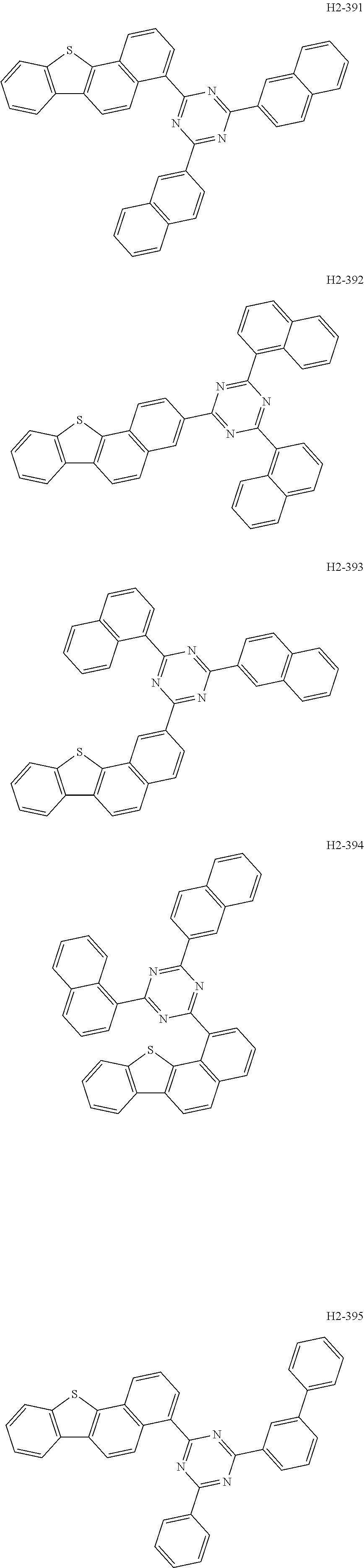 [SMILES: c1ccc2cc(-c3nc(-c4ccc5ccccc5c4)nc(-c4cccc5c4ccc4c6ccccc6sc54)n3)ccc2c1 ; c1ccc2c(-c3nc(-c4ccc5c(ccc6c7ccccc7sc56)c4)nc(-c4cccc5ccccc45)n3)cccc2c1 ; c1ccc2cc(-c3nc(-c4ccc5ccc6c7ccccc7sc6c5c4)nc(-c4cccc5ccccc45)n3)ccc2c1 ; c1ccc2cc(-c3nc(-c4cccc5ccccc45)nc(-c4cccc5ccc6c7ccccc7sc6c45)n3)ccc2c1 ; c1ccc(-c2cccc(-c3nc(-c4ccccc4)nc(-c4cccc5c4ccc4c6ccccc6sc54)n3)c2)cc1]

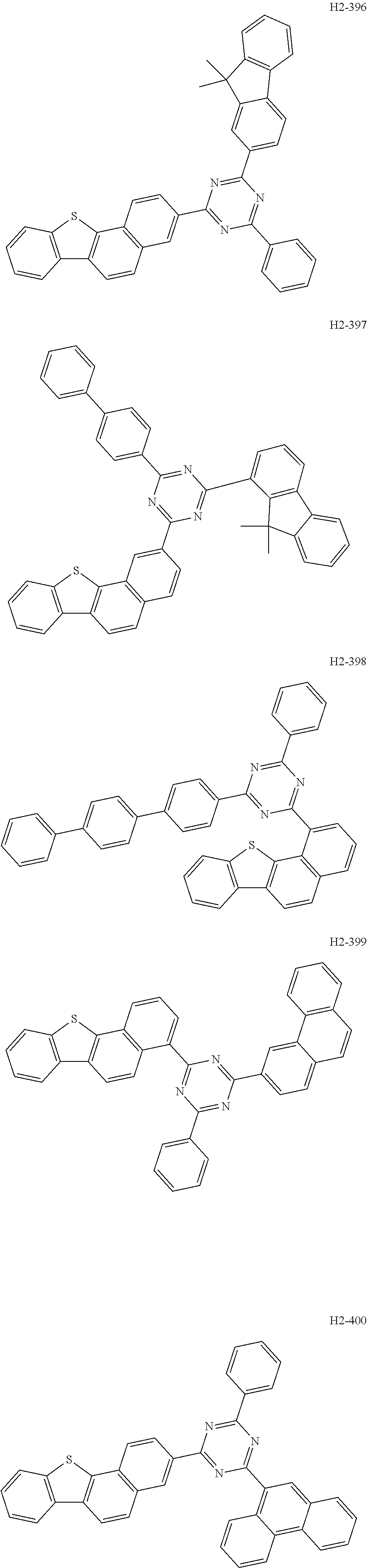 [SMILES: CC1(C)c2ccccc2-c2ccc(-c3nc(-c4ccccc4)nc(-c4ccc5c(ccc6c7ccccc7sc56)c4)n3)cc21 ; CC1(C)c2ccccc2-c2cccc(-c3nc(-c4ccc(-c5ccccc5)cc4)nc(-c4ccc5ccc6c7ccccc7sc6c5c4)n3)c21 ; c1ccc(-c2ccc(-c3ccc(-c4nc(-c5ccccc5)nc(-c5cccc6ccc7c8ccccc8sc7c56)n4)cc3)cc2)cc1 ; c1ccc(-c2nc(-c3ccc4ccc5ccccc5c4c3)nc(-c3cccc4c3ccc3c5ccccc5sc43)n2)cc1 ; c1ccc(-c2nc(-c3ccc4c(ccc5c6ccccc6sc45)c3)nc(-c3cc4ccccc4c4ccccc34)n2)cc1]

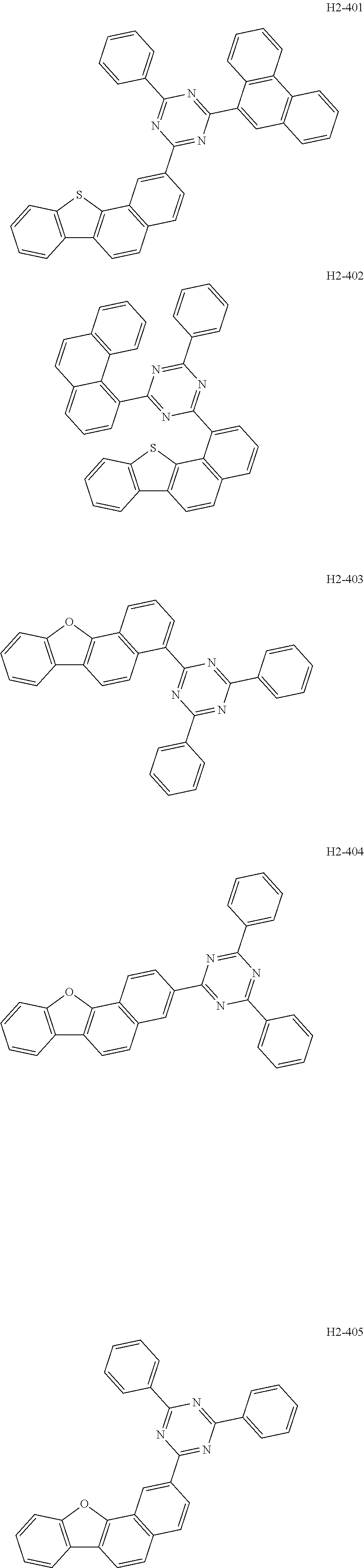 [SMILES: c1ccc(-c2nc(-c3ccc4ccc5c6ccccc6sc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6sc5c34)n2)cc1 ; c1ccc(-c2nc(-c3ccccc3)nc(-c3cccc4c3ccc3c5ccccc5oc43)n2)cc1 ; c1ccc(-c2nc(-c3ccccc3)nc(-c3ccc4c(ccc5c6ccccc6oc45)c3)n2)cc1 ; c1ccc(-c2nc(-c3ccccc3)nc(-c3ccc4ccc5c6ccccc6oc5c4c3)n2)cc1]

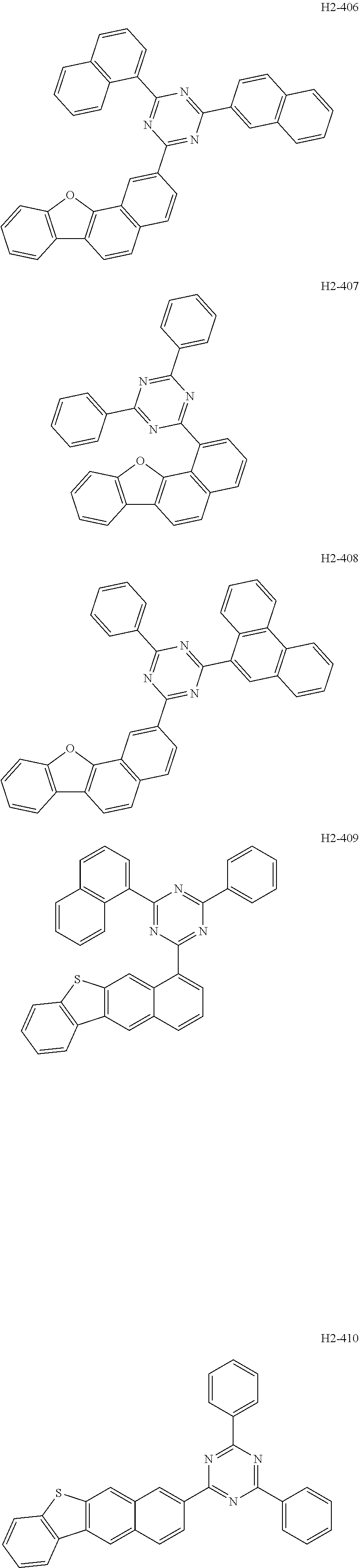

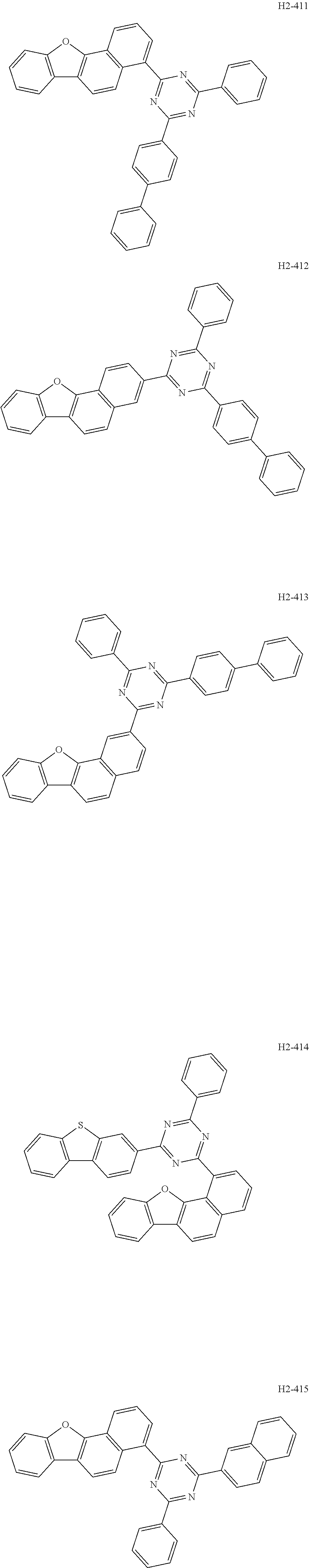 [SMILES: c1ccc(-c2ccc(-c3nc(-c4ccccc4)nc(-c4cccc5c4ccc4c6ccccc6oc54)n3)cc2)cc1 ; c1ccc(-c2ccc(-c3nc(-c4ccccc4)nc(-c4ccc5c(ccc6c7ccccc7oc56)c4)n3)cc2)cc1 ; c1ccc(-c2ccc(-c3nc(-c4ccccc4)nc(-c4ccc5ccc6c7ccccc7oc6c5c4)n3)cc2)cc1 ; c1ccc(-c2nc(-c3ccc4c(c3)sc3ccccc34)nc(-c3cccc4ccc5c6ccccc6oc5c34)n2)cc1 ; c1ccc(-c2nc(-c3ccc4ccccc4c3)nc(-c3cccc4c3ccc3c5ccccc5oc43)n2)cc1]

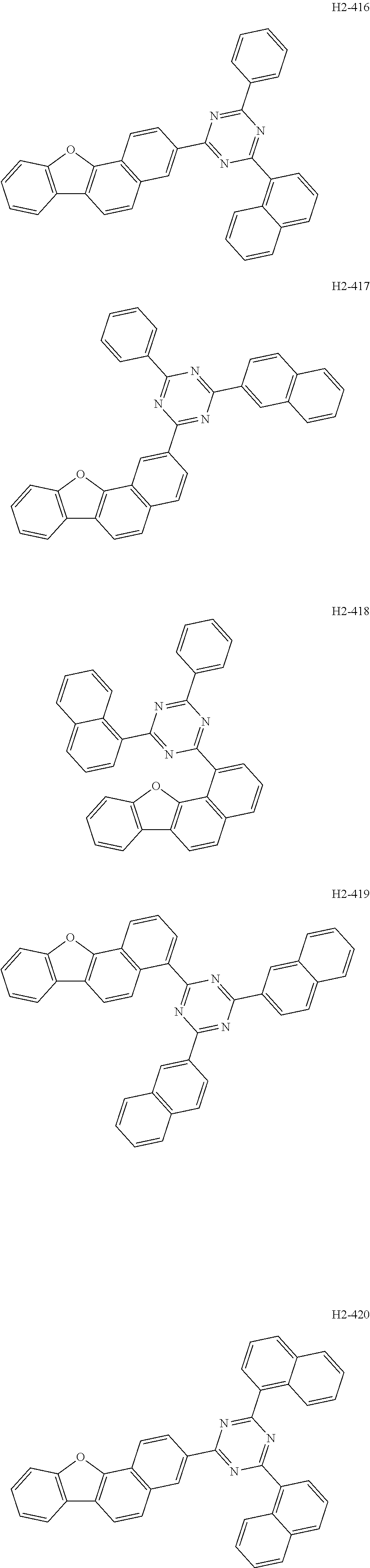 [SMILES: c1ccc(-c2nc(-c3ccc4c(ccc5c6ccccc6oc45)c3)nc(-c3cccc4ccccc34)n2)cc1 ; c1ccc(-c2nc(-c3ccc4ccccc4c3)nc(-c3ccc4ccc5c6ccccc6oc5c4c3)n2)cc1 ; c1ccc(-c2nc(-c3cccc4ccccc34)nc(-c3cccc4ccc5c6ccccc6oc5c34)n2)cc1 ; c1ccc2cc(-c3nc(-c4ccc5ccccc5c4)nc(-c4cccc5c4ccc4c6ccccc6oc54)n3)ccc2c1 ; c1ccc2c(-c3nc(-c4ccc5c(ccc6c7ccccc7oc56)c4)nc(-c4cccc5ccccc45)n3)cccc2c1]

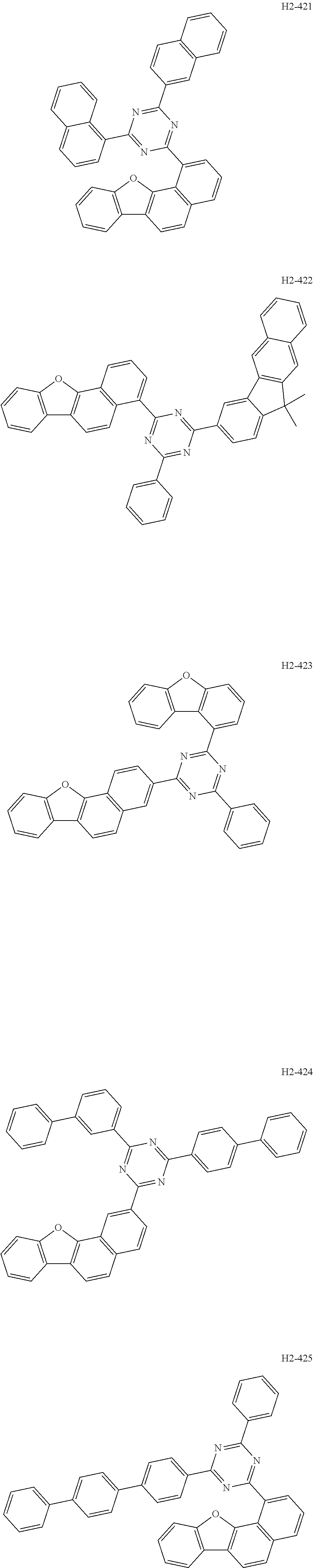 [SMILES: c1ccc2cc(-c3nc(-c4cccc5ccccc45)nc(-c4cccc5ccc6c7ccccc7oc6c45)n3)ccc2c1 ; CC1(C)c2ccc(-c3nc(-c4ccccc4)nc(-c4cccc5c4ccc4c6ccccc6oc54)n3)cc2-c2cc3ccccc3cc21 ; c1ccc(-c2nc(-c3ccc4c(ccc5c6ccccc6oc45)c3)nc(-c3cccc4oc5ccccc5c34)n2)cc1 ; c1ccc(-c2ccc(-c3nc(-c4cccc(-c5ccccc5)c4)nc(-c4ccc5ccc6c7ccccc7oc6c5c4)n3)cc2)cc1 ; c1ccc(-c2ccc(-c3ccc(-c4nc(-c5ccccc5)nc(-c5cccc6ccc7c8ccccc8oc7c56)n4)cc3)cc2)cc1]

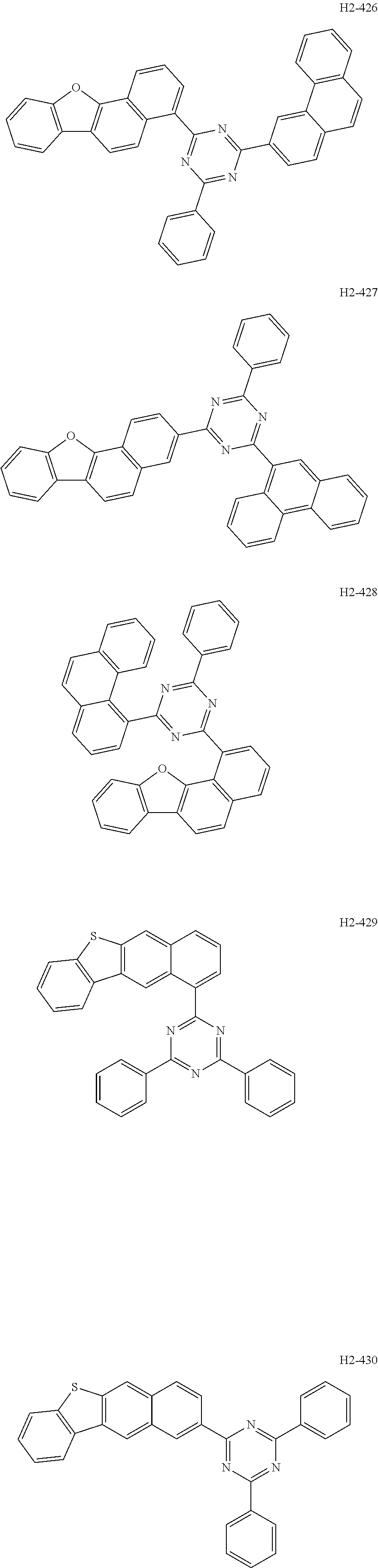 [SMILES: c1ccc(-c2nc(-c3ccc4ccc5ccccc5c4c3)nc(-c3cccc4c3ccc3c5ccccc5oc43)n2)cc1 ; c1ccc(-c2nc(-c3ccc4c(ccc5c6ccccc6oc45)c3)nc(-c3cc4ccccc4c4ccccc34)n2)cc1 ; c1ccc(-c2nc(-c3cccc4ccc5ccccc5c34)nc(-c3cccc4ccc5c6ccccc6oc5c34)n2)cc1 ; c1ccc(-c2nc(-c3ccccc3)nc(-c3cccc4cc5sc6ccccc6c5cc34)n2)cc1 ; c1ccc(-c2nc(-c3ccccc3)nc(-c3ccc4cc5sc6ccccc6c5cc4c3)n2)cc1]

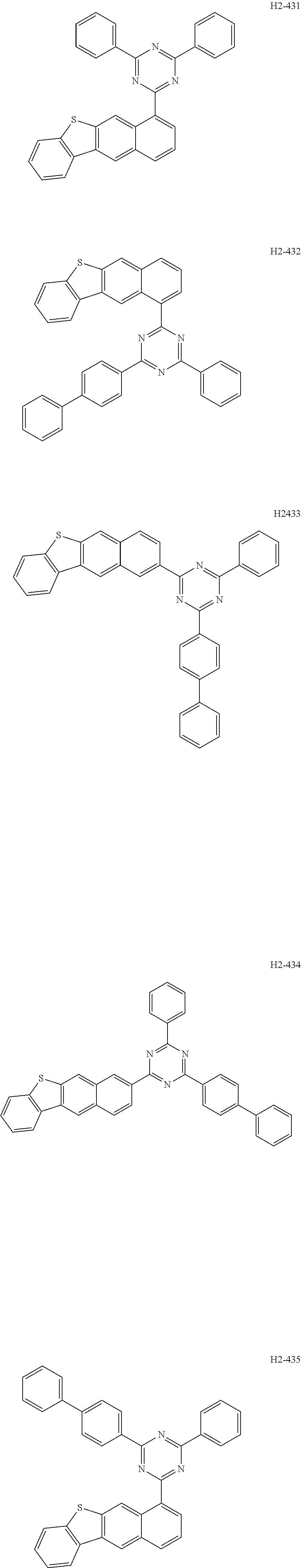 [SMILES: c1ccc(-c2nc(-c3ccccc3)nc(-c3cccc4cc5c(cc34)sc3ccccc35)n2)cc1 ; c1ccc(-c2ccc(-c3nc(-c4ccccc4)nc(-c4cccc5cc6sc7ccccc7c6cc45)n3)cc2)cc1 ; c1ccc(-c2ccc(-c3nc(-c4ccccc4)nc(-c4ccc5cc6sc7ccccc7c6cc5c4)n3)cc2)cc1 ; c1ccc(-c2ccc(-c3nc(-c4ccccc4)nc(-c4ccc5cc6c(cc5c4)sc4ccccc46)n3)cc2)cc1 ; c1ccc(-c2ccc(-c3nc(-c4ccccc4)nc(-c4cccc5cc6c(cc45)sc4ccccc46)n3)cc2)cc1]

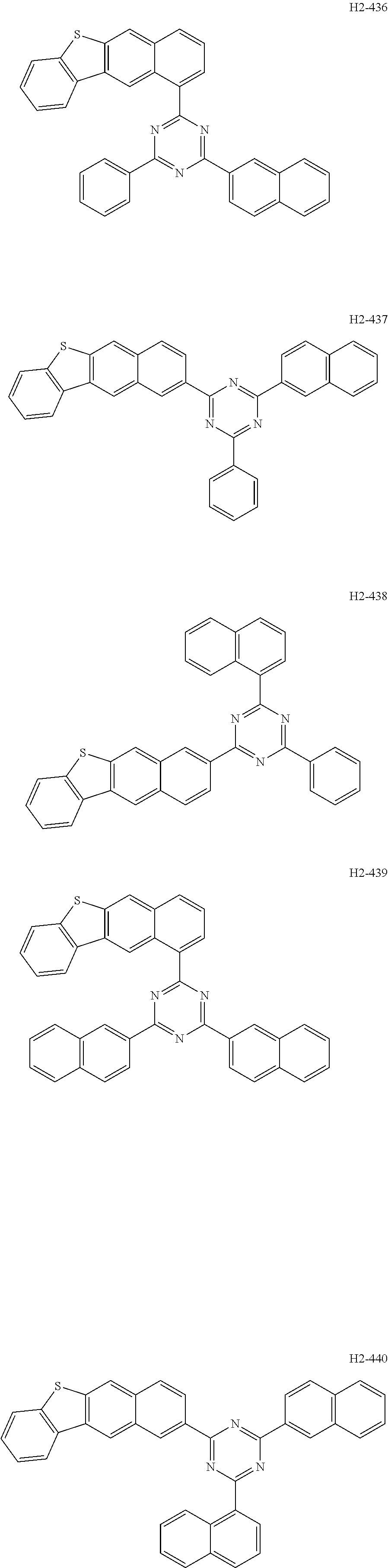 [SMILES: c1ccc(-c2nc(-c3ccc4ccccc4c3)nc(-c3cccc4cc5sc6ccccc6c5cc34)n2)cc1 ; c1ccc(-c2nc(-c3ccc4ccccc4c3)nc(-c3ccc4cc5sc6ccccc6c5cc4c3)n2)cc1 ; c1ccc(-c2nc(-c3ccc4cc5c(cc4c3)sc3ccccc35)nc(-c3cccc4ccccc34)n2)cc1 ; c1ccc2cc(-c3nc(-c4ccc5ccccc5c4)nc(-c4cccc5cc6sc7ccccc7c6cc45)n3)ccc2c1 ; c1ccc2cc(-c3nc(-c4ccc5cc6sc7ccccc7c6cc5c4)nc(-c4cccc5ccccc45)n3)ccc2c1]

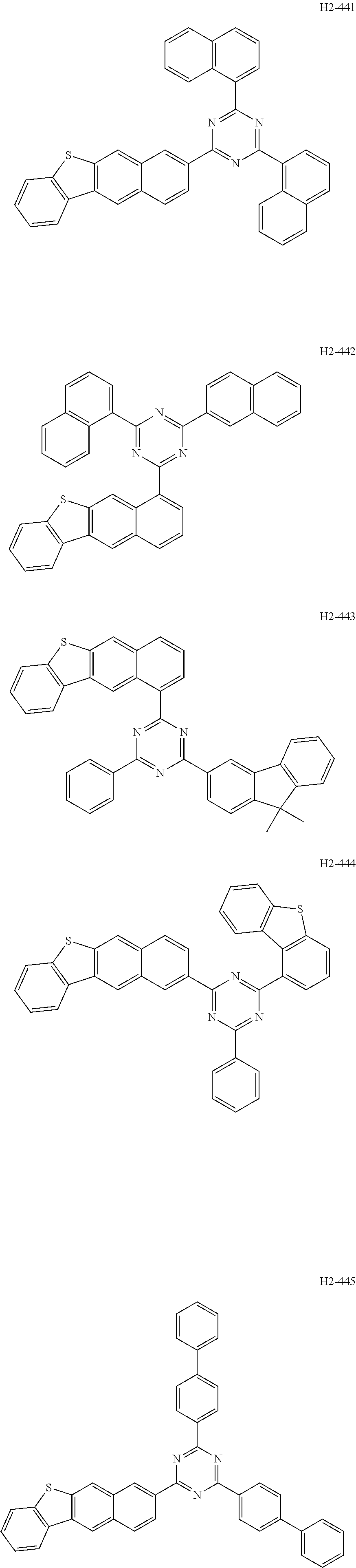 [SMILES: c1ccc2c(-c3nc(-c4ccc5cc6c(cc5c4)sc4ccccc46)nc(-c4cccc5ccccc45)n3)cccc2c1 ; c1ccc2cc(-c3nc(-c4cccc5ccccc45)nc(-c4cccc5cc6c(cc45)sc4ccccc46)n3)ccc2c1 ; CC1(C)c2ccccc2-c2cc(-c3nc(-c4ccccc4)nc(-c4cccc5cc6sc7ccccc7c6cc45)n3)ccc21 ; c1ccc(-c2nc(-c3ccc4cc5sc6ccccc6c5cc4c3)nc(-c3cccc4sc5ccccc5c34)n2)cc1 ; c1ccc(-c2ccc(-c3nc(-c4ccc(-c5ccccc5)cc4)nc(-c4ccc5cc6c(cc5c4)sc4ccccc46)n3)cc2)cc1]

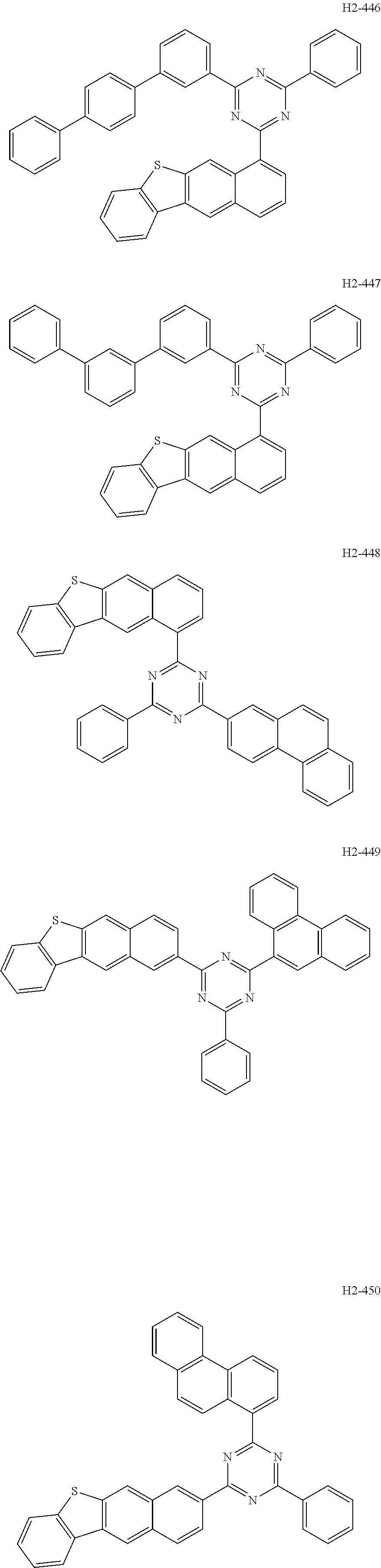 [SMILES: c1ccc(-c2ccc(-c3cccc(-c4nc(-c5ccccc5)nc(-c5cccc6cc7c(cc56)sc5ccccc57)n4)c3)cc2)cc1 ; c1ccc(-c2cccc(-c3cccc(-c4nc(-c5ccccc5)nc(-c5cccc6cc7c(cc56)sc5ccccc57)n4)c3)c2)cc1 ; c1ccc(-c2nc(-c3ccc4c(ccc5ccccc54)c3)nc(-c3cccc4cc5sc6ccccc6c5cc34)n2)cc1 ; c1ccc(-c2nc(-c3ccc4cc5sc6ccccc6c5cc4c3)nc(-c3cc4ccccc4c4ccccc34)n2)cc1 ; c1ccc(-c2nc(-c3ccc4cc5c(cc4c3)sc3ccccc35)nc(-c3cccc4c3ccc3ccccc34)n2)cc1]

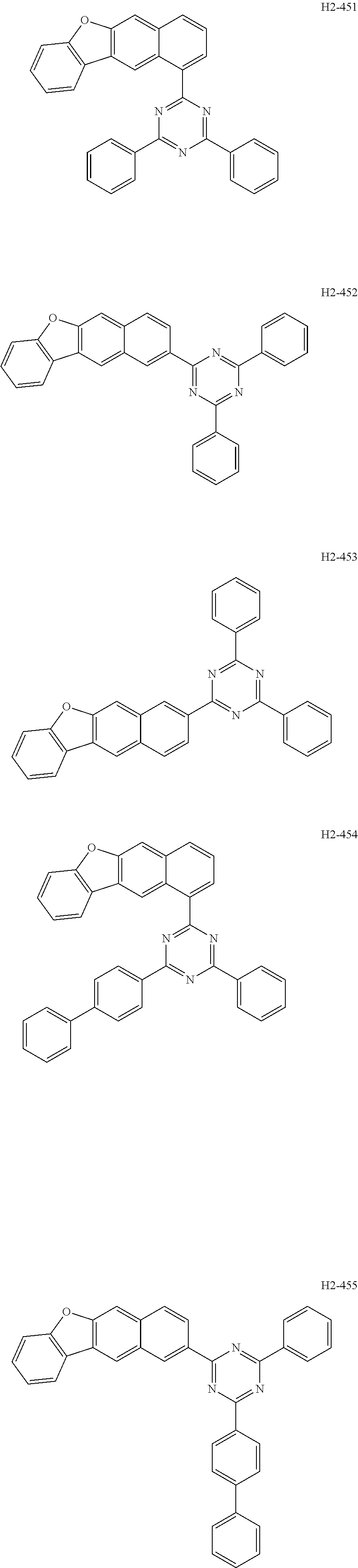 [SMILES: c1ccc(-c2nc(-c3ccccc3)nc(-c3cccc4cc5oc6ccccc6c5cc34)n2)cc1 ; c1ccc(-c2nc(-c3ccccc3)nc(-c3ccc4cc5oc6ccccc6c5cc4c3)n2)cc1 ; c1ccc(-c2nc(-c3ccccc3)nc(-c3ccc4cc5c(cc4c3)oc3ccccc35)n2)cc1 ; c1ccc(-c2ccc(-c3nc(-c4ccccc4)nc(-c4cccc5cc6oc7ccccc7c6cc45)n3)cc2)cc1 ; c1ccc(-c2ccc(-c3nc(-c4ccccc4)nc(-c4ccc5cc6oc7ccccc7c6cc5c4)n3)cc2)cc1]

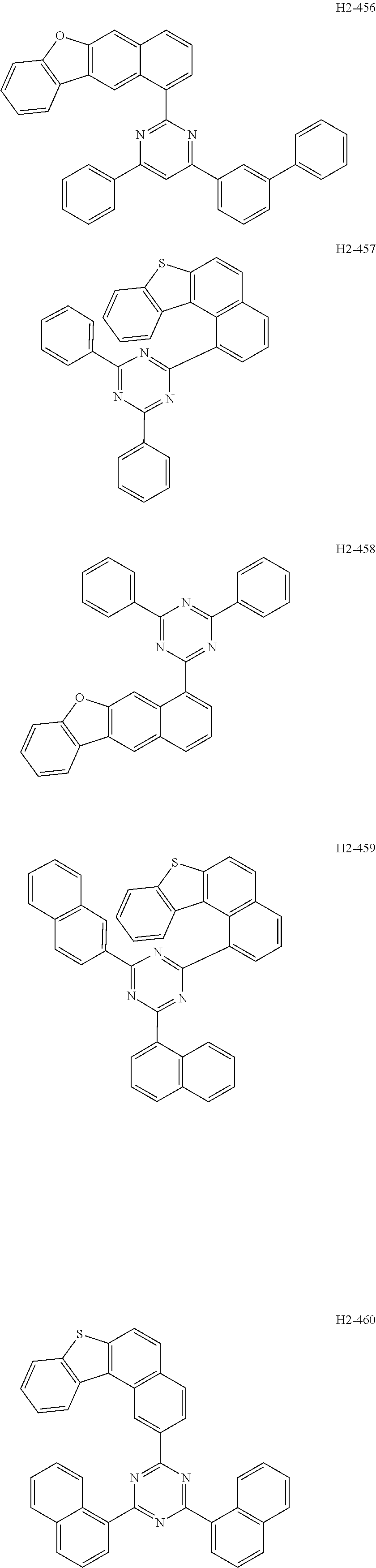 [SMILES: c1ccc(-c2cccc(-c3cc(-c4ccccc4)nc(-c4cccc5cc6oc7ccccc7c6cc45)n3)c2)cc1 ; c1ccc(-c2nc(-c3ccccc3)nc(-c3cccc4ccc5sc6ccccc6c5c34)n2)cc1 ; c1ccc(-c2nc(-c3ccccc3)nc(-c3cccc4cc5c(cc34)oc3ccccc35)n2)cc1 ; c1ccc2cc(-c3nc(-c4cccc5ccccc45)nc(-c4cccc5ccc6sc7ccccc7c6c45)n3)ccc2c1 ; c1ccc2c(-c3nc(-c4ccc5ccc6sc7ccccc7c6c5c4)nc(-c4cccc5ccccc45)n3)cccc2c1]

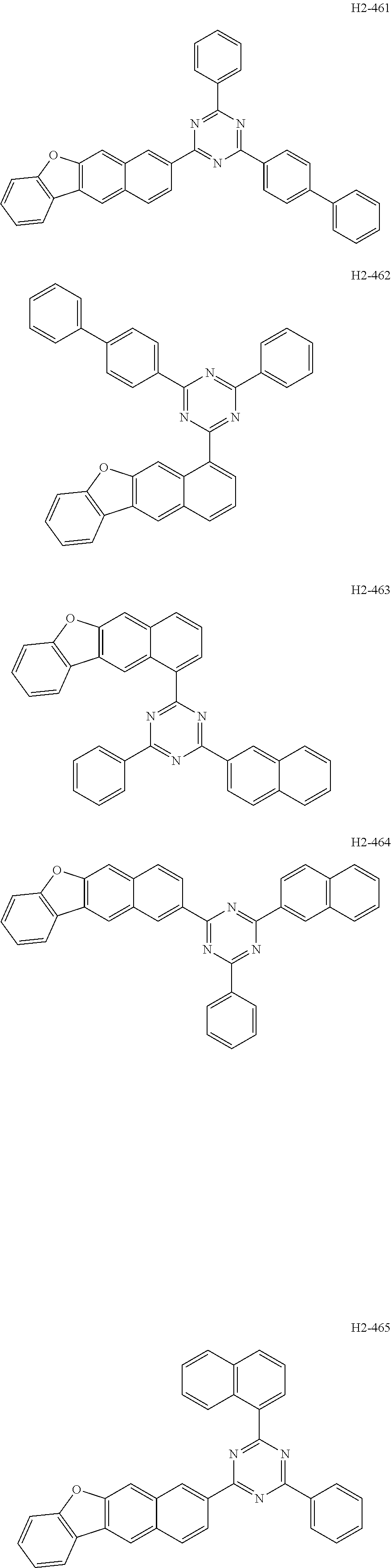 [SMILES: c1ccc(-c2ccc(-c3nc(-c4ccccc4)nc(-c4ccc5cc6c(cc5c4)oc4ccccc46)n3)cc2)cc1 ; c1ccc(-c2ccc(-c3nc(-c4ccccc4)nc(-c4cccc5cc6c(cc45)oc4ccccc46)n3)cc2)cc1 ; c1ccc(-c2nc(-c3ccc4ccccc4c3)nc(-c3cccc4cc5oc6ccccc6c5cc34)n2)cc1 ; c1ccc(-c2nc(-c3ccc4ccccc4c3)nc(-c3ccc4cc5oc6ccccc6c5cc4c3)n2)cc1 ; c1ccc(-c2nc(-c3ccc4cc5c(cc4c3)oc3ccccc35)nc(-c3cccc4ccccc34)n2)cc1]

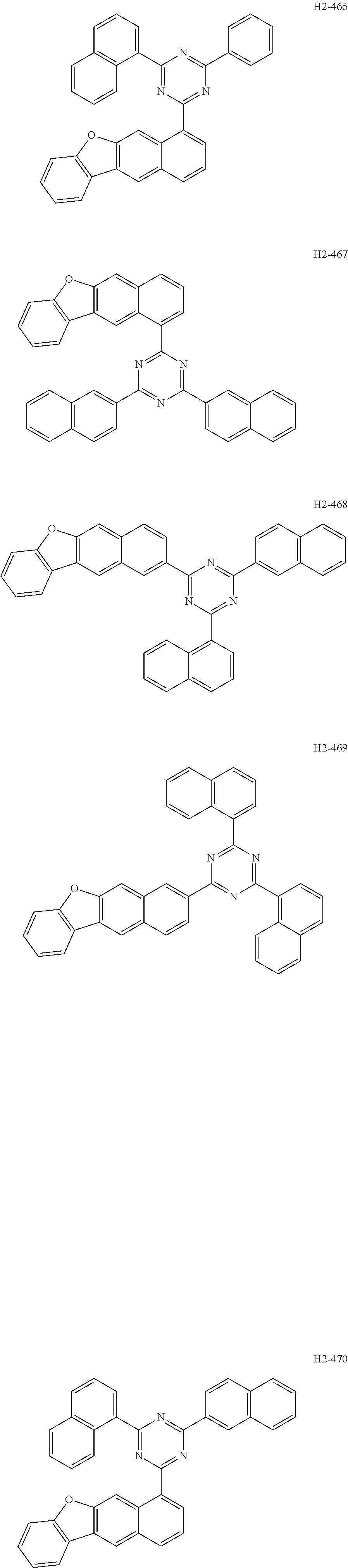 [SMILES: c1ccc(-c2nc(-c3cccc4ccccc34)nc(-c3cccc4cc5c(cc34)oc3ccccc35)n2)cc1 ; c1ccc2cc(-c3nc(-c4ccc5ccccc5c4)nc(-c4cccc5cc6oc7ccccc7c6cc45)n3)ccc2c1 ; c1ccc2cc(-c3nc(-c4ccc5cc6oc7ccccc7c6cc5c4)nc(-c4cccc5ccccc45)n3)ccc2c1 ; c1ccc2c(-c3nc(-c4ccc5cc6c(cc5c4)oc4ccccc46)nc(-c4cccc5ccccc45)n3)cccc2c1 ; c1ccc2cc(-c3nc(-c4cccc5ccccc45)nc(-c4cccc5cc6c(cc45)oc4ccccc46)n3)ccc2c1]

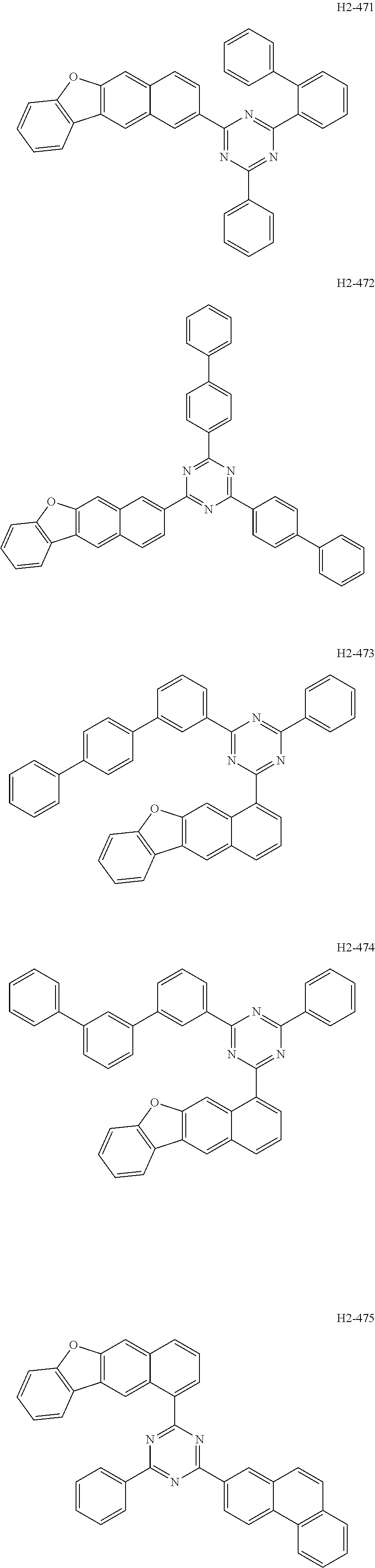 [SMILES: c1ccc(-c2nc(-c3ccc4cc5oc6ccccc6c5cc4c3)nc(-c3ccccc3-c3ccccc3)n2)cc1 ; c1ccc(-c2ccc(-c3nc(-c4ccc(-c5ccccc5)cc4)nc(-c4ccc5cc6c(cc5c4)oc4ccccc46)n3)cc2)cc1 ; c1ccc(-c2ccc(-c3cccc(-c4nc(-c5ccccc5)nc(-c5cccc6cc7c(cc56)oc5ccccc57)n4)c3)cc2)cc1 ; c1ccc(-c2cccc(-c3cccc(-c4nc(-c5ccccc5)nc(-c5cccc6cc7c(cc56)oc5ccccc57)n4)c3)c2)cc1 ; c1ccc(-c2nc(-c3ccc4c(ccc5ccccc54)c3)nc(-c3cccc4cc5oc6ccccc6c5cc34)n2)cc1]

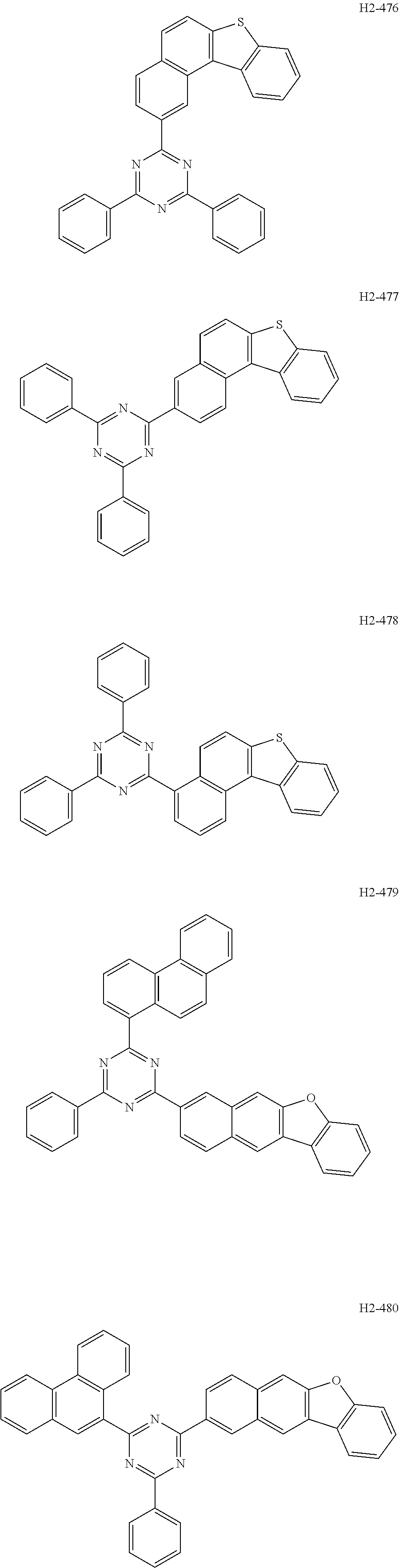 [SMILES: c1ccc(-c2nc(-c3ccccc3)nc(-c3ccc4ccc5sc6ccccc6c5c4c3)n2)cc1 ; c1ccc(-c2nc(-c3ccccc3)nc(-c3ccc4c(ccc5sc6ccccc6c54)c3)n2)cc1 ; c1ccc(-c2nc(-c3ccccc3)nc(-c3cccc4c3ccc3sc5ccccc5c34)n2)cc1 ; c1ccc(-c2nc(-c3ccc4cc5c(cc4c3)oc3ccccc35)nc(-c3cccc4c3ccc3ccccc34)n2)cc1 ; c1ccc(-c2nc(-c3ccc4cc5oc6ccccc6c5cc4c3)nc(-c3cc4ccccc4c4ccccc34)n2)cc1]

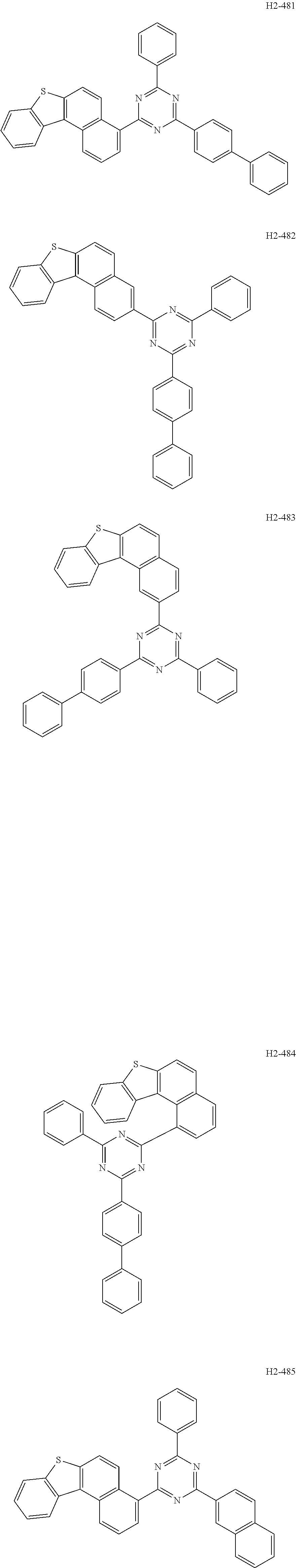 [SMILES: c1ccc(-c2ccc(-c3nc(-c4ccccc4)nc(-c4cccc5c4ccc4sc6ccccc6c45)n3)cc2)cc1 ; c1ccc(-c2ccc(-c3nc(-c4ccccc4)nc(-c4ccc5c(ccc6sc7ccccc7c65)c4)n3)cc2)cc1 ; c1ccc(-c2ccc(-c3nc(-c4ccccc4)nc(-c4ccc5ccc6sc7ccccc7c6c5c4)n3)cc2)cc1 ; c1ccc(-c2ccc(-c3nc(-c4ccccc4)nc(-c4cccc5ccc6sc7ccccc7c6c45)n3)cc2)cc1 ; c1ccc(-c2nc(-c3ccc4ccccc4c3)nc(-c3cccc4c3ccc3sc5ccccc5c34)n2)cc1]

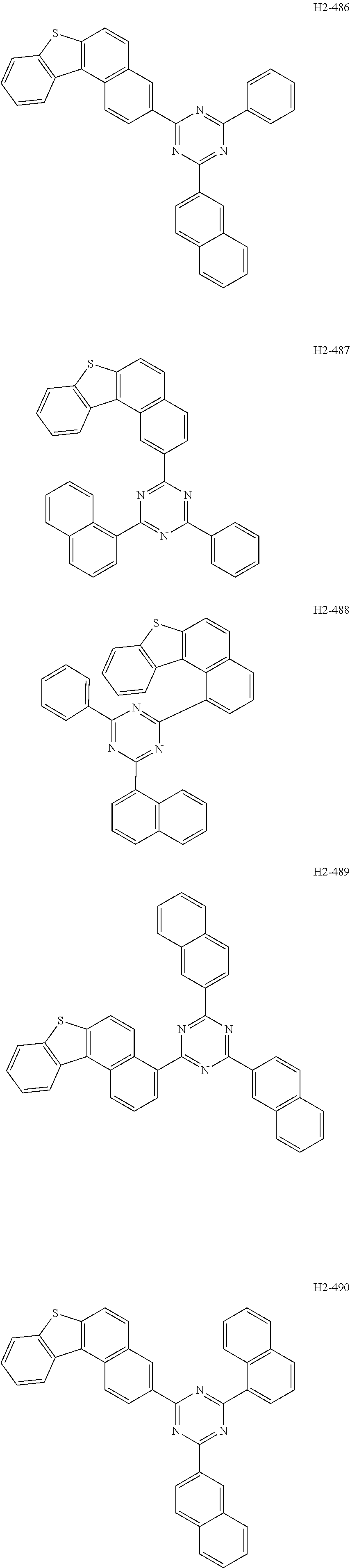 [SMILES: c1ccc(-c2nc(-c3ccc4ccccc4c3)nc(-c3ccc4c(ccc5sc6ccccc6c54)c3)n2)cc1 ; c1ccc(-c2nc(-c3ccc4ccc5sc6ccccc6c5c4c3)nc(-c3cccc4ccccc34)n2)cc1 ; c1ccc(-c2nc(-c3cccc4ccccc34)nc(-c3cccc4ccc5sc6ccccc6c5c34)n2)cc1 ; c1ccc2cc(-c3nc(-c4ccc5ccccc5c4)nc(-c4cccc5c4ccc4sc6ccccc6c45)n3)ccc2c1 ; c1ccc2cc(-c3nc(-c4ccc5c(ccc6sc7ccccc7c65)c4)nc(-c4cccc5ccccc45)n3)ccc2c1]

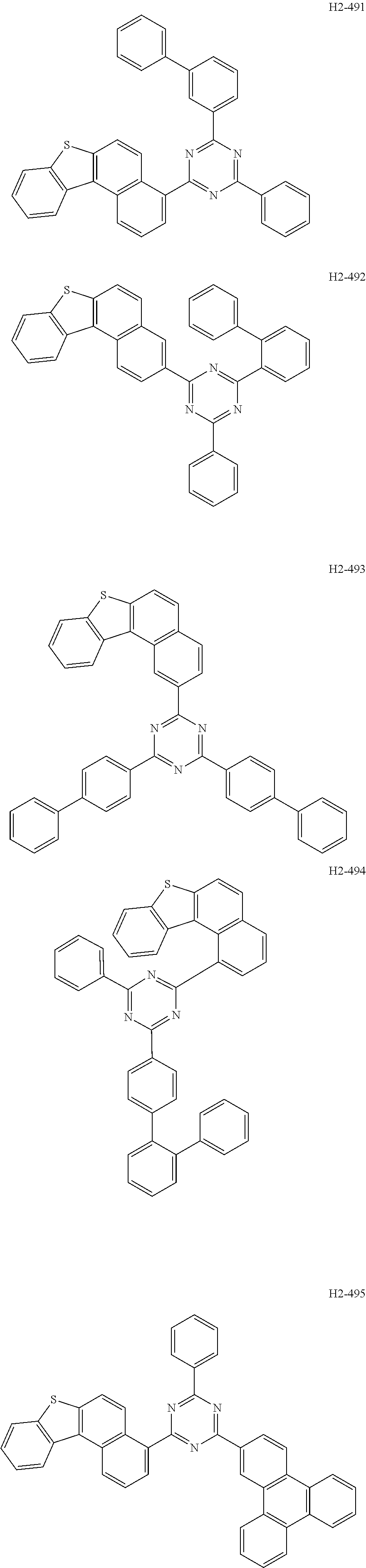 [SMILES: c1ccc(-c2cccc(-c3nc(-c4ccccc4)nc(-c4cccc5c4ccc4sc6ccccc6c45)n3)c2)cc1 ; c1ccc(-c2nc(-c3ccc4c(ccc5sc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6sc7ccccc7c6c5c4)n3)cc2)cc1 ; c1ccc(-c2nc(-c3ccc(-c4ccccc4-c4ccccc4)cc3)nc(-c3cccc4ccc5sc6ccccc6c5c34)n2)cc1 ; c1ccc(-c2nc(-c3ccc4c5ccccc5c5ccccc5c4c3)nc(-c3cccc4c3ccc3sc5ccccc5c34)n2)cc1]

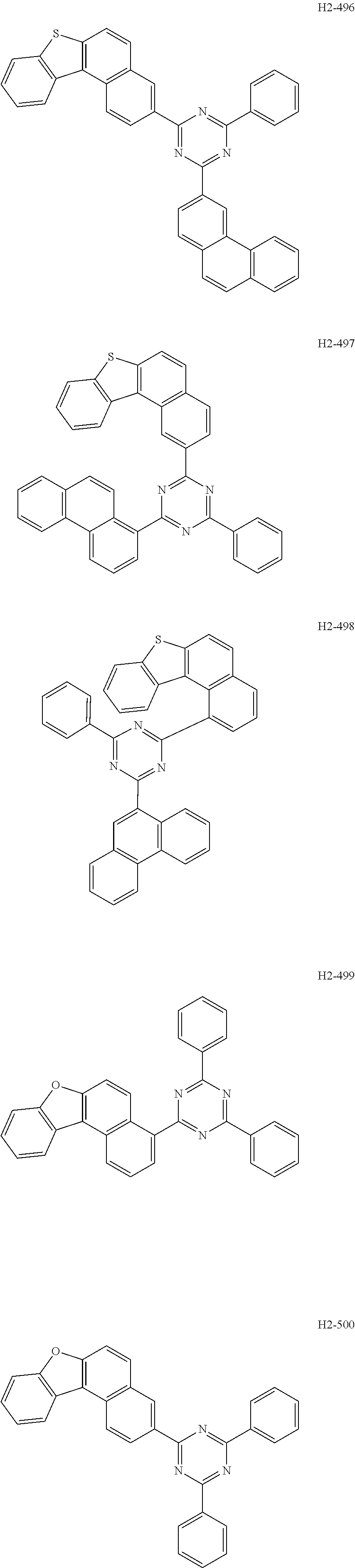 [SMILES: c1ccc(-c2nc(-c3ccc4c(ccc5sc6ccccc6c54)c3)nc(-c3ccc4ccc5ccccc5c4c3)n2)cc1 ; c1ccc(-c2nc(-c3ccc4ccc5sc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5sc6ccccc6c5c34)n2)cc1 ; c1ccc(-c2nc(-c3ccccc3)nc(-c3cccc4c3ccc3oc5ccccc5c34)n2)cc1 ; c1ccc(-c2nc(-c3ccccc3)nc(-c3ccc4c(ccc5oc6ccccc6c54)c3)n2)cc1]

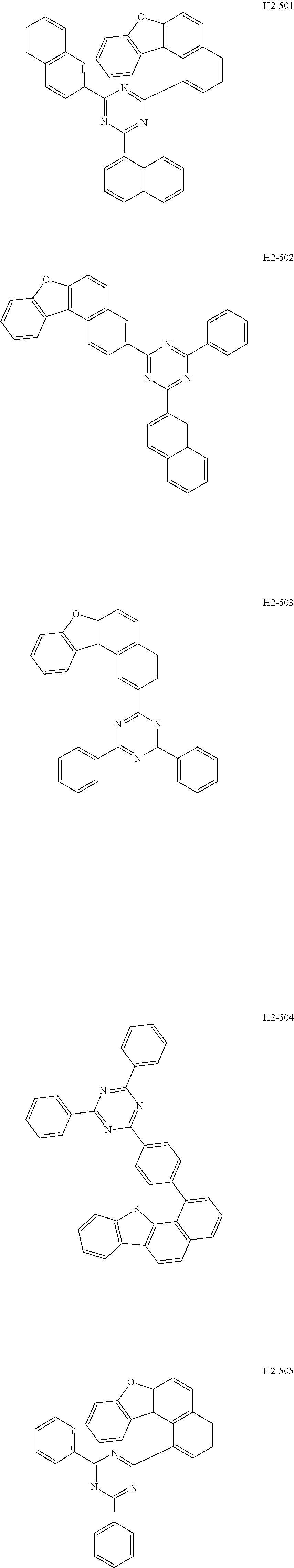 [SMILES: c1ccc2cc(-c3nc(-c4cccc5ccccc45)nc(-c4cccc5ccc6oc7ccccc7c6c45)n3)ccc2c1 ; c1ccc(-c2nc(-c3ccc4ccccc4c3)nc(-c3ccc4c(ccc5oc6ccccc6c54)c3)n2)cc1 ; c1ccc(-c2nc(-c3ccccc3)nc(-c3ccc4ccc5oc6ccccc6c5c4c3)n2)cc1 ; c1ccc(-c2nc(-c3ccccc3)nc(-c3ccc(-c4cccc5ccc6c7ccccc7sc6c45)cc3)n2)cc1 ; c1ccc(-c2nc(-c3ccccc3)nc(-c3cccc4ccc5oc6ccccc6c5c34)n2)cc1]

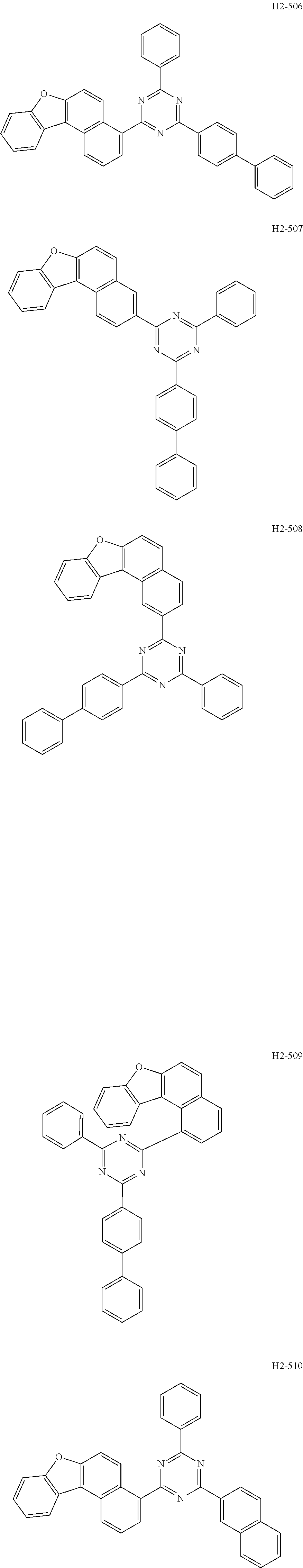 [SMILES: c1ccc(-c2ccc(-c3nc(-c4ccccc4)nc(-c4cccc5c4ccc4oc6ccccc6c45)n3)cc2)cc1 ; c1ccc(-c2ccc(-c3nc(-c4ccccc4)nc(-c4ccc5c(ccc6oc7ccccc7c65)c4)n3)cc2)cc1 ; c1ccc(-c2ccc(-c3nc(-c4ccccc4)nc(-c4ccc5ccc6oc7ccccc7c6c5c4)n3)cc2)cc1 ; c1ccc(-c2ccc(-c3nc(-c4ccccc4)nc(-c4cccc5ccc6oc7ccccc7c6c45)n3)cc2)cc1 ; c1ccc(-c2nc(-c3ccc4ccccc4c3)nc(-c3cccc4c3ccc3oc5ccccc5c34)n2)cc1]

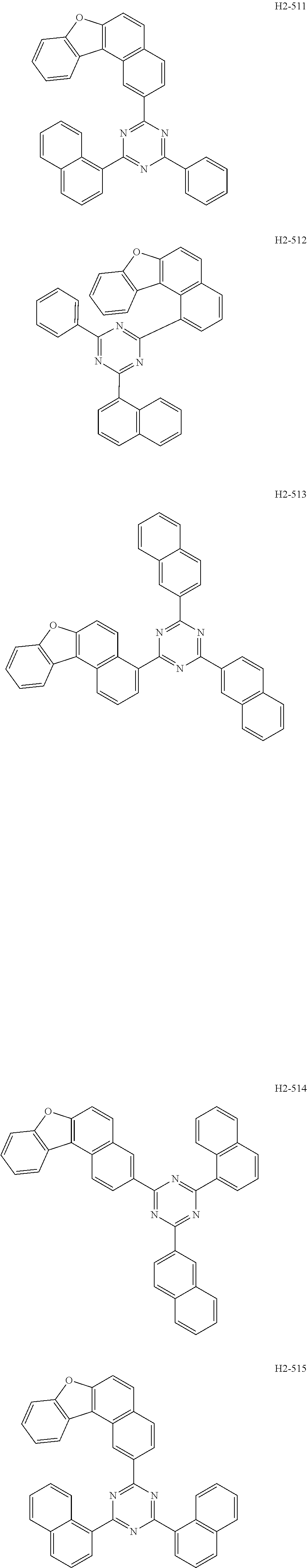 [SMILES: c1ccc(-c2nc(-c3ccc4ccc5oc6ccccc6c5c4c3)nc(-c3cccc4ccccc34)n2)cc1 ; c1ccc(-c2nc(-c3cccc4ccccc34)nc(-c3cccc4ccc5oc6ccccc6c5c34)n2)cc1 ; c1ccc2cc(-c3nc(-c4ccc5ccccc5c4)nc(-c4cccc5c4ccc4oc6ccccc6c45)n3)ccc2c1 ; c1ccc2cc(-c3nc(-c4ccc5c(ccc6oc7ccccc7c65)c4)nc(-c4cccc5ccccc45)n3)ccc2c1 ; c1ccc2c(-c3nc(-c4ccc5ccc6oc7ccccc7c6c5c4)nc(-c4cccc5ccccc45)n3)cccc2c1]

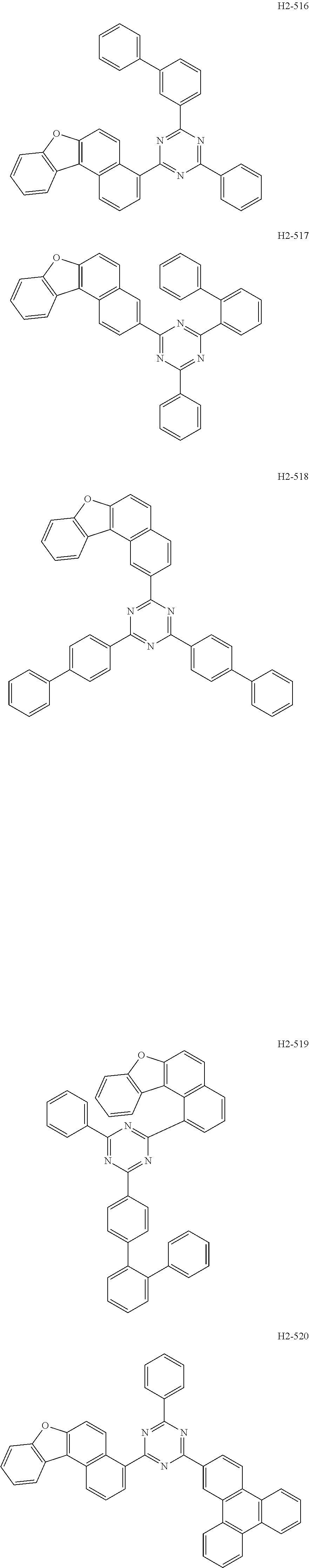 [SMILES: c1ccc(-c2cccc(-c3nc(-c4ccccc4)nc(-c4cccc5c4ccc4oc6ccccc6c45)n3)c2)cc1 ; c1ccc(-c2nc(-c3ccc4c(ccc5oc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6oc7ccccc7c6c5c4)n3)cc2)cc1 ; c1ccc(-c2nc(-c3ccc(-c4ccccc4-c4ccccc4)cc3)nc(-c3cccc4ccc5oc6ccccc6c5c34)n2)cc1 ; c1ccc(-c2nc(-c3ccc4c5ccccc5c5ccccc5c4c3)nc(-c3cccc4c3ccc3oc5ccccc5c34)n2)cc1]

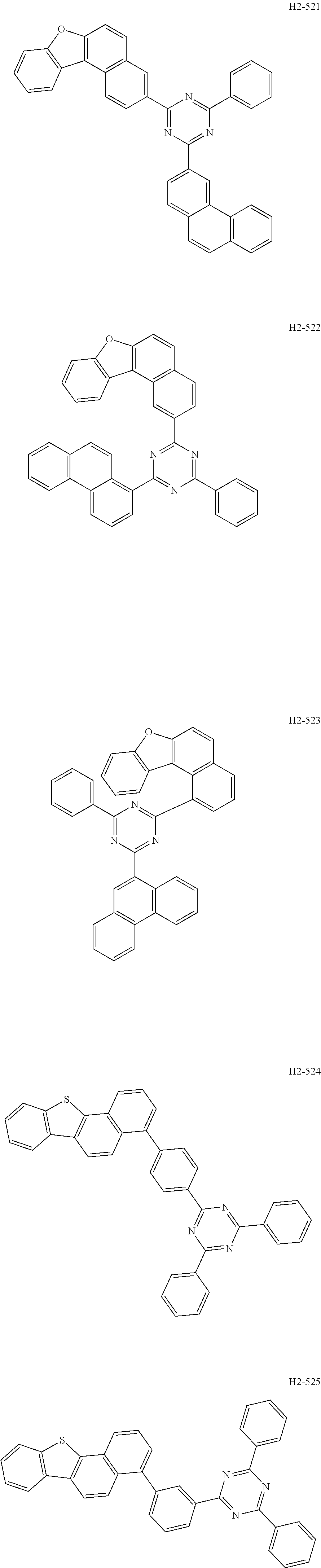 [SMILES: c1ccc(-c2nc(-c3ccc4c(ccc5oc6ccccc6c54)c3)nc(-c3ccc4ccc5ccccc5c4c3)n2)cc1 ; c1ccc(-c2nc(-c3ccc4ccc5oc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5oc6ccccc6c5c34)n2)cc1 ; c1ccc(-c2nc(-c3ccccc3)nc(-c3ccc(-c4cccc5c4ccc4c6ccccc6sc54)cc3)n2)cc1 ; c1ccc(-c2nc(-c3ccccc3)nc(-c3cccc(-c4cccc5c4ccc4c6ccccc6sc54)c3)n2)cc1]

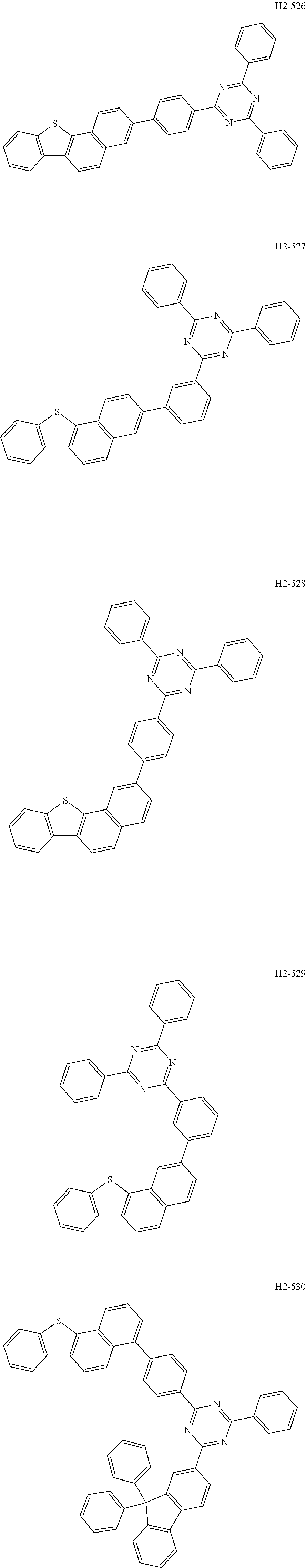 [SMILES: c1ccc(-c2nc(-c3ccccc3)nc(-c3ccc(-c4ccc5c(ccc6c7ccccc7sc56)c4)cc3)n2)cc1 ; c1ccc(-c2nc(-c3ccccc3)nc(-c3cccc(-c4ccc5c(ccc6c7ccccc7sc56)c4)c3)n2)cc1 ; c1ccc(-c2nc(-c3ccccc3)nc(-c3ccc(-c4ccc5ccc6c7ccccc7sc6c5c4)cc3)n2)cc1 ; c1ccc(-c2nc(-c3ccccc3)nc(-c3cccc(-c4ccc5ccc6c7ccccc7sc6c5c4)c3)n2)cc1 ; c1ccc(-c2nc(-c3ccc(-c4cccc5c4ccc4c6ccccc6sc54)cc3)nc(-c3ccc4c(c3)C(c3ccccc3)(c3ccccc3)c3ccccc3-4)n2)cc1]

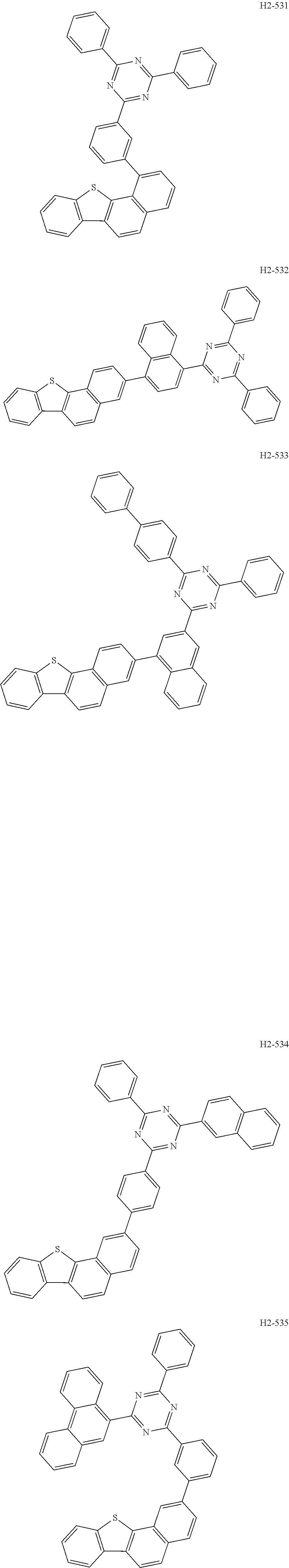 [SMILES: c1ccc(-c2nc(-c3ccccc3)nc(-c3cccc(-c4cccc5ccc6c7ccccc7sc6c45)c3)n2)cc1 ; c1ccc(-c2nc(-c3ccccc3)nc(-c3ccc(-c4ccc5c(ccc6c7ccccc7sc56)c4)c4ccccc34)n2)cc1 ; c1ccc(-c2ccc(-c3nc(-c4ccccc4)nc(-c4cc(-c5ccc6c(ccc7c8ccccc8sc67)c5)c5ccccc5c4)n3)cc2)cc1 ; c1ccc(-c2nc(-c3ccc(-c4ccc5ccc6c7ccccc7sc6c5c4)cc3)nc(-c3ccc4ccccc4c3)n2)cc1 ; c1ccc(-c2nc(-c3cccc(-c4ccc5ccc6c7ccccc7sc6c5c4)c3)nc(-c3cc4ccccc4c4ccccc34)n2)cc1]

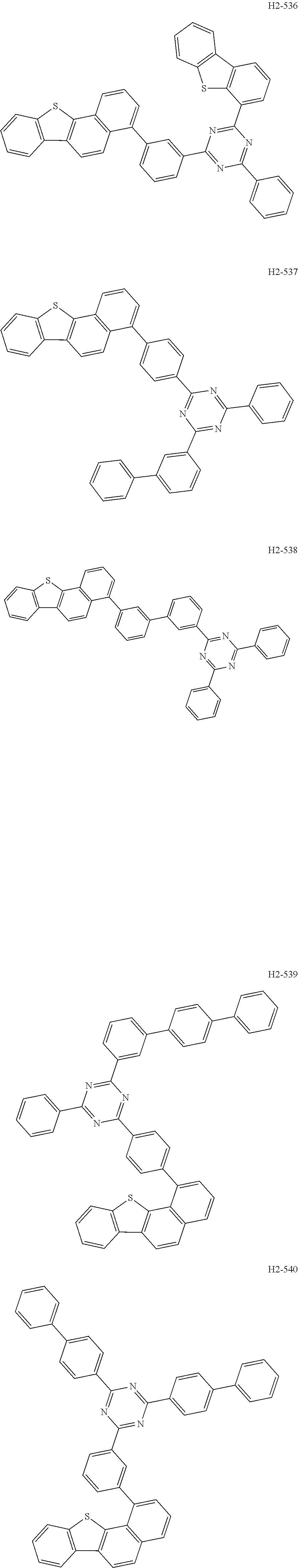 [SMILES: c1ccc(-c2nc(-c3cccc(-c4cccc5c4ccc4c6ccccc6sc54)c3)nc(-c3cccc4c3sc3ccccc34)n2)cc1 ; c1ccc(-c2cccc(-c3nc(-c4ccccc4)nc(-c4ccc(-c5cccc6c5ccc5c7ccccc7sc65)cc4)n3)c2)cc1 ; c1ccc(-c2nc(-c3ccccc3)nc(-c3cccc(-c4cccc(-c5cccc6c5ccc5c7ccccc7sc65)c4)c3)n2)cc1 ; c1ccc(-c2ccc(-c3cccc(-c4nc(-c5ccccc5)nc(-c5ccc(-c6cccc7ccc8c9ccccc9sc8c67)cc5)n4)c3)cc2)cc1 ; c1ccc(-c2ccc(-c3nc(-c4ccc(-c5ccccc5)cc4)nc(-c4cccc(-c5cccc6ccc7c8ccccc8sc7c56)c4)n3)cc2)cc1]

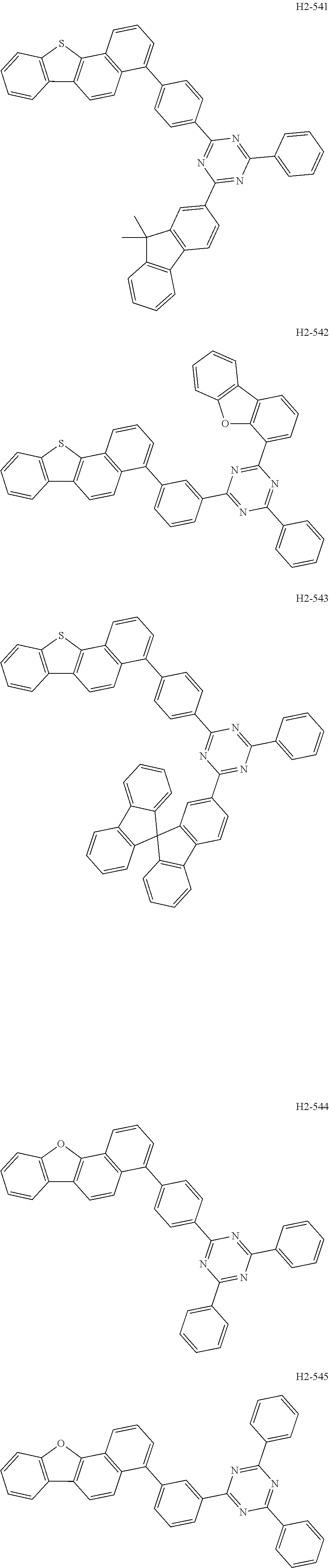 [SMILES: CC1(C)c2ccccc2-c2ccc(-c3nc(-c4ccccc4)nc(-c4ccc(-c5cccc6c5ccc5c7ccccc7sc65)cc4)n3)cc21 ; c1ccc(-c2nc(-c3cccc(-c4cccc5c4ccc4c6ccccc6sc54)c3)nc(-c3cccc4c3oc3ccccc34)n2)cc1 ; c1ccc(-c2nc(-c3ccc(-c4cccc5c4ccc4c6ccccc6sc54)cc3)nc(-c3ccc4c(c3)C3(c5ccccc5-c5ccccc53)c3ccccc3-4)n2)cc1 ; c1ccc(-c2nc(-c3ccccc3)nc(-c3ccc(-c4cccc5c4ccc4c6ccccc6oc54)cc3)n2)cc1 ; c1ccc(-c2nc(-c3ccccc3)nc(-c3cccc(-c4cccc5c4ccc4c6ccccc6oc54)c3)n2)cc1]

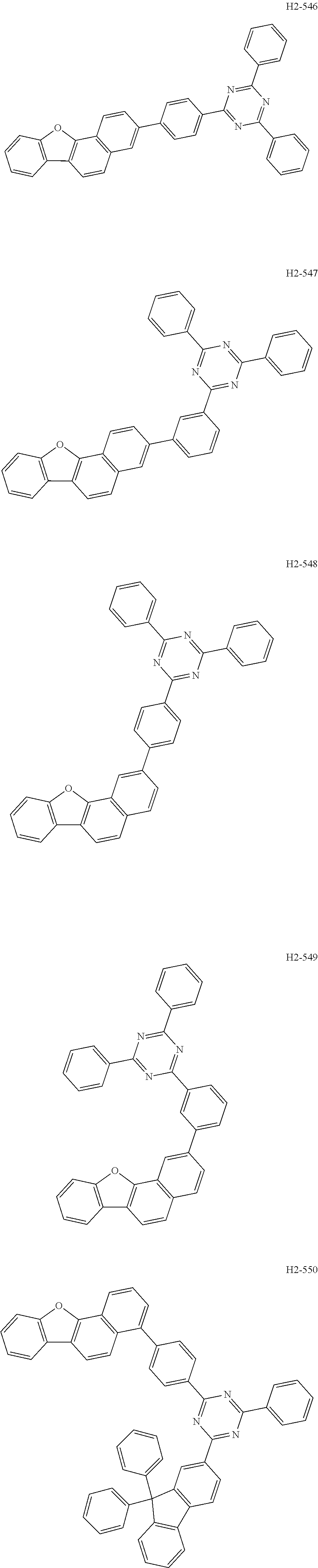 [SMILES: c1ccc(-c2nc(-c3ccccc3)nc(-c3ccc(-c4ccc5c(ccc6c7ccccc7oc56)c4)cc3)n2)cc1 ; c1ccc(-c2nc(-c3ccccc3)nc(-c3cccc(-c4ccc5c(ccc6c7ccccc7oc56)c4)c3)n2)cc1 ; c1ccc(-c2nc(-c3ccccc3)nc(-c3ccc(-c4ccc5ccc6c7ccccc7oc6c5c4)cc3)n2)cc1 ; c1ccc(-c2nc(-c3ccccc3)nc(-c3cccc(-c4ccc5ccc6c7ccccc7oc6c5c4)c3)n2)cc1 ; c1ccc(-c2nc(-c3ccc(-c4cccc5c4ccc4c6ccccc6oc54)cc3)nc(-c3ccc4c(c3)C(c3ccccc3)(c3ccccc3)c3ccccc3-4)n2)cc1]

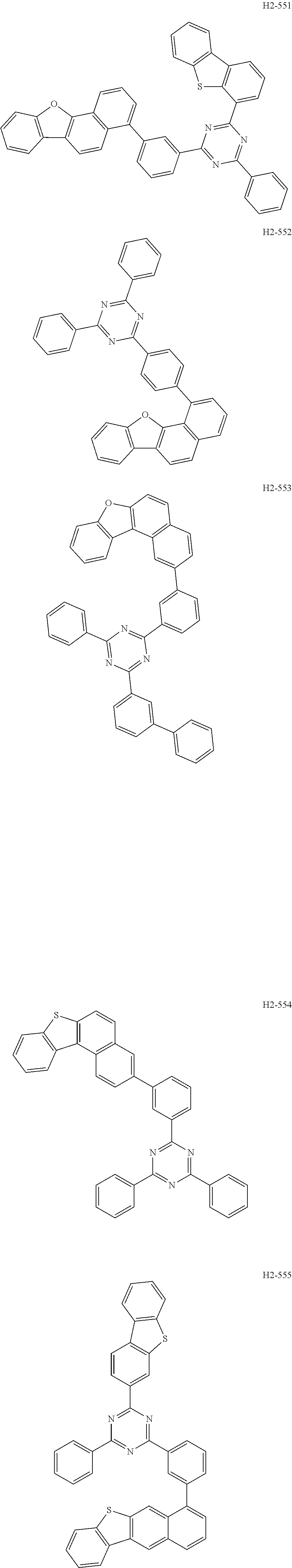 [SMILES: c1ccc(-c2nc(-c3cccc(-c4cccc5c4ccc4c6ccccc6oc54)c3)nc(-c3cccc4c3sc3ccccc34)n2)cc1 ; c1ccc(-c2nc(-c3ccccc3)nc(-c3ccc(-c4cccc5ccc6c7ccccc7oc6c45)cc3)n2)cc1 ; c1ccc(-c2cccc(-c3nc(-c4ccccc4)nc(-c4cccc(-c5ccc6ccc7oc8ccccc8c7c6c5)c4)n3)c2)cc1 ; c1ccc(-c2nc(-c3ccccc3)nc(-c3cccc(-c4ccc5c(ccc6sc7ccccc7c65)c4)c3)n2)cc1 ; c1ccc(-c2nc(-c3cccc(-c4cccc5cc6c(cc45)sc4ccccc46)c3)nc(-c3ccc4c(c3)sc3ccccc34)n2)cc1]

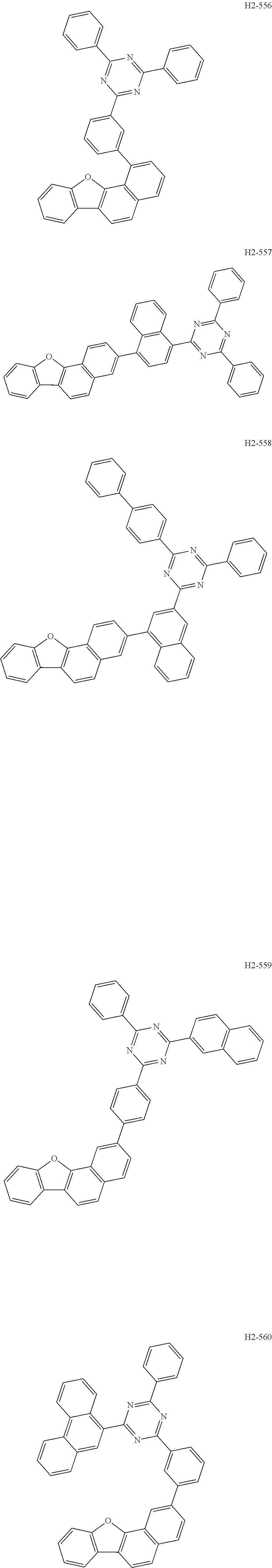 [SMILES: c1ccc(-c2nc(-c3ccccc3)nc(-c3cccc(-c4cccc5ccc6c7ccccc7oc6c45)c3)n2)cc1 ; c1ccc(-c2nc(-c3ccccc3)nc(-c3ccc(-c4ccc5c(ccc6c7ccccc7oc56)c4)c4ccccc34)n2)cc1 ; c1ccc(-c2ccc(-c3nc(-c4ccccc4)nc(-c4cc(-c5ccc6c(ccc7c8ccccc8oc67)c5)c5ccccc5c4)n3)cc2)cc1 ; c1ccc(-c2nc(-c3ccc(-c4ccc5ccc6c7ccccc7oc6c5c4)cc3)nc(-c3ccc4ccccc4c3)n2)cc1 ; c1ccc(-c2nc(-c3cccc(-c4ccc5ccc6c7ccccc7oc6c5c4)c3)nc(-c3cc4ccccc4c4ccccc34)n2)cc1]

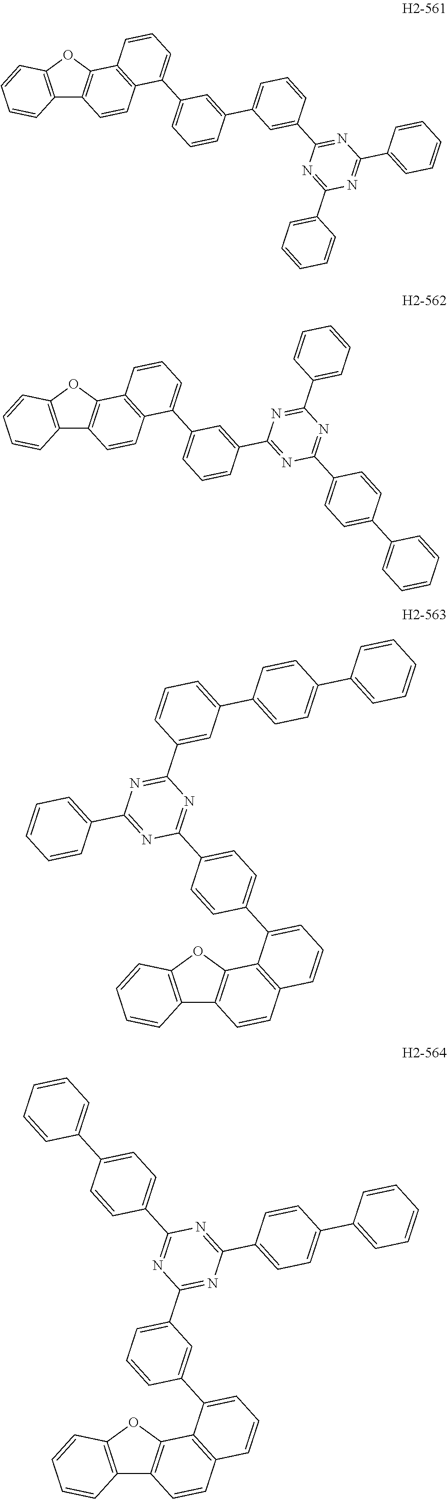 [SMILES: c1ccc(-c2nc(-c3ccccc3)nc(-c3cccc(-c4cccc(-c5cccc6c5ccc5c7ccccc7oc65)c4)c3)n2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2ccc(-c3cccc(-c4nc(-c5ccccc5)nc(-c5ccc(-c6cccc7ccc8c9ccccc9oc8c67)cc5)n4)c3)cc2)cc1 ; c1ccc(-c2ccc(-c3nc(-c4ccc(-c5ccccc5)cc4)nc(-c4cccc(-c5cccc6ccc7c8ccccc8oc7c56)c4)n3)cc2)cc1]

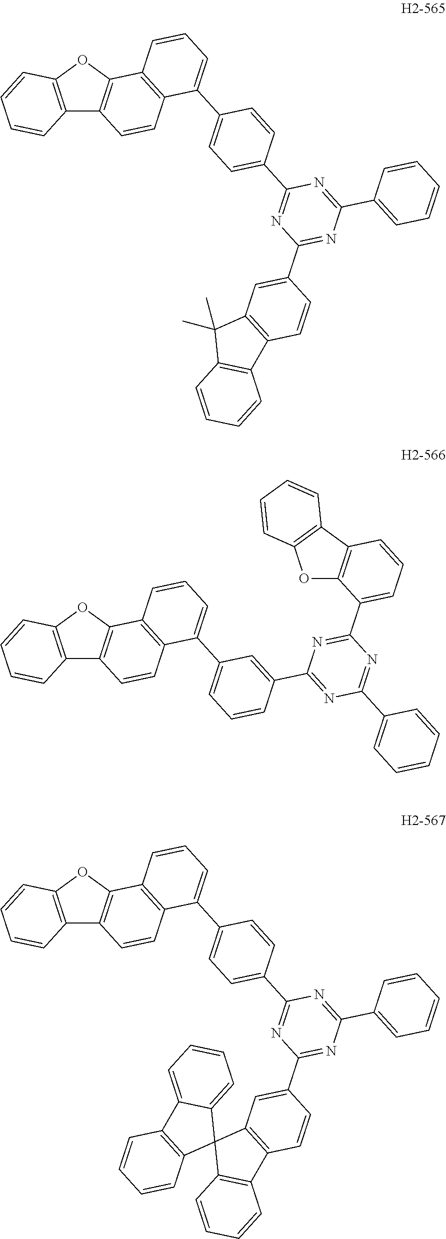 [SMILES: CC1(C)c2ccccc2-c2ccc(-c3nc(-c4ccccc4)nc(-c4ccc(-c5cccc6c5ccc5c7ccccc7oc65)cc4)n3)cc21 ; c1ccc(-c2nc(-c3cccc(-c4cccc5c4ccc4c6ccccc6oc54)c3)nc(-c3cccc4c3oc3ccccc34)n2)cc1 ; c1ccc(-c2nc(-c3ccc(-c4cccc5c4ccc4c6ccccc6oc54)cc3)nc(-c3ccc4c(c3)C3(c5ccccc5-c5ccccc53)c3ccccc3-4)n2)cc1]

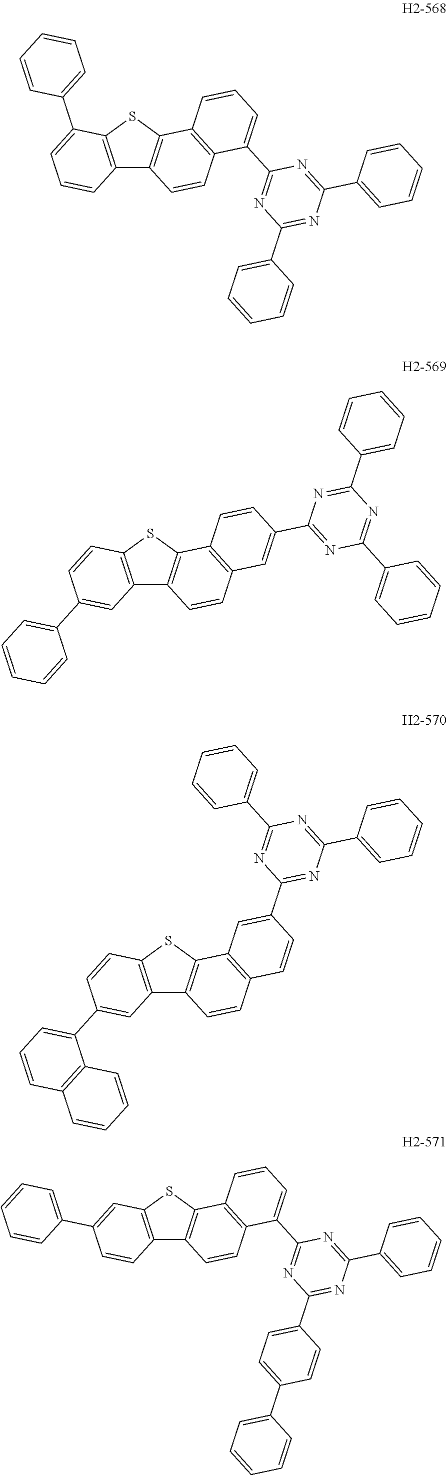 [SMILES: c1ccc(-c2nc(-c3ccccc3)nc(-c3cccc4c3ccc3c5cccc(-c6ccccc6)c5sc43)n2)cc1 ; c1ccc(-c2ccc3sc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6sc5c4c3)n2)cc1 ; c1ccc(-c2ccc(-c3nc(-c4ccccc4)nc(-c4cccc5c4ccc4c6ccc(-c7ccccc7)cc6sc54)n3)cc2)cc1]

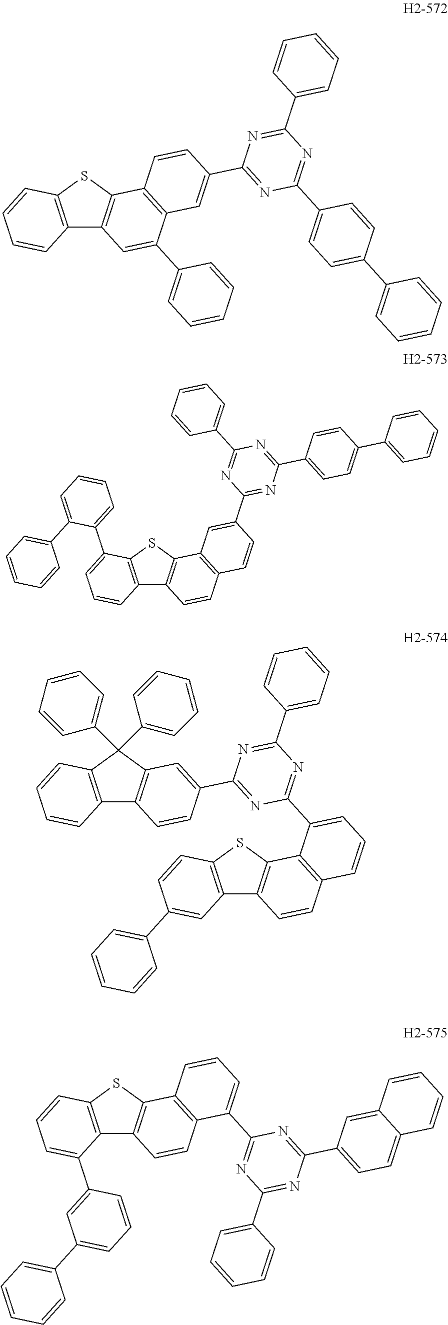 [SMILES: c1ccc(-c2ccc(-c3nc(-c4ccccc4)nc(-c4ccc5c(c4)c(-c4ccccc4)cc4c6ccccc6sc54)n3)cc2)cc1 ; c1ccc(-c2ccc(-c3nc(-c4ccccc4)nc(-c4ccc5ccc6c7cccc(-c8ccccc8-c8ccccc8)c7sc6c5c4)n3)cc2)cc1 ; c1ccc(-c2ccc3sc4c(ccc5cccc(-c6nc(-c7ccccc7)nc(-c7ccc8c(c7)C(c7ccccc7)(c7ccccc7)c7ccccc7-8)n6)c54)c3c2)cc1 ; c1ccc(-c2cccc(-c3cccc4sc5c6cccc(-c7nc(-c8ccccc8)nc(-c8ccc9ccccc9c8)n7)c6ccc5c34)c2)cc1]

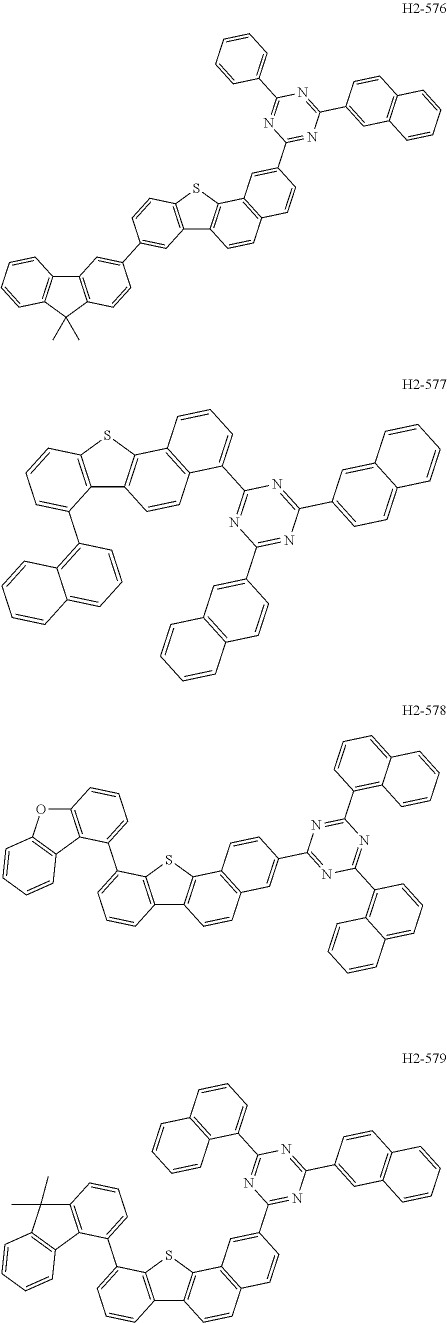 [SMILES: CC1(C)c2ccccc2-c2cc(-c3ccc4sc5c6cc(-c7nc(-c8ccccc8)nc(-c8ccc9ccccc9c8)n7)ccc6ccc5c4c3)ccc21 ; c1ccc2cc(-c3nc(-c4ccc5ccccc5c4)nc(-c4cccc5c4ccc4c5sc5cccc(-c6cccc7ccccc67)c54)n3)ccc2c1 ; c1ccc2c(-c3nc(-c4ccc5c(ccc6c7cccc(-c8cccc9oc%10ccccc%10c89)c7sc56)c4)nc(-c4cccc5ccccc45)n3)cccc2c1 ; CC1(C)c2ccccc2-c2c(-c3cccc4c3sc3c5cc(-c6nc(-c7ccc8ccccc8c7)nc(-c7cccc8ccccc78)n6)ccc5ccc43)cccc21]

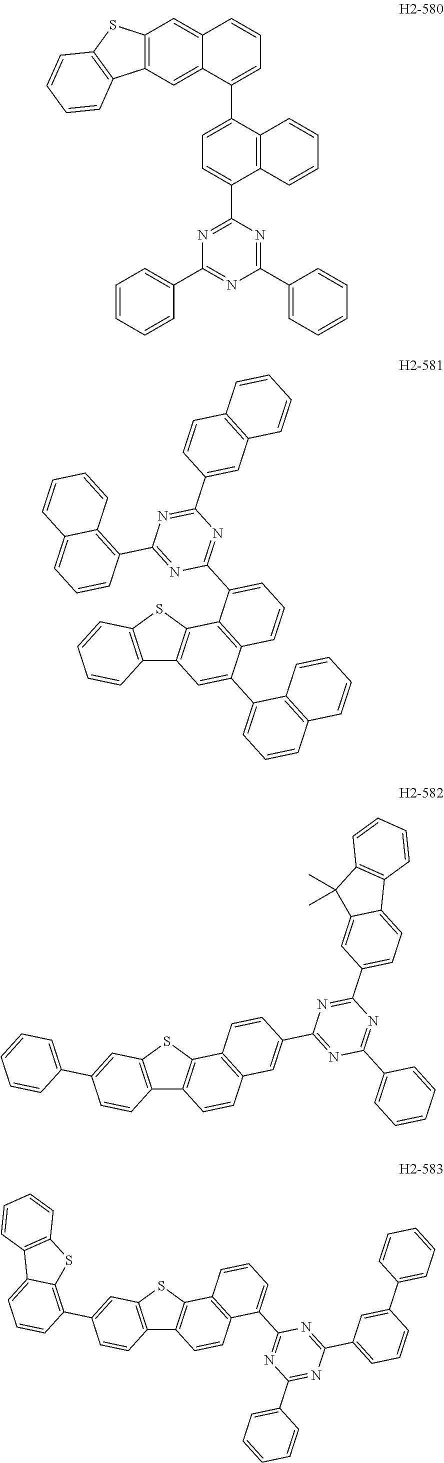 [SMILES: c1ccc(-c2nc(-c3ccccc3)nc(-c3ccc(-c4cccc5cc6sc7ccccc7c6cc45)c4ccccc34)n2)cc1 ; c1ccc2cc(-c3nc(-c4cccc5ccccc45)nc(-c4cccc5c(-c6cccc7ccccc67)cc6c7ccccc7sc6c45)n3)ccc2c1 ; CC1(C)c2ccccc2-c2ccc(-c3nc(-c4ccccc4)nc(-c4ccc5c(ccc6c7ccc(-c8ccccc8)cc7sc56)c4)n3)cc21 ; c1ccc(-c2cccc(-c3nc(-c4ccccc4)nc(-c4cccc5c4ccc4c6ccc(-c7cccc8c7sc7ccccc78)cc6sc54)n3)c2)cc1]

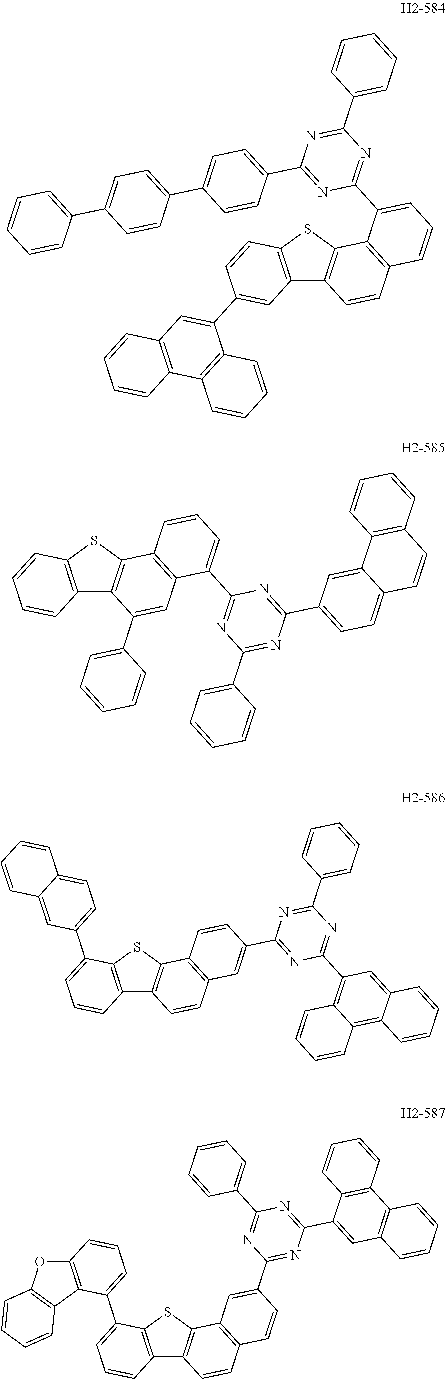 [SMILES: c1ccc(-c2ccc(-c3ccc(-c4nc(-c5ccccc5)nc(-c5cccc6ccc7c8cc(-c9cc%10ccccc%10c%10ccccc9%10)ccc8sc7c56)n4)cc3)cc2)cc1 ; c1ccc(-c2nc(-c3ccc4ccc5ccccc5c4c3)nc(-c3cccc4c3cc(-c3ccccc3)c3c5ccccc5sc43)n2)cc1 ; c1ccc(-c2nc(-c3ccc4c(ccc5c6cccc(-c7ccc8ccccc8c7)c6sc45)c3)nc(-c3cc4ccccc4c4ccccc34)n2)cc1 ; c1ccc(-c2nc(-c3ccc4ccc5c6cccc(-c7cccc8oc9ccccc9c78)c6sc5c4c3)nc(-c3cc4ccccc4c4ccccc34)n2)cc1]

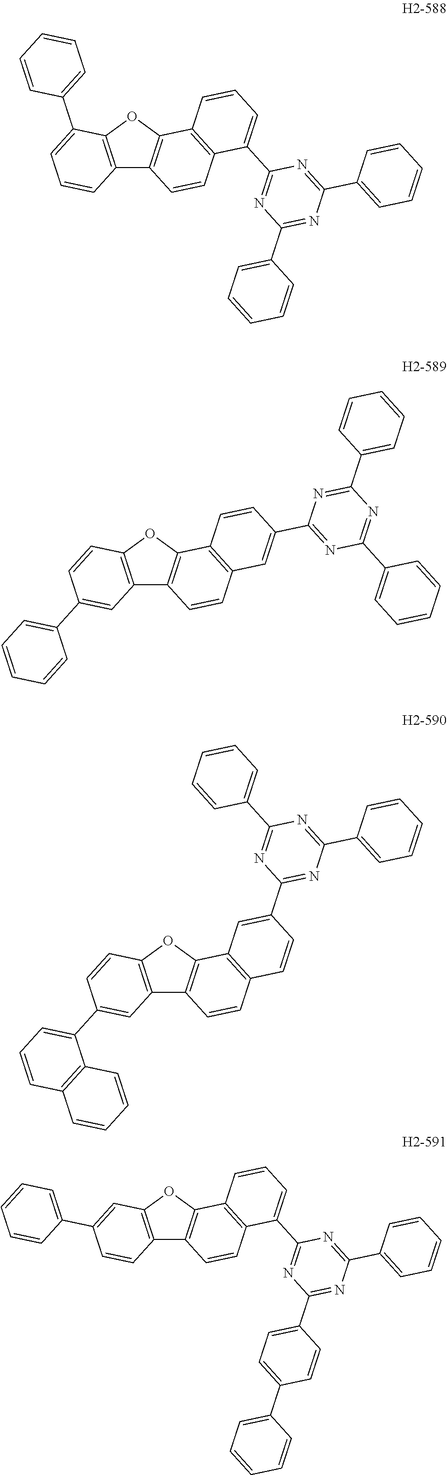 [SMILES: c1ccc(-c2nc(-c3ccccc3)nc(-c3cccc4c3ccc3c5cccc(-c6ccccc6)c5oc43)n2)cc1 ; c1ccc(-c2ccc3oc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6oc5c4c3)n2)cc1 ; c1ccc(-c2ccc(-c3nc(-c4ccccc4)nc(-c4cccc5c4ccc4c6ccc(-c7ccccc7)cc6oc54)n3)cc2)cc1]

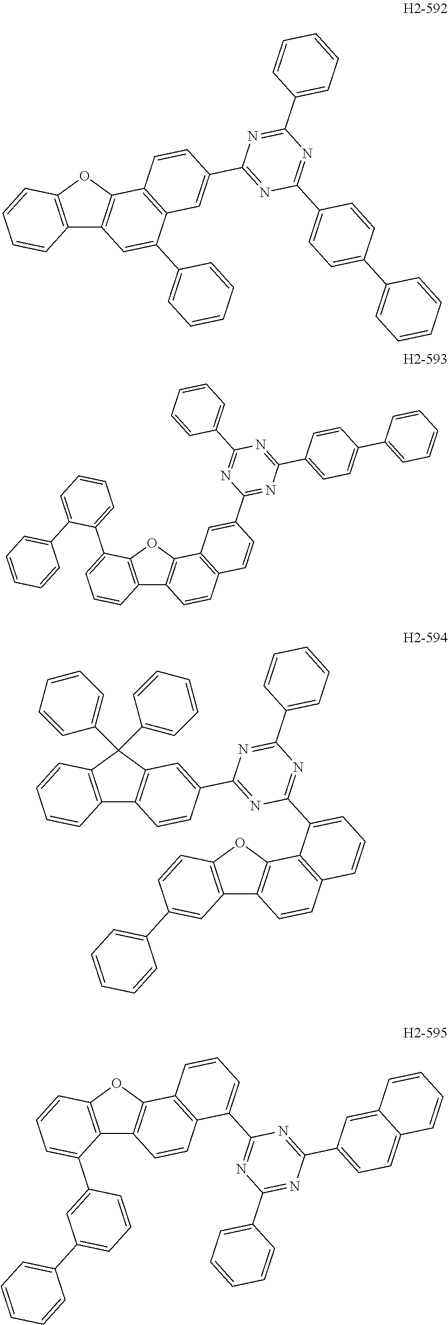 [SMILES: c1ccc(-c2ccc(-c3nc(-c4ccccc4)nc(-c4ccc5c(c4)c(-c4ccccc4)cc4c6ccccc6oc54)n3)cc2)cc1 ; c1ccc(-c2ccc(-c3nc(-c4ccccc4)nc(-c4ccc5ccc6c7cccc(-c8ccccc8-c8ccccc8)c7oc6c5c4)n3)cc2)cc1 ; c1ccc(-c2ccc3oc4c(ccc5cccc(-c6nc(-c7ccccc7)nc(-c7ccc8c(c7)C(c7ccccc7)(c7ccccc7)c7ccccc7-8)n6)c54)c3c2)cc1 ; c1ccc(-c2cccc(-c3cccc4oc5c6cccc(-c7nc(-c8ccccc8)nc(-c8ccc9ccccc9c8)n7)c6ccc5c34)c2)cc1]

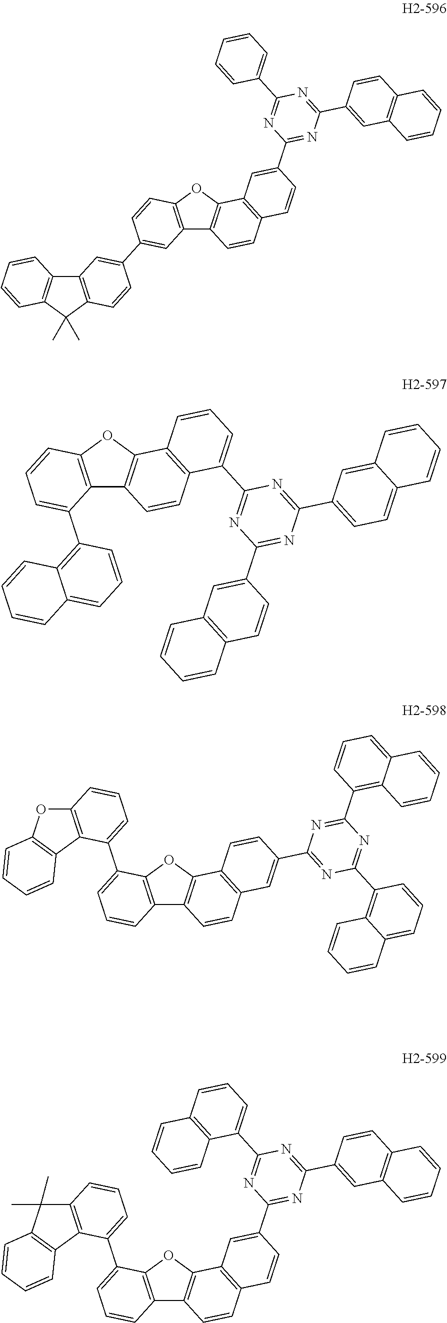 [SMILES: CC1(C)c2ccccc2-c2cc(-c3ccc4oc5c6cc(-c7nc(-c8ccccc8)nc(-c8ccc9ccccc9c8)n7)ccc6ccc5c4c3)ccc21 ; c1ccc2cc(-c3nc(-c4ccc5ccccc5c4)nc(-c4cccc5c4ccc4c5oc5cccc(-c6cccc7ccccc67)c54)n3)ccc2c1 ; c1ccc2c(-c3nc(-c4ccc5c(ccc6c7cccc(-c8cccc9oc%10ccccc%10c89)c7oc56)c4)nc(-c4cccc5ccccc45)n3)cccc2c1 ; CC1(C)c2ccccc2-c2c(-c3cccc4c3oc3c5cc(-c6nc(-c7ccc8ccccc8c7)nc(-c7cccc8ccccc78)n6)ccc5ccc43)cccc21]

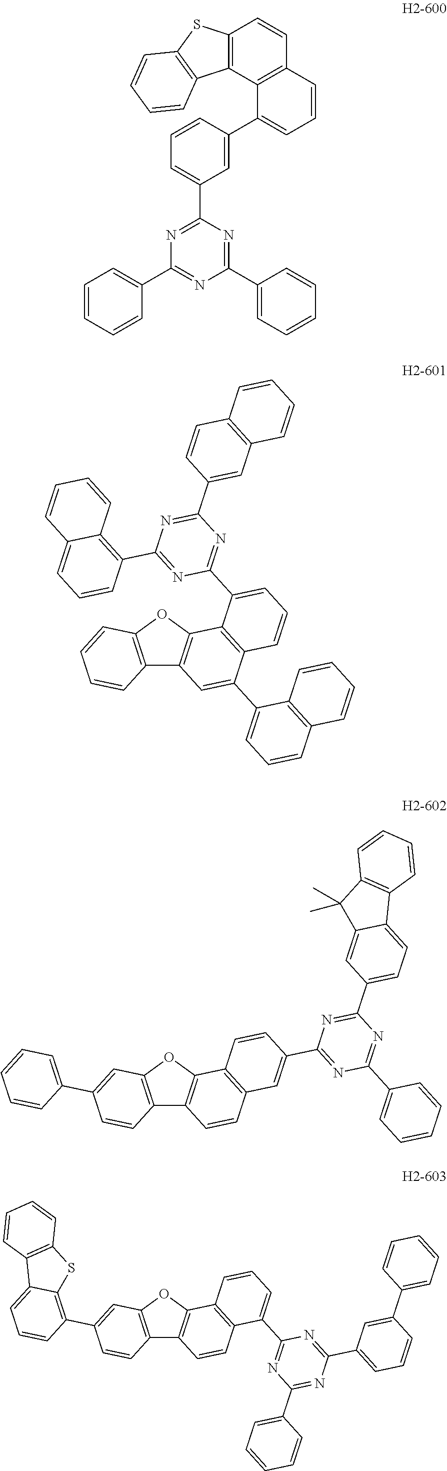 [SMILES: c1ccc(-c2nc(-c3ccccc3)nc(-c3cccc(-c4cccc5ccc6sc7ccccc7c6c45)c3)n2)cc1 ; c1ccc2cc(-c3nc(-c4cccc5ccccc45)nc(-c4cccc5c(-c6cccc7ccccc67)cc6c7ccccc7oc6c45)n3)ccc2c1 ; CC1(C)c2ccccc2-c2ccc(-c3nc(-c4ccccc4)nc(-c4ccc5c(ccc6c7ccc(-c8ccccc8)cc7oc56)c4)n3)cc21 ; c1ccc(-c2cccc(-c3nc(-c4ccccc4)nc(-c4cccc5c4ccc4c6ccc(-c7cccc8c7sc7ccccc78)cc6oc54)n3)c2)cc1]

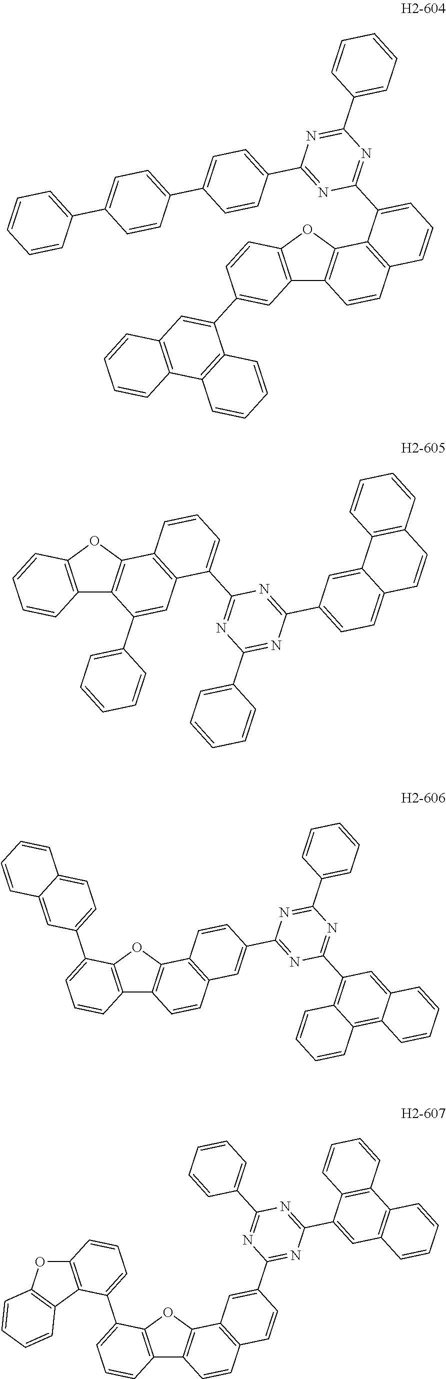 [SMILES: c1ccc(-c2ccc(-c3ccc(-c4nc(-c5ccccc5)nc(-c5cccc6ccc7c8cc(-c9cc%10ccccc%10c%10ccccc9%10)ccc8oc7c56)n4)cc3)cc2)cc1 ; c1ccc(-c2nc(-c3ccc4ccc5ccccc5c4c3)nc(-c3cccc4c3cc(-c3ccccc3)c3c5ccccc5oc43)n2)cc1 ; c1ccc(-c2nc(-c3ccc4c(ccc5c6cccc(-c7ccc8ccccc8c7)c6oc45)c3)nc(-c3cc4ccccc4c4ccccc34)n2)cc1 ; c1ccc(-c2nc(-c3ccc4ccc5c6cccc(-c7cccc8oc9ccccc9c78)c6oc5c4c3)nc(-c3cc4ccccc4c4ccccc34)n2)cc1]

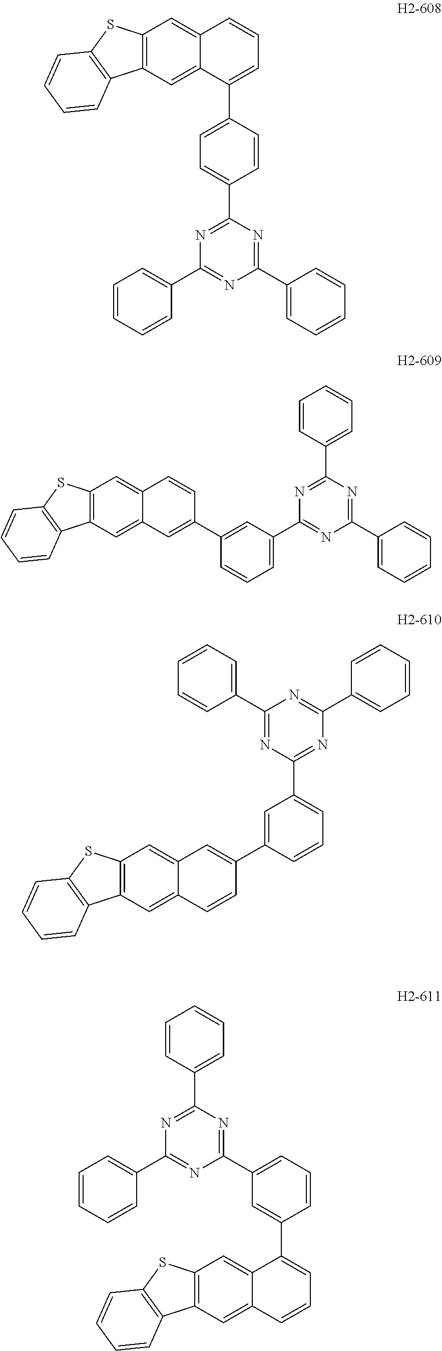 [SMILES: c1ccc(-c2nc(-c3ccccc3)nc(-c3ccc(-c4cccc5cc6sc7ccccc7c6cc45)cc3)n2)cc1 ; c1ccc(-c2nc(-c3ccccc3)nc(-c3cccc(-c4ccc5cc6sc7ccccc7c6cc5c4)c3)n2)cc1 ; c1ccc(-c2nc(-c3ccccc3)nc(-c3cccc(-c4ccc5cc6c(cc5c4)sc4ccccc46)c3)n2)cc1 ; c1ccc(-c2nc(-c3ccccc3)nc(-c3cccc(-c4cccc5cc6c(cc45)sc4ccccc46)c3)n2)cc1]

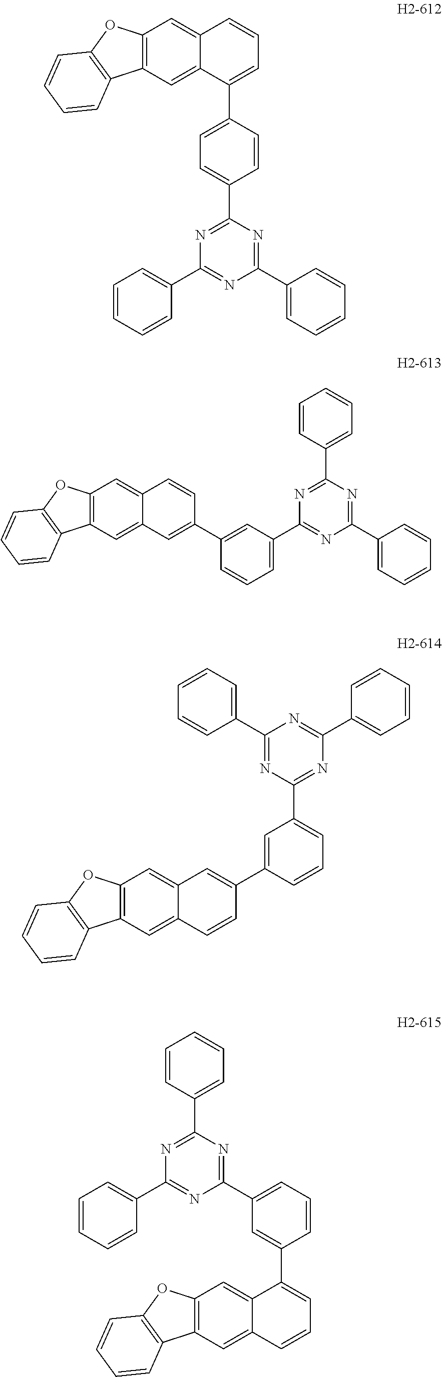 [SMILES: c1ccc(-c2nc(-c3ccccc3)nc(-c3ccc(-c4cccc5cc6oc7ccccc7c6cc45)cc3)n2)cc1 ; c1ccc(-c2nc(-c3ccccc3)nc(-c3cccc(-c4ccc5cc6oc7ccccc7c6cc5c4)c3)n2)cc1 ; c1ccc(-c2nc(-c3ccccc3)nc(-c3cccc(-c4ccc5cc6c(cc5c4)oc4ccccc46)c3)n2)cc1 ; c1ccc(-c2nc(-c3ccccc3)nc(-c3cccc(-c4cccc5cc6c(cc45)oc4ccccc46)c3)n2)cc1]

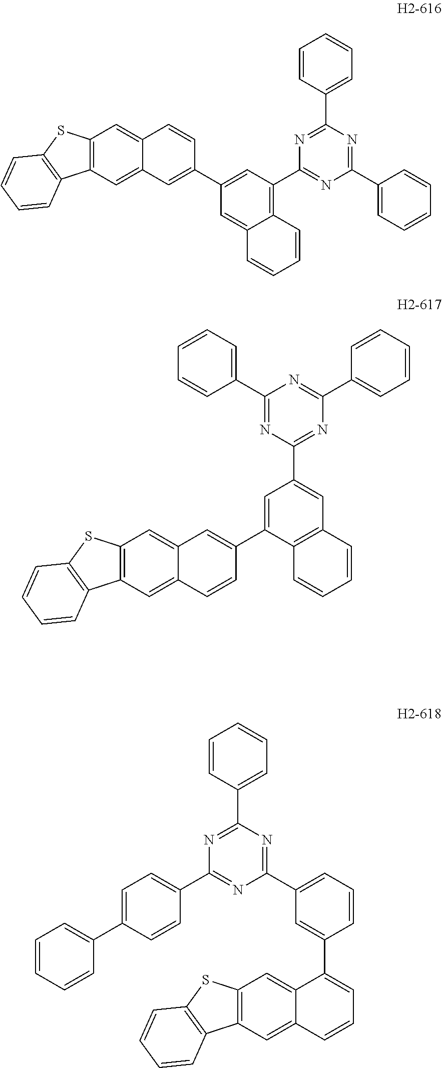 [SMILES: c1ccc(-c2nc(-c3ccccc3)nc(-c3cc(-c4ccc5cc6sc7ccccc7c6cc5c4)cc4ccccc34)n2)cc1 ; c1ccc(-c2nc(-c3ccccc3)nc(-c3cc(-c4ccc5cc6c(cc5c4)sc4ccccc46)c4ccccc4c3)n2)cc1 ; c1ccc(-c2ccc(-c3nc(-c4ccccc4)nc(-c4cccc(-c5cccc6cc7c(cc56)sc5ccccc57)c4)n3)cc2)cc1]

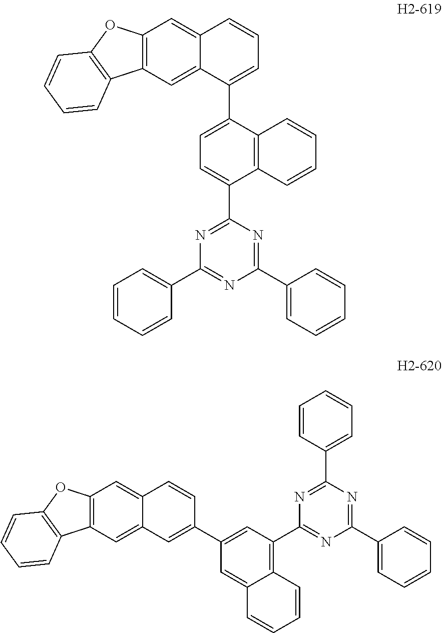 [SMILES: c1ccc(-c2nc(-c3ccccc3)nc(-c3ccc(-c4cccc5cc6oc7ccccc7c6cc45)c4ccccc34)n2)cc1 ; c1ccc(-c2nc(-c3ccccc3)nc(-c3cc(-c4ccc5cc6oc7ccccc7c6cc5c4)cc4ccccc34)n2)cc1]

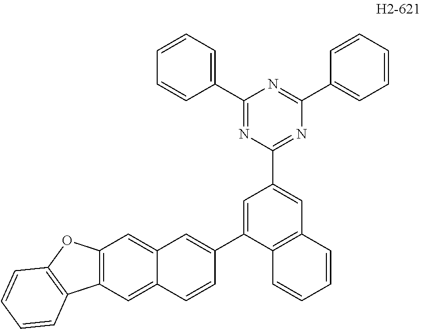 [SMILES: c1ccc(-c2nc(-c3ccccc3)nc(-c3cc(-c4ccc5cc6c(cc5c4)oc4ccccc46)c4ccccc4c3)n2)cc1]

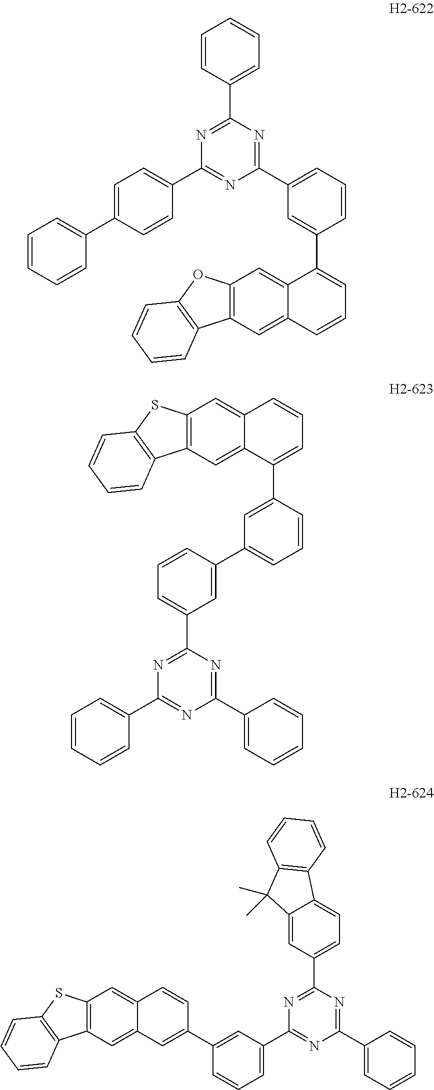

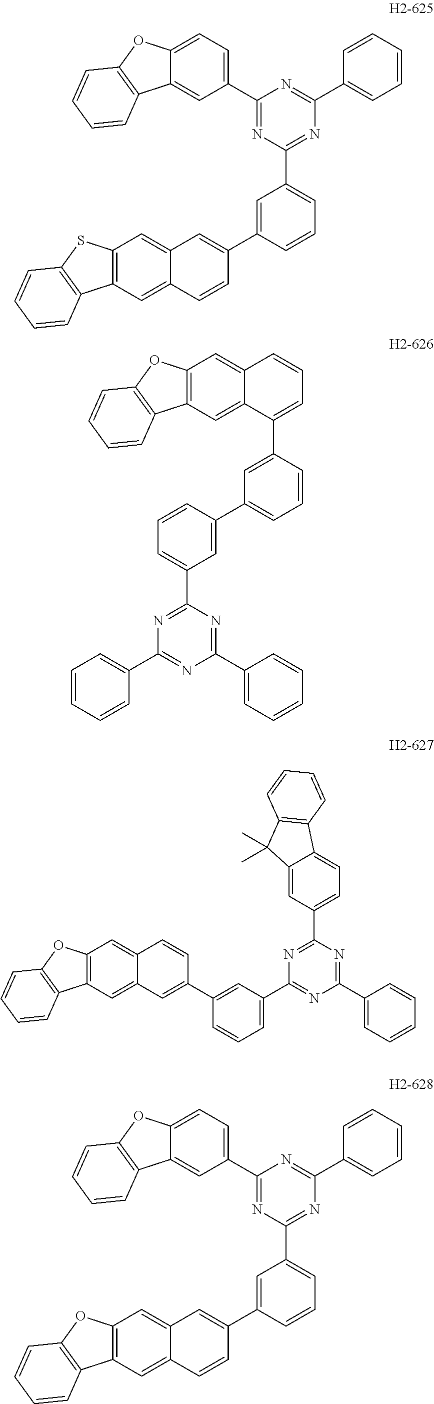 [SMILES: c1ccc(-c2nc(-c3cccc(-c4ccc5cc6c(cc5c4)sc4ccccc46)c3)nc(-c3ccc4oc5ccccc5c4c3)n2)cc1 ; c1ccc(-c2nc(-c3ccccc3)nc(-c3cccc(-c4cccc(-c5cccc6cc7oc8ccccc8c7cc56)c4)c3)n2)cc1 ; CC1(C)c2ccccc2-c2ccc(-c3nc(-c4ccccc4)nc(-c4cccc(-c5ccc6cc7oc8ccccc8c7cc6c5)c4)n3)cc21 ; c1ccc(-c2nc(-c3cccc(-c4ccc5cc6c(cc5c4)oc4ccccc46)c3)nc(-c3ccc4oc5ccccc5c4c3)n2)cc1]

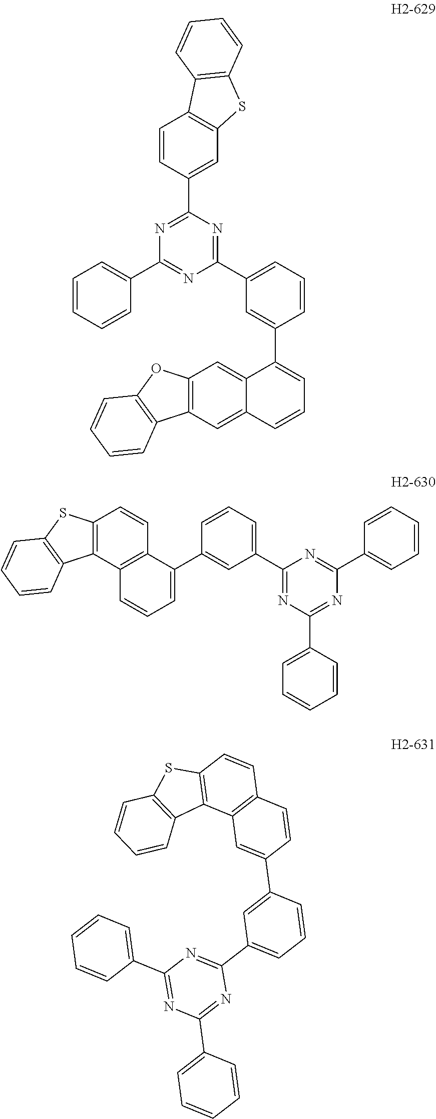 [SMILES: c1ccc(-c2nc(-c3cccc(-c4cccc5cc6c(cc45)oc4ccccc46)c3)nc(-c3ccc4c(c3)sc3ccccc34)n2)cc1 ; c1ccc(-c2nc(-c3ccccc3)nc(-c3cccc(-c4cccc5c4ccc4sc6ccccc6c45)c3)n2)cc1 ; c1ccc(-c2nc(-c3ccccc3)nc(-c3cccc(-c4ccc5ccc6sc7ccccc7c6c5c4)c3)n2)cc1]

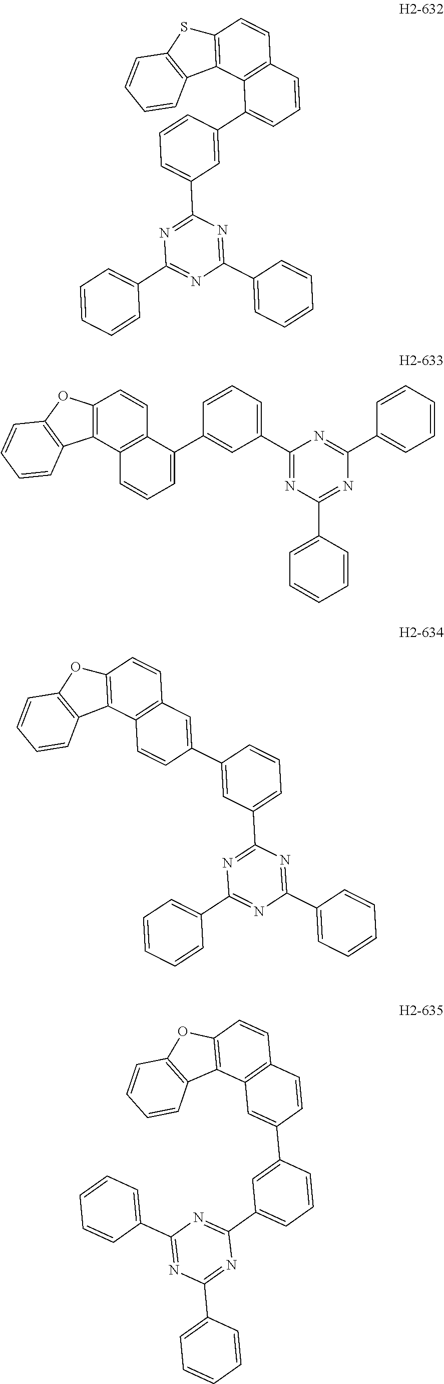 [SMILES: c1ccc(-c2nc(-c3ccccc3)nc(-c3cccc(-c4cccc5ccc6sc7ccccc7c6c45)c3)n2)cc1 ; c1ccc(-c2nc(-c3ccccc3)nc(-c3cccc(-c4cccc5c4ccc4oc6ccccc6c45)c3)n2)cc1 ; c1ccc(-c2nc(-c3ccccc3)nc(-c3cccc(-c4ccc5c(ccc6oc7ccccc7c65)c4)c3)n2)cc1 ; c1ccc(-c2nc(-c3ccccc3)nc(-c3cccc(-c4ccc5ccc6oc7ccccc7c6c5c4)c3)n2)cc1]

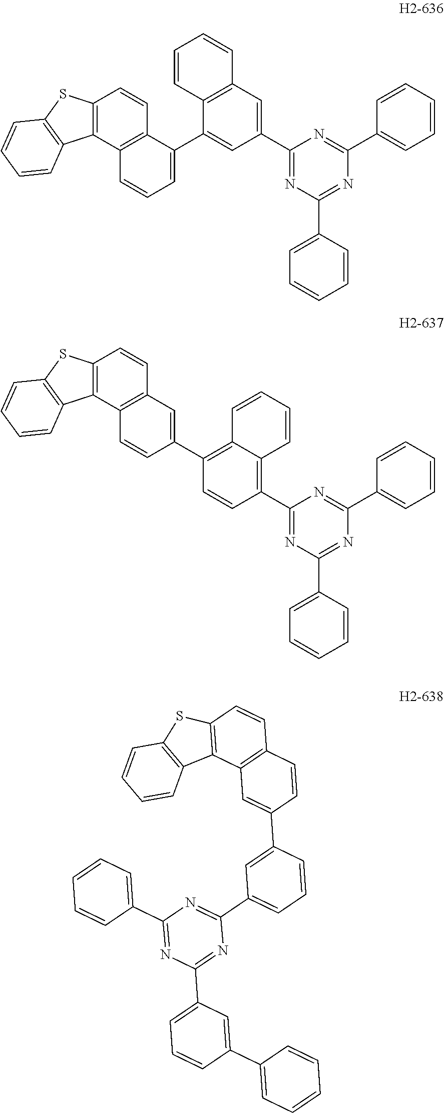 [SMILES: c1ccc(-c2nc(-c3ccccc3)nc(-c3cc(-c4cccc5c4ccc4sc6ccccc6c45)c4ccccc4c3)n2)cc1 ; c1ccc(-c2nc(-c3ccccc3)nc(-c3ccc(-c4ccc5c(ccc6sc7ccccc7c65)c4)c4ccccc34)n2)cc1 ; c1ccc(-c2cccc(-c3nc(-c4ccccc4)nc(-c4cccc(-c5ccc6ccc7sc8ccccc8c7c6c5)c4)n3)c2)cc1]

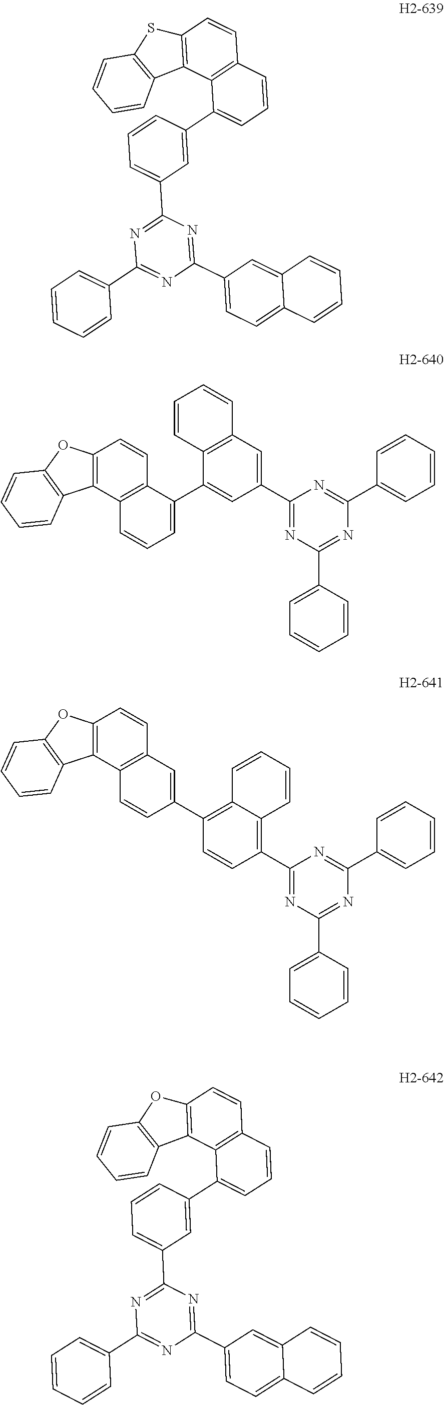 [SMILES: c1ccc(-c2nc(-c3cccc(-c4cccc5ccc6sc7ccccc7c6c45)c3)nc(-c3ccc4ccccc4c3)n2)cc1 ; c1ccc(-c2nc(-c3ccccc3)nc(-c3cc(-c4cccc5c4ccc4oc6ccccc6c45)c4ccccc4c3)n2)cc1 ; c1ccc(-c2nc(-c3ccccc3)nc(-c3ccc(-c4ccc5c(ccc6oc7ccccc7c65)c4)c4ccccc34)n2)cc1 ; c1ccc(-c2nc(-c3cccc(-c4cccc5ccc6oc7ccccc7c6c45)c3)nc(-c3ccc4ccccc4c3)n2)cc1]

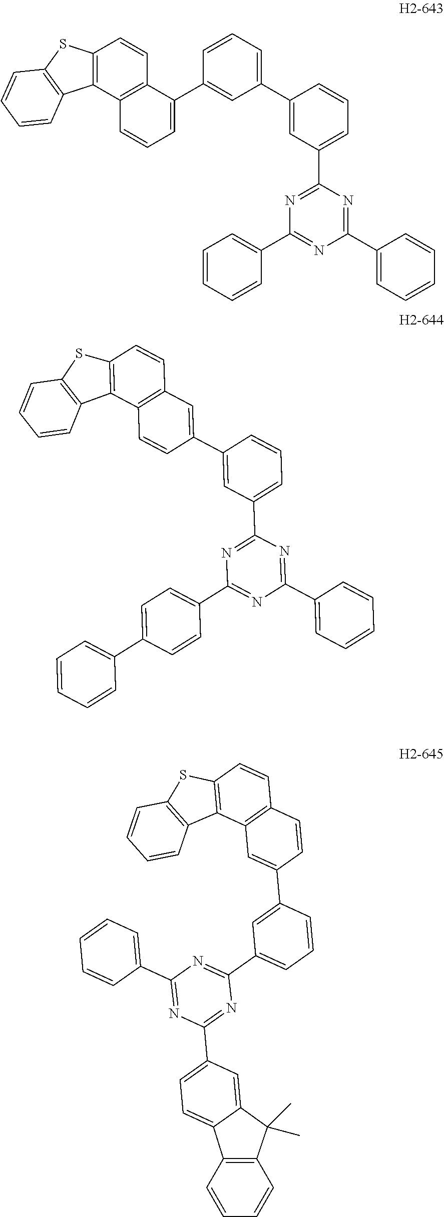 [SMILES: c1ccc(-c2nc(-c3ccccc3)nc(-c3cccc(-c4cccc(-c5cccc6c5ccc5sc7ccccc7c56)c4)c3)n2)cc1 ; c1ccc(-c2ccc(-c3nc(-c4ccccc4)nc(-c4cccc(-c5ccc6c(ccc7sc8ccccc8c76)c5)c4)n3)cc2)cc1 ; CC1(C)c2ccccc2-c2ccc(-c3nc(-c4ccccc4)nc(-c4cccc(-c5ccc6ccc7sc8ccccc8c7c6c5)c4)n3)cc21]

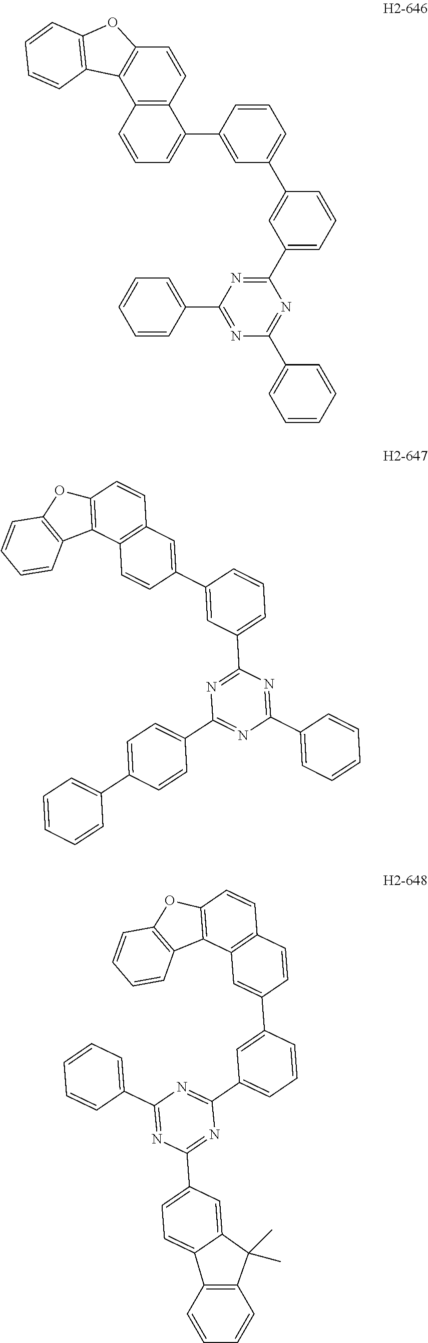 [SMILES: c1ccc(-c2nc(-c3ccccc3)nc(-c3cccc(-c4cccc(-c5cccc6c5ccc5oc7ccccc7c56)c4)c3)n2)cc1 ; c1ccc(-c2ccc(-c3nc(-c4ccccc4)nc(-c4cccc(-c5ccc6c(ccc7oc8ccccc8c76)c5)c4)n3)cc2)cc1 ; CC1(C)c2ccccc2-c2ccc(-c3nc(-c4ccccc4)nc(-c4cccc(-c5ccc6ccc7oc8ccccc8c7c6c5)c4)n3)cc21]

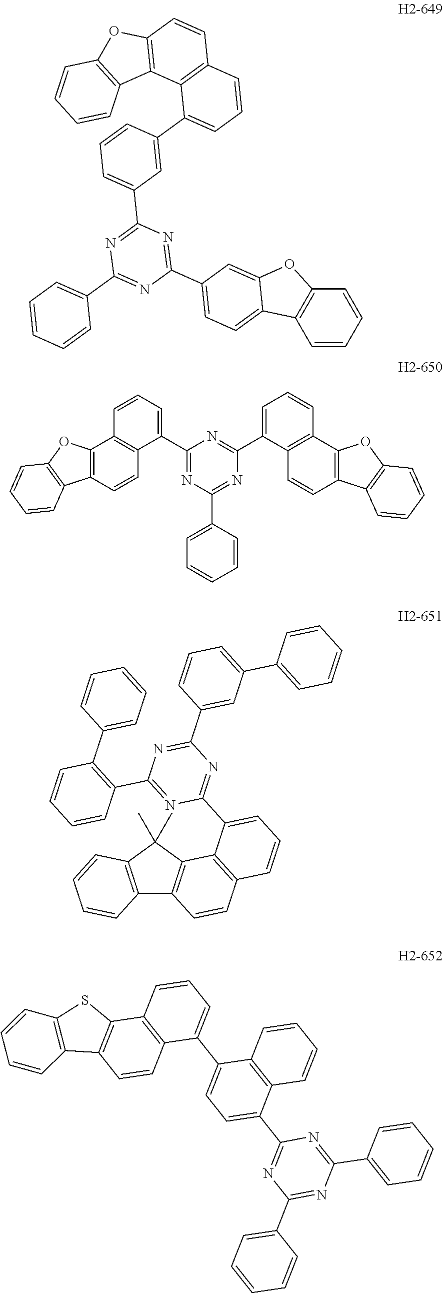 [SMILES: c1ccc(-c2nc(-c3cccc(-c4cccc5ccc6oc7ccccc7c6c45)c3)nc(-c3ccc4c(c3)oc3ccccc34)n2)cc1 ; c1ccc(-c2nc(-c3cccc4c3ccc3c5ccccc5oc43)nc(-c3cccc4c3ccc3c5ccccc5oc43)n2)cc1 ; CC1(C)c2ccccc2-c2ccc3cccc(-c4nc(-c5cccc(-c6ccccc6)c5)nc(-c5ccccc5-c5ccccc5)n4)c3c21 ; c1ccc(-c2nc(-c3ccccc3)nc(-c3ccc(-c4cccc5c4ccc4c6ccccc6sc54)c4ccccc34)n2)cc1]

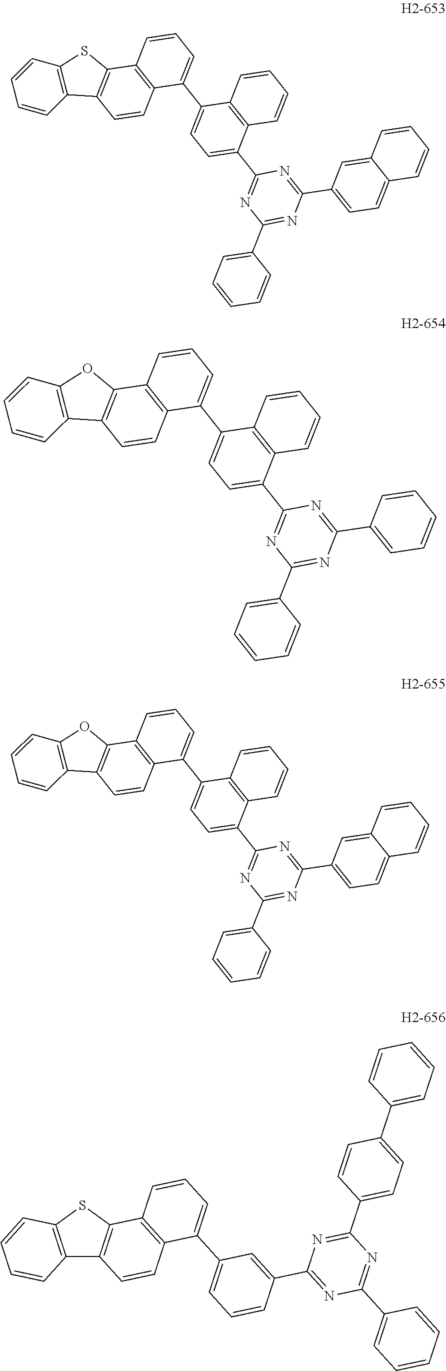 [SMILES: c1ccc(-c2nc(-c3ccc4ccccc4c3)nc(-c3ccc(-c4cccc5c4ccc4c6ccccc6sc54)c4ccccc34)n2)cc1 ; c1ccc(-c2nc(-c3ccccc3)nc(-c3ccc(-c4cccc5c4ccc4c6ccccc6oc54)c4ccccc34)n2)cc1 ; c1ccc(-c2nc(-c3ccc4ccccc4c3)nc(-c3ccc(-c4cccc5c4ccc4c6ccccc6oc54)c4ccccc34)n2)cc1 ; c1ccc(-c2ccc(-c3nc(-c4ccccc4)nc(-c4cccc(-c5cccc6c5ccc5c7ccccc7sc65)c4)n3)cc2)cc1]

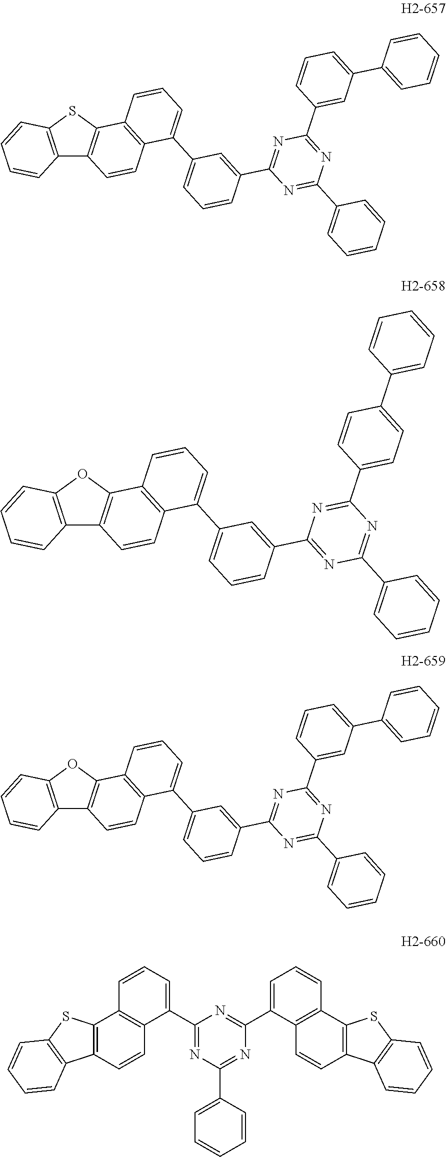 [SMILES: c1ccc(-c2cccc(-c3nc(-c4ccccc4)nc(-c4cccc(-c5cccc6c5ccc5c7ccccc7sc65)c4)n3)c2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2cccc(-c3nc(-c4ccccc4)nc(-c4cccc(-c5cccc6c5ccc5c7ccccc7oc65)c4)n3)c2)cc1 ; c1ccc(-c2nc(-c3cccc4c3ccc3c5ccccc5sc43)nc(-c3cccc4c3ccc3c5ccccc5sc43)n2)cc1]

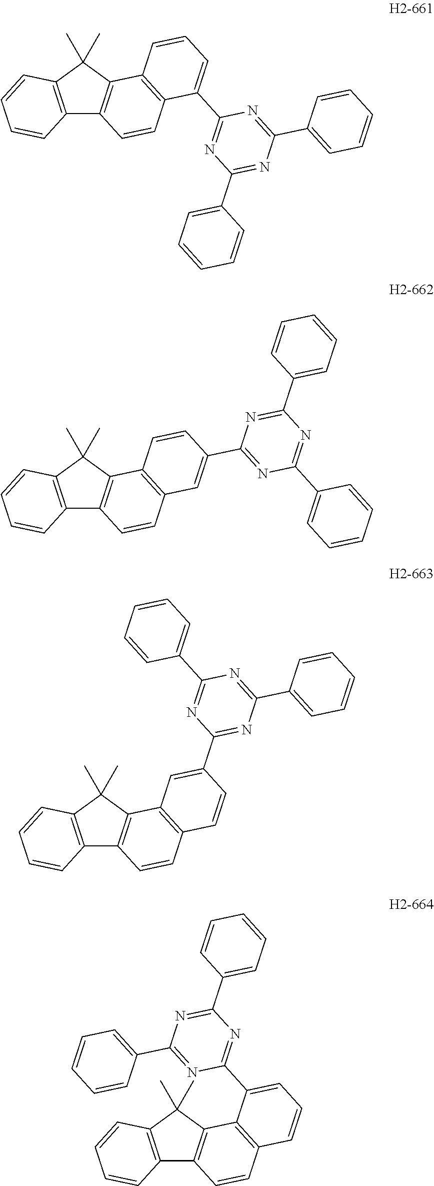 [SMILES: CC1(C)c2ccccc2-c2ccc3c(-c4nc(-c5ccccc5)nc(-c5ccccc5)n4)cccc3c21 ; CC1(C)c2ccccc2-c2ccc3cc(-c4nc(-c5ccccc5)nc(-c5ccccc5)n4)ccc3c21 ; CC1(C)c2ccccc2-c2ccc3ccc(-c4nc(-c5ccccc5)nc(-c5ccccc5)n4)cc3c21 ; CC1(C)c2ccccc2-c2ccc3cccc(-c4nc(-c5ccccc5)nc(-c5ccccc5)n4)c3c21]

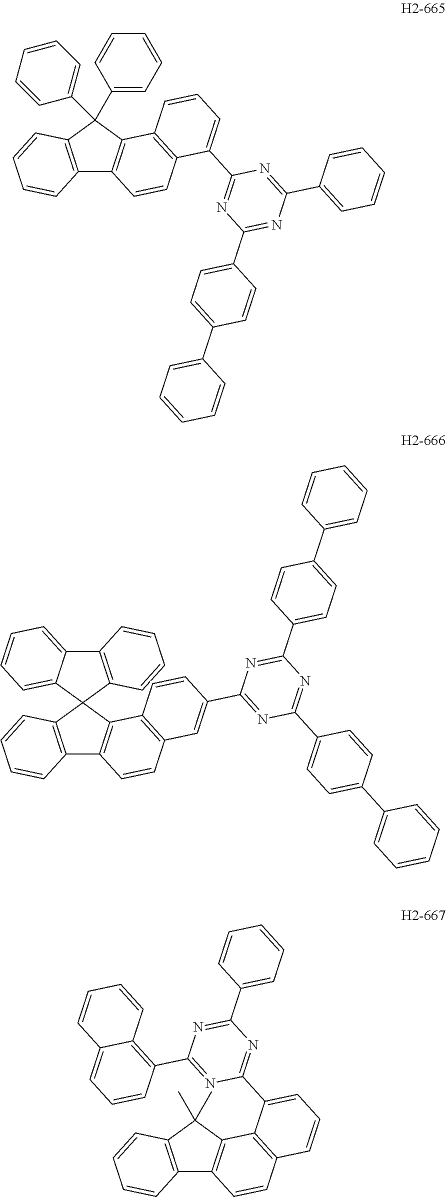 [SMILES: c1ccc(-c2ccc(-c3nc(-c4ccccc4)nc(-c4cccc5c6c(ccc45)-c4ccccc4C6(c4ccccc4)c4ccccc4)n3)cc2)cc1 ; c1ccc(-c2ccc(-c3nc(-c4ccc(-c5ccccc5)cc4)nc(-c4ccc5c6c(ccc5c4)-c4ccccc4C64c5ccccc5-c5ccccc54)n3)cc2)cc1 ; CC1(C)c2ccccc2-c2ccc3cccc(-c4nc(-c5ccccc5)nc(-c5cccc6ccccc56)n4)c3c21]

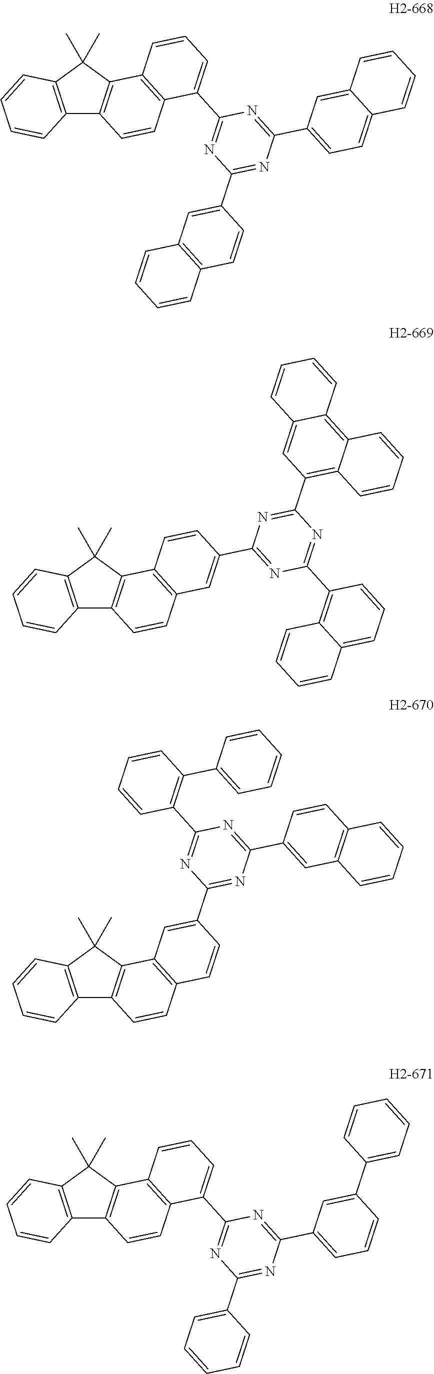 [SMILES: CC1(C)c2ccccc2-c2ccc3c(-c4nc(-c5ccc6ccccc6c5)nc(-c5ccc6ccccc6c5)n4)cccc3c21 ; CC1(C)c2ccccc2-c2ccc3cc(-c4nc(-c5cccc6ccccc56)nc(-c5cc6ccccc6c6ccccc56)n4)ccc3c21 ; CC1(C)c2ccccc2-c2ccc3ccc(-c4nc(-c5ccc6ccccc6c5)nc(-c5ccccc5-c5ccccc5)n4)cc3c21 ; CC1(C)c2ccccc2-c2ccc3c(-c4nc(-c5ccccc5)nc(-c5cccc(-c6ccccc6)c5)n4)cccc3c21]

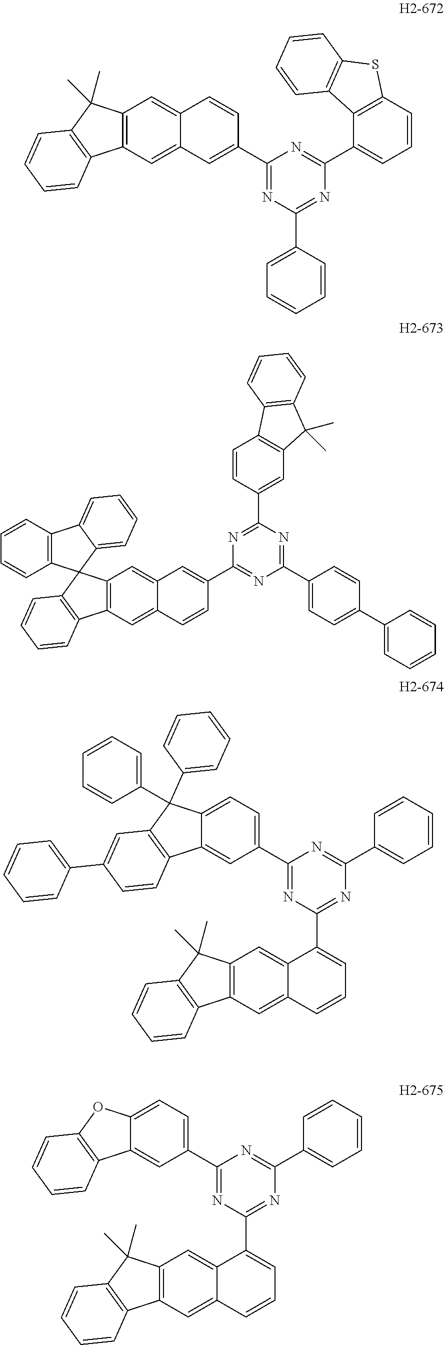

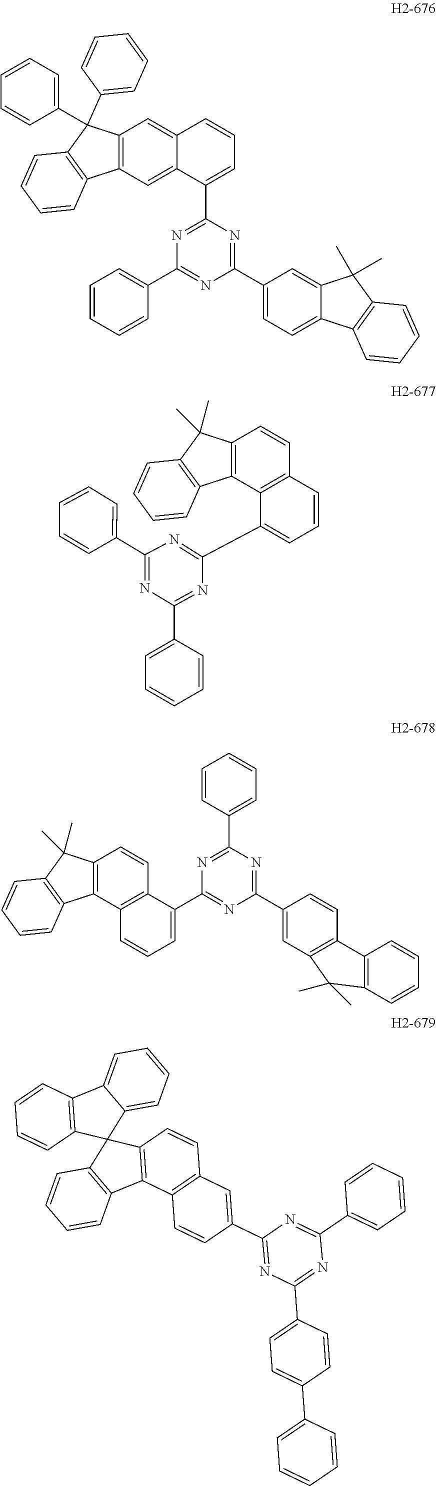 [SMILES: CC1(C)c2ccccc2-c2ccc(-c3nc(-c4ccccc4)nc(-c4cccc5cc6c(cc45)-c4ccccc4C6(c4ccccc4)c4ccccc4)n3)cc21 ; CC1(C)c2ccccc2-c2c1ccc1cccc(-c3nc(-c4ccccc4)nc(-c4ccccc4)n3)c21 ; CC1(C)c2ccccc2-c2ccc(-c3nc(-c4ccccc4)nc(-c4cccc5c6c(ccc45)C(C)(C)c4ccccc4-6)n3)cc21 ; c1ccc(-c2ccc(-c3nc(-c4ccccc4)nc(-c4ccc5c6c(ccc5c4)C4(c5ccccc5-c5ccccc54)c4ccccc4-6)n3)cc2)cc1]

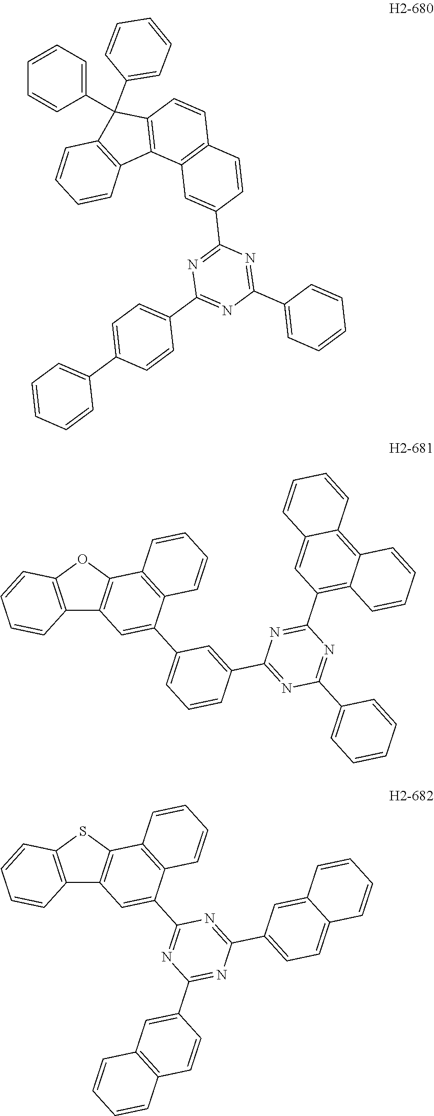 [SMILES: c1ccc(-c2ccc(-c3nc(-c4ccccc4)nc(-c4ccc5ccc6c(c5c4)-c4ccccc4C6(c4ccccc4)c4ccccc4)n3)cc2)cc1 ; c1ccc(-c2nc(-c3cccc(-c4cc5c6ccccc6oc5c5ccccc45)c3)nc(-c3cc4ccccc4c4ccccc34)n2)cc1 ; c1ccc2cc(-c3nc(-c4ccc5ccccc5c4)nc(-c4cc5c6ccccc6sc5c5ccccc45)n3)ccc2c1]

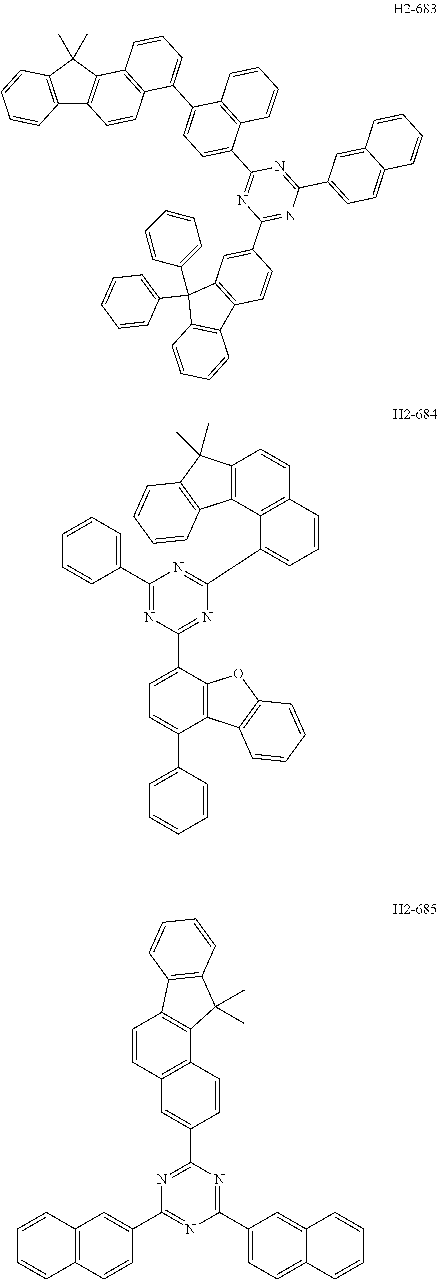

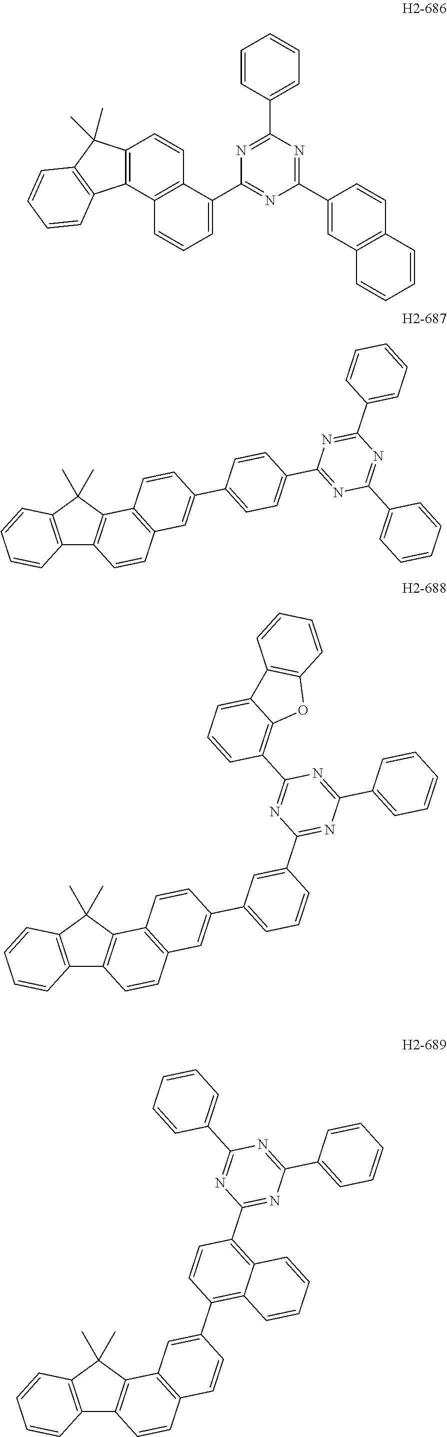

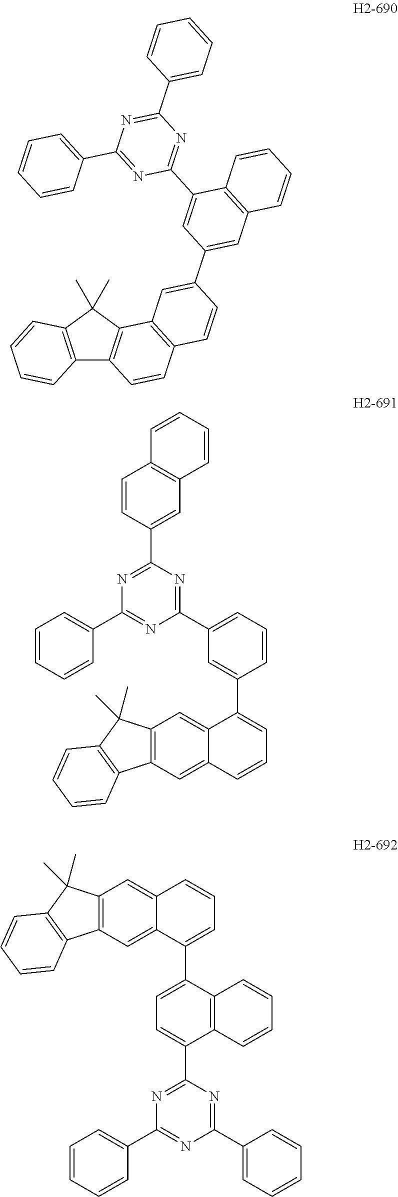

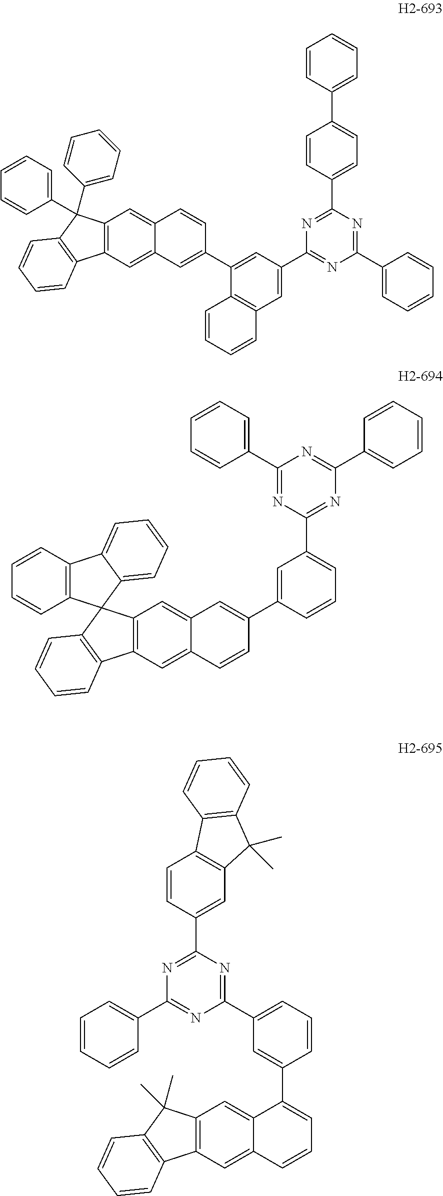 [SMILES: c1ccc(-c2ccc(-c3nc(-c4ccccc4)nc(-c4cc(-c5ccc6cc7c(cc6c5)-c5ccccc5C7(c5ccccc5)c5ccccc5)c5ccccc5c4)n3)cc2)cc1 ; c1ccc(-c2nc(-c3ccccc3)nc(-c3cccc(-c4ccc5cc6c(cc5c4)C4(c5ccccc5-c5ccccc54)c4ccccc4-6)c3)n2)cc1 ; CC1(C)c2ccccc2-c2ccc(-c3nc(-c4ccccc4)nc(-c4cccc(-c5cccc6cc7c(cc56)C(C)(C)c5ccccc5-7)c4)n3)cc21]

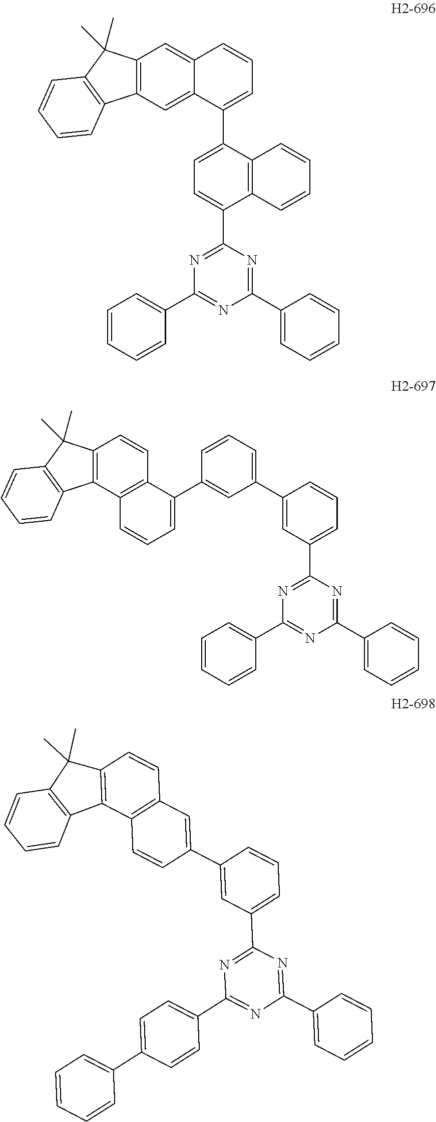 [SMILES: CC1(C)c2ccccc2-c2cc3c(-c4ccc(-c5nc(-c6ccccc6)nc(-c6ccccc6)n5)c5ccccc45)cccc3cc21 ; CC1(C)c2ccccc2-c2c1ccc1c(-c3cccc(-c4cccc(-c5nc(-c6ccccc6)nc(-c6ccccc6)n5)c4)c3)cccc21 ; CC1(C)c2ccccc2-c2c1ccc1cc(-c3cccc(-c4nc(-c5ccccc5)nc(-c5ccc(-c6ccccc6)cc5)n4)c3)ccc21]

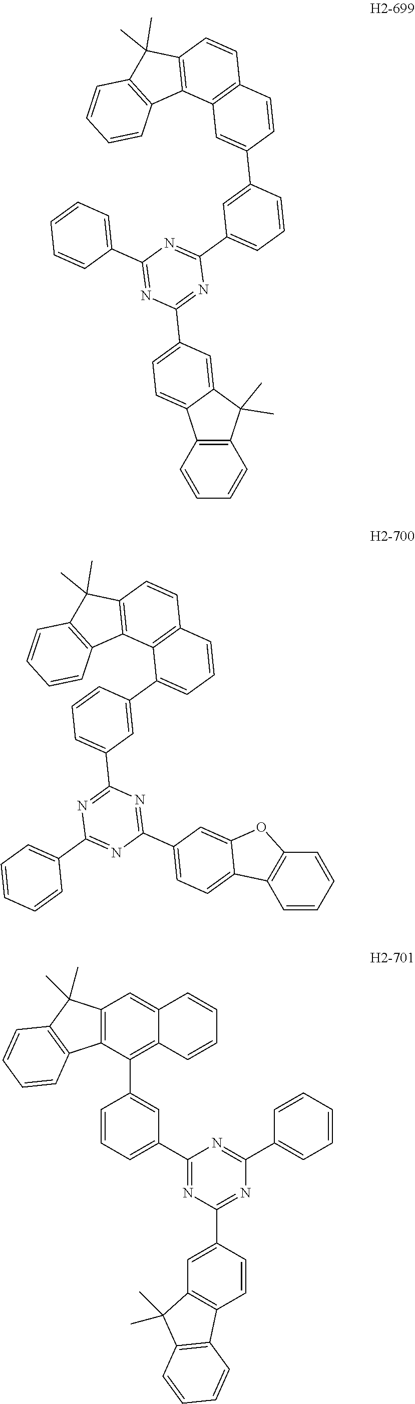 [SMILES: CC1(C)c2ccccc2-c2ccc(-c3nc(-c4ccccc4)nc(-c4cccc(-c5ccc6ccc7c(c6c5)-c5ccccc5C7(C)C)c4)n3)cc21 ; CC1(C)c2ccccc2-c2c1ccc1cccc(-c3cccc(-c4nc(-c5ccccc5)nc(-c5ccc6c(c5)oc5ccccc56)n4)c3)c21 ; CC1(C)c2ccccc2-c2ccc(-c3nc(-c4ccccc4)nc(-c4cccc(-c5c6c(cc7ccccc57)C(C)(C)c5ccccc5-6)c4)n3)cc21]

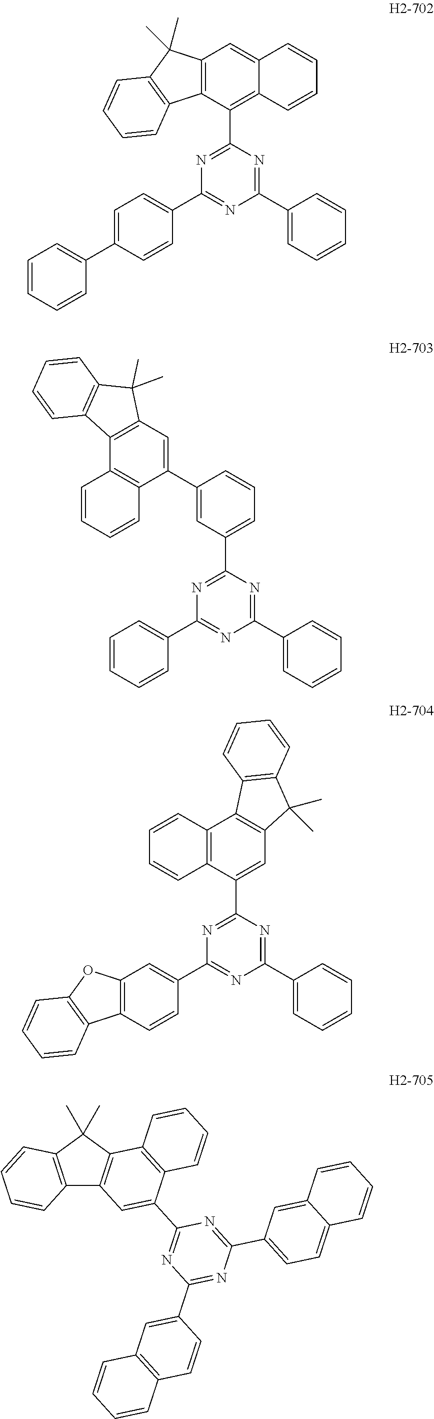 [SMILES: CC1(C)c2ccccc2-c2c1cc1ccccc1c2-c1nc(-c2ccccc2)nc(-c2ccc(-c3ccccc3)cc2)n1 ; CC1(C)c2ccccc2-c2c1cc(-c1cccc(-c3nc(-c4ccccc4)nc(-c4ccccc4)n3)c1)c1ccccc21 ; CC1(C)c2ccccc2-c2c1cc(-c1nc(-c3ccccc3)nc(-c3ccc4c(c3)oc3ccccc34)n1)c1ccccc21 ; CC1(C)c2ccccc2-c2cc(-c3nc(-c4ccc5ccccc5c4)nc(-c4ccc5ccccc5c4)n3)c3ccccc3c21]

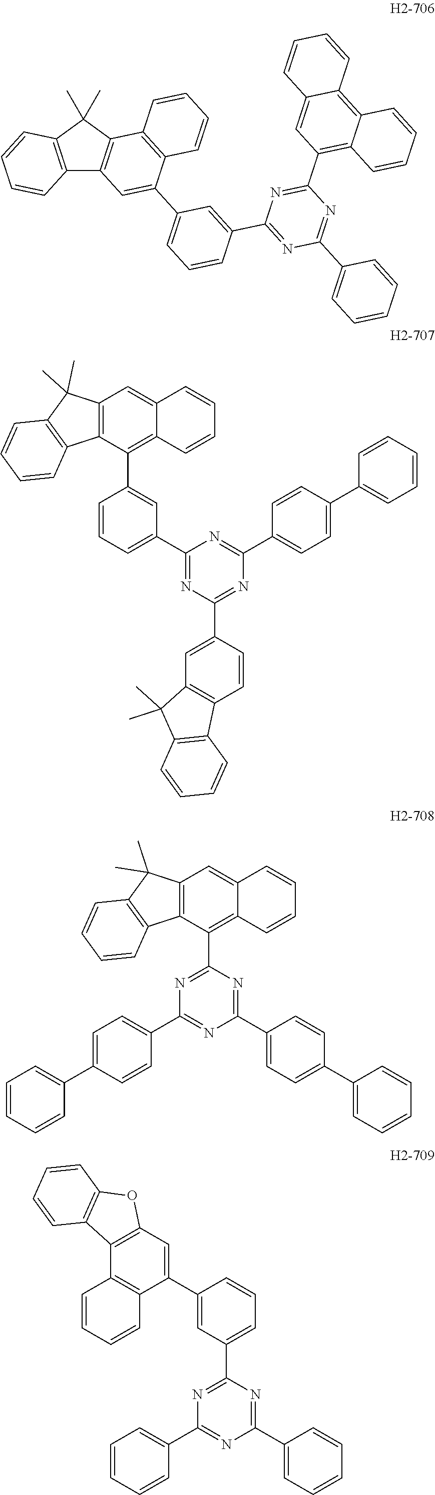

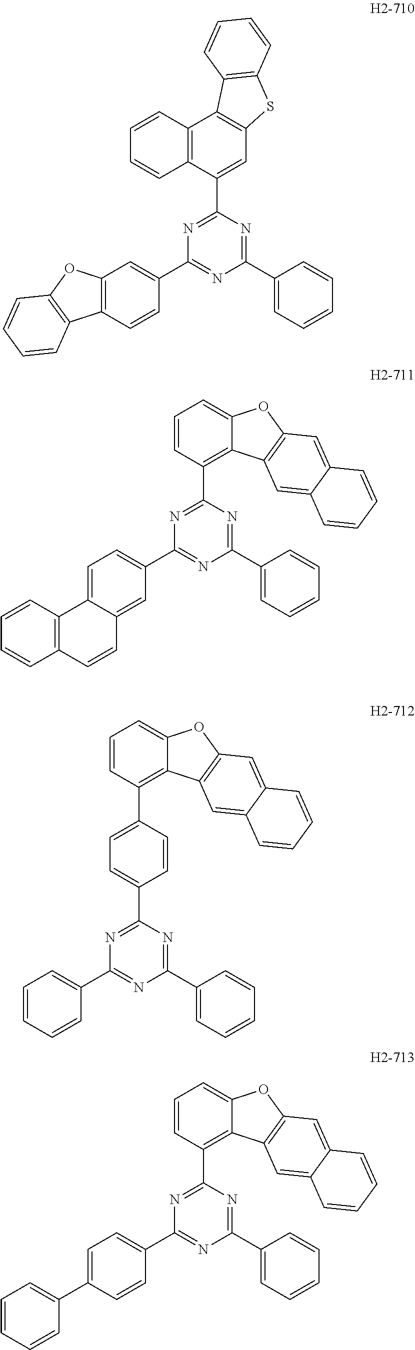 [SMILES: c1ccc(-c2nc(-c3ccc4c(c3)oc3ccccc34)nc(-c3cc4sc5ccccc5c4c4ccccc34)n2)cc1 ; c1ccc(-c2nc(-c3ccc4c(ccc5ccccc54)c3)nc(-c3cccc4oc5cc6ccccc6cc5c34)n2)cc1 ; c1ccc(-c2nc(-c3ccccc3)nc(-c3ccc(-c4cccc5oc6cc7ccccc7cc6c45)cc3)n2)cc1 ; c1ccc(-c2ccc(-c3nc(-c4ccccc4)nc(-c4cccc5oc6cc7ccccc7cc6c45)n3)cc2)cc1]

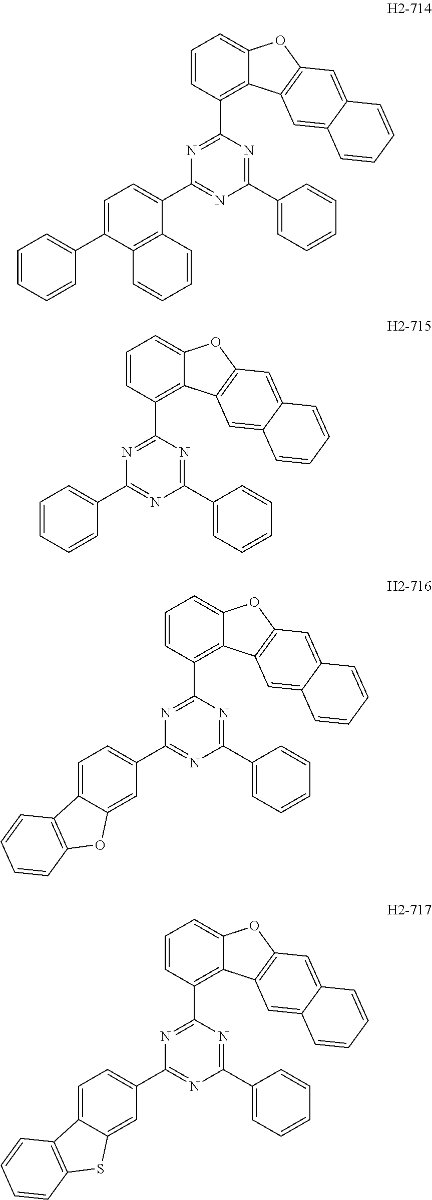

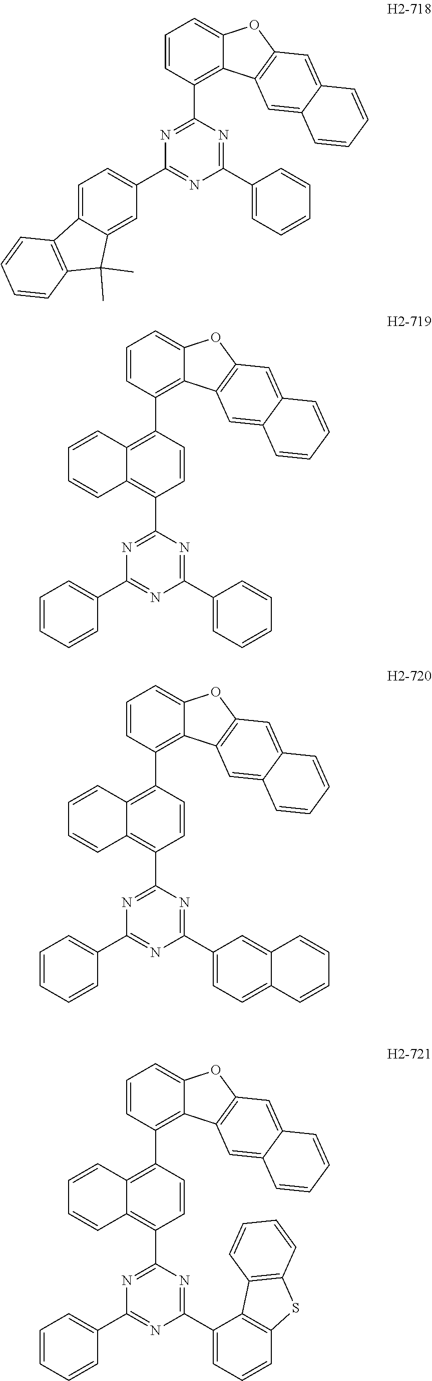 [SMILES: CC1(C)c2ccccc2-c2ccc(-c3nc(-c4ccccc4)nc(-c4cccc5oc6cc7ccccc7cc6c45)n3)cc21 ; c1ccc(-c2nc(-c3ccccc3)nc(-c3ccc(-c4cccc5oc6cc7ccccc7cc6c45)c4ccccc34)n2)cc1 ; c1ccc(-c2nc(-c3ccc4ccccc4c3)nc(-c3ccc(-c4cccc5oc6cc7ccccc7cc6c45)c4ccccc34)n2)cc1 ; c1ccc(-c2nc(-c3ccc(-c4cccc5oc6cc7ccccc7cc6c45)c4ccccc34)nc(-c3cccc4sc5ccccc5c34)n2)cc1]

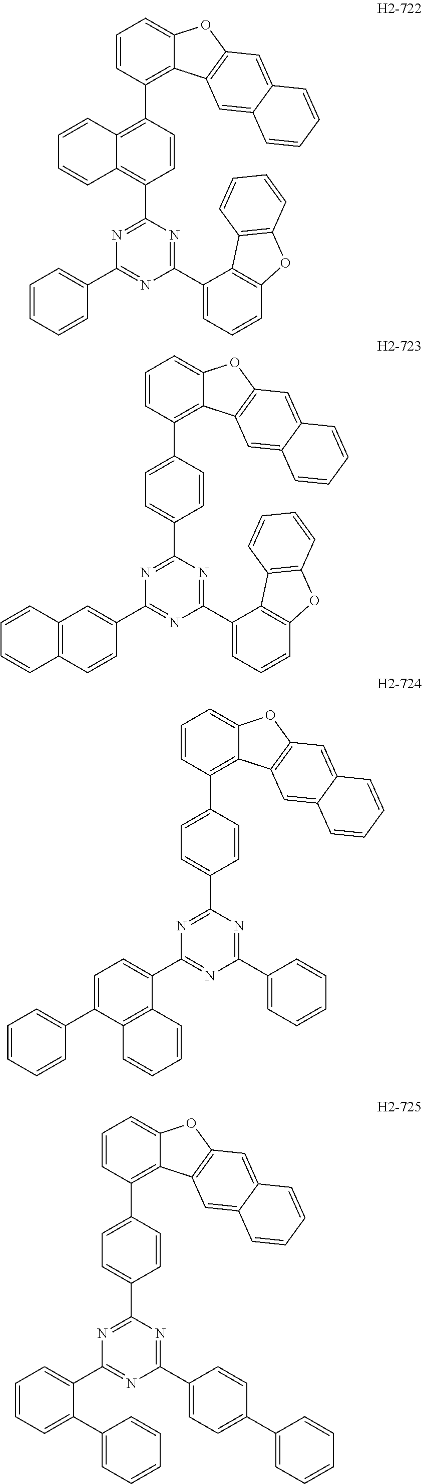

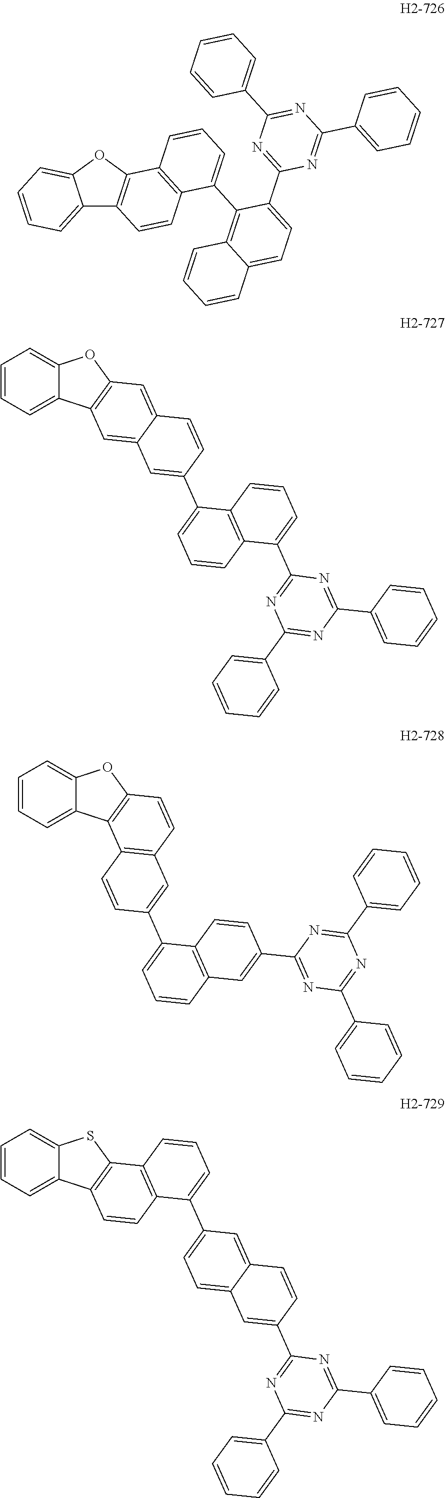 [SMILES: c1ccc(-c2nc(-c3ccccc3)nc(-c3ccc4ccccc4c3-c3cccc4c3ccc3c5ccccc5oc43)n2)cc1 ; c1ccc(-c2nc(-c3ccccc3)nc(-c3cccc4c(-c5ccc6cc7oc8ccccc8c7cc6c5)cccc34)n2)cc1 ; c1ccc(-c2nc(-c3ccccc3)nc(-c3ccc4c(-c5ccc6c(ccc7oc8ccccc8c76)c5)cccc4c3)n2)cc1 ; c1ccc(-c2nc(-c3ccccc3)nc(-c3ccc4cc(-c5cccc6c5ccc5c7ccccc7sc65)ccc4c3)n2)cc1]

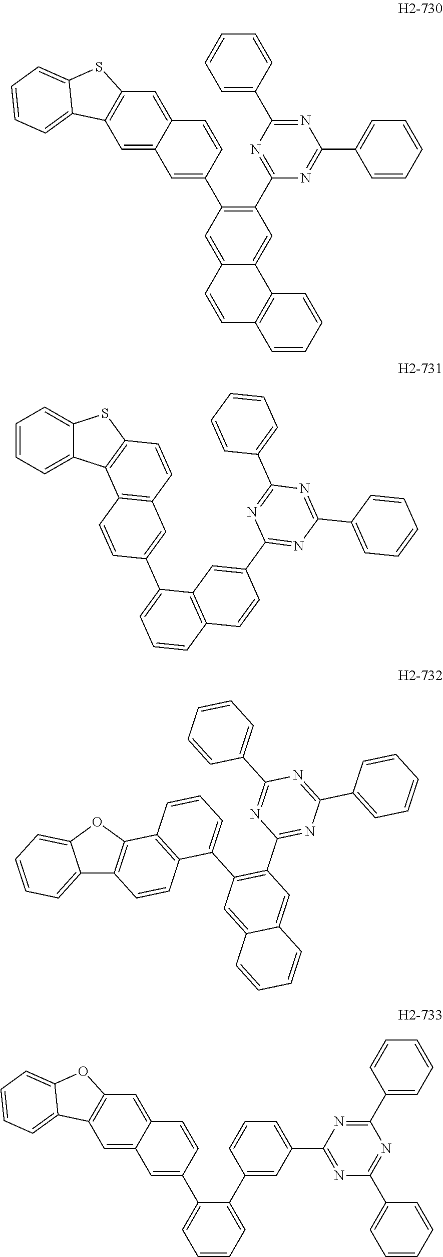

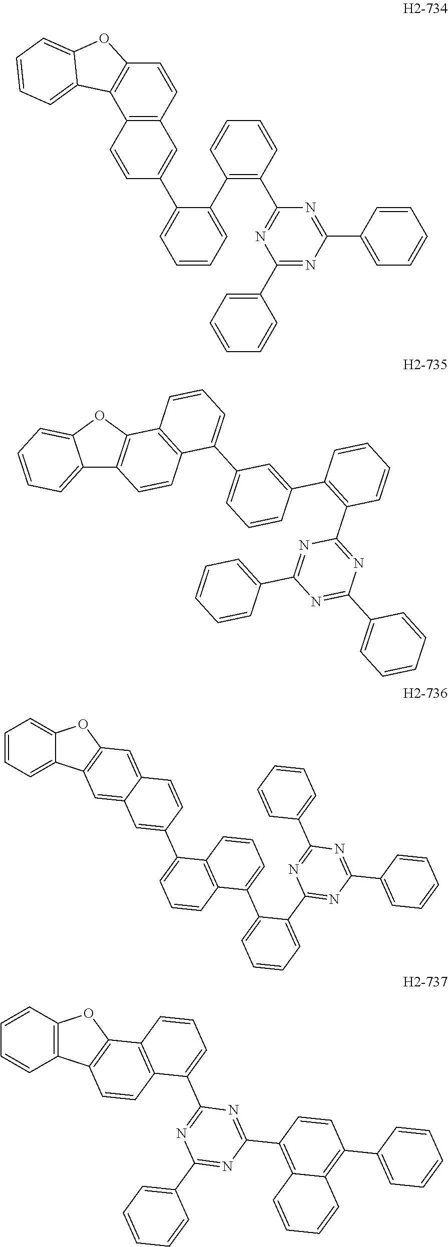

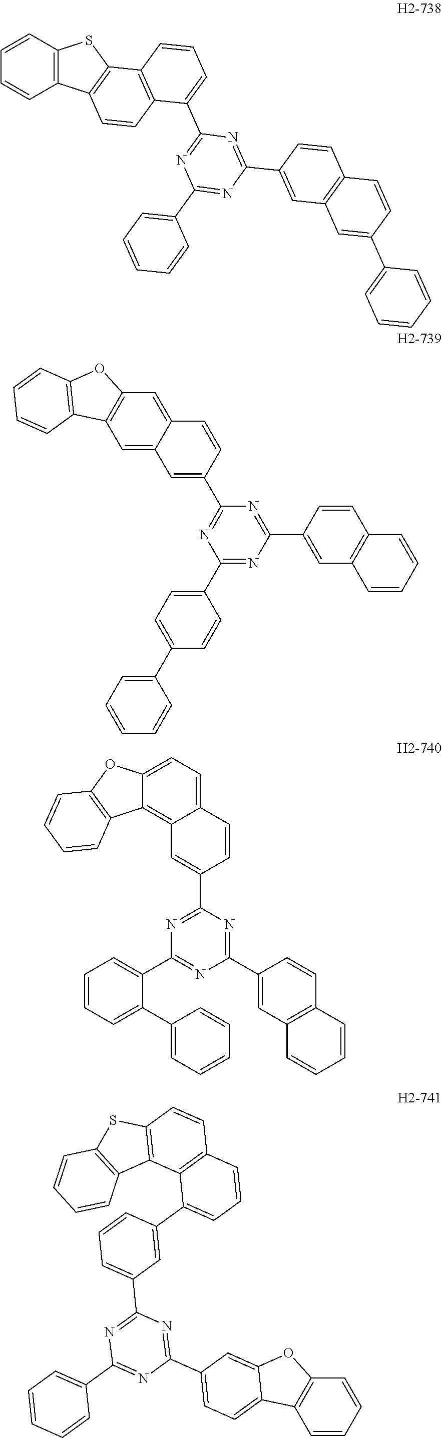

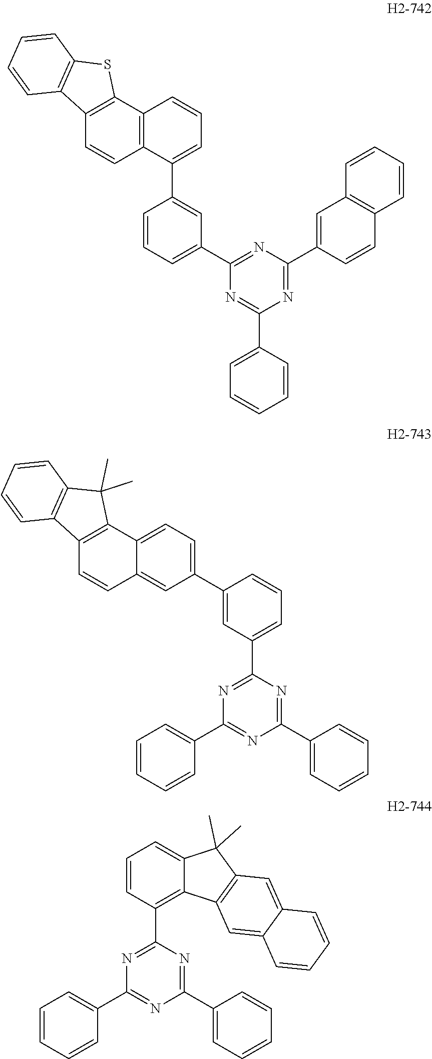

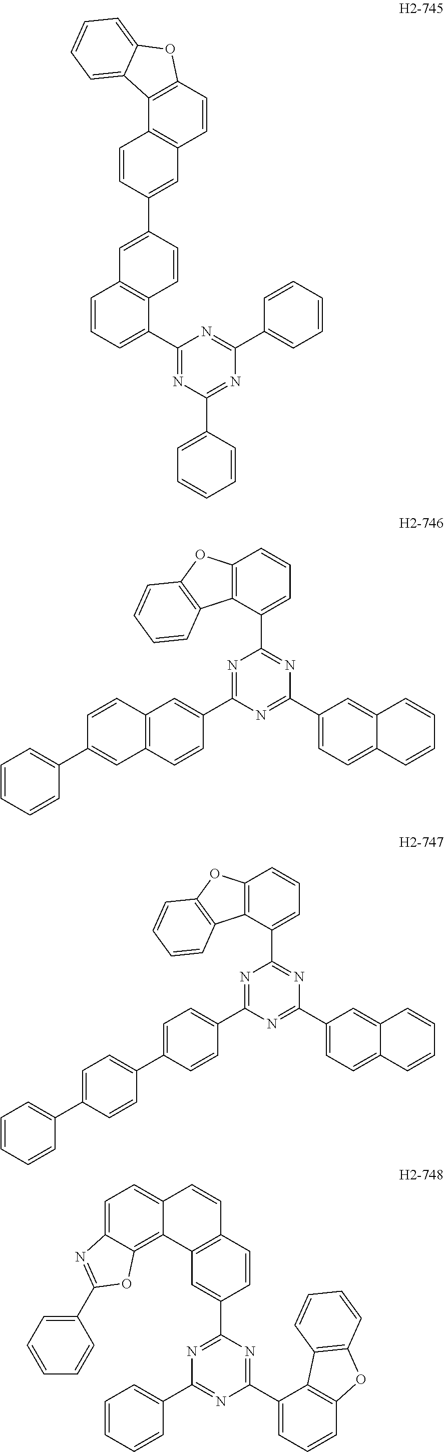 [SMILES: c1ccc(-c2nc(-c3ccccc3)nc(-c3cccc4cc(-c5ccc6c(ccc7oc8ccccc8c76)c5)ccc34)n2)cc1 ; c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc(-c2nc(-c3ccc4ccc5ccc6nc(-c7ccccc7)oc6c5c4c3)nc(-c3cccc4oc5ccccc5c34)n2)cc1]

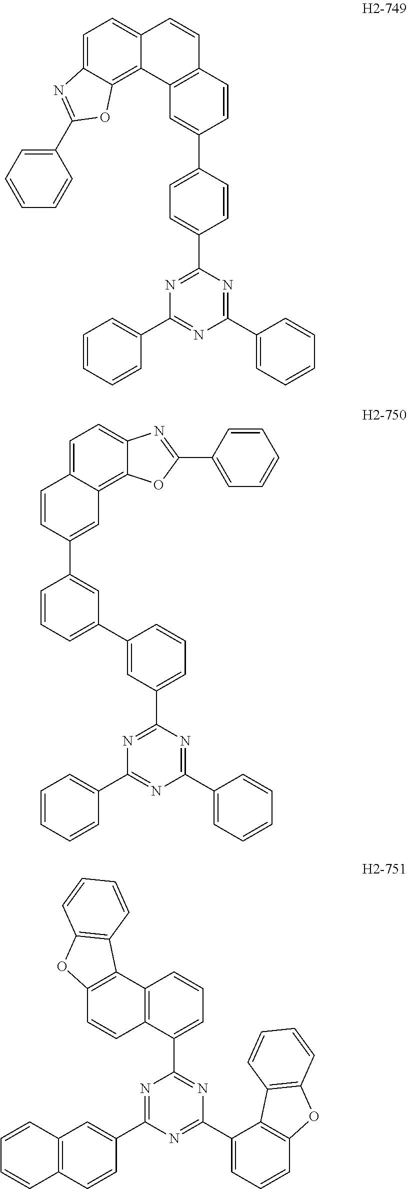

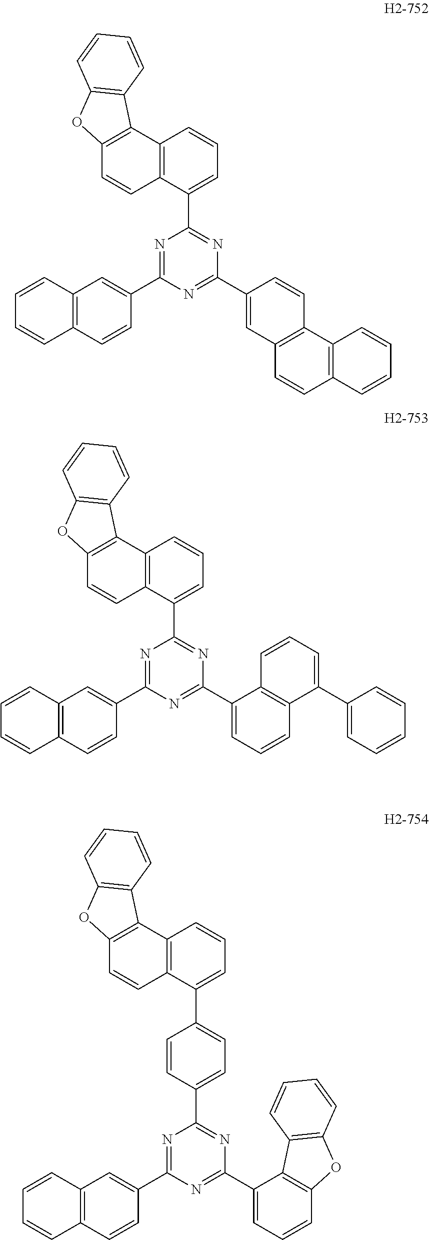 [SMILES: c1ccc2cc(-c3nc(-c4ccc5c(ccc6ccccc65)c4)nc(-c4cccc5c4ccc4oc6ccccc6c45)n3)ccc2c1 ; c1ccc(-c2cccc3c(-c4nc(-c5ccc6ccccc6c5)nc(-c5cccc6c5ccc5oc7ccccc7c56)n4)cccc23)cc1 ; c1ccc2cc(-c3nc(-c4ccc(-c5cccc6c5ccc5oc7ccccc7c56)cc4)nc(-c4cccc5oc6ccccc6c45)n3)ccc2c1]

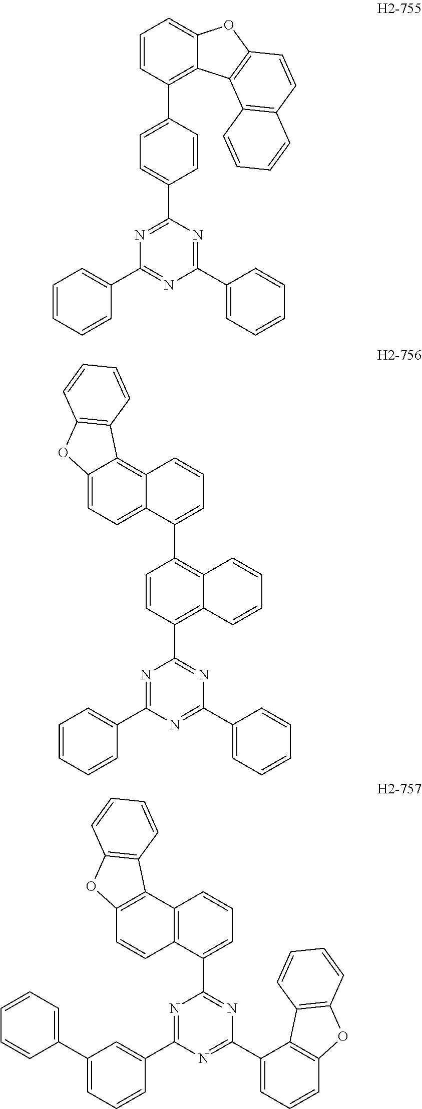

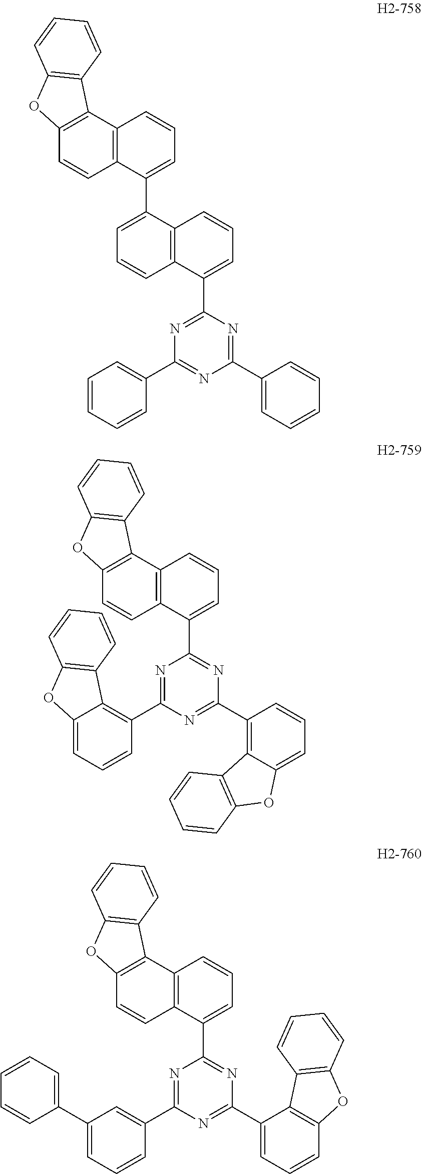

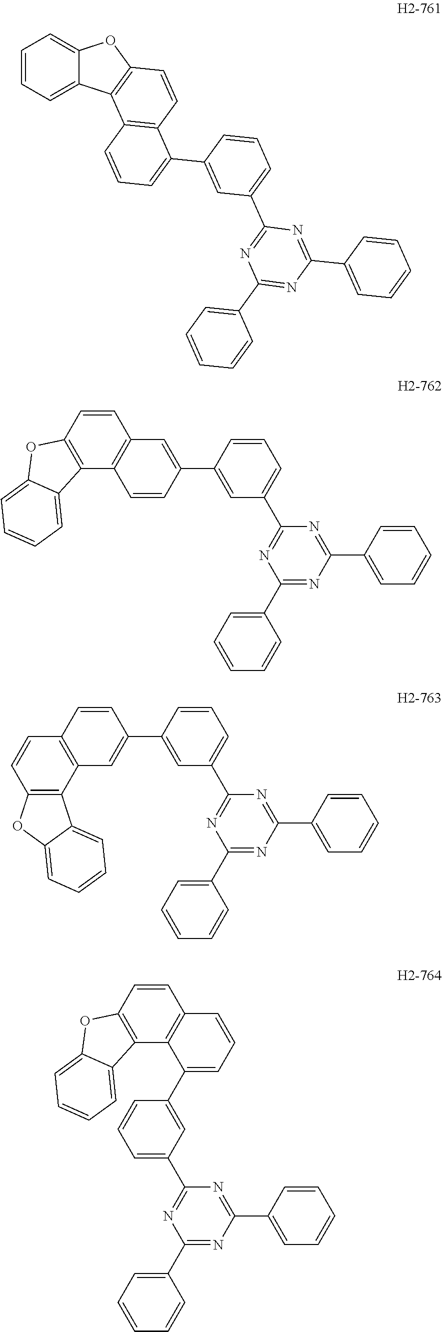

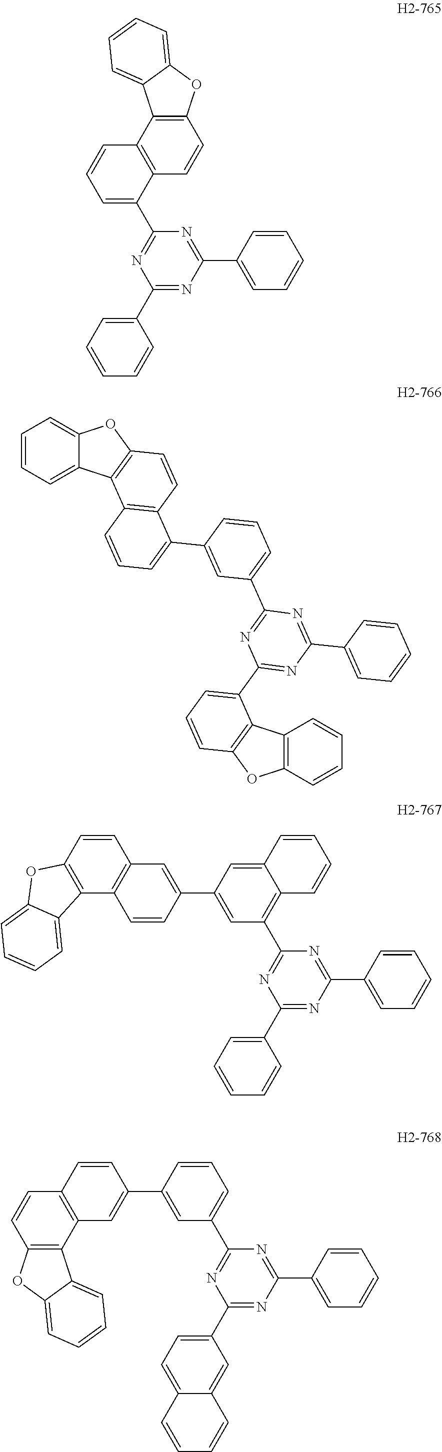 [SMILES: c1ccc(-c2nc(-c3ccccc3)nc(-c3cccc4c3ccc3oc5ccccc5c34)n2)cc1 ; c1ccc(-c2nc(-c3cccc(-c4cccc5c4ccc4oc6ccccc6c45)c3)nc(-c3cccc4oc5ccccc5c34)n2)cc1 ; c1ccc(-c2nc(-c3ccccc3)nc(-c3cc(-c4ccc5c(ccc6oc7ccccc7c65)c4)cc4ccccc34)n2)cc1 ; c1ccc(-c2nc(-c3cccc(-c4ccc5ccc6oc7ccccc7c6c5c4)c3)nc(-c3ccc4ccccc4c3)n2)cc1]

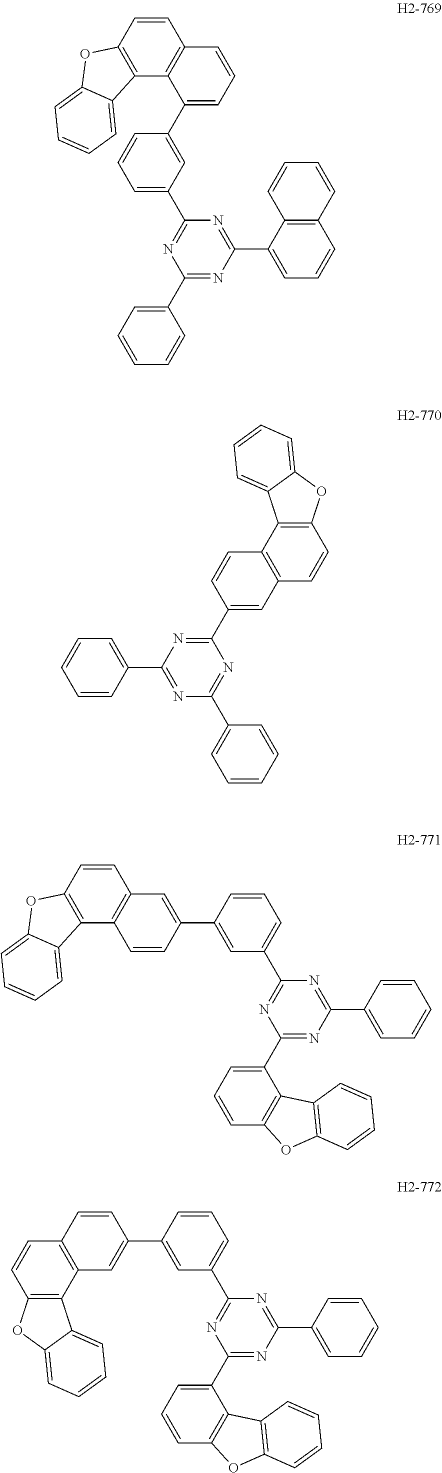 [SMILES: c1ccc(-c2nc(-c3cccc(-c4cccc5ccc6oc7ccccc7c6c45)c3)nc(-c3cccc4ccccc34)n2)cc1 ; c1ccc(-c2nc(-c3ccccc3)nc(-c3ccc4c(ccc5oc6ccccc6c54)c3)n2)cc1 ; c1ccc(-c2nc(-c3cccc(-c4ccc5c(ccc6oc7ccccc7c65)c4)c3)nc(-c3cccc4oc5ccccc5c34)n2)cc1 ; c1ccc(-c2nc(-c3cccc(-c4ccc5ccc6oc7ccccc7c6c5c4)c3)nc(-c3cccc4oc5ccccc5c34)n2)cc1]

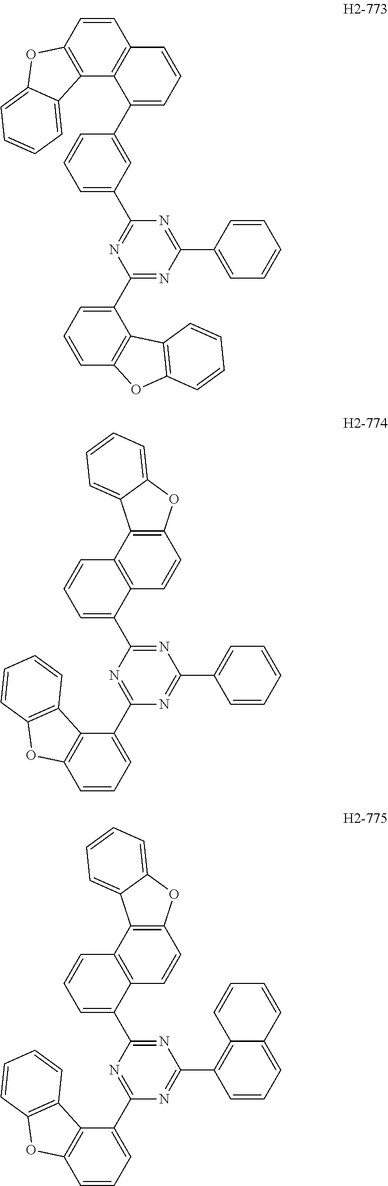

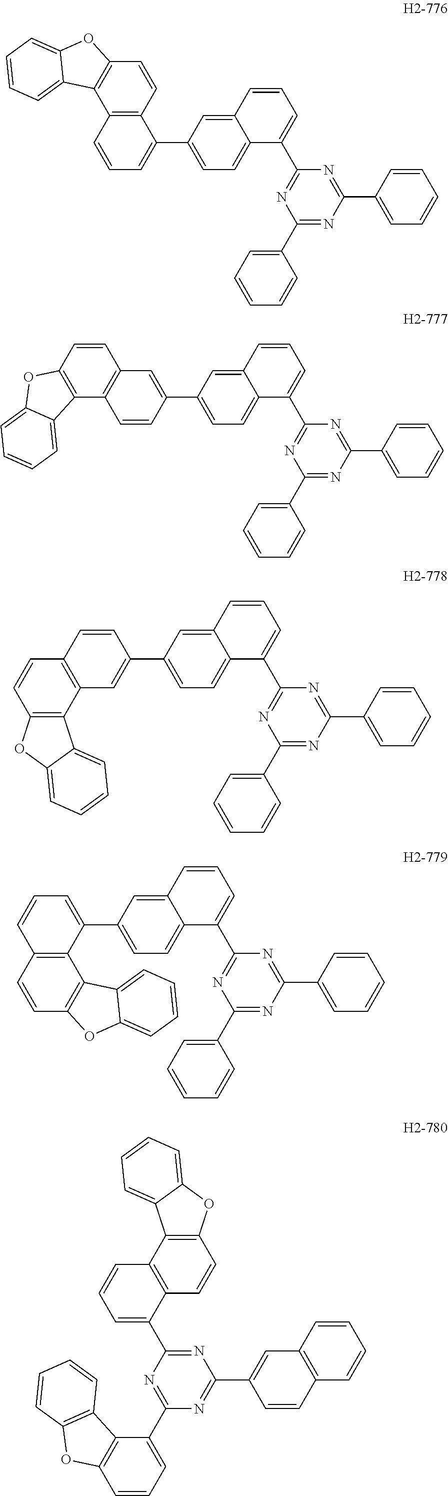

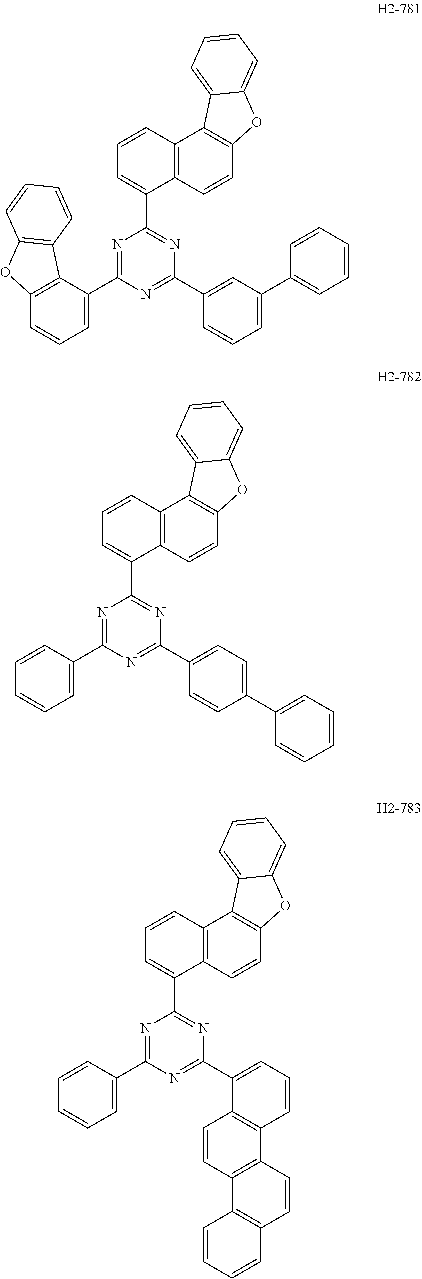 [SMILES: c1ccc(-c2cccc(-c3nc(-c4cccc5c4ccc4oc6ccccc6c45)nc(-c4cccc5oc6ccccc6c45)n3)c2)cc1 ; c1ccc(-c2ccc(-c3nc(-c4ccccc4)nc(-c4cccc5c4ccc4oc6ccccc6c45)n3)cc2)cc1 ; c1ccc(-c2nc(-c3cccc4c3ccc3c5ccccc5ccc43)nc(-c3cccc4c3ccc3oc5ccccc5c34)n2)cc1]

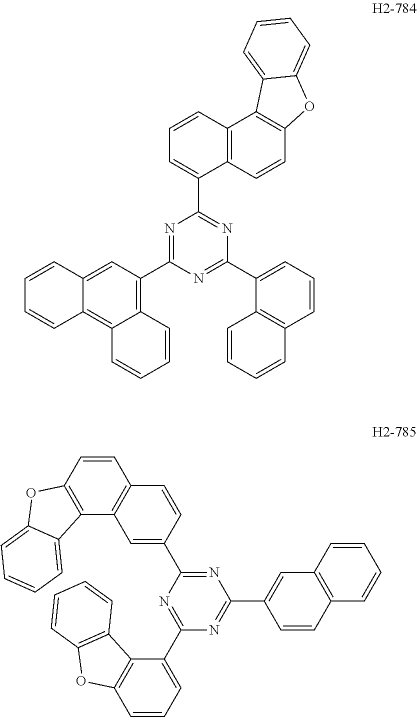 [SMILES: c1ccc2c(-c3nc(-c4cc5ccccc5c5ccccc45)nc(-c4cccc5c4ccc4oc6ccccc6c45)n3)cccc2c1 ; c1ccc2cc(-c3nc(-c4ccc5ccc6oc7ccccc7c6c5c4)nc(-c4cccc5oc6ccccc6c45)n3)ccc2c1]

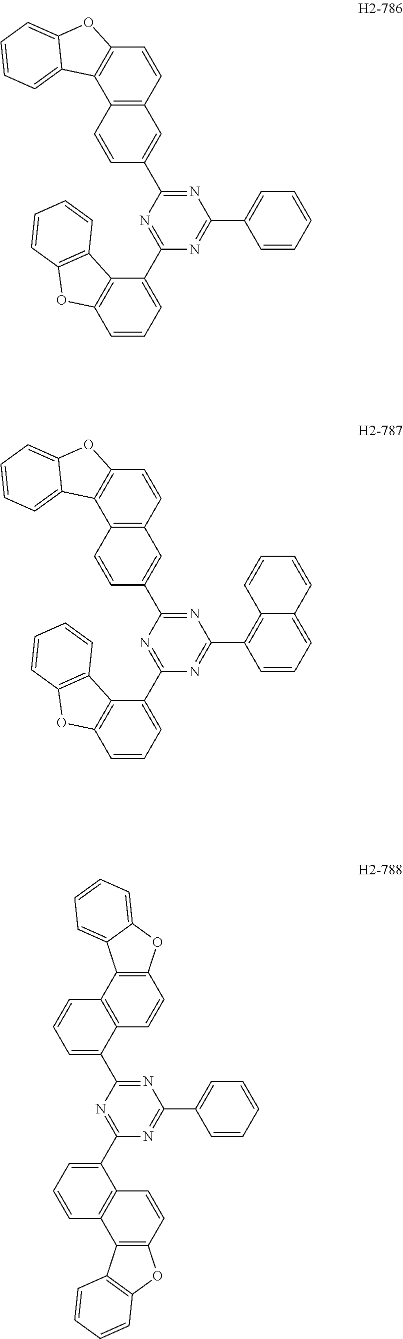 [SMILES: c1ccc(-c2nc(-c3ccc4c(ccc5oc6ccccc6c54)c3)nc(-c3cccc4oc5ccccc5c34)n2)cc1 ; c1ccc2c(-c3nc(-c4ccc5c(ccc6oc7ccccc7c65)c4)nc(-c4cccc5oc6ccccc6c45)n3)cccc2c1 ; c1ccc(-c2nc(-c3cccc4c3ccc3oc5ccccc5c34)nc(-c3cccc4c3ccc3oc5ccccc5c34)n2)cc1]

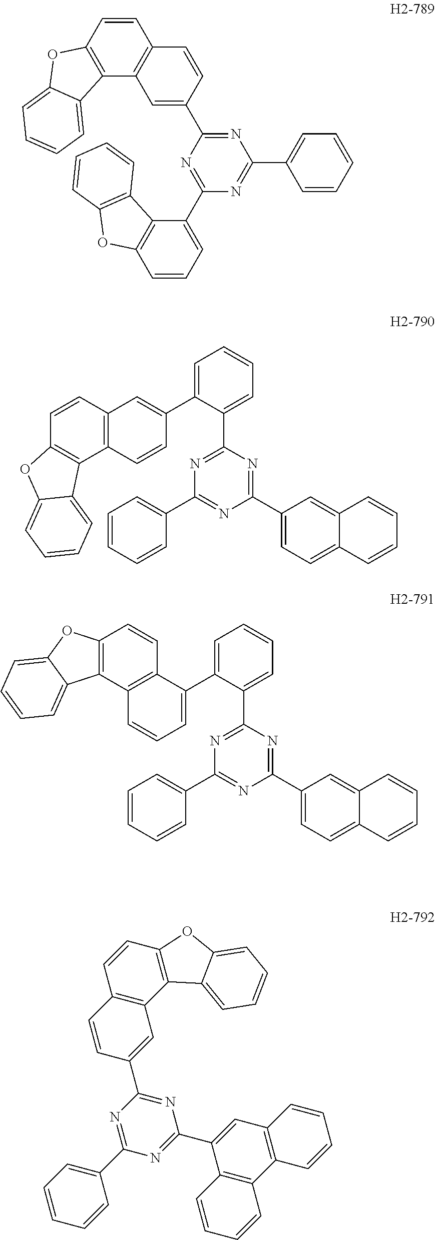 [SMILES: c1ccc(-c2nc(-c3ccc4ccc5oc6ccccc6c5c4c3)nc(-c3cccc4oc5ccccc5c34)n2)cc1 ; c1ccc(-c2nc(-c3ccc4ccccc4c3)nc(-c3ccccc3-c3ccc4c(ccc5oc6ccccc6c54)c3)n2)cc1 ; c1ccc(-c2nc(-c3ccc4ccccc4c3)nc(-c3ccccc3-c3cccc4c3ccc3oc5ccccc5c34)n2)cc1 ; c1ccc(-c2nc(-c3ccc4ccc5oc6ccccc6c5c4c3)nc(-c3cc4ccccc4c4ccccc34)n2)cc1]

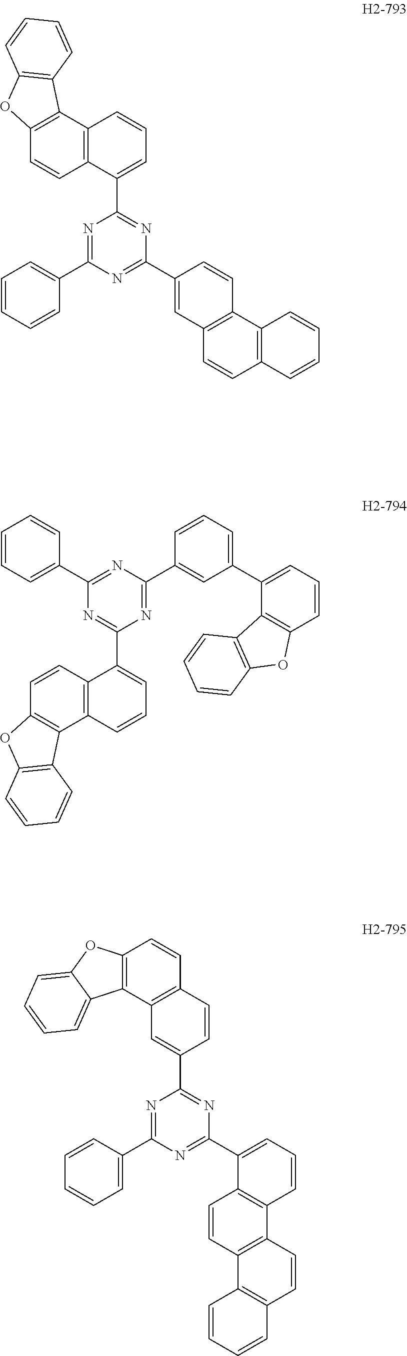 [SMILES: c1ccc(-c2nc(-c3ccc4c(ccc5ccccc54)c3)nc(-c3cccc4c3ccc3oc5ccccc5c34)n2)cc1 ; c1ccc(-c2nc(-c3cccc(-c4cccc5oc6ccccc6c45)c3)nc(-c3cccc4c3ccc3oc5ccccc5c34)n2)cc1 ; c1ccc(-c2nc(-c3ccc4ccc5oc6ccccc6c5c4c3)nc(-c3cccc4c3ccc3c5ccccc5ccc43)n2)cc1]

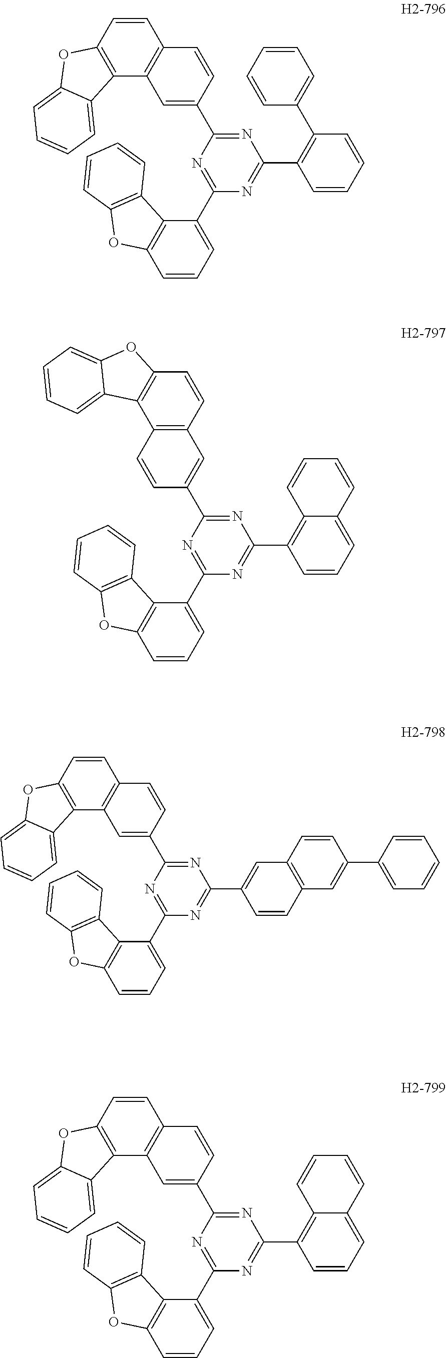 [SMILES: c1ccc(-c2ccccc2-c2nc(-c3ccc4ccc5oc6ccccc6c5c4c3)nc(-c3cccc4oc5ccccc5c34)n2)cc1 ; c1ccc2c(-c3nc(-c4ccc5c(ccc6oc7ccccc7c65)c4)nc(-c4cccc5oc6ccccc6c45)n3)cccc2c1 ; c1ccc(-c2ccc3cc(-c4nc(-c5ccc6ccc7oc8ccccc8c7c6c5)nc(-c5cccc6oc7ccccc7c56)n4)ccc3c2)cc1 ; c1ccc2c(-c3nc(-c4ccc5ccc6oc7ccccc7c6c5c4)nc(-c4cccc5oc6ccccc6c45)n3)cccc2c1]

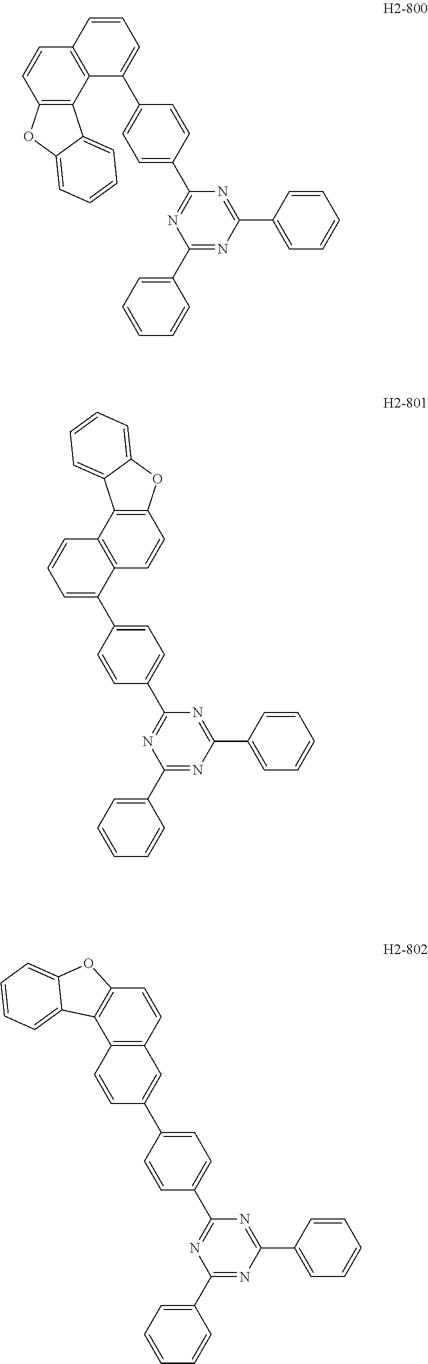 [SMILES: c1ccc(-c2nc(-c3ccccc3)nc(-c3ccc(-c4cccc5ccc6oc7ccccc7c6c45)cc3)n2)cc1 ; c1ccc(-c2nc(-c3ccccc3)nc(-c3ccc(-c4cccc5c4ccc4oc6ccccc6c45)cc3)n2)cc1 ; c1ccc(-c2nc(-c3ccccc3)nc(-c3ccc(-c4ccc5c(ccc6oc7ccccc7c65)c4)cc3)n2)cc1]

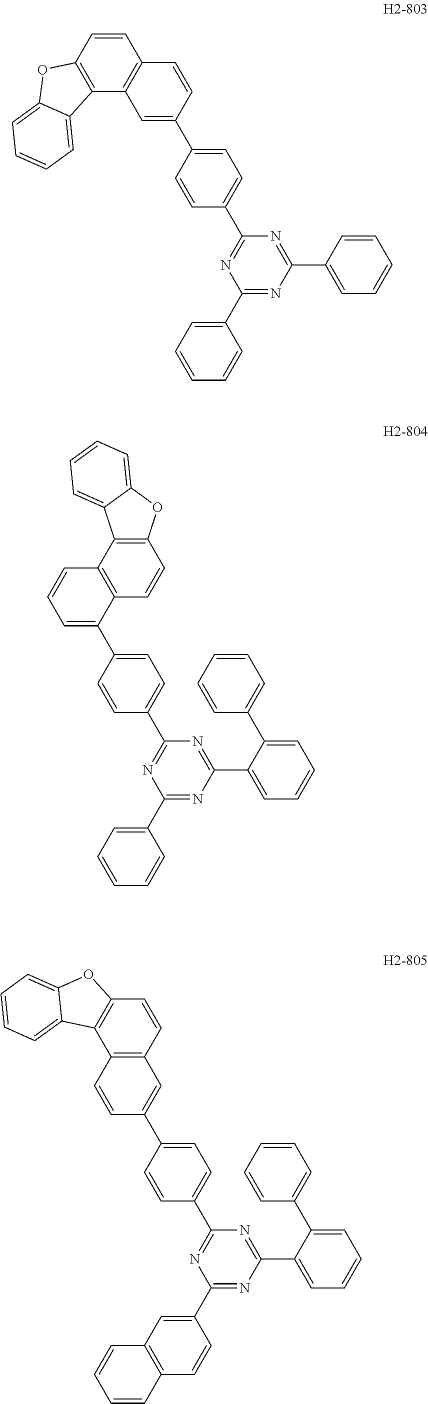 [SMILES: c1ccc(-c2nc(-c3ccccc3)nc(-c3ccc(-c4ccc5ccc6oc7ccccc7c6c5c4)cc3)n2)cc1 ; c1ccc(-c2nc(-c3ccc(-c4cccc5c4ccc4oc6ccccc6c45)cc3)nc(-c3ccccc3-c3ccccc3)n2)cc1 ; c1ccc(-c2ccccc2-c2nc(-c3ccc(-c4ccc5c(ccc6oc7ccccc7c65)c4)cc3)nc(-c3ccc4ccccc4c3)n2)cc1]

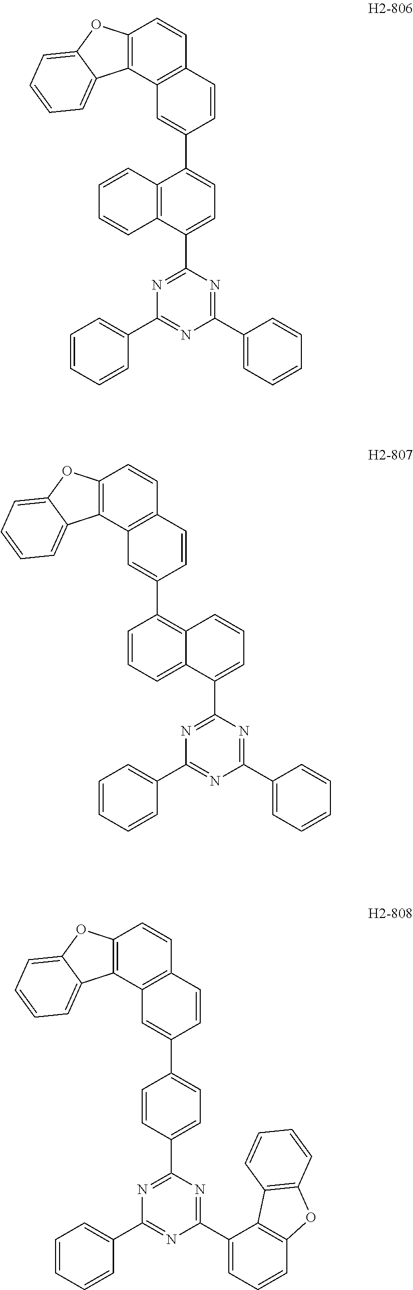 [SMILES: c1ccc(-c2nc(-c3ccccc3)nc(-c3ccc(-c4ccc5ccc6oc7ccccc7c6c5c4)c4ccccc34)n2)cc1 ; c1ccc(-c2nc(-c3ccccc3)nc(-c3cccc4c(-c5ccc6ccc7oc8ccccc8c7c6c5)cccc34)n2)cc1 ; c1ccc(-c2nc(-c3ccc(-c4ccc5ccc6oc7ccccc7c6c5c4)cc3)nc(-c3cccc4oc5ccccc5c34)n2)cc1]

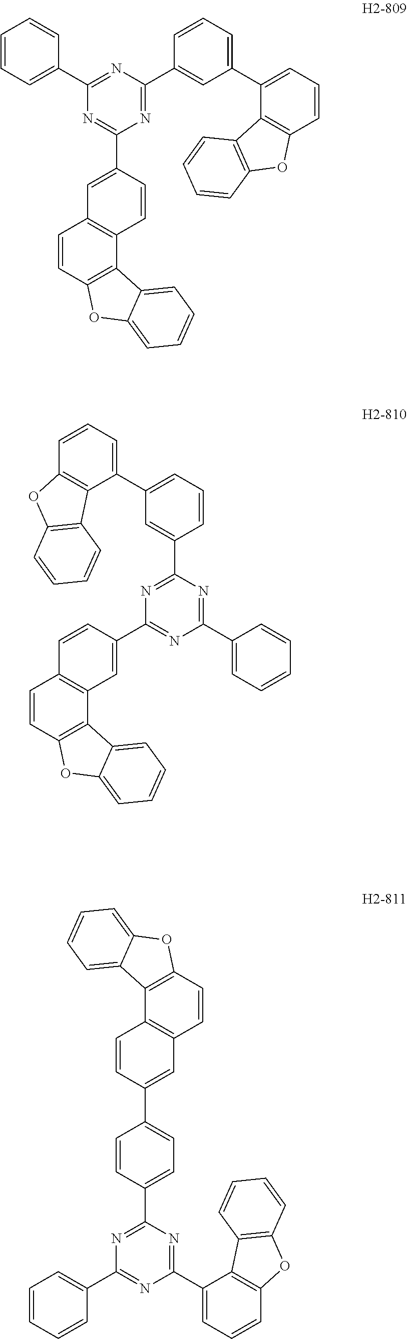 [SMILES: c1ccc(-c2nc(-c3cccc(-c4cccc5oc6ccccc6c45)c3)nc(-c3ccc4c(ccc5oc6ccccc6c54)c3)n2)cc1 ; c1ccc(-c2nc(-c3cccc(-c4cccc5oc6ccccc6c45)c3)nc(-c3ccc4ccc5oc6ccccc6c5c4c3)n2)cc1 ; c1ccc(-c2nc(-c3ccc(-c4ccc5c(ccc6oc7ccccc7c65)c4)cc3)nc(-c3cccc4oc5ccccc5c34)n2)cc1]

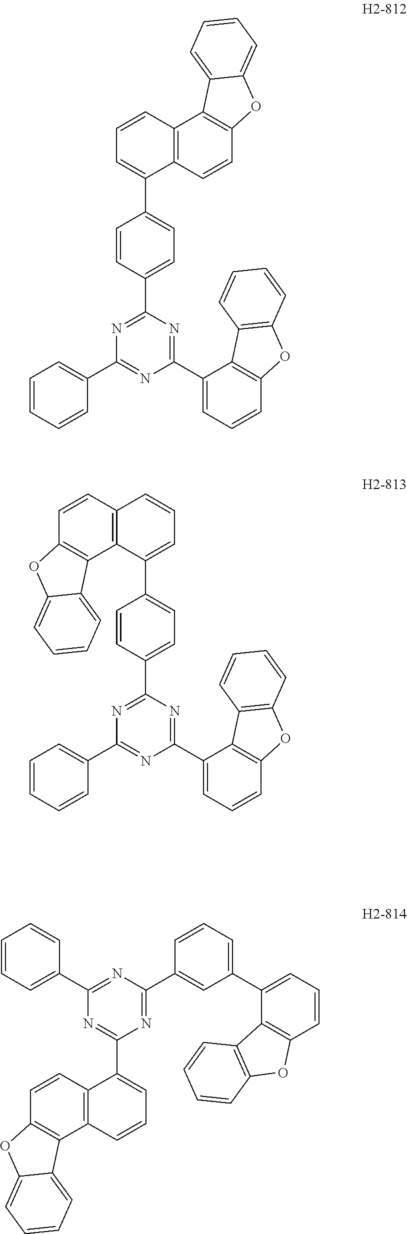 [SMILES: c1ccc(-c2nc(-c3ccc(-c4cccc5c4ccc4oc6ccccc6c45)cc3)nc(-c3cccc4oc5ccccc5c34)n2)cc1 ; c1ccc(-c2nc(-c3ccc(-c4cccc5ccc6oc7ccccc7c6c45)cc3)nc(-c3cccc4oc5ccccc5c34)n2)cc1 ; c1ccc(-c2nc(-c3cccc(-c4cccc5oc6ccccc6c45)c3)nc(-c3cccc4c3ccc3oc5ccccc5c34)n2)cc1]

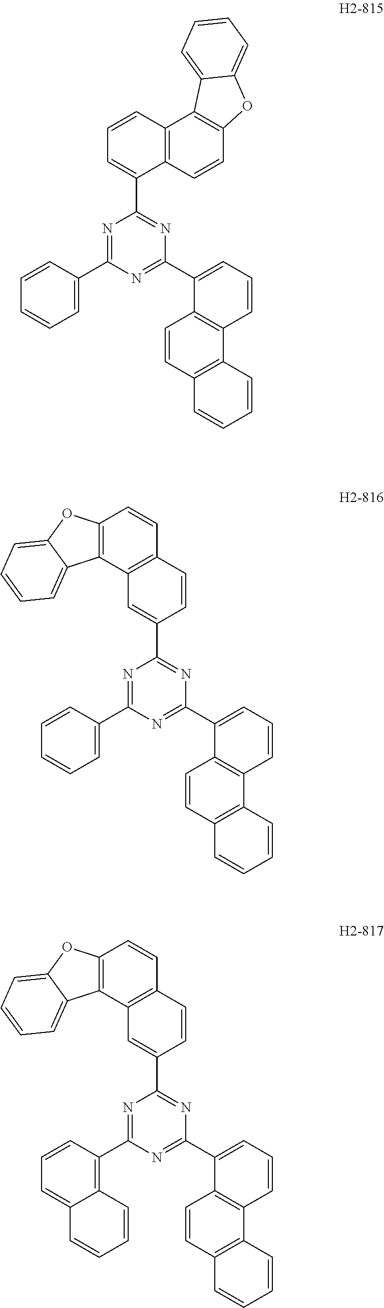 [SMILES: c1ccc(-c2nc(-c3cccc4c3ccc3ccccc34)nc(-c3cccc4c3ccc3oc5ccccc5c34)n2)cc1 ; c1ccc(-c2nc(-c3ccc4ccc5oc6ccccc6c5c4c3)nc(-c3cccc4c3ccc3ccccc34)n2)cc1 ; c1ccc2c(-c3nc(-c4ccc5ccc6oc7ccccc7c6c5c4)nc(-c4cccc5c4ccc4ccccc45)n3)cccc2c1]

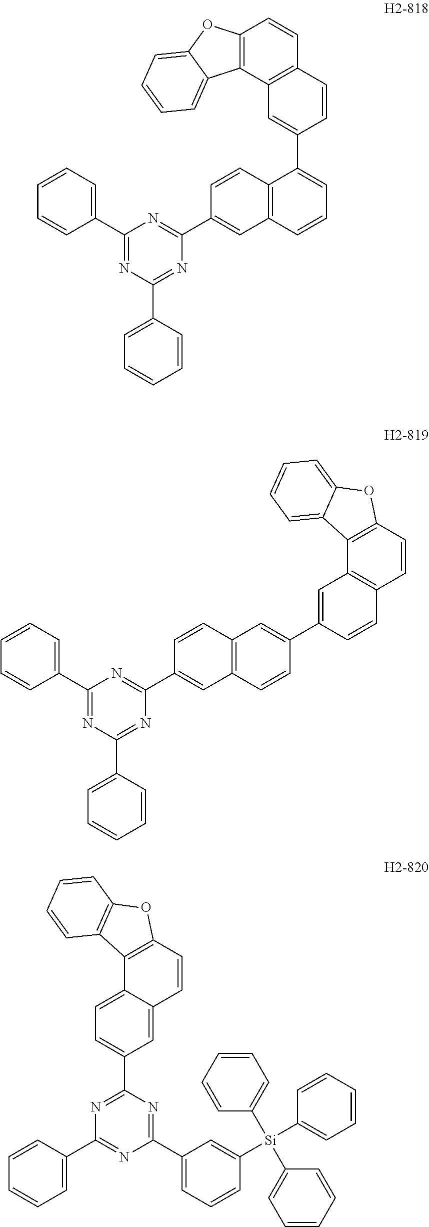

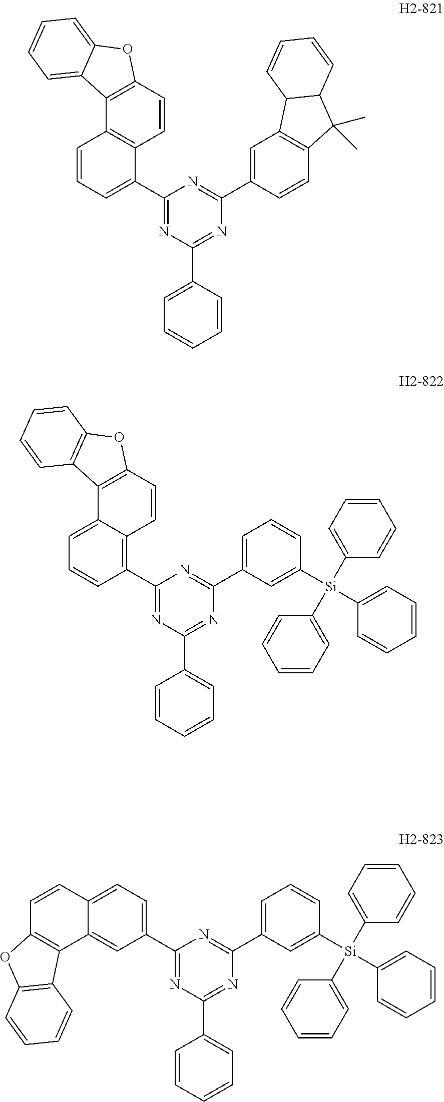 [SMILES: CC1(C)c2ccc(-c3nc(-c4ccccc4)nc(-c4cccc5c4ccc4oc6ccccc6c45)n3)cc2C2C=CC=CC21 ; c1ccc(-c2nc(-c3cccc([Si](c4ccccc4)(c4ccccc4)c4ccccc4)c3)nc(-c3cccc4c3ccc3oc5ccccc5c34)n2)cc1 ; c1ccc(-c2nc(-c3cccc([Si](c4ccccc4)(c4ccccc4)c4ccccc4)c3)nc(-c3ccc4ccc5oc6ccccc6c5c4c3)n2)cc1]

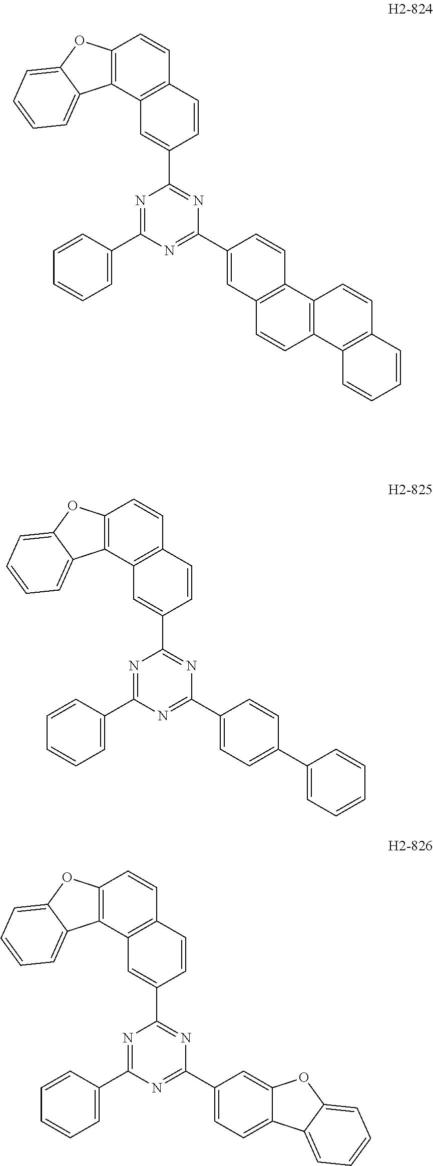 [SMILES: c1ccc(-c2nc(-c3ccc4c(ccc5c6ccccc6ccc45)c3)nc(-c3ccc4ccc5oc6ccccc6c5c4c3)n2)cc1 ; c1ccc(-c2ccc(-c3nc(-c4ccccc4)nc(-c4ccc5ccc6oc7ccccc7c6c5c4)n3)cc2)cc1 ; c1ccc(-c2nc(-c3ccc4c(c3)oc3ccccc34)nc(-c3ccc4ccc5oc6ccccc6c5c4c3)n2)cc1]

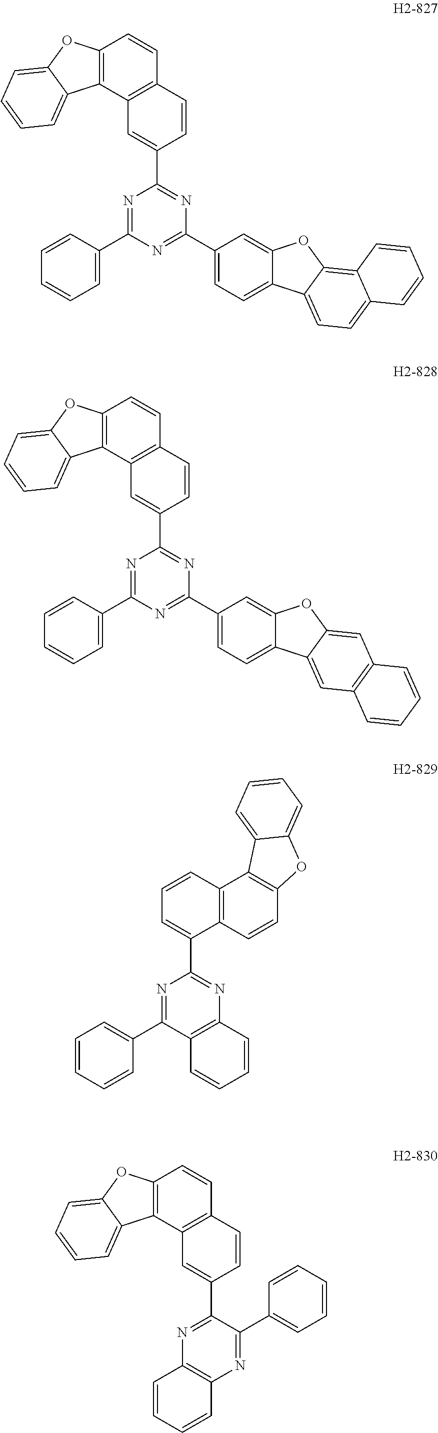 [SMILES: c1ccc(-c2nc(-c3ccc4c(c3)oc3c5ccccc5ccc43)nc(-c3ccc4ccc5oc6ccccc6c5c4c3)n2)cc1 ; c1ccc(-c2nc(-c3ccc4c(c3)oc3cc5ccccc5cc34)nc(-c3ccc4ccc5oc6ccccc6c5c4c3)n2)cc1 ; c1ccc(-c2nc(-c3cccc4c3ccc3oc5ccccc5c34)nc3ccccc23)cc1 ; c1ccc(-c2nc3ccccc3nc2-c2ccc3ccc4oc5ccccc5c4c3c2)cc1]

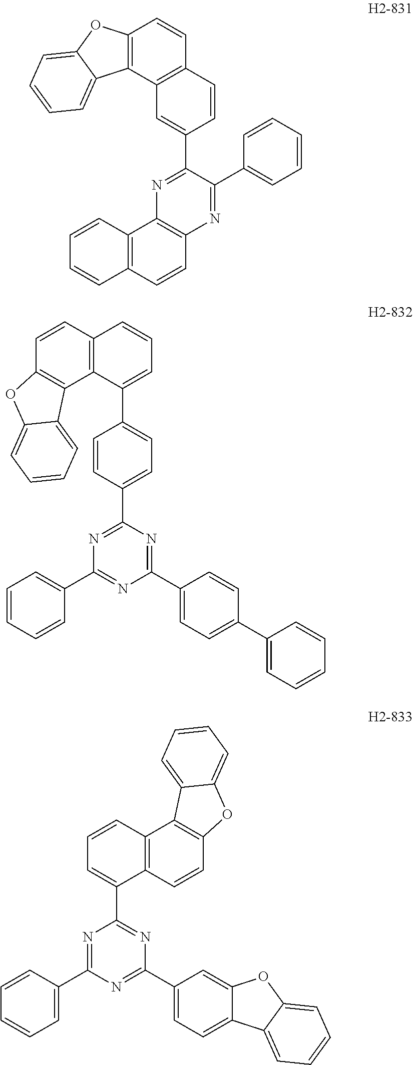

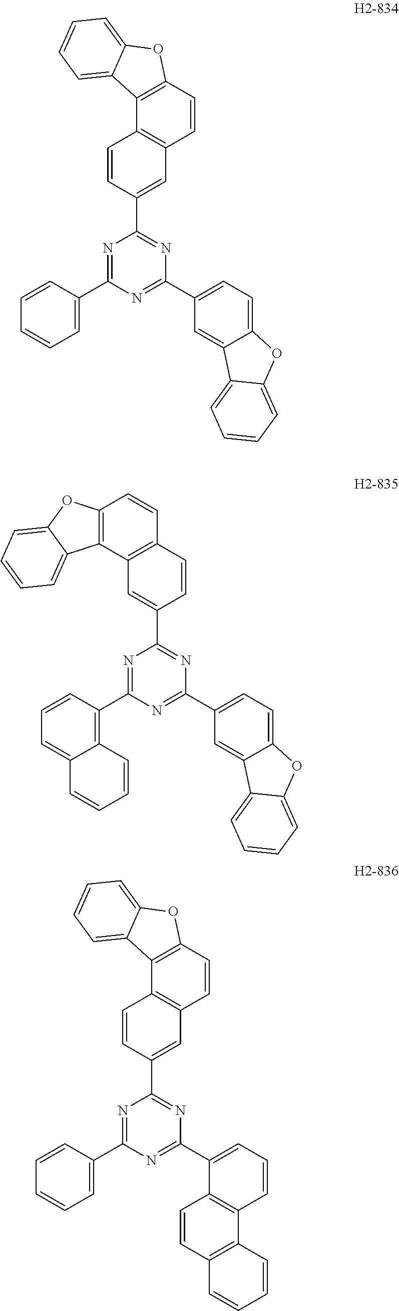 [SMILES: c1ccc(-c2nc(-c3ccc4c(ccc5oc6ccccc6c54)c3)nc(-c3ccc4oc5ccccc5c4c3)n2)cc1 ; c1ccc2c(-c3nc(-c4ccc5oc6ccccc6c5c4)nc(-c4ccc5ccc6oc7ccccc7c6c5c4)n3)cccc2c1 ; c1ccc(-c2nc(-c3ccc4c(ccc5oc6ccccc6c54)c3)nc(-c3cccc4c3ccc3ccccc34)n2)cc1]

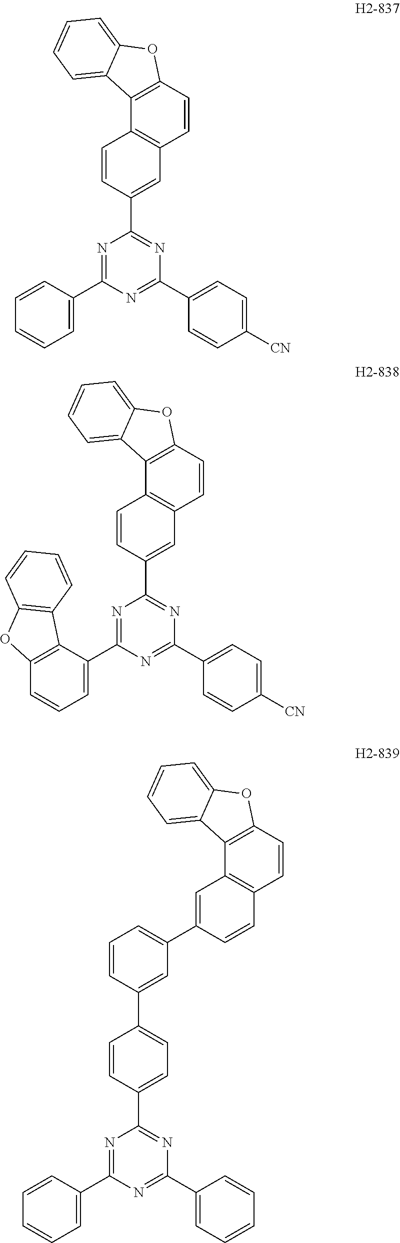 [SMILES: N#Cc1ccc(-c2nc(-c3ccccc3)nc(-c3ccc4c(ccc5oc6ccccc6c54)c3)n2)cc1 ; N#Cc1ccc(-c2nc(-c3ccc4c(ccc5oc6ccccc6c54)c3)nc(-c3cccc4oc5ccccc5c34)n2)cc1 ; c1ccc(-c2nc(-c3ccccc3)nc(-c3ccc(-c4cccc(-c5ccc6ccc7oc8ccccc8c7c6c5)c4)cc3)n2)cc1]

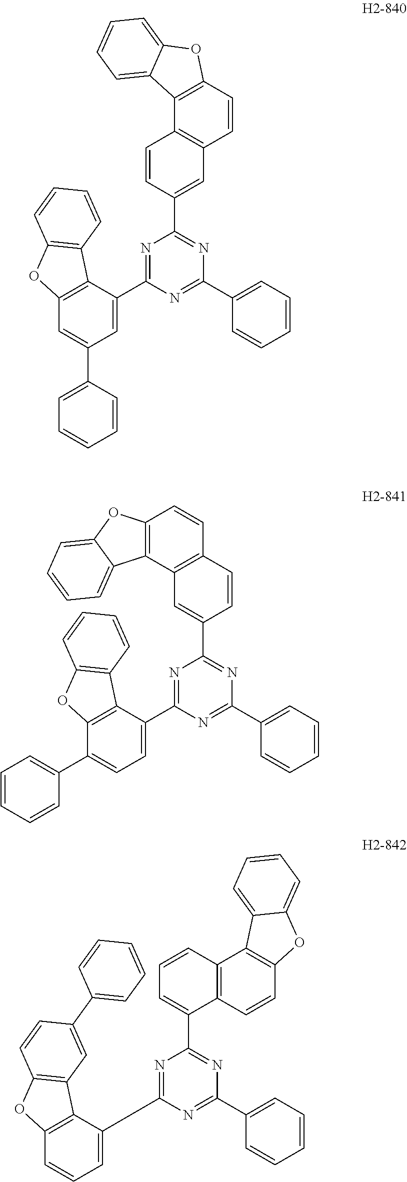 [SMILES: c1ccc(-c2cc(-c3nc(-c4ccccc4)nc(-c4ccc5c(ccc6oc7ccccc7c65)c4)n3)c3c(c2)oc2ccccc23)cc1 ; c1ccc(-c2nc(-c3ccc4ccc5oc6ccccc6c5c4c3)nc(-c3ccc(-c4ccccc4)c4oc5ccccc5c34)n2)cc1 ; c1ccc(-c2ccc3oc4cccc(-c5nc(-c6ccccc6)nc(-c6cccc7c6ccc6oc8ccccc8c67)n5)c4c3c2)cc1]

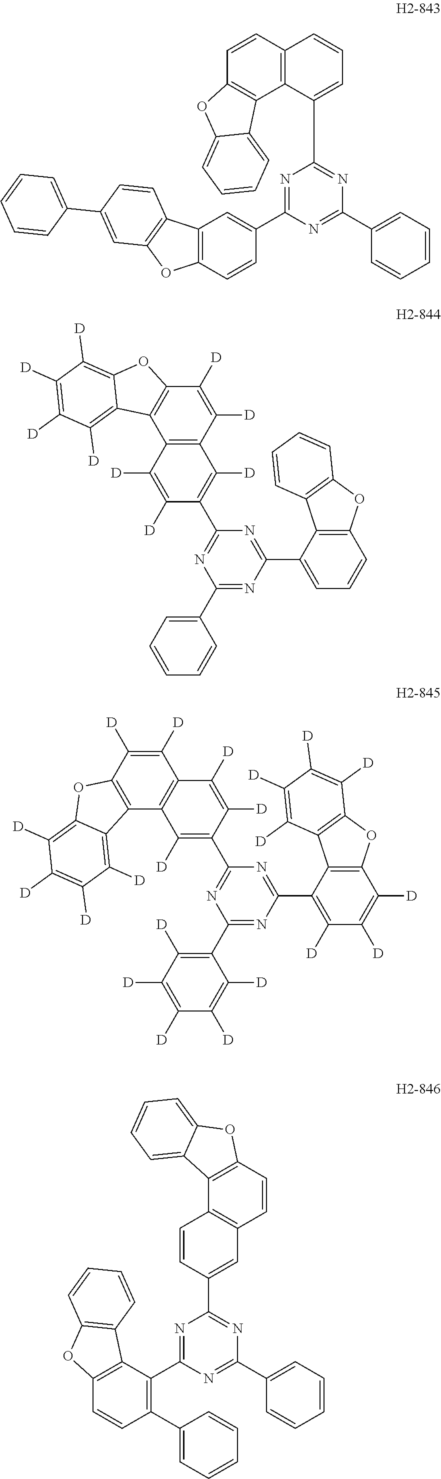 [SMILES: c1ccc(-c2ccc3c(c2)oc2ccc(-c4nc(-c5ccccc5)nc(-c5cccc6ccc7oc8ccccc8c7c56)n4)cc23)cc1 ; [2H]c1c([2H])c([2H])c2c(oc3c([2H])c([2H])c4c([2H])c(-c5nc(-c6ccccc6)nc(-c6cccc7oc8ccccc8c67)n5)c([2H])c([2H])c4c32)c1[2H] ; [2H]c1c([2H])c([2H])c(-c2nc(-c3c([2H])c([2H])c4c([2H])c([2H])c5oc6c([2H])c([2H])c([2H])c([2H])c6c5c4c3[2H])nc(-c3c([2H])c([2H])c([2H])c4oc5c([2H])c([2H])c([2H])c([2H])c5c34)n2)c([2H])c1[2H] ; c1ccc(-c2nc(-c3ccc4c(ccc5oc6ccccc6c54)c3)nc(-c3c(-c4ccccc4)ccc4oc5ccccc5c34)n2)cc1]

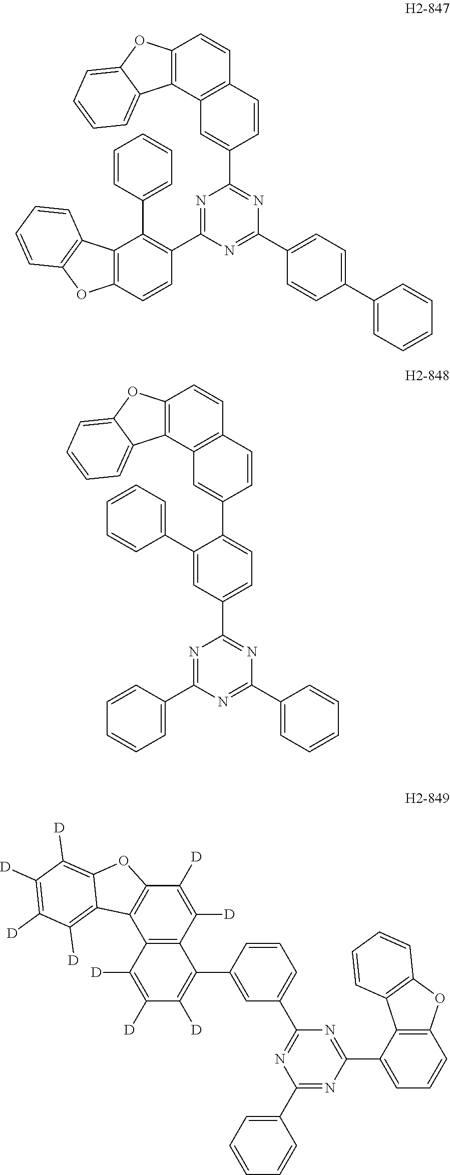 [SMILES: c1ccc(-c2ccc(-c3nc(-c4ccc5ccc6oc7ccccc7c6c5c4)nc(-c4ccc5oc6ccccc6c5c4-c4ccccc4)n3)cc2)cc1 ; c1ccc(-c2nc(-c3ccccc3)nc(-c3ccc(-c4ccc5ccc6oc7ccccc7c6c5c4)c(-c4ccccc4)c3)n2)cc1 ; [2H]c1c([2H])c([2H])c2c(oc3c([2H])c([2H])c4c(-c5cccc(-c6nc(-c7ccccc7)nc(-c7cccc8oc9ccccc9c78)n6)c5)c([2H])c([2H])c([2H])c4c32)c1[2H]]

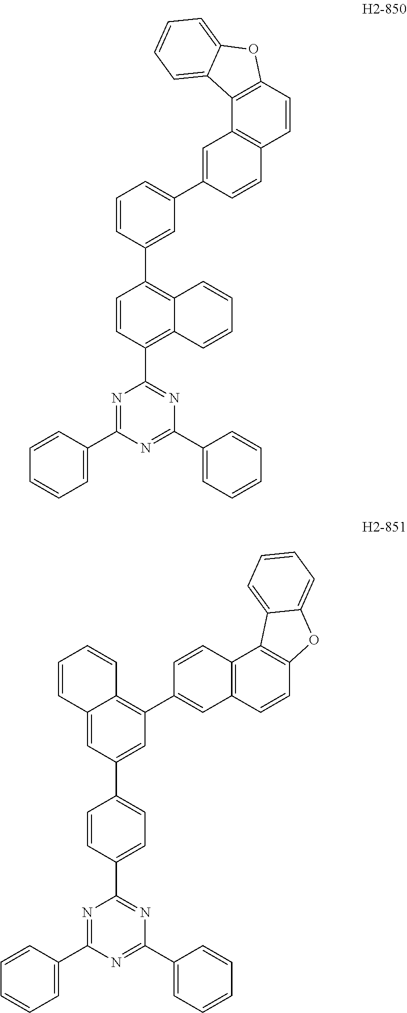 [SMILES: c1ccc(-c2nc(-c3ccccc3)nc(-c3ccc(-c4cccc(-c5ccc6ccc7oc8ccccc8c7c6c5)c4)c4ccccc34)n2)cc1 ; c1ccc(-c2nc(-c3ccccc3)nc(-c3ccc(-c4cc(-c5ccc6c(ccc7oc8ccccc8c76)c5)c5ccccc5c4)cc3)n2)cc1]

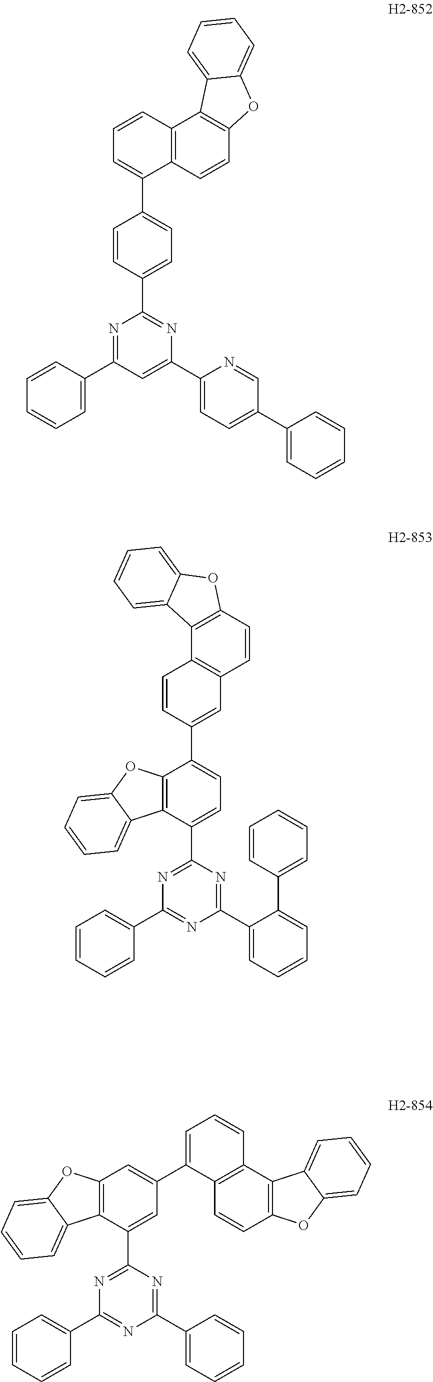 [SMILES: c1ccc(-c2ccc(-c3cc(-c4ccccc4)nc(-c4ccc(-c5cccc6c5ccc5oc7ccccc7c56)cc4)n3)nc2)cc1 ; c1ccc(-c2nc(-c3ccccc3-c3ccccc3)nc(-c3ccc(-c4ccc5c(ccc6oc7ccccc7c65)c4)c4oc5ccccc5c34)n2)cc1 ; c1ccc(-c2nc(-c3ccccc3)nc(-c3cc(-c4cccc5c4ccc4oc6ccccc6c45)cc4oc5ccccc5c34)n2)cc1]

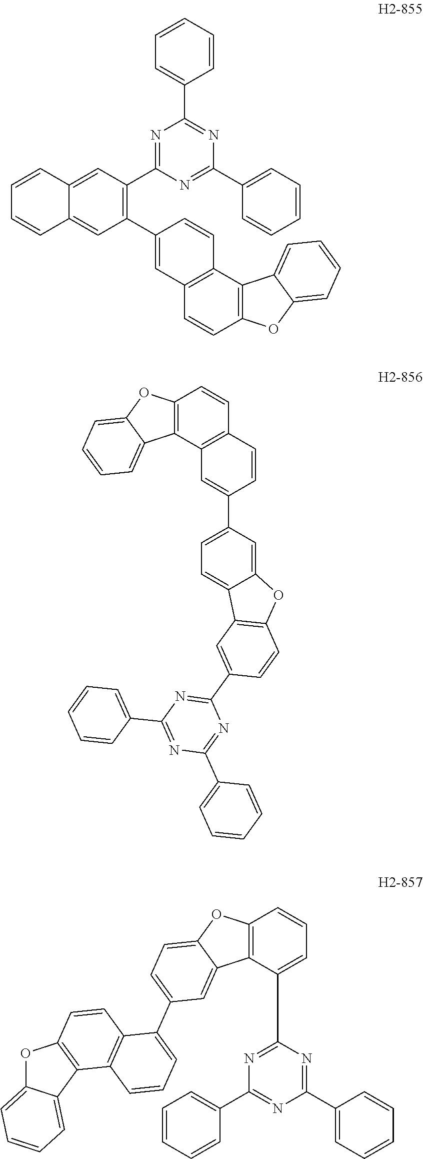 [SMILES: c1ccc(-c2nc(-c3ccccc3)nc(-c3cc4ccccc4cc3-c3ccc4c(ccc5oc6ccccc6c54)c3)n2)cc1 ; c1ccc(-c2nc(-c3ccccc3)nc(-c3ccc4oc5cc(-c6ccc7ccc8oc9ccccc9c8c7c6)ccc5c4c3)n2)cc1 ; c1ccc(-c2nc(-c3ccccc3)nc(-c3cccc4oc5ccc(-c6cccc7c6ccc6oc8ccccc8c67)cc5c34)n2)cc1]

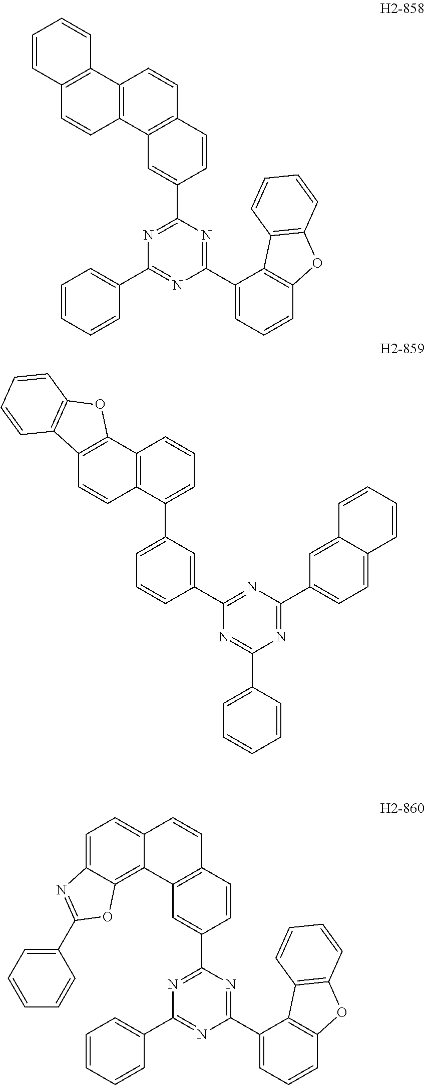 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3cccc(-c4cccc5c4ccc4c6ccccc6oc54)c3)nc(-c3ccc4ccccc4c3)n2)cc1 ; c1ccc(-c2nc(-c3ccc4ccc5ccc6nc(-c7ccccc7)oc6c5c4c3)nc(-c3cccc4oc5ccccc5c34)n2)cc1]

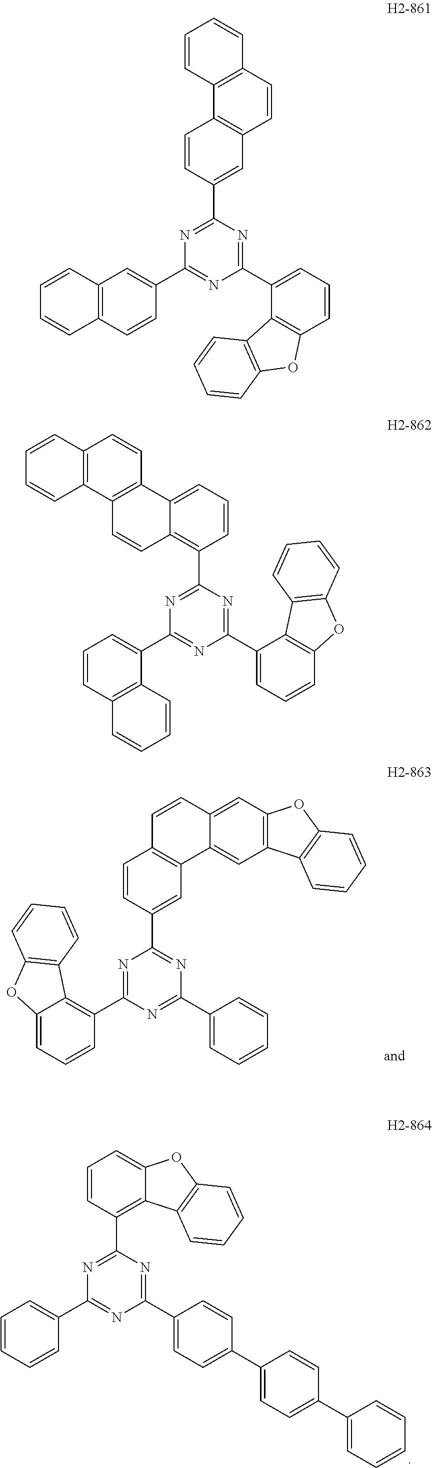 [SMILES: c1ccc2cc(-c3nc(-c4ccc5c(ccc6ccccc65)c4)nc(-c4cccc5oc6ccccc6c45)n3)ccc2c1 ; c1ccc2c(-c3nc(-c4cccc5c4ccc4c6ccccc6ccc54)nc(-c4cccc5oc6ccccc6c45)n3)cccc2c1 ; c1ccc(-c2nc(-c3ccc4ccc5cc6oc7ccccc7c6cc5c4c3)nc(-c3cccc4oc5ccccc5c34)n2)cc1 ; c1ccc(-c2ccc(-c3ccc(-c4nc(-c5ccccc5)nc(-c5cccc6oc7ccccc7c56)n4)cc3)cc2)cc1]

12. An organic electroluminescent device comprising an anode, a cathode, and at least one light-emitting layer between the anode and the cathode, wherein at least one of the light-emitting layers comprises the plurality of host materials according to claim 1.
